(19) United States
(12) Patent Application Publication
(10) Pub. No.: US 2025/0051849 A1
(43) Pub. Date: Feb. 13, 2025

(54) LIQUID BIOPSY ASSAYS FOR DETECTING NRG1 FUSION POLYNUCLEOTIDES

(71) Applicant: MERUS N.V., Utrecht (NL)

(72) Inventors: Ernesto Isaac WASSERMAN, Utrecht (NL); Jeroen Jilles LAMMERTS VAN BUEREN, Utrecht (NL)

(21) Appl. No.: 18/566,980

(22) PCT Filed: Jun. 1, 2022
(86) PCT No.: PCT/NL2022/050300

§ 371 Date: Dec. 4, 2023

(30) Foreign Application Priority Data
Jun. 3, 2021 (NL) 2028383
Dec. 3, 2021 (NL) 2030005

(51) Int. Cl. C12Q 1/6886

(52) U.S. Cl. CPC C12Q 1/6886; C12Q 2600/156

(57) ABSTRACT

This invention relates to liquid biopsy assays for the detection of neuregulin-1 (NRG1) fusion polynucleotides, particularly their use for diagnosing or treating disease, such as cancer. Novel neuregulin-1 (NRG1) fusion polynucleotides are also disclosed, as are kits for determining the presence or absence of an NRG1 fusion polynucleotide in a liquid biopsy sample. Methods of treating disease, such as cancer, are also disclosed.

### TECHNICAL FIELD

This invention relates to liquid biopsy assays for the detection of neuregulin-1 (NRG1) fusion polynucleotides, particularly their use for diagnosing or treating disease, such as cancer. Novel neuregulin-1 (NRG1) fusion polynucleotides are also disclosed.

### BACKGROUND

NRG1 fusions genes are rare but actionable oncogenic drivers that are present in solid tumors. Various NRG1 fusion genes are described in Dhanasekaran et al. (Nature Communications, 2014, 5:5893), and in Jonna et al. (Clin Cancer Res, 2019, 25 (16): 4966-4972). Besides occurring rarely, another characteristic of NRG1 fusions is that they can result from an unpredictable chromosomal rearrangement event that involves NRG1.

Standard means for identifying NRG1 fusions are by performing molecular screening of biopsies of solid tumours, which typically involves impactful disruption of the physical integrity of the skin, and possibly internal organs, to reach the tumor under investigation, and may still not identify the presence of many NRG1 fusions. Accordingly, there is a need for novel assays for detecting and diagnosing cancer in NRG1 fusion polynucleotides, particularly novel NRG1 fusion polynucleotides.

### SUMMARY OF THE INVENTION

The invention involves the determination of the presence or absence of one or more NRG1 fusion polynucleotides in liquid biopsy samples and the use of such assays to diagnose, prognose and treat cancer.

The invention provides:
a method for determining the presence or absence of an NRG1 fusion polynucleotide in a sample, the method comprising:

1) providing a liquid biopsy sample, and

2) determining the presence or absence of the NRG1 fusion polynucleotide in the sample,

wherein the NRG fusion polynucleotide is a fusion of an NRG1 polynucleotide with a second polynucleotide, which second polynucleotide does not encode NRG1;
a method for measuring the amount or concentration of an NRG1 fusion polynucleotide in a sample, comprising conducting the method the invention and, if the NRG1 fusion polynucleotide is present, determining its amount or concentration;
a method for determining the presence or absence of two or more NRG1 fusion polynucleotides in a sample, the method comprising:

1) providing a liquid biopsy sample, and

2) determining the presence or absence of the two or more NRG1 fusion polynucleotides in the sample, wherein the NRG fusion polynucleotides are fusions of an NRG1 polynucleotide with a second polynucleotide, which second polynucleotide does not encode NRG1;
a kit for determining the presence or absence of an NRG1 fusion polynucleotide in a liquid biopsy sample, comprising (a) a pair of forward and reverse polynucleotide primers which specifically hybridise to the NRG1 fusion polynucleotide and/or (b) a polynucleotide probe which specifically hybridises to the NRG1 fusion polynucleotide;
a method of determining whether or not a subject has cancer or is prone to developing cancer, wherein the method comprises conducting a method according to the invention on a sample from the subject and thereby determining whether or not the subject has cancer or is prone to developing cancer;
a method of treating cancer in a subject identified as having cancer using a method of the invention, comprising administering to the subject a therapeutically effective amount of a cancer treatment and thereby treating the cancer in the subject; and
a substance or composition for use in a method of treating cancer in a subject identified as having cancer using a method of the invention.

Throughout the description and claims of this specification, the words “comprise” and “contain” and variations of the words, for example “comprising” and “comprises”, mean “including but not limited to”, and do not exclude other components, integers or steps. Moreover the singular encompasses the plural unless the context otherwise requires: in particular, where the indefinite article is used, the specification is to be understood as contemplating plurality as well as singularity, unless the context requires otherwise.

Disclosed features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### DETAILED DESCRIPTION

### Determining the Presence or Absence of an NRG1 Fusion Polynucleotide

The invention provides a method of determining the presence or absence of an NRG1 fusion polynucleotide in a sample. In all instances herein, determining is interchangeable with detecting. The methods of the invention may be for detecting the presence or absence of an NRG1 fusion polynucleotide in a sample. Also, in all instances, the methods of the invention may be for determining/detecting/identifying whether or not a sample contains or comprises an NRG1 fusion polynucleotide.

Methods for determining the presence or absence of polynucleotides in samples are well known in the art. Specific methods are discussed in more detail below.

### Fusion Polynucleotides

Fusion polynucleotides according to the present invention have the general formula 5′-A-B-3′, wherein A is a first polynucleotide and B is a second polynucleotide. The meeting point between the first polynucleotide and the second polynucleotide is known as the “fusion junction”. For example, if the fusion polynucleotide had a length of 20 nucleotides, the first polynucleotide consisted of nucleotides 1-10 and the second polynucleotide consisted of nucleotides 11-20, the fusion junction would be found between nucleotides 10 and 11. Preferably the method of the invention comprises determining the presence or absence of the NRG1 fusion polynucleotide in the sample by determining the presence or absence of a fusion junction between the NRG1 polynucleotide and the second polynucleotide. Specific methods for doing this are discussed in more detail below.

Preferably the NRG1 fusion polynucleotide is an oncogenic NRG1 fusion polynucleotide. Oncogenic NRG1 fusion polynucleotides are associated with or indicative of a cancer in a subject. The cancer may be any of those discussed below.

The fusion polynucleotides of the present invention comprise an NRG1 polynucleotide. Preferably the NRG1 polynucleotide is 3′ to the second polynucleotide. In other words, the second polynucleotide is preferably 5′ to the NRG1 polynucleotide. Thus, using the general formula above, the NRG1 fusion polynucleotides of the present invention preferably have the general formula 5′-A-B-3′, wherein A is a second polynucleotide and B is an NRG1 polynucleotide. Preferably the fusion between the NRG1 polynucleotide and the second polynucleotide is an operably linked fusion. More preferably the fusion between the NRG1 polynucleotide and the second polynucleotide is an in-frame fusion. Operably linked in this context means a covalent connection between two polynucleotides in a manner that permits transcription into a protein. In one aspect, the fusion between the NRG1 polynucleotide and the second polynucleotide involves a fusion which is not in the protein coding sequence of any of the fused polynucleotides but, wherein as a result, the NRG1 polynucleotide has become transcriptionally controlled by the promoter of the first polynucleotide. Thus rather in this aspect, the fusion involves a fusion involving the untranslated region (i.e. the 5′ UTR) of NRG1 and the untranslated region (i.e. the 5′ UTR) of a second polynucleotide. In particular, and according to the general formula given of 5′-A-B-3′, A comprises an untranslated exon, or an untranslated portion thereof, and B comprises an untranslated portion of exon 1 of NRG1. Thus in disclosed embodiments, the NRG fusion polynucleotide is a fusion of an NRG1 polynucleotide with a second polynucleotide, wherein the fused NRG1 polynucleotide comprises an untranslated portion of exon 1 and the second polynucleotide comprises an untranslated exon or an untranslated portion thereof, preferably including exon 1 or more untranslated exons. In particular, the second polynucleotide in this aspect is a DAAM1 polynucleotide.

The second polynucleotide is not an NRG1 polynucleotide. The second polynucleotide does not encode NRG1. The second polynucleotide can be any polynucleotide having an active promoter and resulting in the transcription and translation of an NRG1 fusion polypeptide. In an embodiment, the second polynucleotide is selected from a VAPB polynucleotide, a CADM1 polynucleotide, a CD44 polynucleotide, a SLC3A2 polynucleotide, a VTCN1 polynucleotide, a CDH1 polynucleotide, a CXADR polynucleotide, a GTF2E2 polynucleotide, a CSMD1 polynucleotide, a PTN polynucleotide, a ST14 polynucleotide, THBS1 polynucleotide, an AGRN polynucleotide, a PVALP polynucleotide, an APP polynucleotide, a WRN polynucleotide, a DAAM1 polynucleotide, an ASPH polynucleotide, a NOTCH2 polynucleotide, a CD74 polynucleotide, a SDC4 polynucleotide, a SLC4A4 polynucleotide, a ZFAT polynucleotide, a DSCAML1 polynucleotide, a THBS1 polynucleotide, a RBPMS polynucleotide and an ATP1B1 polynucleotide.

Disclosed fusion polynucleotides disclosed herein include VAPB-NRG1 (i.e. comprising or consisting of a VAPB polynucleotide and an NRG1 polynucleotide), CADM1-NRG1 (i.e. comprising or consisting of a CADM1 polynucleotide and an NRG1 polynucleotide), CD44-NRG1 (i.e. comprising or consisting of a CD44 polynucleotide and an NRG1 polynucleotide), SLC3A2-NRG1 (i.e. comprising or consisting of a SLC3A2 polynucleotide and an NRG1 polynucleotide), VTCN1-NRG1 (i.e. comprising or consisting of a VTCN1 polynucleotide and an NRG1 polynucleotide), CDH1-NRG1 (i.e. comprising or consisting of a CDH1 polynucleotide and an NRG1 polynucleotide), CXADR-NRG1 (i.e. comprising or consisting of a CXADR polynucleotide and an NRG1 polynucleotide, GTF2E2-NRG1 (i.e. comprising or consisting of a GTF2E2 polynucleotide and an NRG1 polynucleotide, CSMD1-NRG1 (i.e. comprising or consisting of a CSMD1 polynucleotide and an NRG1 polynucleotide, PTN-NRG1 (i.e. comprising or consisting of a PTN polynucleotide and an NRG1 polynucleotide, ST14-NRG1 (i.e. comprising or consisting of an ST14 polynucleotide and an NRG1 polynucleotide, THBS1-NRG1 (i.e. comprising or consisting of a THBS1 polynucleotide and an NRG1 polynucleotide, AGRN-NRG1 (i.e. comprising or consisting of an AGRN polynucleotide and an NRG1 polynucleotide, PVALP-NRG1 (i.e. comprising or consisting of a PVALP polynucleotide and an NRG1 polynucleotide), APP-NRG1 (i.e. comprising or consisting of a APP polynucleotide and an NRG1 polynucleotide), WRN-NRG1 (i.e. comprising or consisting of a WRN polynucleotide and an NRG1 polynucleotide), DAAM1-NRG1 (i.e. comprising or consisting of a DAAM1 polynucleotide and an NRG1 polynucleotide), ASPH-NRG1 (i.e. comprising or consisting of a ASPH polynucleotide and an NRG1 polynucleotide), NOTCH2-NRG1 (i.e. comprising or consisting of a NOTCH2 polynucleotide and an NRG1 polynucleotide), CD74-NRG1 (i.e. comprising or consisting of a CD74 polynucleotide and an NRG1 polynucleotide), SDC4-NRG1 (i.e. comprising or consisting of a SDC4 polynucleotide and an NRG1 polynucleotide), SLC4A4-NRG1 (i.e. comprising or consisting of a SLC4A4 polynucleotide and an NRG1 polynucleotide), ZFAT-NRG1 (i.e. comprising or consisting of a ZFAT polynucleotide and an NRG1 polynucleotide) and DSCAML1-NRG1 (i.e. comprising or consisting of a DSCAML1 polynucleotide and an NRG1 polynucleotide), THBS1-NRG1 (i.e. comprising or consisting of a THBS1 polynucleotide and an NRG1 polynucleotide), RBPMS-NRG1 polynucleotide (i.e. comprising or consisting of a RBPMS polynucleotide and an NRG1 polynucleotide) and ATP1B1-NRG1 (i.e. comprising or consisting of a ATP1B1 polynucleotide and an NRG1 polynucleotide).

### NRG1 Polynucleotide Sequences

The NRG1-gene codes for various isoforms of NRG1. Various isoforms and their expected function are described in Adelaide et al. Genes Chromosomes Cancer, August; 37 (4): 333-45 (2003). GGF and GGF2 isoforms contain a kringle-like sequence plus Ig and EGF-like domains; and the SMDF isoform shares only the EGF-like domain with other isoforms. The receptors for NRG1 isoforms are the ErbB family of tyrosine kinase transmembrane receptors. The family is also referred to as the human epidermal growth factor (EGF) receptor family (HER). The family has four members: ErbB (Erythroblastoma)-1, ErbB-2, ErbB-3 and ErbB-4. The receptors (reviewed in Yarden and Pines 2012) are widely expressed on epithelial cells. Upregulation of HER receptors or their ligands, such as heregulin (HRG) or epidermal growth factor (EGF), is a frequent event in human cancer (Wilson, Fridlyand et al. 2012). Overexpression of ErbB-1 and ErbB-2 in particular occurs in epithelial tumors and is associated with tumor invasion, metastasis, resistance to chemotherapy, and poor prognosis (Zhang, Berezov et al. 2007). In the normal breast, ErbB-3 has been shown to be important in the growth and differentiation of luminal epithelium. For instance, loss/inhibition of ErbB-3 results in selective expansion of the basal over the luminal epithelium (Balko, Miller et al. 2012). Binding of ligand to the extracellular domain of a receptor tyrosine kinase induces receptor dimerization, both between the same (homodimerization) and different (heterodimerization) receptor subtypes. Dimerization can activate the intracellular tyrosine kinase domains, which undergo autophosphorylation and, in turn, can activate a number of downstream pro-proliferative signaling pathways, including those mediated by mitogen-activated protein kinases (MAPK) and the pro-survival pathway Akt (reviewed in Yarden and Pines, 2012). ErbB-3 can be activated by engagement of its ligands. These ligands include but are not limited to neuregulin (NRG) and heregulin (HRG), and NRG1 fusions.

The NRG1-gene codes for the various isoforms of NRG1. Various isoforms and their expected function are described in Adelaide et al. (Genes Chromosomes Cancer, 2003, 37 (4): 333-45). GGF and GGF2 isoforms contain a kringle-like sequence plus Ig and EGF-like domains; and the SMDF isoform shares only the EGF-like domain with other isoforms. The receptors for NRG1 isoforms are the ErbB family of tyrosine kinase transmembrane receptors. The family is also referred to as the human epidermal growth factor (EGF) receptor family (HER). The family has four members: ErbB (Erythroblastoma)-1, ErbB-2, ErbB-3 and ErbB-4. The receptors (reviewed in Yarden and Pines, Nat Rev Cancer. 2012 Jul. 12; 12 (8): 553-63.) are widely expressed on epithelial cells. Upregulation of HER receptors or their ligands, such as heregulin (HRG) or epidermal growth factor (EGF), is a frequent event in human cancer (Wilson, Fridlyand et al., Nature. 2012 Jul. 26; 487 (7408): 505-509). Overexpression of ErbB-1 and ErbB-2 in particular occurs in epithelial tumors and is associated with tumor invasion, metastasis, resistance to chemotherapy, and poor prognosis (Zhang, H., Berezov, A., Wang, Q., Zhang, G., Drebin, J., Murali, R., et al. (2007) Erbb receptors: from oncogenes to targeted cancer therapies. J. Clin. Invest., 117, 2051-2058). In the normal breast, ErbB-3 has been shown to be important in the growth and differentiation of luminal epithelium. For instance, loss/inhibition of ErbB-3 results in selective expansion of the basal over the luminal epithelium (Balko, Miller et al., 2012 PNAS Jan. 3, 2012 109 (1) 221-226). Binding of ligand to the extracellular domain of a receptor tyrosine kinase induces receptor dimerization, both between the same (homodimerization) and different (heterodimerization) receptor subtypes. Dimerization can activate the intracellular tyrosine kinase domains, which undergo autophosphorylation and, in turn, can activate a number of downstream pro-proliferative signaling pathways, including those mediated by mitogen-activated protein kinases (MAPK) and the pro-survival pathway Akt (reviewed in Yarden and Pines, Nat Rev Cancer, 2012 Jul. 12; 12 (8): 553-63.). ErbB-3 can be activated by engagement of its ligands. These ligands include but are not limited to neuregulin (NRG) and heregulin (HRG), and NRG1 fusions.

NRG1 protein comprises an EGF-like domain. An EGF-like domain is a sequence of typically about thirty to forty amino-acid residues long of which the prototype is found in the sequence of epidermal growth factor (EGF) [PMID: 2288911, PMID: 6334307, PMID: 1522591, PMID: 6607417, PMID: 3282918, PMID: 11498013]. It is known to be present, in a more or less conserved form, in a large number of other, mostly animal proteins. A common feature of EGF-like domains is that they are found in the extracellular domain of membrane-bound proteins or in proteins known to be secreted (exception: prostaglandin G/H synthase). The EGF domain typically includes six cysteine residues which have been shown (in EGF) to be involved in disulphide bonds. The main structure is a two-stranded beta-sheet followed by a loop to a C-terminal short two-stranded sheet. Subdomains between the conserved cysteines vary in length. The EGF-like domain of NRG1 protein is encoded by a 3′ located part of the NRG1 gene (primarily exons 6 and 7) and is necessary for binding to ErbB-3.

The NRG1 gene and the isoforms are known under a number of different aliases such as: Neuregulin 1; Pro-NRG1; HRGA; SMDF; HGL; GGF; NDF; NRG1 Intronic Transcript 2 (Non-Protein Coding); Heregulin, Alpha (45 kD, ERBB2 P185-Activator); Acetylcholine Receptor-Inducing Activity; Pro-Neuregulin-1, Membrane-Bound Isoform; Sensory And Motor Neuron Derived Factor; Neu Differentiation Factor; Glial Growth Factor 2; NRG1-IT2; MSTP131; MST131; ARIA; GGF2; HRG1; and HRG. External Ids for NRG1 Gene are HGNC: 7997; Entrez Gene: 3084; Ensembl: ENSG00000157168; OMIM: 142445 and UniProtKB: Q02297

In an embodiment, the NRG1 polynucleotide comprises the sequence of NM_001159999.3 (SEQ ID NO: 14), which comprises exons 1-13 of NRG1. In an embodiment, the NRG1 polynucleotide consists of the sequence of NM_001159999.3 (SEQ ID NO: 14), which comprises exons 1-13 of NRG1.

In another embodiment, the NRG1 polynucleotide comprises or consists of any one or more of exons 1-13 of NRG1, recited herein as SEQ ID NOs: 1, 2, 3, 4, 5, 6, 7, 8, 9, 10, 11, 12 and 13 respectively.

Preferably the NRG1 polynucleotide comprises a sequence encoding the EGF-like domain of NRG1. Preferably the NRG1 fusion polynucleotide comprises an EGF-like domain sequence encoded by the NRG1 gene. In an embodiment this coding sequence is in-frame. In another embodiment, this coding/domain sequence is at the 3′ terminus of the NRG1 fusion polynucleotide. Preferably the method of the invention comprises determining the presence or absence of the NRG1 fusion polynucleotide in the sample by determining the presence or absence of an EGF-like domain of the NRG1 polynucleotide in the NRG1 fusion polynucleotide. The fusion is preferably in-frame. Specific methods for doing this are discussed in more detail below.

Preferably the method of the invention comprises determining the presence or absence of the NRG1 fusion polynucleotide in the sample by determining the presence or absence of a fusion junction between the NRG1 polynucleotide and the second polynucleotide in the NRG1 fusion polynucleotide and an EGF-like domain of the NRG1 polynucleotide in the NRG1 fusion polynucleotide. The fusion is preferably in-frame. Specific methods for doing this are discussed in more detail below.

In an embodiment, the NRG1 polynucleotide comprises or consists of exons 6-7 of NRG1 (SEQ ID NO: 15), exons 2-13 of NRG1 (SEQ ID NO: 16), exons 6-13 of NRG1 (SEQ ID NO: 17), or exons 6-13 of NRG1 with the additional three bases (CAT) from the 3′ end of exon 5 (SEQ ID NO: 18). Other NRG1 polynucleotides comprise or consist of exons 3-13, 4-13, 5-13, 1-12, 2-12, 3-12, 4-12, 5-12, 6-12, 1-11, 2-11, 3-11, 4-11, 5-11, 6-11, 1-10, 2-10, 3-10, 4-10, 5-10, 6-10, 1-9, 2-9, 3-9, 4-9, 5-9, 6-9, 1-8, 2-8, 3-8, 4-8, 5-8, 1-7, 2-7, 3-7, 4-7, or 5-7.

In another embodiment, the NRG1 polynucleotide comprises or consists of a fragment of at least 20 contiguous nucleotides of SEQ ID NO: 14. Preferably the fragment comprises or consists of at least about 30, at least about 40, at least about 50, at least about 60, at least about 70, at least about 80, at least about 90, at least about 100 or more contiguous nucleotides of SEQ ID NO: 14.

The NRG1 polynucleotide may comprise or consist of a variant of one of these sequences. In an embodiment, the NRG1 polynucleotide may comprise one or more (i.e. 1, 2, 3, 4, 5, 6, 7, 8, 9, 10 or more) point mutations that add, delete or substitute any of the nucleotides of SEQ ID NOs: 1, 2, 3, 4, 5, 6, 7, 8, 9, 10, 11, 12, 13, 14, 15, 16, 17 and 18. In another embodiment, the NRG1 polynucleotide has at least about 70%, at least about 80%, at least about 85%, at least about 90%, at least about 95%, at least about 97%, at least about 98% or at least about 99% sequence identity to any of SEQ ID NOs: 1, 2, 3, 4, 5, 6, 7, 8, 9, 10, 11, 12, 13, 14, 15, 16, 17 and 18. Sequence identity is typically measured over the full length of the non-variant sequence. Methods for measuring sequence identity are discussed in more detail below. In another embodiment, the NRG1 polynucleotide comprises or consists of a fragment of at least 20 contiguous nucleotides of a variant sequence having at least about 70% sequence identity or any of the other % sequence identities listed above to any of SEQ ID NOs: 1, 2, 3, 4, 5, 6, 7, 8, 9, 10, 11, 12, 13, 14, 15, 16, 17 and 18. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 1, 2, 3, 4, 5, 6, 7, 8, 9, 10, 11, 12, 13, 14, 15, 16, 17 and 18.

Also, in an embodiment, the NRG1 polynucleotide may comprise or consist of different types, such as type I, II, III of IV, or different isoforms, such as isoform A or B.

### VAPB Polynucleotide Sequences

VAPB or Vesicle-associated membrane protein-associated protein B/C is known under a number of different names such as VAMP Associated Protein B and C; VAMP (Vesicle-Associated Membrane Protein)-Associated Protein B And C; VAP-B; VAPB; ALS8; VAMP-Associated 33 KDa Protein; VAMP-Associated Protein B/C; VAMP-B/VAMP-C; VAP-B/VAP-C; VAMP-B and VAP-C. External Ids for VAPB Gene are HGNC: 12649; Entrez Gene: 9217; Ensembl: ENSG00000124164; OMIM: 605704; and UniProtKB: 095292.

In an embodiment, the VAPB polynucleotide comprises the sequence of NM_004738.4 (SEQ ID NO: 26), which comprises exons 1-6 of VAPB. In an embodiment, the VAPB polynucleotide consists of the sequence of NM_004738.4 (SEQ ID NO: 26), which comprises exons 1-6 of VAPB. In disclosed embodiments, said VAPB polynucleotide is used for detecting a VAPB-NRG1 fusion polynucleotide as further discussed herein below. In disclosed embodiments, said detecting comprises (i) the sequence of SEQ ID NO: 19, (ii) a sequence having at least about 70% sequence identity to SEQ ID NO: 19, or (iii) a fragment comprising at least about 20 contiguous nucleotides of (i) or (ii), including at least the nucleotides at positions 43 and 44 of SEQ ID NO: 19.

In another embodiment, the VAPB polynucleotide comprises or consists of any one or more of exons 1-6 of VAPB, recited herein as SEQ ID NOs: 20, 21, 22, 23, 24, and 25 respectively. In disclosed embodiments, the VAPB polynucleotide comprises or consists of exon 1 of VAPB (SEQ ID NO: 20). Other VAPB polynucleotides comprise or consist of exons 3-6, 4-6, 5-6, 1-5, 2-5, 3-5, 4-5, 1-4, 2-4, 3-4, 1-3, 2-3, or 1-2.

In another embodiment, the VAPB polynucleotide comprises or consists of a fragment of at least 20 contiguous nucleotides of SEQ ID NO: 20 or 26. Preferably the fragment comprises or consists of at least about 30, at least about 40, at least about 50, at least about 60, at least about 70, at least about 80, at least about 90, at least about 100 or more contiguous nucleotides of SEQ ID NO: 20 or 26.

The VAPB polynucleotide may comprise or consist of a variant of one of these sequences. In an embodiment, the VAPB polynucleotide may comprise one or more (i.e. 1, 2, 3, 4, 5, 6, 7, 8, 9, 10 or more) point mutations that add, delete or substitute any of the nucleotides of SEQ ID NOs: 20, 21, 22, 23, 24, 25, or 26. In another embodiment, the VAPB polynucleotide has at least about 70%, at least about 80%, at least about 85%, at least about 90%, at least about 95%, at least about 97%, at least about 98% or at least about 99% sequence identity to any of SEQ ID NOs: 20, 21, 22, 23, 24, 25, or 26. Sequence identity is typically measured over the full length of the non-variant sequence. Methods for measuring sequence identity are discussed in more detail below. In another embodiment, the VAPB polynucleotide comprises or consists of a fragment of at least 20 contiguous nucleotides of a variant sequence having at least about 70% sequence identity or any of the other % sequence identities listed above to any of SEQ ID NOs: 20, 21, 22, 23, 24, 25, or 26. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 20, 21, 22, 23, 24, 25, or 26.

### CADM1 Polynucleotide Sequences

CADM1 or Cell Adhesion Molecule 1 is known under a number of different names such as Tumor Suppressor In Lung Cancer 1; TSLC1; TSLC-1; Spermatogenic Immunoglobulin Superfamily; SgIgSF; SgIGSF; Immunoglobulin Superfamily Member 4; IGSF4; IgSF4; IGSF4A; Synaptic Cell Adhesion Molecule; SynCAM1; SYNCAM1; SynCAM; SYNCAM; Nectin-Like Protein 2; NECL2; NECL-2; Nectin-Like 2; Necl-2; RA175; ST17; BL2; Immunoglobulin Superfamily, Member 4D Variant 1; Immunoglobulin Superfamily, Member 4D Variant 2; Immunoglobulin Superfamily, Member 4; TSLC1/Nectin-Like 2/IGSF4; Truncated CADM1 Protein and STSLC-1. External Ids for CADM1 are HGNC: 5951; Entrez Gene: 23705; Ensembl: ENSG00000182985; OMIM: 605686; and UniProtKB: Q9BY67.

In an embodiment, the CADM1 polynucleotide comprises the sequence of NM_001301045.1 (SEQ ID NO: 39), which comprises exons 1-11 of CADM1. In an embodiment, the CADM1 polynucleotide consists of the sequence of NM_001301045.1 (SEQ ID NO: 39), which comprises exons 1-11 of CADM1. In disclosed embodiments, said CADM1 polynucleotide is used for detecting a CADM1-NRG1 fusion polynucleotide as further discussed herein below. In disclosed embodiments, said detecting comprises (i) the sequence of SEQ ID NO: 27, (ii) a sequence having at least about 70% sequence identity to SEQ ID NO: 27, or (iii) a fragment comprising at least about 20 contiguous nucleotides of (i) or (ii), including at least the nucleotides at positions 53 and 54 of SEQ ID NO: 27.

In another embodiment, the CADM1 polynucleotide comprises or consists of any one or more of exons 1-11 of CADM1, recited herein as SEQ ID NOs: 28, 29, 30, 31, 32, 33, 34, 35, 36, 37, and 38, respectively. In disclosed embodiments, the CADM1 polynucleotide comprises or consists of exon 7 of CADM1 (SEQ ID NO: 34).

In disclosed embodiments, the CADM1 polynucleotide comprises or consists of exons 1-7 of CADM1 (SEQ ID NO: 40). Other CADM1 polynucleotides comprise or consist of exons 2-11, 3-11, 4-11, 5-11, 6-11, 7-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2.

In another embodiment, the CADM1 polynucleotide comprises or consists of a fragment of at least 20 contiguous nucleotides of SEQ ID NO: 34, 39 or 40. Preferably the fragment comprises or consists of at least about 30, at least about 40, at least about 50, at least about 60, at least about 70, at least about 80, at least about 90, at least about 100 or more contiguous nucleotides of SEQ ID NO: 34, 39 or 40.

The CADM1 polynucleotide may comprise or consist of a variant of one of these sequences. In an embodiment, the CADM1 polynucleotide may comprise one or more (i.e. 1, 2, 3, 4, 5, 6, 7, 8, 9, 10 or more) point mutations that add, delete or substitute any of the nucleotides of SEQ ID NOs: 28, 29, 30, 31, 32, 33, 34, 35, 36, 37, 38, 39, or 40. In another embodiment, the CADM1 polynucleotide has at least about 70%, at least about 80%, at least about 85%, at least about 90%, at least about 95%, at least about 97%, at least about 98% or at least about 99% sequence identity to any of SEQ ID NOs: 28, 29, 30, 31, 32, 33, 34, 35, 36, 37, 38, 39, or 40. Sequence identity is typically measured over the full length of the non-variant sequence. Methods for measuring sequence identity are discussed in more detail below. In another embodiment, the CADM1 polynucleotide comprises or consists of a fragment of at least 20 contiguous nucleotides of a variant sequence having at least about 70% sequence identity or any of the other % sequence identities listed above to any of SEQ ID NOs: 28, 29, 30, 31, 32, 33, 34, 35, 36, 37, 38, 39, or 40. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 28, 29, 30, 31, 32, 33, 34, 35, 36, 37, 38, 39, or 40.

### CD44 Polynucleotide Sequences

CD44 or is known under a number of different names such as CD44 Molecule (Indian Blood Group); Hematopoietic Cell E- And L-Selectin Ligand; GP90 Lymphocyte Homing/Adhesion Receptor; Chondroitin Sulfate Proteoglycan 8; Extracellular Matrix Receptor III; Heparan Sulfate Proteoglycan; Phagocytic Glycoprotein 1; Hyaluronate Receptor; Hermes Antigen; CD44 Antigen; ECMR-III; HUTCH-I; Epican; MDU2; MDU3; MIC4; LHR; CD44 Antigen (Homing Function And Indian Blood Group System); Homing Function And Indian Blood Group System; Cell Surface Glycoprotein CD44; Indian Blood Group Antigen; Phagocytic Glycoprotein I; Soluble CD44; CDW44; CSPG8; HCELL; CDw44; PGP-1; MC56; Pgp1; and IN. External Ids for CD44 Gene are HGNC: 1681; Entrez Gene: 960; Ensembl: ENSG00000026508; OMIM: 107269; and UniProtKB: P16070.

In an embodiment, the CD44 polynucleotide comprises the sequence of NM_000610 (SEQ ID NO: 60), which comprises exons 1-18 of CD44. In an embodiment, the CD44 polynucleotide consists of the sequence of NM_000610 (SEQ ID NO: 60), which comprises exons 1-18 of CD44. In disclosed embodiments, said CD44 polynucleotide is used for detecting a CD44-NRG1 fusion polynucleotide as further discussed herein below. In disclosed embodiments, said detecting comprises (i) the sequence of SEQ ID NO: 41, (ii) a sequence having at least about 70% sequence identity to SEQ ID NO: 41, or (iii) a fragment comprising at least about 20 contiguous nucleotides of (i) or (ii), including at least the nucleotides at positions 52 and 53 of SEQ ID NO: 41. In an alternative embodiment, said detecting comprises (i) the sequence of SEQ ID NO: 469, (ii) a sequence having at least about 70% sequence identity to SEQ ID NO: 469, or (iii) a fragment comprising at least about 20 contiguous nucleotides of (i) or (ii), including at least the nucleotides at positions 75 and 76 of SEQ ID NO: 469.

In another embodiment, the CD44 polynucleotide comprises or consists of any one or more of exons 1-18 of CD44, recited herein as SEQ ID NOs: 42, 43, 44, 45, 46, 47, 48, 49, 50, 51, 52, 53, 54, 55, 56, 57, 58 and 59 respectively. In disclosed embodiments, the CD44 polynucleotide comprises or consists of exon 5 of CD44 (SEQ ID NO: 46).

In disclosed embodiments, the CD44 polynucleotide comprises or consists of exons 1-5 of CD44 (SEQ ID NO: 61). Other CD44 polynucleotides comprise or consist of exons 1-18, 2-18, 3-18, 4-18, 5-18, 6-18, 7-18, 8-18, 9-18, 10-18, 11-18, 12-18, 13-18, 14-18, 15-18, 16-18, 17-18, 1-17, 2-17, 3-17, 4-17, 5-17, 6-17, 7-17, 8-17, 9-17, 10-17, 11-17, 12-17, 13-17, 14-17, 15-17, 16-17, 1-16, 2-16, 3-16, 4-16, 5-16, 6-16, 7-16, 8-16, 9-16, 10-16, 11-16, 12-16, 13-16, 14-16, 15-16, 1-15, 2-15, 3-15, 4-15, 5-15, 6-15, 7-15, 8-15, 9-15, 10-15, 11-15, 12-15, 13-15, 14-15, 1-14, 2-14, 3-14, 4-14, 5-14, 6-14, 7-14, 8-14, 9-14, 10-14, 11-14, 12-14, 13-14, 1-13, 2-13, 3-13, 4-13, 5-13, 6-13, 7-13, 8-13, 9-13, 10-13, 11-13, 12-13, 1-12, 2-12, 3-12, 4-12, 5-12, 6-12, 7-12, 8-12, 9-12, 10-12, 11-12, 1-11, 2-11, 3-11, 4-11, 5-11, 6-11, 7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2.

In another embodiment, the CD44 polynucleotide comprises or consists of a fragment of at least 20 contiguous nucleotides of SEQ ID NO: 46, 60 or 61. Preferably the fragment comprises or consists of at least about 30, at least about 40, at least about 50, at least about 60, at least about 70, at least about 80, at least about 90, at least about 100 or more contiguous nucleotides of SEQ ID NO: 46, 60 or 61.

The CD44 polynucleotide may comprise or consist of a variant of one of these sequences. In an embodiment, the CD44 polynucleotide may comprise one or more (i.e. 1, 2, 3, 4, 5, 6, 7, 8, 9, 10 or more) point mutations that add, delete or substitute any of the nucleotides of SEQ ID NOs: 42, 43, 44, 45, 46, 47, 48, 49, 50, 51, 52, 53, 54, 55, 56, 57, 58, 59, 60 or 61. In another embodiment, the CD44 polynucleotide has at least about 70%, at least about 80%, at least about 85%, at least about 90%, at least about 95%, at least about 97%, at least about 98% or at least about 99% sequence identity to any of SEQ ID NOs: 42, 43, 44, 45, 46, 47, 48, 49, 50, 51, 52, 53, 54, 55, 56, 57, 58, 59, 60 or 61. Sequence identity is typically measured over the full length of the non-variant sequence. Methods for measuring sequence identity are discussed in more detail below. In another embodiment, the CD44 polynucleotide comprises or consists of a fragment of at least 20 contiguous nucleotides of a variant sequence having at least about 70% sequence identity or any of the other % sequence identities listed above to any of SEQ ID NOs: 42, 43, 44, 45, 46, 47, 48, 49, 50, 51, 52, 53, 54, 55, 56, 57, 58, 59, 60 or 61. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 42, 43, 44, 45, 46, 47, 48, 49, 50, 51, 52, 53, 54, 55, 56, 57, 58, 59, 60 or 61.

### SLC3A2 Polynucleotide Sequences

SLC3A2 or Solute Carrier Family 3 Member 2 is known under a number of different names such as Lymphocyte Activation Antigen 4F2 Large Subunit; Solute Carrier Family 3 (Activators Of Dibasic And Neutral Amino Acid Transport), Member 2; Antigen Identified By Monoclonal Antibodies 4F2, TRA1.10, TROP4, And T43; Solute Carrier Family 3 (Amino Acid Transporter Heavy Chain), Member 2; 4F2 Cell-Surface Antigen Heavy Chain; CD98 Heavy Chain; 4F2HC; MDU1; Antigen Defined By Monoclonal Antibody 4F2, Heavy Chain; Antigen Defined By Monoclonal Antibody 4F2; 4F2 Heavy Chain Antigen; 4F2 Heavy Chain; CD98 Antigen; CD98HC; 4T2HC; NACAE; CD98 and 4F2. External Ids for SLC3A2 are HGNC: 11026; Entrez Gene: 6520; Ensembl: ENSG00000168003; OMIM: 158070; and UniProtKB: P08195.

In an embodiment, the SLC3A2 polynucleotide comprises the sequence of NM_001013251.3 (SEQ ID NO: 72), which comprises exons 1-9 of transcript version 6 of SLC3A2. In an embodiment, the SLC3A2 polynucleotide consists of the sequence of NM_001013251.3 (SEQ ID NO: 72), which comprises exons 1-9 of transcript version 6 of SLC3A2. In disclosed embodiments, said SLC3A2 polynucleotide is used for detecting a SLC3A2-NRG1 fusion polynucleotide as further discussed herein below. In disclosed embodiments, said detecting comprises (i) the sequence of SEQ ID NO: 62, (ii) a sequence having at least about 70% sequence identity to SEQ ID NO: 62, or (iii) a fragment comprising at least about 20 contiguous nucleotides of (i) or (ii), including at least the nucleotides at positions 53 and 54 of SEQ ID NO: 62.

In another embodiment, the SLC3A2 polynucleotide comprises or consists of any one or more of exons 1-9 of transcript version 6 of SLC3A2, recited herein as SEQ ID NOs: 63, 64, 65, 66, 67, 68, 69, 70 and 71, respectively. In disclosed embodiments, the SLC3A2 polynucleotide comprises or consists of exon 1 of transcript version 6 of SLC3A2 (SEQ ID NO: 63). In disclosed embodiments, said SLC3A2 polynucleotide is used for detecting a SLC3A2-NRG1 fusion polynucleotide as further discussed herein below. In disclosed embodiments, said detecting comprises (i) the sequence of SEQ ID NO: 225, (ii) a sequence having at least about 70% sequence identity to SEQ ID NO: 225, or (iii) a fragment comprising at least about 20 contiguous nucleotides of (i) or (ii), including at least the nucleotides at positions 93 and 94 of SEQ ID NO: 225.

In an embodiment, the SLC3A2 polynucleotide comprises or consists of exons 2-9 of transcript version 6 of SLC3A2 (SEQ ID NO: 73). Other SLC3A2 polynucleotides comprise or consist of exons 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2.

In another embodiment, the transcript version 6 of the SLC3A2 polynucleotide comprises or consists of a fragment of at least 20 contiguous nucleotides of SEQ ID NO: 63 or 72. Preferably the fragment comprises or consists of at least about 30, at least about 40, at least about 50, at least about 60, at least about 70, at least about 80, at least about 90, at least about 100 or more contiguous nucleotides of SEQ ID NO: 63 or 72.

The SLC3A2 transcript version 6 polynucleotide may comprise or consist of a variant of one of these sequences. In an embodiment, the SLC3A2 transcript version 6 polynucleotide may comprise one or more (i.e. 1, 2, 3, 4, 5, 6, 7, 8, 9, 10 or more) point mutations that add, delete or substitute any of the nucleotides of SEQ ID NOs: 63, 64, 65, 66, 67, 68, 69, 70, 71, or 72. In another embodiment, the SLC3A2 transcript version 6 polynucleotide has at least about 70%, at least about 80%, at least about 85%, at least about 90%, at least about 95%, at least about 97%, at least about 98% or at least about 99% sequence identity to any of SEQ ID NOs: 63, 64, 65, 66, 67, 68, 69, 70, 71, or 72. Sequence identity is typically measured over the full length of the non-variant sequence. Methods for measuring sequence identity are discussed in more detail below. In another embodiment, the SLC3A2 transcript version 6 polynucleotide comprises or consists of a fragment of at least 20 contiguous nucleotides of a variant sequence having at least about 70% sequence identity or any of the other % sequence identities listed above to any of SEQ ID NOs: 63, 64, 65, 66, 67, 68, 69, 70, 71, or 72. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 63, 64, 65, 66, 67, 68, 69, 70, 71, or 72.

In another embodiment, the SLC3A2 polynucleotide comprises the sequence of NM_002394.6 (SEQ ID NO: 238), which comprises exons 1-12 of transcript version 3 of SLC3A2. In an embodiment, the SLC3A2 polynucleotide consists of the sequence of NM_002394.6 (SEQ ID NO: 238), which comprises exons 1-12 of transcript version 3 of SLC3A2.

In another embodiment, the SLC3A2 polynucleotide comprises or consists of any one or more of exons 1-12 of transcript version 3 of SLC3A2, recited herein as SEQ ID NOs: 226, 227, 228, 229, 230, 231, 232, 233, 234, 235, 236 and 237, respectively. In disclosed embodiments, the SLC3A2 polynucleotide comprises or consists of exon 2 of transcript version 3 of SLC3A2 (SEQ ID NO:227) or preferably the SLC3A2 polynucleotide comprises or consists of exon 1-2 of transcript version 3 of SLC3A2 (SEQ ID NO:239).

In an embodiment, the SLC3A2 polynucleotide comprises or consists of exons 2-12, 3-12, 4-12, 5-12, 6-12, 7-12, 8-12, 9-12, 10-12, 11-12, 1-11, 2-11, 3-11, 4-11, 5-11, 6-11, 7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2.

In another embodiment, the SLC3A2 polynucleotide comprises or consists of a fragment of at least 20 contiguous nucleotides of SEQ ID NO: 227, 238 or 239. Preferably the fragment comprises or consists of at least about 30, at least about 40, at least about 50, at least about 60, at least about 70, at least about 80, at least about 90, at least about 100 or more contiguous nucleotides of SEQ ID NO: 227, 238 or 239.

The SLC3A2 polynucleotide may comprise or consist of a variant of one of these sequences. In an embodiment, the SLC3A2 polynucleotide may comprise one or more (i.e. 1, 2, 3, 4, 5, 6, 7, 8, 9, 10 or more) point mutations that add, delete or substitute any of the nucleotides of SEQ ID NOs: 226, 227, 228, 229, 230, 231, 232, 233, 234, 235, 236 or 237. In another embodiment, the SLC3A2 polynucleotide has at least about 70%, at least about 80%, at least about 85%, at least about 90%, at least about 95%, at least about 97%, at least about 98% or at least about 99% sequence identity to any of SEQ ID NOs: 226, 227, 228, 229, 230, 231, 232, 233, 234, 235, 236 or 237. Sequence identity is typically measured over the full length of the non-variant sequence. Methods for measuring sequence identity are discussed in more detail below. In another embodiment, the SLC3A2 polynucleotide comprises or consists of a fragment of at least 20 contiguous nucleotides of a variant sequence having at least about 70% sequence identity or any of the other % sequence identities listed above to any of SEQ ID NOs: 226, 227, 228, 229, 230, 231, 232, 233, 234, 235, 236 or 237. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 226, 227, 228, 229, 230, 231, 232, 233, 234, 235, 236 or 237.

### VTCN1 Polynucleotide Sequences

VTCN1 is known under a number of different names such as V-Set Domain Containing T Cell Activation Inhibitor 1; B7-H4; B7H4; Immune Costimulatory Protein B7-H4; B7 Superfamily Member 1; B7 Family Member, H4; B7 Homolog 4; B7h.5; B7S1; T-Cell Costimulatory Molecule B7x; Protein B7S1; FLJ22418; PRO1291; VCTN1; VTCN1 and B7X. External Ids for VTCN1 are HGNC: 28873; NCBI Entrez Gene: 79679 Ensembl: ENSG00000134258 OMIM®: 608162 and UniProtKB/Swiss-Prot: Q7Z7D3.

In an embodiment, the VTCN1 polynucleotide comprises the sequence of NM_024626.4 (SEQ ID NO: 81), which comprises exons 1-6 of VTCN1. In an embodiment, the VTCN1 polynucleotide consists of the sequence of that of NM_024626.4 (SEQ ID NO: 81), which comprises exons 1-6 of VTCN1. In disclosed embodiments, said VTCN1 polynucleotide is used for detecting a VTCN1-NRG1 fusion polynucleotide as further discussed herein below.

In disclosed embodiments, said detecting comprises (i) the sequence of SEQ ID NO: 74, (ii) a sequence having at least about 70% sequence identity to SEQ ID NO: 74, or (iii) a fragment comprising at least about 20 contiguous nucleotides of (i) or (ii), including at least the nucleotides at positions 65 and 66 of SEQ ID NO: 74.

In another embodiment, the VTCN1 polynucleotide comprises or consists of any one or more of exons 1-6 of VTCN1, recited herein as SEQ ID NOs: 75, 76, 77, 78, 79 and 80, respectively. In disclosed embodiments, the VTCN1 polynucleotide comprises or consists of exon 2 of VTCN1 (SEQ ID NO: 76).

In disclosed embodiments, the VTCN1 polynucleotide comprises or consists of exons 1-2 of VTCN1 (SEQ ID NO: 82). Other VTCN1 polynucleotides comprise or consist of exons 2-6, 4-6, 5-6, 1-5, 2-5, 3-5, 4-5, 1-4, 2-4, 3-4, 1-3 or 2-3.

In another embodiment, the VTCN1 polynucleotide comprises or consists of a fragment of at least 20 contiguous nucleotides of SEQ ID NO: 76, 81 or 82. Preferably the fragment comprises or consists of at least about 30, at least about 40, at least about 50, at least about 60, at least about 70, at least about 80, at least about 90, at least about 100 or more contiguous nucleotides of SEQ ID NO: 76, 81 or 82.

The VTCN1 polynucleotide may comprise or consist of a variant of one of these sequences. In an embodiment, the VTCN1 polynucleotide may comprise one or more (i.e. 1, 2, 3, 4, 5, 6, 7, 8, 9, 10 or more) point mutations that add, delete or substitute any of the nucleotides of SEQ ID NOs: 75, 76, 77, 78, 79, 80, 81 or 82. In another embodiment, the VTCN1 polynucleotide has at least about 70%, at least about 80%, at least about 85%, at least about 90%, at least about 95%, at least about 97%, at least about 98% or at least about 99% sequence identity to any of SEQ ID NOs: 75, 76, 77, 78, 79, 80, 81 or 82. Sequence identity is typically measured over the full length of the non-variant sequence. Methods for measuring sequence identity are discussed in more detail below. In another embodiment, the VTCN1 polynucleotide comprises or consists of a fragment of at least 20 contiguous nucleotides of a variant sequence having at least about 70% sequence identity or any of the other % sequence identities listed above to any of SEQ ID NOs: 75, 76, 77, 78, 79, 80, 81 or 82. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 75, 76, 77, 78, 79, 80, 81 or 82.

### CDH1 Polynucleotide Sequences

CDH1 or Cadherin 1 is known under a number of different names such as Uvomorulin; Cadherin 1, Type 1, E-Cadherin (Epithelial); Epithelial Cadherin; E-Cadherin; Cadherin-1; CAM 120/80; CD324; CDHE; UVO; Calcium-Dependent Adhesion Protein, Epithelial; Epididymis Secretory Sperm Binding Protein; Cadherin 1, E-Cadherin (Epithelial); Cell-CAM 120/80; CD324 Antigen; E-Cadherin; Arc-1; BCDS1; ECAD; and LCAM. External Ids for CDH1 are HGNC: 1748; NCBI Entrez Gene: 999; Ensembl: ENSG00000039068; OMIM®: 192090 and UniProtKB/Swiss-Prot: P12830.

In an embodiment, the CDH1 polynucleotide comprises the sequence of NM_001317185.2 (SEQ ID NO: 100), which comprises exons 1-16 of CDH1. In an embodiment, the CDH1 polynucleotide consists of the sequence of NM_001317185.2 (SEQ ID NO: 100), which comprises exons 1-16 of CDH1. In a disclosed embodiment, the CDH1 polynucleotide comprises or consists of exon 11 of CDH1 (SEQ ID NO: 94). In disclosed embodiments, said CDH1 polynucleotide is used for detecting a CDH1-NRG1 fusion polynucleotide as further discussed herein below. In disclosed embodiments, said detecting comprises (i) the sequence of SEQ ID NO: 83, (ii) a sequence having at least about 70% sequence identity to SEQ ID NO: 83, or (iii) a fragment comprising at least about 20 contiguous nucleotides of (i) or (ii), including at least the nucleotides at positions 119 and 120 of SEQ ID NO: 83.

In another embodiment, the CDH1 polynucleotide comprises or consists of any one or more of exons 1-16 of CDH1, recited herein as SEQ ID NOs: 84, 85, 86, 87, 88, 89, 90, 91, 92, 93, 94, 95, 96, 97, 98, and 99, respectively.

In disclosed embodiments, the CDH1 polynucleotide comprises or consists of exons 1-11 of CDH1 (SEQ ID NO: 101). Other CDH1 polynucleotides comprise or consist of exons 2-16, 3-16, 4-16, 5-16, 6-16, 7-16, 8-16, 9-16, 10-16, 11-16, 12-16, 13-16, 14-16, 15-16, 1-15, 2-15, 3-15, 4-15, 5-15, 6-15, 7-15, 8-15, 9-15, 10-15, 11-15, 12-15, 13-15, 14-15, 1-14, 2-14, 3-14, 4-14, 5-14, 6-14, 7-14, 8-14, 9-14, 10-14, 11-14, 12-14, 13-14, 1-13, 2-13, 3-13, 4-13, 5-13, 6-13, 7-13, 8-13, 9-13, 10-13, 11-13, 12-13, 1-12, 2-12, 3-12, 4-12, 5-12, 6-12, 7-12, 8-12, 9-12, 10-12, 11-12, 2-11, 3-11, 4-11, 5-11, 6-11, 7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2.

In another embodiment, the CDH1 polynucleotide comprises or consists of a fragment of at least 20 contiguous nucleotides of SEQ ID NO: 94, 100 or 101. Preferably the fragment comprises or consists of at least about 30, at least about 40, at least about 50, at least about 60, at least about 70, at least about 80, at least about 90, at least about 100 or more contiguous nucleotides of SEQ ID NO: 94, 100 or 101.

The CDH1 polynucleotide may comprise or consist of a variant of one of these sequences. In an embodiment, the CDH1 polynucleotide may comprise one or more (i.e. 1, 2, 3, 4, 5, 6, 7, 8, 9, 10 or more) point mutations that add, delete or substitute any of the nucleotides of SEQ ID NOs: 84, 85, 86, 87, 88, 89, 90, 91, 92, 93, 94, 95, 96, 97, 98, 99, 100 or 101. In another embodiment, the CDH1 polynucleotide has at least about 70%, at least about 80%, at least about 85%, at least about 90%, at least about 95%, at least about 97%, at least about 98% or at least about 99% sequence identity to any of SEQ ID NOs: 84, 85, 86, 87, 88, 89, 90, 91, 92, 93, 94, 95, 96, 97, 98, 99, 100 or 101. Sequence identity is typically measured over the full length of the non-variant sequence. Methods for measuring sequence identity are discussed in more detail below. In another embodiment, the CDH1 polynucleotide comprises or consists of a fragment of at least 20 contiguous nucleotides of a variant sequence having at least about 70% sequence identity or any of the other % sequence identities listed above to any of SEQ ID NOs: 84, 85, 86, 87, 88, 89, 90, 91, 92, 93, 94, 95, 96, 97, 98, 99, 100 or 101. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 84, 85, 86, 87, 88, 89, 90, 91, 92, 93, 94, 95, 96, 97, 98, 99, 100 or 101.

### CXADR Polynucleotide Sequences

CXADR, or coxsackie virus and adenovirus receptor, is a type I membrane receptor for group B coxsackieviruses and subgroup C adenoviruses. External Ids for CXADR include HGNC: 2559 NCBI Entrez Gene: 1525 Ensembl: ENSG00000154639 OMIM®: 602621

### UniProtKB/Swiss-Prot: P78310.

In an embodiment, the CXADR polynucleotide comprises the sequence of NM_001207063.2 (SEQ ID NO: 108), which comprises exons 1-5 of CXADR. In an embodiment, the CXADR polynucleotide consists of the sequence of NM_001207063.2 (SEQ ID NO: 108), which comprises exons 1-5 of CXADR. In disclosed embodiments, said CXADR polynucleotide is used for detecting a CXADR-NRG1 fusion polynucleotide as further discussed herein below. In disclosed embodiments, said detecting comprises (i) the sequence of SEQ ID NO: 102, (ii) a sequence having at least about 70% sequence identity to SEQ ID NO: 102, or (iii) a fragment comprising at least about 20 contiguous nucleotides of (i) or (ii), including at least the nucleotides at positions 43 and 44 of SEQ ID NO: 102.

In another embodiment, the CXADR polynucleotide comprises or consists of any one or more of exons 1-5 of CXADR, recited herein as SEQ ID NOs: 103, 104, 105, 106 and 107, respectively. In a disclosed embodiment, the CXADR polynucleotide comprises or consists of exon 1 of CXADR (SEQ ID NO: 103).

In an embodiment, the CXADR polynucleotide comprises or consists of exons 1-5 of CXADR (SEQ ID NO: 108). Other CXADR polynucleotides comprise or consist of exons 2-5, 3-5, 4-5, 1-4, 2-4, 3-4, 1-3, 2-3, or 1-2.

In another embodiment, the CXADR polynucleotide comprises or consists of a fragment of at least 20 contiguous nucleotides of SEQ ID NO: 103 or 108. Preferably the fragment comprises or consists of at least about 30, at least about 40, at least about 50, at least about 60, at least about 70, at least about 80, at least about 90, at least about 100 or more contiguous nucleotides of SEQ ID NO: 103 or 108.

The CXADR polynucleotide may comprise or consist of a variant of one of these sequences. In an embodiment, the CXADR polynucleotide may comprise one or more (i.e. 1, 2, 3, 4, 5, 6, 7, 8, 9, 10 or more) point mutations that add, delete or substitute any of the nucleotides of SEQ ID NOS: 103, 104, 105, 106, 107 or 108. In another embodiment, the CXADR polynucleotide has at least about 70%, at least about 80%, at least about 85%, at least about 90%, at least about 95%, at least about 97%, at least about 98% or at least about 99% sequence identity to any of SEQ ID NOs: 103, 104, 105, 106, 107 or 108. Sequence identity is typically measured over the full length of the non-variant sequence. Methods for measuring sequence identity are discussed in more detail below. In another embodiment, the CXADR polynucleotide comprises or consists of a fragment of at least 20 contiguous nucleotides of a variant sequence having at least about 70% sequence identity or any of the other % sequence identities listed above to any of SEQ ID NOs: 103, 104, 105, 106, 107 or 108. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 103, 104, 105, 106, 107 or 108.

### GTF2E2 Polynucleotide Sequences

GTF2E2 is also known as general transcription factor IIE (TFIIE). External Ids include HGNC: 4651 NCBI Entrez Gene: 2961 Ensembl: ENSG00000197265 OMIM®: 189964 UniProtKB/Swiss-Prot: P29084.

In an embodiment, the GTF2E2 polynucleotide comprises the sequence of NM_002095.6 (SEQ ID NO: 118), which comprises exons 1-8 of GTF2E2. In an embodiment, the GTF2E2 polynucleotide consists of the sequence of NM_002095.6 (SEQ ID NO: 118), which comprises exons 1-8 of GTF2E2. In disclosed embodiments, said GTF2E2 polynucleotide is used for detecting a GTF2E2-NRG1 fusion polynucleotide as further discussed herein below. In disclosed embodiments, said detecting comprises (i) the sequence of SEQ ID NO: 109, (ii) a sequence having at least about 70% sequence identity to SEQ ID NO: 109, or (iii) a fragment comprising at least about 20 contiguous nucleotides of (i) or (ii), including at least the nucleotides at positions 141 and 142 of SEQ ID NO: 109.

In another embodiment, the GTF2E2 polynucleotide comprises or consists of any one or more of exons 1-8 of GTF2E2, recited herein as SEQ ID NOs: 110, 111, 112, 113, 114, 115, 116 and 117, respectively. In a disclosed embodiment, the VTCN1 polynucleotide comprises or consists of exon 2 of GTF2E2 (SEQ ID NO: 111).

In disclosed embodiments, the GTF2E2 polynucleotide comprises or consists of exons 1-2 (SEQ ID NO: 119) or 1-8 of GTF2E2 (SEQ ID NO: 118). Other GTF2E2 polynucleotides comprise or consist of exons 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3 or 2-3.

In another embodiment, the GTF2E2 polynucleotide comprises or consists of a fragment of at least 20 contiguous nucleotides of SEQ ID NO: 111, 118 or 119. Preferably the fragment comprises or consists of at least about 30, at least about 40, at least about 50, at least about 60, at least about 70, at least about 80, at least about 90, at least about 100 or more contiguous nucleotides of SEQ ID NO: 111, 118 or 119.

The GTF2E2 polynucleotide may comprise or consist of a variant of one of these sequences. In an embodiment, the GTF2E2 polynucleotide may comprise one or more (i.e. 1, 2, 3, 4, 5, 6, 7, 8, 9, 10 or more) point mutations that add, delete or substitute any of the nucleotides of SEQ ID NOs: 110, 111, 112, 113, 114, 115, 116, 117, 118 or 119. In another embodiment, the GTF2E2 polynucleotide has at least about 70%, at least about 80%, at least about 85%, at least about 90%, at least about 95%, at least about 97%, at least about 98% or at least about 99% sequence identity to any of SEQ ID NOs: 110, 111, 112, 113, 114, 115, 116, 117, 118 or 119. Sequence identity is typically measured over the full length of the non-variant sequence. Methods for measuring sequence identity are discussed in more detail below. In another embodiment, the GTF2E2 polynucleotide comprises or consists of a fragment of at least 20 contiguous nucleotides of a variant sequence having at least about 70% sequence identity or any of the other % sequence identities listed above to any of SEQ ID NOs: 110, 111, 112, 113, 114, 115, 116, 117, 118 or 119. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 110, 111, 112, 113, 114, 115, 116, 117, 118 or 119.

### CSMD1 Polynucleotide Sequences

CSMD1 is also known as CUB And Sushi Multiple Domains 1. External Ids for CSMD1 include HGNC: 14026 NCBI Entrez Gene: 64478 Ensembl: ENSG00000183117 OMIM®: 608397 UniProtKB/Swiss-Prot: Q96PZ7.

In an embodiment, the CSMD1 polynucleotide comprises the sequence of NM_033225.6 (SEQ ID NO: 144), which comprises exons 1-70 of CSMD1. In an embodiment, the CSMD1 polynucleotide consists of the sequence of NM_033225.6 (SEQ ID NO: 144), which comprises exons 1-70 of CSMD1. In disclosed embodiments, said CSMD1 polynucleotide is used for detecting a CSMD1-NRG1 fusion polynucleotide as further discussed herein below. In disclosed embodiments, said detecting comprises (i) the sequence of SEQ ID NO: 120, (ii) a sequence having at least about 70% sequence identity to SEQ ID NO: 120, or (iii) a fragment comprising at least about 20 contiguous nucleotides of (i) or (ii), including at least the nucleotides at positions 88 and 89 of SEQ ID NO: 120.

In another disclosed embodiment, the CSMD1 polynucleotide comprises or consists of any one or more of exons 1-23 of CSMD1, recited herein as SEQ ID NOs: 121, 122, 123, 124, 125, 126, 127, 128, 129, 130, 131, 132, 133, 134, 135, 136, 137, 138, 139, 140, 141, 142 and 143, respectively. In a disclosed embodiment, the CSMD1 polynucleotide comprises or consists of exon 23 of CSMD1 (SEQ ID NO: 143).

In an embodiment, the CSMD1 polynucleotide comprises or consists of exons 1-70 of CSMD1 (SEQ ID NO: 144) or preferably exons 1-23 (SEQ ID NO: 145). Other CSMD1 polynucleotides comprise or consist of exons 2-23, 3-23, 4-23, 5-23, 6-23, 7-23, 8-23, 9-23, 10-23, 11-23, 12-23, 13-23, 14-23, 15-23, 16-23, 17-23, 18-23, 19-23, 20-23, 21-23, 22-23, 1-22, 2-22, 3-22, 4-22, 5-22, 6-22, 7-22, 8-22, 9-22, 10-22, 11-22, 12-22, 13-22, 14-22, 15-22, 16-22, 17-22, 18-22, 19-22, 20-22, 21-22, 1-21, 2-21, 3-21, 4-21, 5-21, 6-21, 7-21, 8-21, 9-21, 10-21, 11-21, 12-21, 13-21, 14-21, 15-21, 16-21, 17-21, 18-21, 19-21, 20-21, 1-20, 2-20, 3-20, 4-20, 5-20, 6-20, 7-20, 8-20, 9-20, 10-20, 11-20, 12-20, 13-20, 14-20, 15-20, 16-20, 17-20, 18-20, 19-20, 1-19, 2-19, 3-19, 4-19, 5-19, 6-19, 7-19, 8-19, 9-19, 10-19, 11-19, 12-19, 13-19, 14-19, 15-19, 16-19, 17-19, 18-19, 1-18, 2-18, 3-18, 4-18, 5-18, 6-18, 7-18, 8-18, 9-18, 10-18, 11-18, 12-18, 13-18, 14-18, 15-18, 16-18, 17-18, 1-17, 2-17, 3-17, 4-17, 5-17, 6-17, 7-17, 8-17, 9-17, 10-17, 11-17, 12-17, 13-17, 14-17, 15-17, 16-17, 1-16, 2-16, 3-16, 4-16, 5-16, 6-16, 7-16, 8-16, 9-16, 10-16, 11-16, 12-16, 13-16, 14-16, 15-16, 1-15, 2-15, 3-15, 4-15, 5-15, 6-15, 7-15, 8-15, 9-15, 10-15, 11-15, 12-15, 13-15, 14-15, 1-14, 2-14, 3-14, 4-14, 5-14, 6-14, 7-14, 8-14, 9-14, 10-14, 11-14, 12-14, 13-14, 1-13, 2-13, 3-13, 4-13, 5-13, 6-13, 7-13, 8-13, 9-13, 10-13, 11-13, 12-13, 1-12, 2-12, 3-12, 4-12, 5-12, 6-12, 7-12, 8-12, 9-12, 10-12, 11-12, 1-11, 2-11, 3-11, 4-11, 5-11, 6-11, 7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2.

In another embodiment, the CSMD1 polynucleotide comprises or consists of a fragment of at least 20 contiguous nucleotides of SEQ ID NO: 143, 144 or 145. Preferably the fragment comprises or consists of at least about 30, at least about 40, at least about 50, at least about 60, at least about 70, at least about 80, at least about 90, at least about 100 or more contiguous nucleotides of SEQ ID NO: 143, 144 or 145.

The CSMD1 polynucleotide may comprise or consist of a variant of one of these sequences. In an embodiment, the CSMD1 polynucleotide may comprise one or more (i.e. 1, 2, 3, 4, 5, 6, 7, 8, 9, 10 or more) point mutations that add, delete or substitute any of the nucleotides of SEQ ID NOs: 121, 122, 123, 124, 125, 126, 127, 128, 129, 130, 131, 132, 133, 134, 135, 136, 137, 138, 139, 140, 141, 142, 143, 144 or 145. In another embodiment, the CSMD1 polynucleotide has at least about 70%, at least about 80%, at least about 85%, at least about 90%, at least about 95%, at least about 97%, at least about 98% or at least about 99% sequence identity to any of SEQ ID NOS: 121, 122, 123, 124, 125, 126, 127, 128, 129, 130, 131, 132, 133, 134, 135, 136, 137, 138, 139, 140, 141, 142, 143, 144 or 145. Sequence identity is typically measured over the full length of the non-variant sequence. Methods for measuring sequence identity are discussed in more detail below. In another embodiment, the CSMD1 polynucleotide comprises or consists of a fragment of at least 20 contiguous nucleotides of a variant sequence having at least about 70% sequence identity or any of the other % sequence identities listed above to any of SEQ ID NOS: 121, 122, 123, 124, 125, 126, 127, 128, 129, 130, 131, 132, 133, 134, 135, 136, 137, 138, 139, 140, 141, 142, 143, 144 or 145. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 121, 122, 123, 124, 125, 126, 127, 128, 129, 130, 131, 132, 133, 134, 135, 136, 137, 138, 139, 140, 141, 142, 143, 144 or 145.

### PTN Polynucleotide Sequences

PTN is also known as Pleiotrophin. External Ids for PTN include HGNC: 9630 NCBI Entrez Gene: 5764 Ensembl: ENSG00000105894 OMIM®: 162095 UniProtKB/Swiss-Prot: P21246.

In an embodiment, the PTN polynucleotide comprises the sequence of NM_001321386.2 (SEQ ID NO: 152), which comprises exons 1-5 of PTN. In an embodiment, the PTN polynucleotide consists of the sequence of NM_001321386.2 (SEQ ID NO: 152), which comprises exons 1-5 of PTN. In disclosed embodiments, said PTN polynucleotide is used for detecting a PTN-NRG1 fusion polynucleotide as further discussed herein below. In disclosed embodiments, said detecting comprises (i) the sequence of SEQ ID NO: 146, (ii) a sequence having at least about 70% sequence identity to SEQ ID NO: 146, or (iii) a fragment comprising at least about 20 contiguous nucleotides of (i) or (ii), including at least the nucleotides at positions 102 and 103 of SEQ ID NO: 146.

In another embodiment, the PTN polynucleotide comprises or consists of any one or more of exons 1-5 of PTN, recited herein as SEQ ID NOs: 147, 148, 149, 150 and 151, respectively. In a disclosed embodiment, the PTN1 polynucleotide comprises or consists of exon 4 of PTN1 (SEQ ID NO: 150).

In an embodiment, the PTN polynucleotide comprises or consists of exons 1-5 of PTN (SEQ ID NO: 152) or preferably exons 1-4 (SEQ ID NO: 153). Other PTN polynucleotides comprise or consist of exons 2-5, 3-5, 4-5, 2-4, 3-4, 1-3, 2-3, or 1-2.

In another embodiment, the PTN polynucleotide comprises or consists of a fragment of at least 20 contiguous nucleotides of SEQ ID NO: 150, 152 or 153. Preferably the fragment comprises or consists of at least about 30, at least about 40, at least about 50, at least about 60, at least about 70, at least about 80, at least about 90, at least about 100 or more contiguous nucleotides of SEQ ID NO: 150, 152 or 153.

The PTN polynucleotide may comprise or consist of a variant of one of these sequences. In an embodiment, the PTN polynucleotide may comprise one or more (i.e. 1, 2, 3, 4, 5, 6, 7, 8, 9, 10 or more) point mutations that add, delete or substitute any of the nucleotides of SEQ ID NOS: 147, 148, 149, 150, 151, 152 or 153. In another embodiment, the PTN polynucleotide has at least about 70%, at least about 80%, at least about 85%, at least about 90%, at least about 95%, at least about 97%, at least about 98% or at least about 99% sequence identity to any of SEQ ID NOs: 147, 148, 149, 150, 151, 152 or 153. Sequence identity is typically measured over the full length of the non-variant sequence. Methods for measuring sequence identity are discussed in more detail below. In another embodiment, the PTN polynucleotide comprises or consists of a fragment of at least 20 contiguous nucleotides of a variant sequence having at least about 70% sequence identity or any of the other % sequence identities listed above to any of SEQ ID NOs: 147, 148, 149, 150, 151, 152 or 153. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 147, 148, 149, 150, 151, 152 or 153.

### ST14 Polynucleotide Sequences

ST14 is also known as ST14 Transmembrane Serine Protease Matriptase. External Ids for ST14 include HGNC: 11344 NCBI Entrez Gene: 6768 Ensembl: ENSG00000149418 OMIM®: 606797 UniProtKB/Swiss-Prot: Q9Y5Y6.

In an embodiment, the ST14 polynucleotide comprises the sequence of NM_021978.4 (SEQ ID NO: 174), which comprises exons 1-19 of ST14. In an embodiment, the ST14 polynucleotide consists of the sequence of NM_021978.4 (SEQ ID NO: 174), which comprises exons 1-19 of ST14. In disclosed embodiments, said ST14 polynucleotide is used for detecting a ST14-NRG1 fusion polynucleotide as further discussed herein below. In disclosed embodiments, said detecting comprises (i) the sequence of SEQ ID NO: 154, (ii) a sequence having at least about 70% sequence identity to SEQ ID NO: 154, or (iii) a fragment comprising at least about 20 contiguous nucleotides of (i) or (ii), including at least the nucleotides at positions 95 and 96 of SEQ ID NO: 154.

In another embodiment, the ST14 polynucleotide comprises or consists of any one or more of exons 1-19 of ST14, recited herein as SEQ ID NOs: 155, 156, 157, 158, 159, 160, 161, 162, 163, 164, 165, 166, 167, 168, 169, 170, 171, 172 and 173, respectively. In disclosed embodiments, the ST14 polynucleotide comprises or consists of exon 11 of ST14 (SEQ ID NO: 165).

In disclosed embodiments, the ST14 polynucleotide comprises or consists of exons 1-11 of ST14 (SEQ ID NO: 175). Other ST14 polynucleotides comprise or consist of exons 1-19, 2-19, 3-19, 4-19, 5-19, 6-19, 7-19, 8-19, 9-19, 10-19, 11-19, 12-19, 13-19, 14-19, 15-19, 16-19, 17-19, 18-19, 1-18, 2-18, 3-18, 4-18, 5-18, 6-18, 7-18, 8-18, 9-18, 10-18, 11-18, 12-18, 13-18, 14-18, 15-18, 16-18, 17-18, 1-17, 2-17, 3-17, 4-17, 5-17, 6-17, 7-17, 8-17, 9-17, 10-17, 11-17, 12-17, 13-17, 14-17, 15-17, 16-17, 1-16, 2-16, 3-16, 4-16, 5-16, 6-16, 7-16, 8-16, 9-16, 10-16, 11-16, 12-16, 13-16, 14-16, 15-16, 1-15, 2-15, 3-15, 4-15, 5-15, 6-15, 7-15, 8-15, 9-15, 10-15, 11-15, 12-15, 13-15, 14-15, 1-14, 2-14, 3-14, 4-14, 5-14, 6-14, 7-14, 8-14, 9-14, 10-14, 11-14, 12-14, 13-14, 1-13, 2-13, 3-13, 4-13, 5-13, 6-13, 7-13, 8-13, 9-13, 10-13, 11-13, 12-13, 1-12, 2-12, 3-12, 4-12, 5-12, 6-12, 7-12, 8-12, 9-12, 10-12, 11-12, 2-11, 3-11, 4-11, 5-11, 6-11, 7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2.

In another embodiment, the ST14 polynucleotide comprises or consists of a fragment of at least 20 contiguous nucleotides of SEQ ID NO: 165, 174 or 175. Preferably the fragment comprises or consists of at least about 30, at least about 40, at least about 50, at least about 60, at least about 70, at least about 80, at least about 90, at least about 100 or more contiguous nucleotides of SEQ ID NO: 165, 174 or 175.

The ST14 polynucleotide may comprise or consist of a variant of one of these sequences. In an embodiment, the ST14 polynucleotide may comprise one or more (i.e. 1, 2, 3, 4, 5, 6, 7, 8, 9, 10 or more) point mutations that add, delete or substitute any of the nucleotides of SEQ ID NOs: 155, 156, 157, 158, 159, 160, 161, 162, 163, 164, 165, 166, 167, 168, 169, 170, 171, 172, 173, 174 or 175. In another embodiment, the ST14 polynucleotide has at least about 70%, at least about 80%, at least about 85%, at least about 90%, at least about 95%, at least about 97%, at least about 98% or at least about 99% sequence identity to any of SEQ ID NOs: 155, 156, 157, 158, 159, 160, 161, 162, 163, 164, 165, 166, 167, 168, 169, 170, 171, 172, 173, 174 or 175. Sequence identity is typically measured over the full length of the non-variant sequence. Methods for measuring sequence identity are discussed in more detail below. In another embodiment, the ST14 polynucleotide comprises or consists of a fragment of at least 20 contiguous nucleotides of a variant sequence having at least about 70% sequence identity or any of the other % sequence identities listed above to any of SEQ ID NOs: 155, 156, 157, 158, 159, 160, 161, 162, 163, 164, 165, 166, 167, 168, 169, 170, 171, 172, 173, 174 or 175. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 155, 156, 157, 158, 159, 160, 161, 162, 163, 164, 165, 166, 167, 168, 169, 170, 171, 172, 173, 174 or 175.

### THBS1 Polynucleotide Sequences

THBS1 is also known as Thrombospondin 1. External Ids for THBS1 include HGNC: 11785 NCBI Entrez Gene: 7057 Ensembl: ENSG00000137801 OMIM®: 188060 UniProtKB/Swiss-Prot: P07996.

In an embodiment, the THBS1 polynucleotide comprises the sequence of NM_003246.4 (SEQ ID NO: 199), which comprises exons 1-22 of THBS1. In an embodiment, the THBS1 polynucleotide consists of the sequence of NM_003246.4 (SEQ ID NO: 199), which comprises exons 1-22 of THBS1. In disclosed embodiments, said THBS1 polynucleotide is used for detecting a THBS1-NRG1 fusion polynucleotide as further discussed herein below. In disclosed embodiments, said detecting comprises (i) the sequence of SEQ ID NO: 176, (ii) a sequence having at least about 70% sequence identity to SEQ ID NO: 176, or (iii) a fragment comprising at least about 20 contiguous nucleotides of (i) or (ii), including at least the nucleotides at positions 56 and 57 of SEQ ID NO: 176.

In another embodiment, the THBS1 polynucleotide comprises or consists of any one or more of exons 1-22 of THBS1, recited herein as SEQ ID NOs: 177, 178, 179, 180, 181, 182, 183, 184, 185, 186, 187, 188, 189, 190, 191, 192, 193, 194, 195, 196, 197 and 198, respectively. In a disclosed embodiment, the THBS1 polynucleotide comprises or consists of exon 9 of THBS1 (SEQ ID NO: 185).

In an embodiment, the THBS1 polynucleotide comprises or consists of exons 1-22 of THBS1 (SEQ ID NO: 199) or preferably exons 1-9 (SEQ ID NO: 200). Other THBS1 polynucleotides comprise or consist of exons 2-22, 3-22, 4-22, 5-22, 6-22, 7-22, 8-22, 9-22, 10-22, 11-22, 12-22, 13-22, 14-22, 15-22, 16-22, 17-22, 18-22, 19-22, 20-22, 21-22, 1-21, 2-21, 3-21, 4-21, 5-21, 6-21, 7-21, 8-21, 9-21, 10-21, 11-21, 12-21, 13-21, 14-21, 15-21, 16-21, 17-21, 18-21, 19-21, 20-21, 1-20, 2-20, 3-20, 4-20, 5-20, 6-20, 7-20, 8-20, 9-20, 10-20, 11-20, 12-20, 13-20, 14-20, 15-20, 16-20, 17-20, 18-20, 19-20, 1-19, 2-19, 3-19, 4-19, 5-19, 6-19, 7-19, 8-19, 9-19, 10-19, 11-19, 12-19, 13-19, 14-19, 15-19, 16-19, 17-19, 18-19, 1-18, 2-18, 3-18, 4-18, 5-18, 6-18, 7-18, 8-18, 9-18, 10-18, 11-18, 12-18, 13-18, 14-18, 15-18, 16-18, 17-18, 1-17, 2-17, 3-17, 4-17, 5-17, 6-17, 7-17, 8-17, 9-17, 10-17, 11-17, 12-17, 13-17, 14-17, 15-17, 16-17, 1-16, 2-16, 3-16, 4-16, 5-16, 6-16, 7-16, 8-16, 9-16, 10-16, 11-16, 12-16, 13-16, 14-16, 15-16, 1-15, 2-15, 3-15, 4-15, 5-15, 6-15, 7-15, 8-15, 9-15, 10-15, 11-15, 12-15, 13-15, 14-15, 1-14, 2-14, 3-14, 4-14, 5-14, 6-14, 7-14, 8-14, 9-14, 10-14, 11-14, 12-14, 13-14, 1-13, 2-13, 3-13, 4-13, 5-13, 6-13, 7-13, 8-13, 9-13, 10-13, 11-13, 12-13, 1-12, 2-12, 3-12, 4-12, 5-12, 6-12, 7-12, 8-12, 9-12, 10-12, 11-12, 1-11, 2-11, 3-11, 4-11, 5-11, 6-11, 7-11, 8-11, 9-11, 10-11, 1-10, 2-10, 3-10, 4-10, 5-10, 6-10, 7-10, 8-10, 9-10, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2.

In another embodiment, the THBS1 polynucleotide comprises or consists of a fragment of at least 20 contiguous nucleotides of SEQ ID NO: 198, 199 or 200. Preferably the fragment comprises or consists of at least about 30, at least about 40, at least about 50, at least about 60, at least about 70, at least about 80, at least about 90, at least about 100 or more contiguous nucleotides of SEQ ID NO: 198, 199 or 200.

The THBS1 polynucleotide may comprise or consist of a variant of one of these sequences. In an embodiment, the THBS1 polynucleotide may comprise one or more (i.e. 1, 2, 3, 4, 5, 6, 7, 8, 9, 10 or more) point mutations that add, delete or substitute any of the nucleotides of SEQ ID NOS: 177, 178, 179, 180, 181, 182, 183, 184, 185, 186, 187, 188, 189, 190, 191, 192, 193, 194, 195, 196, 197, 198, 199 or 200. In another embodiment, the THBS1 polynucleotide has at least about 70%, at least about 80%, at least about 85%, at least about 90%, at least about 95%, at least about 97%, at least about 98% or at least about 99% sequence identity to any of SEQ ID NOs: 177, 178, 179, 180, 181, 182, 183, 184, 185, 186, 187, 188, 189, 190, 191, 192, 193, 194, 195, 196, 197, 198, 199 or 200. Sequence identity is typically measured over the full length of the non-variant sequence. Methods for measuring sequence identity are discussed in more detail below. In another embodiment, the THBS1 polynucleotide comprises or consists of a fragment of at least 20 contiguous nucleotides of a variant sequence having at least about 70% sequence identity or any of the other % sequence identities listed above to any of SEQ ID NOs: 177, 178, 179, 180, 181, 182, 183, 184, 185, 186, 187, 188, 189, 190, 191, 192, 193, 194, 195, 196, 197, 198, 199 or 200. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 177, 178, 179, 180, 181, 182, 183, 184, 185, 186, 187, 188, 189, 190, 191, 192, 193, 194, 195, 196, 197, 198, 199 or 200.

### AGRN Polynucleotide Sequences

AGRN is also known as Agrin. External Ids for AGRN include HGNC: 329 NCBI Entrez Gene: 375790 Ensembl: ENSG00000188157 OMIM®: 103320 UniProtKB/Swiss-Prot: 000468.

In an embodiment, the AGRN polynucleotide comprises the sequence of NM_001305275.2 (SEQ ID NO: 215), which comprises exons 1-39 of AGRN. In an embodiment, the AGRN polynucleotide consists of the sequence of NM_001305275.2 (SEQ ID NO: 215), which comprises exons 1-39 of AGRN. In disclosed embodiments, said AGRN polynucleotide is used for detecting a AGRN-NRG1 fusion polynucleotide as further discussed herein below. In disclosed embodiments, said detecting comprises (i) the sequence of SEQ ID NO: 201, (ii) a sequence having at least about 70% sequence identity to SEQ ID NO: 201, or (iii) a fragment comprising at least about 20 contiguous nucleotides of (i) or (ii), including at least the nucleotides at positions 106 and 107 of SEQ ID NO: 201.

In another embodiment, the AGRN polynucleotide comprises or consists of any one or more of exons 1-13 of AGRN, recited herein as SEQ ID NOs: 202, 203, 204, 205, 206, 207, 208, 209, 210, 211, 212, 213 and 214, respectively. In a disclosed embodiment, the AGRN polynucleotide comprises or consists of exon 12 of AGRN (SEQ ID NO: 213).

In an embodiment, the AGRN polynucleotide comprises or consists of exons 1-39 of AGRN (SEQ ID NO: 215) or preferably exons 1-12 (SEQ ID NO: 216). Other AGRN polynucleotides comprise or consist of exons 2-13, 3-13, 4-13, 5-13, 6-13, 7-13, 8-13, 9-13, 10-13, 11-13, 12-13, 2-12, 3-12, 4-12, 5-12, 6-12, 7-12, 8-12, 9-12, 10-12, 11-12, 1-11, 2-11, 3-11, 4-11, 5-11, 6-11, 7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2.

In another embodiment, the AGRN polynucleotide comprises or consists of a fragment of at least 20 contiguous nucleotides of SEQ ID NO: 214, 215 or 216. Preferably the fragment comprises or consists of at least about 30, at least about 40, at least about 50, at least about 60, at least about 70, at least about 80, at least about 90, at least about 100 or more contiguous nucleotides of SEQ ID NO: 214, 215 or 216.

The AGRN polynucleotide may comprise or consist of a variant of one of these sequences. In an embodiment, the AGRN polynucleotide may comprise one or more (i.e. 1, 2, 3, 4, 5, 6, 7, 8, 9, 10 or more) point mutations that add, delete or substitute any of the nucleotides of SEQ ID NOs: 202, 203, 204, 205, 206, 207, 208, 209, 210, 211, 212, 213, 214, 215 or 216. In another embodiment, the AGRN polynucleotide has at least about 70%, at least about 80%, at least about 85%, at least about 90%, at least about 95%, at least about 97%, at least about 98% or at least about 99% sequence identity to any of SEQ ID NOS: 202, 203, 204, 205, 206, 207, 208, 209, 210, 211, 212, 213, 214, 215 or 216. Sequence identity is typically measured over the full length of the non-variant sequence. Methods for measuring sequence identity are discussed in more detail below. In another embodiment, the AGRN polynucleotide comprises or consists of a fragment of at least 20 contiguous nucleotides of a variant sequence having at least about 70% sequence identity or any of the other % sequence identities listed above to any of SEQ ID NOs: 202, 203, 204, 205, 206, 207, 208, 209, 210, 211, 212, 213, 214, 215 or 216. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 202, 203, 204, 205, 206, 207, 208, 209, 210, 211, 212, 213, 214, 215 or 216.

### PVALB Polynucleotide Sequences

PVALP is also known was Parvalbumin. External Ids for PVALB include HGNC: 9704 NCBI Entrez Gene: 5816 Ensembl: ENSG00000100362 OMIM®: 168890 UniProtKB/Swiss-Prot: P20472.

In an embodiment, the PVALB polynucleotide comprises the sequence of NM_002854.3 (SEQ ID NO: 223), which comprises exons 1-5 of PVALB. In an embodiment, the PVALB polynucleotide consists of the sequence of NM_002854.3 (SEQ ID NO: 223), which comprises exons 1-5 of PVALB. In disclosed embodiments, said PVALB polynucleotide is used for detecting a PVALB-NRG1 fusion polynucleotide as further discussed herein below. In disclosed embodiments, said detecting comprises (i) the sequence of SEQ ID NO: 217, (ii) a sequence having at least about 70% sequence identity to SEQ ID NO: 217, or (iii) a fragment comprising at least about 20 contiguous nucleotides of (i) or (ii), including at least the nucleotides at positions 102 and 103 of SEQ ID NO: 217.

In another embodiment, the PVALB polynucleotide comprises or consists of any one or more of exons 1-5 of PVALB, recited herein as SEQ ID NOs: 218, 219, 220, 221, and 222, respectively. In a disclosed embodiment, the PVALB polynucleotide comprises or consists of exon 4 of PVALB (SEQ ID NO: 221).

In an embodiment, the PVALB polynucleotide comprises or consists of exons 1-5 of PVALB (SEQ ID NO: 223) or preferably of exons 1-4 (SEQ ID NO: 224). Other PVALB polynucleotides comprise or consist of exons 2-5, 3-5, 4-5, 2-4, 3-4, 1-3, 2-3, or 1-2.

In another embodiment, the PVALB polynucleotide comprises or consists of a fragment of at least 20 contiguous nucleotides of SEQ ID NO: 221, 223 or 224. Preferably the fragment comprises or consists of at least about 30, at least about 40, at least about 50, at least about 60, at least about 70, at least about 80, at least about 90, at least about 100 or more contiguous nucleotides of SEQ ID NO: 221, 223 or 224.

The PVALB polynucleotide may comprise or consist of a variant of one of these sequences. In an embodiment, the PVALB polynucleotide may comprise one or more (i.e. 1, 2, 3, 4, 5, 6, 7, 8, 9, 10 or more) point mutations that add, delete or substitute any of the nucleotides of SEQ ID NOs: 218, 219, 220, 221, 222, 223 or 224. In another embodiment, the PVALB polynucleotide has at least about 70%, at least about 80%, at least about 85%, at least about 90%, at least about 95%, at least about 97%, at least about 98% or at least about 99% sequence identity to any of SEQ ID NOs: 218, 219, 220, 221, 222, 223 or 224. Sequence identity is typically measured over the full length of the non-variant sequence. Methods for measuring sequence identity are discussed in more detail below. In another embodiment, the PVALB polynucleotide comprises or consists of a fragment of at least 20 contiguous nucleotides of a variant sequence having at least about 70% sequence identity or any of the other % sequence identities listed above to any of SEQ ID NOs: 218, 219, 220, 221, 222, 223 or 224. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 218, 219, 220, 221, 222, 223 or 224.

### APP Polynucleotide Sequences

APP is also known as Amyloid Beta Precursor Protein, Alpha-SAPP, AD1, Alzheimer Disease Amyloid A4 Protein Homolog, Amyloid Beta (A4) Precursor Protein, Alzheimer Disease Amyloid Protein, Cerebral Vascular Amyloid Peptide, Amyloid-Beta Precursor Protein, Amyloid Precursor Protein, Peptidase Nexin-II, Protease Nexin-II, PN-II, PreA4, ABPP, APPI, CVAP, Beta-Amyloid Precursor Protein, Testicular Tissue Protein Li 2, Beta-Amyloid Peptide (1-40), Beta-Amyloid Peptide (1-42), Amyloid Beta A4 Protein, Beta-Amyloid Peptide, Alzheimer Disease, CTFgamma, ABETA, AAA, PN2, and A4. External Ids for APP Gene are HGNC: 620; Entrez Gene: 351; Ensembl: ENSG00000142192; OMIM®: 104760 and UniProtKB/Swiss-Prot: P05067.

In an embodiment, the APP polynucleotide comprises the sequence of NM_001136130.3 (SEQ ID NO: 258), which comprises exons 1-17 of APP. In an embodiment, the APP polynucleotide consists of the sequence of NM_001136130.3 (SEQ ID NO: 258), which comprises exons 1-17 of APP. In a disclosed embodiment, the APP polynucleotide comprises or consists of exon 14 of APP (SEQ ID NO: 254). In disclosed embodiments, said APP polynucleotide is used for detecting an APP-NRG1 fusion polynucleotide as further discussed herein below. In disclosed embodiments, said detecting comprises (i) the sequence of SEQ ID NO: 240, (ii) a sequence having at least about 70% sequence identity to SEQ ID NO: 240, or (iii) a fragment comprising at least about 20 contiguous nucleotides of (i) or (ii), including at least the nucleotides at positions 54 and 55 of SEQ ID NO: 240.

In another embodiment, the APP polynucleotide comprises or consists of any one or more of exons 1-17 of APP, recited herein as SEQ ID NOs: 241, 242, 243, 244, 245, 246, 247, 248, 249, 250, 251, 252, 253, 254, 255, 256 and 257, respectively. Other APP polynucleotides comprise or consist of exons 2-17, 3-17, 4-17, 5-17, 6-17, 7-17, 8-17, 9-17, 10-17, 11-17, 12-17, 13-17, 14-17, 15-17, 16-17, 1-16, 2-16, 3-16, 4-16, 5-16, 6-16, 7-16, 8-16, 9-16, 10-16, 11-16, 12-16, 13-16, 14-16, 15-16, 1-15, 2-15, 3-15, 4-15, 5-15, 6-15, 7-15, 8-15, 9-15, 10-15, 11-15, 12-15, 13-15, 14-15, 1-14, 2-14, 3-14, 4-14, 5-14, 6-14, 7-14, 8-14, 9-14, 10-14, 11-14, 12-14, 13-14, 1-13, 2-13, 3-13, 4-13, 5-13, 6-13, 7-13, 8-13, 9-13, 10-13, 11-13, 12-13, 1-12, 2-12, 3-12, 4-12, 5-12, 6-12, 7-12, 8-12, 9-12, 10-12, 11-12, 1-11, 2-11, 3-11, 4-11, 5-11, 6-11, 7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2. Preferably, the APP polynucleotide comprises or consists of exons 1-14 of APP (SEQ ID NO: 259).

In another embodiment, the APP polynucleotide comprises or consists of a fragment of at least 20 contiguous nucleotides of SEQ ID NO: 254 or 259. Preferably the fragment comprises or consists of at least about 30, at least about 40, at least about 50, at least about 60, at least about 70, at least about 80, at least about 90, at least about 100 or more contiguous nucleotides of SEQ ID NO: 254 or 259.

The APP polynucleotide may comprise or consist of a variant of one of these sequences. In an embodiment, the APP polynucleotide may comprise one or more (i.e. 1, 2, 3, 4, 5, 6, 7, 8, 9, 10 or more) point mutations that add, delete or substitute any of the nucleotides of SEQ ID NOs: 241, 242, 243, 244, 245, 246, 247, 248, 249, 250, 251, 252, 253, 254, 255, 256, 257, 258 or 259. In another embodiment, the APP polynucleotide has at least about 70%, at least about 80%, at least about 85%, at least about 90%, at least about 95%, at least about 97%, at least about 98% or at least about 99% sequence identity to any of SEQ ID NOs: 241, 242, 243, 244, 245, 246, 247, 248, 249, 250, 251, 252, 253, 254, 255, 256, 257, 258 or 259. Sequence identity is typically measured over the full length of the non-variant sequence. Methods for measuring sequence identity are discussed in more detail below. In another embodiment, the VAPB polynucleotide comprises or consists of a fragment of at least 20 contiguous nucleotides of a variant sequence having at least about 70% sequence identity or any of the other % sequence identities listed above to any of SEQ ID NOs: 241, 242, 243, 244, 245, 246, 247, 248, 249, 250, 251, 252, 253, 254, 255, 256, 257, 258 or 259. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 241, 242, 243, 244, 245, 246, 247, 248, 249, 250, 251, 252, 253, 254, 255, 256, 257, 258 or 259.

### WRN Polynucleotide Sequences

WRN or Werner Syndrome ATP-Dependent Helicase is also known as WRN RecQ Like Helicase; RECQL2; RECQ3; Werner Syndrome RecQ Like Helicase; DNA Helicase, RecQ-Like Type 3; RecQ Protein-Like 2; Exonuclease WRN; Werner Syndrome, RecQ Helicase-Like; Werner Syndrome; EC 3.6.4.12; RECQL3; and RecQ3. External Ids for WRN Gene are HGNC: 12791; NCBI Entrez Gene: 7486; Ensembl: ENSG00000165392; OMIM®: 604611; and UniProtKB/Swiss-Prot: Q14191.

In an embodiment, the WRN polynucleotide comprises the sequence of NM_000553.6 (SEQ ID NO: 296), which comprises exons 1-35 of WRN. In an embodiment, the WRN polynucleotide consists of the sequence of NM_000553.6 (SEQ ID NO: 296), which comprises exons 1-35 of WRN. In disclosed embodiments, said WRN polynucleotide is used for detecting a WRN-NRG1 fusion polynucleotide as further discussed herein below. In disclosed embodiments, said detecting comprises (i) the sequence of SEQ ID NO: 260, (ii) a sequence having at least about 70% sequence identity to SEQ ID NO: 260, or (iii) a fragment comprising at least about 20 contiguous nucleotides of (i) or (ii), including at least the nucleotides at positions 96 and 97 of SEQ ID NO: 260.

In another embodiment, the WRN polynucleotide comprises or consists of any one or more of exons 1-35 of WRN, recited herein as SEQ ID NOs: 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294 and 295, respectively. In a disclosed embodiment, the WRN polynucleotide comprises or consists of exon 33 of WRN (SEQ ID NO: 293).

In a disclosed embodiment, the WRN polynucleotide comprises or consists of exons 1-33 of WRN (SEQ ID NO:297). Other WRN polynucleotides comprise or consist of exons 2-33, 3-33, 4-33, 5-33, 6-33, 7-33, 8-33, 9-33, 10-33, 11-33, 12-33, 13-33, 14-33, 15-33, 16-33, 17-33, 18-33, 19-33, 20-33, 21-33, 22-33, 23-33, 24-33, 25-33, 26-33, 27-33, 28-33, 29-33, 30-33, 31-33, 32-33, 1-32, 2-32, 3-32, 4-32, 5-32, 6-32, 7-32, 8-32, 9-32, 10-32, 11-32, 12-32, 13-32, 14-32, 15-32, 16-32, 17-32, 18-32, 19-32, 20-32, 21-32, 22-32, 23-32, 24-32, 25-32, 26-32, 27-32, 28-32, 29-32, 30-32, 31-32, 1-31, 2-31, 3-31, 4-31, 5-31, 6-31, 7-31, 8-31, 9-31, 10-31, 11-31, 12-31, 13-31, 14-31, 15-31, 16-31, 17-31, 18-31, 19-31, 20-31, 21-31, 22-31, 23-31, 24-31, 25-, 31 26-31, 27-31, 28-31, 29-31, 30-31, 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, 29-30, 1-29, 2-29, 3-29, 4-29, 5-29, 6-29, 7-29, 8-29, 9-29, 10-29, 11-29, 12-29, 13-29, 14-29, 15-29, 16-29, 17-29, 18-29, 19-29, 20-29, 21-29, 22-29, 23-29, 24-29, 25-29, 26-29, 27-29, 28-29, 1-28, 2-28, 3-28, 4-28, 5-28, 6-28, 7-28, 8-28, 9-28, 10-28, 11-28, 12-28, 13-28, 14-28, 15-28, 16-28, 17-28, 18-28, 19-28, 20-28, 21-28, 22-28, 23-28, 24-28, 25-28, 26-28, 27-28, 1-27, 2-27, 3-27, 4-27, 5-27, 6-27, 7-27, 8-27, 9-27, 10-27, 11-27, 12-27, 13-27, 14-27, 15-27, 16-27, 17-27, 18-27, 19-27, 20-27, 21-27, 22-27, 23-27, 24-27, 25-27, 26-27, 1-26, 2-26, 3-26, 4-26, 5-26, 6-26, 7-26, 8-26, 9-26, 10-26, 11-26, 12-26, 13-26, 14-26, 15-26, 16-26, 17-26, 18-26, 19-26, 20-26, 21-26, 22-26, 23-26, 24-26, 25-26, 1-25, 2-25, 3-25, 4-25, 5-25, 6-25, 7-25, 8-25, 9-25, 10-25, 11-25, 12-25, 13-25, 14-25, 15-25, 16-25, 17-25, 18-25, 19-25, 20-25, 21-25, 22-25, 23-25, 24-25, 1-24, 2-24, 3-24, 4-24, 5-24, 6-24, 7-24, 8-24, 9-24, 10-24, 11-24, 12-24, 13-24, 14-24, 15-24, 16-24, 17-24, 18-24, 19-24, 20-24, 21-24, 22-24, 23-24, 1-23, 2-23, 3-23, 4-23, 5-23, 6-23, 7-23, 8-23, 9-23, 10-23, 11-23, 12-23, 13-23, 14-23, 15-23, 16-23, 17-23, 18-23, 19-23, 20-23, 21-23, 22-23, 1-22, 2-22, 3-22, 4-22, 5-22, 6-22, 7-22, 8-22, 9-22, 10-22, 11-22, 12-22, 13-22, 14-22, 15-22, 16-22, 17-22, 18-22, 19-22, 20-22, 21-22, 1-21, 2-21, 3-21, 4-21, 5-21, 6-21, 7-21, 8-21, 9-21, 10-21, 11-21, 12-21, 13-21, 14-21, 15-21, 16-21, 17-21, 18-21, 19-21, 20-21, 1-20, 2-20, 3-20, 4-20, 5-20, 6-20, 7-20, 8-20, 9-20, 10-20, 11-20, 12-20, 13-20, 14-20, 15-20, 16-20, 17-20, 18-20, 19-20, 1-19, 2-19, 3-19, 4-19, 5-19, 6-19, 7-19, 8-19, 9-19, 10-19, 11-19, 12-19, 13-19, 14-19, 15-19, 16-19, 17-19, 18-19, 1-18, 2-18, 3-18, 4-18, 5-18, 6-18, 7-18, 8-18, 9-18, 10-18, 11-18, 12-18, 13-18, 14-18, 15-18, 16-18, 17-18, 1-17, 2-17, 3-17, 4-17, 5-17, 6-17, 7-17, 8-17, 9-17, 10-17, 11-17, 12-17, 13-17, 14-17, 15-17, 16-17, 1-16, 2-16, 3-16, 4-16, 5-16, 6-16, 7-16, 8-16, 9-16, 10-16, 11-16, 12-16, 13-16, 14-16, 15-16, 1-15, 2-15, 3-15, 4-15, 5-15, 6-15, 7-15, 8-15, 9-15, 10-15, 11-15, 12-15, 13-15, 14-15, 1-14, 2-14, 3-14, 4-14, 5-14, 6-14, 7-14, 8-14, 9-14, 10-14, 11-14, 12-14, 13-14, 1-13, 2-13, 3-13, 4-13, 5-13, 6-13, 7-13, 8-13, 9-13, 10-13, 11-13, 12-13, 1-12, 2-12, 3-12, 4-12, 5-12, 6-12, 7-12, 8-12, 9-12, 10-12, 11-12, 1-11, 2-11, 3-11, 4-11, 5-11, 6-11, 7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2.

In another embodiment, the WRN polynucleotide comprises or consists of a fragment of at least 20 contiguous nucleotides of SEQ ID NO: 293, 296 or 297. Preferably the fragment comprises or consists of at least about 30, at least about 40, at least about 50, at least about 60, at least about 70, at least about 80, at least about 90, at least about 100 or more contiguous nucleotides of SEQ ID NO: 293, 296 or 297.

The WRN polynucleotide may comprise or consist of a variant of one of these sequences. In an embodiment, the WRN polynucleotide may comprise one or more (i.e. 1, 2, 3, 4, 5, 6, 7, 8, 9, 10 or more) point mutations that add, delete or substitute any of the nucleotides of SEQ ID NOs: 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296 or 297. In another embodiment, the WRN polynucleotide has at least about 70%, at least about 80%, at least about 85%, at least about 90%, at least about 95%, at least about 97%, at least about 98% or at least about 99% sequence identity to any of SEQ ID NOs: 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296 or 297. Sequence identity is typically measured over the full length of the non-variant sequence. Methods for measuring sequence identity are discussed in more detail below. In another embodiment, the WRN polynucleotide comprises or consists of a fragment of at least 20 contiguous nucleotides of a variant sequence having at least about 70% sequence identity or any of the other % sequence identities listed above to any of SEQ ID NOs: 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296 or 297. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296 or 297.

### DAAM1

DAAM1 or Disheveled Associated Activator Of Morphogenesis 1 is also known as KIAA0666; and Disheveled-Associated Activator Of Morphogenesis 1. External Ids for DAAM1 Gene are HGNC: 18142; NCBI Entrez Gene: 23002; Ensembl: ENSG00000100592; OMIM®: 606626; and UniProtKB/Swiss-Prot: Q9Y4D1.

In an embodiment, the DAAM1 polynucleotide comprises the sequence of NM_001270520.2 (SEQ ID NO: 324), which comprises exons 1-25 of DAAM1. In an embodiment, the DAAM1 polynucleotide consists of the sequence of NM_001270520.2 (SEQ ID NO: 324), which comprises exons 1-25 of DAAM1. In disclosed embodiments, said DAAM1 polynucleotide is used for detecting a DAAM1-NRG1 fusion polynucleotide as further discussed herein below. In disclosed embodiments, said detecting comprises (i) the sequence of SEQ ID NO: 298, (ii) a sequence having at least about 70% sequence identity to SEQ ID NO: 298, or (iii) a fragment comprising at least about 20 contiguous nucleotides of (i) or (ii), including at least the nucleotides at positions 75 and 76 of SEQ ID NO: 298.

In another embodiment, the DAAM1 polynucleotide comprises or consists of any one or more of exons 1-25 of DAAM1, recited herein as SEQ ID NOs: 299, 300, 301, 302, 303, 304, 305, 306, 307, 308, 309, 310, 311, 312, 313, 314, 315, 316, 317, 318, 319, 320, 321, 322 and 323, respectively. In a disclosed embodiment, the DAAM1 polynucleotide comprises or consists of exon 1 of DAAM1 (SEQ ID NO: 299).

In another embodiment, the DAAM1 polynucleotide comprises or consists of a fragment of at least 20 contiguous nucleotides of SEQ ID NO: 299 or 324. Preferably the fragment comprises or consists of at least about 30, at least about 40, at least about 50, at least about 60, at least about 70, at least about 80, at least about 90, at least about 100 or more contiguous nucleotides of SEQ ID NO: 299 or 324.

The DAAM1 polynucleotide may comprise or consist of a variant of one of these sequences. In an embodiment, the DAAM1 polynucleotide may comprise one or more (i.e. 1, 2, 3, 4, 5, 6, 7, 8, 9, 10 or more) point mutations that add, delete or substitute any of the nucleotides of SEQ ID NOs: 299, 300, 301, 302, 303, 304, 305, 306, 307, 308, 309, 310, 311, 312, 313, 314, 315, 316, 317, 318, 319, 320, 321, 322, 323 or 324. In another embodiment, the DAAM1 polynucleotide has at least about 70%, at least about 80%, at least about 85%, at least about 90%, at least about 95%, at least about 97%, at least about 98% or at least about 99% sequence identity to any of SEQ ID NOs: 299, 300, 301, 302, 303, 304, 305, 306, 307, 308, 309, 310, 311, 312, 313, 314, 315, 316, 317, 318, 319, 320, 321, 322, 323 or 324. Sequence identity is typically measured over the full length of the non-variant sequence. Methods for measuring sequence identity are discussed in more detail below. In another embodiment, the DAAM1 polynucleotide comprises or consists of a fragment of at least 20 contiguous nucleotides of a variant sequence having at least about 70% sequence identity or any of the other % sequence identities listed above to any of SEQ ID NOs: 299, 300, 301, 302, 303, 304, 305, 306, 307, 308, 309, 310, 311, 312, 313, 314, 315, 316, 317, 318, 319, 320, 321, 322, 323 or 324. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 299, 300, 301, 302, 303, 304, 305, 306, 307, 308, 309, 310, 311, 312, 313, 314, 315, 316, 317, 318, 319, 320, 321, 322, 323 or 324.

### ASPH

ASPH or Aspartate Beta-Hydroxylase is also known as BAH, CASQ2BP1, JCTN, HAAH, Aspartyl/Asparaginyl Beta-Hydroxylase, Peptide-Aspartate Beta-Dioxygenase, ASP Beta-Hydroxylase, Junctate, Junctin, Humbug, Cardiac Junctin, EC 1.14.11.16, A Beta H-J-J, FDLAB and AAH. External IDs for ASPH Gene are HGNC: 757; Entrez Gene: 444; Ensembl: ENSG00000198363; OMIM®: 600582 and UniProtKB/Swiss-Prot: Q12797.

In an embodiment, the ASPH polynucleotide comprises the sequence of NM_001164750.2 (SEQ ID NO: 351), which comprises exons 1-25 of ASPH. In an embodiment, the ASPH polynucleotide consists of the sequence of NM_001164750.2 (SEQ ID NO: 351), which comprises exons 1-25 of ASPH. In disclosed embodiments, said ASPH polynucleotide is used for detecting a ASPH-NRG1 fusion polynucleotide as further discussed herein below. In disclosed embodiments, said detecting comprises (i) the sequence of SEQ ID NO: 325, (ii) a sequence having at least about 70% sequence identity to SEQ ID NO: 325, or (iii) a fragment comprising at least about 20 contiguous nucleotides of (i) or (ii), including at least the nucleotides at positions 75 and 76 of SEQ ID NO: 325.

In another embodiment, the ASPH polynucleotide comprises or consists of any one or more of exons 1-25 of ASPH, recited herein as SEQ ID NOs: 326, 327, 328, 329, 330, 331, 332, 333, 334, 335, 336, 337, 338, 339, 340, 341, 342, 343, 344, 345, 346, 347, 348, 349 and 350, respectively. In a disclosed embodiment, the ASPH polynucleotide comprises or consists of exon 22 of ASPH (SEQ ID NO: 347).

In a disclosed embodiment, the ASPH polynucleotide comprises or consists of exons 1-22 of ASPH (SEQ ID NO: 352). Other ASPH polynucleotides comprise or consist of exons 1-25, 2-25, 3-25, 4-25, 5-25, 6-25, 7-25, 8-25, 9-25, 10-25, 11-25, 12-25, 13-25, 14-25, 15-25, 16-25, 17-25, 18-25, 19-25, 20-25, 21-25, 22-25, 23-25, 24-25, 1-24, 2-24, 3-24, 4-24, 5-24, 6-24, 7-24, 8-24, 9-24, 10-24, 11-24, 12-24, 13-24, 14-24, 15-24, 16-24, 17-24, 18-24, 19-24, 20-24, 21-24, 22-24, 23-24, 1-23, 2-23, 3-23, 4-23, 5-23, 6-23, 7-23, 8-23, 9-23, 10-23, 11-23, 12-23, 13-23, 14-23, 15-23, 16-23, 17-23, 18-23, 19-23, 20-23, 21-23, 22-23, 2-22, 3-22, 4-22, 5-22, 6-22, 7-22, 8-22, 9-22, 10-22, 11-22, 12-22, 13-22, 14-22, 15-22, 16-22, 17-22, 18-22, 19-22, 20-22, 21-22, 1-21, 2-21, 3-21, 4-21, 5-21, 6-21, 7-21, 8-21, 9-21, 10-21, 11-21, 12-21, 13-21, 14-21, 15-21, 16-21, 17-21, 18-21, 19-21, 20-21, 1-20, 2-20, 3-20, 4-20, 5-20, 6-20, 7-20, 8-20, 9-20, 10-20, 11-20, 12-20, 13-20, 14-20, 15-20, 16-20, 17-20, 18-20, 19-20, 1-19, 2-19, 3-19, 4-19, 5-19, 6-19, 7-19, 8-19, 9-19, 10-19, 11-19, 12-19, 13-19, 14-19, 15-19, 16-19, 17-19, 18-19, 1-18, 2-18, 3-18, 4-18, 5-18, 6-18, 7-18, 8-18, 9-18, 10-18, 11-18, 12-18, 13-18, 14-18, 15-18, 16-18, 17-18, 1-17, 2-17, 3-17, 4-17, 5-17, 6-17, 7-17, 8-17, 9-17, 10-17, 11-17, 12-17, 13-17, 14-17, 15-17, 16-17, 1-16, 2-16, 3-16, 4-16, 5-16, 6-16, 7-16, 8-16, 9-16, 10-16, 11-16, 12-16, 13-16, 14-16, 15-16, 1-15, 2-15, 3-15, 4-15, 5-15, 6-15, 7-15, 8-15, 9-15, 10-15, 11-15, 12-15, 13-15, 14-15, 1-14, 2-14, 3-14, 4-14, 5-14, 6-14, 7-14, 8-14, 9-14, 10-14, 11-14, 12-14, 13-14, 1-13, 2-13, 3-13, 4-13, 5-13, 6-13, 7-13, 8-13, 9-13, 10-13, 11-13, 12-13, 1-12, 2-12, 3-12, 4-12, 5-12, 6-12, 7-12, 8-12, 9-12, 10-12, 11-12, 1-11, 2-11, 3-11, 4-11, 5-11, 6-11, 7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2.

In another embodiment, the ASPH polynucleotide comprises or consists of a fragment of at least 20 contiguous nucleotides of SEQ ID NO: 347, 351 or 352. Preferably the fragment comprises or consists of at least about 30, at least about 40, at least about 50, at least about 60, at least about 70, at least about 80, at least about 90, at least about 100 or more contiguous nucleotides of SEQ ID NO: 347, 351 or 352.

The ASPH polynucleotide may comprise or consist of a variant of one of these sequences. In an embodiment, the ASPH polynucleotide may comprise one or more (i.e. 1, 2, 3, 4, 5, 6, 7, 8, 9, 10 or more) point mutations that add, delete or substitute any of the nucleotides of SEQ ID NOS: 326, 327, 328, 329, 330, 331, 332, 333, 334, 335, 336, 337, 338, 339, 340, 341, 342, 343, 344, 345, 346, 347, 348, 349, 350, 351 and 352. In another embodiment, the ASPH polynucleotide has at least about 70%, at least about 80%, at least about 85%, at least about 90%, at least about 95%, at least about 97%, at least about 98% or at least about 99% sequence identity to any of SEQ ID NOs: 326, 327, 328, 329, 330, 331, 332, 333, 334, 335, 336, 337, 338, 339, 340, 341, 342, 343, 344, 345, 346, 347, 348, 349, 350, 351 and 352. Sequence identity is typically measured over the full length of the non-variant sequence. Methods for measuring sequence identity are discussed in more detail below. In another embodiment, the ASPH polynucleotide comprises or consists of a fragment of at least 20 contiguous nucleotides of a variant sequence having at least about 70% sequence identity or any of the other % sequence identities listed above to any of SEQ ID NOs: 326, 327, 328, 329, 330, 331, 332, 333, 334, 335, 336, 337, 338, 339, 340, 341, 342, 343, 344, 345, 346, 347, 348, 349, 350, 351 and 352. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 326, 327, 328, 329, 330, 331, 332, 333, 334, 335, 336, 337, 338, 339, 340, 341, 342, 343, 344, 345, 346, 347, 348, 349, 350, 351 and 352.

### NOTCH2

NOTCH2 or Notch Receptor 2 is also as Notch 2; Neurogenic Locus Notch Homolog Protein 2; HN2; Notch (Drosophila) Homolog 2; Notch Homolog 2 (Drosophila); Notch Homolog 2; HJCYS; and AGS2. External Ids for NOTCH2 Gene are HGNC: 7882; NCBI Entrez Gene: 4853; Ensembl: ENSG00000134250; OMIM®: 600275; and UniProtKB/Swiss-Prot: Q04721.

In an embodiment, the NOTCH2 polynucleotide comprises the sequence of NM_024408.4 (SEQ ID NO: 362, which comprises exons 1-34 of NOTCH2. In an embodiment, the NOTCH2 polynucleotide consists of the sequence of NM_024408.4 (SEQ ID NO: 362), which comprises exons 1-34 of NOTCH2. In disclosed embodiments, said NOTCH2 polynucleotide is used for detecting a NOTCH2-NRG1 fusion polynucleotide as further discussed herein below. In disclosed embodiments, said detecting comprises (i) the sequence of SEQ ID NO: 353, (ii) a sequence having at least about 70% sequence identity to SEQ ID NO: 353, or (iii) a fragment comprising at least about 20 contiguous nucleotides of (i) or (ii), including at least the nucleotides at positions 75 and 76 of SEQ ID NO: 353.

In another embodiment, the NOTCH2 polynucleotide comprises or consists of any one or more of exons 1-34 of NOTCH2, recited herein as SEQ ID NOs: 354, 355, 356, 357, 358, 359 or 360, respectively. In a disclosed embodiment, the NOTCH2 polynucleotide comprises or consists of exon 6 of NOTCH2 (SEQ ID NO: 359).

In a disclosed embodiment, the NOTCH2 polynucleotide comprises or consists of exons 1-6 of NOTCH2 (SEQ ID NO: 363). Other NOTCH2 polynucleotides comprise or consist of exons 2-6, 3-6, 4-6, 5-6, 1-5, 2-5, 3-5, 4-5, 1-4, 2-4, 3-4, 1-3, 2-3, or 1-2.

In another embodiment, the NOTCH2 polynucleotide comprises or consists of a fragment of at least 20 contiguous nucleotides of SEQ ID NO: 359, 362 or 363. Preferably the fragment comprises or consists of at least about 30, at least about 40, at least about 50, at least about 60, at least about 70, at least about 80, at least about 90, at least about 100 or more contiguous nucleotides of SEQ ID NO: 359, 362 or 363.

The NOTCH2 polynucleotide may comprise or consist of a variant of one of these sequences. In an embodiment, the NOTCH2 polynucleotide may comprise one or more (i.e. 1, 2, 3, 4, 5, 6, 7, 8, 9, 10 or more) point mutations that add, delete or substitute any of the nucleotides of SEQ ID NOs: 354, 355, 356, 357, 358, 359, 360, 361, 362 or 363. In another embodiment, the NOTCH2 polynucleotide has at least about 70%, at least about 80%, at least about 85%, at least about 90%, at least about 95%, at least about 97%, at least about 98% or at least about 99% sequence identity to any of SEQ ID NOs: 354, 355, 356, 357, 358, 359, 360, 361, 362 or 363. Sequence identity is typically measured over the full length of the non-variant sequence. Methods for measuring sequence identity are discussed in more detail below. In another embodiment, the NOTCH2 polynucleotide comprises or consists of a fragment of at least 20 contiguous nucleotides of a variant sequence having at least about 70% sequence identity or any of the other % sequence identities listed above to any of SEQ ID NOS: 354, 355, 356, 357, 358, 359, 360, 361, 362 or 363. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 354, 355, 356, 357, 358, 359, 360, 361, 362 or 363.

### CD74

CD74 is also known as CD74 Molecule; DHLAG; CD74 Molecule, Major Histocompatibility Complex, Class II Invariant Chain; HLA Class II Histocompatibility Antigen Gamma Chain; Class II MHC-Associated Invariant Chain Peptide; HLA-DR Antigens-Associated Invariant Chain; Gamma Chain Of Class II Antigens; Ia-Associated Invariant Chain; MHC HLA-DR Gamma Chain; HLA-DR-Gamma; CLIP; Ia Antigen-Associated Invariant Chain; CD74 Antigen; Ia-GAMMA; HLADG; P33; II; and Ii. External Ids for CD74 Gene are HGNC: 1697; NCBI Entrez Gene: 972; Ensembl: ENSG00000019582; OMIM®: 142790; and UniProtKB/Swiss-Prot: P04233.

In an embodiment, the CD74 polynucleotide comprises the sequence of NM_001025159.3 (SEQ ID NO: 374), which comprises exons 1-9 of CD74. In an embodiment, the CD74 polynucleotide consists of the sequence of NM_001025159.3 (SEQ ID NO: 374), which comprises exons 1-9 of CD74. In disclosed embodiments, said CD74 polynucleotide is used for detecting a CD74-NRG1 fusion polynucleotide as further discussed herein below. In disclosed embodiments, said detecting comprises (i) the sequence of SEQ ID NO: 364, (ii) a sequence having at least about 70% sequence identity to SEQ ID NO: 364, or (iii) a fragment comprising at least about 20 contiguous nucleotides of (i) or (ii), including at least the nucleotides at positions 75 and 76 of SEQ ID NO: 364.

In another embodiment, the CD74 polynucleotide comprises or consists of any one or more of exons 1-9 of CD74, recited herein as SEQ ID NOs: 365, 366, 367, 368, 369, 370, 371, 372 and 373, respectively. In a disclosed embodiment, the CD74 polynucleotide comprises or consists of exon 2 of CD74 (SEQ ID NO: 366).

In a disclosed embodiment, the CD74 polynucleotide comprises or consists of exons 1-2 of CD74 (SEQ ID NO: 375).

In another embodiment, the CD74 polynucleotide comprises or consists of a fragment of at least 20 contiguous nucleotides of SEQ ID NO: 366, 374 or 375. Preferably the fragment comprises or consists of at least about 30, at least about 40, at least about 50, at least about 60, at least about 70, at least about 80, at least about 90, at least about 100 or more contiguous nucleotides of SEQ ID NO: 366, 374 or 375.

The CD74 polynucleotide may comprise or consist of a variant of one of these sequences. In an embodiment, the CD74 polynucleotide may comprise one or more (i.e. 1, 2, 3, 4, 5, 6, 7, 8, 9, 10 or more) point mutations that add, delete or substitute any of the nucleotides of SEQ ID NOs: 365, 366, 367, 368, 369, 370, 371, 372, 373, 374 or 375. In another embodiment, the CD74 polynucleotide has at least about 70%, at least about 80%, at least about 85%, at least about 90%, at least about 95%, at least about 97%, at least about 98% or at least about 99% sequence identity to any of SEQ ID NOs: 365, 366, 367, 368, 369, 370, 371, 372, 373, 374 or 375. Sequence identity is typically measured over the full length of the non-variant sequence. Methods for measuring sequence identity are discussed in more detail below. In another embodiment, the CD74 polynucleotide comprises or consists of a fragment of at least 20 contiguous nucleotides of a variant sequence having at least about 70% sequence identity or any of the other % sequence identities listed above to any of SEQ ID NOS: 365, 366, 367, 368, 369, 370, 371, 372, 373, 374 or 375. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 365, 366, 367, 368, 369, 370, 371, 372, 373, 374 or 375.

### SDC4

SDC4 or Syndecan 4 is also known as Amphiglycan; SYND4; Syndecan 4 (Amphiglycan, Ryudocan); Syndecan Proteoglycan 4; Ryudocan Core Protein; Syndecan-4; Ryudocan; and Ryudocan Amphiglycan. External Ids for SDC4 Gene are HGNC: 10661; NCBI Entrez Gene: 6385; Ensembl: ENSG00000124145; OMIM®: 600017; and UniProtKB/Swiss-Prot: P31431.

In an embodiment, the SDC4 polynucleotide comprises the sequence of NM_002999.4 (SEQ ID NO: 382), which comprises exons 1-5 of SDC4. In an embodiment, the SDC4 polynucleotide consists of the sequence of NM_002999.4 (SEQ ID NO: 382), which comprises exons 1-5 of SDC4. In disclosed embodiments, the SDC4 polynucleotide is used for detecting a SDC4-NRG1 fusion polynucleotide as further discussed herein below. In disclosed embodiments, said detecting comprises (i) the sequence of SEQ ID NO: 376, (ii) a sequence having at least about 70% sequence identity to SEQ ID NO: 376, or (iii) a fragment comprising at least about 20 contiguous nucleotides of (i) or (ii), including at least the nucleotides at positions 75 and 76 of SEQ ID NO: 376. In an alternative embodiment, said detecting comprises (i) the sequence of SEQ ID NO: 468, (ii) a sequence having at least about 70% sequence identity to SEQ ID NO: 468, or (iii) a fragment comprising at least about 20 contiguous nucleotides of (i) or (ii), including at least the nucleotides at positions 75 and 76 of SEQ ID NO: 468.

In another embodiment, the SDC4 polynucleotide comprises or consists of any one or more of exons 1-5 of SDC4, recited herein as SEQ ID NOs: 377, 378, 379, 380 and 381, respectively. In a disclosed embodiment, the SDC4 polynucleotide comprises or consists of exon 2 of SDC4 (SEQ ID NO: 378) or comprises or consists of exon 4 of SDC4 (SEQ ID NO: 380).

In a disclosed embodiment, the SDC4 polynucleotide comprises or consists of exons 1-2 of SDC4 (SEQ ID NO: 383) or exons 1-4 of SDC4 (SEQ ID NO: 470). Other SDC4 polynucleotides comprise or consist of exons 1-5, 2-5, 3-5, 4-5, 1-4, 2-4, 3-4, 1-3, 2-3.

In another embodiment, the SDC4 polynucleotide comprises or consists of a fragment of at least 20 contiguous nucleotides of SEQ ID NO: 378, 380, 382, 383 or 470. Preferably the fragment comprises or consists of at least about 30, at least about 40, at least about 50, at least about 60, at least about 70, at least about 80, at least about 90, at least about 100 or more contiguous nucleotides of SEQ ID NO: 378, 380, 382, 383 or 470.

The SDC4 polynucleotide may comprise or consist of a variant of one of these sequences. In an embodiment, the SDC4 polynucleotide may comprise one or more (i.e. 1, 2, 3, 4, 5, 6, 7, 8, 9, 10 or more) point mutations that add, delete or substitute any of the nucleotides of SEQ ID NOs: 377, 378, 379, 380, 381, 382, 383 or 470. In another embodiment, the SDC4 polynucleotide has at least about 70%, at least about 80%, at least about 85%, at least about 90%, at least about 95%, at least about 97%, at least about 98% or at least about 99% sequence identity to any of SEQ ID NOs: 377, 378, 379, 380, 381, 382, 383 or 470. Sequence identity is typically measured over the full length of the non-variant sequence. Methods for measuring sequence identity are discussed in more detail below. In another embodiment, the SDC4 polynucleotide comprises or consists of a fragment of at least 20 contiguous nucleotides of a variant sequence having at least about 70% sequence identity or any of the other % sequence identities listed above to any of SEQ ID NOs: 377, 378, 379, 380, 381, 382, 383 or 470. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 377, 378, 379, 380, 381, 382, 383 or 470.

### SLC4A4

SLC4A4 or Solute Carrier Family 4 Member 4 is also known as NBC1; HNBC1; HhNMC; NBC2; PNBC; Solute Carrier Family 4 (Sodium Bicarbonate Cotransporter), Member 4; Electrogenic Sodium Bicarbonate Cotransporter 1; Na(+)/HCO3(−) Cotransporter; SLC4A5; KNBC1; Sodium Bicarbonate Cotransporter 1 (Sodium Bicarbonate Cotransporter, Kidney; Sodium Bicarbonate Cotransporter, Pancreas); Solute Carrier Family 4, Sodium Bicarbonate Cotransporter, Member 4, Brain Type; Solute Carrier Family 4, Sodium Bicarbonate Cotransporter, Member 4; Solute Carrier Family 4, Sodium Bicarbonate Cotransporter, Member 5; Sodium Bicarbonate Cotransporter; NBCe1-A; NBCE1; KNBC; and NBC. External Ids for SLC4A4 Gene are HGNC: 11030; NCBI Entrez Gene: 8671; Ensembl: ENSG00000080493; OMIM®: 603345; and UniProtKB/Swiss-Prot: Q9Y6R1.

In an embodiment, the SLC4A4 polynucleotide comprises the sequence of NM_001098484.3 (SEQ ID NO: 411), which comprises exons 1-26 of SLC4A4. In an embodiment, the SLC4A4 polynucleotide consists of the sequence of NM_001098484.3 (SEQ ID NO: 411), which comprises exons 1-26 of SLC4A4. In disclosed embodiments, said SLC4A4 polynucleotide is used for detecting a SLC4A4-NRG1 fusion polynucleotide as further discussed herein below. In disclosed embodiments, said detecting comprises (i) the sequence of SEQ ID NO: 384, (ii) a sequence having at least about 70% sequence identity to SEQ ID NO: 384, or (iii) a fragment comprising at least about 20 contiguous nucleotides of (i) or (ii), including at least the nucleotides at positions 75 and 76 of SEQ ID NO: 384.

In another embodiment, the SLC4A4 polynucleotide comprises or consists of any one or more of exons 1-26 of SLC4A4, recited herein as SEQ ID NOs: 385, 386, 387, 388, 389, 390, 391, 392, 393, 394, 395, 396, 397, 398, 399, 400, 401, 402, 403, 404, 405, 406, 407, 408, 409 and 410, respectively. In a disclosed embodiment, the SLC4A4 polynucleotide comprises or consists of exon 14 of SLC4A4 (SEQ ID NO: 398).

In a disclosed embodiment, the SLC4A4 polynucleotide comprises or consists of exons 1-14 of SLC4A4 (SEQ ID NO: 412). Other SLC4A4 polynucleotides comprise or consist of exons 2-14, 3-14, 4-14, 5-14, 6-14, 7-14, 8-14, 9-14, 10-14, 11-14, 12-14, 13-14, 1-13, 2-13, 3-13, 4-13, 5-13, 6-13, 7-13, 8-13, 9-13, 10-13, 11-13, 12-13, 1-12, 2-12, 3-12, 4-12, 5-12, 6-12, 7-12, 8-12, 9-12, 10-12, 11-12, 1-11, 2-11, 3-11, 4-11, 5-11, 6-11, 7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2.

In another embodiment, the SLC4A4 polynucleotide comprises or consists of a fragment of at least 20 contiguous nucleotides of SEQ ID NO: 398, 411 or 412. Preferably the fragment comprises or consists of at least about 30, at least about 40, at least about 50, at least about 60, at least about 70, at least about 80, at least about 90, at least about 100 or more contiguous nucleotides of SEQ ID NO: 398, 411 or 412.

The SLC4A4 polynucleotide may comprise or consist of a variant of one of these sequences. In an embodiment, the SLC4A4 polynucleotide may comprise one or more (i.e. 1, 2, 3, 4, 5, 6, 7, 8, 9, 10 or more) point mutations that add, delete or substitute any of the nucleotides of SEQ ID NOs: 385, 386, 387, 388, 389, 390, 391, 392, 393, 394, 395, 396, 397, 398, 399, 400, 401, 402, 403, 404, 405, 406, 407, 408, 409, 410, 411 or 412. In another embodiment, the SLC4A4 polynucleotide has at least about 70%, at least about 80%, at least about 85%, at least about 90%, at least about 95%, at least about 97%, at least about 98% or at least about 99% sequence identity to any of SEQ ID NOs: 385, 386, 387, 388, 389, 390, 391, 392, 393, 394, 395, 396, 397, 398, 399, 400, 401, 402, 403, 404, 405, 406, 407, 408, 409, 410, 411 or 412. Sequence identity is typically measured over the full length of the non-variant sequence. Methods for measuring sequence identity are discussed in more detail below. In another embodiment, the SLC4A4 polynucleotide comprises or consists of a fragment of at least 20 contiguous nucleotides of a variant sequence having at least about 70% sequence identity or any of the other % sequence identities listed above to any of SEQ ID NOs: 385, 386, 387, 388, 389, 390, 391, 392, 393, 394, 395, 396, 397, 398, 399, 400, 401, 402, 403, 404, 405, 406, 407, 408, 409, 410, 411 or 412. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 385, 386, 387, 388, 389, 390, 391, 392, 393, 394, 395, 396, 397, 398, 399, 400, 401, 402, 403, 404, 405, 406, 407, 408, 409, 410, 411 or 412.

### ZFAT

ZFAT or Zing Finger And AT-Hook Domain Containing is also known as Zinc Finger Protein 406; KIAA1485; ZNF406; ZFAT1; Zinc Finger Protein ZFAT; Zinc Finger Gene In Autoimmune Thyroid Disease; Zinc Finger Gene In AITD Susceptibility Region; and AITD3. External Ids for ZFAT Gene are HGNC: 19899; NCBI Entrez Gene: 57623; Ensembl: ENSG00000066827; OMIM®: 610931; and UniProtKB/Swiss-Prot: Q9P243.

In an embodiment, the ZFAT polynucleotide comprises the sequence of NM_020863.4 (SEQ ID NO: 430), which comprises exons 1-16 of ZFAT. In an embodiment, the ZFAT polynucleotide consists of the sequence of NM_020863.4 (SEQ ID NO: 430), which comprises exons 1-16 of ZFAT. In disclosed embodiments, the ZFAT polynucleotide is used for detecting a ZFAT-NRG1 fusion polynucleotide as further discussed herein below. In disclosed embodiments, said detecting comprises (i) the sequence of SEQ ID NO: 413, (ii) a sequence having at least about 70% sequence identity to SEQ ID NO: 413, or (iii) a fragment comprising at least about 20 contiguous nucleotides of (i) or (ii), including at least the nucleotides at positions 75 and 76 of SEQ ID NO: 413.

In another embodiment, the ZFAT polynucleotide comprises or consists of any one or more of exons 1-16 of ZFAT, recited herein as SEQ ID NOs: 414, 415, 416, 417, 418, 419, 420, 421, 422, 423, 424, 425, 426, 427, 428 and 429, respectively. In a disclosed embodiment, the ZFAT polynucleotide comprises or consists of exon 12 of ZFAT (SEQ ID NO: 425).

In a disclosed embodiment, the ZFAT polynucleotide comprises or consists of exons 1-12 of ZFAT (SEQ ID NO: 431). Other ZFAT polynucleotides comprise or consist of exons 1-16, 2-16, 3-16, 4-16, 5-16, 6-16, 7-16, 8-16, 9-16, 10-16, 11-16, 12-16, 13-16, 14-16, 15-16, 1-15, 2-15, 3-15, 4-15, 5-15, 6-15, 7-15, 8-15, 9-15, 10-15, 11-15, 12-15, 13-15, 14-15, 1-14, 2-14, 3-14, 4-14, 5-14, 6-14, 7-14, 8-14, 9-14, 10-14, 11-14, 12-14, 13-14, 1-13, 2-13, 3-13, 4-13, 5-13, 6-13, 7-13, 8-13, 9-13, 10-13, 11-13, 12-13, 2-12, 3-12, 4-12, 5-12, 6-12, 7-12, 8-12, 9-12, 10-12, 11-12, 1-11, 2-11, 3-11, 4-11, 5-11, 6-11, 7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2.

In another embodiment, the ZFAT polynucleotide comprises or consists of a fragment of at least 20 contiguous nucleotides of SEQ ID NO: 425, 430 or 431. Preferably the fragment comprises or consists of at least about 30, at least about 40, at least about 50, at least about 60, at least about 70, at least about 80, at least about 90, at least about 100 or more contiguous nucleotides of SEQ ID NO: 425, 430 or 431.

The ZFAT polynucleotide may comprise or consist of a variant of one of these sequences. In an embodiment, the ZFAT polynucleotide may comprise one or more (i.e. 1, 2, 3, 4, 5, 6, 7, 8, 9, 10 or more) point mutations that add, delete or substitute any of the nucleotides of SEQ ID NOs: 414, 415, 416, 417, 418, 419, 420, 421, 422, 423, 424, 425, 426, 427, 428, 429, 430 or 431. In another embodiment, the ZFAT polynucleotide has at least about 70%, at least about 80%, at least about 85%, at least about 90%, at least about 95%, at least about 97%, at least about 98% or at least about 99% sequence identity to any of SEQ ID NOs: 414, 415, 416, 417, 418, 419, 420, 421, 422, 423, 424, 425, 426, 427, 428, 429, 430 or 431. Sequence identity is typically measured over the full length of the non-variant sequence. Methods for measuring sequence identity are discussed in more detail below. In another embodiment, the ZFAT polynucleotide comprises or consists of a fragment of at least 20 contiguous nucleotides of a variant sequence having at least about 70% sequence identity or any of the other % sequence identities listed above to any of SEQ ID NOs: 414, 415, 416, 417, 418, 419, 420, 421, 422, 423, 424, 425, 426, 427, 428, 429, 430 or 431. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 414, 415, 416, 417, 418, 419, 420, 421, 422, 423, 424, 425, 426, 427, 428, 429, 430 or 431.

### DSCAML1

DSCAML1 or DS Cell Adhesion Molecule Like 1 is also known as KIAA1132; Down Syndrome Cell Adhesion Molecule-Like Protein 1; Down Syndrome Cell Adhesion Molecule 2; DSCAM2; Downs Syndrome Cell Adhesion Molecule Like 1; Down Syndrome Cell Adhesion Molecule Like 1; and DSCAM-Like 1. External Ids for DSCAML1 Gene are HGNC: 14656; NCBI Entrez Gene: 57453; Ensembl: ENSG00000177103; OMIM®: 611782; and UniProtKB/Swiss-Prot: Q8TD84.

In an embodiment, the DSCAML1 polynucleotide comprises the sequence of NM_020693.4 (SEQ ID NO: 466), which comprises exons 1-33 of DSCAML1. In an embodiment, the DSCAML1 polynucleotide consists of the sequence of NM_020693.4 (SEQ ID NO: 466), which comprises exons 1-33 of DSCAML1. In disclosed embodiments, the DSCAML1 polynucleotide is used for detecting a DSCAML1-NRG1 fusion polynucleotide as further discussed herein below. In disclosed embodiments, said detecting comprises (i) the sequence of SEQ ID NO: 432, (ii) a sequence having at least about 70% sequence identity to SEQ ID NO: 432, or (iii) a fragment comprising at least about 20 contiguous nucleotides of (i) or (ii), including at least the nucleotides at positions 75 and 76 of SEQ ID NO: 432.

In another embodiment, the DSCAML1 polynucleotide comprises or consists of any one or more of exons 1-33 of DSCAML1, recited herein as SEQ ID NOs: 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464 and 465, respectively. In a disclosed embodiment, the DSCAML1 polynucleotide comprises or consists of exon 3 of DSCAML1 (SEQ ID NO: 435). Other disclosed DSCAML1 polynucleotides comprise or consist of exons 1-3, 2-3, or 1-2.

In a disclosed embodiment, the DSCAML1 polynucleotide comprises or consists of exons 1-3 of DSCAML1 (SEQ ID NO: 467). Other DSCAML1 polynucleotides comprise or consist of exons 2-33, 3-33, 4-33, 5-33, 6-33, 7-33, 8-33, 9-33, 10-33, 11-33, 12-33, 13-33, 14-33, 15-33, 16-33, 17-33, 18-33, 19-33, 20-33, 21-33, 22-33, 23-33, 24-33, 25-33, 26-33, 27-33, 28-33, 29-33, 30-33, 31-33, 32-33, 1-32, 2-32, 3-32, 4-32, 5-32, 6-32, 7-32, 8-32, 9-32, 10-32, 11-32, 12-32, 13-32, 14-32, 15-32, 16-32, 17-32, 18-32, 19-32, 20-32, 21-32, 22-32, 23-32, 24-32, 25-32, 26-32, 27-32, 28-32, 29-32, 30-32, 31-32, 1-31, 2-31, 3-31, 4-31, 5-31, 6-31, 7-31, 8-31, 9-31, 10-31, 11-31, 12-31, 13-31, 14-31, 15-31, 16-31, 17-31, 18-31, 19-31, 20-31, 21-31, 22-31, 23-31, 24-31, 25-, 31 26-31, 27-31, 28-31, 29-31, 30-31, 1-30, 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, 29-30, 1-29, 2-29, 3-29, 4-29, 5-29, 6-29, 7-29, 8-29, 9-29, 10-29, 11-29, 12-29, 13-29, 14-29, 15-29, 16-29, 17-29, 18-29, 19-29, 20-29, 21-29, 22-29, 23-29, 24-29, 25-29, 26-29, 27-29, 28-29, 1-28, 2-28, 3-28, 4-28, 5-28, 6-28, 7-28, 8-28, 9-28, 10-28, 11-28, 12-28, 13-28, 14-28, 15-28, 16-28, 17-28, 18-28, 19-28, 20-28, 21-28, 22-28, 23-28, 24-28, 25-28, 26-28, 27-28, 1-27, 2-27, 3-27, 4-27, 5-27, 6-27, 7-27, 8-27, 9-27, 10-27, 11-27, 12-27, 13-27, 14-27, 15-27, 16-27, 17-27, 18-27, 19-27, 20-27, 21-27, 22-27, 23-27, 24-27, 25-27, 26-27, 1-26, 2-26, 3-26, 4-26, 5-26, 6-26, 7-26, 8-26, 9-26, 10-26, 11-26, 12-26, 13-26, 14-26, 15-26, 16-26, 17-26, 18-26, 19-26, 20-26, 21-26, 22-26, 23-26, 24-26, 25-26, 1-25, 2-25, 3-25, 4-25, 5-25, 6-25, 7-25, 8-25, 9-25, 10-25, 11-25, 12-25, 13-25, 14-25, 15-25, 16-25, 17-25, 18-25, 19-25, 20-25, 21-25, 22-25, 23-25, 24-25, 1-24, 2-24, 3-24, 4-24, 5-24, 6-24, 7-24, 8-24, 9-24, 10-24, 11-24, 12-24, 13-24, 14-24, 15-24, 16-24, 17-24, 18-24, 19-24, 20-24, 21-24, 22-24, 23-24, 1-23, 2-23, 3-23, 4-23, 5-23, 6-23, 7-23, 8-23, 9-23, 10-23, 11-23, 12-23, 13-23, 14-23, 15-23, 16-23, 17-23, 18-23, 19-23, 20-23, 21-23, 22-23, 1-22, 2-22, 3-22, 4-22, 5-22, 6-22, 7-22, 8-22, 9-22, 10-22, 11-22, 12-22, 13-22, 14-22, 15-22, 16-22, 17-22, 18-22, 19-22, 20-22, 21-22, 1-21, 2-21, 3-21, 4-21, 5-21, 6-21, 7-21, 8-21, 9-21, 10-21, 11-21, 12-21, 13-21, 14-21, 15-21, 16-21, 17-21, 18-21, 19-21, 20-21, 1-20, 2-20, 3-20, 4-20, 5-20, 6-20, 7-20, 8-20, 9-20, 10-20, 11-20, 12-20, 13-20, 14-20, 15-20, 16-20, 17-20, 18-20, 19-20, 1-19, 2-19, 3-19, 4-19, 5-19, 6-19, 7-19, 8-19, 9-19, 10-19, 11-19, 12-19, 13-19, 14-19, 15-19, 16-19, 17-19, 18-19, 1-18, 2-18, 3-18, 4-18, 5-18, 6-18, 7-18, 8-18, 9-18, 10-18, 11-18, 12-18, 13-18, 14-18, 15-18, 16-18, 17-18, 1-17, 2-17, 3-17, 4-17, 5-17, 6-17, 7-17, 8-17, 9-17, 10-17, 11-17, 12-17, 13-17, 14-17, 15-17, 16-17, 1-16, 2-16, 3-16, 4-16, 5-16, 6-16, 7-16, 8-16, 9-16, 10-16, 11-16, 12-16, 13-16, 14-16, 15-16, 1-15, 2-15, 3-15, 4-15, 5-15, 6-15, 7-15, 8-15, 9-15, 10-15, 11-15, 12-15, 13-15, 14-15, 1-14, 2-14, 3-14, 4-14, 5-14, 6-14, 7-14, 8-14, 9-14, 10-14, 11-14, 12-14, 13-14, 1-13, 2-13, 3-13, 4-13, 5-13, 6-13, 7-13, 8-13, 9-13, 10-13, 11-13, 12-13, 1-12, 2-12, 3-12, 4-12, 5-12, 6-12, 7-12, 8-12, 9-12, 10-12, 11-12, 1-11, 2-11, 3-11, 4-11, 5-11, 6-11, 7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4 or 3-4.

In another embodiment, the DSCAML1 polynucleotide comprises or consists of a fragment of at least 20 contiguous nucleotides of SEQ ID NO: 435, 466 or 467. Preferably the fragment comprises or consists of at least about 30, at least about 40, at least about 50, at least about 60, at least about 70, at least about 80, at least about 90, at least about 100 or more contiguous nucleotides of SEQ ID NO: 435, 466 or 467.

The DSCAML1 polynucleotide may comprise or consist of a variant of one of these sequences. In an embodiment, the DSCAML1 polynucleotide may comprise one or more (i.e. 1, 2, 3, 4, 5, 6, 7, 8, 9, 10 or more) point mutations that add, delete or substitute any of the nucleotides of SEQ ID NOs: 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466 or 467. In another embodiment, the DSCAML1 polynucleotide has at least about 70%, at least about 80%, at least about 85%, at least about 90%, at least about 95%, at least about 97%, at least about 98% or at least about 99% sequence identity to any of SEQ ID NOs: 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466 or 467. Sequence identity is typically measured over the full length of the non-variant sequence. Methods for measuring sequence identity are discussed in more detail below. In another embodiment, the DSCAML1 polynucleotide comprises or consists of a fragment of at least 20 contiguous nucleotides of a variant sequence having at least about 70% sequence identity or any of the other % sequence identities listed above to any of SEQ ID NOs: 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466 or 467. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466 or 467.

### RBPMS

RNA Binding Protein, MRNA Processing Factor (or RBPMS) is also known as HERMES, RNA Binding Protein With Multiple Splicing, RNA-Binding Protein With Multiple Splicing, Heart And RRM Expressed Sequence or RBP-MS. External Ids for RBPMS are HGNC: 19097 NCBI Entrez Gene: 11030 Ensembl: ENSG00000157110 OMIM: 601558 UniProtKB/Swiss-Prot: Q93062.

In an embodiment, the RBPMS polynucleotide comprises the sequence of NM_006867.4 (SEQ ID NO: 481), which comprises exons 1-10 of RBPMS. In an embodiment, the RBPMS polynucleotide consists of the sequence of NM_006867.4 (SEQ ID NO: 481), which comprises exons 1-10 of RBPMS.

In another embodiment, the RBPMS polynucleotide comprises or consists of any one or more of exons 1-10 of RBPMS, recited herein as SEQ ID NOs: 471, 472, 473, 474, 475, 476, 477, 478, 479 and 480, respectively. In a disclosed embodiment, the RPBMS polynucleotide comprises or consists of exon 5 of RBPMS (SEQ ID NO: 475).

In a disclosed embodiment, the RBPMS polynucleotide comprises or consists of exons 1-5 of RBPMS (SEQ ID NO: 482). Other RBPMS polynucleotides comprise or consist of exons 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 2-5, 3-5, 4-5, 1-4, 2-4, 3-4, 1-3, 2-3, or 1-2.

In another embodiment, the RBPMS polynucleotide comprises or consists of a fragment of at least 20 contiguous nucleotides of SEQ ID NO: 475, 481 or 482. Preferably the fragment comprises or consists of at least about 30, at least about 40, at least about 50, at least about 60, at least about 70, at least about 80, at least about 90, at least about 100 or more contiguous nucleotides of SEQ ID NO: 475, 481 or 482.

The RBPMS polynucleotide may comprise or consist of a variant of one of these sequences. In an embodiment, the RBPMS polynucleotide may comprise one or more (i.e. 1, 2, 3, 4, 5, 6, 7, 8, 9, 10 or more) point mutations that add, delete or substitute any of the nucleotides of SEQ ID NOs: 471, 472, 473, 474, 475, 476, 477, 478, 479, 480, 481 or 482. In another embodiment, the RBPMS polynucleotide has at least about 70%, at least about 80%, at least about 85%, at least about 90%, at least about 95%, at least about 97%, at least about 98% or at least about 99% sequence identity to any of SEQ ID NOs: 471, 472, 473, 474, 475, 476, 477, 478, 479, 480, 481 or 482. Sequence identity is typically measured over the full length of the non-variant sequence. Methods for measuring sequence identity are discussed in more detail below. In another embodiment, the RBPMS polynucleotide comprises or consists of a fragment of at least 20 contiguous nucleotides of a variant sequence having at least about 70% sequence identity or any of the other % sequence identities listed above to any of SEQ ID NOs: 471, 472, 473, 474, 475, 476, 477, 478, 479, 480, 481 or 482. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 471, 472, 473, 474, 475, 476, 477, 478, 479, 480, 481 or 482.

### ATP1B1

ATP1B1 or ATPase Na+/K+ Transporting Subunit Beta is also known as Sodium/Potassium-Transporting ATPase Subunit Beta, ATP1B, Sodium-Potassium ATPase Subunit Beta 1 (Non-Catalytic), ATPase, Na+/K+ Transporting, Beta 1 Polypeptide, Sodium Pump Subunit Beta. External Ids for ATP1B1 include HGNC: 804 NCBI Entrez Gene: 481 Ensembl: ENSG00000143153 OMIM: 182330 UniProtKB/Swiss-Prot: P05026.

In an embodiment, the ATP1B1 polynucleotide comprises the sequence of NM_001677.4 (SEQ ID NO: 489), which comprises exons 1-6 of ATP1B1. In an embodiment, the ATP1B1 polynucleotide consists of the sequence of NM_001677.4 (SEQ ID NO: 489), which comprises exons 1-6 of ATP1B1.

In another embodiment, the ATP1B1 polynucleotide comprises or consists of any one or more of exons 1-6 of ATP1B1, recited herein as SEQ ID NOs: 483, 484, 485, 486, 487 and 488, respectively. In a disclosed embodiment, the ATP1B1 polynucleotide comprises or consists of exon 2 of ATP1B1 (SEQ ID NO: 484).

In a disclosed embodiment, the ATP1B1 polynucleotide comprises or consists of exons 1-2 of ATP1B1 (SEQ ID NO: 490). Other ATP1B1 polynucleotides comprise or consist of exons 1-6, 2-6, 3-6, 4-6, 5-6, 2-5, 3-5, 4-5, 1-4, 2-4, 3-4, 1-3 or 2-3.

In another embodiment, the ATP1B1 polynucleotide comprises or consists of a fragment of at least 20 contiguous nucleotides of SEQ ID NO: 484, 489 or 490. Preferably the fragment comprises or consists of at least about 30, at least about 40, at least about 50, at least about 60, at least about 70, at least about 80, at least about 90, at least about 100 or more contiguous nucleotides of SEQ ID NO: 484, 489 or 490.

The ATP1B1 polynucleotide may comprise or consist of a variant of one of these sequences. In an embodiment, the ATP1B1 polynucleotide may comprise one or more (i.e. 1, 2, 3, 4, 5, 6, 7, 8, 9, 10 or more) point mutations that add, delete or substitute any of the nucleotides of SEQ ID NOs: 483, 484, 485, 486, 487, 488, 489 or 490. In another embodiment, the ATP1B1 polynucleotide has at least about 70%, at least about 80%, at least about 85%, at least about 90%, at least about 95%, at least about 97%, at least about 98% or at least about 99% sequence identity to any of SEQ ID NOs: 483, 484, 485, 486, 487, 488, 489 or 490. Sequence identity is typically measured over the full length of the non-variant sequence. Methods for measuring sequence identity are discussed in more detail below. In another embodiment, the ATP1B1 polynucleotide comprises or consists of a fragment of at least 20 contiguous nucleotides of a variant sequence having at least about 70% sequence identity or any of the other % sequence identities listed above to any of SEQ ID NOs: 483, 484, 485, 486, 487, 488, 489 or 490. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 483, 484, 485, 486, 487, 488, 489 or 490.

### Specific NRG1 Fusion Polynucleotides

As noted above, NRG1 fusion polynucleotides disclosed and detected by the products and methods of embodiments of the invention disclosed herein include VAPB-NRG1 (i.e. comprising or consisting of a VAPB polynucleotide and an NRG1 polynucleotide), CADM1-NRG1 (i.e. comprising or consisting of a CADM1 polynucleotide and an NRG1 polynucleotide), CD44-NRG1 (i.e. comprising or consisting of a CD44 polynucleotide and an NRG1 polynucleotide), SLC3A2-NRG1 (i.e. comprising or consisting of a SLC3A2 polynucleotide and an NRG1 polynucleotide), VTCN1-NRG1 (i.e. comprising or consisting of a VTCN1 polynucleotide and an NRG1 polynucleotide), CDH1-NRG1 (i.e. comprising or consisting of a CDH1 polynucleotide and an NRG1 polynucleotide), CXADR-NRG1 (i.e. comprising or consisting of a CXADR polynucleotide and an NRG1 polynucleotide, GTF2E2-NRG1 (i.e. comprising or consisting of a GTF2E2 polynucleotide and an NRG1 polynucleotide, CSMD1-NRG1 (i.e. comprising or consisting of a CSMD1 polynucleotide and an NRG1 polynucleotide, PTN-NRG1 (i.e. comprising or consisting of a PTN polynucleotide and an NRG1 polynucleotide, ST14-NRG1 (i.e. comprising or consisting of an ST14 polynucleotide and an NRG1 polynucleotide, THBS1-NRG1 (i.e. comprising or consisting of a THBS1 polynucleotide and an NRG1 polynucleotide, AGRN-NRG1 (i.e. comprising or consisting of an AGRN polynucleotide and an NRG1 polynucleotide, PVALP-NRG1 (i.e. comprising or consisting of a PVALP polynucleotide and an NRG1 polynucleotide, APP-NRG1 (i.e. comprising or consisting of a APP polynucleotide and an NRG1 polynucleotide), WRN-NRG1 (i.e. comprising or consisting of a WRN polynucleotide and an NRG1 polynucleotide), DAAM1-NRG1 (i.e. comprising or consisting of a DAAM1polynucleotide and an NRG1 polynucleotide), ASPH-NRG1 (i.e. comprising or consisting of a ASPH polynucleotide and an NRG1 polynucleotide), NOTCH2-NRG1 (i.e. comprising or consisting of a NOTCH2 polynucleotide and an NRG1 polynucleotide), CD74-NRG1 (i.e. comprising or consisting of a CD74 polynucleotide and an NRG1 polynucleotide), SDC4-NRG1 (i.e. comprising or consisting of a SDC4 polynucleotide and an NRG1 polynucleotide), SLC4A4-NRG1 (i.e. comprising or consisting of a SLC4A4 polynucleotide and an NRG1 polynucleotide), ZFAT-NRG1 (i.e. comprising or consisting of a ZFAT polynucleotide and an NRG1 polynucleotide), DSCAML1-NRG1 (i.e. comprising or consisting of a DSCAML1 polynucleotide and an NRG1 polynucleotide), RBPMS-NRG1 (i.e. comprising or consisting of a RBPMS1 polynucleotide and an NRG1 polynucleotide) or ATP1B1-NRG1 (i.e. comprising or consisting of a ATP1B1 polynucleotide and an NRG1 polynucleotide).

In a disclosed embodiment, the NRG1 fusion polynucleotide is a VAPB-NRG1 fusion polynucleotide. The VAPB-NRG1 fusion polynucleotide preferably comprises or consists of the sequence of SEQ ID NO: 19 In another embodiment, the VAPB-NRG1 fusion polynucleotide comprises or consists of a fragment of at least 20 contiguous nucleotides of SEQ ID NO: 19, including at least the nucleotides at positions 43 and 44 of SEQ ID NO: 19. These specific nucleotides define the fusion junction. Preferably the fragment comprises or consists of at least about 30, at least about 40, at least about 50, at least about 60, at least about 70, at least about 80, at least about 90, at least about 100 or more contiguous nucleotides of SEQ ID NO: 19.

The VAPB-NRG1 fusion polynucleotide may be a variant of a of these sequences. In an embodiment, the VAPB-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 19. In another embodiment, the VAPB-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 19. Sequence identity is typically measured over the full length of the non-variant sequence. Methods for measuring sequence identity are discussed in more detail below. In another embodiment, the VAPB-NRG1 fusion polynucleotide comprises or consists of a fragment of at least 20 contiguous nucleotides of a variant sequence having at least about 70% sequence identity or any of the other % sequence identities listed above to SEQ ID NO: 19, including at least the nucleotides at positions 43 and 44 of SEQ ID NO: 19. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 19.

In another disclosed embodiment, the NRG1 fusion polynucleotide is a CADM1-NRG1 fusion polynucleotide. The CADM1-NRG1 fusion polynucleotide preferably comprises or consists of the sequence of SEQ ID NO: 27. In another embodiment, the CADM1-NRG1 fusion polynucleotide comprises or consists of a fragment of at least 20 contiguous nucleotides of SEQ ID NO: 27, including at least the nucleotides at positions 53 and 54 of SEQ ID NO: 27. These specific nucleotides define the fusion junction. Preferably the fragment comprises or consists of at least about 30, at least about 40, at least about 50, at least about 60, at least about 70, at least about 80, at least about 90, at least about 100 or more contiguous nucleotides of SEQ ID NO: 27.

The CADM1-NRG1 fusion polynucleotide may be a variant of one of these sequences. In an embodiment, the CADM1-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 27. In another embodiment, the CADM1-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 27.

Sequence identity is typically measured over the full length of the non-variant sequence. Methods for measuring sequence identity are discussed in more detail below. In another embodiment, the CADM1-NRG1 fusion polynucleotide comprises or consists of a fragment of at least 20 contiguous nucleotides of a variant sequence having at least about 70% sequence identity or any of the other % sequence identities listed above to SEQ ID NO: 27, including at least the nucleotides at positions 53 and 54 of SEQ ID NO: 27. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 27.

In another disclosed embodiment, the NRG1 fusion polynucleotide is a CD44-NRG1 fusion polynucleotide. The CD44-NRG1 fusion polynucleotide preferably comprises or consists of the sequence of SEQ ID NO: 41. In another embodiment, the CD44-NRG1 fusion polynucleotide comprises or consists of a fragment of at least 20 contiguous nucleotides of SEQ ID NO: 41, including at least the nucleotides at positions 52 and 53 of SEQ ID NO: 41. These specific nucleotides define the fusion junction. Preferably the fragment comprises or consists of at least about 30, at least about 40, at least about 50, at least about 60, at least about 70, at least about 80, at least about 90, at least about 100 or more contiguous nucleotides of SEQ ID NO: 41.

The CD44-NRG1 fusion polynucleotide may be a variant of one of these sequences. In an embodiment, the CD44-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 41. In another embodiment, the CD44-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 41. Sequence identity is typically measured over the full length of the non-variant sequence. Methods for measuring sequence identity are discussed in more detail below. In another embodiment, the CD44-NRG1 fusion polynucleotide comprises or consists of a fragment of at least 20 contiguous nucleotides of a variant sequence having at least about 70% sequence identity or any of the other % sequence identities listed above to SEQ ID NO: 41, including at least the nucleotides at positions 52 and 53 of SEQ ID NO: 41. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 41.

In another disclosed embodiment, the CD44-NRG1 fusion polynucleotide comprises or consists of the sequence of SEQ ID NO: 469. In another embodiment, the CD44-NRG1 fusion polynucleotide comprises or consists of a fragment of at least 20 contiguous nucleotides of SEQ ID NO: 469, including at least the nucleotides at positions 75 and 76 of SEQ ID NO: 469. These specific nucleotides define the fusion junction. Preferably the fragment comprises or consists of at least about 30, at least about 40, at least about 50, at least about 60, at least about 70, at least about 80, at least about 90, at least about 100 or more contiguous nucleotides of SEQ ID NO: 469.

The CD44-NRG1 fusion polynucleotide may be a variant of one of these sequences. In an embodiment, the CD44-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 469. In another embodiment, the CD44-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 469. Sequence identity is typically measured over the full length of the non-variant sequence.

Methods for measuring sequence identity are discussed in more detail below. In another embodiment, the CD44-NRG1 fusion polynucleotide comprises or consists of a fragment of at least 20 contiguous nucleotides of a variant sequence having at least about 70% sequence identity or any of the other % sequence identities listed above to SEQ ID NO: 469, including at least the nucleotides at positions 75 and 76 of SEQ ID NO: 469. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 469.

In another disclosed embodiment, the NRG1 fusion polynucleotide is a SLC3A2-NRG1 fusion polynucleotide. The SLC3A2-NRG1 fusion polynucleotide preferably comprises or consists of the sequence of SEQ ID NO: 62 or 225. In another embodiment, the SLC3A2-NRG1 fusion polynucleotide comprises or consists of a fragment of at least 20 contiguous nucleotides of SEQ ID NO: 62 or 225, including at least the nucleotides at positions 53 and 54 of SEQ ID NO: 62 or including at least the nucleotides at positions 93 and 94 of SEQ ID NO: 225.

These specific nucleotides define the fusion junction. Preferably the fragment comprises or consists of at least about 30, at least about 40, at least about 50, at least about 60, at least about 70, at least about 80, at least about 90, at least about 100 or more contiguous nucleotides of SEQ ID NO: 62 or 225.

The SLC3A2-NRG1 fusion polynucleotide may be a variant of one of these sequences. In an embodiment, the SLC3A2-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 62 or 225. In another embodiment, the SLC3A2-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 62 or 225. Sequence identity is typically measured over the full length of the non-variant sequence.

Methods for measuring sequence identity are discussed in more detail below. In another embodiment, the SLC3A2-NRG1 fusion polynucleotide comprises or consists of a fragment of at least 20 contiguous nucleotides of a variant sequence having at least about 70% sequence identity or any of the other % sequence identities listed above to SEQ ID NO: 62 or 225, including at least the nucleotides at positions 53 and 54 of SEQ ID NO: 62 or including at least the nucleotides at positions 93 and 94 of SEQ ID NO: 225. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 62 or 225.

In another disclosed embodiment, the NRG1 fusion polynucleotide comprises or consists of a VTCN1-NRG1 fusion polynucleotide. The VTCN1-NRG1 fusion polynucleotide preferably comprises or consists of the sequence of SEQ ID NO: 74. In another embodiment, the VTCN1-NRG1 fusion polynucleotide comprises or consists of a fragment of at least 20 contiguous nucleotides of SEQ ID NO: 74, including at least the nucleotides at positions 65 and 66 of SEQ ID NO: 74. These specific nucleotides define the fusion junction. Preferably the fragment comprises or consists of at least about 30, at least about 40, at least about 50, at least about 60, at least about 70, at least about 80, at least about 90, at least about 100 or more contiguous nucleotides of SEQ ID NO: 74.

The VTCN1-NRG1 fusion polynucleotide may be a variant of one of these sequences. In an embodiment, the VTCN1-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 74. In another embodiment, the VTCN1-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 74. Sequence identity is typically measured over the full length of the non-variant sequence. Methods for measuring sequence identity are discussed in more detail below. In another embodiment, the VTCN1-NRG1 fusion polynucleotide comprises or consists of a fragment of at least 20 contiguous nucleotides of a variant sequence having at least about 70% sequence identity or any of the other % sequence identities listed above to SEQ ID NO: 74, including at least the nucleotides at positions 65 and 66 of SEQ ID NO: 74. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 74.

In another disclosed embodiment, the NRG1 fusion polynucleotide comprises or consists of a CDH1-NRG1 fusion polynucleotide. The CDH1-NRG1 fusion polynucleotide preferably comprises or consists of the sequence of SEQ ID NO: 83. In another embodiment, the CDH1-NRG1 fusion polynucleotide comprises or consists of a fragment of at least 20 contiguous nucleotides of SEQ ID NO: 83, including at least the nucleotides at positions 119 and 120 of SEQ ID NO: 83. These specific nucleotides define the fusion junction. Preferably the fragment comprises or consists of at least about 30, at least about 40, at least about 50, at least about 60, at least about 70, at least about 80, at least about 90, at least about 100 or more contiguous nucleotides of SEQ ID NO: 83.

The CDH1-NRG1 fusion polynucleotide may be a variant of one of these sequences. In an embodiment, the CDH1-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 83. In another embodiment, the CDH1-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 83. Sequence identity is typically measured over the full length of the non-variant sequence. Methods for measuring sequence identity are discussed in more detail below. In another embodiment, the CDH1-NRG1 fusion polynucleotide comprises or consists of a fragment of at least 20 contiguous nucleotides of a variant sequence having at least about 70% sequence identity or any of the other % sequence identities listed above to SEQ ID NO: 83, including at least the nucleotides at positions 119 and 120 of SEQ ID NO: 83. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 83.

In another disclosed embodiment, the NRG1 fusion polynucleotide comprises or consists of a CXADR-NRG1 fusion polynucleotide. The CXADR-NRG1 fusion polynucleotide preferably comprises or consists of the sequence of SEQ ID NO: 102. In another embodiment, the CXADR-NRG1 fusion polynucleotide comprises or consists of a fragment of at least 20 contiguous nucleotides of SEQ ID NO: 102, including at least the nucleotides at positions 43 and 44 of SEQ ID NO: 102. These specific nucleotides define the fusion junction. Preferably the fragment comprises or consists of at least about 30, at least about 40, at least about 50, at least about 60, at least about 70, at least about 80, at least about 90, at least about 100 or all contiguous nucleotides of SEQ ID NO: 102.

The CXADR-NRG1 fusion polynucleotide may be a variant of one of these sequences. In an embodiment, the CXADR-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 102. In another embodiment, the CXADR-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 102. Sequence identity is typically measured over the full length of the non-variant sequence. Methods for measuring sequence identity are discussed in more detail below. In another embodiment, the CXADR-NRG1 fusion polynucleotide comprises or consists of a fragment of at least 20 contiguous nucleotides of a variant sequence having at least about 70% sequence identity or any of the other % sequence identities listed above to SEQ ID NO: 102, including at least the nucleotides at positions 43 and 44 of SEQ ID NO: 102. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 102.

In another disclosed embodiment, the NRG1 fusion polynucleotide comprises or consists of a GTF2E2-NRG1 fusion polynucleotide. The GTF2E2-NRG1 fusion polynucleotide preferably comprises or consists of the sequence of SEQ ID NO: 109. In another embodiment, the GTF2E2-NRG1 fusion polynucleotide comprises or consists of a fragment of at least 20 contiguous nucleotides of SEQ ID NO: 109, including at least the nucleotides at positions 141 and 142 of SEQ ID NO: 109. These specific nucleotides define the fusion junction. Preferably the fragment comprises or consists of at least about 30, at least about 40, at least about 50, at least about 60, at least about 70, at least about 80, at least about 90, at least about 100 or all contiguous nucleotides of SEQ ID NO: 109.

The GTF2E2-NRG1 fusion polynucleotide may be a variant of one of these sequences. In an embodiment, the GTF2E2-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 109. In another embodiment, the GTF2E2-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 109. Sequence identity is typically measured over the full length of the non-variant sequence. Methods for measuring sequence identity are discussed in more detail below. In another embodiment, the GTF2E2-NRG1 fusion polynucleotide comprises or consists of a fragment of at least 20 contiguous nucleotides of a variant sequence having at least about 70% sequence identity or any of the other % sequence identities listed above to SEQ ID NO: 109, including at least the nucleotides at positions 141 and 142 of SEQ ID NO: 109. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 109.

In another disclosed embodiment, the NRG1 fusion polynucleotide comprises or consists of a CSMD1-NRG1 fusion polynucleotide. The CSMD1-NRG1 fusion polynucleotide preferably comprises or consists of the sequence of SEQ ID NO: 120. In another embodiment, the CSMD1-NRG1 fusion polynucleotide comprises or consists of a fragment of at least 20 contiguous nucleotides of SEQ ID NO: 120, including at least the nucleotides at positions 88 and 89 of SEQ ID NO: 120. These specific nucleotides define the fusion junction. Preferably the fragment comprises or consists of at least about 30, at least about 40, at least about 50, at least about 60, at least about 70, at least about 80, at least about 90, at least about 100 or all contiguous nucleotides of SEQ ID NO: 120.

The CSMD1-NRG1 fusion polynucleotide may be a variant of one of these sequences. In an embodiment, the CSMD1-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 120. In another embodiment, the CSMD1-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 120. Sequence identity is typically measured over the full length of the non-variant sequence. Methods for measuring sequence identity are discussed in more detail below. In another embodiment, the CSMD1-NRG1 fusion polynucleotide comprises or consists of a fragment of at least 20 contiguous nucleotides of a variant sequence having at least about 70% sequence identity or any of the other % sequence identities listed above to SEQ ID NO: 120, including at least the nucleotides at positions 88 and 89 of SEQ ID NO: 120. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 120.

In another disclosed embodiment, the NRG1 fusion polynucleotide comprises or consists of a PTN-NRG1 fusion polynucleotide. The PTN-NRG1 fusion polynucleotide preferably comprises or consists of the sequence of SEQ ID NO: 146. In another embodiment, the PTN-NRG1 fusion polynucleotide comprises or consists of a fragment of at least 20 contiguous nucleotides of SEQ ID NO: 146, including at least the nucleotides at positions 102 and 103 of SEQ ID NO: 146. These specific nucleotides define the fusion junction. Preferably the fragment comprises or consists of at least about 30, at least about 40, at least about 50, at least about 60, at least about 70, at least about 80, at least about 90, at least about 100 or all contiguous nucleotides of SEQ ID NO: 146.

The PTN-NRG1 fusion polynucleotide may be a variant of one of these sequences. In an embodiment, the PTN-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 146. In another embodiment, the PTN-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 146. Sequence identity is typically measured over the full length of the non-variant sequence. Methods for measuring sequence identity are discussed in more detail below. In another embodiment, the PTN-NRG1 fusion polynucleotide comprises or consists of a fragment of at least 20 contiguous nucleotides of a variant sequence having at least about 70% sequence identity or any of the other % sequence identities listed above to SEQ ID NO: 146, including at least the nucleotides at positions 102 and 103 of SEQ ID NO: 146. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 146.

In another disclosed embodiment, the NRG1 fusion polynucleotide comprises or consists of a ST14-NRG1 fusion polynucleotide. The ST14-NRG1 fusion polynucleotide preferably comprises or consists of the sequence of SEQ ID NO: 154. In another embodiment, the ST14-NRG1 fusion polynucleotide comprises or consists of a fragment of at least 20 contiguous nucleotides of SEQ ID NO: 154, including at least the nucleotides at positions 95 and 96 of SEQ ID NO: 154. These specific nucleotides define the fusion junction. Preferably the fragment comprises or consists of at least about 30, at least about 40, at least about 50, at least about 60, at least about 70, at least about 80, at least about 90, at least about 100 or all contiguous nucleotides of SEQ ID NO: 154.

The ST14-NRG1 fusion polynucleotide may be a variant of one of these sequences. In an embodiment, the ST14-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 154. In another embodiment, the ST14-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 154. Sequence identity is typically measured over the full length of the non-variant sequence. Methods for measuring sequence identity are discussed in more detail below. In another embodiment, the ST14-NRG1 fusion polynucleotide comprises or consists of a fragment of at least 20 contiguous nucleotides of a variant sequence having at least about 70% sequence identity or any of the other % sequence identities listed above to SEQ ID NO: 154, including at least the nucleotides at positions 95 and 96 of SEQ ID NO: 154. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 154.

In another disclosed embodiment, the NRG1 fusion polynucleotide comprises or consists of a THBS1-NRG1 fusion polynucleotide. The THBS1-NRG1 fusion polynucleotide preferably comprises or consists of the sequence of SEQ ID NO: 176. In another embodiment, the THBS1-NRG1 fusion polynucleotide comprises or consists of a fragment of at least 20 contiguous nucleotides of SEQ ID NO: 176, including at least the nucleotides at positions 56 and 57 of SEQ ID NO: 176. These specific nucleotides define the fusion junction. Preferably the fragment comprises or consists of at least about 30, at least about 40, at least about 50, at least about 60, at least about 70, at least about 80, at least about 90, at least about 100 or all contiguous nucleotides of SEQ ID NO: 176.

The THBS1-NRG1 fusion polynucleotide may be a variant of one of these sequences. In an embodiment, the THBS1-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 176. In another embodiment, the THBS1-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 176. Sequence identity is typically measured over the full length of the non-variant sequence. Methods for measuring sequence identity are discussed in more detail below. In another embodiment, the THBS1-NRG1 fusion polynucleotide comprises or consists of a fragment of at least 20 contiguous nucleotides of a variant sequence having at least about 70% sequence identity or any of the other % sequence identities listed above to SEQ ID NO: 176, including at least the nucleotides at positions 56 and 57 of SEQ ID NO: 176. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 176.

In another disclosed embodiment, the NRG1 fusion polynucleotide comprises or consists of an AGRN-NRG1 fusion polynucleotide. The AGRN-NRG1 fusion polynucleotide preferably comprises or consists of the sequence of SEQ ID NO: 201. In another embodiment, the AGRN-NRG1 fusion polynucleotide comprises or consists of a fragment of at least 20 contiguous nucleotides of SEQ ID NO: 201, including at least the nucleotides at positions 106 and 107 of SEQ ID NO: 201. These specific nucleotides define the fusion junction.

Preferably the fragment comprises or consists of at least about 30, at least about 40, at least about 50, at least about 60, at least about 70, at least about 80, at least about 90, at least about 100 or all contiguous nucleotides of SEQ ID NO: 201.

The AGRN-NRG1 fusion polynucleotide may be a variant of one of these sequences. In an embodiment, the AGRN-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 201. In another embodiment, the AGRN-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 201. Sequence identity is typically measured over the full length of the non-variant sequence. Methods for measuring sequence identity are discussed in more detail below. In another embodiment, the AGRN-NRG1 fusion polynucleotide comprises or consists of a fragment of at least 20 contiguous nucleotides of a variant sequence having at least about 70% sequence identity or any of the other % sequence identities listed above to SEQ ID NO: 201, including at least the nucleotides at positions 106 and 107 of SEQ ID NO: 201. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 201.

In another disclosed embodiment, the NRG1 fusion polynucleotide comprises or consists of a PVALB-NRG1 fusion polynucleotide. The PVALB-NRG1 fusion polynucleotide preferably comprises or consists of the sequence of SEQ ID NO: 217. In another embodiment, the PVALB-NRG1 fusion polynucleotide comprises or consists of a fragment of at least 20 contiguous nucleotides of SEQ ID NO: 217, including at least the nucleotides at positions 102 and 103 of SEQ ID NO: 217. These specific nucleotides define the fusion junction. Preferably the fragment comprises or consists of at least about 30, at least about 40, at least about 50, at least about 60, at least about 70, at least about 80, at least about 90, at least about 100 or all contiguous nucleotides of SEQ ID NO: 217.

The PVALB-NRG1 fusion polynucleotide may be a variant of one of these sequences. In an embodiment, the PVALB-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 217. In another embodiment, the PVALB-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 217. Sequence identity is typically measured over the full length of the non-variant sequence. Methods for measuring sequence identity are discussed in more detail below. In another embodiment, the PVALB-NRG1 fusion polynucleotide comprises or consists of a fragment of at least 20 contiguous nucleotides of a variant sequence having at least about 70% sequence identity or any of the other % sequence identities listed above to SEQ ID NO: 217, including at least the nucleotides at positions 102 and 103 of SEQ ID NO: 217. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 217.

In disclosed embodiment, the NRG1 fusion polynucleotide comprises or consists of a APP-NRG1 fusion polynucleotide. The APP-NRG1 fusion polynucleotide preferably comprises or consists of the sequence of SEQ ID NO: 240. In another embodiment, the APP-NRG1 fusion polynucleotide comprises or consists of a fragment of at least 20 contiguous nucleotides of SEQ ID NO: 240, including at least the nucleotides at positions 54 and 55 of SEQ ID NO: 240. These specific nucleotides define the fusion junction. Preferably the fragment comprises or consists of at least about 30, at least about 40, at least about 50, at least about 60, at least about 70, at least about 80, at least about 90, at least about 100 or all contiguous nucleotides of SEQ ID NO: 240.

The APP-NRG1 fusion polynucleotide may be a variant of one of these sequences. In an embodiment, the APP-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 240. In another embodiment, the APP-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 240. Sequence identity is typically measured over the full length of the non-variant sequence.

Methods for measuring sequence identity are discussed in more detail below. In another embodiment, the APP-NRG1 fusion polynucleotide comprises or consists of a fragment of at least 20 contiguous nucleotides of a variant sequence having at least about 70% sequence identity or any of the other % sequence identities listed above to SEQ ID NO: 240, including at least the nucleotides at positions 54 and 55 of SEQ ID NO: 240. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 240.

In another disclosed embodiment, the NRG1 fusion polynucleotide comprises or consists of a WRN-NRG1 fusion polynucleotide. The WRN-NRG1 fusion polynucleotide preferably comprises or consists of the sequence of SEQ ID NO: 260. In another embodiment, the WRN-NRG1 fusion polynucleotide comprises or consists of a fragment of at least 20 contiguous nucleotides of SEQ ID NO: 260, including at least the nucleotides at positions 96 and 97 of SEQ ID NO: 260. These specific nucleotides define the fusion junction. Preferably the fragment comprises or consists of at least about 30, at least about 40, at least about 50, at least about 60, at least about 70, at least about 80, at least about 90, at least about 100 or all contiguous nucleotides of SEQ ID NO: 260.

The WRN-NRG1 fusion polynucleotide may be a variant of one of these sequences. In an embodiment, the WRN-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 260. In another embodiment, the WRN-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 260. Sequence identity is typically measured over the full length of the non-variant sequence. Methods for measuring sequence identity are discussed in more detail below. In another embodiment, the WRN-NRG1 fusion polynucleotide comprises or consists of a fragment of at least 20 contiguous nucleotides of a variant sequence having at least about 70% sequence identity or any of the other % sequence identities listed above to SEQ ID NO: 260, including at least the nucleotides at positions 96 and 97 of SEQ ID NO: 260. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 260.

In another disclosed embodiment, the NRG1 fusion polynucleotide comprises or consists of a DAAM1-NRG1 fusion polynucleotide. The DAAM1-NRG1 fusion polynucleotide preferably comprises or consists of the sequence of SEQ ID NO: 298. In another embodiment, the DAAM1-NRG1 fusion polynucleotide comprises or consists of a fragment of at least 20 contiguous nucleotides of SEQ ID NO: 298, including at least the nucleotides at positions 75 and 76 of SEQ ID NO: 298. These specific nucleotides define the fusion junction. Preferably the fragment comprises or consists of at least about 30, at least about 40, at least about 50, at least about 60, at least about 70, at least about 80, at least about 90, at least about 100 or all contiguous nucleotides of SEQ ID NO: 298.

The DAAM1-NRG1 fusion polynucleotide may be a variant of one of these sequences. In an embodiment, the DAAM1-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 298. In another embodiment, the DAAM1-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 298. Sequence identity is typically measured over the full length of the non-variant sequence. Methods for measuring sequence identity are discussed in more detail below. In another embodiment, the DAAM1-NRG1 fusion polynucleotide comprises or consists of a fragment of at least 20 contiguous nucleotides of a variant sequence having at least about 70% sequence identity or any of the other % sequence identities listed above to SEQ ID NO: 298, including at least the nucleotides at positions 75 and 76 of SEQ ID NO: 298. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 298.

In another disclosed embodiment, the NRG1 fusion polynucleotide comprises or consists of a ASPH-NRG1 fusion polynucleotide. The ASPH-NRG1 fusion polynucleotide preferably comprises or consists of the sequence of SEQ ID NO: 325. In another embodiment, the ASPH-NRG1 fusion polynucleotide comprises or consists of a fragment of at least 20 contiguous nucleotides of SEQ ID NO: 325, including at least the nucleotides at positions 75 and 76 of SEQ ID NO: 325. These specific nucleotides define the fusion junction. Preferably the fragment comprises or consists of at least about 30, at least about 40, at least about 50, at least about 60, at least about 70, at least about 80, at least about 90, at least about 100 or all contiguous nucleotides of SEQ ID NO: 325.

The ASPH-NRG1 fusion polynucleotide may be a variant of one of these sequences. In an embodiment, the ASPH-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 325. In another embodiment, the ASPH-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 325. Sequence identity is typically measured over the full length of the non-variant sequence. Methods for measuring sequence identity are discussed in more detail below. In another embodiment, the ASPH-NRG1 fusion polynucleotide comprises or consists of a fragment of at least 20 contiguous nucleotides of a variant sequence having at least about 70% sequence identity or any of the other % sequence identities listed above to SEQ ID NO: 325, including at least the nucleotides at positions 75 and 76 of SEQ ID NO: 325. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 325.

In another disclosed embodiment, the NRG1 fusion polynucleotide comprises or consists of a NOTCH2-NRG1 fusion polynucleotide. The NOTCH2-NRG1 fusion polynucleotide preferably comprises or consists of the sequence of SEQ ID NO: 353. In another embodiment, the NOTCH2-NRG1 fusion polynucleotide comprises or consists of a fragment of at least 20 contiguous nucleotides of SEQ ID NO: 353, including at least the nucleotides at positions 75 and 76 of SEQ ID NO: 353. These specific nucleotides define the fusion junction. Preferably the fragment comprises or consists of at least about 30, at least about 40, at least about 50, at least about 60, at least about 70, at least about 80, at least about 90, at least about 100 or all contiguous nucleotides of SEQ ID NO: 353.

The NOTCH2-NRG1 fusion polynucleotide may be a variant of one of these sequences. In an embodiment, the NOTCH2-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 353. In another embodiment, the NOTCH2-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 353. Sequence identity is typically measured over the full length of the non-variant sequence. Methods for measuring sequence identity are discussed in more detail below. In another embodiment, the NOTCH2-NRG1 fusion polynucleotide comprises or consists of a fragment of at least 20 contiguous nucleotides of a variant sequence having at least about 70% sequence identity or any of the other % sequence identities listed above to SEQ ID NO: 353, including at least the nucleotides at positions 75 and 76 of SEQ ID NO: 353. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 353.

In another disclosed embodiment, the NRG1 fusion polynucleotide comprises or consists of a CD74-NRG1 fusion polynucleotide. The CD74-NRG1 fusion polynucleotide preferably comprises or consists of the sequence of SEQ ID NO: 364. In another embodiment, the CD74-NRG1 fusion polynucleotide comprises or consists of a fragment of at least 20 contiguous nucleotides of SEQ ID NO: 364, including at least the nucleotides at positions 75 and 76 of SEQ ID NO: 364. These specific nucleotides define the fusion junction. Preferably the fragment comprises or consists of at least about 30, at least about 40, at least about 50, at least about 60, at least about 70, at least about 80, at least about 90, at least about 100 or all contiguous nucleotides of SEQ ID NO: 364.

The CD74-NRG1 fusion polynucleotide may be a variant of one of these sequences. In an embodiment, the CD74-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 364. In another embodiment, the CD74-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 364. Sequence identity is typically measured over the full length of the non-variant sequence. Methods for measuring sequence identity are discussed in more detail below. In another embodiment, the CD74-NRG1 fusion polynucleotide comprises or consists of a fragment of at least 20 contiguous nucleotides of a variant sequence having at least about 70% sequence identity or any of the other % sequence identities listed above to SEQ ID NO: 364, including at least the nucleotides at positions 75 and 76 of SEQ ID NO: 364. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 364.

In another disclosed embodiment, the NRG1 fusion polynucleotide comprises or consists of a SDC4-NRG1 fusion polynucleotide. The SDC4-NRG1 fusion polynucleotide preferably comprises or consists of the sequence of SEQ ID NO: 376. In another embodiment, the SDC4-NRG1 fusion polynucleotide comprises or consists of a fragment of at least 20 contiguous nucleotides of SEQ ID NO: 376, including at least the nucleotides at positions 75 and 76 of SEQ ID NO: 376. These specific nucleotides define the fusion junction. Preferably the fragment comprises or consists of at least about 30, at least about 40, at least about 50, at least about 60, at least about 70, at least about 80, at least about 90, at least about 100 or all contiguous nucleotides of SEQ ID NO: 376.

The SDC4-NRG1 fusion polynucleotide may be a variant of one of these sequences. In an embodiment, the SDC4-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 376. In another embodiment, the SDC4-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 376. Sequence identity is typically measured over the full length of the non-variant sequence. Methods for measuring sequence identity are discussed in more detail below. In another embodiment, the SDC4-NRG1 fusion polynucleotide comprises or consists of a fragment of at least 20 contiguous nucleotides of a variant sequence having at least about 70% sequence identity or any of the other % sequence identities listed above to SEQ ID NO: 376, including at least the nucleotides at positions 75 and 76 of SEQ ID NO: 376. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 376.

In another disclosed embodiment, the NRG1 fusion polynucleotide comprises or consists of a SDC4-NRG1 fusion polynucleotide of SEQ ID NO: 468. In another embodiment, the SDC4-NRG1 fusion polynucleotide comprises or consists of a fragment of at least 20 contiguous nucleotides of SEQ ID NO: 468, including at least the nucleotides at positions 75 and 76 of SEQ ID NO: 468. These specific nucleotides define the fusion junction. Preferably the fragment comprises or consists of at least about 30, at least about 40, at least about 50, at least about 60, at least about 70, at least about 80, at least about 90, at least about 100 or all contiguous nucleotides of SEQ ID NO: 468.

The SDC4-NRG1 fusion polynucleotide may be a variant of one of these sequences. In an embodiment, the SDC4-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 468. In another embodiment, the SDC4-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 468. Sequence identity is typically measured over the full length of the non-variant sequence. Methods for measuring sequence identity are discussed in more detail below. In another embodiment, the SDC4-NRG1 fusion polynucleotide comprises or consists of a fragment of at least 20 contiguous nucleotides of a variant sequence having at least about 70% sequence identity or any of the other % sequence identities listed above to SEQ ID NO: 468, including at least the nucleotides at positions 75 and 76 of SEQ ID NO: 468. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 468.

In another disclosed embodiment, the NRG1 fusion polynucleotide comprises or consists of a SLC4A4-NRG1 fusion polynucleotide. The SLC4A4-NRG1 fusion polynucleotide preferably comprises or consists of the sequence of SEQ ID NO: 384. In another embodiment, the SLC4A4-NRG1 fusion polynucleotide comprises or consists of a fragment of at least 20 contiguous nucleotides of SEQ ID NO: 384, including at least the nucleotides at positions 75 and 76 of SEQ ID NO: 384. These specific nucleotides define the fusion junction. Preferably the fragment comprises or consists of at least about 30, at least about 40, at least about 50, at least about 60, at least about 70, at least about 80, at least about 90, at least about 100 or all contiguous nucleotides of SEQ ID NO: 384.

The SLC4A4-NRG1 fusion polynucleotide may be a variant of one of these sequences. In an embodiment, the SLC4A4-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 384. In another embodiment, the SLC4A4-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 384.

Sequence identity is typically measured over the full length of the non-variant sequence. Methods for measuring sequence identity are discussed in more detail below. In another embodiment, the SLC4A4-NRG1 fusion polynucleotide comprises or consists of a fragment of at least 20 contiguous nucleotides of a variant sequence having at least about 70% sequence identity or any of the other % sequence identities listed above to SEQ ID NO: 384, including at least the nucleotides at positions 75 and 76 of SEQ ID NO: 384. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 384.

In another disclosed embodiment, the NRG1 fusion polynucleotide comprises or consists of a ZFAT-NRG1 fusion polynucleotide. The ZFAT-NRG1 fusion polynucleotide preferably comprises or consists of the sequence of SEQ ID NO: 413. In another embodiment, the ZFAT-NRG1 fusion polynucleotide comprises or consists of a fragment of at least 20 contiguous nucleotides of SEQ ID NO: 413, including at least the nucleotides at positions 75 and 76 of SEQ ID NO: 413. These specific nucleotides define the fusion junction. Preferably the fragment comprises or consists of at least about 30, at least about 40, at least about 50, at least about 60, at least about 70, at least about 80, at least about 90, at least about 100 or all contiguous nucleotides of SEQ ID NO: 413.

The ZFAT-NRG1 fusion polynucleotide may be a variant of one of these sequences. In an embodiment, the ZFAT-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 413. In another embodiment, the ZFAT-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 413. Sequence identity is typically measured over the full length of the non-variant sequence. Methods for measuring sequence identity are discussed in more detail below. In another embodiment, the ZFAT-NRG1 fusion polynucleotide comprises or consists of a fragment of at least 20 contiguous nucleotides of a variant sequence having at least about 70% sequence identity or any of the other % sequence identities listed above to SEQ ID NO: 413, including at least the nucleotides at positions 75 and 76 of SEQ ID NO: 413. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 413.

In another disclosed embodiment, the NRG1 fusion polynucleotide comprises or consists of a DSCAML1-NRG1 fusion polynucleotide. The DSCAML1-NRG1 fusion polynucleotide preferably comprises or consists of the sequence of SEQ ID NO: 432. In another embodiment, the DSCAML1-NRG1 fusion polynucleotide comprises or consists of a fragment of at least 20 contiguous nucleotides of SEQ ID NO: 432, including at least the nucleotides at positions 75 and 76 of SEQ ID NO: 432. These specific nucleotides define the fusion junction. Preferably the fragment comprises or consists of at least about 30, at least about 40, at least about 50, at least about 60, at least about 70, at least about 80, at least about 90, at least about 100 or all contiguous nucleotides of SEQ ID NO: 432.

The DSCAML1-NRG1 fusion polynucleotide may be a variant of one of these sequences. In an embodiment, the DSCAML1-NRG1 fusion polynucleotide may comprise one or more (i.e. 1, 2, 3, 4, 5, 6, 7, 8, 9, 10 or more) point mutations that add, delete or substitute any of the nucleotides of SEQ ID NO: 432. In another embodiment, the DSCAML1-NRG1 fusion polynucleotide comprises or consists of a sequence having at least about 70%, at least about 80%, at least about 85%, at least about 90%, at least about 95%, at least about 97%, at least about 98% or at least about 99% sequence identity to SEQ ID NO: 432. Sequence identity is typically measured over the full length of the non-variant sequence. Methods for measuring sequence identity are discussed in more detail below. In another embodiment, the DSCAML1-NRG1 fusion polynucleotide comprises or consists of a fragment of at least 20 contiguous nucleotides of a variant sequence having at least about 70% sequence identity or any of the other % sequence identities listed above to SEQ ID NO: 432, including at least the nucleotides at positions 75 and 76 of SEQ ID NO: 432. Preferably the fragment comprises or consists of at least about 30, at least about 40, at least about 50, at least about 60, at least about 70, at least about 80, at least about 90, at least about 100 or more contiguous nucleotides of a variant of SEQ ID NO: 432.

The NRG1 fusion polynucleotide most preferably comprises or consists of the sequence of SEQ ID NO: 19, 27, 41, 62, 74, 83, 102, 109, 120, 146, 154, 176, 201, 217, 225, 240, 260, 298, 325, 353, 364, 376, 384, 413, 432, 468 or 469.

### VAPB-NRG1 Fusion Data

In a disclosed embodiment, there is provided an isolated, patient-derived VAPB-NRG1 polynucleotide fusion sequence according to SEQ ID NO: 19 (see also “Sequence Information” herein). This sequence revealed the presence of a fusion junction between exon 1 of VAPB and exon 2 of NRG1. The underlined sequence, nucleotides 1-43, corresponds to a portion of exon 1 of the gene coding for VAPB (NM_004738.4). Nucleotides 44-94 correspond to a portion of the gene coding for NRG1. The sequence was obtained by way of nucleotide extraction from a formalin-fixed paraffin-embedded tissue sample, but is indicative for the type of NRG1 fusion sequences that can be obtained from subjects.

(SEQ ID NO: 19)
CAGGTCCTGAGCCTCGAGCCGCAGCACGAGCTCAAATTCCGAGCCTT
GCCTCCCCGATTGAAAGAGATGAAAAGCCAGGAATCGGCTGCAGGTT

### CADM1-NRG1 Fusion Data

In a disclosed embodiment, there is provided an isolated, patient-derived CADM1-NRG1 polynucleotide fusion sequence according to SEQ ID NO: 27 (see also “Sequence Information” herein). This sequence revealed the presence of a fusion junction between exon 7 of CADM1 and exon 6 of NRG1. The underlined sequence, nucleotides 1-53, corresponds to a portion of exon 7 of the gene coding for CADM1 (NM_001301045.1). Nucleotides 54-85 correspond to a portion of the gene coding for NRG1 (NM_001159999.3). The sequence was obtained by way of nucleotide extraction from a formalin-fixed paraffin-embedded tissue sample, but is indicative for the type of NRG1 fusion sequences that can be obtained from subjects.

(SEQ ID NO: 27)
AGCTTCAAACATAGTGGGGAAAGCTCACTCGGATTATATGCTGTATG
TATACGCTACATCTACATCCACCACTGGGACAAGCCAT

### CD44-NRG1 Fusion Data

In a disclosed embodiment, there is provided an isolated, patient-derived CD44-NRG1 polynucleotide fusion sequence according to SEQ ID NO: 41 (see also “Sequence Information” herein). This sequence revealed the presence of a fusion junction between exon 5 of CD44 and exon 2 of NRG1. The underlined sequence, nucleotides 1-52, corresponds to a portion from exon 5 of the CD44 gene (NM_000610). Nucleotides 53-110 correspond to a portion of exon 2 of the gene coding for NRG1. The sequence was obtained by way of nucleotide extraction from a formalin-fixed paraffin-embedded tissue sample, but is indicative for the type of NRG1 fusion sequences that can be obtained from subjects.

(SEQ ID NO: 41)
GACGAAGACAGTCCCTGGATCACCGACAGCACAGACAGAATCCCTGC
TACCACCTTGCCTCCCCGATTGAAAGAGATGAAAAGCCAGGAATCGG
CTGCAGGTTCCAAACT

### SLC3A2-NRG1 Fusion Data

In a disclosed embodiment, there is provided an isolated, patient-derived VAPB-NRG1 polynucleotide fusion sequence according to SEQ ID NO: 62 (see also “Sequence Information” herein). This sequence revealed the presence of a fusion junction between exon 1 of transcript version 6 of SLC3A2 and exon 5 of NRG1. The underlined sequence, nucleotides 1-53, corresponds to a portion of exon 1 of the gene coding for transcript version 6 of SLC3A2 (NM_001013251). The remaining 35 nucleotides, nucleotides 54-88, correspond to a portion of exon 5 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 62)
CCGCATCGGCGACCTTCAGGCCTTCCAGGGCCACGGCGCGGGCAACC
TGGCGGCATCTACATCTACATCCACCACTGGGACAAGCCAT

### VTCN1-NRG1 Fusion Data

In a disclosed embodiment, there is provided an isolated, patient-derived VTCN1-NRG1 polynucleotide fusion sequence according to SEQ ID NO: 74 (see also “Sequence Information” herein). This sequence revealed the presence of a fusion junction between exon 2 of VTCN1 and exon 5 of NRG1. The underlined sequence, nucleotides 1-65, corresponds to a portion of exon 2 of the gene coding for VTCN1 (NM_024626.4). The remaining 35 nucleotides, nucleotides 66-93, correspond to a portion of exon 2 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 74)
CATAATTAGCATCATCATTATTCTGGCTGGAGCAATTGCACTCATCA
TTGGCTTTGGTATTTCAGCCTTGCCTCCCCGATTGAAAGAGATGAA

### CDH1-NRG1 Fusion Data

In a disclosed embodiment, there is provided an isolated, patient-derived VAPB-NRG1 polynucleotide fusion sequence according to SEQ ID NO: 83 (see also “Sequence Information” herein). This sequence revealed the presence of a fusion junction between exon 11 of CDH1 and exon 2 of NRG1. The underlined sequence, nucleotides 1-119, corresponds to a portion of exon 11 of the gene coding for CDH1 (NM_001317185.2). The remaining 35 nucleotides, nucleotides 120-148, correspond to a portion of exon 2 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 83)
CTGGCTGGAGATTAATCCGGACACTGGTGCCATTTCCACTCGGGCTG
AGCTGGACAGGGAGGATTTTGAGCACGTGAAGAACAGCACGTACACA
GCCCTAATCATAGCTACAGACAATGCCTTGCCTCCCCGATTGAAAGA
GATGAAA

### CXADR-NRG1 Fusion Data

In a disclosed embodiment, there is provided an isolated, patient-derived CXADR-NRG1 polynucleotide fusion sequence according to SEQ ID NO: 102 see also “Sequence Information” herein). This sequence revealed the presence of a fusion junction between exon 1 of CXADR and exon 2 of NRG1. The underlined sequence, nucleotides 1-43 corresponds to a portion of exon 1 of the gene coding for CXADR (NM_001207063.2). The remaining 58 nucleotides, nucleotides 44-101, correspond to a portion of exon 2 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 102)
ATGGCGCTCCTGCTGTGCTTCGTGCTCCTGTGCGGAGTAGTGGCCTT
GCCTCCCCGATTGAAAGAGATGAAAAGCCAGGAATCGGCTGCAGGTT
CCAAACT.

### GTF2E2-NRG1 Fusion Data

In a disclosed embodiment, there is provided an isolated, patient-derived GTF2E2-NRG1 polynucleotide fusion sequence according to SEQ ID NO: 109 (see also “Sequence Information” herein). This sequence revealed the presence of a fusion junction between exon 2 of GTF2E2 and exon 2 of NRG1. The underlined sequence, nucleotides 1-141 corresponds to a portion of exon 2 of the gene coding for GTF2E2 (NM_002095.6). The remaining 127 nucleotides, nucleotides 142-268, correspond to a portion of exon 2 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 109)
GGGAGCTGTTCAAAAAACGAGCTCTTTCTACTCCTGTAGTAGAAAAACGT
TCAGCATCTTCTGAGTCATCATCATCATCGTCAAAGAAGAAGAAAACAAA
GGTAGAACATGGAGGATCGTCAGGCTCTAAACAAAATTCTGCCTTGCCTC
CCCGATTGAAAGAGATGAAAAGCCAGGAATCGGCTGCAGGTTCCAAACTA
GTCCTTCGGTGTGAAACCAGTTCTGAATACTCCTCTCTCAGATTCAAGTG
GTTCAAGAATGGGAATGA.

### CSMD1-NRG1 Fusion Data

In a disclosed embodiment, there is provided an isolated, patient-derived CSMD1-NRG1 polynucleotide fusion sequence according to SEQ ID NO: 120 (see also “Sequence Information” herein). This sequence revealed the presence of a fusion junction between exon 23 of CSMD1 and exon 6 of NRG1. The underlined sequence, nucleotides 1-88 corresponds to a portion of exon 23 of the gene coding for CSMD1 (NM_033225.6). The remaining 62 nucleotides, nucleotides 89-150, correspond to a portion of exon 6 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 120)
ATCCTAAACAGCACATCCAATCACCTGTGGCTAGAGTTCAACACCAATGG
ATCTGACACCGACCAAGGTTTTCAACTCACCTATACCACTACATCTACAT
CCACCACTGGGACAAGCCATCTTGTAAAATGTGCGGAGAAGGAGAAAACT.

### PTN-NRG1 Fusion Data

In a disclosed embodiment, there is provided an isolated, patient-derived PTN-NRG1 polynucleotide fusion sequence according to SEQ ID NO: 146 (see also “Sequence Information” herein). This sequence revealed the presence of a fusion junction between exon 4 of PTN and exon 2 of NRG1. The underlined sequence, nucleotides 1-102 corresponds to a portion of exon 4 of the gene coding for PTN (NM_001321386.2). The remaining 103 nucleotides, nucleotides 103-205, correspond to a portion of exon 2 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 146)
CCAGAACTGGAAGTCTGAAGCGAGCCCTGCACAATGCCGAATGCCAGAAG
ACTGTCACCATCTCCAAGCCCTGTGGCAAACTGACCAAGCCCAAACCTCA
AGCCTTGCCTCCCCGATTGAAAGAGATGAAAAGCCAGGAATCGGCTGCAG
GTTCCAAACTAGTCCTTCGGTGTGAAACCAGTTCTGAATACTCCTCTCTC
AGATT.

### ST14-NRG1 Fusion Data

In a disclosed embodiment, there is provided an isolated, patient-derived ST14-NRG1 polynucleotide fusion sequence according to SEQ ID NO: 154 (see also “Sequence Information” herein). This sequence revealed the presence of a fusion junction between exon 11 of ST14 and exon 6 of NRG1. The underlined sequence, nucleotides 1-95 corresponds to a portion of exon 11 of the gene coding for ST14 (NM_021978.4). The remaining 87 nucleotides, nucleotides 96-182, correspond to a portion of exon 6 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 154)
CAACAGCAACAAGATCACAGTTCGCTTCCACTCAGATCAGTCCTACACCG
ACACCGGCTTCTTAGCTGAATACCTCTCCTACGACTCCAGTGACCCTACA
TCTACATCCACCACTGGGACAAGCCATCTTGTAAAATGTGCGGAGAAGGA
GAAAACTTTCTGTGTGAATGGAGGGGAGTGCT.

### THBS1-NRG1 Fusion Data

In a disclosed embodiment, there is provided an isolated, patient-derived THBS1-NRG1 polynucleotide fusion sequence according to SEQ ID NO: 176 (see also “Sequence Information” herein). This sequence revealed the presence of a fusion junction between exon 9 of THBS1 and exon 6 of NRG1. The underlined sequence, nucleotides 1-56 corresponds to a portion of exon 9 of the gene coding for THBS1 (NM_003246.4). The remaining 89 nucleotides, nucleotides 90-145, correspond to a portion of exon 6 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 176)
ACCCTGTGAAGGCGAAGCGCGGGAGACCAAAGCCTGCAAGAAAGACGCCT
GCCCCACTACATCTACATCCACCACTGGGACAAGCCATCTTGTAAAATGT
GCGGAGAAGGAGAAAACTTTCTGTGTGAATGGAGGGGAGTGCTTC.

### AGRN-NRG1 Fusion Data

In a disclosed embodiment, there is provided an isolated, patient-derived AGRN-NRG1 polynucleotide fusion sequence according to SEQ ID NO: 201 (see also “Sequence Information” herein). This sequence revealed the presence of a fusion junction between exon 12 of AGRN and exon 6 of NRG1. The underlined sequence, nucleotides 1-106 corresponds to a portion of exon 12 of the gene coding for AGRN (NM_001305275.2). The remaining 101 nucleotides, nucleotides 107-207, correspond to a portion of exon 6 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 201)
GTGTGCGGCTCAGATGGGGTCACCTACAGCACCGAGTGTGAGCTGAAGAA
GGCCAGGTGTGAGTCACAGCGAGGGCTCTACGTAGCGGCCCAGGGAGCCT
GCCGAGCTACATCTACATCCACCACTGGGACAAGCCATCTTGTAAAATGT
GCGGAGAAGGAGAAAACTTTCTGTGTGAATGGAGGGGAGTGCTTCATGGT
GAAAGAC.

### PVALB-NRG1 Fusion Data

In a disclosed embodiment, there is provided an isolated, patient-derived PVALB-NRG1 polynucleotide fusion sequence according to SEQ ID NO: 217 (see also “Sequence Information” herein). This sequence revealed the presence of a fusion junction between exon 4 of PVALB and exon 6 of NRG1. The underlined sequence, nucleotides 1-102 corresponds to a portion of exon 4 of the gene coding for PVALB (NM_002854.3). The remaining 125 nucleotides, nucleotides 103-227, correspond to a portion of exon 6 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 217)
TAAAAGGCTTCTCCCCAGATGCCAGAGACCTGTCTGCTAAAGAAACCAAG
ATGCTGATGGCTGCTGGAGACAAAGATGGGGACGGCAAAATTGGGGTTGA
CGCTACATCTACATCCACCACTGGGACAAGCCATCTTGTAAAATGTGCGG
AGAAGGAGAAAACTTTCTGTGTGAATGGAGGGGAGTGCTTCATGGTGAAA
GACCTTTCAAACCCCTCGAGATACTTG.

### Further SLC3A2-NRG1 Fusion Data

In a disclosed embodiment, there is provided an isolated, patient-derived SLC3A2-NRG1 polynucleotide fusion sequence according to SEQ ID NO: 225 (see also “Sequence Information” herein). This sequence revealed the presence of a fusion junction between exon 2 of of transcript version 3 of SLC3A2 and exon 6 of NRG1. The underlined sequence, nucleotides 1-93, corresponds to a portion of exon 2 of the gene coding for transcript version 3 of SLC3A2 (NM_002394.6). The remaining 28 nucleotides, nucleotides 94-121, correspond to a portion of exon 6 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 225)
AGTTGGGGTCTCACTGTGTTGCCCAGACTGGTCTCGAACTCTTGGCCTCA
GGTGATCCTCTTCCCTCAGCTTCCCAGAATGCCGAGATGATAGCTACATC
TACATCCACCACTGGGACAAG.

### APP-NRG1 Fusion Data

In a disclosed embodiment, there is provided an isolated, patient-derived APP-NRG1 polynucleotide fusion sequence according to SEQ ID NO: 240 (see also “Sequence Information” herein). This sequence revealed the presence of a fusion junction between exon 14 of APP and exon 6 of NRG1. The underlined sequence, nucleotides 1-54 corresponds to a portion of exon 14 of the gene coding for APP (NM_001136130.3). The remaining 87 nucleotides, nucleotides 55-141, correspond to a portion of exon 6 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 240)
TTGAGCCTGTTGATGCCCGCCCTGCTGCCGACCGAGGACTGACCACTCGA
CCAGCTACATCTACATCCACCACTGGGACAAGCCATCTTGTAAAATGTGC
GGAGAAGGAGAAAACTTTCTGTGTGAATGGAGGGGAGTGCT.

### WRN-NRG1 Fusion Data

In a disclosed embodiment, there is provided an isolated, patient-derived WRN-NRG1 polynucleotide fusion sequence according to SEQ ID NO: 260 (see also “Sequence Information” herein). This sequence revealed the presence of a fusion junction between exon 33 of WRN and exon 6 of NRG1. The underlined sequence, nucleotides 1-96 corresponds to a portion of exon 33 of the gene coding for WRN (NM_000553.6). The remaining 87 nucleotides, nucleotides 97-182, correspond to a portion of exon 6 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 260)
AAGCTGGCTGCCCCCTTGATTTGGAGCGAGCAGGCCTGACTCCAGAGGTT
CAGAAGATTATTGCTGATGTTATCCGAAACCCTCCCGTCAACTCAGCTAC
ATCTACATCCACCACTGGGACAAGCCATCTTGTAAAATGTGCGGAGAAGG
AGAAAACTTTCTGTGTGAATGGAGGGGAGTGC.

### DAAM1-NRG1 Fusion Data

In a disclosed embodiment, there is provided an isolated, patient-derived DAAM1-NRG1 polynucleotide fusion sequence according to SEQ ID NO: 298 (see also “Sequence Information” herein). This sequence revealed the presence of a fusion junction between exon 1 of DAAM1 and exon 1 of NRG1. The underlined sequence, nucleotides 1-75 corresponds to a portion of exon 1 of the gene coding for DAAM1 (NM_001270520.2). The remaining nucleotides, nucleotides 76-150, correspond to a portion of exon 1 of the NRG1 gene. The sequence was obtained by way of nucleotide extraction from a formalin-fixed paraffin-embedded tissue sample, but is indicative for the type of NRG1 fusion sequences that can be obtained from subjects. Due to the position of the fusion junction, which is located in the 5′UTR of both DAAM1 as well as NRG1, no fusion polypeptide is expected to be formed from this particular fusion.

(SEQ ID NO: 298)
GAAGGAAACTGTTTAACCGGATCCCATTGTACCCAGAGTGCAGAGCCGCC
TTTCCAGCATGCAGGGGCTGCTCAGGACAGAGAGGGAGGAGGCGCGCGGG
GACGGGGACGCCCAGGAGGACCCACTCGCGGGTCCCGCTCCGCTCCGGCA.

### ASPH-NRG1 Fusion Data

In a disclosed embodiment, there is provided an isolated, patient-derived ASPH-NRG1 polynucleotide fusion sequence according to SEQ ID NO: 325 (see also “Sequence Information” herein). This sequence revealed the presence of a fusion junction between exon 22 of ASPH and exon 2 of NRG1. The underlined sequence corresponds to a portion of exon 22 of the gene coding for ASPH (NM_001164750.2). The remaining nucleotides correspond to a portion of exon 2 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 325)
AAGGTCTCTTCCTGCCTGAGGATGAAAACCTGAGGGAAAAAGGGGACTGG
AGCCAGTTCACGCTGTGGCAGCAAGCCTTGCCTCCCCGATTGAAAGAGAT
GAAAAGCCAGGAATCGGCTGCAGGTTCCAAACTAGTCCTTCGGTGTGAAA.

### NOTCH2-NRG1 Fusion Data

In a disclosed embodiment, there is provided an isolated, patient-derived NOTCH2-NRG1 polynucleotide fusion sequence according to SEQ ID NO: 353 (see also “Sequence Information” herein). This sequence revealed the presence of a fusion junction between exon 6 of NOTCH2 and exon 6 of NRG1. The underlined sequence corresponds to a portion of exon 6 of the gene coding for NOTCH2 (NM_024408.4). The remaining nucleotides correspond to a portion of exon 6 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 353)
CCTCCTGTACTCCAGGCTCCACCTGCATCGACCGTGTGGCCTCCTTCTCT
TGCATGTGCCCAGAGGGGAAGGCAGCTACATCTACATCCACCACTGGGAC
AAGCCATCTTGTAAAATGTGCGGAGAAGGAGAAAACTTTCTGTGTGAATG.

### CD74-NRG1 Fusion Data

In a disclosed embodiment, there is provided an isolated, patient-derived CD74-NRG1 polynucleotide fusion sequence according to SEQ ID NO: 364 (see also “Sequence Information” herein). This sequence revealed the presence of a fusion junction between exon 2 of CD74 and exon 2 of NRG1. The underlined sequence corresponds to a portion of exon 2 of the gene coding for CD74 (NM_001025159.3). The remaining nucleotides correspond to a portion of exon 2 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 364)
AGGGCCGGCTGGACAAACTGACAGTCACCTCCCAGAACCTGCAGCTGGAG
AACCTGCGCATGAAGCTTCCCAAGCCCTTGCCTCCCCGATTGAAAGAGAT
GAAAAGCCAGGAATCGGCTGCAGGTTCCAAACTAGTCCTTCGGTGTGAAA.

### SDC4-NRG1 Fusion Data

In a disclosed embodiment, there is provided an isolated, patient-derived SDC4-NRG1 polynucleotide fusion sequence according to SEQ ID NO: 376 (see also “Sequence Information” herein). This sequence revealed the presence of a fusion junction between exon 2 of SDC4 and exon 2 of NRG1. The underlined sequence corresponds to a portion of exon 2 the gene coding for SDC4 (NM_002999.4). The remaining nucleotides correspond to a portion of exon 2 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 376)
TACCAGACGATGAGGATGTAGTGGGGCCCGGGCAGGAATCTGATGACTTT
GAGCTGTCTGGCTCTGGAGATCTGGCCTTGCCTCCCCGATTGAAAGAGAT
GAAAAGCCAGGAATCGGCTGCAGGTTCCAAACTAGTCCTTCGGTGTGAAA.

In another disclosed embodiment, there is provided an isolated, patient-derived SDC4-NRG1 polynucleotide fusion sequence according to SEQ ID NO: 468 (see also “Sequence Information” herein). This sequence revealed the presence of a fusion junction between exon 4 of SDC4 and exon 2 of NRG1. The underlined sequence corresponds to a portion of exon 4 the gene coding for SDC4 (NM_002999.4). The remaining nucleotides correspond to a portion of exon 2 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 468)
ATGTGTCCAACAAGGTGTCAATGTCCAGCACTGTGCAGGGCAGCA
ACATCTTTGAGAGAACGGAGGTCCTGGCAGCCTTGCCTCCCCGAT
TGAAAGAGATGAAAAGCCAGGAATCGGCTGCAGGTTCCAAACTAG
TCCTTCGGTGTGAAA

### Further CD44-NRG1 Fusion Data

In a disclosed embodiment, there is provided an isolated, patient-derived CD44-NRG1 polynucleotide fusion sequence according to SEQ ID NO: 469 (see also “Sequence Information” herein). This sequence revealed the presence of a fusion junction between exon 5 of CD44 and exon 6 of NRG1. The underlined sequence corresponds to a portion of exon 5 of the gene coding for CD44 (NM_000610.4). The remaining nucleotides correspond to a portion of exon 6 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 469)
TTTCTACTGTACACCCCATCCCAGACGAAGACAGTCCCTGGATCA
CCGACAGCACAGACAGAATCCCTGCTACCACTACATCTACATCCA
CCACTGGGACAAGCCATCTTGTAAAATGTGCGGAGAAGGAGAAAA
CTTTCTGTGTGAATG

### SLC4A4-NRG1 Fusion Data

In a disclosed embodiment, there is provided an isolated, patient-derived SLC4A4-NRG1 polynucleotide fusion sequence according to SEQ ID NO: 384 (see also “Sequence Information” herein). This sequence revealed the presence of a fusion junction between exon 14 of SLC4A4 and exon 6 of NRG1. The underlined sequence, nucleotides 1-27 corresponds to a portion of exon 14 of the gene coding for SLC4A4 (NM_001098484.3). The remaining 28 nucleotides, nucleotides 28-55, correspond to a portion of exon 6 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 384)
ACTACCCCATCAACTCCAACTTCAAAGTGGGCTACAACACTCTCT
TTTCCTGTACCTGTGTGCCACCTGACCCAGCTACATCTACATCCA
CCACTGGGACAAGCCATCTTGTAAAATGTGCGGAGAAGGAGAAAA
CTTTCTGTGTGAATG

### ZFAT-NRG1 Fusion Data

In a disclosed embodiment, there is provided an isolated, patient-derived ZFAT-NRG1 polynucleotide fusion sequence according to SEQ ID NO: 413 (see also “Sequence Information” herein). This sequence revealed the presence of a fusion junction between exon 12 of ZFAT and exon 6 of NRG1. The underlined sequence corresponds to a portion of exon 12 of the gene coding for ZFAT (NM_020863.4). The remaining nucleotides correspond to a portion of exon 6 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 413)
ACAGGAAGCACCCTAATGAGGAGTATGCCAACGTGGGCACCGGGG
AGCTGGCAGCGGAGGTGCTCATCCAGCAAGCTACATCTACATCCA
CCACTGGGACAAGCCATCTTGTAAAATGTGCGGAGAAGGAGAAAA
CTTTCTGTGTGAATG

### DSCAML1-NRG1 Fusion Data

In a disclosed embodiment, there is provided an isolated, patient-derived DSCAML1-NRG1 polynucleotide fusion sequence according to SEQ ID NO: 432 (see also “Sequence Information” herein). This sequence revealed the presence of a fusion junction between exon 3 of DSCAML1 and exon 2 of NRG1. The underlined sequence corresponds to a portion of exon 3 of the gene coding for DSCAML1 (NM_020693.4). The remaining nucleotides correspond to a portion of exon 2 of the NRG1 gene. The sequence was obtained by way of nucleotide extraction from a formalin-fixed paraffin-embedded tissue sample, but is indicative for the type of NRG1 fusion sequences that can be obtained from subjects.

(SEQ ID NO: 432)
GCCTCATCCCCTCTTCAGTGCAGGAATATGTTAGCGTTGTATCTT
GGGAGAAAGACACAGTCTCCATCATCCCAGCCTTGCCTCCCCGAT
TGAAAGAGATGAAAAGCCAGGAATCGGCTGCAGGTTCCAAACTAG
TCCTTCGGTGTGAAA

### Fusion Polynucleotide Types

The NRG1 fusion polynucleotide may be any type of polynucleotide. The NRG1 fusion polynucleotide is preferably DNA or RNA. The NRG1 fusion polynucleotide may be genomic DNA or complementary DNA (cDNA). The NRG1 fusion polynucleotide may be messenger RNA (mRNA). The NRG1 fusion polynucleotide is most preferably mRNA or cDNA.

The NRG1 fusion polynucleotide may be any of the polynucleotide types discussed below with reference to the probes that may be used in the invention.

### Isolation

Preferably the method of the invention further comprises isolating one or more polynucleotide-containing components from the sample. The one or more polynucleotide-containing components are typically isolated from any cells or cellular material in the sample.

The one or more polynucleotide-containing components are preferably one or more of cell free polynucleotide, cell-free DNA (cfDNA), cell-free RNA (cfRNA), circulating tumor RNA (ctRNA), circulating tumor cells (CTCs), vesicular RNA, exosomes, extracellular vesicles and tumor-educated platelets. In such embodiments, the method preferably comprises determining the presence or absence of the NRG1 fusion polynucleotide in one or more polynucleotide-containing components isolated from the sample. Preferably, the method further comprises amplifying the NRG1 fusion polynucleotide if present in the one or more polynucleotide-containing components. Amplification is discussed in more detail below.

Methods for isolating one or more polynucleotide-containing components from liquid samples are known in the art, for instance using commercially available kits. Polynucleotides, cfDNA, cfRNA and ctRNA may be isolated using, for example, the QIAamp® circulating Nucleic Acid Kit (CNA) as described in Examples 3 and 4. CTCs may be isolated using the AdnaTest BreastCancerSelect kit (ADNAGEN, catalog #T-1-508) as described in Example 5 or using either CellSearch® (Menarini silicon biosystems, Italy). Vesicular RNA, exosomes and extracellular vesicles may be isolated using ExoRNeasy Maxi kit (cat. No. 77023, QIAGEN GmbH, Hilden, Germany) as described in Example 6. Tumor-educated platelets can be isolated as described in Example 7.

### Amplification

Preferably the method of the invention further comprises amplifying the NRG1 fusion polynucleotide if present in the sample. Preferably the method of the invention further comprises amplifying the NRG1 fusion polynucleotide if present in the one or more polynucleotide-containing components isolated from the sample. In such embodiments, the method preferably comprises determining the presence or absence of the NRG1 fusion polynucleotide in any amplification products.

Methods for amplifying polynucleotides are known in the art. Polynucleotides are typically amplified using polymerase chain reaction (PCR), such as digital PCR (dPCR), droplet digital PCR (ddPCR), anchored multiplex PCR, real time PCR or reverse transcription PCR (RT-PCR). dPCR is a modern technique that allows DNA, RNA and cDNA to be amplified and quantified. ddPCR carries out dPCR in water-oil emulsion droplets and can be conducted on DNA, RNA or cDNA using Bio-Rad's QX100™ or QX200™ Droplet Digital PCR system as described in Example 8. Anchored multiplex PCR allows the simultaneous amplification of multiple different polynucleotides and can be conducted using the Archer FusionPlex Solid Tumor kit (ArcherDX, cat. no. AB0005) as described in Example 9. Real time PCR monitors the real time amplification of polynucleotides. RT-PCR involves the use of the enzyme reverse transcriptase to convert RNA into cDNA and then the amplification of the cDNA using PCR. Methods for reverse transcription are well known in the art and reverse transcriptases are commercially available (e.g. Superscript® II reverse transcriptase (Invitrogen) and Affinity script (Agilent)).

PCR typically involves amplifying a DNA amplicon (either DNA from the sample or converted from RNA from the sample) using a heat-stable DNA polymerase and a pair of forward and reverse polynucleotide primers. Because the newly synthesized DNA strands can subsequently serve as additional templates for the same primer sequences, successive rounds of primer annealing, strand elongation, and dissociation produce rapid and highly specific amplification of the desired sequence. Many PCR methods are known to those of skill in the art and may be used in the methods of the invention. For example, DNA can be subjected to 20 to 40 cycles of amplification in a thermocycler as follows: 95° C. for 30 sec, 52° to 60° C. for 1 min, and 72° C. for 1 min, with a final extension step of 72° C. for 5 min. For another example, DNA can be subjected to 20 to 40 PCR cycles in a thermocycler at a denaturing temperature of 95° C. for 30 sec, followed by varying annealing temperatures ranging from 54° C. to 58° C. for 1 min, an extension step at 70° C. for 1 min, with a final extension step at 70° C. for 5 min. Heat stable DNA polymerases are commercially available (such as GoTaq G2 (Promega)).

Preferably the method of the invention further comprises amplifying the NRG1 fusion polynucleotide if present in the sample or amplifying the NRG1 fusion polynucleotide if present in the one or more polynucleotide-containing components isolated from the sample using a pair of forward and reverse polynucleotide primers. Preferably the method of the invention further comprises contacting the sample or the one or more polynucleotide-containing components isolated from the sample with a pair of forward and reverse polynucleotide primers and amplifying the NRG1 fusion polynucleotide if present.

The pair of forward and reverse polynucleotide primers preferably specifically hybridise to the NRG1 fusion polynucleotide. The pair of forward and reverse polynucleotide primers preferably specifically hybridise to the NRG1 fusion polynucleotide and amplify a part of or all of the NRG1 fusion polynucleotide. Each polynucleotide primer specifically hybridises to a target or selected sequence in the NRG1 fusion polynucleotide. The term “amplify” refers to the process of making multiple copies of a part of or all of the NRG1 fusion polynucleotide, such as cDNA, from a single polynucleotide or fewer polynucleotides.

The pair of forward and reverse polynucleotide primers may amplify a part of or all of the NRG1 fusion polynucleotide. The pair of polynucleotide primers may amplify NRG1 fusion polynucleotide amplicons of any size. The amplicons are preferably from about 30 to about 400 base pairs in size, such as from 40 to about 300, from about 60 to about 250, from about 70 to about 180 or from about 80 to about 150 base pair in length.

A primer “specifically hybridises” to its target sequence when it hybridises with preferential or high affinity to the target sequence but does not substantially hybridise, does not hybridise or hybridises with only low affinity to other polynucleotides, especially other sequences in the sample. Conditions that permit the hybridisation are well-known in the art (for example, Sambrook et al., 2001, Molecular Cloning: a laboratory manual, 3rd edition, Cold Spring Harbour Laboratory Press; and Current Protocols in Molecular Biology, Chapter 2, Ausubel et al., Eds., Greene Publishing and Wiley-Interscience, New York (1995)). Hybridisation can be carried out under low stringency conditions, for example in the presence of a buffered solution of 30 to 35% formamide, 1 M NaCl and 1% SDS (sodium dodecyl sulfate) at 37° C. followed by a 20 wash in from 1× (0.1650 M Na+) to 2× (0.33 M Na+) SSC (standard sodium citrate) at 50° C. Hybridisation can be carried out under moderate stringency conditions, for example in the presence of a buffer solution of 40 to 45% formamide, 1 M NaCl, and 1% SDS at 37° C., followed by a wash in from 0.5× (0.0825 M Na+) to 1× (0.1650 M Na+) SSC at 55° C. Hybridisation can be carried out under high stringency conditions, for example in the presence of a buffered solution of 50% formamide, 1 M NaCl, 1% SDS at 37° C., followed by a wash in 0.1× (0.0165 M Na+) SSC at 60° C. A primer “specifically hybridises” if it hybridises to its partner with a melting temperature (Tm) that is at least 2° C., such as at least 3° C., at least 4° C., at least 5° C., at least 6° C., at least 7° C., at least 8° C., at least 9° C. or at least 10° C., greater than its Tm for other polynucleotides. More preferably, the primer hybridises to its target sequence with a Tm that is at least 2° C., such as at least 3° C., at least 4° C., at least 5° C., at least 6° C., at least 7° C., at least 8° C., at least 9° C., at least 10° C., at least 20° C., at least 30° C. or at least 40° C., greater than its Tm for other polynucleotides. Preferably, the primer hybridises to its target with a Tm that is at least 2° C., such as at least 3° C., at least 4° C., at least 5° C., at least 6° C., at least 7° C., at least 8° C., at least 9° C., at least 10° C., at least 20° C., at least 30° C. or at least 40° C., greater than its Tm for a polynucleotide which differs from its target sequence by one or more nucleotides, such as by 1, 2, 3, 4 or 5 or more nucleotides. The primer typically hybridises to its target sequence with a Tm of at least 90° C., such as at least 92° C. or at least 95° C. Tm can be measured experimentally using known techniques, including the use of DNA microarrays, or can be calculated using publicly available Tm calculators, such as those available over the internet.

Over the entire length of its target sequence in the NRG1 fusion polynucleotide, a polynucleotide primer typically has at least about 80%, at least about 85%, at least about 90%, at least about 95%, at least about 97%, at least about 98% or at least about 99% sequence identity to the target sequence. The polynucleotide primer is most preferably complementary (100% sequence identity over the entire length) to its target sequence. In all instances herein, sequence identity is typically measured over the entire length of the non-variant/target sequence.

Methods of measuring homology based on sequence identity or identity are known in the art. “Percent (%) identity” as referring to nucleic acid or amino acid sequences is defined as the percentage of residues in a candidate sequence that are identical with the residues in a selected sequence, after aligning the sequences for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/based or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent sequence identity between two amino acid sequences or nucleic acid sequences may be determined using the Needleman and Wunsch algorithm for the alignment of two sequences. (Needleman, S. B. and Wunsch, C. D. (1970) J. Mol. Biol. 48, 443-453). The Needleman-Wunsch algorithm has been implemented in the computer program NEEDLE. For the purpose of this disclosure the NEEDLE program from the EMBOSS package is used to determine percent identity of amino acid and nucleic acid sequences (version 2.8.0 or higher, EMBOSS: The European Molecular Biology Open Software Suite (2000) Rice, P. Longden J. and Bleasby, A. Trends in Genetics 16, (6) pp 276-277, http://emboss.bioinformatics.nl/). For protein sequences, EBLOSUM62 is used for the substitution matrix. For DNA sequences, DNAFULL is used. The parameters used are a gap-open penalty of 10 and a gap extension penalty of 0.5.

After alignment by the program NEEDLE as described above the percentage of sequence identity between a query sequence and a sequence of the disclosure is calculated as follows: Number of corresponding positions in the alignment showing an identical amino acid or identical nucleotide in both sequences divided by the total length of the alignment after subtraction of the total number of gaps in the alignment.

The pair of forward and reverse polynucleotide primers most preferably specifically hybridise to the NRG1 fusion polynucleotide across the fusion junction. In other words, the forward polynucleotide primer preferably specifically hybridises to a target sequence on one side of the fusion junction and the reverse polynucleotide primer specifically hybridises to a target sequence on the other side of the fusion junction. Fusion junctions are defined above. The pair of forward and reverse polynucleotide primers preferably specifically hybridise to the NRG1 fusion polynucleotide across the fusion junction and amplify a part of or all of the NRG1 fusion polynucleotide comprising the fusion junction. The term “amplify” refers to the process of making multiple copies of a part of or all of the NRG1 fusion polynucleotide, such as cDNA, from a single polynucleotide or fewer polynucleotides.

Each polynucleotide primer may be any length. The forward polynucleotide primer is preferably at least about 12 nucleotides in length, such as about 12, 13, 14, 15, 16, 17, 18, 19, 20, 21, 22, 23, 24, 25, 26, 27, 28, 29 or 30 nucleotides in length. The reverse polynucleotide primer is preferably at least about 12 nucleotides in length, such as about 12, 13, 14, 15, 16, 17, 18, 19, 20, 21, 22, 23, 24, 25, 26, 27, 28, 29 or 30 nucleotides in length. Each polynucleotide primer is typically the same length as its corresponding target sequence in the NRG1 fusion polynucleotide. The aforementioned lengths of the forward and reverse polynucleotide primers equally apply to the target sequences to which they specifically hybridise.

The pair of forward and reverse polynucleotide primers may be DNA or RNA. The pair of forward and reverse polynucleotide primers are preferably DNA. The pair of forward and reverse polynucleotide primers may be single stranded or double stranded. The pair of forward and reverse polynucleotide primers are preferably single stranded. The pair of forward and reverse polynucleotide primers may comprise a detectable label as discussed below with reference to the probes that may be used in the method of the invention, but they typically do not.

Polynucleotide primers as described above may be manufactured using standard techniques. Custom primers having specific sequences are commercially available from various suppliers (such as Thermo Fisher Scientific or Biolegio).

### Sequencing

Preferably the method comprises determining the presence or absence of the NRG1 fusion polynucleotide in the sample by polynucleotide sequencing. Preferably the method of the invention comprises determining the presence or absence of the NRG1 fusion polynucleotide in the one or more polynucleotide-containing components isolated from the sample or any amplification products by polynucleotide sequencing. The method preferably further comprises polynucleotide sequencing any polynucleotides in the sample. The method preferably further comprises polynucleotide sequencing the one or more polynucleotide-containing components isolated from the sample or any amplification products.

Detection of the NRG1 fusion polynucleotide by polynucleotide sequencing indicates that the sample contains the NRG1 polynucleotide. A lack of detection of the NRG1 fusion polynucleotide by polynucleotide sequencing indicates that the sample does not contain the NRG1 fusion polynucleotide. Detection of multiple copies of the NRG1 fusion polynucleotide by polynucleotide sequencing indicates that the sample contains the NRG1 fusion polynucleotide. A lack of detection of multiple copies of the NRG1 fusion polynucleotide by polynucleotide sequencing indicates that the sample does not contain the NRG1 fusion polynucleotide. In all of these embodiments, the detection of the NRG1 fusion polynucleotide or lack of detection of the NRG1 fusion polynucleotide by polynucleotide sequencing includes detection or lack of detection of the fusion junction, the EGF-like domain of NRG1 or both the fusion junction and the EGF-like domain of NRG1.

Any method of polynucleotide sequencing may be used in the method of the invention. The polynucleotide sequencing is preferably next generation sequencing (NGS). The NGS may involve the use of an Illumina MiSeq machine and sample using the Archer Analysis 6.0 suite. Alternatively, the NGS may involve the use of nanopore sequencing technology from Oxford Nanopore Technologies Limited.

### Probes

Preferably the method comprises determining the presence or absence of the NRG1 fusion polynucleotide in the sample using a polynucleotide probe which specifically hybridises to the NRG1 fusion polynucleotide. Preferably the method of the invention comprises determining the presence or absence of the NRG1 fusion polynucleotide in the one or more polynucleotide-containing components isolated from the sample or any amplification products using a polynucleotide probe which specifically hybridises to the NRG1 fusion polynucleotide. The method preferably further comprises contacting the sample with a polynucleotide probe which specifically hybridises to the NRG1 fusion polynucleotide and thereby determining the presence or absence of the NRG1 fusion polynucleotide. The method preferably further comprises contacting the one or more polynucleotide-containing components isolated from the sample or any amplification products with a polynucleotide probe which specifically hybridises to the NRG1 fusion polynucleotide and thereby determining the presence or absence of the NRG1 fusion polynucleotide.

In all of these embodiments, the polynucleotide specifically hybridises to the fusion junction in the NRG1 fusion polynucleotide, the EGF-like domain of NRG1 in the NRG1 fusion polynucleotide or both the fusion junction and the EGF-like domain of NRG1 in the NRG1 fusion polynucleotide.

Detection of the NRG1 fusion polynucleotide by the polynucleotide probe indicates that the sample contains the NRG1 fusion polynucleotide. A lack of detection of the NRG1 fusion polynucleotide by the polynucleotide probe indicates that the sample does not contain the NRG1 fusion polynucleotide. Detection of multiple copies of the NRG1 fusion polynucleotide by the polynucleotide probe indicates that the sample contains the NRG1 polynucleotide. A lack of detection of multiple copies of the NRG1 fusion polynucleotide by the polynucleotide probe indicates that the sample does not contain the NRG1 fusion polynucleotide.

The polynucleotide probe typically specifically hybridises to a target sequence in the NRG1 polynucleotide. The target sequence preferably comprises the fusion junction in the NRG1 fusion polynucleotide, the EGF-like domain of NRG1 in the NRG1 fusion polynucleotide or both the fusion junction and the EGF-like domain of NRG1 in the NRG1 fusion polynucleotide. Specific hybridisation is defined above. Over the entire length of its target sequence in the NRG1 fusion polynucleotide, a polynucleotide probe typically has at least about 80%, at least about 85%, at least about 90%, at least about 95%, at least about 97%, at least about 98% or at least about 99% sequence identity to the target sequence. The polynucleotide probe is most preferably complementary (100% sequence identity over the entire length) to its target sequence. In all instances herein, sequence identity is typically measured over the entire length of the non-variant/target sequence. Methods for measuring sequence identity are described above.

The probe may be any polynucleotide. The polynucleotide, such as a nucleic acid, is a polymer comprising two or more nucleotides. The nucleotides can be naturally occurring or artificial. A nucleotide typically contains a nucleobase, a sugar and at least one linking group, such as a phosphate, 2′O-methyl, 2′ methoxy-ethyl, phosphoramidate, methylphosphonate or phosphorothioate group. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The sugar and the nucleobase together form a nucleoside. Preferred nucleosides include, but are not limited to, adenosine, guanosine, 5-methyluridine, uridine, cytidine, deoxyadenosine, deoxyguanosine, thymidine, deoxyuridine and deoxycytidine. The nucleosides are most preferably adenosine, guanosine, uridine and cytidine

The nucleotides are typically ribonucleotides or deoxyribonucleotides. The nucleotides are preferably deoxyribonucleotides. The nucleotides typically contain a monophosphate, diphosphate or triphosphate. Phosphates may be attached on the 5′ or 3′ side of a nucleotide.

Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate, cyclic adenosine monophosphate (CAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, 5-methyl-2′-deoxycytidine triphosphate, 5-hydroxymethyl-2′-deoxycytidine monophosphate, 5-hydroxymethyl-2′-deoxycytidine diphosphate and 5-hydroxymethyl-2′-deoxycytidine triphosphate. The nucleotides are preferably selected from AMP, UMP, GMP, CMP, dAMP, dTMP, dGMP and dCMP. The nucleotides are preferably selected from dAMP, dTMP, dGMP and dCMP.

The nucleotides may contain additional modifications. In particular, suitable modified nucleotides include, but are not limited to, 2′amino pyrimidines (such as 2′-amino cytidine and 2′-amino uridine), 2′-hyrdroxyl purines (such as, 2′-fluoro pyrimidines (such as 2′-fluorocytidine and 2′fluoro uridine), hydroxyl pyrimidines (such as 5′-α-P-borano uridine), 2′-O-methyl nucleotides (such as 2′-O-methyl adenosine, 2′-O-methyl guanosine, 2′-O-methyl cytidine and 2′-O-methyl uridine), 4′-thio pyrimidines (such as 4′-thio uridine and 4′-thio cytidine) and nucleotides have modifications of the nucleobase (such as 5-pentynyl-2′-deoxy uridine, 5-(3-aminopropyl)-uridine and 1,6-diaminohexyl-N-5-carbamoylmethyl uridine).

One or more nucleotides in the polynucleotide may be modified, for instance with a label or a tag. The label may be any suitable label which allows the polynucleotide to be detected.

Suitable labels include, but are not limited to, fluorescent molecules, radioisotopes, e.g. ¹²⁵I, ³⁵S, enzymes, antibodies, antigens, other polynucleotides and ligands such as biotin. Detectable labels are discussed in more detail below.

The nucleotides in the polynucleotide may be attached to each other in any manner. The nucleotides may be linked by phosphate, 2′O-methyl, 2′ methoxy-ethyl, phosphoramidate, methylphosphonate or phosphorothioate linkages. The nucleotides are typically attached by their sugar and phosphate groups as in nucleic acids. The nucleotides may be connected via their nucleobases as in pyrimidine dimers.

The polynucleotide can be a nucleic acid, such as deoxyribonucleic acid (DNA) or a ribonucleic acid (RNA). The polynucleotide may be any synthetic nucleic acid known in the art, such as peptide nucleic acid (PNA), glycerol nucleic acid (GNA), threose nucleic acid (TNA), locked nucleic acid (LNA), morpholino nucleic acid or other synthetic polymers with nucleotide side chains. The polynucleotide may comprise any of the nucleotides discussed above, including the modified nucleotides.

The polynucleotide probe may be DNA or RNA. The polynucleotide probe is preferably DNA.

The polynucleotide primer may be single stranded or double stranded. The polynucleotide primer is preferably single stranded.

Any of the polynucleotides discussed herein may be isolated, substantially isolated, purified or substantially purified. The polynucleotide is isolated or purified if it is completely free of any other components, such as buffer, other polynucleotides, virus material or cells. The polynucleotide is substantially isolated or substantially purified if it is only mixed with carriers or diluents, such as buffers or excipients, which will not interfere with its intended use, such as in a RT-PCR assay. The primer and probe polynucleotides are not naturally occurring.

The polynucleotide probe may be any length. The polynucleotide probe is preferably at least about 10 nucleotides in length, such as at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length. The polynucleotide probe is preferably from about 10 to about 40 nucleotides in length, such as from about 15 to about 35 nucleotides in length or from about 20 nucleotides to about 30 nucleotides in length. The polynucleotide probe is typically the same length as its corresponding target sequence in the NRG1 fusion polynucleotide. The aforementioned lengths of the polynucleotide probe equally apply to the target sequence in the NRG1 fusion polypeptide to which is specifically hybridises.

The polynucleotide probe is preferably a DNA probe, a TaqMan probe, a molecular beacon or a scorpion probe. DNA probes hybridise to the, typically complementary, target sequence and then can be detected, for instance using a detectable label. TaqMan probes are known in the art and are polynucleotides that have a fluorescent dye attached to the 5′ end and a quencher to the 3′ end. The polymerase used in PCR cleaves hybridised probes freeing the fluorescent dye from quenching such it can be detected. Molecular beacon probes are known in the art and are similar to TaqMan probes except (rather than using cleavage to separate the dye from the quencher) hybridisation to the target sequence separates the dye from the quencher. Scorpion probes are known the art and are similar to molecular beacons except the 3′ end also contains a sequence that is complementary to the extension product of the primer on the 5′ end which opens the probe on hybridisation and allows the dye to be detected. The polynucleotide probe is preferably a TaqMan probe. The polynucleotide probe is preferably a TaqMan probe and is used in any of the PCR methods discussed above. In disclosed embodiments, the polynucleotide probe hybridizes specifically to the NRG1 fusion polynucleotide to be identified by spanning the fusion junction of interest between A and B of the general formula 5′-A-B-3′.

The polynucleotide probe may be used to determine the presence or absence of the NRG1 fusion polynucleotide in a nucleotide-based Fluorescence In Situ Hybridization (FISH). FISH can be carried out on CTCs isolated from the sample.

The polynucleotide probes of the invention are preferably detectably-labelled. A “detectable label” means a chemical, biological, or other modification, including but not limited to fluorescence, mass, residue, dye, radioisotope, label, or tag modifications, etc., which allows the presence of the polynucleotide probe or polynucleotide of interest to be detected. Preferably, the detectable label is a visible label, a fluorescent dye, a quencher, a UV-detectable label, a chromogenic label, a radioactive label, an electrochemical label, a tag, an enzyme that produces a detectable label when in contact with a substrate specific for the enzyme or a molecular barcode. Suitable detectable labels are known in the art (Handbook of Molecular Probes and Research Reagents, 8th ed. (2002), Molecular Probes, Eugene, Oreg.; WO 2001/32783; U.S. Pat. Publ. Nos. US 2002-0081616, US 2002-0086985; and Lee et al., 1997, Nucleic Acids Research 25:2816-2822).

The detectable labels are preferably fluorescent molecules or dyes, such as fluorescein derivatives. Exemplary fluorescent dyes include water-soluble rhodamine dyes, fluoresceins, 4,7-dichlorofluoresceins, benzoxanthene dyes and energy transfer dyes. Suitable fluorescent molecules or dyes include, but are not limited to, 6-carboxyfluorescein (FAM), 6-Carboxyl-X-Rhodamine (ROX), 2′-chloro-7′phenyl-1,4-dichloro-6-carboxy-fluorescein (VIC®), Hexachloro-Fluorescein (HEX) and tetrachlorofluorescein (TET). A suitable quencher for use with these dyes in TaqMan, molecular beacon or scorpion probes is tetramethylrhodamine (TAMRA).

The detectable label is most preferably 6-Carboxyl-X-Rhodamine (ROX) or 2′-chloro-7′phenyl-1,4-dichloro-6-carboxy-fluorescein (VIC®).

Polynucleotide probes are also available from commercial sources (such as Thermo Fisher Scientific or Biolegio).

### Sample

The method of the invention comprises providing a liquid biopsy sample. The method may further comprise taking a liquid biopsy sample or taking a liquid biopsy sample from a subject. Suitable subjects are described in more detail below. Preferably, the samples contain an NRG1 fusion polynucleotide selected from the NRG1 fusion polynucleotides comprising or consisting of the sequences of SEQ ID NOs: 19, 27, 41, 62, 74, 83, 102, 109, 120, 146, 154, 176, 201, 217, 225, 240, 260, 298, 325, 353, 364, 376, 384, 413, 432, 468 or 469.

Preferably the sample is obtained from a subject which, with the exception of the NRG1 fusion polynucleotide, does not comprise any further oncogenic mutation. Other oncogenic mutations are known in the art and include, but are not limited to, mutations in BRCA1, BRCA2, and p53 or TP53. Preferably the sample is obtained or derived from a subject having a solid tumor or a solid cancer. Preferably the cancer is selected from an adenocarcinoma, more in particular mucinous adenocarcinoma, a pancreatic cancer, more in particular a pancreatic adenocarcinoma, or a renal cell carcinoma, brain cancer, a glioma, a glioblastoma, a pancreatic ductal adenocarcinoma, a sarcoma, a bladder cancer, a colon cancer, a rectal cancer, a colorectal cancer, a gallbladder cancer, a head and neck cancer, a prostate cancer, a uterus cancer, a breast cancer, an ovarian cancer, a liver cancer, an endometrial cancer, a lung cancer, in particular a non-small cell lung cancer or an invasive mucinous adenocarcinoma.

The cancer is preferably an ErbB-2 and/or ErbB-3 positive cancer.

Preferably the sample is obtained from a subject having a primary cancer or primary tumor, a recurrent cancer or recurrent tumor or a metastasized cancer or metastasized tumor.

Preferably the liquid biopsy sample is selected from blood, blood serum, blood plasma, pleural effusion, saliva, urine, semen, sputum, vaginal fluid, amniotic fluid, peritoneal fluid, cerebrospinal fluid, bone marrow, cell free lavage and another biofluid. Methods for obtaining such samples are known in the art and are described in the Examples.

Preferably the volume of the sample is from about 0.1 to about 50 ml, such as from about 0.2 ml to about 11 ml. The volume of the sample is preferably from about 0.5 to about 10 ml. Suitable volumes include, but are not limited to, at least about 0.1 ml, such as at least about 0.5 ml, at least about 1 ml, at least about 5 ml or at least about 10 ml.

The method of the invention is typically conducted on a sample that is known to contain or suspected of containing the NRG1 fusion polynucleotide. Alternatively, the invention may be carried out on any sample whose NRG1 fusion polynucleotide status is unknown to confirm the presence or absence of the NRG1 fusion polynucleotide.

The sample is typically processed prior to being used in the invention, for example by centrifugation or by passage through a membrane that filters out unwanted molecules or cells, such as red blood cells. The sample may be tested immediately upon being taken. The sample may also be typically stored prior to assay, preferably below −70° C. The sample may be processed as described in the Examples before being used in a method of the invention.

### Subjects

Typically, the sample is human in origin, but alternatively it may be from another mammal animal such as from commercially farmed animals such as horses, cattle, sheep, fish, chickens or pigs or may alternatively be pets such as cats or dogs. The sample is preferably human. The sample is preferably obtained from a human subject.

The sample may be obtained/derived from a subject that is known to have or suspected to cancer and/or the NRG1 fusion polynucleotide. Alternatively, the sample may be obtained/derived from a subject whose cancer status and/or NRG1 fusion polynucleotide status is unknown to confirm the presence or absence of cancer and/or the NRG1 fusion polynucleotide. Cancer diagnosis is discussed in more detail below.

### Quantification

The invention also provides a method for measuring the amount or concentration of an NRG1 fusion polynucleotide in a sample. The method comprises conducting any of the methods described above and, if the NRG1 fusion polynucleotide is present, determining its amount or concentration in the sample. Suitable methods for quantifying the amount of NRG1 fusion polynucleotides when using the PCR, sequencing and/or probe detection methods above are well known to a person skilled in the art.

If the NRG1 fusion polynucleotide is present, the method typically comprises evaluating the quantification value and thereby measuring the amount and/or concentration of the NRG1 fusion polynucleotide. The method preferably comprises evaluating the quantification value against standard quantification values generated from standard dilution curves. This is straightforward for PCR based methods.

Preferably, the amount of an NRG1 fusion polynucleotide selected from the NRG1 fusion polynucleotides comprising or consisting of the sequences of SEQ ID NOs: 19, 27, 41, 62, 74, 83, 102, 109, 120, 146, 154, 176, 201, 217, 225, 240, 260, 298, 325, 353, 364, 376, 384, 413, 432, 468 or 469 is detected.

### Multiplex Assays

The invention also provides multiplex assays in which the presence or absence of two or more NRG1 fusion polynucleotides in a sample are determined. Any of the embodiments discussed above equally apply to the multiplex assays of the invention, including isolation, amplification and/or detection using sequencing or probes.

The method may comprise determining the presence or absence of two or more, three or more, four or more, five or more or six or more NRG1 fusion polynucleotides in a sample.

The method may comprise determining the presence or absence of 2, 3, 4, 5 or 6 NRG1 fusion polynucleotides in a sample. Alternatively, the method may comprise determining the presence or absence of 2, 3, 4, 5, 6, 7, 8, 9, 10, 11, 12, 13, 14, 15, 16, 17, 18, 19, 20, 21, 22, 23, 24, 25 or 26 NRG1 fusion polynucleotides in a sample. The NRG1 fusion polynucleotides may be any of those discussed above. The two or more NRG1 fusion polynucleotides are preferably selected from VAPB-NRG1, CADM1-NRG1, CD44-NRG1, SLC3A2-NRG1, VTCN1-NRG1, CDH1-NRG1, CXADR-NRG1, GTF2E2-NRG1, CSMD1-NRG1, PTN-NRG1, ST14-NRG1, THBS1-NRG1, AGRN-NRG1, PVALP-NRG1, APP-NRG1, WRN-NRG1, DAAM1-NRG1, ASPH-NRG1, NOTCH2-NRG1, CD74-NRG1, SDC4-NRG1, SLC4A4-NRG1, ZFAT-NRG1, DSCAML1, RBPMS-NRG1 and ATP1B1-NRG1. These fusion polynucleotides are defined above. The two or more NRG1 fusion polynucleotides are preferably selected from the NRG1 fusion polynucleotides comprising or consisting of the sequences of SEQ ID NOS: 19, 27, 41, 62, 74, 83, 102, 109, 120, 146, 154, 176, 201, 217, 225, 240, 260, 298, 325, 353, 364, 376, 384, 413, 432, 468 or 469.

The multiplex method of the invention may involve amplifying the two or more NRG1 fusion polynucleotides. In such embodiments, the corresponding number of pairs of forward and reverse polynucleotide primers are used as described above. Any of the pairs of polynucleotide primers discussed above may be used.

Sequencing, such as NGS, is the most efficient way of determining the presence or absence of two or more NRG1 fusion polynucleotides. Alternatively, two or more polynucleotide probes which specifically hybridise to the two or more NRG1 fusion polynucleotides may be used. Any of the probes discussed above may be used in the multiplex assay of the invention.

The invention also provides a method for measuring the amount or concentration of two or more NRG1 fusion polynucleotides in a sample. The method comprises conducting any of the multiplex methods described above and, if the two or more NRG1 fusion polynucleotides are present, determining their amount or concentration in the sample. Any embodiments discussed above with reference to the quantification embodiments equally apply here.

### Kits

The invention also provides a kit for determining the presence or absence of an NRG1 polynucleotide fusion in a liquid biopsy sample, comprising (a) a pair of forward and reverse polynucleotide primers which specifically hybridise to the NRG1 fusion polynucleotide and/or (b) a polynucleotide probe which specifically hybridizes to the NRG1 fusion polynucleotide. The invention also provides a kit for determining the presence or absence of two or more NRG1 polynucleotide fusions in a liquid biopsy sample, comprising (a) two or more pairs of forward and reverse polynucleotide primers which specifically hybridise to the two or more NRG1 fusion polynucleotides and/or (b) two or more polynucleotide probes which specifically hybridize to the two or more NRG1 fusion polynucleotide. The kits preferably comprise (a) and (b). In some embodiments, the polynucleotide primers or polynucleotide probe are specific for cDNA. For example, in some embodiments the primers or probes are specific for an exon-exon junction in cDNA.

Any of the primers and probes discussed above may form part of the kits of the invention. The pair(s) of forward and reverse polynucleotide primers preferably specifically hybridise to the NRG1 fusion polynucleotide across the fusion junction. The polynucleotide probe(s) typically specifically hybridise(s) to a target sequence in the NRG1 fusion polynucleotide. The target sequence(s) preferably comprise(s) the fusion junction in the NRG1 fusion polynucleotide, the EGF-like domain of NRG1 in the NRG1 fusion polynucleotide or both the fusion junction and the EGF-like domain of NRG1 in the NRG1 fusion polynucleotide.

The kit preferably further comprises a positive control polynucleotide comprising or consisting of an NRG1 fusion polynucleotide. Any of the NRG1 fusion polynucleotides discussed above may be present in the kit. The kit preferably contains a negative control, such as DNAase-free and RNAase-free water.

The kit may additionally comprise one or more other reagents or instruments which enable any of the embodiments of the methods above to be carried out. Such reagents or instruments include, but are not limited to, one or more of the following: suitable buffer(s) (aqueous solutions), means to obtain a sample from a patient (such as a vessel or an instrument comprising a needle or a swab) or tubes in which quantitative reactions can be done. The kit may, optionally, comprise instructions to enable the kit to be used in the methods of the invention or details regarding which patients may be tested. The kit may further comprise any of the reagents required to conduct PCR discussed above, including a reverse transcriptase and/or heat-stable DNA polymerase.

Preferably, the kit is for detecting an NRG1 fusion polynucleotide selected from the NRG1 fusion polynucleotides comprising or consisting of the sequences of SEQ ID NOs: 19, 27, 41, 62, 74, 83, 102, 109, 120, 146, 154, 176, 201, 217, 225, 240, 260, 298, 325, 353, 364, 376, 384, 413, 432, 468 or 469.

### Diagnostic Methods

The invention also provides a method of determining whether or not a subject has cancer or is prone to developing cancer. In the context of the invention, a subject is prone to developing cancer if they have an increased chance or likelihood of developing cancer. Subjects with one or more NRG1 fusion polynucleotides have an increased chance or likelihood of developing cancer compared with subjects that do not have any NRG1 fusion polynucleotides. The invention therefore relates to the diagnosis or prognosis of cancer. The diagnostic or prognostic method of the invention may be carried out in conjunction with other assays or genetic tests. The method comprises conducting a method of the invention for determining the presence or absence of an NRG1 fusion polynucleotide or two or more NRG1 fusion polynucleotides on a sample from the subject. Any of the methods discussed above may be used. The presence of the NRG1 fusion polynucleotide or two or more NRG1 fusion polynucleotides in the sample typically indicates the subject has cancer or is prone to developing cancer. The absence of the NRG1 fusion polynucleotide or two or more NRG1 fusion polynucleotides from the sample typically indicates the subject does not have cancer and is not prone to developing cancer.

Typically, the subject displays the symptoms of cancer, i.e. the subject is known or expected to have cancer. The subject may be asymptomatic, i.e. the subject's cancer status is unknown or the subject is not expected not to have cancer. The subject may be susceptible to, or at risk from, developing cancer. The subject may be otherwise genetically predisposed to cancer. Other oncogenic mutations are discussed above.

The cancer may be any cancer or tumor discussed above. The cancer is preferably an ErbB-2 and/or ErbB-3 positive cancer.

The subject may be any of those subjects discussed above. The subject is generally a human subject. The subject may be a fetus, a newborn, an infant, a juvenile or an adult.

Preferably, the method of determining whether or not a subject has cancer or is prone to developing cancer comprises a step of detecting an NRG1 fusion polynucleotide selected from the NRG1 fusion polynucleotides comprising or consisting of the sequences of SEQ ID NOs: 19, 27, 41, 62, 74, 83, 102, 109, 120, 146, 154, 176, 201, 217, 225, 240, 260, 298, 325, 353, 364, 376, 384, 413, 432, 468 or 469.

More preferably, the NRG1 polynucleotide portion of any fusion of the present disclosure and identified by the methods and products of the invention disclosed comprises an EGF-like domain. Said EGF-like domain is preferably in-frame with the fusion partner leading to the expression of an oncogenic fusion protein. Hence, the method of the present disclosure allows rapid and straight forward detection of an oncogenic driver in a liquid biopsy sample of a patient and therewith early diagnosis in the development of a tumor or cancer.

### Therapeutic Methods

The invention also provides a method of treating cancer in a subject identified as being having cancer using a method of the invention. The method comprises administering to the subject a therapeutically effective amount of an anti-cancer treatment. Preferably, the therapeutic method is for treating a subject that comprises an NRG1 fusion polynucleotide selected from the NRG1 fusion polynucleotides comprising or consisting of the sequences of SEQ ID NOs: 19, 27, 41, 62, 74, 83, 102, 109, 120, 146, 154, 176, 201, 217, 225, 240, 260, 298, 325, 353, 364, 376, 384, 413, 432, 468 or 469 wherein the subject has cancer or is prone to developing cancer.

The invention also provides a method of treating cancer in a subject. The method comprises (a) identifying the subject as having cancer using a method of the invention and (b) administering to the subject a therapeutically effective amount of an anti-cancer treatment.

The invention also provides a substance or composition for use in a method of treating cancer in a subject identified as having using a method of the invention. The invention also provides a substance or composition for use in a method of treating cancer in a subject, wherein the method comprises (a) identifying the subject as having cancer using a method of the invention and (b) administering the substance of composition to the subject. The substance or composition is preferably an anti-cancer substance or composition.

The cancer may be any cancer or tumor discussed above. The cancer is preferably an ErbB-2 and/or ErbB-3 positive cancer.

Any treatment, substance or composition may be used in the invention. Suitable anti-cancer treatments, substances and compositions are well known. The anti-cancer treatment is preferably one or more of surgery, chemotherapy, radiotherapy, hormone therapy, stem cell and bone marrow transplant, a targeting cancer drug, immunotherapy, a bisphosphonate, gene therapy, photodynamic therapy and cryotherapy.

Anti-cancer immunotherapy includes monoclonal antibodies, checkpoint inhibitors, cytokines, vaccines and CAR-T cells. Examples of monoclonal antibodies are rituximab (Mabthera), a treatment for chronic lymphocytic leukaemia (CLL) and some types of non Hodgkin lymphoma, cetuximab (Erbitux), a treatment for advanced bowel cancer and head and neck cancer, and trastuzumab (Herceptin), used to treat breast cancer and stomach cancer. Checkpoint inhibitors typically target CTLA-4 (cytotoxic T lymphocyte associated protein 4), PD-1 (programmed cell death protein 1), PD-L1 (programmed cell death ligand 1) or CTLA-4 and PD-1 are found on T cells. Checkpoint inhibitors that block PD-1 include nivolumab (Opdivo) and pembrolizumab (Keytruda). Ipilimumab (Yervoy) is a checkpoint inhibitor drug that blocks CTLA-4. Checkpoint inhibitors that block PD-L1 include atezolizumab, avelumab and durvalumab. Suitable cytokines are interferon alpha and aldesleukin. Examples of cancer vaccines include antigen vaccines, whole cell vaccines, dendritic cell vaccine, DNA vaccines and anti-idiotypic vaccines.

As discussed above, the receptors for NRG1 isoforms are the ErbB family of tyrosine kinase transmembrane receptors. The substance, composition or treatment is preferably an ErbB-2 and/or ErbB-3 targeting agent.

The term ‘ErbB-2’ as used herein refers to the protein that in humans is encoded by the ERBB-2 gene. Alternative names for the gene or protein include CD340; HER-2; HER-2/neu; MLN 19; NEU; NGL; TKR1. The ERBB-2 gene is frequently called HER2 (from human epidermal growth factor receptor 2). Where reference is made herein to ErbB-2, the reference refers to human ErbB-2. An antibody comprising an antigen-binding site that binds ErbB-2, binds human ErbB-2. The ErbB-2 antigen-binding site may, due to sequence and tertiary structure similarity between human and other mammalian orthologs, also bind such an ortholog but not necessarily so. Database accession numbers for the human ErbB-2 protein and the gene encoding it are (NP_001005862.1; NP_004439.2; NC_000017.10; NT_010783.15; NC_018928.2). The accession numbers are primarily given to provide a further method of identification of ErbB-2 as a target, the actual sequence of the ErbB-2 protein bound the antibody may vary, for instance because of a mutation in the encoding gene such as those occurring in some cancers or the like. The ErbB-2 antigen binding site binds ErbB-2 and a variety of variants thereof, such as those expressed by some ErbB-2 positive tumor cells. The antigen-binding site that binds ErbB-2 preferably binds domain I of ErbB-2.

The term ‘ErbB-3’ as used herein refers to the protein that in humans is encoded by the ERBB3 gene. Alternative names for the gene or protein are HER3; LCCS2; MDA-BF-1; c-ErbB-3; c-ErbB3; ErbB3-S; p180-ErbB3; p45-sErbB3; and p85-sErbB3. Where reference is made herein to ErbB-3, the reference refers to human ErbB-3. An antibody comprising an antigen-binding site that binds ErbB-3, binds human ErbB-3. The ErbB-3 antigen-binding site may, due to sequence and tertiary structure similarity between human and other mammalian orthologs, also bind such an ortholog but not necessarily so. Database accession numbers for the human ErbB-3 protein and the gene encoding it are (NP_001005915.1, NP_001973.2, NC_000012.11, NC_018923.2, NT_029419.12). The accession numbers are primarily given to provide a further method of identification of ErbB-3 as a target, the actual sequence of the ErbB-3 protein bound by an antibody may vary, for instance because of a mutation in the encoding gene such as those occurring in some cancers or the like. The ErbB-3 antigen binding site binds ErbB-3 and a variety of variants thereof, such as those expressed by some ErbB-3 positive tumor cells. The antigen-binding site that binds ErbB-3 preferably binds domain III of ErbB-3.

When reference is made to ErbB-2 or ErbB-3 or an alternative name for the same, the reference is to human ErbB-2 or ErbB-3. Antibodies as referred to herein bind to ErbB-2 or ErbB-3 and many mutated ErbB-2 or ErbB-3 proteins as can be found in cancers.

The ErbB-2 and/or ErbB-3 targeting agent is preferably selected from the group consisting of a multispecific antibody comprising a first antigen-binding site that binds an extracellular part of ErbB-2 and a second antigen-binding site that binds an extracellular part of ErbB-3, a tyrosine kinase inhibitor of ErbB-2, a monospecific bivalent antibody comprising an antigen-binding site that bind an extracellular part of ErbB-2, a monospecific bivalent antibody comprising an antigen-binding site that bind an extracellular part of ErbB-3, and any combination thereof.

Preferably, said targeting agent is for treatment of a cancer that expresses an NRG1 fusion polypeptide, wherein the cancer is preferably an ErbB-2 and/or ErbB-3 positive cancer.

Also included for the treatment cancer is the administration of one or more compounds selected from the group consisting of an inhibitor of a component of the PI3Kinase pathway, an inhibitor of a component of the MAPK pathway, a microtubuli disrupting drug, and an inhibitor of a histone deacetylase (HDAC). The inhibitor preferably comprises a tyrosine kinase inhibitor, a PI3Ka inhibitor, an Akt inhibitor, an mTOR inhibitor or an Src inhibitor. The tyrosine kinase inhibitor is preferably afatinib, lapatinib and/or neratinib. The PI3Ka inhibitor is preferably BYL719. In an embodiment, the Akt inhibitor is MK-2206. In a disclosed embodiment, the mTOR inhibitor is everolimus. In a disclosed embodiment, the Src inhibitor is saracatinib. In a disclosed embodiment, the microtubuli disrupting drug is paclitaxel. In a disclosed embodiment, the HDAC inhibitor is vorinostat. In an embodiment, the binding compound that is specific for ErbB 2 and ErbB 3 is MM 111.

The ErbB-2 targeting agent is preferably with a monospecific bivalent antibody comprising antigen-binding sites that bind an extracellular part of ErbB-2. Such antibody is preferably trastuzumab, pertuzumab, or trastuzumab-emtansine. The ErbB-2 targeted treatment is preferably an ErbB-2 TKI. The ErbB-2 TKI is preferably one or more of lapatinib, canertinib, neratinib, tucatinib (or irbinitinib), CP-724714, tarloxitinib, mubritinib, afatinib, varlitinib, and dacomitinib, preferably afatinib. An ErbB-2 TKI may also affect ErbB-1 signaling but is different from an ErbB-1 TKI in that it has significant activity on ErbB-2. The ErbB-3 targeted treatment is preferably with a monospecific bivalent antibody comprising antigen-binding sites that bind an extracellular part of ErbB-3. Such antibody is preferably patritumab, seribantumab, lumretuzumab, elgemtumab, GSK2849330, KTN3379 or AV-203.

Preferably, the monospecific bivalent antibody comprising antigen-binding sites that bind an extracellular part of ErbB-3 comprises patritumab, seribantumab, lumretuzumab, elgemtumab, GSK2849330, KTN3379 or AV-203.

The tyrosine kinase inhibitor is preferably afatinib, lapatinib and/or neratinib.

The antibody can preferably reduce a ligand-induced receptor function of ErbB-3 on a ErbB-2 and ErbB-3 positive cell. Also, the antibody can preferably reduce ligand-induced growth of an ErbB-2 and ErbB-3 positive cell. The antibody is preferably a multispecific or bispecific antibody comprising a first antigen-binding site that binds an extracellular part of ErbB-2, in particular domain I thereof, and a second antigen-binding site that binds an extracellular part of ErbB-3, in particular domain III thereof. Most preferably, the bispecific antibody comprises or consists of Zenocutuzumab (CAS Registry Number 1969309-56-5; International Nonproprietary Names for Pharmaceutical Substances (INN), WHO Drug Information, Vol. 31, No. 4, 2017 Proposed INN: List 118). In a clinical trial referred to as the eNRGy study and the Early Access Program (clinical trials.gov identifier NCT02912949 and NCT04100694, respectively), Zenocutuzumab, or MCLA-128, is used as the therapeutic targeting agent to treat cancers that comprise an NRG1-fusion, with investigator-assessed responses observed according to RECIST 1.1 criteria across fusion partners and in multiple different cancer types. Investigator-assessed responses were observed according each category in Table 1.

### Prevalence of NRG1 Fusion Partners

TABLE 1
overview of the percentage of NRG1 fusion partners found expressed as
percentage of the indicated fusion partner from the total number of
fusions identified in individuals in clinical trial NCT02912949 with cut-off
of Apr. 12^{th}, 2022. Other comprises NRG1 fusions identified at less
than 2% of the total identified as of the cut-off date.

| NRG1 fusion partner category | Percentage of total |
|---|---|
| CD74 | 31% |
| SLC3A2 | 16% |
| SDC4 | 9% |
| RBPMS | 4% |
| CDH1 | 2% |
| VTCN1 | 2% |
| Others | 23% |

Thus, in a disclosed embodiment, the present disclosure includes Zenocutuzumab for use in the treatment of cancer in a subject wherein the presence or absence of an NRG1 fusion polynucleotide in a sample from said subject is determined by 1) providing a liquid biopsy sample, and 2) determining the presence or absence of the NRG1 fusion polynucleotide in the sample, wherein the NRG fusion polynucleotide is a fusion of an NRG1 polynucleotide with a second polynucleotide, which second polynucleotide does not encode NRG1, wherein if the sample is determined to comprise the presence of the NRG1 fusion polynucleotide, said subject is administered a therapeutically effective amount of Zenocutuzumab thereby treating the cancer in the subject.

The invention concerns administering to the subject a therapeutically effective amount of the treatment, substance or composition to the subject. A therapeutically effective amount is an amount which ameliorates one or more symptoms of the cancer. A therapeutically effective amount is preferably an amount which abolishes one or symptoms of the cancer. A therapeutically effective amount may cure or abolish the cancer. A therapeutically effective amount is preferably an amount which prevents progression or spread of the cancer. A therapeutically effective amount may cure or abolish the cancer. Suitable amounts are discussed in more detail below.

The treatment, substance or composition of the invention may be administered to any suitable subject. Suitable subjects are discussed above.

The invention may be used in combination with other means of, and substances for, treating the disease or disorder or providing pain relief. In some cases, the treatment, substance or composition may be used in combination with existing treatments for cancer including intensive care treatment and the use of ventilators.

The treatment, substance or composition of the invention may be formulated using any suitable method. Formulation with standard pharmaceutically acceptable carriers and/or excipients may be carried out using routine methods in the pharmaceutical art. The exact nature of a formulation will depend upon several factors including the composition to be administered and the desired route of administration. Suitable types of formulation are fully described in Remington's Pharmaceutical Sciences, 19^{th} Edition, Mack Publishing Company, Eastern Pennsylvania, USA.

The treatment, substance or composition may be administered by any route. Suitable routes include, but are not limited to, enteral or parenteral routes such as via buccal, anal, pulmonary, intravenous, intra-arterial, intramuscular, intraperitoneal, intraarticular, topical and other appropriate routes. If the lungs are being treated, the treatment, substance or composition is preferably administered by inhalation.

Pharmaceutical compositions may be prepared together with a physiologically acceptable carrier or diluent. The treatment, substance or composition may be mixed with an excipient which is pharmaceutically acceptable and compatible with the active ingredient. Suitable excipients are, for example, water, saline, dextrose, glycerol, of the like and combinations thereof.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active substance, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

For suppositories, traditional binders and carriers may include, for example, polyalkylene glycols or triglycerides; such suppositories may be formed from mixtures containing the active ingredient in the range of 0.5% to 10%, preferably 1% to 2%.

Oral formulations include such normally employed excipients as, for example, pharmaceutical grades of mannitol, lactose, starch, magnesium stearate, sodium saccharine, cellulose, magnesium carbonate, and the like. These compositions typically take the form of solutions or suspensions and contain 10% to 95% of active ingredient, preferably 25% to 70%. Where the pharmaceutical composition is lyophilised, the lyophilised material may be reconstituted prior to administration, e.g. a suspension. Reconstitution is preferably effected in buffer.

In addition, if desired, the pharmaceutical compositions of the invention may contain minor amounts of auxiliary substances such as wetting or emulsifying agents, pH buffering agents, and/or adjuvants which enhance effectiveness. The composition preferably comprises human serum albumin.

One suitable carrier or diluents is Plasma-Lyte AR. This is a sterile, nonpyrogenic isotonic solution for intravenous administration. Each 100 mL contains 526 mg of Sodium Chloride, USP (NaCl); 502 mg of Sodium Gluconate (C6H11NaO7); 368 mg of Sodium Acetate Trihydrate, USP (C2H3NaO2·3H2O); 37 mg of Potassium Chloride, USP (KCl); and 30 mg of Magnesium Chloride, USP (MgCl2·6H2O). It contains no antimicrobial agents. The pH is adjusted with sodium hydroxide. The pH is 7.4 (6.5 to 8.0).

If the treatment, substance or composition is a siRNA, the most disclosed pharmaceutically acceptable carrier or diluent is a pharmaceutically acceptable transfection reagent. A pharmaceutical acceptable transfection reagent is suitable for administration to subjects. The pharmaceutical acceptable transfection reagent may be liposomes, preferably cationic liposomes, polymers, preferably cationic polymers, and dendrimers. The pharmaceutical acceptable transfection reagent is more preferably a pharmaceutically acceptable PEI transfection reagent, such as a linear PEI transfection reagent. A disclosed pharmaceutical acceptable transfection reagent is GMP in vivo-jetPEI®.

The composition is administered in a manner compatible with the dosage formulation and in such amount will be therapeutically effective. The quantity to be administered depends on the subject to be treated, capacity of the subject's immune system and the degree of treatment desired. Precise amounts required to be administered may depend on the judgement of the practitioner and may be peculiar to each subject.

Any suitable dose of the treatment, substance or composition may be administered to a subject. The dose may be determined according to various parameters, especially according to the substance used; the age, weight and condition of the subject to be treated; the route of administration; and the required regimen. Again, a physician will be able to determine the required route of administration and dosage for any particular subject. A typical daily dose is from about 0.01 to 50 mg per kg of body weight, according to the activity of the specific substance, the age, weight and conditions of the subject to be treated and the frequency and route of administration. Preferably, daily dosage levels are from 5 mg to 2 g. For example, from about 0.01 to about 50 mg per kg of subject of sRNA may administered, such as from about 0.05 to about 40, from about 0.1 to about 30, from about 0.5 to about 20, from about 1 to about 10 or from about 2 to about 5 mg per kg. At least about 0.01 mg per kg of subject may administered, such as at least about 0.05, at least about 0.1, at least about 0.5, at least about 1, at least about 2, at least about 5, at least about 10, at least about 20, at least at least about 30 or at least about 40 mg per kg.

These doses may be provided as a single dose or may be provided as multiple doses, for example taken at regular intervals, for example 2, 3 or 4 doses administered daily. Other suitable regular intervals include, but are not limited to, every day, every week, every fortnight or every month.

As discussed above, a disclosed anti-cancer treatment is a bispecific antibody which inhibits ErbB-2 and ErbB3. The amount of bispecific antibody to be administered to a subject is typically in the therapeutic window, meaning that a sufficient quantity is used for obtaining a therapeutic effect, while the amount does not exceed a threshold value leading to an unacceptable extent of side-effects. The lower the amount of antibody needed for obtaining a desired therapeutic effect, the larger the therapeutic window will typically be. The selected dosage level will depend upon a variety of factors including the route of administration, the time of administration, the rate of excretion of the particular compound being employed, the duration of the treatment, other drugs, compounds and/or materials used in combination, the age, sex, weight, condition, general health and prior medical history of the subject being treated, and like factors well known in the medical arts. The dosage can be in the range of the dosing regimen for trastuzumab or lower.

With respect to the bispecific antibody of the present disclosure, in particular Zenocutuzumab, it has a good safety profile at relatively high doses, thus providing a large therapeutic window. Dosing of the bispecific antibody of the present invention follows a weekly, biweekly or tri-weekly administration regimen of 750 mg, preferably a bi-weekly dose of 750 mg. The dosing is preferably in subjects with pancreatic cancer, NSCLC or a solid tumor, and includes any subject having a solid tumor harboring an NRG1 fusion polynucleotide, wherein such subject has progressed upon the administration of chemotherapy or an ErbB-2 or ErbB-3 targeting agent or TKI. Alternatively, a dosing regimen is followed comprising a weekly flat dose administration of 400 mg, preferably commenced after a single administration of 800 mg. Following this alternative dosing regimen, the bispecific antibody of the invention is preferably administered in a weekly dose of 400 mg for 3 weeks followed by 1 week without dosing. Next, one or more cycles of a period of four weeks, consisting of three weekly flat dosages of 400 mg, followed by a week without administration is followed. This is preferably followed until a therapeutic effect is observed.

Dosing preferably involves intravenous injections of two infusions of the bispecific antibody of the invention to arrive at the complete dose, preferably when dosing >360 mg antibody. Alternatively, a single infusion of the complete dose may be given for lower dosages, for instance when dosing ≤360 mg antibody. Pre-medication maybe included in the dosing regimen to mitigate infusion-related reactions.

### EXAMPLES

### Example 1: Test Sample Preparation

Liquid samples or biopsies include blood, blood serum, blood plasma, pleural effusion, urine, semen, vaginal fluid, amniotic fluid, peritoneal fluid, cell free lavage or other biofluids.

When blood is used as starting material, the blood can be collected in tubes treated with cell stabilizing agents such as Cell-Free DNA BCT tubes (Streck) which prevents lysis of blood cells post-collection and minimized contamination of wild-type DNA/RNA from normal cells.

### Example 2 Purification of Circulating DNA from Plasma

Although the protocol of this example is described in relation to the isolation of cfDNA from a plasma sample of 1 ml, the protocol also suitably allows for isolation from other volumes when higher yields are desired and other sample types, like urine and serum. The protocol is based on the QIAamp® circulating Nucleic Acid Kit (CNA) (Qiagen, Cat #55114) and Handbook dated 10/2019 (HB-0202-006).

Before starting, plasma samples from a human subject are allowed to equilibrate to room temperature, either from storage at a suitable refrigerated temperature or directly after drawing from a patient. All centrifugation steps are carried out at room temperature. Where necessary, samples with a volume of below 1 ml are adjusted with phosphate buffered saline (PBS) to a total of 1 ml.

### Preparation of Buffers and Reagents.

Buffers ACB, ACW1 and ACW2 are prepared according to the manufacturer's instructions. Briefly, before use, 200 ml isopropanol (100 v/v %) is added to 300 ml buffer ACB concentrate to obtain 500 ml Buffer ACB. After adding isopropanol, the obtained buffer is mixed well. Before use, 25 ml ethanol 96-100 (v/v %) is added to 19 ml buffer ACW1 concentrate to obtain 44 ml buffer ACW1. The obtained liquid is mixed well after adding ethanol. Before use, 30 ml ethanol (96-100%) is added to 13 ml buffer ACW2 concentrate to obtain 43 ml buffer, which is subsequently well mixed. For instance, to process 12 samples of 1 ml plasma, 10.6 ml buffer ACL is mixed with 67.5 μl carrier RNA-containing AVE buffer.

Isolation of cfDNA from Plasma

Procedure: 100 μl QIAGEN Proteinase K is pipetted into a 50 ml centrifuge tube, 1 ml of plasma is added to the 50 ml tube. 0.8 ml ACL buffer (containing 1.0 μg carrier RNA) is added after which caps are closed and mixed by pulse-vortexing for 30 s while a visible vortex is formed in the tube. Sample and ACL buffer are mixed thoroughly to yield a homogeneous solution. The samples are immediately lysed by incubating at 60° C. for 30 minutes. 1.8 ml ACB buffer is added to the lysate in the tube after which the cap is closed and mix thoroughly by pulse-vortexing for 15-30 s. The lysate-ACB buffer mixture is incubated in the tube for 5 min on ice. QIAamp Mini columns are inserted into a VacConnector on a QIAvac 24 Plus which is set-up according the manufacturer's instructions. A 20 ml tube extender is firmly inserted into the open QIAamp Mini column.

After incubation on ice, the lysate-ACB buffer mixture is applied into the tube extender of the QIAamp Mini column. The vacuum pump is switched on and when all lysates are drawn through the columns completely, the vacuum pump is switched off and pressure is released to 0 mbar. The tube extender is carefully removed and discarded. If part of the QIAvac Connecting System, a Vacuum Regulator can be used. Cross-contamination is avoided by not moving the tube extenders over neighboring QIAamp Mini Columns. 600 μl ACW1 buffer is applied to the QIAamp Mini column. Lids of the column are left open and the vacuum pump switched on. After all of the ACW1 buffer has been drawn through the QIAamp Mini column, the vacuum pump is switched off and pressure is released to 0 mbar. 750 μl ACW2 buffer is applied to the QIAamp Mini column. The lid of the column is left open, and the vacuum pump switched on. After all of ACW2 buffer is drawn through the QIAamp Mini column, the vacuum pump is switched off and pressure is released to 0 mbar. 750 μl of ethanol (96 v/v %) is applied to the QIAamp Mini column. The lid of the column is left open, and the vacuum pump switched on. After all of ethanol is drawn through the spin column, the vacuum pump is switched off and pressure is released to 0 mbar. The lid of the QIAamp Mini column is closed and the column is removed from the vacuum manifold after which the VacConnector is discarded. The QIAamp Mini column is placed in a clean 2 ml collection tube, and centrifuged at full speed (20,000×g; 14,000 rpm) for 3 min. The QIAamp Mini Column is placed in a new 2 ml collection tube. The lid is opened and the assembly is incubated at 56° C. for 10 min to completely dry the membrane. The QIAamp Mini column is placed in a clean 1.5 ml elution tube and the lastly used 2 ml collection tube is discarded. 20 μl of AVE buffer is carefully applied to the center of the QIAamp Mini membrane. The lid is closed and incubated at room temperature for 3 min. Elution buffer AVE is equilibrated to room temperature. The recovered eluate volume is about 5 μl less than the elution volume applied to the QIAamp Mini column. The columns are centrifuged in a microcentrifuge at full speed (20,000×g; 14,000 rpm) for 1 min to elute the nucleic acids such that cell free DNA is obtained.

Free-circulating cell-free DNA is eluted in AVE buffer, ready for use in amplification reactions or storage at −30 to −15° C. Purified nucleic acids are free of proteins, nucleases, and other impurities.

Next, the concentration of the nucleic acids is determined prior to NRG1 fusion amplification and detection analysis according to the manufacturer's recommendation, such as quantitative amplification assays.

List of materials for the QIAvac 24 Plus System for vacuum processing of 1-24 QIAGEN spin columns. QIAvac 24 Plus: Vacuum Manifold for processing 1-24 spin columns which includes QIAvac 24 Plus Vacuum Manifold, Luer Plugs and Quick Couplings (Cat. No. 19413). VacConnectors (500): 500 disposable connectors for use with QIAamp Mini columns on luer slots or VacValves (Cat. No. 19407). VacValves (24): 24 valves for use with the QIAvac 24 Plus (Cat. No. 19408). Vacuum Regulator: For use with QIAvac manifolds (Cat. No. 19530). Vacuum Pump (230 V, 50 Hz): Universal vacuum pump (capacity 34 L/min, 8 mbar vacuum abs.) (Cat. No. 84020). QIAvac Connecting System: System to connect vacuum manifold with vacuum pump: includes tray, waste bottles, tubing, couplings, valve, gauge, 24 VacValves (Cat. No. 19419).

### Example 3: Circulating Tumor RNA (ctRNA)

Purification of Circulating RNA from Serum

Although the protocol of this example is described in relation to the isolation of circulating RNA from a serum sample of 4 ml, the protocol also suitably allows for isolation from other volumes when higher yields are desired and other sample types, like urine or plasma. The protocol is based on the QIAamp® circulating Nucleic Acid Kit (CNA) (Qiagen, Cat #55114) and Handbook dated 10/2019 (HB-0202-006).

Before starting, serum samples from a human subject are allowed to equilibrate to room temperature, either from storage at a suitable refrigerated temperature or directly after drawing from a patient. All centrifugation steps are carried out at room temperature. Where necessary, samples with a volume of below 4 ml are adjusted with phosphate buffered saline (PBS) to a total of 4 ml.

### Preparation of Buffers and Reagents.

Buffers ACB, ACW1 and ACW2 are prepared according to the manufacturer's instructions. Briefly, before use, 200 ml isopropanol (100 v/v %) is added to 300 ml buffer ACB concentrate to obtain 500 ml Buffer ACB. After adding isopropanol, the obtained buffer is mixed well. Before use, 25 ml ethanol 96-100 (v/v %) is added to 19 ml buffer ACW1 concentrate to obtain 44 ml buffer ACW1. The obtained liquid is mixed well after adding ethanol. Before use, 30 ml ethanol (96-100%) is added to 13 ml buffer ACW2 concentrate to obtain 43 ml buffer, which is subsequently well mixed. For instance, to process 12 samples of 4 ml serum, 42.2 ml buffer ACL is mixed with 67.5 μl carrier RNA-containing AVE buffer.

Isolation of cfRNA from Serum

Procedure: 400 μl QIAGEN Proteinase K is pipetted into a 50 ml centrifuge tube, 4 ml of serum is added to the 50 ml tube. 3.2 ml ACL buffer (containing 1.0 μg carrier RNA) is added after which caps are closed and mixed by pulse-vortexing for 30 s while a visible vortex is formed in the tube. Sample and ACL buffer are mixed thoroughly to yield a homogeneous solution. The samples are immediately lysed by incubating at 60° C. for 30 minutes. 7.2 ml ACB buffer is added to the lysate in the tube after which the cap is closed and mix thoroughly by pulse-vortexing for 15-30 s. The lysate-ACB buffer mixture is incubated in the tube for 5 min on ice. QIAamp Mini columns are inserted into a VacConnector on a QIAvac 24 Plus which is set-up according the manufacturer's instructions. A 20 ml tube extender is firmly inserted into the open QIAamp Mini column.

After incubation on ice, the lysate-ACB buffer mixture is applied into the tube extender of the QIAamp Mini column. The vacuum pump is switched on and when all lysates are drawn through the columns completely, the vacuum pump is switched off and pressure is released to 0 mbar. The tube extender is carefully removed and discarded. If part of the QIAvac Connecting System, a Vacuum Regulator can be used. Cross-contamination is avoided by not moving the tube extenders over neighboring QIAamp Mini Columns. 600 μl ACW1 buffer is applied to the QIAamp Mini column. Lids of the column are left open and the vacuum pump switched on. After all of the ACW1 buffer has been drawn through the QIAamp Mini column, the vacuum pump is switched off and pressure is released to 0 mbar. 750 μl ACW2 buffer is applied to the QIAamp Mini column. The lid of the column is left open, and the vacuum pump switched on. After all of ACW2 buffer is drawn through the QIAamp Mini column, the vacuum pump is switched off and pressure is released to 0 mbar. 750 μl of ethanol (96 v/v %) is applied to the QIAamp Mini column. The lid of the column is left open, and the vacuum pump switched on. After all of ethanol is drawn through the spin column, the vacuum pump is switched off and pressure is released to 0 mbar. The lid of the QIAamp Mini column is closed and the column is removed from the vacuum manifold after which the VacConnector is discarded. The QIAamp Mini column is placed in a clean 2 ml collection tube, and centrifuged at full speed (20,000×g; 14,000 rpm) for 3 min. The QIAamp Mini Column is placed in a new 2 ml collection tube. The lid is opened and the assembly is incubated at 56° C. for 10 min to completely dry the membrane. The QIAamp Mini column is placed in a clean 1.5 ml elution tube and the lastly used 2 ml collection tube is discarded. 20 μl of AVE buffer is carefully applied to the center of the QIAamp Mini membrane. The lid is closed and incubated at room temperature for 3 min. Elution buffer AVE is equilibrated to room temperature. The recovered eluate volume is about 5 μl less than the elution volume applied to the QIAamp Mini column. The columns are centrifuged in a microcentrifuge at full speed (20,000×g; 14,000 rpm) for 1 min to elute the nucleic acids such that cell free RNA is obtained.

Free-circulating cell-free RNA is eluted in AVE buffer, ready for use in amplification reactions or storage at −30 to −15° C. Purified nucleic acids are free of proteins, nucleases, and other impurities.

Next, the concentration of the nucleic acids is determined prior to NRG1 fusion amplification and detection analysis according to the manufacturer's recommendation, such as quantitative amplification assays.

List of materials for the QIAvac 24 Plus System for vacuum processing of 1-24 QIAGEN spin columns. QIAvac 24 Plus: Vacuum Manifold for processing 1-24 spin columns which includes QIAvac 24 Plus Vacuum Manifold, Luer Plugs and Quick Couplings (Cat. No. 19413). VacConnectors (500): 500 disposable connectors for use with QIAamp Mini columns on luer slots or VacValves (Cat. No. 19407). VacValves (24): 24 valves for use with the QIAvac 24 Plus (Cat. No. 19408). Vacuum Regulator: For use with QIAvac manifolds (Cat. No. 19530). Vacuum Pump (230 V, 50 Hz): Universal vacuum pump (capacity 34 L/min, 8 mbar vacuum abs.) (Cat. No. 84020). QIAvac Connecting System: System to connect vacuum manifold with vacuum pump: includes tray, waste bottles, tubing, couplings, valve, gauge, 24 VacValves (Cat. No. 19419).

To avoid loss of RNA from biological material during and after purification, any plasticware or glassware is first thoroughly cleaned to eliminate possible RNase contamination. Inadvertent introduction of RNases into the isolation procedure is avoided by creating and maintaining an RNase-free environment.

### Example 4: Isolation of Circulating Tumor Cells

1. This protocol describes isolation of circulating tumor cells from whole blood using the AdnaTest BreastCancerSelect kit (ADNAGEN, catalog #T-1-508), which enables immunomagnetic enrichment of tumor cells via epithelial and tumor-associated antigens. Antibodies against epithelial and tumor-associated antigens are conjugated to magnetic beads (Dynabeads) for labeling tumor cells in peripheral blood. Labeled cells are extracted by a magnetic particle concentrator (AdnaMag-L and AdnaMag-S) and are subsequently lysed. mRNA is isolated from the resulting lysate and used for NRG1 fusion detection.
2. Whole blood (at least 5 ml, but 10 ml is also possible) is collected in tubes containing EDTA ((e.g. ‘S Monovette® Kalium EDTA’, Sarstedt; ‘BD Vacutainer® K3EDTA’, Becton Dickinson) and is immediately placed on ice following the draw and processed within 4 hours.
3. Preparation of Select Beads: The BreastSelect Beads (provided in the kit) are resuspended thoroughly by pipetting. The volume of BreastSelect Beads required for all samples is calculated and the calculated volume transferred to 1.5 ml reaction tubes. The tubes are placed in AdnaMag-S Magnetic particle concentrator (AdnaGen GmbH, cat. No. T-1-800). Supernatant is removed after 1 min. Beads are washed with 1 ml PBS (Phosphate buffer saline; pH 7.0-7.3) three times. The tubes are removed from the AdnaMag-S and resuspended in 100 μl PBS.
4. Selection of Tumor cells: 5 ml of blood sample was first pipetted into a 15 ml tube. 100 μl of resuspended BreastSelect Beads are added to each blood sample. The tubes are rotated slowly (approx. 5 rpm) for 30 min at room temperature on a device allowing both tilting and rotation. The tubes are then placed into the AdnaMag-L (AdnaGen GmbH, cat. No. T-1-700) without magnetic slider, which is swung downwards to release blood drops captured in the cap. The magnetic slider is then inserted and the tubes incubated for 3 min at room temperature. The blood supernatant is removed completely with a 10 ml pipet without touching the beads. Washing is done three times by first removing the magnetic slider from the AdnaMag-L, adding 5 ml of PBS to the tubes and gentle resuspension of the magnetic bead/cell complex gently. The magnetic slider is placed back into the AdnaMag-L and tubes are incubated for 1 min at room temperature, followed by removal of the supernatant with a pipet. After washing, the magnetic slider is removed and bead/cell complexes are resuspended in 1 ml PBS and transferred to a 1.5 ml reaction tube. The reaction tube is placed into the AdnaMag-S with an inserted magnetic slider. After 1 min, the supernatant is completely removed for the subsequent cell lysis.
5. The magnetic slider is removed from the AdnaMag-S and 200 μl of room temperature equilibrated Lysis/Binding buffer (provided in the kit) is added to each reaction tube followed by resuspension by pipetting five times. The magnetic slider is inserted into the AdnaMag-S and incubated for 1 min. The supernatants containing the cell lysates are transferred to a new 1.5 ml reaction tubes and the tubes containing the beads are discarded. The cell lysates are suitable for mRNA-isolation or stored at −20° C. mRNA of the lysate is isolated using Oligo (dT) primers in a reverse transcription (RT-PCR) reaction and transcribed into cDNA template. NRG1 fusions are subsequently detected by PCR mediated amplification.

### Example 5: Extracellular Vesicles and Exosomes

Total vesicular RNA is obtained from blood using the ExoRNeasy Maxi kit (cat. No. 77023, QIAGEN GmbH, Hilden, Germany) which is based on membrane-based affinity binding step to isolate exosomes and other EVs from cell-free biofluids.

Step 1 includes plasma separation and storage. Whole blood is collected in BD Vacutainer® Venous Blood Collection Tubes (cat. no. 367525) containing EDTA (or PAXgene® Blood ccfDNA Tubes [cat. no. 768115] and Cell-Free DNA BCT®—but not RNA BCT-tubes from Streck). The tubes are stored at room temperature or 4° C. and processed within 1 hour. Blood samples are centrifuged in primary blood collection tubes for 10 min at 1900×g (3000 rpm) and 4° C. using a swinging bucket rotor. The upper (yellow) plasma phase is carefully transferred to a new tube (with conical bottom) without disturbing the intermediate buffy coat layer (containing white blood cells and platelets). Plasma samples are centrifuged in conical tubes for 15 min at 3000×g (or 10 min at 16,000×g-see above) and 4° C. or passed through a 0.8 μm filter (Sartorius® Minisart® NML [Sartorius cat. no. 16592]). The cleared supernatant is carefully transferred to a new tube without disturbing the pellet. The plasma is stored at 2-8° C. for up to 6 h and processed on the same day.

Step 2 describes capture and lysis of exosomes and other extracellular vesicles (EVs) from 4 ml of plasma. 4 ml of binding buffer XBP is added to 4 ml of plasma sample and mixed well immediately by gently inverting the tube 5 times. The plasma/XBP mix is added onto the exoEasy spin column (membrane affinity column which binds the EVs to the membrane) and centrifuged for 1 min at 500×g. The flow-through is discarded and the column is placed back into the same collection tube. 10 ml wash buffer XWP is added to the exoEasy Maxi spin column, and centrifuged for 5 min at 5000×g. The flow-through is discarded together with the collection tube. The spin column is transferred to a fresh collection tube. 700 μl QIAzol is added to the membrane for lysis of vesicles, centrifuged for 5 min at 5000×g to collect the lysate, and transferred completely to a 2 ml tube.

Step 3 describes isolation of total RNA from the lysate. Buffers RWT and RPE are prepared according to the manufacturer's instructions. The tube containing the lysate is briefly vortexed and incubated at room temperature for 5 min. 90 μl chloroform is then added to the tube and shaken vigorously for 15 s. After incubation at room temperature for 2-3 min, the tube is centrifuged for 15 min at 12,000×g at 4° C. The upper aqueous phase is transferred to a new collection tube, 2 volumes of 100% ethanol added and mixed thoroughly. 700 μl sample is pipetted into an RNeasy MinElute spin column in a 2 ml collection tube and centrifuged at ≥8000×g (≥10,000 rpm) for 15 s at room temperature. After discarding the flow-through, this step is repeated using the remainder of the sample. 700 μl Buffer RWT is added to the RNeasy MinElute spin column and centrifuged for 15 s at ≥8000×g (≥10,000 rpm), followed by 500 μl Buffer RPE and centrifuged for 15 s at ≥8000×g (≥10,000 rpm). Flow-through is discarded at each step. Finally 500 μl Buffer RPE is added onto the RNeasy MinElute spin column and centrifuged for 2 min at ≥8000×g (≥10,000 rpm). The collection tube is discarded with the flow-through.

The RNeasy MinElute spin column is placed into a new 2 ml collection tube and centrifuge at full speed for 5 min with an open lid to dry the membrane. The collection tube is discarded with the flow-through. RNA is eluted by adding 14 μl RNase-free water, let column stand for 1 min, and centrifuged for 1 min at full speed.

### Example 6: Tumor Educated Platelets (TEPs)

Tumor cells are known to transfer (mutant) RNA into blood platelets, which are subsequently used to detect tumor-associated RNA markers. This protocol minimizes degradation of platelet mRNAs and minimizes the contamination by other cell types like red blood cells and leukocytes (Amisten S, Methods Mol Biol. 2012). RNA obtained from blood platelets is then used to amplify and detect NRG1 fusions.

Preparation of Antibody-Conjugated Magnetic Beads: 1 ml Dynabead wash buffer (PBS with 0.1% (w/v) BSA, pH 7.4) is mixed with 250 μl Dynabead slurry (Dynabeads® Pan Mouse IgG, Invitrogen) and placed in the Dynamag magnet (DynaMag™-2 or DynaMag™-15, Invitrogen) for 1 min followed by removal of the liquid. After another wash, beads are removed from the magnet and resuspended in 250 μL wash buffer. 15 μl of anti-CD235a antibody (Mouse, anti-human CD235a, red blood cell surface marker, BD Inc, NJ, USA) and 15 μl anti-CD45 antibody (Mouse anti-human CD45, leukocyte surface marker, BD Inc, NJ, USA) are added to the beads, mixed and incubated for at least 30 min with gentle tilting and rotation. Supernatant is removed by placing the tube in the magnet for 1 min and beads are resuspended in 250 μl wash buffer before storing at 4° C.

Blood Collection and purification of platelets: A platelet inhibition cocktail is prepared with 18 ml ACD (sterile anticoagulant: citrate-dextrose solution, Sigma Aldrich), 12 μl of 1 mM PGE1 (Prostaglandin E1, 1 mM stock in ethanol, Sigma Aldrich), 120 μl of 30 mM acetylsalicylic acid (30 mM stock in ethanol, Sigma Aldrich), and 480 μl of 0.5 M EDTA (Sigma Aldrich). 8.5 ml of blood is collected in tubes containing in 15 ml tubes containing 1.5 ml platelet inhibition cocktail. After centrifugation at room temperature at 200 g, 20 min, top 85% of the upper layer, the platelet-rich plasma (PRP), is transferred to a new 15 ml tube. After another round of centrifugation (200 g, 10 min, room temperature) to further remove leukocytes and red blood cells, 85% of PRP is transferred to a 50 ml tube. The PRP is then filtered with the AutoStop™ BC high efficiency filter for leukocyte removal (Pall Inc, Cat. No. ATSBC1E), the filtrate is mixed with antibody-conjugated magnetic beads and incubated while tilting and rotating at room temperature for 45 min. The PRP/bead mix is placed in the DynaMag™-2 for 2 min and supernatant transferred to a new tube to obtain depleted PRP. After repetition of PRP depletion step, supernatant is centrifuged to harvest platelets at 800 g, 10 min at room temperature. Platelet pellets are weighed and dissolved using 1 ml TRIzol (Invitrogen) per 100 mg of pellet, with a minimum of 1 mL TRIzol for samples weighing less than 100 mg.

Isolation of platelet RNA: Platelet samples dissolved in TRIzol are incubated at room temperature for 5 min and 200 μl chloroform (Sigma Aldrich) added to each tube. After shaking vigorously for 15-30 times, tubes are incubated for 2-3 min at room temperature and centrifuged at 12,000 g for 15 min at 4° C. 10 μg ultra-pure glycogen (UltraPure™ Glycogen, Invitrogen, Cat. No. 10814-010) is added to new RNase-free tubes. The aqueous phase of the TRIzol/chloroform mix is carefully added to glycogen containing tubes followed by 500 μl of cooled isopropanol (Sigma Aldrich). The tubes are mixed and left overnight at −20° C. After centrifugation at 12,000 g for 15 min at 4° C., the supernatant is removed and RNA pellet washed in 1 ml of 75% ethanol. Dried RNA pellets are dissolved in RNase-free water and can be further used for reverse transcription into cDNA.

### Example 7: ddPCR Nucleic Acid Amplification and Fusion Detection

Droplet Digital Polymerase Chain reaction (ddPCR) is used to detect target nucleic acid fusions from liquid biopsy cfDNA and cfRNA samples.

Droplet Digital Polymerase Chain reaction (ddPCR) performed with Bio-Rad's QX100™ or QX200™ Droplet Digital PCR system allows to detect target nucleic acid fusions from liquid biopsy DNA and/or RNA (cDNA) samples.

Primers and Probes: Primers are designed around the breakpoints to amplify NRG1 fusion product of 60-200 bp in size. Primer3 program (Whitehead Institute for Biomedical Research, Massachusetts Institute of Technology) is used to design primers with a GC content of 50-60%, Tm between 5° and 65° C. (at 50 mM salt concentration and 300 nM oligonucleotide concentration), avoiding secondary structures and primer dimers.

TaqMan™ hydrolysis probes are used which are sequence-specific and fluorescently labeled with FAM, HEX or VIC dyes. The probe sequence is chosen to be in between the two primers of the amplicon. Tm of the hydrolysis probe is 3-10° C. higher than that of the primers and the GC content is 30-80%. The probes are <30 nucleotides long.

Sample preparation: Concentration of input DNA/RNA is assessed using A260 spectroscopy, to ensure that the target DNA/RNA concentration that is being loaded is within the dynamic range of detection. The recommended DNA dynamic range of the QX100 or QX200 System is from 1 to 120,000 copies/20 μl reaction. Restriction digestion is also performed for genomic DNA using a 4-cutter or a 6-cutter enzyme and 10 Units enzyme/μg DNA according to the manufacturer's recommendations. Digested DNA is stored at −20° C. until further use.

When the experiment is performed with cfDNA as the starting material, synthetic DNA containing the sequences of the fusion variants is used as the positive control. The fusion sequence includes the fusion site plus sufficient length flanking on each side to cover the PCR amplicon. The resulting nucleotide sequence is between 50-250 ntd. A T7 promoter sequence (5′-CAGAGATGCATAATACGACTCACTATAGGGAGA-3′) is added to the 5′ end of the target sequence. The synthetic sequences are ordered from IDT (www.idtdna.com) as double stranded deoxyribonucleic acid (DNA) fragments and reconstituted in Tris-EDTA (TE) buffer to a final concentration of 10 ng/μl.

When RNA is used as the starting material, RT-PCR is used to first generate cDNA, for instance with Bio-Rad's iScript™ Select cDNA Synthesis Kit for oligo (dT) or gene-specific priming. cDNA synthesis is performed according to the manufacturer's manual. The concentration of cDNA is reduced to about 0.2 ng/μl RNA equivalent and 5 μl is used per Droplet Digital PCR reaction (total volume 20 μl).

For generation of positive control for cfRNA, 60 ng of synthetic DNA is converted into RNA using in vitro transcription and the resulting RNA transcripts are purified using phenol/guanidine based reagents. DNase I is added to remove residual template DNA. Quality of RNA is assessed by gel electrophoresis. The obtained RNA is diluted to concentration ranging from 0.25 to 2.5 μg, based on desired copy number output in the test sample. 10 μl aliquots of analytical RNA for use in the positive control are stored at −80° C.

Nuclease-free water is used as negative control for both cfDNA and cfRNA. ddPCR reaction: 20 μl PCR mix is prepared, containing the sample nucleic acid (1 μg of DNA or cDNA with a final concentration of 50 ng/μl), 2×ddPCR supermix for probes ((Bio-Rad, cat. no. 1863023; without 2′-deoxyuridine 5′-triphosphate)-10 μl, 20× fusion specific primers/probes set (450 nmol/L primers, 250 nmol/L FAM probe)-1 μl, 20× control target primers/probe set (450 nmol/L primers, 250 nmol/L HEX probe)-1 μl and nuclease-free water. An initial reaction pool slightly more than 20 μl (22-25 μl) is created to ensure that 20 μl of mixture is transferred to the DG8 cartridge (Bio-Rad, cat. no. 1864007). Reaction mixes are combined and mixed well in a separate tube and not in the droplet generator cartridge. Reaction mixes are then transferred to the DG8 Cartridge already preloaded in the DG8 Cartridge Holder (Bio-Rad, cat. no. 1863051).

After loading the 20 μl PCR reaction, 70 μl of Droplet Generation Oil (Bio-Rad, cat. no. 1863005) is loaded into the bottom wells of the DG8 Cartridge. A gasket is attached across the top of the DG8 Cartridge which is placed into the QX200 Droplet Generator. The Droplet Generator produces about 20,000 droplets per sample in about 2.5 min for eight samples.

Droplets are then transferred to a 96-well plate by pipetting gently. The PCR plate is heat sealed using Bio-Rad's PX1™ PCR Plate Sealer and pierceable foil heat seal. The PCR plate is then placed in a C1000 Touch™ Thermal Cycler with 96-deep well reaction module for PCR. The thermal cycling conditions are as follows: Enzyme activation at 95° C. (10 min, 1 cycle); Denaturation (94° C., 30 sec) and Annealing/extension (55° C., 1 min) for 40 cycles; Enzyme deactivation at 98° C., 10 min, 1 cycle. A Ramp rate of 2° C./sec is used.

Following PCR amplification of the nucleic acid target in the droplets, the plate containing the droplets is placed in a QX100 or QX200 Droplet Reader, where using the QuantaSoft Software, each droplet is analyzed individually using a two-color detection system (set to detect FAM and HEX). Positive droplets, which contained at least one copy of the target DNA/RNA molecule, exhibit increased fluorescence compared to negative droplets. The concentration reported is in copies/μl of the final 1×ddPCR reaction. Thresholding tools are used to correctly designate the droplet populations as double negative (negative for both FAM and HEX), FAM positive, HEX positive, and double positive (positive for both FAM and HEX). Using the ABS analysis, absolute quantification of the target is obtained in copies/microliter and copies/droplet.

### Example 8: Anchored Multiplex PCR

Anchored Multiplex PCR (AMP) is used for the detection of gene fusions and to detect multiple fusions to the gene of interest. The following protocol is based on Archer FusionPlex Solid Tumor kit (ArcherDX, cat. no. AB0005) and performed using RNA obtained from liquid biopsy from e.g. example 6.

### Library Preparation:

A positive control is included which contains at least several confirmed gene fusions. A non-template control is included as an additional sample in every run.

Random Priming, First and Second strand cDNA synthesis: The desired input for the assay is 200 ng RNA, dilute in 10 mM Tris HCl pH8.0. 20 μl of the diluted RNA is added to the pre-chilled Random Priming reagent strip tubes (provided in the kit). Following mixing and a brief spin, the mixture is transferred to 96 well PCR plate, sealed with RT film (USA Scientific, cat. no. 2921-7800) and incubated in thermocycler block at 65° C. for 5 minutes (heated lid).

The random priming product is transferred into the First Strand reagent strip tubes (placed in pre-chilled aluminum block) and mixed, brief spin and transferred to a 96 well PCR plate. The following thermocycler program is used: 25° C. 10 min, 42° C. 30 min, 80° C. 20 min, 4° C. hold (heated lid).

1:10 dilution of the first strand product is made in a new set of PCR strip tubes in nuclease free water, to be used in Pre-Seq quality control (QC) assay. QC assay is primarily used for the verification of no cDNA synthesis from the no-template control. The QC assay is carried out according to the manufacturer's protocol.

For second strand cDNA synthesis, 21 μl nuclease free water is added to the remaining first strand product and 40 μl of this product is added to the Second Strand reagent strip tube (provided in the kit, placed in pre-chilled aluminum block). After mixing and a brief spin, the mixture is transferred to a 96 well PCR plate and sealed. The plate is inserted in thermocycler block, and the following thermocycler program used: 16° C. 60 min, 75° C. 20 min, 4° C. hold (heated lid).

End Repair: 40 μl of the second strand product is transferred into the End Repair reagent strip tube (provided in the kit, placed in pre-chilled aluminum block). After mixing and a brief spin, the strip tube is incubated in thermocycler block at 25° C. 30 min followed by 4° C. on hold. The End Repair product is added to purification beads (Agencourt AMPure XP Beads, Bechman Coulter, cat. no. A63881) at room temperature. After mixing, the mixture is incubated at room temperature for 5 minutes, followed by a 5-minute incubation on magnet (Alpaqua, cat. no. A32782). Supernatant is discarded, beads are washed twice with 200 μl 70% ethanol with 30 second incubations and air dried for 5 minutes. The beads are re-suspended in 22 μl 10 mM Tris HCl pH 8.0, incubated off the magnet for 3 minutes followed by a 2-minute incubation on the magnet.

Ligation Step 1:20 μl from the End Repair bead purification plate is transferred into the Ligation Step 1 strip tubes (provided in the kit, placed in pre-chilled aluminum block). After mixing and a brief spin, the mixture is incubated in a 96 well PCR plate in thermocycler block for 37° C. 15 min and 4° C. on hold (heated lid). The entire volume of Ligation Step 1 product is added to 50 μl of room temperature equilibrated beads. After mixing, the mixture is incubated at room temperature for 5 minutes, followed by a 5-minute incubation on the magnet. The supernatant is discarded and beads washed twice with 200 μl 70% ethanol with 30 second incubations. After the final wash all 70% ethanol is removed and the beads air dried for 5 minutes. The beads are removed from magnet and re-suspended in 42 μl 10 mM Tris HCl pH 8.0, incubated off the magnet for 3 minutes followed by a 2-minute incubation on the magnet.

### Ligation Step 2:

MBC Adapter Strip tube reagents (provided in the kit) are removed from 4° C. storage and numbered properly. The sample-specific indexes are also recorded for sequencing purposes.

40 μl from the Ligation Step 1 bead purification plate is transferred to the MBC Adapter strip tubes (ArcherDX cat. no. AK0016-48, placed in pre-chilled aluminum block). After mixing and a brief spin, the mixture is transferred to Ligations Step 2 reagent strip tubes (provided in the kit, also placed in pre-chilled aluminum block). After mixing and a brief spin, the mixture is incubated in a thermocycler for 22° C. 5 min, and 4° C. on hold.

Ligation Cleanup beads (equilibrated at room temperature) are vortexed and 50 μl added to a new set of PCR strip tubes. After 1 minute incubation on magnet, the supernatant is discarded. Strip tubes are removed from magnet and re-suspended in 50 μl of Ligation Cleanup Buffer.

Ligation Step 2 product is added to the Ligation Cleanup bead strip tubes, mixed by vortexing and incubated at room temperature for 5 minutes. The mixing and incubation is repeated following which the samples are incubated on the magnet for 1 minute and supernatant discarded. 200 μl of Ligation Cleanup buffer is added for re-suspension and after a quick spin and the mixture is placed on magnet for 1 minute. Washing with the Ligation cleanup buffer is repeated. An identical wash is performed using ultrapure water, following which the beads are re-suspended in 20 μl of 5 mM NaOH and incubated in a PCR plate as follows in thermocycler block: 75° C. 10 min, 4° C. hold (heated lid). The PCR plate is placed on magnet for at least 3 minutes.

First PCR: 2 μl of the NRG1 specific primers (GSP1 primers) are added to each well of the First PCR reagent strip tubes (provided in the kit) and mixed with 18 μl of Ligation 2 cleanup product. After a brief spin, the mixture is incubated in a thermocycler with the following program: 95° C. 3 min, 15 cycles 95° C. 30 sec-65° C. 5 min (100% ramp rate), 72° C. 3 min, 4° C. hold (heated lid).

20 μl of First PCR product is added to 24 μl of room temperature-equilibrated beads, mixed and incubated at room temperature for 5 min and on magnet for 2 min. Following removal of supernatant, the beads are washed twice with 200 μl 70% ethanol with 30 second incubations, air dried and resuspended in 24 μl 10 mM Tris HCl pH8.0.

### Second PCR and Library Quantitation

2 μl of the additional NRG1 specific primers (GSP2 primers) are added to each well of the properly labeled Second PCR reagent strip tubes (provided in the kit) in a cold aluminum block, and mixed with 18 μl of the First PCR cleanup product. After a brief spin, the mixture is incubated in a thermocycler block as follows: 95° C. 3 min, 18 cycles 95° C. 30 sec-65° C. 5 min (100% ramp rate), 72° C. 3 min, 4° C. hold (heated lid).

20 μl of Second PCR product is added to 24 μl of room temperature-equilibrated beads, mixed and incubated at room temperature for 5 min and on magnet for 2 min. Following removal of supernatant, beads are washed twice with 200 μl of 70% ethanol with 30 second incubations, air dried and resuspended in 24 μl 10 mM Tris HCl pH8.0. After 2 minute incubation on the magnet, 20 μl of Second PCR cleanup product is transferred to a new PCR plate and quantified.

The concentration of each library is determined by qPCR using Kapa Biosystems Library Quantification Kit for Illumina no. KK4973, according to the manufacturer's instructions. The Second PCR product is serially diluted using 10 mM Tris HCl pH8.0 0.05% Tween. 6 μl of library quantitation master mix (Kapa Biosystems, cat. no. KK4973) is added per well to an optical 96-well plate followed by 4 μl of appropriate dilution or Standard (Kapa Biosystems, cat no. KK4906, KK4903). The following qPCR program is used: 1 cycle 95° C. 5 min, 35 cycles 95° C. 30 sec (4.4° C./sec ramp rate)-60° C. 45 sec (2.2° C./sec ramp rate). After library quantification is complete, all libraries are normalized and pooled by combining 10 μl of each normalized library into one 1.5 ml microcentrifuge tube.

### Sequencing of Libraries:

Sequencer reagent cartridge is removed from cold storage and placed in deionized water up to the fill line for at least one hour. The sequencer reagent kit (MiSeq Reagent Kit v3-600 cycle; Illumina; cat. no. MS-102-3003) is allowed to equilibrate with room temperature. 10 μl of the library pool is combined with 10 μl of 0.2N NaOH to make the Denatured Amplicon Library (DAL) pool and incubated at room temperature for 5 min. 10 μl of 200 mM Tris HCl pH 7.0 is added to the DAL, followed by 970 μl of HT1 buffer (provided in the kit).

The final load tube is made by combining 300 μl of HT1, 25 μl of 20 μM PhiX and 675 μl of the DAL pool. After mixing and a brief spin, the entire volume of the load tube is added to the sample well of the sequencer reagent cartridge and the cartridge is loaded into the sequencer running 2×151 bp reads with 2×8 index reads (MiSeqDx System-Illumina). RNA Fusion data is analyzed using an appropriate bioinformatic data analysis software.

### Example 9: Next-Generation Sequencing

Next-generation sequencing is performed with DNA samples using MI-Exome test (Caris Molecular Intelligence®, Caris Life Sciences) that uses DNA and provides tumor mutational profiling using a custom human exome panel to detect variants including single nucleotide polymorphisms, insertions/deletions and DNA rearrangement and fusion events among others.

The following custom exome panel, which is a blend of five panels is used. The panel is validated for use with the Illumina NovaSeq 6000 instrument. The blend includes two off-the-shelf panels from Agilent and three panels developed and optimized by Caris:
Agilent Human All Exon V7 Panel (48 MB)
Agilent SNP Backbone Panel (3 MB panel with 1 MB resolution)
Caris 719-Gene Targeted Clinical Panel (1 MB)
Caris Intronic Fusion Panel (0.1 MB)
Caris Pathogenic Panel (0.1 MB)

For RNA as the starting material, next-generation sequencing is performed using a whole transcriptome assay, for instance the MI Transcriptome™ assay (Caris Molecular Intelligence®, Caris Life Sciences). The assay detects gene fusions and splice variants using the Agilent SureSelect Human All Exon Panel when sequences on the Illumina NovaSeq instrument.

### SEQUENCE INFORMATION

### NRG1

NRG1 sequence information, providing nucleotide sequence of exons 1-13 as indicated by alternating underlining, starting with exon 1 underlined, exon 2 not underlined, exon 3 underlined, exon 4 not underlined, and so forth.
SEQ ID NO:1=Exon 1
SEQ ID NO:2=Exon 2
SEQ ID NO:3=Exon 3
SEQ ID NO:4=Exon 4
SEQ ID NO:5=Exon 5
SEQ ID NO:6=Exon 6
SEQ ID NO:7=Exon 7
SEQ ID NO:8=Exon 8
SEQ ID NO:9=Exon 9
SEQ ID NO:10=Exon 10
SEQ ID NO: 11=Exon 11
SEQ ID NO: 12=Exon 12
SEQ ID NO: 13=Exon 13
SEQ ID NO: 14=Exons 1-13
SEQ ID NO: 15=Exons 6-7
SEQ ID NO:16=Exon 2-13
SEQ ID NO: 17=Exon 6-13
SEQ ID NO:18=CAT of Exon 5+ exons 6-13 (i.e., CAT plus SEQ ID NO: 17)

SEQ ID NO: 14
AGTAAGCCTCCGCAGCCCACTCGGACTGCAGCCTGTTTGCCGCCC
GTCCTCCCATTGCAGCACTCGGGGCGACAGAGAGGGAGGAGGCGC
GCGGGGACGGGGACGCCCAGGAGGACCCACTCGCGGGTCCCGCTC
CGCTCCGGCAGCAGCATGGGGAAAGGACGCGCGGGCCGAGTTGGC
ACCACAGCCTTGCCTCCCCGATTGAAAGAGATGAAAAGCCAGGAA
TCGGCTGCAGGTTCCAAACTAGTCCTTCGGTGTGAAACCAGTTCT
GAATACTCCTCTCTCAGATTCAAGTGGTTCAAGAATGGGAATGAA
TTGAATCGAAAAAACAAACCACAAAATATCAAGATACAAAAAAAG
CCAGGGAAGTCAGAACTTCGCATTAACAAAGCATCACTGGCTGAT
TCTGGAGAGTATATGTGCAAAGTGATCAGCAAATTAGGAAATGAC
AGTGCCTCTGCCAATATCACCATCGTGGAATCAAACGAGATCATC
ACTGGTATGCCAGCCTCAACTGAAGGAGCATATGTGTCTTCAGAG
TCTCCCATTAGAATATCAGTATCCACAGAAGGAGCAAATACTTCT
TCATCTACATCTACATCCACCACTGGGACAAGCCATCTTGTAAAA
TGTGCGGAGAAGGAGAAAACTTTCTGTGTGAATGGAGGGGAGTGC
TTCATGGTGAAAGACCTTTCAAACCCCTCGAGATACTTGTGCAAG
TGCCCAAATGAGTTTACTGGTGATCGCTGCCAAAACTACGTAATG
GCCAGCTTCTACAAGCATCTTGGGATTGAATTTATGGAGGCGGAG
GAGCTGTACCAGAAGAGAGTGCTGACCATAACCGGCATCTGCATC
GCCCTCCTTGTGGTCGGCATCATGTGTGTGGTGGCCTACTGCAAA
ACCAAGAAACAGCGGAAAAAGCTGCATGACCGTCTTCGGCAGAGC
CTTCGGTCTGAACGAAACAATATGATGAACATTGCCAATGGGCCT
CACCATCCTAACCCACCCCCCGAGAATGTCCAGCTGGTGAATCAA
TACGTATCTAAAAACGTCATCTCCAGTGAGCATATTGTTGAGAGA
GAAGCAGAGACATCCTTTTCCACCAGTCACTATACTTCCACAGCC
CATCACTCCACTACTGTCACCCAGACTCCTAGCCACAGCTGGAGC
AACGGACACACTGAAAGCATCCTTTCCGAAAGCCACTCTGTAATC
GTGATGTCATCCGTAGAAAACAGTAGGCACAGCAGCCCAACTGGG
GGCCCAAGAGGACGTCTTAATGGCACAGGAGGCCCTCGTGAATGT
AACAGCTTCCTCAGGCATGCCAGAGAAACCCCTGATTCCTACCGA
GACTCTCCTCATAGTGAAAGGTATGTGTCAGCCATGACCACCCCG
GCTCGTATGTCACCTGTAGATTTCCACACGCCAAGCTCCCCCAAA
TCGCCCCCTTCGGAAATGTCTCCACCCGTGTCCAGCATGACGGTG
TCCATGCCTTCCATGGCGGTCAGCCCCTTCATGGAAGAAGAGAGA
CCTCTACTTCTCGTGACACCACCAAGGCTGCGGGAGAAGAAGTTT
GACCATCACCCTCAGCAGTTCAGCTCCTTCCACCACAACCCCGCG
CATGACAGTAACAGCCTCCCTGCTAGCCCCTTGAGGATAGTGGAG
GATGAGGAGTATGAAACGACCCAAGAGTACGAGCCAGCCCAAGAG
CCTGTTAAGAAACTCGCCAATAGCCGGCGGGCCAAAAGAACCAAG
CCCAATGGCCACATTGCTAACAGATTGGAAGTGGACAGCAACACA
AGCTCCCAGAGCAGTAACTCAGAGAGTGAAACAGAAGATGAAAGA
GTAGGTGAAGATACGCCTTTCCTGGGCATACAGAACCCCCTGGCA
GCCAGTCTTGAGGCAACACCTGCCTTCCGCCTGGCTGACAGCAGG
ACTAACCCAGCAGGCCGCTTCTCGACACAGGAAGAAATCCAGGCC
AGGCTGTCTAGTGTAATTGCTAACCAAGACCCTATTGCTGTATAA
AACCTAAATAAACACATAGATTCACCTGTAAAACTTTATTTTATA
TAATAAAGTATTCCACCTTAAATTAAACAATTTATTTTATTTTAG
CAGTTCTGCAAATAGAAAACAGGAAAAAAACTTTTATAAATTAAA
TATATGTATGTAAAAATGTGTTATGTGCCATATGTAGCAATTTTT
TACAGTATTTCAAAACGAGAAAGATATCAATGGTGCCTTTATGTT
ATGTTATGTCGAGAGCAAGTTTTGTACAGTTACAGTGATTGCTTT
TCCACAGTATTTCTGCAAAACCTCTCATAGATTCAGTTTTTGCTG
GCTTCTTGTGCATTGCATTATGATGTTGACTGGATGTATGATTTG
CAAGACTTGCAACTGTCCCTCTGTTTGCTTGTAGTAGCACCCGAT
CAGTATGTCTTGTAATGGCACATCCATCCAGATATGCCTCTCTTG
TGTATGAAGTTTTCTTTGCTTTCAGAATATGAAATGAGTTGTGTC
TACTCTGCCAGCCAAAGGTTTGCCTCATTGGGCTCTGAGATAATA
GTAGATCCAACAGCATGCTACTATTAAATACAGCAAGAAACTGCA
TTAAGTAATGTTAAATATTAGGAAGAAAGTAATACTGTGATTTAA
AAAAAACTATATTATTAATCAGAAGACAGCTTGCTCTTACTAAAA
GGAGCTCTCATTTACTTTATTTGATTTTATTTTTCTTGACAAAAA
GCAACAGTTTTAGGGATAGCTTAGAAAATGGGTTCTGGCTTGCTA
TCAGGGTAAATCTAACACCTTACAAGAGGACTGAGTGTCACTTTC
TCTCTGGGGGAATGATCCAGCAGCTTATCTAGTTGACAATCAAAA
CACGGCTGATAAAGGTGCAATCATTTCTGACATGTATTTTTCACT
GATTTTGAAGCTAGTGATTGGTTGTGTCTTCTTGGCTCAAAAAGA
AGCATATTACGGCACAAAAAGCCCAGCCCAGACAGCACATGCAGC
ATTTTGTCTGAAATACTTCTAGAGTCAAACGTGCCTGCTGTACAT
AGCGATGACTTGTCATCATAGGGAAGTATTTCCATCGTAGAGTGT
TCAGAAGGAGTGACTGTATAGGTGGAGAGAAGCTTAGTGACTCCG
TTGAAATTTTAAAATGTGGATGACCACCCCTTTCTCCCCCTTATT
TTTCTTTTATCTTTCCATGTTGCCTTGATCAGGTCATAACTATGC
ATGAACATTTTTTATCAGGAATGGCCGATGTGTATGTGATTTGTA
ATCACAAGTAATGATTCATCAGGAAATGTCAATCCTGTTGGAAAG
ATTGCACCTTTACTTGCAGAAGTGACCCCCACCTGTGTCCTGACC
TCTCCATTTACAGGCTCTCTCACCCATTTCCCCCACCTCCTTTAA
TTTTTGCTTTACTGTCATAAAGTAGGACTAAGATTGGTCTAAGCA
TTGCATGTTCTTTTGTGATGGTAAATCCAAAGGAAGGCCTATAAG
TATTAACATTTGAAATAACTGCTAATTCAGGAAAATGGAAGAAAA
AAAATTATTTGAAACACAGAACCCATTTCATGGCCTGCCTGATAT
CTGTGAAATCAGGGCTGGAGCTTTACTTAGGATTCACATGGCCTC
CTAGGAACCATGGGACAAATGGGAAACAGGTTATCGGGGGATTCA
TGAAGTCAGTGAGAGTAATTGCTTCTTTTTTGCGGGTGAACTGAA
TGTATTTCTTCACCAAATCTTGATGTTAACAATTAAAAAGAAGAA
ATGACATGCAAGTAGGTCTTAGCAGAAAAATGCAGGCTGGGCATG
AGTCATGTTGTTACCCTCCCACATGCTCCTACAATCCACAGAGAT
GCCTGTCTGCAGGTTCTTGAAGTTATTGTTAGTATTTGGTATCTC
AAATTTTTCGTCACTGTTCACATGCCACTTTCTCTGTGCACAGTG
GTATCCTCATTTGCTTTTTAACCTACACTGAGGAGTCTTTGTCAG
GTTGCACTGATTTTCCAATTCTGCAGTAATGAGTAAGCTCACGGC
ATGGGGAAGAAGACAGTCAGTCCAATGAAGTTCTCTAAATTATTT
TAACATTGCCTTTGAAGGCCTTGACTCATCCTTAGCTATTTCAAT
GAAGAAATTCCTACCATGAATTTAAAACCCTAAAAATTCTGTTTC
AAATTCTTTGGGCATTGGGGTACTCAGATATCCCATTGTGGAAGA
ATTTTAAGAATAAATAGAAGTTTCTGTTGAGAACCATGAGCAACA
TGTTTCTTACAATGAGAATTGCTATGCATTTTAAAATTGCAAATA
TATATGAAAATTGAAGACAAGAGGAAATTGTATTTCTAACTTGAT
TCTGATCACTCACAGAGGTGGCATATTATTATAGTTGGGACATCC
TTTGCACCCTTCATAAAAAAGGCCAGCTGACTGCTCAGCATCACC
TGCCAAGGCCACTAGATTTGTGTTTACAGGGGTATCTCTGTGATG
CTTGTCACATCACTCTTGACCACCTCTGTTAATAAATTCCGACAG
TGCAGTGGCGATCGGAGTGTGAACTTATGTTCCCAGCATATGGAA
AGCTATCTTAGGTTTTAAGGTAGTAGAAATTGCCCAGGAGTTTGA
CAGCAACTTTGTTTCCCGGGTCTAAAATCGTATCCCACTGAGGTG
TATGCAGTGGAGCATAATACATGCAAATACATGCAAAACTCCTTT
TGTTTCACCTAAGATTCACTTTCTATCTTACTTTCCCTTCCTGCC
TAGTGTGACTTTTGCCCCCAAGAGTGCCTGGACAGCATTCTAGTT
TCTACAAAATGGTCCTCTGTGTAGGTGAATGTGTCCCAAACCTGC
TATCACTTTCTTGTTTCAGTGTGACTGTCTTGTTAGAGGTGAAGT
TTATCCAGGGTAACTTGCTCACTAACTATTCCTTTTTATGGCCTG
GGGTTAAAGGGCGCATGGCTCACACTGGTGAAAATAAGGAAGGCC
TGGTCTTATCTTGTATTAATAATACTGGCTGCATTCCACCAGCCA
GAGATTTCTATCTGCGAAGACCTATGAAACACTGAAGAGAAATGT
AGGCAGAAGGAAATGGCCACATATCACAAGTTCTATTATATATTC
TTTTGTAAATACATATTGTATATTACTTGGATGTTTTCTTATATC
ATTTACTGTCTTTTTGAGTTAATGTCAGTTTTTACTCTCTCAACT
TACTATGTAACATTGTAAATAACATAATGTCCTTTATTATTTATA
TTTAAGCATCTAACATATAGAGTTGTTTTCATATAAGTTTAAGAT
AAATGTCAAAAATATATGTTCTTTTGTTTTTCTTTGCTTTAAAAT
TATGTATCTTTTCCTTTTCTTTTTTTTAAGAATAATTTATTGTTC
AGGAGAAAGAATGTATATGTAACTGAAACTATCTGAAGAATGCAC
ATTGAAGGCCGTGAGGTACTGATAAACTAAAGAATTTATTATTCA
AAATACTAAGCAATAAGTAATTGTGATTTATTTAAAGTTTTGTCC
ATTTTCCATGAAAGACATACTGCAATAAAAATGCTACTCTGTGGA
GACCTGGGAGTGTTGCTCAGCAGACTACAGCTTCAGTCTGTTAGA
CCAGCACCTTTCATCTCATTCCCATAGTTATGCTAATTTAGGATT
GTGTTCCATGGACCCCATGATCACCTTGTCTATACGTTGCTTCTT
GTCTGTCCATTGCTTTTGCCACACCACCTGTTCTCAAATCATCTC
CTCCCTACCAATGCTGTTTATCACTTTCTTCCTTGTTGAAGAGGC
CACACAACCAGACAGTACTATGCTTCCTTTTTCCTCCATACACAA
TAACAGAGAGAGAATATTCTAGGGCATGACTGCCTGGATCCTGGC
TGTTGCTATCTTTTGTAGTGGCAGTAAGAAACTCCTTCAGACTAA
TGAAAATGTCAACGTGCCATTCAATCACGAAAGGTAACGAAAAAT
GCTCTCATGGTTCAAATAGTCCAATGGCCCATAGTGGCCTAAAAG
GCAGCCAGTTGACACCTGGCCATGCTAAGCTTCCTTATACCATCC
GCTAATGACTTTCCATTGGGCCCACAATTTACGGATTCATAATTT
TAAAAGAGGAGAAGGCCAAGTTAGGTTCATTCCCCTTATTCTGTC
AATAAAACAAATCAAACTCATGTCTATCTAACTGCTCAGGGAGGA
GCCTTTGCATGAGAAAATTCTCATATTCTAAGACTGAGTCATAGA
AATGAGGGTATTACTTTTCTTACTGCAATTAACCTAAACAAAAGC
CATATTTTAACAAATAGATATTTGCATGGTACCCTTCATATATTC
CAAGCATTTCACTCATTATTCCAGGTAGGTTAAGAGCTTCTGAAG
TGTATGAAGTAAAGGTCAGCAATCCTTTGGGGTGAACAGTGGCCT
CCTTTGGAGTTTGGGGGTAACCTGAGACTTCCCACCAATGTCCAC
CTCCATCTGTGTACCTAATTCCTATTACCTAGTTATGGCTCCTCT
AGGATCATTTCCAAACACTCTGGATGTCCAGGAAATTTAAATTGT
AGCTTTTGACTGAGCTAGTTTTTCCTATTTATATTAATAAATTTT
CAAAAATGCTTGAAATCTTCACATTTGCAACAACTTTAGTTTTCA
TGCACATACAAACACAGAGAGACAAAAATTCCAAACAGACACTCT
CCAAAAGCCACCACACTCTTTCACTTGCTCTATAGTCATTTAGCC
AACCAGCCATGCAGAGAATATTTAAAACTTAAAGATTGAGACATT
ATTCTCAGTTTTTGCTGAGGCTTTGTAACGAAATTGAACACTATA
AGCAGCTATTGTAGTAATTTTGGTTAAAATTGTTTGCCTGGGATA
TAGTATTTGAGGCAGAAGCACGTGTGTGAAGGAGGTGAGGTGGTT
TGGAAAGAGTGAAGACTCGCAGCCAGATTGAATGTCTGGATAATT
ACTATAATTCTCCCTTCTTGGTTGAAACCATGTTCTCTCTTGATT
TTTAAACCCAGGCTGCCTCTGGAAACAAGCAAACCTGAGTCTTTC
TAACCTGAGTCTTCCCAATCATTAGATTTCTTTTCTGTCCTAACG
ATGAATGATAAAAGGACTTGATGTTCACAATTTGGGGTTATAAGG
CAGGTCTGAAATCTGGAGACTCAAGATGCTGGAAGGAGTGGAAAG
TTTCGATGACTTTATATGAATCACTTTGCACTCTATGTTTGGCTT
GTCCTCTTTGAAACTGATTTACTAAAATAAATGTAAGGGAACTAT
TACTCCAAAAGATTAACTTGGCAGGAAATACCAATACTTTCAGTT
TATGAAAGACAAAACTGTCTTGTTGCTACAGGAAGCTGCAATGTT
CCTAACCTTTAAGGTTGGTGTTGAATAGGGTGGTCATGCCCTCCC
CTGCAGGTATCTTTAGGCTCCTGTTGACCTCCTGGTACTATAACT
GTTCGTCTTCTCTGGGTAGCTATTGATTTTGAACTTTAACATGCT
TCAAAACTTTATTCATCAGGGAAATAGGAAAAGAGTTTTGTTACC
TGGAGGAAATCTATTGTGATCTACCTGAGCTTTTTAAAAACAGAC
CAGGAGAAGGAAACCAGTAATTTTTAAAGAAGAGACAGAGAATGG
GATAATAGTTTCACCCAGGATCTCTTTCTAACCCTTTCCCTTCAA
ATGAACTTATTGGAACAGAATTGGAAAGAAGAAAGGACATCTCTG
CCCACCCCACAGGATGCCAAAAAGGCTAAAGAATTACCTCTGTAG
ATTTAAACATCTTTAATGGCTTATGTATAGATTTGCTAATACAGA
GAGAAATGAACTATTAAATAAAAATCACATTTTATAATATTTTTA
TGGCTTAAAACATCCTTTATCTCCTTTTTGTTCTCTCTACATGAT
ATGGTAAGTGATGAGGAAAATTTAGGCTCAGGAAGGTTAAAATCT
TTCTTGGAGTTACACATCTAAGAGAGCTGCAGAGCTGACACTTGT
ACCCAGGTTTTCTGACTGCAAATCCAGTTTCTTTCTATTGCGTTC
TTCCCCTTTCCCTGCCTCAAGCAGAAACAGGTTTTTTATTTTCAA
CCTTTATGTATACAGTATGTTATGTTACATCTACAGCTAAGTTTC
TTTTTAGAAGAATGTGAGCCCTTCTAGCTTTGGTTTAGAGTGATT
CTAGAAGCCAATTTCCTTGGCTTAGTGATTCTATGCACCTTTCCT
AAACTTAGCTTTCTAAGGAAATGAAGTGTACGAGTGAGAATGAAT
TCACAATTTCGACATGTAGGTAGCATCCTAAAGTGAAAAGAGGAG
GAAATTTGTGGTCAAAGCACTCTCCCCACCACTTAGAAACTTACT
GACTGTGGGCAGCTTCCTCCTCCAAGTTTCCTTCCTGATTTACAA
GACCGTGGTGTGGTCAGGATTAAACTTGAATACATGTAAGGAAGC
CTGAAAGTGTCTAACACATAGCGAGTATTCAAATGCCACCTTCTA
TTTGATCCTTCCCCTCCAGTTCCTTAAGTTTTGGAATCTAGGTTT
CTCAGTTCCAAATGGATTGACATTTGCATATCCCCATTGCACAAT
GGATCAAATAAACTTTATGTTATCATTTCTCCAACATAGTGCCAG
TAAGCAAATCCTTTTTAATAACAACAGTATGTTGAGAAACATATC
ACCAAATAATATTTAACTTTGTAGCTTTGATAAGTTCTTTAGGTT
TTGGTTTTGGTTTTGTTTTCTGAGACAGGGTCTTGATCTGTCACC
CAGACTGGAGTGCAATGGTTCAATTTTAGCTCACTGCAACCTGTA
ACTCCTGAGCTCAAGTGATCCTCCCACCTCAGCCTCTCAAGTAGC
TGGGACTACAGGTGTGCTCCACCATGCCCAGCGATTTTTTTTTTT
TTTTTTTTTTTTTTTTTTGGTAGGGACAAGGTCTCGCTATGTTGC
CCAGGCTGGTCTTCAATTCCTGGCCTTAAGTGATCCTCCTGCCTC
GGCCTCCCAAAGTGCTGGAATTACAGGCATGAGCCACCACCCATA
ACTTTATGTTTGTTTTTTTGATGCAGTATAAGTTCAGCTTGCTTC
TTATGCAGCCATACCATTTCATGTTAACTCTGATTTTTAGCAGCT
TATTACATTAGTGTTTTATTATTAATATAATTTTACAGAAATTTA
CTAAACCATGACTCTGTAGAGTTTTAATAATACTACCTCCAAACA
TCATTGCAAACATCTAGAAGAATGAACAAAAATGATCTTAGATCG
ACAGTATATCTGTTTGTCTTAGTTTCTACACAGGATGTTCAGACA
TATTCCATTTCTTTAAAAAAAAAATATATATATATATATATATAT
ATAGGCCTGGCACGGTGGCTCATGACTGTAATCCCAGCACTTTGG
GAGGCTTAGGCAGGCAAATCACCTGAGGTCAGGAGTTTGAGACTG
GCCTGACCAACATGGTGAAACCTCGTCTCTATTAAAATTACAAAA
ATTAGCCGGGCGTGGTGGCACATGCCTGTAATCCCAGCTACTCGG
GAGGCTGAGGCAGGAGAATCACTTGAACCTGGGAGGCAGAGGTTG
CAGTGAGTTGAGATCACGCCATTGCACTCCAGCCTTGGTGACAAG
AGTGAAACTCCGTCTCAAAAAAAAAAAAAATATGTATATATATAT
ATATATATATATATATATATATATATATATATATATAAAATCCCA
CCAAAAGTCTGCAGAGTGACCAAATTAGACGGCTCTGGTTTCAGA
TTAAATTCTAAATGTGAGAAACCACATAGCTCCCATGATCCATCC
AATAATTCCTCACGTCCTCTTCACTCTTTACTCCATGCATAAAAC
AGAATTTTTTTTTCTCATCCTGGGTATGAAGCAATTAATAATTTA
CGGATTTAGCCTATTTGGATTCAATCCCTTCAAACTCCATACTAC
ATCCAAGGTGGAAGTGACTTAAACTCTGATATCAATCATCAGGCT
TGTAATATAGGCTTTGTTAATGGCAGGAGAGTCTAATAAAACTTT
CTGTTCCTTATCCTTCATTTAAATGAAAAACTTTTTATTGAAAAC
AATCATAACTCTAGCTCATCATAAATATAATTCATGAGGACATTT
TATTATTTTTATATTAAAGAAATAATATTATAGATGTAAACTTTG
CACCTTTCTAATTATTATCATGAGTTAAGCTAATACTTGTCTTCT
GGTCCCTAGATGATGATTCTTTTTTGCCTTACTGGAGGAGCCCTT
GTCTTGAAGTGAGTTGCTTCAACAGCAGAGGACTTCTAGTTTCTC
CCAGTTGAGCCTAAAGTGAACTTTTCATCTTCTTCAGAGGAAGGG
GCTTCCTTGATTTGTACTTTTGTGGCTCCTCAGATAACACAGACA
ATTTTATCTTGGATCCCAGGTTCTCTTCACCATTAAGAATAAGAA
AGAGAGAAAATGCTGTGCATGACAGCCACCTACTCCAAACTACCC
AACCCCCTGTAACCAGGTACCTTCCAACAACGAGATGATTCTGCC
CTCACTCAAGAGTCTCCCCCACAAAGATTCCATTCTCCCTTTACT
TTTTATTTTTTATTTTTTTGCAAAACAAAGGCCTCCTTTAGTACC
TCCTTAGTTTATAACCCTTATCTTCCCAGCTCTTCCCTTCACTGA
TACCTCTGATTTCAAAAGTTCTGAAGTCGGAAGACCACACAATTT
CAGACTGTGAACAGAAATTCAGTCAGAAATTATTGGAGTTAGAAA
GAATTTAGAGAAATTGTATTGAACTAGAAAGTCCTCTTTGATTAG
TGGTGGCCCTTTAGAAAGTTCTAGGGCAGAGTCCCATGGTGTTTC
ATCTTTTCCATGATTTGCTTGACCAAAAACTTTTCTTTCACAACA
TGAAAATATGCTAATCCACCACACATTTTGGATTCTGCTCTGTTT
GCCTGAGGTGTTAGATCTCTAGCCAGGACTGTGAAGGGAAGGAAC
TTGAATCCTTCCTATTGAGCTATTAATGCAGAGTCAGTGAGATGA
AGGGTTCCACTCGGGGTCAAAATCATGTCAGTTACCAAGCAAAGG
AGCAAGTAAGGGGAAACATCTCCTCATCTGGTTAGTGGAGCCACA
TTTCACCCACTGATCAAGCCAGACCTGAGCAATAGTCTAGATTTC
TCCCTCCACATCTAATTGGTGACAATGGTGATTGTACCACTGACA
GGTCACACAAGTCCACACCCTCCTTCACAGCCTCACTGCTTCGGC
TCTTGTTTATGCTCTTATCAGCATCTGTCACTAGGATCCATTTGT
CTCCTGACTCATCTACTTCCTTTCACTCCCCTGGGCCATCCTTCA
CATCATGCTAGAAGACTGTGTCTAACTTGCAGAACTTATTGTGTC
AATCTCCTGGTTACGGCCCCTCCATGGCTCCCCACCTGACATAGG
AGGCCCTTCACAATCTGGCTTCTGTCACTCATAACTTGTCTCCAG
CCTTCATTCTTCAGTCTGATTTTATGGTTTTCTAGTTCCCCAATA
CACCACACCAGTGTTTATGAAACCCTAGTCATTGGCCTACGAACT
TTATGATTATTGACATATTCATGTACCACCTGTATTATTTTTTGC
ATAGTGTTTATTTTTAATTGACTACATTTTTAACTACAATAAAGT
AATTTCAACTAAAA

### VAPB

VAPB-NRG1 fusion sequence
SEQ ID NO: 19
CAGGTCCTGAGCCTCGAGCCGCAGCACGAGCTCAAATTCCGAGCC
TTGCCTCCCCGATTGAAAGAGATGAAAAGCCAGGAATCGGCTGCA
GGTT

VAPB sequence information, providing nucleotide sequence of exons 1-6 as indicated by alternating underlining, starting with exon 1 underlined, exon 2 not underlined, exon 3 underlined, exon 4 not underlined, and so forth.
SEQ ID NO:20=Exon 1 VAPB
SEQ ID NO: 21=Exon 2 VAPB
SEQ ID NO: 22=Exon 3 VAPB
SEQ ID NO: 23=Exon 4 VAPB
SEQ ID NO: 24=Exon 5 VAPB
SEQ ID NO: 25=Exon 6 VAPB
SEQ ID NO:26=Exons 1-6 of VAPB

SEQ ID NO: 26
AGTCCCCGCCCCTGGAGCCGGCGGCGCAGGGCGCAGCTTCCCGCC
GCCAGAGCGGGCCAGCCTGCTGCGTGCGTGCGTGTGTACGACTCT
GCGTGCGTGCGTGCGTGCGTGCGTGCCGTCAGCTCGCCGGGCACC
GCGGCCTCGCCCTCGCCCTCCGCCCCTGCGCCTGCACCGCGTAGA
CCGACCCCCCCCCAGCGCGCCCACCCGGTAGAGGACCCCCGCCCG
TGCCCCGACCGGTCCCCGCCTTTTTGTAAAACTTAAAGCGGGCGC
AGCATTAACGCTTCCCGCCCCGGTGACCTCTCAGGGGTCTCCCCG
CCAAAGGTGCTCCGCCGCTAAGGAACATGGCGAAGGTGGAGCAGG
TCCTGAGCCTCGAGCCGCAGCACGAGCTCAAATTCCGAGGTCCCT
TCACCGATGTTGTCACCACCAACCTAAAGCTTGGCAACCCGACAG
ACCGAAATGTGTGTTTTAAGGTGAAGACTACAGCACCACGTAGGT
ACTGTGTGAGGCCCAACAGCGGAATCATCGATGCAGGGGCCTCAA
TTAATGTATCTGTGATGTTACAGCCTTTCGATTATGATCCCAATG
AGAAAAGTAAACACAAGTTTATGGTTCAGTCTATGTTTGCTCCAA
CTGACACTTCAGATATGGAAGCAGTATGGAAGGAGGCAAAACCGG
AAGACCTTATGGATTCAAAACTTAGATGTGTGTTTGAATTGCCAG
CAGAGAATGATAAACCACATGATGTAGAAATAAATAAAATTATAT
CCACAACTGCATCAAAGACAGAAACACCAATAGTGTCTAAGTCTC
TGAGTTCTTCTTTGGATGACACCGAAGTTAAGAAGGTTATGGAAG
AATGTAAGAGGCTGCAAGGTGAAGTTCAGAGGCTACGGGAGGAGA
ACAAGCAGTTCAAGGAAGAAGATGGACTGCGGATGAGGAAGACAG
TGCAGAGCAACAGCCCCATTTCAGCATTAGCCCCAACTGGGAAGG
AAGAAGGCCTTAGCACCCGGCTCTTGGCTCTGGTGGTTTTGTTCT
TTATCGTTGGTGTAATTATTGGGAAGATTGCCTTGTAGAGGTAGC
ATGCACAGGATGGTAAATTGGATTGGTGGATCCACCATATCATGG
GATTTAAATTTATCATAACCATGTGTAAAAAGAAATTAATGTATG
ATGACATCTCACAGGTCTTGCCTTTAAATTACCCCTCCCTGCACA
CACATACACAGATACACACACACAAATATAATGTAACGATCTTTT
AGAAAGTTAAAAATGTATAGTAACTGATTGAGGGGGAAAAGAATG
ATCTTTATTAATGACAAGGGAAACCATGAGTAATGCCACAATGGC
ATATTGTAAATGTCATTTTAAACATTGGTAGGCCTTGGTACATGA
TGCTGGATTACCTCTCTTAAAATGACACCCTTCCTCGCCTGTTGG
TGCTGGCCCTTGGGGAGCTGGAGCCCAGCATGCTGGGGAGTGCGG
TCAGCTCCACACAGTAGTCCCCACGTGGCCCACTCCCGGCCCAGG
CTGCTTTCCGTGTCTTCAGTTCTGTCCAAGCCATCAGCTCCTTGG
GACTGATGAACAGAGTCAGAAGCCCAAAGGAATTGCACTGTGGCA
GCATCAGACGTACTCGTCATAAGTGAGAGGCGTGTGTTGACTGAT
TGACCCAGCGCTTTGGAAATAAATGGCAGTGCTTTGTTCACTTAA
AGGGACCAAGCTAAATTTGTATTGGTTCATGTAGTGAAGTCAAAC
TGTTATTCAGAGATGTTTAATGCATATTTAACTTATTTAATGTAT
TTCATCTCATGTTTTCTTATTGTCACAAGAGTACAGTTAATGCTG
CGTGCTGCTGAACTCTGTTGGGTGAACTGGTATTGCTGCTGGAGG
GCTGTGGGCTCCTCTGTCTCTGGAGAGTCTGGTCATGTGGAGGTG
GGGTTTATTGGGATGCTGGAGAAGAGCTGCCAGGAAGTGTTTTTT
CTGGGTCAGTAAATAACAACTGTCATAGGGAGGGAAATTCTCAGT
AGTGACAGTCAACTCTAGGTTACCTTTTTTAATGAAGAGTAGTCA
GTCTTCTAGATTGTTCTTATACCACCTCTCAACCATTACTCACAC
TTCCAGCGCCCAGGTCCAAGTCTGAGCCTGACCTCCCCTTGGGGA
CCTAGCCTGGAGTCAGGACAAATGGATCGGGCTGCAGAGGGTTAG
AAGCGAGGGCACCAGCAGTTGTGGGTGGGGAGCAAGGGAAGAGAG
AAACTCTTCAGCGAATCCTTCTAGTACTAGTTGAGAGTTTGACTG
TGAATTAATTTTATGCCATAAAAGACCAACCCAGTTCTGTTTGAC
TATGTAGCATCTTGAAAAGAAAAATTATAATAAAGCCCCAAAATT
AAGAATTCTTTTGTCATTTTGTCACATTTGCTCTATGGGGGGAAT
TATTATTTTATCATTTTTATTATTTTGCCATTGGAAGGTTAACTT
TAAAATGAGCCCTATCACTGAGAAATACGTGTTTCATGATTTAAC
TCTGTGTGTGTGTGTGTGTGTGTGTGTGTGTGTGTGTGTATTTTT
TTTTTGGTTGTCTTCAGCTGACAGTATGAAAAATGAAACTGCTGA
AAAAGCTGAGCACCTGGTCACCCTTGGCCTTCCATTGCTTTGGCC
TTCAGTAAAAAGCAGCCTCCCTTCTAGGTCAGGGAACCATGCCAT
TGAGACTAGTAACGGGCGTTCTGGGCACAGTCCCACTGTGCACAG
GTTTGAGAGGACAAGTTCATCAGAAGGAAGGCAGTCCTTAGAAGT
CACATACGTTGAGCCACGTTGCTCCTAAGCCTGGCTCTGTCAAGC
TGGGTCAGGGGCCTTGAAACTGGAGAAGTGGAAGTCTATGGTTGG
TCTGAGTAAGTAACTTCCTGTCTTCATGAAAAAAGTTGACTTTGA
ATCCCAGGTACTCACAGAAATGGTGAACAGACTTAGTTGTTACCC
AGGCACCCATGGATTGTGTTGAGTGTGCAGACAGGGAGGCCACCC
CAATAGGAATTCGTCTCCAGGATTTTTCCCATGTGTCCCCCAGTA
CTTATAAAAGGGAGTGAAAAGACCGAGCTGTAAGGCATGTGCCTT
CTGCCACCTGACTTTCCGTGAGGGGACTAAAATTTACTAATTGTA
GTTGCTGCAGCCAGTTAAGTCCTGTAGCTTCCAGGCCCTCATGTC
TTTGATAGGAGAGTGCTTAGGTGGTCCCCAACAGTGCCTAGGGGT
ACAGTACAGTCCCATTACACTAGAGCAGGGCTCTATTTATTTTTA
AAGGATATGGCCGTGTGTTTTGATAAAACTTTATTCACAAAAACA
GCCGGGTCATGGGATTTGGCTTGTGAGTCTGTAACAGTTCTTAAA
AAGAATATCTGAGAAACTACTCTGTTTTAGACCTTTGAAGGTGAT
TTAGAGTTTTGTGTACATCTAGGAGAAGGTGTTCAGCTTCTCAGA
GGATGTGGACATTTTGGTTGCAGCTAAAAATCAGTCTCTGAAGTC
TCTCTCCCTTCTAGAGGTTAGGACTTGGTGAACATGTTTGTGGGC
CTTTTGACTGAGTGGCAGAAGGAAACTGCTCAGGAAGAGAAACAG
GTGACTGATGGGAAGGTTGATTATTTTCTCAGTCATCCTGGCAGC
CAAAAATGTGCCAGGAAAAGAAAGAATGTGGAGCACGCGTGGCTC
CTGGAGGACTTGGAGATGCATGCACATTTAGGGTGTTTTCCCTAG
AATTACATAATGAAAAAAAGAATAAGGCAAAGAGGAGGTGAATAT
GGGGCCTGTCACAACGGCCTGCCCTGCCCCAAGAGGGTTAAGAGT
CAGATAATCGGGACGAAACTGGCATGGAAAGAGCGAGCCTAGGGA
GGATGCCGCTGGGCAGTGTGCATGGGGGAGCTGCTGCCAGGCTGC
CCTCCAGTCTGCTCCTGTGGTTACTGGCTCCACAGCACCTCAGAG
AGGGCGGCCCTGGCTTCAGAAATGCCAGCCATAGTGCTCACAAAT
GCAGAAGAGATGGAAGCGGTGACAGAATCCTGAAAGTTTTTATTG
ATTGAAGTTTTAAATTGGTAACTTAAGCTTCCTTGGCACGATACA
AAATACCTCTTAAAGACAGCAGGCTTTTTTATTTGTAGGTGTGAG
GAACTGGCTTTAACTTTTTTCTCCTCCTAGTTTGCATGTTTTCCT
TCTCTCGTCTTCTGAACTGCTGGCACCAGCAGTAATACATACTGA
TAAAATCAAAATTGATTTTTACCAGTGGCCAGTTTATGGCTAGAG
AGACGACTTATACCTCCATAACACAGAAGGGGGAAAAATGAAGAA
CCTCCAGTGATCCGTGAAAACCTAAACGCTTTCAAACAAATCCCA
GGAACAGAATTGCTATCGAAAGATATCATTGCCCAGTTTGCAGGC
TATGTTGAGTCAGATAGAACTGAATGTAGTGAGAGCTCAGAGCTA
CAGAGCCTTTCAGATGAATTTGAAAACAGACTCTGTGTGTGTGTG
CATGTGTGCATGTGTGCATGTGTGGCATATGTGCCGTATGTCAGT
AGCTTGACAGTTTTCAAATCGTGCCTATATTTTTTTGCATACACA
AATTTTTGTGTTTGCAAACTCAGAATCCATGCCAAAATACAATGT
TATATGTCATTTTCAGCTCCTTCTCTAAAGGAATGGCCCATTTCT
CATTGTAGTTTGAGAAATACATGTATGAAGAGATAGGGGTCTTGG
GCTTCCCAGTGTCACTTTGAACACCTGAATAACATTTAACTCCTG
AGACCTTCTCGGTGTAGAGGCCACTGCTTCCCCCTGCTGGAGATG
GCATTTCATTGAAGGGCCTCTCGTGGCTTTCCCTGCCCCCGGCTG
TCTGGCCTGAAGAAGGAGAAAGAACCAAACTGAACTATGAAAAGT
TACCACTCTGAGGAGACCTCTCTTAATTAACACTTGGGGCCATGT
TTGCTGTTGTTGAGAAGGAGTGTTCTCAAAGATGAGCTGGAATGG
AATTGTATTTAGAAAGGCCCCTGCAAAGTATATAGATGGATGACT
CTAGTTCATGACATACAAATCCCATAAGGCCAACGACCACTCTTC
TGGAACACCAAGAGCAGCTCTGAGATCATGCTGGCCCTACGCGAA
TTGAGTTTCTGTGGCCTAATTGGATTTGGAGAACGCCTTCCCTGG
CCCCTTTTCCTCAGACAGATCTGCTCTGATAGGAACCTTTTCAAG
AAAGTTACTGTTGTTTCAATGCCACTCCTTACCTGTATAGAACAT
TTCCAATACATTCGCTCATTGAACTTAATCCTTGCAACTGTGACT
GGGGGGTAGATGGCTCTGTTTGCATACGAAGAAATAAAGGCTCCA
GGAGGTTAAATCGGGCAACTTTTTAGAACTAAATCAGTCTCTGTA
AGGCCTACATTGCTAAGATACCATTTCAGCTCTGAAAATCTGCTT
CAGGGAAGTGAGTGGATGAGGCCTTCCTGCCTCAGCTACTCTGCC
CGTCTGTACATCTTTTGTGTCTGCCTCCGTACCTTATTCAGTTAT
TTTCACACTAAAGTAAGTAGAATTAAGACTGTAGTTCAGATGCTT
TTTCTTTTTCTGTTGGAAACTGAACACACTACAGACAGTGAAAAA
AGGTACATATTCCATTTTCTCATTGCCTGAAGATCTCTGCTGATG
CTCCTGGAGAATGACTTTGGGGGCTTTAGAAAGAATATTGCCAGT
CCGTCTCGGCAAGGAGATGATGGGAGCGCTTTATATGGAGGCTTT
ACATGACTTGTAAATTAAATGTGAATGAGGGCAGTTGATTAAAAT
TGGTATTACAGAAGGGCCCTGCTGAGGTTTGAAAACAGCTGAGCT
GCTGATGTCTCAGGCCTTTCCCTGAATTAGCACTGCGGTTCTCCA
GGATATCAGCAAAGAGGGCAAGTAATAGAAGCCCCTGATAAGGAG
CGTCAGCCGACAGGCAAGCTTGGGAGGCTGTGGGAATGGGTCTGC
CCCCAGCTTCACAGACCTCTTCCTCCAGCCTCTGAATCCCATTAG
CCACAGCCTAGAACATTAGCTGAGCTGCACAAGCTCACCCACCCC
TGTGCCAGGGGGCCCTGACCTCCCTCCATGCCATGTTTTTGGCTG
TATCTACGGCACTTAACAATAGGGGCTTTTTATTTTCATTACAGA
GATATTTTGAAAAATTTAAAAGACATGAACTCACATAAACAGTTA
TGGATGATAGTTAAAAGAGAAACGGGTGGAGGTGGATGAGAGGTT
GTCTTCATGAATATAATTACTTGAGATTTTTTTTTCTTAATGGAA
TTAGTTTATTAGAAAATGTCTGTGTTAAATCCGTAGAAAAGGAAG
AAAAGTGTAGCAACAAAAATGTAGCCATTATCTAACTTGCCATAA
ATATTTGCAGTTATGATACCTTGGAATGTTGCCACGATATGGATT
GCTTTGATTAAAAGATGTCAGTTGAATAAAACAGTACTGTGGGAG
AATCGCTTTCTGCTGCTAGATAAATGCTGATGTTTATTTTTAAAC
CAGGAAACATTGATCCTGTAACAATGCCCGATTACAATTGCTTTA
TTACACCCCAGGGCTGATGGAGATGTAATCACTTGGCTAATGGAT
GTGGGTGCAGGACAGATGCTCGCTTGCTGGCCTGCTTTCCTGCTT
GCATTCTGATGAGCTGCAGGAGTGCGCCTGGCCTTCTGCAGGTGG
AGCTGCTGTCAGAGCTTCGTTTCACTGATACCCAAAGCCATGTCT
GACTGAAATAAAACAGGTTCCCTTTTTTTTTCCCTTTGGAAAATG
CCAACTAAGGGAGACTAATCAGATATCTTAACACAATTTCATCCA
GGCTTAGTGCTAACAAGATTGCGGGGCTTTTTAGGGTTTAAGAAG
ATGAGAAATGAGTGTGCACGTTTCACACGTTGACTTGCCGGTTTT
TCCATGTCATACAAAAAAGTCCTGGCTGTTTCTCCGAACTGGCTG
CCTGCATTCCCGTCTTTCTTTTGTTTTTAAGAAATAGACTGAATT
CAGCTGTTAATCCTCTAGTACAGTATCCATGTTAAAATGTTTTTC
CATTGCATCTTTTATGTGAATTCAAAGGTCAGAATTTATTGTCTG
TGATATTGAGACCATGTGTACAAGAACTACTTTTTGCTTTTCATC
ATTCACTCCTTAGCAAACGTTTCGTAAGTACCCTCTGTCTGTTTG
CTACTATATGAGGTGCTGCGAAATTAGTGGGCGTGGCTTTTTATA
TTTTTCATTCGTGTGTAGCCTAAGTAAGGTGACTCAAGATGATAC
ACCGAGAGAAAAATGCAAAATATATTTGGTTCTCATTTCTGTTGC
TGTCGTTTCCTTTTTAAAGACGATTTATCAACTGCTGCCATTTGG
AACTTCCTATAAGAAACTAAAAATGATCTATTTCAGTGTTCCTTT
CGCCTTTCCTCTGCTTTCTGAATAAATGGTTTCAGTAACCCATGC
TGTTCTCTCCCTATTCTACGTCTTTCTCCCTATGTTGAAAAAAGA
TTCCCACAGTTTCTGATGTGTGTGTTTATAGTCTTCAATGTATGT
TAACATGTTAGGAACTGAGTATCTTAAGAGATGTCTTAGAATGCT
TTAGTTTTCATAATTTGTCCTTTATGTATTTTTCATTGTATTTGC
TGTTTTGACATGGAAGTAATTTAAAAAGTTGGTGCAGGAAAGGAC
TCTTTACTGTTGCACATTTTGGTTTTCTGATATGTAATAAATTCA
TGGCTTGGCAGCTGACATGATGTTTCCCAGAGAGAAGGAGATGTA
TTTCTGCAGGGTCCAGACCAAAAGAGCCATTTACAGCATGTTCTC
CCATGTTCCATTATCAGCCTGATGAAACCTGCCCTGCCAAGGCAT
AAACTTTTGTACTAGCTGTCTCCATATTATGTTCAATAAATTCTG
TGCTCTGAATATATTTAAAAAAAAAAAAA

### CADM1

CADM1-NRG1 fusion sequence
SEQ ID NO: 27
AGCTTCAAACATAGTGGGGAAAGCTCACTCGGATTATATGCTGTA
TGTATACGCTACATCTACATCCACCACTGGGACAAGCCAT

CADM1 sequence information, providing nucleotide sequence of exons 1-11 below as indicated by alternating underlining, starting with exon 1 underlined, exon 2 not underlined, exon 3 underlined, exon 4 not underlined, and so forth.
SEQ ID NO:28=Exon 1 CADM1
SEQ ID NO:29=Exon 2 CADM1
SEQ ID NO:30=Exon 3 CADM1
SEQ ID NO:31=Exon 4 CADM1
SEQ ID NO:32=Exon 5 CADM1
SEQ ID NO:33=Exon 6 CADM1
SEQ ID NO:34=Exon 7 CADM1
SEQ ID NO: 35=Exon 8 CADM1
SEQ ID NO:36=Exon 9 CADM1
SEQ ID NO:37=Exon 10 CADM1
SEQ ID NO:38=Exon 11 CADM1
SEQ ID NO:39=Exons 1-11 CADM1
SEQ ID NO:40=Exons 1-7 CADM1

SEQ ID NO: 39
GGTTGGGCTCGCGGCGCTGTGATTGGTCTGCCCGGACTCCGCCTC
CAGCGCATGTCATTAGCATCTCATTAGCTGTCCGCTCGGGCTCCG
GAGGCAGCCAACGCCGCCAGTCTGAGGCAGGTGCCCGACATGGCG
AGTGTAGTGCTGCCGAGCGGATCCCAGTGTGCGGCGGCAGCGGCG
GCGGCGGCGCCTCCCGGGCTCCGGCTCCGGCTTCTGCTGTTGCTC
TTCTCCGCCGCGGCACTGATCCCCACAGGTGATGGGCAGAATCTG
TTTACGAAAGACGTGACAGTGATCGAGGGAGAGGTTGCGACCATC
AGTTGCCAAGTCAATAAGAGTGACGACTCTGTGATTCAGCTACTG
AATCCCAACAGGCAGACCATTTATTTCAGGGACTTCAGGCCTTTG
AAGGACAGCAGGTTTCAGTTGCTGAATTTTTCTAGCAGTGAACTC
AAAGTATCATTGACAAACGTCTCAATTTCTGATGAAGGAAGATAC
TTTTGCCAGCTCTATACCGATCCCCCACAGGAAAGTTACACCACC
ATCACAGTCCTGGTCCCACCACGTAATCTGATGATCGATATCCAG
AAAGACACTGCGGTGGAAGGTGAGGAGATTGAAGTCAACTGCACT
GCTATGGCCAGCAAGCCAGCCACGACTATCAGGTGGTTCAAAGGG
AACACAGAGCTAAAAGGCAAATCGGAGGTGGAAGAGTGGTCAGAC
ATGTACACTGTGACCAGTCAGCTGATGCTGAAGGTGCACAAGGAG
GACGATGGGGTCCCAGTGATCTGCCAGGTGGAGCACCCTGCGGTC
ACTGGAAACCTGCAGACCCAGCGGTATCTAGAAGTACAGTATAAG
CCTCAAGTGCACATTCAGATGACTTATCCTCTACAAGGCTTAACC
CGGGAAGGGGACGCGCTTGAGTTAACATGTGAAGCCATCGGGAAG
CCCCAGCCTGTGATGGTAACTTGGGTGAGAGTCGATGATGAAATG
CCTCAACACGCCGTACTGTCTGGGCCCAACCTGTTCATCAATAAC
CTAAACAAAACAGATAATGGTACATACCGCTGTGAAGCTTCAAAC
ATAGTGGGGAAAGCTCACTCGGATTATATGCTGTATGTATACGAC
ACAACGGCGACGACAGAACCAGCAGTTCACGGCCTTACTCAGTTG
CCCAATTCCGCAGAAGAACTGGACAGTGAGGACCTCTCAGATTCC
CGAGCAGGTGAAGAAGGCTCGATCAGGGCAGTGGATCATGCCGTG
ATCGGTGGCGTCGTGGCGGTGGTGGTGTTCGCCATGCTGTGCTTG
CTCATCATTCTGGGGCGCTATATCGGTGGCGTCGTGGCGGTGGTG
GTGTTCGCCATGCTGTGCTTGCTCATCATTCTGGGGCGCTATTTT
GCCAGACATAAAGGTACATACTTCACTCATGAAGCCAAAGGAGCC
GATGACGCAGCAGACGCAGACACAGCTATAATCAATGCAGAAGGA
GGACAGAACAACTCCGAAGAAAAGAAAGAGTACTTCATCTAGATC
AGCCTTTTTGTTTCAATGAGGTGTCCAACTGGCCCTATTTAGATG
ATAAAGAGACAGTGATATTGGAACTTGCGAGAAATTCGTGTGTTT
TTTTATGAATGGGTGGAAAGGTGTGAGACTGGGAAGGCTTGGGAT
TTGCTGTGTAAAAAAAAAAAAAATGTTCTTTGGAAAGTACACTCT
GCTGTTTGACACCTCTTTTTTCGTTTGTTTGTTTGTTTAATTTTT
ATTTCTTCCTACCAAGTCAAACTTGGATACTTGGATTTAGTTTCA
GTAGATTGCAGAAAATTCTGTGCCTTGTTTTTTGTTTGTTTGTTG
CGTTCCTTTCTTTTCCCCCTTTGTGCACATTTATTTCCTCCCTCT
ACCCCAATTTCGGATTTTTTCCAAAATCTCCCATTTTGGAATTTG
CCTGCTGGGATTCCTTAGACTCTTTTCCTTCCCTTTTCTGTTCTA
GTTTTTTACTTTTGTTTATTTTTATGGTAACTGCTTTCTGTTCCA
AATTCAGTTTCATAAAAGGAGAACCAGCACAGCTTAGATTTCATA
GTTCAGAATTTAGTGTATCCATAATGCATTCTTCTCTGTTGTCGT
AAAGATTTGGGTGAACAAACAATGAAAACTCTTTGCTGCTGCCCA
TGTTTCAAATACTTAGAGCAGTGAAGACTAGAAAATTAGACTGTG
ATTCAGAAAATGTTCTGTTTGCTGTGGAACTACATTACTGTACAG
GGTTATCTGCAAGTGAGGTGTGTCACAATGAGATTGAATTTCACT
GTCTTTAATTCTGTATCTGTAGACGGCTCAGTATAGATACCCTAC
GCTGTCCAGAAAGGTTTGGGGCAGAAAGGACTCCTCCTTTTTCCA
TGCCCTAAACAGACCTGACAGGTGAGGTCTGTTCCTTTTATATAA
GTGGACAAATTTTGAGTTGCCACAGGAGGGGAAGTAGGGAGGGGG
GAAATACAGTTCTGCTCTGGTTGTTTCTGTTCCAAATGATTCCAT
CCACCTTTCCCAATCGGCCTTACTTCTCACTAATTTGTAGGAAAA
AGCAAGTTCGTCTGTTGTGCGAATGACTGAATGGGACAGAGTTGA
TTTTTTTTTTTTTTTCCTTTGTGCTTAGTTAGGAAGGCAGTAGGA
TGTGGCCTGCATGTACTGTATATTACAGATATTTGTCATGCTGGG
ATTTCCAACTCGAATCTGTGTGAAACTTTCATTCCTTCAGATTTG
GCTTGACAAAGGCAGGAGGTACAAAAGAAGGGCTGGTATTGTTCT
CACACTGGTCTGCTGTCGCTCTCAGTTCTCGATAGGTCAGAGCAG
AGGTGGAAAAACAGCATGTACGGATTTTCAGTTACTTAATCAAAA
CTCAAATGTGAGTGTTTTTATCTTTTTACCTTTCATACACTAGCC
TTGGCCTCTTTCCTCAGCCTTAAGAACCATCTGCCAAAAATTACT
GATCCTCGCATGATGGCAGCCATAGTGCATAGCTACTAAAATCAG
TGACCTTGAACATATCTTAGATGGGGAGCCTCGGGAAAAGGTAGA
GGAGTCACGTTACCATTTACATGTTTTAAAGAAAGAAGTGTGGGG
ATTTTCACTGAAACGTCTAGGAAATCTAGAAGTAGTCCTGAAGGA
CAGAAACTAAACTCTTACCATATGTTTGGTAAGACTCCAGACTCC
AGCTAACAGTCCCTATGGAAAGATGGCATCAAAAAAGATAGATCT
ATATATATATATAAATATATATTCTATTACATTTTCAGTGAGTAA
TTTTGGATTTTGCAAGGTGCATTTTTACTATTGTTACATTATGTG
GAAAACTTATGCTGATTTATTTAAGGGGGAAAAAGTGTCAACTCT
TTGTTATTTGAAAACATGTTTATTTTTCTTGTCTTTATTTTAACC
TTTGATAGAACCATTGCAATATGGGGGCCTTTTGGGAACGGACTG
GTATGTAAAAGAAAATCCATTATCGAGCAGCATTTTATTTACCCC
TCCCCTATCCCTAGGCACTTAACCAAGACAAAAAGCCACAATGAA
CATCCCTTTTTCAATGAATTTTATAATCTGCAGCTCTATTCCGAG
CCCTTAGCACCCATTCCGACCATAGTATAATCATATCAAAGGGTG
AGAATCATTTAGCATGTTGTTGAAAGGTTTTTTTTCAGTTGTTCT
TTTTAGAAAAAAAGAAAAACAAAAACAAAAACAAAAAAAAAAAAT
CACACCATTGCTCACAGAATTGGCATCTCATTTTTGGGACCTCCC
ATCTTTCTGTTTTGAAAAGTGTACAGTAGTGCAGTGTTCCTGATG
TAACTTTATGGCTTACAATGTTGACATGTCTCAGGTTCATGTGTT
GCGATTGGTGTTTTCCGTCTCAGGTAGATTGCAAAGTGTAGGCCC
CACACATTGGAAAAAATAATAATAAAACAAAGCAAAAACAGGAAA
TTATGGATTTTAGTTGTATATTGGTTTATGTATTTTTTCTTAAGT
ATACAGTGCACTGTTTGAAATGTATTGTTGAGTATTACTTTGTAC
AGGTTGATCACTTTTTTTAGAGTGAAGAAAGAACAAACTTGTTTT
TTGTGTTTTTTAAAGGAATATAAAATAATGAAGGATGTATAATTG
ATGCCAAATAAGCTTGTTCTTTAGTCACACCGACGTCTTATTTTT
CCCTTTAGGCCAGTTCTGTTTTTAAGGTGTACATGGACAATGTTA
CAGTGTAAGAAACTCCATATCCATATGTTCCCATTCGCATTTTGT
ATTGGTTCATGTATACCATTTTTACAAAAAAAAAAAGAAAAAAAA
GAAGTACTATAAAATATCTGTCTTCTTAATAAAAAAAAATTAATG
TTACAAAGTGA

### CD44

CD44-NRG1 fusion sequence
SEQ ID NO: 41
GACGAAGACAGTCCCTGGATCACCGACAGCACAGACAGAATCCCT
GCTACCACCTTGCCTCCCCGATTGAAAGAGATGAAAAGCCAGGAA
TCGGCTGCAGGTTCCAAACT

CD44 sequence information, providing nucleotide sequence of exons 1-18 below as indicated by alternating underlining, starting with exon 1 underlined, exon 2 not underlined, exon 3 underlined, exon 4 not underlined, and so forth.
SEQ ID NO:42=Exon 1 CD44
SEQ ID NO:43=Exon 2 CD44
SEQ ID NO:44=Exon 3 CD44
SEQ ID NO:45=Exon 4 CD44
SEQ ID NO:46=Exon 5 CD44
SEQ ID NO:47=Exon 6 CD44
SEQ ID NO:48=Exon 7 CD44
SEQ ID NO:49=Exon 8 CD44
SEQ ID NO:50=Exon 9 CD44
SEQ ID NO:51=Exon 10 CD44
SEQ ID NO:52=Exon 11 CD44
SEQ ID NO:53=Exon 12 CD44
SEQ ID NO:54=Exon 13 CD44
SEQ ID NO:55=Exon 14 CD44
SEQ ID NO:56=Exon 15 CD44
SEQ ID NO:57=Exon 16 CD44
SEQ ID NO:58=Exon 17 CD44
SEQ ID NO:59=Exon 18 CD44
SEQ ID NO:60=Exons 1-18 CD44
SEQ ID NO:61=Exons 1-5 CD44

SEQ ID NO: 60
CTCATTGCCCAGCGGACCCCAGCCTCTGCCAGGTTCGGTCCGCCA
TCCTCGTCCCGTCCTCCGCCGGCCCCTGCCCCGCGCCCAGGGATC
CTCCAGCTCCTTTCGCCCGCGCCCTCCGTTCGCTCCGGACACCAT
GGACAAGTTTTGGTGGCACGCAGCCTGGGGACTCTGCCTCGTGCC
GCTGAGCCTGGCGCAGATCGATTTGAATATAACCTGCCGCTTTGC
AGGTGTATTCCACGTGGAGAAAAATGGTCGCTACAGCATCTCTCG
GACGGAGGCCGCTGACCTCTGCAAGGCTTTCAATAGCACCTTGCC
CACAATGGCCCAGATGGAGAAAGCTCTGAGCATCGGATTTGAGAC
CTGCAGGTATGGGTTCATAGAAGGGCACGTGGTGATTCCCCGGAT
CCACCCCAACTCCATCTGTGCAGCAAACAACACAGGGGTGTACAT
CCTCACATCCAACACCTCCCAGTATGACACATATTGCTTCAATGC
TTCAGCTCCACCTGAAGAAGATTGTACATCAGTCACAGACCTGCC
CAATGCCTTTGATGGACCAATTACCATAACTATTGTTAACCGTGA
TGGCACCCGCTATGTCCAGAAAGGAGAATACAGAACGAATCCTGA
AGACATCTACCCCAGCAACCCTACTGATGATGACGTGAGCAGCGG
CTCCTCCAGTGAAAGGAGCAGCACTTCAGGAGGTTACATCTTTTA
CACCTTTTCTACTGTACACCCCATCCCAGACGAAGACAGTCCCTG
GATCACCGACAGCACAGACAGAATCCCTGCTACCACTTTGATGAG
CACTAGTGCTACAGCAACTGAGACAGCAACCAAGAGGCAAGAAAC
CTGGGATTGGTTTTCATGGTTGTTTCTACCATCAGAGTCAAAGAA
TCATCTTCACACAACAACACAAATGGCTGGTACGTCTTCAAATAC
CATCTCAGCAGGCTGGGAGCCAAATGAAGAAAATGAAGATGAAAG
AGACAGACACCTCAGTTTTTCTGGATCAGGCATTGATGATGATGA
AGATTTTATCTCCAGCACCATTTCAACCACACCACGGGCTTTTGA
CCACACAAAACAGAACCAGGACTGGACCCAGTGGAACCCAAGCCA
TTCAAATCCGGAAGTGCTACTTCAGACAACCACAAGGATGACTGA
TGTAGACAGAAATGGCACCACTGCTTATGAAGGAAACTGGAACCC
AGAAGCACACCCTCCCCTCATTCACCATGAGCATCATGAGGAAGA
AGAGACCCCACATTCTACAAGCACAATCCAGGCAACTCCTAGTAG
TACAACGGAAGAAACAGCTACCCAGAAGGAACAGTGGTTTGGCAA
CAGATGGCATGAGGGATATCGCCAAACACCCAAAGAAGACTCCCA
TTCGACAACAGGGACAGCTGCAGCCTCAGCTCATACCAGCCATCC
AATGCAAGGAAGGACAACACCAAGCCCAGAGGACAGTTCCTGGAC
TGATTTCTTCAACCCAATCTCACACCCCATGGGACGAGGTCATCA
AGCAGGAAGAAGGATGGATATGGACTCCAGTCATAGTATAACGCT
TCAGCCTACTGCAAATCCAAACACAGGTTTGGTGGAAGATTTGGA
CAGGACAGGACCTCTTTCAATGACAACGCAGCAGAGTAATTCTCA
GAGCTTCTCTACATCACATGAAGGCTTGGAAGAAGATAAAGACCA
TCCAACAACTTCTACTCTGACATCAAGCAATAGGAATGATGTCAC
AGGTGGAAGAAGAGACCCAAATCATTCTGAAGGCTCAACTACTTT
ACTGGAAGGTTATACCTCTCATTACCCACACACGAAGGAAAGCAG
GACCTTCATCCCAGTGACCTCAGCTAAGACTGGGTCCTTTGGAGT
TACTGCAGTTACTGTTGGAGATTCCAACTCTAATGTCAATCGTTC
CTTATCAGGAGACCAAGACACATTCCACCCCAGTGGGGGGTCCCA
TACCACTCATGGATCTGAATCAGATGGACACTCACATGGGAGTCA
AGAAGGTGGAGCAAACACAACCTCTGGTCCTATAAGGACACCCCA
AATTCCAGAATGGCTGATCATCTTGGCATCCCTCTTGGCCTTGGC
TTTGATTCTTGCAGTTTGCATTGCAGTCAACAGTCGAAGAAGGTG
TGGGCAGAAGAAAAAGCTAGTGATCAACAGTGGCAATGGAGCTGT
GGAGGACAGAAAGCCAAGTGGACTCAACGGAGAGGCCAGCAAGTC
TCAGGAAATGGTGCATTTGGTGAACAAGGAGTCGTCAGAAACTCC
AGACCAGTTTATGACAGCTGATGAGACAAGGAACCTGCAGAATGT
GGACATGAAGATTGGGGTGTAACACCTACACCATTATCTTGGAAA
GAAACAACCGTTGGAAACATAACCATTACAGGGAGCTGGGACACT
TAACAGATGCAATGTGCTACTGATTGTTTCATTGCGAATCTTTTT
TAGCATAAAATTTTCTACTCTTTTTGTTTTTTGTGTTTTGTTCTT
TAAAGTCAGGTCCAATTTGTAAAAACAGCATTGCTTTCTGAAATT
AGGGCCCAATTAATAATCAGCAAGAATTTGATCGTTCCAGTTCCC
ACTTGGAGGCCTTTCATCCCTCGGGTGTGCTATGGATGGCTTCTA
ACAAAAACTACACATATGTATTCCTGATCGCCAACCTTTCCCCCA
CCAGCTAAGGACATTTCCCAGGGTTAATAGGGCCTGGTCCCTGGG
AGGAAATTTGAATGGGTCCATTTTGCCCTTCCATAGCCTAATCCC
TGGGCATTGCTTTCCACTGAGGTTGGGGGTTGGGGTGTACTAGTT
ACACATCTTCAACAGACCCCCTCTAGAAATTTTTCAGATGCTTCT
GGGAGACACCCAAAGGGTGAAGCTATTTATCTGTAGTAAACTATT
TATCTGTGTTTTTGAAATATTAAACCCTGGATCAGTCCTTTGATC
AGTATAATTTTTTAAAGTTACTTTGTCAGAGGCACAAAAGGGTTT
AAACTGATTCATAATAAATATCTGTACTTCTTCGATCTTCACCTT
TTGTGCTGTGATTCTTCAGTTTCTAAACCAGCACTGTCTGGGTCC
CTACAATGTATCAGGAAGAGCTGAGAATGGTAAGGAGACTCTTCT
AAGTCTTCATCTCAGAGACCCTGAGTTCCCACTCAGACCCACTCA
GCCAAATCTCATGGAAGACCAAGGAGGGCAGCACTGTTTTTGTTT
TTTGTTTTTTGTTTTTTTTTTTTGACACTGTCCAAAGGTTTTCCA
TCCTGTCCTGGAATCAGAGTTGGAAGCTGAGGAGCTTCAGCCTCT
TTTATGGTTTAATGGCCACCTGTTCTCTCCTGTGAAAGGCTTTGC
AAAGTCACATTAAGTTTGCATGACCTGTTATCCCTGGGGCCCTAT
TTCATAGAGGCTGGCCCTATTAGTGATTTCCAAAAACAATATGGA
AGTGCCTTTTGATGTCTTACAATAAGAGAAGAAGCCAATGGAAAT
GAAAGAGATTGGCAAAGGGGAAGGATGATGCCATGTAGATCCTGT
TTGACATTTTTATGGCTGTATTTGTAAACTTAAACACACCAGTGT
CTGTTCTTGATGCAGTTGCTATTTAGGATGAGTTAAGTGCCTGGG
GAGTCCCTCAAAAGGTTAAAGGGATTCCCATCATTGGAATCTTAT
CACCAGATAGGCAAGTTTATGACCAAACAAGAGAGTACTGGCTTT
ATCCTCTAACCTCATATTTTCTCCCACTTGGCAAGTCCTTTGTGG
CATTTATTCATCAGTCAGGGTGTCCGATTGGTCCTAGAACTTCCA
AAGGCTGCTTGTCATAGAAGCCATTGCATCTATAAAGCAACGGCT
CCTGTTAAATGGTATCTCCTTTCTGAGGCTCCTACTAAAAGTCAT
TTGTTACCTAAACTTATGTGCTTAACAGGCAATGCTTCTCAGACC
ACAAAGCAGAAAGAAGAAGAAAAGCTCCTGACTAAATCAGGGCTG
GGCTTAGACAGAGTTGATCTGTAGAATATCTTTAAAGGAGAGATG
TCAACTTTCTGCACTATTCCCAGCCTCTGCTCCTCCCTGTCTACC
CTCTCCCCTCCCTCTCTCCCTCCACTTCACCCCACAATCTTGAAA
AACTTCCTTTCTCTTCTGTGAACATCATTGGCCAGATCCATTTTC
AGTGGTCTGGATTTCTTTTTATTTTCTTTTCAACTTGAAAGAAAC
TGGACATTAGGCCACTATGTGTTGTTACTGCCACTAGTGTTCAAG
TGCCTCTTGTTTTCCCAGAGATTTCCTGGGTCTGCCAGAGGCCCA
GACAGGCTCACTCAAGCTCTTTAACTGAAAAGCAACAAGCCACTC
CAGGACAAGGTTCAAAATGGTTACAACAGCCTCTACCTGTCGCCC
CAGGGAGAAAGGGGTAGTGATACAAGTCTCATAGCCAGAGATGGT
TTTCCACTCCTTCTAGATATTCCCAAAAAGAGGCTGAGACAGGAG
GTTATTTTCAATTTTATTTTGGAATTAAATACTTTTTTCCCTTTA
TTACTGTTGTAGTCCCTCACTTGGATATACCTCTGTTTTCACGAT
AGAAATAAGGGAGGTCTAGAGCTTCTATTCCTTGGCCATTGTCAA
CGGAGAGCTGGCCAAGTCTTCACAAACCCTTGCAACATTGCCTGA
AGTTTATGGAATAAGATGTATTCTCACTCCCTTGATCTCAAGGGC
GTAACTCTGGAAGCACAGCTTGACTACACGTCATTTTTACCAATG
ATTTTCAGGTGACCTGGGCTAAGTCATTTAAACTGGGTCTTTATA
AAAGTAAAAGGCCAACATTTAATTATTTTGCAAAGCAACCTAAGA
GCTAAAGATGTAATTTTTCTTGCAATTGTAAATCTTTTGTGTCTC
CTGAAGACTTCCCTTAAAATTAGCTCTGAGTGAAAAATCAAAAGA
GACAAAAGACATCTTCGAATCCATATTTCAAGCCTGGTAGAATTG
GCTTTTCTAGCAGAACCTTTCCAAAAGTTTTATATTGAGATTCAT
AACAACACCAAGAATTGATTTTGTAGCCAACATTCATTCAATACT
GTTATATCAGAGGAGTAGGAGAGAGGAAACATTTGACTTATCTGG
AAAAGCAAAATGTACTTAAGAATAAGAATAACATGGTCCATTCAC
CTTTATGTTATAGATATGTCTTTGTGTAAATCATTTGTTTTGAGT
TTTCAAAGAATAGCCCATTGTTCATTCTTGTGCTGTACAATGACC
ACTGTTATTGTTACTTTGACTTTTCAGAGCACACCCTTCCTCTGG
TTTTTGTATATTTATTGATGGATCAATAATAATGAGGAAAGCATG
ATATGTATATTGCTGAGTTGAAAGCACTTATTGGAAAATATTAAA
AGGCTAACATTAAAAGACTAAAGGAAACAGA

### SLC3A2

SLC3A2-NRG1 fusion sequence
SEQ ID NO: 62
CCGCATCGGCGACCTTCAGGCCTTCCAGGGCCACGGCGCGGGCAA
CCTGGCGGCATCTACATCTACATCCACCACTGGGACAAGCCAT

SLC3A2 transcript version 6 sequence information, providing nucleotide sequence of exons 1-9 below as indicated by alternating underlining, starting with exon 1 underlined, exon 2 not underlined, exon 3 underlined, exon 4 not underlined, and so forth.
SEQ ID NO:63=Exon 1 SLC3A2 transcript version 6
SEQ ID NO:64=Exon 2 SLC3A2 transcript version 6
SEQ ID NO:65=Exon 3 SLC3A2 transcript version 6
SEQ ID NO:66=Exon 4 SLC3A2 transcript version 6
SEQ ID NO:67=Exon 5 SLC3A2 transcript version 6
SEQ ID NO:68=Exon 6 SLC3A2 transcript version 6
SEQ ID NO:69=Exon 7 SLC3A2 transcript version 6
SEQ ID NO:70=Exon 8 SLC3A2 transcript version 6
SEQ ID NO:71=Exon 9 SLC3A2 transcript version 6
SEQ ID NO:72=Exons 1-9 SLC3A2 transcript version 6
SEQ ID NO:73=Exon 2-9 SLC3A2 transcript version 6

SEQ ID NO: 72
AGATGCAGTAGCCGAAACTGCGCGGAGGCACAGAGGCCGGGGAGA
GCGTTCTGGGTCCGAGGGTCCAGGTAGGGGTTGAGCCACCATCTG
ACCGCAAGCTGCGTCGTGTCGCCGGTTCTGCAGGCACCATGAGCC
AGGACACCGAGGTGGATATGAAGGAGGTGGAGCTGAATGAGTTAG
AGCCCGAGAAGCAGCCGATGAACGCGGCGTCTGGGGCGGCCATGT
CCCTGGCGGGAGCCGAGAAGAATGGTCTGGTGAAGATCAAGGTGG
CGGAAGACGAGGCGGAGGCGGCAGCCGCGGCTAAGTTCACGGGCC
TGTCCAAGGAGGAGCTGCTGAAGGTGGCAGGCAGCCCCGGCTGGG
TACGCACCCGCTGGGCACTGCTGCTGCTCTTCTGGCTCGGCTGGC
TCGGCATGCTTGCTGGTGCCGTGGTCATAATCGTGCGAGCGCCGC
GTTGTCGCGAGCTACCGGCGCAGAAGTGGTGGCACACGGGCGCCC
TCTACCGCATCGGCGACCTTCAGGCCTTCCAGGGCCACGGCGCGG
GCAACCTGGCGGGTCTGAAGGGGCGTCTCGATTACCTGAGCTCTC
TGAAGGTGAAGGGCCTTGTGCTGGGTCCAATTCACAAGAACCAGA
AGGATGATGTCGCTCAGACTGACTTGCTGCAGATCGACCCCAATT
TTGGCTCCAAGGAAGATTTTGACAGTCTCTTGCAATCGGCTAAAA
AAAAGAGCATCCGTGTCATTCTGGACCTTACTCCCAACTACCGGG
GTGAGAACTCGTGGTTCTCCACTCAGGTTGACACTGTGGCCACCA
AGGTGAAGGATGCTCTGGAGTTTTGGCTGCAAGCTGGCGTGGATG
GGTTCCAGGTTCGGGACATAGAGAATCTGAAGGATGCATCCTCAT
TCTTGGCTGAGTGGCAAAATATCACCAAGGGCTTCAGTGAAGACA
GGCTCTTGATTGCGGGGACTAACTCCTCCGACCTTCAGCAGATCC
TGAGCCTACTCGAATCCAACAAAGACTTGCTGTTGACTAGCTCAT
ACCTGTCTGATTCTGGTTCTACTGGGGAGCATACAAAATCCCTAG
TCACACAGTATTTGAATGCCACTGGCAATCGCTGGTGCAGCTGGA
GTTTGTCTCAGGCAAGGCTCCTGACTTCCTTCTTGCCGGCTCAAC
TTCTCCGACTCTACCAGCTGATGCTCTTCACCCTGCCAGGGACCC
CTGTTTTCAGCTACGGGGATGAGATTGGCCTGGATGCAGCTGCCC
TTCCTGGACAGCCTATGGAGGCTCCAGTCATGCTGTGGGATGAGT
CCAGCTTCCCTGACATCCCAGGGGCTGTAAGTGCCAACATGACTG
TGAAGGGCCAGAGTGAAGACCCTGGCTCCCTCCTTTCCTTGTTCC
GGCGGCTGAGTGACCAGCGGAGTAAGGAGCGCTCCCTACTGCATG
GGGACTTCCACGCGTTCTCCGCTGGGCCTGGACTCTTCTCCTATA
TCCGCCACTGGGACCAGAATGAGCGTTTTCTGGTAGTGCTTAACT
TTGGGGATGTGGGCCTCTCGGCTGGACTGCAGGCCTCCGACCTGC
CTGCCAGCGCCAGCCTGCCAGCCAAGGCTGACCTCCTGCTCAGCA
CCCAGCCAGGCCGTGAGGAGGGCTCCCCTCTTGAGCTGGAACGCC
TGAAACTGGAGCCTCACGAAGGGCTGCTGCTCCGCTTCCCCTACG
CGGCCTGACTTCAGCCTGACATGGACCCACTACCCTTCTCCTTTC
CTTCCCAGGCCCTTTGGCTTCTGATTTTTCTCTTTTTTAAAAACA
AACAAACAAACTGTTGCAGATTATGAGTGAACCCCCAAATAGGGT
GTTTTCTGCCTTCAAATAAAAGTCACCCCTGCATGGTGAA

### VTCN1

SEQ ID NO 74: VTCN1-NRG1 fusion sequence
CATAATTAGCATCATCATTATTCTGGCTGGAGCAATTGCACTCAT
CATTGGCTTTGGTATTTCAGCCTTGCCTCCCCGATTGAAAGAGAT
GAA

VTCN1 sequence information, providing nucleotide sequence of exons 1-18 below as indicated by alternating underlining, starting with exon 1 underlined, exon 2 not underlined, exon 3 underlined, exon 4 not underlined, and so forth.
SEQ ID NO:75=Exon 1 VTCN1
SEQ ID NO:76=Exon 2 VTCN1
SEQ ID NO:77=Exon 3 VTCN1
SEQ ID NO:78=Exon 4 VTCN1
SEQ ID NO:79=Exon 5 VTCN1
SEQ ID NO:80=Exon 6 VTCN1
SEQ ID NO:81=Exon 1-6 VTCN1
SEQ ID NO:82=Exon 1-2 VTCN1

SEQ ID NO: 81
GTGAGTCACCAAGGAAGGCAGCGGCAGCTCCACTCAGCCAGTACCCAGAT
ACGCTGGGAACCTTCCCCAGCCATGGCTTCCCTGGGGCAGATCCTCTTCT
GGAGCATAATTAGCATCATCATTATTCTGGCTGGAGCAATTGCACTCATC
ATTGGCTTTGGTATTTCAGGGAGACACTCCATCACAGTCACTACTGTCGC
CTCAGCTGGGAACATTGGGGAGGATGGAATCCTGAGCTGCACTTTTGAAC
CTGACATCAAACTTTCTGATATCGTGATACAATGGCTGAAGGAAGGTGTT
TTAGGCTTGGTCCATGAGTTCAAAGAAGGCAAAGATGAGCTGTCGGAGCA
GGATGAAATGTTCAGAGGCCGGACAGCAGTGTTTGCTGATCAAGTGATAG
TTGGCAATGCCTCTTTGCGGCTGAAAAACGTGCAACTCACAGATGCTGGC
ACCTACAAATGTTATATCATCACTTCTAAAGGCAAGGGGAATGCTAACCT
TGAGTATAAAACTGGAGCCTTCAGCATGCCGGAAGTGAATGTGGACTATA
ATGCCAGCTCAGAGACCTTGCGGTGTGAGGCTCCCCGATGGTTCCCCCAG
CCCACAGTGGTCTGGGCATCCCAAGTTGACCAGGGAGCCAACTTCTCGGA
AGTCTCCAATACCAGCTTTGAGCTGAACTCTGAGAATGTGACCATGAAGG
TTGTGTCTGTGCTCTACAATGTTACGATCAACAACACATACTCCTGTATG
ATTGAAAATGACATTGCCAAAGCAACAGGGGATATCAAAGTGACAGAATC
GGAGATCAAAAGGCGGAGTCACCTACAGCTGCTAAACTCAAAGGCTTCTC
TGTGTGTCTCTTCTTTCTTTGCCATCAGCTGGGCACTTCTGCCTCTCAGC
CCTTACCTGATGCTAAAATAATGTGCCTCGGCCACAAAAAAGCATGCAAA
GTCATTGTTACAACAGGGATCTACAGAACTATTTCACCACCAGATATGAC
CTAGTTTTATATTTCTGGGAGGAAATGAATTCATATCTAGAAGTCTGGAG
TGAGCAAACAAGAGCAAGAAACAAAAAGAAGCCAAAAGCAGAAGGCTCCA
ATATGAACAAGATAAATCTATCTTCAAAGACATATTAGAAGTTGGGAAAA
TAATTCATGTGAACTAGACAAGTGTGTTAAGAGTGATAAGTAAAATGCAC
GTGGAGACAAGTGCATCCCCAGATCTCAGGGACCTCCCCCTGCCTGTCAC
CTGGGGAGTGAGAGGACAGGATAGTGCATGTTCTTTGTCTCTGAATTTTT
AGTTATATGTGCTGTAATGTTGCTCTGAGGAAGCCCCTGGAAAGTCTATC
CCAACATATCCACATCTTATATTCCACAAATTAAGCTGTAGTATGTACCC
TAAGACGCTGCTAATTGACTGCCACTTCGCAACTCAGGGGCGGCTGCATT
TTAGTAATGGGTCAAATGATTCACTTTTTATGATGCTTCCAAAGGTGCCT
TGGCTTCTCTTCCCAACTGACAAATGCCAAAGTTGAGAAAAATGATCATA
ATTTTAGCATAAACAGAGCAGTCGGCGACACCGATTTTATAAATAAACTG
AGCACCTTCTTTTTAAACAAACAAATGCGGGTTTATTTCTCAGATGATGT
TCATCCGTGAATGGTCCAGGGAAGGACCTTTCACCTTGTCTATATGGCAT
TATGTCATCACAAGCTCTGAGGCTTCTCCTTTCCATCCTGCGTGGACAGC
TAAGACCTCAGTTTTCAATAGCATCTAGAGCAGTGGGACTCAGCTGGGGT
GATTTCGCCCCCCATCTCCGGGGGAATGTCTGAAGACAATTTTGGTTACC
TCAATGAGGGAGTGGAGGAGGATACAGTGCTACTACCAACTAGTGGATAG
AGGCCAGGGATGCTGCTCAACCTCCTACCATGTACAGGACGTCTCCCCAT
TACAACTACCCAATCCGAAGTGTCAACTGTGTCAGGGCTAAGAAACCCTG
GTTTTGAGTAGAAAAGGGCCTGGAAAGAGGGGAGCCAACAAATCTGTCTG
CTTCCTCACATTAGTCATTGGCAAATAAGCATTCTGTCTCTTTGGCTGCT
GCCTCAGCACAGAGAGCCAGAACTCTATCGGGCACCAGGATAACATCTCT
CAGTGAACAGAGTTGACAAGGCCTATGGGAAATGCCTGATGGGATTATCT
TCAGCTTGTTGAGCTTCTAAGTTTCTTTCCCTTCATTCTACCCTGCAAGC
CAAGTTCTGTAAGAGAAATGCCTGAGTTCTAGCTCAGGTTTTCTTACTCT
GAATTTAGATCTCCAGACCCTGCCTGGCCACAATTCAAATTAAGGCAACA
AACATATACCTTCCATGAAGCACACACAGACTTTTGAAAGCAAGGACAAT
GACTGCTTGAATTGAGGCCTTGAGGAATGAAGCTTTGAAGGAAAAGAATA
CTTTGTTTCCAGCCCCCTTCCCACACTCTTCATGTGTTAACCACTGCCTT
CCTGGACCTTGGAGCCACGGTGACTGTATTACATGTTGTTATAGAAAACT
GATTTTAGAGTTCTGATCGTTCAAGAGAATGATTAAATATACATTTCCTA
CACCA

### CDH1

CDH1-NRG1 fusion sequence
SEQ ID NO: 83
CTGGCTGGAGATTAATCCGGACACTGGTGCCATTTCCACTCGGGCTGAGC
TGGACAGGGAGGATTTTGAGCACGTGAAGAACAGCACGTACACAGCCCTA
ATCATAGCTACAGACAATGCCTTGCCTCCCCGATTGAAAGAGATGAAA

CDH1 sequence information, providing nucleotide sequence of exons 1-18 below as indicated by alternating underlining, starting with exon 1 underlined, exon 2 not underlined, exon 3 underlined, exon 4 not underlined, and so forth.
SEQ ID NO: 84=Exon 1 CDH1
SEQ ID NO: 85=Exon 2 CDH1
SEQ ID NO: 86=Exon 3 CDH1
SEQ ID NO: 87=Exon 4 CDH1
SEQ ID NO: 88=Exon 5 CDH1
SEQ ID NO: 89=Exon 6 CDH1
SEQ ID NO: 90=Exon 7 CDH1
SEQ ID NO: 91=Exon 8 CDH1
SEQ ID NO: 92=Exon 9 CDH1
SEQ ID NO: 93=Exon 10 CDH1
SEQ ID NO: 94=Exon 11 CDH1
SEQ ID NO: 95=Exon 12 CDH1
SEQ ID NO: 96=Exon 13 CDH1
SEQ ID NO: 97=Exon 14 CDH1
SEQ ID NO: 98=Exon 15 CDH1
SEQ ID NO: 99=Exon 16 CDH1
SEQ ID NO: 100=Exon 1-16 CDH1
SEQ ID NO: 101=Exon 1-11 CDH1

SEQ ID NO: 100
AGTGGCGTCGGAACTGCAAAGCACCTGTGAGCTTGCGGAAGTCAGTTCAGACTCCAGCCCGCTCCA
GCCCGGCCCGACCCGACCGCACCCGGCGCCTGCCCTCGCTCGGCGTCCCCGGCCAGCCATGGGCC
CTTGGAGCCGCAGCCTCTCGGCGCTGCTGCTGCTGCTGCAGGTCTCCTCTTGGCTCTGCCAGGAGC
CGGAGCCCTGCCACCCTGGCTTTGACGCCGAGAGCTACACGTTCACGGTGCCCCGGCGCCACCTGG
AGAGAGGCCGCGTCCTGGGCAGAGTGAATTTTGAAGATTGCACCGGTCGACAAAGGACAGCCTATT
TTTCCCTCGACACCCGATTCAAAGTGGGCACAGATGGTGTGATTACAGTCAAAAGGCCTCTACGGTT
TCATAACCCACAGATCCATTTCTTGGTCTACGCCTGGGACTCCACCTACAGAAAGTTTTCCACCAAAG
TCACGCTGAATACAGTGGGGCACCACCACCGCCCCCCGCCCCATCAGGCCTCCGTTTCTGGAATCCA
AGCAGAATTGCTCACATTTCCCAACTCCTCTCCTGGCCTCAGAAGACAGAAGAGAGACTGGGTTATT
CCTCCCATCAGCTGCCCAGAAAATGAAAAAGGCCCATTTCCTAAAAACCTGGTTCAGATCAAATCCAA
CAAAGACAAAGAAGGCAAGGTTTTCTACAGCATCACTGGCCAAGGAGCTGACACACCCCCTGTTGGT
GTCTTTATTATTGAAAGAGAAACAGGATGGCTGAAGGTGACAGAGCCTCTGGATAGAGAACGCATTG
CCACATACACTCTCTTCTCTCACGCTGTGTCATCCAACGGGAATGCAGTTGAGGATCCAATGGAGAT
TTTGATCACGGTAACCGATCAGAATGACAACAAGCCCGAATTCACCCAGGAGGTCTTTAAGGGGTCT
GTCATGGAAGGTGCTCTTCCAGGAACCTCTGTGATGGAGGTCACAGCCACAGACGCGGACGATGAT
GTGAACACCTACAATGCCGCCATCGCTTACACCATCCTCAGCCAAGATCCTGAGCTCCCTGACAAAA
ATATGTTCACCATTAACAGGAACACAGGAGTCATCAGTGTGGTCACCACTGGGCTGGACCGAGAGA
GTTTCCCTACGTATACCCTGGTGGTTCAAGCTGCTGACCTTCAAGGTGAGGGGTTAAGCACAACAGC
AACAGCTGTGATCACAGTCACTGACACCAACGATAATCCTCCGATCTTCAATCCCACCACGTACAAG
GGTCAGGTGCCTGAGAACGAGGCTAACGTCGTAATCACCACACTGAAAGTGACTGATGCTGATGCC
CCCAATACCCCAGCGTGGGAGGCTGTATACACCATATTGAATGATGATGGTGGACAATTTGTCGTCA
CCACAAATCCAGTGAACAACGATGGCATTTTGAAAACAGCAAAGGTTTGTATGGTACCTGGCAAGAT
GCAGAAACTGGCATCCTCACAGCTGTTCATACCCTTGTCCCCTGGGCTTGGATTTTGAGGCCAAGCA
GCAGTACATTCTACACGTAGCAGTGACGAATGTGGTACCTTTTGAGGTCTCTCTCACCACCTCCACA
GCCACCGTCACCGTGGATGTGCTGGATGTGAATGAAGCCCCCATCTTTGTGCCTCCTGAAAAGAGA
GTGGAAGTGTCCGAGGACTTTGGCGTGGGCCAGGAAATCACATCCTACACTGCCCAGGAGCCAGAC
ACATTTATGGAACAGAAAATAACATATCGGATTTGGAGAGACACTGCCAACTGGCTGGAGATTAATC
CGGACACTGGTGCCATTTCCACTCGGGCTGAGCTGGACAGGGAGGATTTTGAGCACGTGAAGAACA
GCACGTACACAGCCCTAATCATAGCTACAGACAATGGTTCTCCAGTTGCTACTGGAACAGGGACACT
TCTGCTGATCCTGTCTGATGTGAATGACAACGCCCCCATACCAGAACCTCGAACTATATTCTTCTGTG
AGAGGAATCCAAAGCCTCAGGTCATAAACATCATTGATGCAGACCTTCCTCCCAATACATCTCCCTTC
ACAGCAGAACTAACACACGGGGCGAGTGCCAACTGGACCATTCAGTACAACGACCCAACCCAAGAA
TCTATCATTTTGAAGCCAAAGATGGCCTTAGAGGTGGGTGACTACAAAATCAATCTCAAGCTCATGG
ATAACCAGAATAAAGACCAAGTGACCACCTTAGAGGTCAGCGTGTGTGACTGTGAAGGGGCCGCTG
GCGTCTGTAGGAAGGCACAGCCTGTCGAAGCAGGATTGCAAATTCCTGCCATTCTGGGGATTCTTG
GAGGAATTCTTGCTTTGCTAATTCTGATTCTGCTGCTCTTGCTGTTTCTTCGGAGGAGAGCGGTGGTC
AAAGAGCCCTTACTGCCCCCAGAGGATGACACCCGGGACAACGTTTATTACTATGATGAAGAAGGA
GGCGGAGAAGAGGACCAGGACTTTGACTTGAGCCAGCTGCACAGGGGCCTGGACGCTCGGCCTGA
AGTGACTCGTAACGACGTTGCACCAACCCTCATGAGTGTCCCCCGGTATCTTCCCCGCCCTGCCAAT
CCCGATGAAATTGGAAATTTTATTGATGAAAATCTGAAAGCGGCTGATACTGACCCCACAGCCCCGC
CTTATGATTCTCTGCTCGTGTTTGACTATGAAGGAAGCGGTTCCGAAGCTGCTAGTCTGAGCTCCCT
GAACTCCTCAGAGTCAGACAAAGACCAGGACTATGACTACTTGAACGAATGGGGCAATCGCTTCAAG
AAGCTGGCTGACATGTACGGAGGCGGCGAGGACGACTAGGGGACTCGAGAGAGGCGGGCCCCAG
ACCCATGTGCTGGGAAATGCAGAAATCACGTTGCTGGTGGTTTTTCAGCTCCCTTCCCTTGAGATGA
GTTTCTGGGGAAAAAAAAGAGACTGGTTAGTGATGCAGTTAGTATAGCTTTATACTCTCTCCACTTTA
TAGCTCTAATAAGTTTGTGTTAGAAAAGTTTCGACTTATTTCTTAAAGCTTTTTTTTTTTTCCCATCACT
CTTTACATGGTGGTGATGTCCAAAAGATACCCAAATTTTAATATTCCAGAAGAACAACTTTAGCATCA
GAAGGTTCACCCAGCACCTTGCAGATTTTCTTAAGGAATTTTGTCTCACTTTTAAAAAGAAGGGGAGA
AGTCAGCTACTCTAGTTCTGTTGTTTTGTGTATATAATTTTTTAAAAAAAATTTGTGTGCTTCTGCTCAT
TACTACACTGGTGTGTCCCTCTGCCTTTTTTTTTTTTTTAAGACAGGGTCTCATTCTATCGGCCAGGCT
GGAGTGCAGTGGTGCAATCACAGCTCACTGCAGCCTTGTCCTCCCAGGCTCAAGCTATCCTTGCACC
TCAGCCTCCCAAGTAGCTGGGACCACAGGCATGCACCACTACGCATGACTAATTTTTTAAATATTTGA
GACGGGGTCTCCCTGTGTTACCCAGGCTGGTCTCAAACTCCTGGGCTCAAGTGATCCTCCCATCTTG
GCCTCCCAGAGTATTGGGATTACAGACATGAGCCACTGCACCTGCCCAGCTCCCCAACTCCCTGCCA
TTTTTTAAGAGACAGTTTCGCTCCATCGCCCAGGCCTGGGATGCAGTGATGTGATCATAGCTCACTG
TAACCTCAAACTCTGGGGCTCAAGCAGTTCTCCCACCAGCCTCCTTTTTATTTTTTTGTACAGATGGG
GTCTTGCTATGTTGCCCAAGCTGGTCTTAAACTCCTGGCCTCAAGCAATCCTTCTGCCTTGGCCCCCC
AAAGTGCTGGGATTGTGGGCATGAGCTGCTGTGCCCAGCCTCCATGTTTTAATATCAACTCTCACTC
CTGAATTCAGTTGCTTTGCCCAAGATAGGAGTTCTCTGATGCAGAAATTATTGGGCTCTTTTAGGGTA
AGAAGTTTGTGTCTTTGTCTGGCCACATCTTGACTAGGTATTGTCTACTCTGAAGACCTTTAATGGCT
TCCCTCTTTCATCTCCTGAGTATGTAACTTGCAATGGGCAGCTATCCAGTGACTTGTTCTGAGTAAGT
GTGTTCATTAATGTTTATTTAGCTCTGAAGCAAGAGTGATATACTCCAGGACTTAGAATAGTGCCTAA
AGTGCTGCAGCCAAAGACAGAGCGGAACTATGAAAAGTGGGCTTGGAGATGGCAGGAGAGCTTGTC
ATTGAGCCTGGCAATTTAGCAAACTGATGCTGAGGATGATTGAGGTGGGTCTACCTCATCTCTGAAA
ATTCTGGAAGGAATGGAGGAGTCTCAACATGTGTTTCTGACACAAGATCCGTGGTTTGTACTCAAAG
CCCAGAATCCCCAAGTGCCTGCTTTTGATGATGTCTACAGAAAATGCTGGCTGAGCTGAACACATTT
GCCCAATTCCAGGTGTGCACAGAAAACCGAGAATATTCAAAATTCCAAATTTTTTTCTTAGGAGCAAG
AAGAAAATGTGGCCCTAAAGGGGGTTAGTTGAGGGGTAGGGGGTAGTGAGGATCTTGATTTGGATC
TCTTTTTATTTAAATGTGAATTTCAACTTTTGACAATCAAAGAAAAGACTTTTGTTGAAATAGCTTTACT
GTTTCTCAAGTGTTTTGGAGAAAAAAATCAACCCTGCAATCACTTTTTGGAATTGTCTTGATTTTTCGG
CAGTTCAAGCTATATCGAATATAGTTCTGTGTAGAGAATGTCACTGTAGTTTTGAGTGTATACATGTG
TGGGTGCTGATAATTGTGTATTTTCTTTGGGGGTGGAAAAGGAAAACAATTCAAGCTGAGAAAAGTA
TTCTCAAAGATGCATTTTTATAAATTTTATTAAACAATTTTGTTAAA

### CXADR

CXADR-NRG1 fusion sequence
SEQ ID NO: 102
ATGGCGCTCCTGCTGTGCTTCGTGCTCCTGTGCGGAGTAGTGGCCTTGCC
TCCCCGATTGAAAGAGATGAAAAGCCAGGAATCGGCTGCAGGTTCCAAAC
T

CXADR sequence information, providing nucleotide sequence of exons 1-5 below as indicated by alternating underlining, starting with exon 1 underlined, exon 2 not underlined, exon 3 underlined, exon 4 not underlined, and so forth.
SEQ ID NO: 103=Exon 1 CXADR
SEQ ID NO: 104=Exon 2 CXADR
SEQ ID NO: 105=Exon 3 CXADR
SEQ ID NO: 106=Exon 4 CXADR
SEQ ID NO: 107=Exon 5 CXADR
SEQ ID NO: 108=Exons 1-5 CXADR

SEQ IN NO: 108
AGTCGGGAGCGCGCGAGGCGCGGGGAGCCTGGGACCAGGAGCGAGAGCCGCCTACCTGCAGCCG
CCGCCCACGGCACGGCAGCCACCATGGCGCTCCTGCTGTGCTTCGTGCTCCTGTGCGGAGTAGTGG
ATTTCGCCAGAAGTTTGAGTATCACTACTCCTGAAGAGATGATTGAAAAAGCCAAAGGGGAAACTGC
CTATCTGCCATGCAAATTTACGCTTAGTCCCGAAGACCAGGGACCGCTGGACATCGAGTGGCTGATA
TCACCAGCTGATAATCAGAAGGTGGATCAAGTGATTATTTTATATTCTGGAGACAAAATTTATGATGA
CTACTATCCAGATCTGAAAGGCCGAGTACATTTTACGAGTAATGATCTCAAATCTGGTGATGCATCAA
TAAATGTAACGAATTTACAACTGTCAGATATTGGCACATATCAGTGCAAAGTGAAAAAAGCTCCTGGT
GTTGCAAATAAGAAGATTCATCTGGTAGTTCTTGTTAAGCCTTCAGGTGCGAGATGTTACGTTGATG
GATCTGAAGAAATTGGAAGTGACTTTAAGATAAAATGTGAACCAAAAGAAGGTTCACTTCCATTACAG
TATGAGTGGCAAAAATTGTCTGACTCACAGAAAATGCCCACTTCATGGTTAGCAGGGAAGATGTGCC
ACCTCCAAAGAGCCGTACGTCCACTGCCAGAAGCTACATCGGCAGTAATCATTCATCCCTGGGGTCC
ATGTCTCCTTCCAACATGGAAGGATATTCCAAGACTCAGTATAACCAAGTACCAAGTGAAGACTTTGA
ACGCACTCCTCAGAGTCCGACTCTCCCACCTGCTAAGGTAGCTGCCCCTAATCTAAGTCGAATGGGT
GCGATTCCTGTGATGATTCCAGCACAGAGCAAGGATGGGTCTATAGTATAGAGCCTCCATATGTCTC
ATCTGTGCTCTCCGTGTTCCTTTCCTTTTTTTGATATATGAAAACCTATTCTGGTCTAAATTGTGTTACT
AGCCTCAAAATACATCAAAAAATAAGTTAATCAGGAACTGTACGGAATATATTTTTAAAAATTTTTGTT
TGGTTATATCGAAATAGTTACAGGCACTAAAGTTAGTAAAGAAAAGTTTACCATCTGAAAAAGCTGGA
TTTTCTTTAAGAGGTTGATTATAAAGTTTTCTAAATTTATCAGTACCTAAGTAAGATGTAGCGCTTTGA
ATATGAAATCATAGGTGAAGACATGGGTGAACTTACTTGCATACCAAGTTGATACTTGAATAACCATC
TGAAAGTGGTACTTGATCATTTTTACCATTATTTTTAGGATGTGTATTTCATTTATTTATGGCCCACCA
GTCTCCCCCAAATTAGTACAGAAATATCCATGACAAAATTACTTACGTATGTTTGTACTTGGTTTTACA
GCTCCTTTGAAAACTCTGTGTTTGGAATATCTCTAAAAACATAGAAAACACTACAGTGGTTTAGAAAT
TACTAATTTTACTTCTAAGTCATTCATAAACCTTGTCTATGAAATGACTTCTTAAATATTTAGTTGATAG
ACTGCTACAGGTAATAGGGACTTAGCAAGCTCTTTTATATGCTAAAGGAGCATCTATCAGATTAAGTT
AGAACATTTGCTGTCAGCCACATATTGAGATGACACTAGGTGCAATAGCAGGGATAGATTTTGTTGG
TGAGTAGTCTCATGCCTTGAGATCTGTGGTGGTCTTCAAAATGGTGGCCAGCCAGATCAAGGATGTA
GTATCTCATAGTTCCCAGGTGATATTTTTCTTATTAGAAAAATATTATAACTCATTTGTTGTTTGACACT
TATAGATTGAAATTTCCTAATTTATTCTAAATTTTAAGTGGTTCTTTGGTTCCAGTGCTTTATGTTGTTG
TTGTTTTTGGATGGTGTTACATATTATATGTTCTAGAAACATGTAATCCTAAATTTACCCTCTTGAATA
TAATCCCTGGATGATATTTTTTATCATAAATGCAGAATAATCAAATACATTTTAAGCAAGTTAAGTGTC
CTCCATCAATTCTGTATTCCAGACTTGGGAGGATGTACAGTTGCTGTTGTGTGATCAAACATGTCTCT
GTGTAGTTCCAGCAAATCAAGCTGAGCTTTGAAAAAGTTTGTCTTAGTTTTGTGAAGGTGATTTATTC
TTAAAAAAAAAAAAGAAAGAAAAAGAAAAAAAGATAAGAAGGAGGAGTAAAGGGACTACTCCTCCTT
GCCAAATGTGCTAAATATCATTTTAGGAGAAGAAAGTGGGTTTATTGTATTTCCCTTAAGATTGTGAG
GGAGTGTGGATACAGTAGAATGAGCCAACAGTTTCTTTATAATAAATACGGTCTGCAATAAATTATTT
CACTAGCTCTAAAACCTTTCCCTAGATTTTAGTAGGGAGTTGGTTTCTGTTAATATCTTTGGGTGCTG
TGGTGGTAAATGCTATATTATGAACGGTGGCATGTATTTACAGTTAGAGTATTGTGTGTACACTTTTT
AATGGTAAACTTAAGCTGAATGTGTAATGGATTTGTGTATAGTTTTACATATTTGGAAGCATTTTAAAA
ACAGGTTTTAACCTTATGTAAAATTACTTTTATACTCGTGTTAACATTTTCATCTGTGCCTTTTGGTAAT
TTAATTTCTATTATGAATTTCTGGTGCCTATGAGCTAGCTATCACCTACCTGAAAGGTGCTTAGAGGT
GAAGGTACTGTTTCTAAAAACACATCACTGTGATACCTTTCTATCCTCACATTTTCAAGCTTGCCTCTT
TTCTGTTCTTTGTGGATATAACTTAAGCAATTGTGTTATTCATAAAGGTTTAGAAATTTCAATATTCCC
AACACTCTATGTTTCTGATTTTATAACAGTAGCCATTTTTGAAAGTCAGATGTTTGGCCTGTTTTATAT
GAATAAAGTTTATTTATAAAATATTATAAAAAATAAGTAAATAAACAGAACATTAATAATAAAGTTTTG
GTTCTTCCTATTCCTGACTTTCATATAATGAAAATTATCCTATTGATCTAAGTAGAAGTTATCATAGAA
AGTGGACACGTATAAGACTTTCCTTCCTTTTTTTTTTTAATAACATATGAGGAACAAGACTTCTCTTCC
CATATACTTCATATTTTAGAGGACATTGTTTTAAAGGCTTATGTCTCACTGTAAAATTCTGTCAGCCAA
ATAGTACCAATACGTTTTCAAGTAGTTCTCACTGATAATTTAGTTGAACCAGAGATCAAATATTTGCTC
CCGAATTACTACTGGTAATCAAGTAGTTGAACAAAAAATTACTAAAGCATTTCCGTTAGATCAGTCAA
GGACAGTACTGCATCTTTTTTTTTTTTTTTTTTTTTAAGACGGAGTCTCGGTCTGTCACCCAGGCTGGA
GTGCAGTGGCGGGATCTCGGCTCACTGCAAGCTCCGCCTCCCAGGTTCACGCCATTCTCCTGCCTCA
GCCTCCCGAGCAGCTGGGACTACAGGCTCCCATCACCACGCTCGGCTAAGTTTTTGTAATTTTAGTA
GAGACAGGGTTTCACCGTGTTAGCCAGGATGGTCTCGATCTCCTGACCTCGTGATCTGCCTGCCTCG
GCCTCCCAAAGTGCTGGGATTACAGGCGTGAACCACTGCACCCGGCCCAGTACTGCATCTTAACAG
CAAAGCCATTTTATTCTACTTTATAACTGAGAGACTTGATACCATCCATCTCTTTAGGTTACAGAGGAT
AATTTGAAGAGAAATGTTACTGTAGAATATATAGTTCTGTACTTTTTTTTTTTTTTTTAAGAGATAGG
GTTTCACTATTGCTCAGGCTGGTCTCAAACTGCTGGGCTCAGGAGATCCTCCTGCCTTGGCCTCCCA
AATTGCTGGGATTACAGGTGTGAGCCGCAGCATCCAGCCAGTTCTGTACTTTGAATATGGAGTAGTT
TACAGCTATTTTTTTTTCTTACTGGTAATCTTAACTAATATGATTCCCTTGTTAGAGAGCCTCTCACTC
CCCCACCCCCAAAAATGTCTACTATTCATGACAGTAACCAATTATTCTGGACAAATTGCTTCTTTTTAA
TTTGAGCTATCTGCCATGGACTTTCTAAAATGGAAACACAGCCTGAGTGTATCTTAGGGAGAGTTTG
ATTGAAAAAATCCAAATCACTATCCATATAGATCATGGATATAAAGAGATACCTGATTTTTATTAAAAA
GATACTTTTTCAAATTTAAGAGTTAATCTTGGAAATTTGGAACAAGTAAAGGGGCAAGTAAACCTTTT
GATGAAATATAAAAGGAACTCATTGCATGAAGTTGACTATCAAATTCTGTGATGTGTGGCTTCTTAAA
AATATTCTCAGTGTCTTTTGTGTGCGTGCAGCATGTACATTTGATGTTATGTGAATGTTGAGTTTTTTC
TTCTAATTTTCACTTCAGCAGTGTTTAGGGCTTTCAGATGCCTTATTCCAGTGTGAACAGAAAAAGTT
CATATTTTATGTGGTTAATGCTTTGATGTGTCACATAAAGAGTAGTTTGTAGAAAATGTTGGCACAAT
TTTAACTTCTTAGTGGCTTGTGACATTATATATTATATATATATATGTACATATATCTTTATAACATTCC
TGTGTTTAGTAGTGTAAATGTTCTGGGCAAGTTTTAATATTTTGAATGCCTTTGGATATTCCAGCAATA
AAGGCATCATGTTCTGCAATAGGATTTCTTACTCATTTACCTATTTTAACACTAAAATAGACCACAACT
GAGCACAAATTCCTTTTATAAATGTTATAGAAGCAGGGAAGAATAATAAACACATTTGTGAATTGTGG
TTCAGTTTATTTATCTTTAGGGAAGGCTGATCATTTATCTTATAGCAGATAACCCCAGCCTCTTATTCA
TTATGGTTAACTTTTATAATTTATCTTATTTTATAATTTAAGAATATAGTACATATCAGTTGGGTTTGGT
TTTGGTCATCGAGACTAAAAGCTCCATCAAAACAGAACTTTGTGTTTTCTGCTAACTTATTTAATGACA
CAAGTTTTAAGAGAACCACAATTCATTGATTCACTTATTCTTTTCCCTAATTGTGAATTTTAGTGATAA
ATACACCTGTACTACTGAGGAAAATATTCTGACACTTCACGTGTGCAAAGTATAGAACTGACAGTGTC
AGTTTCAGATTTTGTATGTACGATTTCTGGCTTATATATCCAATGGTGCAGATTTTGAAATTTGTAAGA
ACAAAATTTGTTAAGAAAAACAACTTGCTCTAGTTTTGTGACCTTGTGTACTTTTGAAATAAAATCAAG
AAAGCAGT

### GTF2E2

GTF2E2-NRG1 fusion sequence
SEQ ID NO: 109
GGGAGCTGTTCAAAAAACGAGCTCTTTCTACTCCTGTAGTAGAAAAACGT
TCAGCATCTTCTGAGTCATCATCATCATCGTCAAAGAAGAAGAAAACAAA
GGTAGAACATGGAGGATCGTCAGGCTCTAAACAAAATTCTGCCTTGCCTC
CCCGATTGAAAGAGATGAAAAGCCAGGAATCGGCTGCAGGTTCCAAACTA
GTCCTTCGGTGTGAAACCAGTTCTGAATACTCCTCTCTCAGATTCAAGTG
GTTCAAGAATGGGAATGA

GTF2E2 sequence information, providing nucleotide sequence of exons 1-8 below as indicated by alternating underlining, starting with exon 1 underlined, exon 2 not underlined, exon 3 underlined, exon 4 not underlined, and so forth.
SEQ ID NO: 110=Exon 1 GTF2E2
SEQ ID NO: 111=Exon 2 GTF2E2
SEQ ID NO: 112=Exon 3 GTF2E2
SEQ ID NO: 113=Exon 4 GTF2E2
SEQ ID NO: 114=Exon 5 GTF2E2
SEQ ID NO: 115=Exon 6 GTF2E2
SEQ ID NO: 116=Exon 7 GTF2E2
SEQ ID NO: 117=Exon 8 GTF2E2
SEQ ID NO: 118=Exon 1-8 GTF2E2
SEQ ID NO: 119=Exon 1-2 GTF2E2

SEQ IN NO: 118
ACTGAGCTCCTAGCACCCGATCGGGAAGTGGCGGGCGGAGTCCCGGGTCC
AGTCGCCGCCTCAGCTACCGCCGCTGCCGCCGCCGCCGCCGCCACCGCCA
GTGGTGAGACCCCGACCTGGCGGGTCAGCGCTGGGCGTGCGTGCGGGCAG
GCGGGGGCGCTGACGAGAAGCAGGAAGAGGGTGCAGTGCCGGCGTGGGCG
GCCGGCCGAGGCGGAGGCGCAGGAAGGGGGCGGCGAGTCGTGCGAGGCTG
CCCTTCTCACTCAGCATTATGGATCCAAGCCTGTTGAGAGAAAGGGAGCT
GTTCAAAAAACGAGCTCTTTCTACTCCTGTAGTAGAAAAACGTTCAGCAT
CTTCTGAGTCATCATCATCATCGTCAAAGAAGAAGAAAACAAAGGTAGAA
CATGGAGGATCGTCAGGCTCTAAACAAAATTCTGATCATAGCAATGGATC
ATTTAACTTGAAAGCTTTGTCAGGAAGCTCTGGATATAAGTTTGGTGTTC
TTGCTAAGATTGTGAATTACATGAAGACACGGCATCAGCGAGGAGATACG
CATCCTCTAACCTTAGATGAAATTTTGGATGAAACACAACATTTAGATAT
TGGACTCAAGCAGAAACAATGGCTAATGACTGAGGCTTTAGTCAACAATC
CCAAAATTGAAGTAATAGATGGGAAGTATGCTTTCAAGCCCAAGTACAAC
GTGAGAGATAAGAAGGCCCTACTTAGGCTCTTAGATCAGCATGACCAGCG
AGGATTAGGAGGAATTCTTTTAGAAGACATAGAAGAAGCACTGCCCAATT
CCCAGAAAGCTGTCAAGGCTTTGGGGGACCAGATACTATTTGTAAATCGT
CCCGATAAGAAGAAAATACTTTTCTTCAATGATAAGAGCTGTCAGTTTTC
TGTGGATGAAGAATTTCAGAAACTGTGGAGGAGTGTCACTGTAGATTCCA
TGGACGAGGAGAAAATTGAAGAATATCTGAAGCGACAGGGTATTTCTTCC
ATGCAGGAATCTGGACCAAAGAAAGTGGCCCCTATTCAGAGAAGGAAAAA
GCCTGCTTCACAGAAAAAGCGACGCTTTAAGACTCATAACGAACACTTGG
CTGGAGTGCTGAAGGATTACTCTGACATTACTTCCAGCAAATAGGGAACA
GTTTTGCCCTGGAACAGAGTTACAGATACACAATCAAGAGTGTTCTTGCT
GATGCTCGGGGTCTGAAGACTGTCTTCCTATCTGCTTCTTGCGGCTGAGG
AGAGGAGCAGTTCAGTTTACAAAACAAGTGCAAATTACCAAACTCAAAGC
TTATTTGAGTAGAATGGGCTCATGGGCAATGTGATGTTCCCTGTTAACCT
TCTGTTACTCCCTGGGAGAAAGGCGCTGAGCGTGGCATGCAGGTGTCTTT
GCTGTGTTTTTCTCCACTTCTAAATGGTTCCTGGTTCCTTTCTTCCTCGT
TTGTTACTTTAGAGCAAGTTTGCCCATAGTCTTGAATGCAATATTTGTTT
ATTCCAAAAGAACATATTTATAATAAAATCACTGTAGAAGGATTTTTAAG
ATGTTAGTGAATTCTGTTTCTTTTCATTCTCGGAAATGGCAGGAAGCAGC
TCCAGTCTCTGATTTCCATGGGTCACGTGCTGGGGATGTGATGAAGCCTG
CAGTCTGCACTGTGTTGCTGAGCACATGGATTTCACCACTGGAACACAGG
TGTGCTGCTTGTTAGCAAGCAGAGCAATAAAGATGTGCTGGATGTCA

### CSMD1

CSMD1-NRG1 fusion sequence
SEQ ID NO: 120
ATCCTAAACAGCACATCCAATCACCTGTGGCTAGAGTTCAACACCAATGG
ATCTGACACCGACCAAGGTTTTCAACTCACCTATACCACTACATCTACAT
CCACCACTGGGACAAGCCATCTTGTAAAATGTGCGGAGAAGGAGAAAACT

CSMD1 sequence information, providing nucleotide sequence of exons 1-23 below as indicated by alternating underlining, starting with exon 1 underlined, exon 2 not underlined, exon 3 underlined, exon 4 not underlined, and so forth up until and including exon 23.
SEQ ID NO: 121=Exon 1 CSMD1
SEQ ID NO: 122=Exon 2 CSMD1
SEQ ID NO: 123=Exon 3 CSMD1
SEQ ID NO: 124=Exon 4 CSMD1
SEQ ID NO: 125=Exon 5 CSMD1
SEQ ID NO: 126=Exon 6 CSMD1
SEQ ID NO: 127=Exon 7 CSMD1
SEQ ID NO: 128=Exon 8 CSMD1
SEQ ID NO: 129=Exon 9 CSMD1
SEQ ID NO: 130=Exon 10 CSMD1
SEQ ID NO: 131=Exon 11 CSMD1
SEQ ID NO: 132=Exon 12 CSMD1
SEQ ID NO: 133=Exon 13 CSMD1
SEQ ID NO: 134=Exon 14 CSMD1
SEQ ID NO: 135=Exon 15 CSMD1
SEQ ID NO: 136=Exon 16 CSMD1
SEQ ID NO: 137=Exon 17 CSMD1
SEQ ID NO: 138=Exon 18 CSMD1
SEQ ID NO: 139=Exon 19 CSMD1
SEQ ID NO: 140=Exon 20 CSMD1
SEQ ID NO: 141=Exon 21 CSMD1
SEQ ID NO: 142=Exon 22 CSMD1
SEQ ID NO: 143=Exon 23 CSMD1
SEQ IN NO: 144=Exon 1-70 CSMD1
SEQ ID NO: 145=Exon 1-23 CSMD1

SEQ ID NO: 144
CTCCACGGCAGCGGCTCCTTGTGCCACTAGCAGCCCTTCTTCTGCGCTCTCCGCCTTTTCTCTCTAGA
CTGGATCTCTCCTCCCCCCCGCGCCCCCCTCCCCGCATCTCCCACTCGCTGGCTCTCTCTCCAGCTG
CCTCCTCTCCAGGTCTCTCCTGGCTGCGCGCGCTCCTCTCCCCGCTTCTCCCCCTCCCGCAGCCTCG
CCGCCTTGGTGCCTTCCTGCCCGGCTCGGCCGGCGCTCGTCCCCGGCCCCGGCCCCGCCAGCCCG
GGTCTCCGCGCTCGGAGCAGCTCAGCCCTGCAGTGGCTCGGGACCCGATGCTATGAGAGGGAAGC
GAGCCGGGCGCCCAGACCTTCAGGAGGCGTCGGATGCGCGGCGGGTCTTGGGACCGGGCTCTCTC
TCCGGCTCGCCTTGCCCTCGGGTGATTATTTGGCTCCGCTCATAGCCCTGCCTTCCTCGGAGGAGCC
ATCGGTGTCGCGTGCGTGTGGAGTATCTGCAGACATGACTGCGTGGAGGAGATTCCAGTCGCTGCT
CCTGCTTCTCGGGCTGCTGGTGCTGTGCGCGAGGCTCCTCACTGCAGCGAAGGGTCAGAACTGTGG
AGGCTTAGTCCAGGGTCCCAATGGCACTATTGAGAGCCCAGGGTTTCCTCACGGGTATCCGAACTAT
GCCAACTGCACCTGGATCATCATCACGGGCGAGCGCAATAGGATACAGTTGTCCTTCCATACCTTTG
CTCTTGAAGAAGATTTTGATATTTTATCAGTTTACGATGGACAGCCTCAACAAGGGAATTTAAAAGTG
AGATTATCGGGATTTCAGCTGCCCTCCTCTATAGTGAGTACAGGATCTATCCTCACTCTGTGGTTCAC
GACAGACTTCGCTGTGAGTGCCCAAGGTTTCAAAGCATTATATGAAGTTTTACCTAGCCACACTTGTG
GAAATCCTGGAGAAATCCTGAAAGGAGTTCTGCATGGAACGAGATTCAACATAGGAGACAAAATCCG
GTACAGCTGCCTCCCTGGCTACATCTTGGAAGGCCACGCCATCCTGACCTGCATCGTCAGCCCAGGA
AATGGTGCATCGTGGGACTTCCCAGCTCCCTTTTGCAGAGCTGAGGGAGCCTGCGGAGGAACCTTA
CGCGGGACCAGCAGCTCCATCTCCAGCCCGCACTTCCCTTCAGAGTACGAGAACAACGCGGACTGC
ACCTGGACCATTCTGGCTGAGCCCGGGGACACCATTGCGCTGGTCTTCACTGACTTTCAGCTAGAAG
AAGGATATGATTTCTTAGAGATCAGTGGCACGGAAGCTCCATCCATATGGCTAACTGGCATGAACCT
CCCCTCTCCAGTTATCAGTAGCAAGAATTGGCTACGACTCCATTTCACCTCTGACAGCAACCACCGAC
GCAAAGGATTTAACGCTCAGTTCCAAGTGAAAAAGGCGATTGAGTTGAAGTCAAGAGGAGTCAAGAT
GCTGCCCAGCAAGGATGGAAGCCATAAAAACTCTGTCTTGAGCCAAGGAGGTGTTGCATTGGTCTCT
GACATGTGTCCAGATCCTGGGATTCCAGAAAATGGTAGAAGAGCAGGTTCCGACTTCAGGGTTGGT
GCAAATGTACAGTTTTCATGTGAGGACAATTACGTGCTCCAGGGATCTAAAAGCATCACCTGTCAGA
GAGTTACAGAGACGCTCGCTGCTTGGAGTGACCACAGGCCCATCTGCCGAGCGAGAACATGTGGAT
CCAATCTGCGTGGGCCCAGCGGCGTCATTACCTCCCCTAATTATCCGGTTCAGTATGAAGATAATGC
ACACTGTGTGTGGGTCATCACCACCACCGACCCGGACAAGGTCATCAAGCTTGCCTTTGAAGAGTTT
GAGCTGGAGCGAGGCTATGACACCCTGACGGTTGGTGATGCTGGGAAGGTGGGAGACACCAGATC
GGTCTTGTACGTGCTCACGGGATCCAGTGTTCCTGACCTCATTGTGAGCATGAGCAACCAGATGTGG
CTACATCTGCAGTCGGATGATAGCATTGGCTCACCTGGGTTTAAAGCTGTTTACCAAGAAATTGAAA
AGGGAGGGTGTGGGGATCCTGGAATCCCCGCCTATGGGAAGCGGACGGGCAGCAGTTTCCTCCAT
GGAGATACACTCACCTTTGAATGCCCGGCGGCCTTTGAGCTGGTGGGGGAGAGAGTTATCACCTGT
CAGCAGAACAATCAGTGGTCTGGCAACAAGCCCAGCTGTGTATTTTCATGTTTCTTCAACTTTACGGC
ATCATCTGGGATTATTCTGTCACCAAATTATCCAGAGGAATATGGGAACAACATGAACTGTGTCTGGT
TGATTATCTCGGAGCCAGGAAGTCGAATTCACCTAATCTTTAATGATTTTGATGTTGAGCCTCAGTTT
GACTTTCTCGCGGTCAAGGATGATGGCATTTCTGACATAACTGTCCTGGGTACTTTTTCTGGCAATGA
AGTGCCTTCCCAGCTGGCCAGCAGTGGGCATATAGTTCGCTTGGAATTTCAGTCTGACCATTCCACT
ACTGGCAGAGGGTTCAACATCACTTACACCACATTTGGTCAGAATGAGTGCCATGATCCTGGCATTC
CTATAAACGGACGACGTTTTGGTGACAGGTTTCTACTCGGGAGCTCGGTTTCTTTCCACTGTGATGA
TGGCTTTGTCAAGACCCAGGGATCCGAGTCCATTACCTGCATACTGCAAGACGGGAACGTGGTCTG
GAGCTCCACCGTGCCCCGCTGTGAAGCTCCATGTGGTGGACATCTGACAGCGTCCAGCGGAGTCAT
TTTGCCTCCTGGATGGCCAGGATATTATAAGGATTCTTTACATTGTGAATGGATAATTGAAGCAAAAC
CAGGCCACTCTATCAAAATAACTTTTGACAGATTTCAGACAGAGGTCAATTATGACACCTTGGAGGTC
AGAGATGGGCCAGCCAGTTCGTCCCCACTGATCGGCGAGTACCACGGCACCCAGGCACCCCAGTTC
CTCATCAGCACCGGGAACTTCATGTACCTGCTGTTCACCACTGACAACAGCCGCTCCAGCATCGGCT
TCCTCATCCACTATGAGAGTGTGACGCTTGAGTCGGATTCCTGCCTGGACCCGGGCATCCCTGTGAA
CGGCCATCGCCACGGTGGAGACTTTGGCATCAGGTCCACAGTGACTTTCAGCTGTGACCCGGGGTA
CACACTAAGTGACGACGAGCCCCTCGTCTGTGAGAGGAACCACCAGTGGAACCACGCCTTGCCCAG
CTGCGACGCTCTATGTGGAGGCTACATCCAAGGGAAGAGTGGAACAGTCCTTTCTCCTGGGTTTCCA
GATTTTTATCCAAACTCTCTAAACTGCACGTGGACCATTGAAGTGTCTCATGGGAAAGGAGTTCAAAT
GATCTTTCACACCTTTCATCTTGAGAGTTCCCACGACTATTTACTGATCACAGAGGATGGAAGTTTTT
CCGAGCCCGTTGCCAGGCTCACCGGGTCGGTGTTGCCTCATACGATCAAGGCAGGCCTGTTTGGAA
ACTTCACTGCCCAGCTTCGGTTTATATCAGACTTCTCAATTTCGTACGAGGGCTTCAATATCACATTTT
CAGAATATGACCTGGAGCCATGTGATGATCCTGGAGTCCCTGCCTTCAGCCGAAGAATTGGTTTTCA
CTTTGGTGTGGGAGACTCTCTGACGTTTTCCTGCTTCCTGGGATATCGTTTAGAAGGTGCCACCAAG
CTTACCTGCCTGGGTGGGGGCCGCCGTGTGTGGAGTGCACCTCTGCCAAGGTGTGTGGCCGAATGT
GGAGCAAGTGTCAAAGGAAATGAAGGAACATTACTGTCTCCAAATTTTCCATCCAATTATGATAATAA
CCATGAGTGTATCTATAAAATAGAAACAGAAGCCGGCAAGGGCATCCACCTTAGAACACGAAGCTTC
CAGCTGTTTGAAGGAGATACTCTAAAGGTATATGATGGAAAAGACAGTTCCTCACGTCCACTGGGCA
CGTTCACTAAAAATGAACTTCTGGGGCTGATCCTAAACAGCACATCCAATCACCTGTGGCTAGAGTT
CAACACCAATGGATCTGACACCGACCAAGGTTTTCAACTCACCTATACCAGTTTTGATCTGGTAAAAT
GTGAGGATCCGGGCATCCCTAACTACGGCTATAGGATCCGTGATGAAGGCCACTTTACCGACACTGT
AGTTCTGTACAGTTGCAACCCGGGGTACGCCATGCATGGCAGCAACACCCTGACCTGTTTGAGTGG
AGACAGGAGAGTGTGGGACAAACCACTACCTTCGTGCATAGCGGAATGTGGTGGTCAGATCCATGC
AGCCACATCAGGACGAATATTGTCCCCTGGCTATCCAGCTCCGTATGACAACAACCTCCACTGCACC
TGGATTATAGAGGCAGACCCAGGAAAGACCATTAGCCTCCATTTCATTGTTTTCGACACGGAGATGG
CTCACGACATCCTCAAGGTCTGGGACGGGCCGGTGGACAGTGACATCCTGCTGAAGGAGTGGAGTG
GCTCCGCCCTTCCGGAGGACATCCACAGCACCTTCAACTCACTCACCCTGCAGTTCGACAGCGACTT
CTTCATCAGCAAGTCTGGCTTCTCCATCCAGTTCTCCACCTCAATTGCAGCCACCTGTAACGATCCAG
GTATGCCCCAAAATGGCACCCGCTATGGAGACAGCAGAGAGGCTGGAGACACCGTCACATTCCAGT
GTGACCCTGGCTATCAGCTCCAAGGACAAGCCAAAATCACCTGTGTGCAGCTGAATAACCGGTTCTT
TTGGCAACCAGACCCTCCTACATGCATAGCTGCTTGTGGAGGGAATCTGACGGGCCCAGCAGGTGT
TATTTTGTCACCCAACTACCCACAGCCGTATCCTCCTGGGAAGGAATGTGACTGGAGAGTAAAAGTG
AACCCGGACTTTGTCATCGCCTTGATATTCAAAAGTTTCAACATGGAGCCCAGCTATGACTTCCTACA
CATCTATGAAGGGGAAGATTCCAACAGCCCCCTCATTGGGAGTTACCAGGGCTCTCAGGCCCCAGA
AAGAATAGAGAGTAGCGGAAACAGCCTGTTTCTGGCATTTCGGAGTGATGCCTCCGTGGGCCTTTCA
GGGTTCGCCATTGAATTTAAAGAGAAACCACGGGAAGCTTGTTTTGACCCAGGAAATATAATGAATG
GGACAAGAGTTGGAACAGACTTCAAGCTTGGCTCCACCATCACCTACCAGTGTGACTCTGGCTATAA
GATTCTTGACCCCTCATCCATCACCTGTGTGATTGGGGCTGATGGGAAACCCTCCTGGGACCAAGTG
CTGCCCTCCTGCAATGCTCCCTGTGGAGGCCAGTACACGGGATCAGAAGGGGTAGTTTTATCACCAA
ACTACCCCCATAATTACACAGCTGGTCAAATATGCCTCTATTCCATCACGGTACCAAAGGAATTCGTG
GTCTTTGGACAGTTTGCCTATTTCCAGACAGCCCTGAATGATTTGGCAGAATTATTTGATGGAACCCA
TGCACAGGCCAGACTTCTCAGCTCACTCTCGGGGTCTCACTCAGGGGAAACATTGCCCTTGGCTACG
TCAAATCAAATTCTGCTCCGATTCAGTGCAAAGAGCGGTGCCTCTGCCCGCGGCTTCCACTTCGTGT
ATCAAGCTGTTCCTCGTACCAGTGACACCCAATGCAGCTCTGTCCCCGAGCCCAGATACGGAAGGA
GAATTGGTTCTGAGTTTTCTGCCGGCTCCATCGTCCGATTCGAGTGCAACCCGGGATACCTGCTTCA
GGGTTCCACGGCGCTCCACTGCCAGTCCGTGCCCAACGCCTTGGCACAGTGGAACGACACGATCCC
CAGCTGTGTGGTACCCTGCAGTGGCAATTTCACTCAACGAAGAGGTACAATCCTGTCCCCCGGCTAC
CCTGAGCCATACGGAAACAACTTGAACTGTATATGGAAGATCATAGTTACGGAGGGCTCGGGAATTC
AGATCCAAGTGATCAGTTTTGCCACGGAGCAGAACTGGGACTCCCTTGAGATCCACGATGGTGGGG
ATGTGACCGCACCCAGACTGGGAAGCTTCTCAGGCACCACAGTACCGGCACTGCTGAACAGTACTT
CCAACCAACTCTACCTGCATTTCCAGTCTGACATTAGTGTGGCAGCTGCTGGTTTCCACCTGGAATAC
AAAACTGTAGGTCTTGCTGCATGCCAAGAACCAGCCCTCCCCAGCAACAGCATCAAAATCGGAGATC
GGTACATGGTGAACGACGTGCTCTCCTTCCAGTGCGAGCCCGGGTACACCCTGCAGGGCCGTTCCC
ACATTTCCTGTATGCCAGGGACCGTTCGCCGTTGGAACTATCCGTCTCCCCTGTGCATTGCAACCTG
TGGAGGGACGCTGAGCACCTTGGGTGGTGTGATCCTGAGCCCCGGCTTCCCAGGTTCTTACCCCAA
CAACTTAGACTGCACCTGGAGGATCTCATTACCCATCGGCTATGGTGCACATATTCAGTTTCTGAATT
TTTCTACCGAAGCTAATCATGACTTCCTTGAAATTCAAAATGGACCTTACCACACCAGCCCCATGATT
GGACAATTTAGCGGCACGGATCTCCCCGCGGCCCTGCTGAGCACAACGCATGAAACCCTCATCCAC
TTTTATAGTGACCATTCGCAAAACCGGCAAGGATTTAAACTTGCTTACCAAGCCTATGAATTACAGAA
CTGTCCAGATCCACCCCCATTTCAGAATGGGTACATGATCAACTCGGATTACAGCGTGGGGCAATCA
GTATCTTTCGAGTGTTATCCTGGGTACATTCTAATAGGCCATCCTGTCCTCACTTGTCAGCATGGGAT
CAACAGAAACTGGAACTACCCTTTTCCAAGATGTGATGCCCCTTGTGGGTACAACGTAACTTCTCAG
AACGGCACCATCTACTCCCCTGGCTTTCCTGATGAGTATCCGATCCTGAAGGACTGCATTTGGCTCA
TCACGGTGCCTCCAGGGCACGGAGTTTACATCAACTTCACCCTGTTACAGACGGAAGCTGTCAACGA
TTACATTGCTGTTTGGGACGGTCCCGATCAGAACTCACCCCAGCTGGGAGTTTTCAGTGGCAACACA
GCCCTCGAAACGGCGTATAGCTCCACCAACCAAGTCCTGCTCAAGTTCCACAGCGACTTTTCAAATG
GAGGCTTCTTTGTCCTCAATTTCCACGCATTTCAGCTCAAGAAATGTCAACCTCCCCCAGCGGTTCCA
CAGGCAGAAATGCTTACTGAGGATGATGATTTCGAAATAGGAGATTTTGTGAAGTACCAGTGCCACC
CCGGGTACACCTTGGTGGGGACCGACATTCTGACTTGCAAGCTCAGTTCCCAGTTGCAGTTTGAGG
GTTCTCTCCCAACATGTGAAGCACAATGCCCAGCAAATGAAGTCCGGACTGGATCATCGGGAGTCAT
TCTCAGTCCAGGGTATCCGGGTAATTATTTTAACTCCCAGACTTGCTCTTGGAGTATTAAAGTGGAAC
CAAACTACAACATTACCATCTTTGTGGACACATTTCAAAGTGAAAAGCAGTTTGATGCACTGGAAGTG
TTTGATGGTTCTTCTGGGCAAAGTCCTCTGCTAGTAGTCTTAAGTGGGAATCATACTGAACAATCAAA
TTTTACAAGCAGGAGTAATCAGTTATATCTCCGCTGGTCCACTGACCATGCCACCAGTAAGAAAGGA
TTCAAGATTCGCTATGCAGCACCTTACTGCAGTTTGACCCACCCCCTGAAGAATGGGGGTATTCTAA
ACAGGACTGCAGGAGCGGTTGGAAGCAAAGTGCATTATTTTTGCAAGCCTGGATACCGAATGGTCG
GCCACAGCAATGCAACCTGTAGACGAAACCCACTTGGCATGTACCAGTGGGACTCCCTCACGCCACT
CTGCCAGGCTGTGTCCTGTGGAATCCCAGAATCCCCAGGAAACGGTTCATTTACCGGGAACGAGTTC
ACTTTGGACAGTAAAGTGGTCTATGAATGTCATGAGGGCTTCAAGCTTGAATCCAGCCAGCAAGCAA
CAGCCGTGTGTCAAGAAGATGGGTTGTGGAGTAACAAGGGGAAGCCGCCCACGTGTAAGCCGGTC
GCTTGCCCCAGCATTGAAGCTCAGCTCTCAGAACATGTCATCTGGAGGCTGGTTTCAGGATCCTTGA
ATGAGTACGGTGCTCAAGTATTGCTGAGCTGCAGTCCTGGTTACTACTTAGAAGGCTGGAGGCTCCT
GCGGTGCCAGGCCAATGGGACGTGGAACATAGGAGATGAGAGGCCAAGCTGTCGAGTTATCTCGT
GTGGAAGCCTTTCCTTTCCCCCAAATGGCAACAAGATTGGAACGTTGACAGTTTATGGGGCCACAGC
TATATTTACGTGCAACACCGGCTACACGCTTGTGGGGTCTCATGTCAGAGAGTGCTTGGCAAATGGG
CTCTGGAGCGGCAGCGAAACTCGATGTCTGGCTGGCCACTGCGGTTCCCCAGACCCGATTGTGAAC
GGTCACATTAGTGGAGATGGCTTCAGTTACAGAGACACGGTGGTTTACCAGTGCAATCCTGGTTTCC
GGCTTGTGGGAACTTCCGTGAGGATATGCCTGCAAGACCACAAGTGGTCTGGACAAACGCCTGTCT
GTGTCCCCATCACATGTGGTCACCCTGGAAACCCTGCCCACGGATTCACTAATGGCAGTGAGTTCAA
CCTGAATGATGTCGTGAATTTCACCTGCAACACGGGCTATTTGCTGCAGGGCGTGTCTCGAGCCCAG
TGTCGGAGCAACGGCCAGTGGAGTAGCCCTCTGCCCACGTGTCGAGTGGTGAACTGTTCTGATCCA
GGCTTTGTGGAAAATGCCATTCGTCACGGGCAACAGAACTTCCCTGAGAGTTTTGAGTATGGAATGA
GTATCCTGTACCATTGCAAGAAGGGATTTTACTTGCTGGGATCTTCAGCCTTGACCTGTATGGCAAAT
GGCTTATGGGACCGATCCCTGCCCAAGTGTTTGGCTATATCGTGTGGACACCCAGGGGTCCCTGCC
AACGCCGTCCTCACTGGAGAGCTGTTTACCTATGGCGCCGTCGTGCACTACTCCTGCAGAGGGAGC
GAGAGCCTCATAGGCAACGACACGAGAGTGTGCCAGGAAGACAGTCACTGGAGCGGGGCACTGCC
CCACTGCACAGGAAATAATCCTGGATTCTGTGGTGATCCGGGGACCCCAGCACATGGGTCTCGGCT
TGGTGATGACTTTAAGACAAAGAGTCTTCTCCGCTTCTCCTGTGAAATGGGGCACCAGCTGAGGGGC
TCCCCTGAACGCACGTGTTTGCTCAATGGGTCATGGTCAGGACTGCAGCCGGTGTGTGAGGCCGTG
TCCTGTGGCAACCCTGGCACACCCACCAACGGAATGATTGTCAGTAGTGATGGCATTCTGTTCTCCA
GCTCGGTCATCTATGCCTGCTGGGAAGGCTACAAGACCTCAGGGCTCATGACACGGCATTGCACAG
CCAATGGGACCTGGACAGGCACTGCTCCCGACTGCACAATTATAAGTTGTGGGGATCCAGGCACAC
TAGCAAATGGCATCCAGTTTGGGACCGACTTCACCTTCAACAAGACTGTGAGCTATCAGTGTAACCC
AGGCTATGTCATGGAAGCAGTCACATCCGCCACTATTCGCTGTACCAAAGACGGCAGGTGGAATCC
GAGCAAACCTGTCTGCAAAGCCGTGCTGTGTCCTCAGCCGCCGCCGGTGCAGAATGGAACAGTGGA
GGGAAGTGATTTCCGCTGGGGCTCCAGCATAAGTTACAGCTGCATGGACGGTTACCAGCTCTCTCAC
TCCGCCATCCTCTCCTGTGAAGGTCGCGGGGTGTGGAAAGGAGAGATCCCCCAGTGTCTCCCTGTG
TTCTGCGGAGACCCTGGCATCCCCGCAGAAGGGCGACTTAGTGGGAAAAGTTTCACCTATAAGTCC
GAAGTCTTCTTCCAGTGCAAATCTCCATTTATACTCGTGGGATCCTCCAGAAGAGTCTGCCAAGCTGA
CGGCACGTGGAGCGGCATACAACCCACCTGCATTGATCCTGCTCATAACACCTGCCCAGACCCTGGT
ACGCCACACTTTGGAATACAGAATAGCTCCAGAGGCTATGAGGTTGGAAGCACGGTTTTTTTCAGGT
GCAGAAAAGGCTACCATATTCAAGGTTCCACGACTCGCACCTGCCTTGCCAATTTAACATGGAGTGG
GATACAGACCGAATGTATACCTCATGCCTGCAGACAGCCAGAAACCCCGGCACACGCGGATGTGAG
AGCCATCGATCTTCCTACTTTCGGCTACACCTTAGTGTACACCTGCCATCCAGGCTTTTTCCTCGCAG
GGGGATCTGAGCACAGAACATGTAAAGCAGACATGAAATGGACAGGAAAGTCGCCTGTGTGTAAAA
GTAAAGGAGTGAGAGAAGTTAATGAAACAGTTACTAAAACTCCAGTTCCTTCAGATGTCTTTTTCGTC
AATTCACTGTGGAAGGGGTATTATGAATATTTAGGGAAAAGACAACCCGCCACTCTAACTGTTGACT
GGTTCAATGCAACAAGCAGTAAGGTGAATGCCACCTTCAGCGAAGCCTCGCCAGTGGAGCTGAAGT
TGACAGGCATTTACAAGAAGGAGGAGGCCCACTTACTCCTGAAAGCTTTTCAAATTAAAGGCCAGGC
AGATATTTTTGTAAGCAAGTTCGAAAATGACAACTGGGGACTAGATGGTTATGTGTCATCTGGACTT
GAAAGAGGAGGATTTACTTTTCAAGGTGACATTCATGGAAAAGACTTTGGAAAATTTAAGCTAGAAA
GGCAAGATCCTTTAAACCCAGATCAAGACTCTTCCAGTCATTACCACGGCACCAGCAGTGGCTCTGT
GGCGGCTGCCATTCTGGTTCCTTTCTTTGCTCTAATTTTATCAGGGTTTGCATTTTACCTCTACAAACA
CAGAACGAGACCAAAAGTTCAATACAATGGCTATGCTGGGCATGAAAACAGCAATGGACAAGCATC
GTTTGAAAACCCCATGTATGATACAAACTTAAAACCCACAGAAGCCAAGGCTGTGAGGTTTGACACA
ACTCTGAACACAGTCTGTACAGTGGTATAGCCCTCAGTGCCCCAACAGGACTGATTCATAGCCATAC
CTCTGATGGACAAGCAGTGATTCCTTTGGTGCCATATACCACTCTCCCTTCCACTCTGGCTTTACTGC
AGCGATCTTCAACCTTGTCTACTGGCATAAGTGCAGCGGGGATCTCTACTCAAATGTGTCAGGGTCT
TCTACGGATCAAACTACACATGCGTTTTCATTCCAAAAGTGGGTTCTAAATGCCTGGCTGCATCTGTA
TGAAATCAAGGCACACTCCAGGAAGACTGCCACGTCGCGCCAACACGTCATACTCAATGCCTCAGAC
TTTCATATTTCTGTGTTGCTGAGATGCCTTTCAATGCAATCGTCTGGGCTCGTGGATATGTCCCTCAG
GTGCGGTGACAGAATGGTGGCACCACGATATGTGTTCTCTTGTGTTGTTTTTCCTTTTTAAACCCCCA
TGAACACGAATACTCTGAAAAAAATAAAAAGCTTTCTGGAAGAAGACACCTTTCTGATAGAGGCTCAC
ACCTACAAATGCTTCACTCTGTCCTTCCGAGACCTGACAAGCTTTGAGGACCTCACAGCTCCCCTGT
GTGTTCATCTCTAGGGATGTTTGCAATTTCCCAGTCAGCTGTTCTGTCGCAGAATGTTTAATGCACAA
TTTTTTGCACTAGTGTGTTATGAATGACTAAGATTCTGATAAAAAAAATAAATTATTTACACAGGGTTT
ATACACACTATCCATTGTATATAAGCATTATTTCATATTATCAAGCTAAACATTCCCCCATCAGCTTAG
TTGGAGTGTTAGGGAAAAGTATTCCTAGATATGGCACAGATTTTAAAAGGAAATACAGTATTGAAGA
GATTTATTTTATTATTGCTTCAATTAGCTCCATTTACGTGTTGAATTCATTGAAGAGGTCCAATGAGAA
AAAAACAGAAGCCTCCTTATTTCACACGTTTTCCTCCTTTAGTACCATCCTCATCCAATTACTGTCTCT
CTGATACTACTTAATAGCAGGGGGTTTGCAGAAATTTCTGTTTGCCATGTAAAACTGTGAATAGTAAT
TTATTTTAGATAGTCGATGAACTTGTGGGTTTTAGCTCACAATGCAGCCTTCCCTTTTGCAGTGTTTTT
TTTTTGTTTTTTTTTTTTTTTGTCTTTTACTGTGCCATCGATCTTTGATATTGCATTGAAAGACAATATA
CCACAGTAGCACCTTGAACTCAGTGAAAATTGTTCAGGATCAAAATACCAAGTGTTCTTTTAGAGGGA
AGGAAAAAGTACACACACTCTCCTCTCACAATGATATATTTTATACATTCATTTGTTATTTGTTTCATG
CTTTATGATTCCAGATGGAAAGGTAATTTCAGTGACTTTTCAAGTTTAAATTCCATTATAGGTAAATGA
TAAGTTATGATGCAAATAAAATCTATAAGATCCCCAGGGCAAATAAAAATCAAAACATGAAGTAGAAG
ATGTGGCCGTGAGGTAGTTTATGTAACAAATTCAAAGTGAAAATCATGTTTACTTTTACTTATACTTAT
TTGATAAAAATATTTTTGAAACGATAGTACTTATTTTATTATTTGATATTTCAGTTCCTATTCAATTGTG
GCAGATTTTCTCTGTTTCACATTTTAGATTGGCGTTGGTAATAGAAATGTCAGAATGTTCAAATTGGC
CTTCACGTTGTCGGAGTGAACACATTGACACCTAGCTTTAAGACTGATTTATCTGTTGGTGTACTGAA
GGTTTCCATGTAGGACTTCAAATGTGGAAAAGGAAAAGCAGTCAGGAAAATGGGGCATTCTTTGGAG
AGTCACGCGTTTTGATTCGGACATTTCCGTAGAGCTCGGCTCCCAGTGTTGTGTTCCTCGGTCGAAA
GGGTCTCTGCTGTTTGGGGACTCACTGGCCTCTCCTAGGGACTCCTTTGTCTTGTGAACCCCACGCT
GTTGGATTCTGTATCATTATGCTGAATTCTCTGCACAGTTTTCCCTGGCCAACCTGCCCACATCCTTG
GAGATTTGCTTTGCCAGTGGGAATCCTTACATTGCTGTTTCACAGTAGACGGGACGAGGTCAGCGG
GAGTCGTGCTCCTAACACACACATTGAACGAAACAGAAGATGATTGAAAGTGTGAGGAGGCTCGTG
TGCAAGGGAGAACAGGGTTACTATACATATTAGTGTATATATATACATACATATATATATATATATATT
GTACATATCTAAGTTTGAGTCATTCAAACTAGGTGCAAAATGCTGACTTCAGAGTCTGAATTAACATC
TCTGTTCCCATATCCCTGACCTGCTCCCTGGTCAACGATGCTATGAAATCCTGAAATGACAGGACATA
CATACATACAAGAAACCACATATCAAATTAGATATGATTTTCCTTTGTGTGCAAAGTCAAACTGTCCTA
GGGTTGCCAGTTTGAAGCATGTTATTTAAATGAAAAAAAAAATCAGTGAAATTCTCGTGTGAGAATTC
TGCCTAGTTTCTTCCTAAGGTTGTGTGCAGTGTTGAACGGCGTCTCCGCAAGGTGTTGGAGGATCTC
ATTTTAGGGCAGTCAGGAGCTGTGCTTGCTGAGTTAGGTCTAGAAGACTCTTCCCTGAAGGCAACGG
GAACACGCGTGAGGGACGCGACCACACACTAACAGAGGACACGTGCTTCAGAGCTGTTTAAAACTG
CTGCTTGTTTTACACACACATCTTGCCTTTTTTCAGGCTAGCTGCAATAATTTTTTTCTTCTGTAAAATA
TTTTGTAAACAACAACAAAAAGCTATTATAAAAAGGGGGTAAAAAAAAGAACGCTGGCATTATGATCA
GGAAAACCCATTGTCATCGCCGACCCTCCCTCCCGTCCCACCACACGCTGCTGTCACGACGTAGGTG
CGAAAGACCTTTTTGTACAGAGATATATTTTTTATGAAGAATTTGTAAAATTATTAAATATGCTGTAAT
TTTTTGATTAATGTAGGTACATTGTTAAAAAATAAATGTTTTTACAATACAGAACTGTAATTTTCCCAAT
AATGTAAAATGTACCATCTCTAGCTGATTTTCAGTTCCAATCCTATTACACATGTATTAATATTAAAGT
GGCCTGTTAAAATGAACAGTATCTTTTTTTTGTCAAAAAAATTATAAAGAGGGTGTAATATAGCCTGT
GCAATGCCACCAATCTTTAAAGCAAATCAGAGTTCTAATTAAATATTTAATTTTA

### PTN

SEQ ID NO: 146: PTN-NRG1 fusion sequence
CCAGAACTGGAAGTCTGAAGCGAGCCCTGCACAATGCCGAATGCCAGAAG
ACTGTCACCATCTCCAAGCCCTGTGGCAAACTGACCAAGCCCAAACCTCA
AAGCCTTGCCTCCCCGATTGAAAGAGATGAAAAGCCAGGAATCGGCTGCA
GGTTCCAAACTAGTCCTTCGGTGTGAAACCAGTTCTGAATACTCCTCTCT
CAGATT

PTN sequence information, providing nucleotide sequence of exons 1-5 below as indicated by alternating underlining, starting with exon 1 underlined, exon 2 not underlined, exon 3 underlined, exon 4 not underlined, and so forth.
SEQ ID NO: 147=Exon 1 PTN
SEQ ID NO: 148=Exon 2 PTN
SEQ ID NO: 149=Exon 3 PTN
SEQ ID NO: 150=Exon 4 PTN
SEQ ID NO: 151=Exon 5 PTN
SEQ ID NO: 152=Exon 1-5 PTN
SEQ ID NO: 153=Exon 1-4 PTN

SEQ ID NO: 152
AAGGGGAAATAAGGGACAAGAGAGACCCTCTCATATTGTTTTATATTATT
TCATACTCAGAAAAGGAAAGAGAAGCCAAACAAAAGGCAGGTAACCCAGC
GCCTAGGAACCAGACCCGAAACCAAGGAACCAGATCTGAAACCAGGCCTG
GGCCTGCCTGACCTAAGCCTGGTAGTAAAAATTCCACCCCTGACCTGACC
TGGCAACTGTTGTTATCTACAGATTCCAGACATTGTATGGAAGGACACTG
TGAAACCTCCCGTTCTGTTCTGTTTCACTCTGACCATCGGTGCTCACAGC
CCCTATCACGTACCCCCTGGCTTGCTCAGTCGATCACGACCCTCTCACGT
GGACCCCCTTAGAGTTGTTAGCCCTTAAAAGGGACAGAAGTTGAGCACCT
GAGGAGCTCAGATTTTAAGACGCTAGGCTGCTGATGCTCCCAGCTGATTA
AAGCCACTCCCTTCACTATCTCGGTGTCTCCTGTCCGCGGCTCGTCCTGC
TACATTTCTTGGTTCCCTGACCGGCAAGCGAGAATGCAGGCTCAACAGTA
CCAGCAGCAGCGTCGAAAATTTGCAGCTGCCTTCTTGGCATTCATTTTCA
TACTGGCAGCTGTGGATACTGCTGAAGCAGGGAAGAAAGAGAAACCAGAA
AAAAAAGTGAAGAAGTCTGACTGTGGAGAATGGCAGTGGAGTGTGTGTGT
GCCCACCAGTGGAGACTGTGGGCTGGGCACACGGGAGGGCACTCGGACTG
GAGCTGAGTGCAAGCAAACCATGAAGACCCAGAGATGTAAGATCCCCTGC
AACTGGAAGAAGCAATTTGGCGCGGAGTGCAAATACCAGTTCCAGGCCTG
GGGAGAATGTGACCTGAACACAGCCCTGAAGACCAGAACTGGAAGTCTGA
AGCGAGCCCTGCACAATGCCGAATGCCAGAAGACTGTCACCATCTCCAAG
CCCTGTGGCAAACTGACCAAGCCCAAACCTCAAGCAGAATCTAAGAAGAA
GAAAAAGGAAGGCAAGAAACAGGAGAAGATGCTGGATTAAAAGATGTCAC
CTGTGGAACATAAAAAGGACATCAGCAAACAGGATCAGTTAACTATTGCA
TTTATATGTACCGTAGGCTTTGTATTCAAAAATTATCTATAGCTAAGTAC
ACAATAAGCAAAAACAAAAAGAAAAGAAAATTTTTGTAGTAGCGTTTTTT
AAATGTATACTATAGTACCAGTAGGGGCTTATAATAAAGGACTGTAATCT
TATTTAGGAAGTTGACTTATAGTACATGATAAATGATAGACAATTGAGGT
AAGTTTTTTGAAATTATGTGACATTTTACATTAAATTTTTTTTACATTTT
TTGGGCAGCAATTTAAATGTTATGACTATGTAAACTACTTCTCTTGTTAG
GTAATTTTTTTCACCTAGATTTTTTTCCCAATTGAGAAAAATATATACTA
AACAAAATAGCAATAAAACATAATCACTCTATTTGAAGAAAATATCTTGT
TTTCTGCCAATAGATTTTTTAAAATGTAGTCAGCAAAATGGGGGTGGGGA
AGCAGAGCATGTCCTAGTTCAATGTTGACTTTTTTTTTTTTAAAGAAAAG
CATTAAGACATAAAATTCTTTCACTTTGGCAGAAGCATTTGTTTTCTTGA
TGAAATTATTTTTCCATCTGAGGAAAAAAATACTAGGAAAATAAATCAAG
GTGATGCTGAAAAAAAAAAAAAAAAAAAAAAAAAAAAAA

### ST14

ST14-NRG1 fusion sequence
SEQ ID NO: 154
CAACAGCAACAAGATCACAGTTCGCTTCCACTCAGATCAGTCCTACACCG
ACACCGGCTTCTTAGCTGAATACCTCTCCTACGACTCCAGTGACCCTACA
TCTACATCCACCACTGGGACAAGCCATCTTGTAAAATGTGCGGAGAAGGA
GAAAACTTTCTGTGTGAATGGAGGGGAGTGCT

ST14 sequence information, providing nucleotide sequence of exons 1-19 below as indicated by alternating underlining, starting with exon 1 underlined, exon 2 not underlined, exon 3 underlined, exon 4 not underlined, and so forth.
SEQ ID NO: 155=Exon 1 ST14
SEQ ID NO: 156=Exon 2 ST14
SEQ ID NO: 157=Exon 3 ST14
SEQ ID NO: 158=Exon 4 ST14
SEQ ID NO: 159=Exon 5 ST14
SEQ ID NO: 160=Exon 6 ST14
SEQ ID NO: 161=Exon 7 ST14
SEQ ID NO: 162=Exon 8 ST14
SEQ ID NO: 163=Exon 9 ST14
SEQ ID NO: 164=Exon 10 ST14
SEQ ID NO: 165=Exon 11 ST14
SEQ ID NO: 166=Exon 12 ST14
SEQ ID NO: 167=Exon 13 ST14
SEQ ID NO: 168=Exon 14 ST14
SEQ ID NO: 169=Exon 15 ST14
SEQ ID NO: 170=Exon 16 ST14
SEQ ID NO: 171=Exon 17 ST14
SEQ ID NO: 172=Exon 18 ST14
SEQ ID NO: 173=Exon 19 ST14
SEQ ID NO: 174=Exon 1-19 ST14
SEQ ID NO: 175=Exon 1-11 ST14

SEQ ID NO: 174
GTGAGAGCGGAGCTGCAGCCGGAGAAAGAGGAAGAGGGAGAGAGAGCGCG
CCAGGGCGAGGGCACCGCCGCCGGTCGGGCGCGCTGGGCCTGCCCGGAAT
CCCGCCGCCTGCGCCCCGCGCCCCGCGCCCTGCGGGCCATGGGAGCCGGC
CGCCGGCAGGGACGACGCCTGTGAGACCCGCGAGCGGCCTCGGGGACCAT
GGGGAGCGATCGGGCCCGCAAGGGCGGAGGGGGCCCGAAGGACTTCGGCG
CGGGACTCAAGTACAACTCCCGGCACGAGAAAGTGAATGGCTTGGAGGAA
GGCGTGGAGTTCCTGCCAGTCAACAACGTCAAGAAGGTGGAAAAGCATGG
CCCGGGGCGCTGGGTGGTGCTGGCAGCCGTGCTGATCGGCCTCCTCTTGG
TCTTGCTGGGGATCGGCTTCCTGGTGTGGCATTTGCAGTACCGGGACGTG
CGTGTCCAGAAGGTCTTCAATGGCTACATGAGGATCACAAATGAGAATTT
TGTGGATGCCTACGAGAACTCCAACTCCACTGAGTTTGTAAGCCTGGCCA
GCAAGGTGAAGGACGCGCTGAAGCTGCTGTACAGCGGAGTCCCATTCCTG
GGCCCCTACCACAAGGAGTCGGCTGTGACGGCCTTCAGCGAGGGCAGCGT
CATCGCCTACTACTGGTCTGAGTTCAGCATCCCGCAGCACCTGGTGGAGG
AGGCCGAGCGCGTCATGGCCGAGGAGCGCGTAGTCATGCTGCCCCCGCGG
GCGCGCTCCCTGAAGTCCTTTGTGGTCACCTCAGTGGTGGCTTTCCCCAC
GGACTCCAAAACAGTACAGAGGACCCAGGACAACAGCTGCAGCTTTGGCC
TGCACGCCCGCGGTGTGGAGCTGATGCGCTTCACCACGCCCGGCTTCCCT
GACAGCCCCTACCCCGCTCATGCCCGCTGCCAGTGGGCCCTGCGGGGGGA
CGCCGACTCAGTGCTGAGCCTCACCTTCCGCAGCTTTGACCTTGCGTCCT
GCGACGAGCGCGGCAGCGACCTGGTGACGGTGTACAACACCCTGAGCCCC
ATGGAGCCCCACGCCCTGGTGCAGTTGTGTGGCACCTACCCTCCCTCCTA
CAACCTGACCTTCCACTCCTCCCAGAACGTCCTGCTCATCACACTGATAA
CCAACACTGAGCGGCGGCATCCCGGCTTTGAGGCCACCTTCTTCCAGCTG
CCTAGGATGAGCAGCTGTGGAGGCCGCTTACGTAAAGCCCAGGGGACATT
CAACAGCCCCTACTACCCAGGCCACTACCCACCCAACATTGACTGCACAT
GGAACATTGAGGTGCCCAACAACCAGCATGTGAAGGTGCGCTTCAAATTC
TTCTACCTGCTGGAGCCCGGCGTGCCTGCGGGCACCTGCCCCAAGGACTA
CGTGGAGATCAACGGGGAGAAATACTGCGGAGAGAGGTCCCAGTTCGTCG
TCACCAGCAACAGCAACAAGATCACAGTTCGCTTCCACTCAGATCAGTCC
TACACCGACACCGGCTTCTTAGCTGAATACCTCTCCTACGACTCCAGTGA
CCCATGCCCGGGGCAGTTCACGTGCCGCACGGGGCGGTGTATCCGGAAGG
AGCTGCGCTGTGATGGCTGGGCCGACTGCACCGACCACAGCGATGAGCTC
AACTGCAGTTGCGACGCCGGCCACCAGTTCACGTGCAAGAACAAGTTCTG
CAAGCCCCTCTTCTGGGTCTGCGACAGTGTGAACGACTGCGGAGACAACA
GCGACGAGCAGGGGTGCAGTTGTCCGGCCCAGACCTTCAGGTGTTCCAAT
GGGAAGTGCCTCTCGAAAAGCCAGCAGTGCAATGGGAAGGACGACTGTGG
GGACGGGTCCGACGAGGCCTCCTGCCCCAAGGTGAACGTCGTCACTTGTA
CCAAACACACCTACCGCTGCCTCAATGGGCTCTGCTTGAGCAAGGGCAAC
CCTGAGTGTGACGGGAAGGAGGACTGTAGCGACGGCTCAGATGAGAAGGA
CTGCGACTGTGGGCTGCGGTCATTCACGAGACAGGCTCGTGTTGTTGGGG
GCACGGATGCGGATGAGGGCGAGTGGCCCTGGCAGGTAAGCCTGCATGCT
CTGGGCCAGGGCCACATCTGCGGTGCTTCCCTCATCTCTCCCAACTGGCT
GGTCTCTGCCGCACACTGCTACATCGATGACAGAGGATTCAGGTACTCAG
ACCCCACGCAGTGGACGGCCTTCCTGGGCTTGCACGACCAGAGCCAGCGC
AGCGCCCCTGGGGTGCAGGAGCGCAGGCTCAAGCGCATCATCTCCCACCC
CTTCTTCAATGACTTCACCTTCGACTATGACATCGCGCTGCTGGAGCTGG
AGAAACCGGCAGAGTACAGCTCCATGGTGCGGCCCATCTGCCTGCCGGAC
GCCTCCCATGTCTTCCCTGCCGGCAAGGCCATCTGGGTCACGGGCTGGGG
ACACACCCAGTATGGAGGCACTGGCGCGCTGATCCTGCAAAAGGGTGAGA
TCCGCGTCATCAACCAGACCACCTGCGAGAACCTCCTGCCGCAGCAGATC
ACGCCGCGCATGATGTGCGTGGGCTTCCTCAGCGGCGGCGTGGACTCCTG
CCAGGGTGATTCCGGGGGACCCCTGTCCAGCGTGGAGGCGGATGGGCGGA
TCTTCCAGGCCGGTGTGGTGAGCTGGGGAGACGGCTGCGCTCAGAGGAAC
AAGCCAGGCGTGTACACAAGGCTCCCTCTGTTTCGGGACTGGATCAAAGA
GAACACTGGGGTATAGGGGCCGGGGCCACCCAAATGTGTACACCTGCGGG
GCCACCCATCGTCCACCCCAGTGTGCACGCCTGCAGGCTGGAGACTGGAC
CGCTGACTGCACCAGCGCCCCCAGAACATACACTGTGAACTCAATCTCCA
GGGCTCCAAATCTGCCTAGAAAACCTCTCGCTTCCTCAGCCTCCAAAGTG
GAGCTGGGAGGTAGAAGGGGAGGACACTGGTGGTTCTACTGACCCAACTG
GGGGCAAAGGTTTGAAGACACAGCCTCCCCCGCCAGCCCCAAGCTGGGCC
GAGGCGCGTTTGTGCATATCTGCCTCCCCTGTCTCTAAGGAGCAGCGGGA
ACGGAGCTTCGGGGCCTCCTCAGTGAAGGTGGTGGGGCTGCCGGATCTGG
GCTGTGGGGCCCTTGGGCCACGCTCTTGAGGAAGCCCAGGCTCGGAGGAC
CCTGGAAAACAGACGGGTCTGAGACTGAAATTGTTTTACCAGCTCCCAGG
GTGGACTTCAGTGTGTGTATTTGTGTAAATGAGTAAAACATTTTATTTCT
TTTTA

### THBS1

THBS1-NRG1 fusion sequence
SEQ ID NO: 176
ACCCTGTGAAGGCGAAGCGCGGGAGACCAAAGCCTGCAAGAAAGACGCCT
GCCCCACTACATCTACATCCACCACTGGGACAAGCCATCTTGTAAAATGT
GCGGAGAAGGAGAAAACTTTCTGTGTGAATGGAGGGGAGTGCTTC

THBS1 sequence information, providing nucleotide sequence of exons 1-22 below as indicated by alternating underlining, starting with exon 1 underlined, exon 2 not underlined, exon 3 underlined, exon 4 not underlined, and so forth.
SEQ ID NO: 177=Exon 1 THBS1
SEQ ID NO: 178=Exon 2 THBS1
SEQ ID NO: 179=Exon 3 THBS1
SEQ ID NO: 180=Exon 4 THBS1
SEQ ID NO: 181=Exon 5 THBS1
SEQ ID NO: 182=Exon 6 THBS1
SEQ ID NO: 183=Exon 7 THBS1
SEQ ID NO: 184=Exon 8 THBS1
SEQ ID NO: 185=Exon 9 THBS1
SEQ ID NO: 186=Exon 10 THBS1
SEQ ID NO: 187=Exon 11 THBS1
SEQ ID NO: 188=Exon 12 THBS1
SEQ ID NO: 189=Exon 13 THBS1
SEQ ID NO: 190=Exon 14 THBS1
SEQ ID NO: 191=Exon 15 THBS1
SEQ ID NO: 192=Exon 16 THBS1
SEQ ID NO: 193=Exon 17 THBS1
SEQ ID NO: 194=Exon 18 THBS1
SEQ ID NO: 195=Exon 19 THBS1
SEQ ID NO: 196=Exon 20 THBS1
SEQ ID NO: 197=Exon 21 THBS1
SEQ ID NO: 198=Exon 22 THBS1
SEQ ID NO: 199=Exon 1-22 THBS1
SEQ ID NO: 200=Exon 1-9 THBS1

SEQ ID NO: 199
AGCCGCTGCGCCCGAGCTGGCCTGCGAGTTCAGGGCTCCTGTCGCTCTCCAGGAGCAACCTCTACT
CCGGACGCACAGGCATTCCCCGCGCCCCTCCAGCCCTCGCCGCCCTCGCCACCGCTCCCGGCCGCC
GCGCTCCGGTACACACAGGATCCCTGCTGGGCACCAACAGCTCCACCATGGGGCTGGCCTGGGGA
CTAGGCGTCCTGTTCCTGATGCATGTGTGTGGCACCAACCGCATTCCAGAGTCTGGCGGAGACAAC
AGCGTGTTTGACATCTTTGAACTCACCGGGGCCGCCCGCAAGGGGTCTGGGCGCCGACTGGTGAAG
GGCCCCGACCCTTCCAGCCCAGCTTTCCGCATCGAGGATGCCAACCTGATCCCCCCTGTGCCTGATG
ACAAGTTCCAAGACCTGGTGGATGCTGTGCGGGCAGAAAAGGGTTTCCTCCTTCTGGCATCCCTGA
GGCAGATGAAGAAGACCCGGGGCACGCTGCTGGCCCTGGAGCGGAAAGACCACTCTGGCCAGGTC
TTCAGCGTGGTGTCCAATGGCAAGGCGGGCACCCTGGACCTCAGCCTGACCGTCCAAGGAAAGCAG
CACGTGGTGTCTGTGGAAGAAGCTCTCCTGGCAACCGGCCAGTGGAAGAGCATCACCCTGTTTGTG
CAGGAAGACAGGGCCCAGCTGTACATCGACTGTGAAAAGATGGAGAATGCTGAGTTGGACGTCCCC
ATCCAAAGCGTCTTCACCAGAGACCTGGCCAGCATCGCCAGACTCCGCATCGCAAAGGGGGGCGTC
AATGACAATTTCCAGGGGGTGCTGCAGAATGTGAGGTTTGTCTTTGGAACCACACCAGAAGACATCC
TCAGGAACAAAGGCTGCTCCAGCTCTACCAGTGTCCTCCTCACCCTTGACAACAACGTGGTGAATGG
TTCCAGCCCTGCCATCCGCACTAACTACATTGGCCACAAGACAAAGGACTTGCAAGCCATCTGCGGC
ATCTCCTGTGATGAGCTGTCCAGCATGGTCCTGGAACTCAGGGGCCTGCGCACCATTGTGACCACG
CTGCAGGACAGCATCCGCAAAGTGACTGAAGAGAACAAAGAGTTGGCCAATGAGCTGAGGCGGCCT
CCCCTATGCTATCACAACGGAGTTCAGTACAGAAATAACGAGGAATGGACTGTTGATAGCTGCACTG
AGTGTCACTGTCAGAACTCAGTTACCATCTGCAAAAAGGTGTCCTGCCCCATCATGCCCTGCTCCAAT
GCCACAGTTCCTGATGGAGAATGCTGTCCTCGCTGTTGGCCCAGCGACTCTGCGGACGATGGCTGG
TCTCCATGGTCCGAGTGGACCTCCTGTTCTACGAGCTGTGGCAATGGAATTCAGCAGCGCGGCCGC
TCCTGCGATAGCCTCAACAACCGATGTGAGGGCTCCTCGGTCCAGACACGGACCTGCCACATTCAG
GAGTGTGACAAGAGATTTAAACAGGATGGTGGCTGGAGCCACTGGTCCCCGTGGTCATCTTGTTCT
GTGACATGTGGTGATGGTGTGATCACAAGGATCCGGCTCTGCAACTCTCCCAGCCCCCAGATGAAC
GGGAAACCCTGTGAAGGCGAAGCGCGGGAGACCAAAGCCTGCAAGAAAGACGCCTGCCCCATCAA
TGGAGGCTGGGGTCCTTGGTCACCATGGGACATCTGTTCTGTCACCTGTGGAGGAGGGGTACAGAA
ACGTAGTCGTCTCTGCAACAACCCCACACCCCAGTTTGGAGGCAAGGACTGCGTTGGTGATGTAACA
GAAAACCAGATCTGCAACAAGCAGGACTGTCCAATTGATGGATGCCTGTCCAATCCCTGCTTTGCCG
GCGTGAAGTGTACTAGCTACCCTGATGGCAGCTGGAAATGTGGTGCTTGTCCCCCTGGTTACAGTG
GAAATGGCATCCAGTGCACAGATGTTGATGAGTGCAAAGAAGTGCCTGATGCCTGCTTCAACCACAA
TGGAGAGCACCGGTGTGAGAACACGGACCCCGGCTACAACTGCCTGCCCTGCCCCCCACGCTTCAC
CGGCTCACAGCCCTTCGGCCAGGGTGTCGAACATGCCACGGCCAACAAACAGGTGTGCAAGCCCCG
TAACCCCTGCACGGATGGGACCCACGACTGCAACAAGAACGCCAAGTGCAACTACCTGGGCCACTA
TAGCGACCCCATGTACCGCTGCGAGTGCAAGCCTGGCTACGCTGGCAATGGCATCATCTGCGGGGA
GGACACAGACCTGGATGGCTGGCCCAATGAGAACCTGGTGTGCGTGGCCAATGCGACTTACCACTG
CAAAAAGGATAATTGCCCCAACCTTCCCAACTCAGGGCAGGAAGACTATGACAAGGATGGAATTGGT
GATGCCTGTGATGATGACGATGACAATGATAAAATTCCAGATGACAGGGACAACTGTCCATTCCATT
ACAACCCAGCTCAGTATGACTATGACAGAGATGATGTGGGAGACCGCTGTGACAACTGTCCCTACAA
CCACAACCCAGATCAGGCAGACACAGACAACAATGGGGAAGGAGACGCCTGTGCTGCAGACATTGA
TGGAGACGGTATCCTCAATGAACGGGACAACTGCCAGTACGTCTACAATGTGGACCAGAGAGACAC
TGATATGGATGGGGTTGGAGATCAGTGTGACAATTGCCCCTTGGAACACAATCCGGATCAGCTGGA
CTCTGACTCAGACCGCATTGGAGATACCTGTGACAACAATCAGGATATTGATGAAGATGGCCACCAG
AACAATCTGGACAACTGTCCCTATGTGCCCAATGCCAACCAGGCTGACCATGACAAAGATGGCAAGG
GAGATGCCTGTGACCACGATGATGACAACGATGGCATTCCTGATGACAAGGACAACTGCAGACTCG
TGCCCAATCCCGACCAGAAGGACTCTGACGGCGATGGTCGAGGTGATGCCTGCAAAGATGATTTTG
ACCATGACAGTGTGCCAGACATCGATGACATCTGTCCTGAGAATGTTGACATCAGTGAGACCGATTT
CCGCCGATTCCAGATGATTCCTCTGGACCCCAAAGGGACATCCCAAAATGACCCTAACTGGGTTGTA
CGCCATCAGGGTAAAGAACTCGTCCAGACTGTCAACTGTGATCCTGGACTCGCTGTAGGTTATGATG
AGTTTAATGCTGTGGACTTCAGTGGCACCTTCTTCATCAACACCGAAAGGGACGATGACTATGCTGG
ATTTGTCTTTGGCTACCAGTCCAGCAGCCGCTTTTATGTTGTGATGTGGAAGCAAGTCACCCAGTCCT
ACTGGGACACCAACCCCACGAGGGCTCAGGGATACTCGGGCCTTTCTGTGAAAGTTGTAAACTCCA
CCACAGGGCCTGGCGAGCACCTGCGGAACGCCCTGTGGCACACAGGAAACACCCCTGGCCAGGTG
CGCACCCTGTGGCATGACCCTCGTCACATAGGCTGGAAAGATTTCACCGCCTACAGATGGCGTCTCA
GCCACAGGCCAAAGACGGGTTTCATTAGAGTGGTGATGTATGAAGGGAAGAAAATCATGGCTGACT
CAGGACCCATCTATGATAAAACCTATGCTGGTGGTAGACTAGGGTTGTTTGTCTTCTCTCAAGAAATG
GTGTTCTTCTCTGACCTGAAATACGAATGTAGAGATCCCTAATCATCAAATTGTTGATTGAAAGACTG
ATCATAAACCAATGCTGGTATTGCACCTTCTGGAACTATGGGCTTGAGAAAACCCCCAGGATCACTT
CTCCTTGGCTTCCTTCTTTTCTGTGCTTGCATCAGTGTGGACTCCTAGAACGTGCGACCTGCCTCAAG
AAAATGCAGTTTTCAAAAACAGACTCAGCATTCAGCCTCCAATGAATAAGACATCTTCCAAGCATATA
AACAATTGCTTTGGTTTCCTTTTGAAAAAGCATCTACTTGCTTCAGTTGGGAAGGTGCCCATTCCACT
CTGCCTTTGTCACAGAGCAGGGTGCTATTGTGAGGCCATCTCTGAGCAGTGGACTCAAAAGCATTTT
CAGGCATGTCAGAGAAGGGAGGACTCACTAGAATTAGCAAACAAAACCACCCTGACATCCTCCTTCA
GGAACACGGGGAGCAGAGGCCAAAGCACTAAGGGGAGGGCGCATACCCGAGACGATTGTATGAAG
AAAATATGGAGGAACTGTTACATGTTCGGTACTAAGTCATTTTCAGGGGATTGAAAGACTATTGCTG
GATTTCATGATGCTGACTGGCGTTAGCTGATTAACCCATGTAAATAGGCACTTAAATAGAAGCAGGA
AAGGGAGACAAAGACTGGCTTCTGGACTTCCTCCCTGATCCCCACCCTTACTCATCACCTGCAGTGG
CCAGAATTAGGGAATCAGAATCAAACCAGTGTAAGGCAGTGCTGGCTGCCATTGCCTGGTCACATTG
AAATTGGTGGCTTCATTCTAGATGTAGCTTGTGCAGATGTAGCAGGAAAATAGGAAAACCTACCATC
TCAGTGAGCACCAGCTGCCTCCCAAAGGAGGGGCAGCCGTGCTTATATTTTTATGGTTACAATGGCA
CAAAATTATTATCAACCTAACTAAAACATTCCTTTTCTCTTTTTTCCTGAATTATCATGGAGTTTTCTAA
TTCTCTCTTTTGGAATGTAGATTTTTTTTAAATGCTTTACGATGTAAAATATTTATTTTTTACTTATTCTG
GAAGATCTGGCTGAAGGATTATTCATGGAACAGGAAGAAGCGTAAAGACTATCCATGTCATCTTTGT
TGAGAGTCTTCGTGACTGTAAGATTGTAAATACAGATTATTTATTAACTCTGTTCTGCCTGGAAATTTA
GGCTTCATACGGAAAGTGTTTGAGAGCAAGTAGTTGACATTTATCAGCAAATCTCTTGCAAGAACAG
CACAAGGAAAATCAGTCTAATAAGCTGCTCTGCCCCTTGTGCTCAGAGTGGATGTTATGGGATTCTTT
TTTTCTCTGTTTTATCTTTTCAAGTGGAATTAGTTGGTTATCCATTTGCAAATGTTTTAAATTGCAAAGA
AAGCCATGAGGTCTTCAATACTGTTTTACCCCATCCCTTGTGCATATTTCCAGGGAGAAGGAAAGCAT
ATACACTTTTTTCTTTCATTTTTCCAAAAGAGAAAAAAATGACAAAAGGTGAAACTTACATACAAATAT
TACCTCATTTGTTGTGTGACTGAGTAAAGAATTTTTGGATCAAGCGGAAAGAGTTTAAGTGTCTAACA
AACTTAAAGCTACTGTAGTACCTAAAAAGTCAGTGTTGTACATAGCATAAAAACTCTGCAGAGAAGTA
TTCCCAATAAGGAAATAGCATTGAAATGTTAAATACAATTTCTGAAAGTTATGTTTTTTTTCTATCATCT
GGTATACCATTGCTTTATTTTTATAAATTATTTTCTCATTGCCATTGGAATAGATATCTCAGATTGTGTA
GATATGCTATTTAAATAATTTATCAGGAAATACTGCCTGTAGAGTTAGTATTTCTATTTTTATATAATGT
TTGCACACTGAATTGAAGAATTGTTGGTTTTTTCTTTTTTTTGTTTTGTTTTTTTTTTTTTTTTTTTTTGC
TTTTGACCTCCCATTTTTACTATTTGCCAATACCTTTTTCTAGGAATGTGCTTTTTTTTGTACACATTTT
TATCCATTTTACATTCTAAAGCAGTGTAAGTTGTATATTACTGTTTCTTATGTACAAGGAACAACAATA
AATCATATGGAAATTTATATTTATACTTACTGTATCCATGCTTATTTGTTCTCTACTGGCTTTATGTCAT
GAAGTATATGCGTAAATACCATTCATAAATCAATATAGCATATACAAAAATAAATTACAGTAAGTCATA
GCAACATTCACAGTTTGTATGTGATTGAGAAAGACTGAGTTGCTCAGGCCTAGGCTTAGAATTTGCT
GCGTTTGTGGAATAAAAGAACAAAATGATACATTAGCCTGCCATATCAAAAACATATAAAAGAGAAAT
TATCCCTAAGTCAAGGGCCCCCATAAGAATAAAATTTCTTATTAAGGTCATTAGATGTCATTGAATCC
TTTTCAAAGTGCAGTATGAAAACAAAGGGAAAAACACTGAAGCACACGCAACTCTCACAGCGACATT
TTCTGACCCACGAATGATGCCTTGGGTGGGCAACACGATTGCATGTTGTGGAGACACTTCGGAAGTA
AATGTGGATGAGGGAGGAGCTGTCCTTGCAATGTTGAGCCAAGCATTACAGATACCTCCTCTTGAAG
AAGGAATAATAAGTTTAATCAAAAAAGAAGACTAAAAAATGTAAAATTTGGAAGGAATCCATAAATGC
GTGTGTGTCTAAATACAAATTATCATGTGAAGAAAAGGCCCAAGTGTACCAATAAGCAGACCTTGATT
TTTGGATGGGCTAATTATGAATGTGGAATACTGACCAGTTAATTTCCAGTTTTAATGAAAACAGATCA
AAGAAGAAATTTTATGAGTAGGTTAAAGGTCTGGCTTTGAGGTCTATTAAACACTAGAAAGGACTGG
CTGGGTGAGATAAAATCTTCCTTGTTGATTTTCACTCTCATTCTATAAATACTCATCTTTCTGAGTAGC
CATGATCACATACAAATGTAAATTGCCAAATCATTTTATAGTACCAAGGTGAAGAAGCAGGAACTAGA
AAGTGTTGATAATAGCTGTGGAGTTAGGAAAACTGATGTGAAGGAAATAATTCTTTGAAATGGCAAA
GAATTAAATACCATCATTCATTATCAGAAGAGTTCAACGTTTGAAGTGCTGGGAGATAATTCTAATTC
ATTCTTGGATAGTGAAGCAAAACTGATTGAAAATACCAAGATAAGACAGAAAAAGTGACTGGAAAGA
GGAGCTTTTCTTCCAGGCATGTTCCAGTTTCACCCTAAGACTGACCTTCAAATAATCAGGTTGTACTG
AAATAAAGGACTTGTTAAAAATTAAAATTATGTCATCGAGATGATAGCTTTTTTCCTCCTCCAACAGTT
TATTGTCATGTGTTGTGGGAGAGCTCGAGTGAAGAGCAATAAACTCCAGGTCTTATAAGAATGTACA
TACAATAAAGGTGGTGCCAGCAGTTTTTTTTTTTCTAAAGAGTCACATGTAGAAAAGCCTCCAGTATT
AAGCTCCTGAATTCATTCCTTAAATAAATTGGCTCTCTCTCTCTTCTATAATTTCTTTTTCTTTTTATTTT
TGAGATGAAGTCTTGCTCTGTCGCCCAGGCTGGAGTGCAGTGACACAATCTCGGCTCACTGCAACCT
CTGCCTCCCCGGTTCAAGCAATTCTCCCTCCTGCCTCAGCCTCCCAAGTAGCTGGGACTACAAGCGC
CCGCCACCAAGCCTGGCTAATTCTGTATTTTTAGTAAAGACGGGGTTTCACCTTGTTCCGGACAAACA
CTAAGCCCTAAAGGGAAATCCAAAATAAAAACATCTATTTTTAATAACACTTTCTATCTAAATCAGGGT
GACTTTTTAAAAAAAATCCGGAAGCTTTTTGTTGAATTACGTTACAGACTTAGTTACCAGTCCTTGTTA
GAGTTACCTTCAGTTGACATGCTGTGAATGGTCCCACCTCTTTTATGGCAGAATTCATTACTTAAAAT
AACTCTATTTTCTTCCCCCTTACCTAAATAACAGAAAGGCTCACTATGTCCCAAATATCATTGGCAGAA
GCAAACTATAAAGTCATAAGCCCTTTGCAGTGCAAGTCTAGAAATAATTTT

### AGRN

AGRN-NRG1 fusion sequence
SEQ ID NO: 201
GTGTGCGGCTCAGATGGGGTCACCTACAGCACCGAGTGTGAGCTG
AAGAAGGCCAGGTGTGAGTCACAGCGAGGGCTCTACGTAGCGGCC
CAGGGAGCCTGCCGAGCTACATCTACATCCACCACTGGGACAAGC
CATCTTGTAAAATGTGCGGAGAAGGAGAAAACTTTCTGTGTGAAT
GGAGGGGAGTGCTTCATGGTGAAAGAC

AGRN sequence information, providing nucleotide sequence of exons 1-39 below as indicated by alternating underlining, starting with exon 1 underlined, exon 2 not underlined, exon 3 underlined, exon 4 not underlined, and so forth up until and including exon 13.
SEQ ID NO: 202=Exon 1 AGRN
SEQ ID NO: 203=Exon 2 AGRN
SEQ ID NO: 204=Exon 3 AGRN
SEQ ID NO: 205=Exon 4 AGRN
SEQ ID NO: 206=Exon 5 AGRN
SEQ ID NO: 207=Exon 6 AGRN
SEQ ID NO: 208=Exon 7 AGRN
SEQ ID NO: 209=Exon 8 AGRN
SEQ ID NO: 210=Exon 9 AGRN
SEQ ID NO: 211=Exon 10 AGRN
SEQ ID NO: 212=Exon 11 AGRN
SEQ ID NO: 213=Exon 12 AGRN
SEQ ID NO: 214=Exon 13 AGRN
SEQ ID NO: 215=Exon 1-39 AGRN
SEQ ID NO: 216=Exon 1-12 AGRN

SEQ ID NO: 215
AGTCCCGTCCCCGGCGCGGCCCGCGCGCTCCTCCGCCGCCTCTCG
CCTGCGCCATGGCCGGCCGGTCCCACCCGGGCCCGCTGCGGCCGC
TGCTGCCGCTCCTTGTGGTGGCCGCGTGCGTCCTGCCCGGAGCCG
GCGGGACATGCCCGGAGCGCGCGCTGGAGCGGCGCGAGGAGGAGG
CGAACGTGGTGCTCACCGGGACGGTGGAGGAGATCCTCAACGTGG
ACCCGGTGCAGCACACGTACTCCTGCAAGGTTCGGGTCTGGCGGT
ACTTGAAGGGCAAAGACCTGGTGGCCCGGGAGAGCCTGCTGGACG
GCGGCAACAAGGTGGTGATCAGCGGCTTTGGAGACCCCCTCATCT
GTGACAACCAGGTGTCCACTGGGGACACCAGGATCTTCTTTGTGA
ACCCTGCACCCCCATACCTGTGGCCAGCCCACAAGAACGAGCTGA
TGCTCAACTCCAGCCTCATGCGGATCACCCTGCGGAACCTGGAGG
AGGTGGAGTTCTGTGTGGAAGATAAACCCGGGACCCACTTCACTC
CAGTGCCTCCGACGCCTCCTGATGCGTGCCGGGGAATGCTGTGCG
GCTTCGGCGCCGTGTGCGAGCCCAACGCGGAGGGGCCGGGCCGGG
CGTCCTGCGTCTGCAAGAAGAGCCCGTGCCCCAGCGTGGTGGCGC
CTGTGTGTGGGTCGGACGCCTCCACCTACAGCAACGAATGCGAGC
TGCAGCGGGCGCAGTGCAGCCAGCAGCGCCGCATCCGCCTGCTCA
GCCGCGGGCCGTGCGGCTCGCGGGACCCCTGCTCCAACGTGACCT
GCAGCTTCGGCAGCACCTGTGCGCGCTCGGCCGACGGGCTGACGG
CCTCGTGCCTGTGCCCCGCGACCTGCCGTGGCGCCCCCGAGGGGA
CCGTCTGCGGCAGCGACGGCGCCGACTACCCCGGCGAGTGCCAGC
TCCTGCGCCGCGCCTGCGCCCGCCAGGAGAATGTCTTCAAGAAGT
TCGACGGCCCTTGTGACCCCTGTCAGGGCGCCCTCCCTGACCCGA
GCCGCAGCTGCCGTGTGAACCCGCGCACGCGGCGCCCTGAGATGC
TCCTACGGCCCGAGAGCTGCCCTGCCCGGCAGGCGCCAGTGTGTG
GGGACGACGGAGTCACCTACGAAAACGACTGTGTCATGGGCCGAT
CGGGGGCCGCCCGGGGTCTCCTCCTGCAGAAAGTGCGCTCCGGCC
AGTGCCAGGGTCGAGACCAGTGCCCGGAGCCCTGCCGGTTCAATG
CCGTGTGCCTGTCCCGCCGTGGCCGTCCCCGCTGCTCCTGCGACC
GCGTCACCTGTGACGGGGCCTACAGGCCCGTGTGTGCCCAGGACG
GGCGCACGTATGACAGTGATTGCTGGCGGCAGCAGGCTGAGTGCC
GGCAGCAGCGTGCCATCCCCAGCAAGCACCAGGGCCCGTGTGACC
AGGCCCCGTCCCCATGCCTCGGGGTGCAGTGTGCATTTGGGGCGA
CGTGTGCTGTGAAGAACGGGCAGGCAGCGTGTGAATGCCTGCAGG
CGTGCTCGAGCCTCTACGATCCTGTGTGCGGCAGCGACGGCGTCA
CATACGGCAGCGCGTGCGAGCTGGAGGCCACGGCCTGTACCCTCG
GGCGGGAGATCCAGGTGGCGCGCAAAGGACCCTGTGACCGCTGCG
GGCAGTGCCGCTTTGGAGCCCTGTGCGAGGCCGAGACCGGGCGCT
GCGTGTGCCCCTCTGAATGCGTGGCTTTGGCCCAGCCCGTGTGTG
GCTCCGACGGGCACACGTACCCCAGCGAGTGCATGCTGCACGTGC
ACGCCTGCACACACCAGATCAGCCTGCACGTGGCCTCAGCTGGAC
CCTGTGAGACCTGTGGAGATGCCGTGTGTGCTTTTGGGGCTGTGT
GCTCCGCAGGGCAGTGTGTGTGTCCCCGGTGTGAGCACCCCCCGC
CCGGCCCCGTGTGTGGCAGCGACGGTGTCACCTACGGCAGTGCCT
GCGAGCTACGGGAAGCCGCCTGCCTCCAGCAGACACAGATCGAGG
AGGCCCGGGCAGGGCCGTGCGAGCAGGCCGAGTGCGGTTCCGGAG
GCTCTGGCTCTGGGGAGGACGGTGACTGTGAGCAGGAGCTGTGCC
GGCAGCGCGGTGGCATCTGGGACGAGGACTCGGAGGACGGGCCGT
GTGTCTGTGACTTCAGCTGCCAGAGTGTCCCAGGCAGCCCGGTGT
GCGGCTCAGATGGGGTCACCTACAGCACCGAGTGTGAGCTGAAGA
AGGCCAGGTGTGAGTCACAGCGAGGGCTCTACGTAGCGGCCCAGG
GAGCCTGCCGAGGCCCCACCTTCGCCCCGCTGCCGCCTGTGGCCC
CCTTACACTGTGCCCAGACGCCCTACGGCTGCTGCCAGGACAATA
TCACCGCAGCCCGGGGCGTGGGCCTGGCTGGCTGCCCCAGTGCCT
GCCAGTGCAACCCCCATGGCTCTTACGGCGGCACCTGTGACCCAG
CCACAGGCCAGTGCTCCTGCCGCCCAGGTGTGGGGGGCCTCAGGT
GTGACCGCTGTGAGCCTGGCTTCTGGAACTTTCGAGGCATCGTCA
CCGATGGCCGGAGTGGCTGTACACCCTGCAGCTGTGATCCCCAAG
GCGCCGTGCGGGATGACTGTGAGCAGATGACGGGGCTGTGCTCGT
GTAAGCCCGGGGTGGCTGGACCCAAGTGTGGGCAGTGTCCAGACG
GCCGTGCCCTGGGCCCCGCGGGCTGTGAAGCTGACGCTTCTGCGC
CTGCGACCTGTGCGGAGATGCGCTGTGAGTTCGGTGCGCGGTGCG
TGGAGGAGTCTGGCTCAGCCCACTGTGTCTGCCCGATGCTCACCT
GTCCAGAGGCCAACGCTACCAAGGTCTGTGGGTCAGATGGAGTCA
CATACGGCAACGAGTGTCAGCTGAAGACCATCGCCTGCCGCCAGG
GCCTGCAAATCTCTATCCAGAGCCTGGGCCCGTGCCAGGAGGCTG
TTGCTCCCAGCACTCACCCGACATCTGCCTCCGTGACTGTGACCA
CCCCAGGGCTCCTCCTGAGCCAGGCACTGCCGGCCCCCCCCGGCG
CCCTCCCCCTGGCTCCCAGCAGTACCGCACACAGCCAGACCACCC
CTCCGCCCTCATCACGACCTCGGACCACTGCCAGCGTCCCCAGGA
CCACCGTGTGGCCCGTGCTGACGGTGCCCCCCACGGCACCCTCCC
CTGCACCCAGCCTGGTGGCGTCCGCCTTTGGTGAATCTGGCAGCA
CTGATGGAAGCAGCGATGAGGAACTGAGCGGGGACCAGGAGGCCA
GTGGGGGTGGCTCTGGGGGGCTCGAGCCCTTGGAGGGCAGCAGCG
TGGCCACCCCTGGGCCACCTGTCGAGAGGGCTTCCTGCTACAACT
CCGCGTTGGGCTGCTGCTCTGATGGGAAGACGCCCTCGCTGGACG
CAGAGGGCTCCAACTGCCCCGCCACCAAGGTGTTCCAGGGCGTCC
TGGAGCTGGAGGGCGTCGAGGGCCAGGAGCTGTTCTACACGCCCG
AGATGGCTGACCCCAAGTCAGAACTGTTCGGGGAGACAGCCAGGA
GCATTGAGAGCACCCTGGACGACCTCTTCCGGAATTCAGACGTCA
AGAAGGATTTTCGGAGTGTCCGCTTGCGGGACCTGGGGCCCGGCA
AATCCGTCCGCGCCATTGTGGATGTGCACTTTGACCCCACCACAG
CCTTCAGGGCACCCGACGTGGCCCGGGCCCTGCTCCGGCAGATCC
AGGTGTCCAGGCGCCGGTCCTTGGGGGTGAGGCGGCCGCTGCAGG
AGCACGTGCGATTTATGGACTTTGACTGGTTTCCTGCGTTTATCA
CGGGGGCCACGTCAGGAGCCATTGCTGCGGGAGCCACGGCCAGAG
CCACCACTGCATCGCGCCTGCCGTCCTCTGCTGTGACCCCTCGGG
CCCCGCACCCCAGTCACACAAGCCAGCCCGTTGCCAAGACCACGG
CAGCCCCCACCACACGTCGGCCCCCCACCACTGCCCCCAGCCGTG
TGCCCGGACGTCGGCCCCCGGCCCCCCAGCAGCCTCCAAAGCCCT
GTGACTCACAGCCCTGCTTCCACGGGGGGACCTGCCAGGACTGGG
CATTGGGGGGGGGCTTCACCTGCAGCTGCCCGGCAGGCAGGGGAG
GCGCCGTCTGTGAGAAGGTGCTTGGCGCCCCTGTGCCGGCCTTCG
AGGGCCGCTCCTTCCTGGCCTTCCCCACTCTCCGCGCCTACCACA
CGCTGCGCCTGGCACTGGAATTCCGGGCGCTGGAGCCTCAGGGGC
TGCTGCTGTACAATGGCAACGCCCGGGGCAAGGACTTCCTGGCAT
TGGCGCTGCTAGATGGCCGCGTGCAGCTCAGGTTTGACACAGGTT
CGGGGCCGGCGGTGCTGACCAGTGCCGTGCCGGTAGAGCCGGGCC
AGTGGCACCGCCTGGAGCTGTCCCGGCACTGGCGCCGGGGCACCC
TCTCGGTGGATGGTGAGACCCCTGTTCTGGGCGAGAGTCCCAGTG
GCACCGACGGCCTCAACCTGGACACAGACCTCTTTGTGGGCGGCG
TACCCGAGGACCAGGCTGCCGTGGCGCTGGAGCGGACCTTCGTGG
GCGCCGGCCTGAGGGGGTGCATCCGTTTGCTGGACGTCAACAACC
AGCGCCTGGAGCTTGGCATTGGGCCGGGGGCTGCCACCCGAGGCT
CTGGCGTGGGCGAGTGCGGGGACCACCCCTGCCTGCCCAACCCCT
GCCATGGCGGGGCCCCATGCCAGAACCTGGAGGCTGGAAGGTTCC
ATTGCCAGTGCCCGCCCGGCCGCGTCGGACCAACCTGTGCCGATG
AGAAGAGCCCCTGCCAGCCCAACCCCTGCCATGGGGCGGCGCCCT
GCCGTGTGCTGCCCGAGGGTGGTGCTCAGTGCGAGTGCCCCCTGG
GGCGTGAGGGCACCTTCTGCCAGACAGCCTCGGGGCAGGACGGCT
CTGGGCCCTTCCTGGCTGACTTCAACGGCTTCTCCCACCTGGAGC
TGAGAGGCCTGCACACCTTTGCACGGGACCTGGGGGAGAAGATGG
CGCTGGAGGTCGTGTTCCTGGCACGAGGCCCCAGCGGCCTCCTGC
TCTACAACGGGCAGAAGACGGACGGCAAGGGGGACTTCGTGTCGC
TGGCACTGCGGGACCGCCGCCTGGAGTTCCGCTACGACCTGGGCA
AGGGGGCAGCGGTCATCAGGAGCAGGGAGCCAGTCACCCTGGGAG
CCTGGACCAGGGTCTCACTGGAGCGAAACGGCCGCAAGGGTGCCC
TGCGTGTGGGCGACGGCCCCCGTGTGTTGGGGGAGTCCCCGAAAT
CCCGCAAGGTTCCGCACACCGTCCTCAACCTGAAGGAGCCGCTCT
ACGTAGGGGGCGCTCCCGACTTCAGCAAGCTGGCCCGTGCTGCTG
CCGTGTCCTCTGGCTTCGACGGTGCCATCCAGCTGGTCTCCCTCG
GAGGCCGCCAGCTGCTGACCCCGGAGCACGTGCTGCGGCAGGTGG
ACGTCACGTCCTTTGCAGGTCACCCCTGCACCCGGGCCTCAGGCC
ACCCCTGCCTCAATGGGGCCTCCTGCGTCCCGAGGGAGGCTGCCT
ATGTGTGCCTGTGTCCCGGGGGATTCTCAGGACCGCACTGCGAGA
AGGGGCTGGTGGAGAAGTCAGCGGGGGACGTGGATACCTTGGCCT
TTGACGGGCGGACCTTTGTCGAGTACCTCAACGCTGTGACCGAGA
GCGAACTGGCCAATGAGATCCCCGTCCCCGAAACTCTGGATTCCG
GGGCCCTTCACAGCGAGAAGGCACTGCAGAGCAACCACTTTGAAC
TGAGCCTGCGCACTGAGGCCACGCAGGGGCTGGTGCTCTGGAGTG
GCAAGGCCACGGAGCGGGCAGACTATGTGGCACTGGCCATTGTGG
ACGGGCACCTGCAACTGAGCTACAACCTGGGCTCCCAGCCCGTGG
TGCTGCGTTCCACCGTGCCCGTCAACACCAACCGCTGGTTGCGGG
TCGTGGCACATAGGGAGCAGAGGGAAGGTTCCCTGCAGGTGGGCA
ATGAGGCCCCTGTGACCGGCTCCTCCCCGCTGGGCGCCACGCAGC
TGGACACTGATGGAGCCCTGTGGCTTGGGGGCCTGCCGGAGCTGC
CCGTGGGCCCAGCACTGCCCAAGGCCTACGGCACAGGCTTTGTGG
GCTGCTTGCGGGACGTGGTGGTGGGCCGGCACCCGCTGCACCTGC
TGGAGGACGCCGTCACCAAGCCAGAGCTGCGGCCCTGCCCCACCC
CATGAGCTGGCACCAGAGCCCCGCGCCCGCTGTAATTATTTTCTA
TTTTTGTAAACTTGTTGCTTTTTGATATGATTTTCTTGCCTGAGT
GTTGGCCGGAGGGACTGCTGGCCCGGCCTCCCTTCCGTCCAGGCA
GCCGTGCTGCAGACAGACCTAGTGCCGAGGGATGGACAGGCGAGG
TGGCAGCGTGGAGGGCTCGGCGTGGATGGCAGCCTCAGGACACAC
ACCCCTGCCTCAAGGTGCTGAGCCCCCGCCTTGCACTGCGCCTGC
CCCACGGTGTCCCCGCCGGGAAGCAGCCCCGGCTCCTGAATCACC
CTCGCTCCGTCAGGCGGGACTCGTGTCCCAGAGAGGAAGGGGCTG
CTGAGGTCTGATGGGGCCCTTCCTCCGGGTGACCCCACAGGGCCT
TTCCAAGCCCCCATTTGAGCTGCTCCTTCCTGTGTGTGCTCTGGG
CCCTGCCTCGGCCTCCTGCGCCAATACTGTGACTTCCAAACAATG
TTACTGCTGGGCACAGCTCTGCGTTGCTCCCGTGCTGCCTGCGCC
AGCCCCAGGCTGCTGAGGAGCAGAGGCCAGACCAGGGCCGATCTG
GGTGTCCTGACCCTCAGCTGGCCCTGCCCAGCCACCCTGGACGTG
ACCGTATCCCTCTGCCACACCCCAGGCCCTGCGAGGGGCTATCGA
GAGGAGCTCACTGTGGGATGGGGTTGACCTCTGCCGCCTGCCTGG
GTATCTGGGCCTGGCCATGGCTGTGTTCTTCATGTGTTGATTTTA
TTTGACCCCTGGAGTGGTGGGTCTCATCTTTCCCATCTCGCCTGA
GAGCGGCTGAGGGCTGCCTCACTGCAAATCCTCCCCACAGCGTCA
GTGAAAGTCGTCCTTGTCTCAGAATGACCAGGGGCCAGCCAGTGT
CTGACCAAGGTCAAGGGGCAGGTGCAGAGGTGGCAGGGATGGCTC
CGAAGCCAGAAATGCCTTAAACTGCAACGTCCCGTCCCTTCCCCA
CCCCCATCCCATCCCCACCCCCAGCCCCAGCCCAGTCCTCCTAGG
AGCAGGACCCGATGAAGCGGGCGGCGGTGGGGCTGGGTGCCGTGT
TACTAACTCTAGTATGTTTCTGTGTCAATCGCTGTGAAATAAAGT
CTGAAAACTTTAAAA

### PVALB

SEQ ID NO: 217:
PVALB-NRG1 fusion sequence
TAAAAGGCTTCTCCCCAGATGCCAGAGACCTGTCTGCTAAAGAAA
CCAAGATGCTGATGGCTGCTGGAGACAAAGATGGGGACGGCAAAA
TTGGGGTTGACGCTACATCTACATCCACCACTGGGACAAGCCATC
TTGTAAAATGTGCGGAGAAGGAGAAAACTTTCTGTGTGAATGGAG
GGGAGTGCTTCATGGTGAAAGACCTTTCAAACCCCTCGAGATACT
TG

SEQ ID NO: 218=Exon 1 PVALB
SEQ ID NO: 219=Exon 2 PVALB
SEQ ID NO: 220=Exon 3 PVALB
SEQ ID NO: 221=Exon 4 PVALB
SEQ ID NO: 222=Exon 5 PVALB
SEQ ID NO: 223=Exon 1-5 PVALB
SEQ ID NO: 224=Exon 1-4 PVALB

PVALB sequence information, providing nucleotide sequence of exons 1-5 below as indicated by alternating underlining, starting with exon 1 underlined, exon 2 not underlined, exon 3 underlined, exon 4 not underlined, and so forth.

SEQ ID NO: 223
ACTTCCCGACAGGACTTCCCACCAGCCCAGCCTTTCAGTGCAGGC
TCCAGCCCTCCACCCCCACCCGAGTTGCAGGATGTCGATGACAGA
CTTGCTGAACGCTGAGGACATCAAGAAGGCGGTGGGAGCCTTTAG
CGCTACCGACTCCTTCGACCACAAAAAGTTCTTCCAAATGGTCGG
CCTGAAGAAAAAGAGTGCGGATGATGTGAAGAAGGTGTTTCACAT
GCTGGACAAGGACAAAAGTGGCTTCATCGAGGAGGATGAGCTGGG
ATTCATCCTAAAAGGCTTCTCCCCAGATGCCAGAGACCTGTCTGC
TAAAGAAACCAAGATGCTGATGGCTGCTGGAGACAAAGATGGGGA
CGGCAAAATTGGGGTTGACGAATTCTCCACTCTGGTGGCTGAAAG
CTAAGAAGCACTGACTGCCCCTGGTCTTCCACCTCTCTGCCCTGA
ACACCCAATCTCGGCCCCTCTCGCCACCCTCCTGCATTTCTGTTC
AGTTCGTTTATGTTATTTTTTACTCCCCCATCCCCTGTGGCCCTC
TAATGACACCATTCTTCTGGAAAATGCTGGAGAAGCAATAAAGGT
TGTACCAGTCA

### SLC3A2

SLC3A2-NRG1 fusion sequence
SEQ ID NO: 225
AGTTGGGGTCTCACTGTGTTGCCCAGACTGGTCTCGAACTCTTGG
CCTCAGGTGATCCTCTTCCCTCAGCTTCCCAGAATGCCGAGATGA
TAGCTACATCTACATCCACCACTGGGACAAG

SLC3A2 transcript version 3 sequence information, providing nucleotide sequence of exons 1-12 below as indicated by alternating underlining, starting with exon 1 underlined, exon 2 not underlined, exon 3 underlined, exon 4 not underlined, and so forth.
SEQ ID NO: 226=Exon 1 SLC3A2 transcript version 3
SEQ ID NO: 227=Exon 2 SLC3A2 transcript version 3
SEQ ID NO: 228=Exon 3 SLC3A2 transcript version 3
SEQ ID NO: 229=Exon 4 SLC3A2 transcript version 3
SEQ ID NO: 230=Exon 5 SLC3A2 transcript version 3
SEQ ID NO: 231=Exon 6 SLC3A2 transcript version 3
SEQ ID NO:232=Exon 7 SLC3A2 transcript version 3
SEQ ID NO:233=Exon 8 SLC3A2 transcript version 3
SEQ ID NO:234=Exon 9 SLC3A2 transcript version 3
SEQ ID NO:235=Exon 10 SLC3A2 transcript version 3
SEQ ID NO:236=Exon 11 SLC3A2 transcript version 3
SEQ ID NO: 237=Exon 12 SLC3A2 transcript version 3
SEQ ID NO: 238=Exons 1-12 SLC3A2 transcript version 3
SEQ ID NO:239=Exon 1-2 SLC3A2 transcript version 3

SEQ ID NO: 238
GCATTGCGGCTTGGTTTTCTCACCCAGTGCATGTGGCAGGAGCGG
TGAGATCACTGCCTCACGGCGATCCTGGACTGACGGTCACGACTG
CCTACCCTCTAACCCTGTTCTGAGCTGCCCCTTGCCCACACACCC
CAAACCTGTGTGCAGGATCCGCCTCCATGGAGCTACAGCCTCCTG
AAGCCTCGATCGCCGTCGTGTCGATTCCGCGCCAGTTGCCTGGCT
CACATTCGGAGGCTGGTGTCCAGGGTCTCAGCGCGGGGGACGACT
CAGAGTTGGGGTCTCACTGTGTTGCCCAGACTGGTCTCGAACTCT
TGGCCTCAGGTGATCCTCTTCCCTCAGCTTCCCAGAATGCCGAGA
TGATAGAGACGGGGTCTGACTGTGTTACCCAGGCTGGTCTTCAAC
TCTTGGCCTCAAGTGATCCTCCTGCCTTAGCTTCCAAGAATGCTG
AGGTTACAGGCACCATGAGCCAGGACACCGAGGTGGATATGAAGG
AGGTGGAGCTGAATGAGTTAGAGCCCGAGAAGCAGCCGATGAACG
CGGCGTCTGGGGCGGCCATGTCCCTGGCGGGAGCCGAGAAGAATG
GTCTGGTGAAGATCAAGGTGGCGGAAGACGAGGCGGAGGCGGCAG
CCGCGGCTAAGTTCACGGGCCTGTCCAAGGAGGAGCTGCTGAAGG
TGGCAGGCAGCCCCGGCTGGGTACGCACCCGCTGGGCACTGCTGC
TGCTCTTCTGGCTCGGCTGGCTCGGCATGCTTGCTGGTGCCGTGG
TCATAATCGTGCGAGCGCCGCGTTGTCGCGAGCTACCGGCGCAGA
AGTGGTGGCACACGGGCGCCCTCTACCGCATCGGCGACCTTCAGG
CCTTCCAGGGCCACGGCGCGGGCAACCTGGCGGGTCTGAAGGGGC
GTCTCGATTACCTGAGCTCTCTGAAGGTGAAGGGCCTTGTGCTGG
GTCCAATTCACAAGAACCAGAAGGATGATGTCGCTCAGACTGACT
TGCTGCAGATCGACCCCAATTTTGGCTCCAAGGAAGATTTTGACA
GTCTCTTGCAATCGGCTAAAAAAAAGAGCATCCGTGTCATTCTGG
ACCTTACTCCCAACTACCGGGGTGAGAACTCGTGGTTCTCCACTC
AGGTTGACACTGTGGCCACCAAGGTGAAGGATGCTCTGGAGTTTT
GGCTGCAAGCTGGCGTGGATGGGTTCCAGGTTCGGGACATAGAGA
ATCTGAAGGATGCATCCTCATTCTTGGCTGAGTGGCAAAATATCA
CCAAGGGCTTCAGTGAAGACAGGCTCTTGATTGCGGGGACTAACT
CCTCCGACCTTCAGCAGATCCTGAGCCTACTCGAATCCAACAAAG
ACTTGCTGTTGACTAGCTCATACCTGTCTGATTCTGGTTCTACTG
GGGAGCATACAAAATCCCTAGTCACACAGTATTTGAATGCCACTG
GCAATCGCTGGTGCAGCTGGAGTTTGTCTCAGGCAAGGCTCCTGA
CTTCCTTCTTGCCGGCTCAACTTCTCCGACTCTACCAGCTGATGC
TCTTCACCCTGCCAGGGACCCCTGTTTTCAGCTACGGGGATGAGA
TTGGCCTGGATGCAGCTGCCCTTCCTGGACAGCCTATGGAGGCTC
CAGTCATGCTGTGGGATGAGTCCAGCTTCCCTGACATCCCAGGGG
CTGTAAGTGCCAACATGACTGTGAAGGGCCAGAGTGAAGACCCTG
GCTCCCTCCTTTCCTTGTTCCGGCGGCTGAGTGACCAGCGGAGTA
AGGAGCGCTCCCTACTGCATGGGGACTTCCACGCGTTCTCCGCTG
GGCCTGGACTCTTCTCCTATATCCGCCACTGGGACCAGAATGAGC
GTTTTCTGGTAGTGCTTAACTTTGGGGATGTGGGCCTCTCGGCTG
GACTGCAGGCCTCCGACCTGCCTGCCAGCGCCAGCCTGCCAGCCA
AGGCTGACCTCCTGCTCAGCACCCAGCCAGGCCGTGAGGAGGGCT
CCCCTCTTGAGCTGGAACGCCTGAAACTGGAGCCTCACGAAGGGC
TGCTGCTCCGCTTCCCCTACGCGGCCTGACTTCAGCCTGACATGG
ACCCACTACCCTTCTCCTTTCCTTCCCAGGCCCTTTGGCTTCTGA
TTTTTCTCTTTTTTAAAAACAAACAAACAAACTGTTGCAGATTAT
GAGTGAACCCCCAAATAGGGTGTTTTCTGCCTTCAAATAAAAGTC
ACCCCTGCATGGTGAA

### APP

APP-NRG1 fusion sequence
SEQ ID NO: 240
TTGAGCCTGTTGATGCCCGCCCTGCTGCCGACCGAGGACTGACCAC
TCGACCAGCTACATCTACATCCACCACTGGGACAAGCCATCTTGT
AAAATGTGCGGAGAAGGAGAAAACTTTCTGTGTGAATGGAGGGGA
GTGCT

SEQ ID NO:241=Exon 1 APP
SEQ ID NO:242=Exon 2 APP
SEQ ID NO:243=Exon 3 APP
SEQ ID NO: 244=Exon 4 APP
SEQ ID NO: 245=Exon 5 APP
SEQ ID NO: 246=Exon 6 APP
SEQ ID NO: 247=Exon 7 APP
SEQ ID NO: 248=Exon 8 APP
SEQ ID NO: 249=Exon 9 APP
SEQ ID NO: 250=Exon 10 APP
SEQ ID NO: 251=Exon 11 APP
SEQ ID NO: 252=Exon 12 APP
SEQ ID NO:253=Exon 13 APP
SEQ ID NO:254=Exon 14 APP
SEQ ID NO: 255=Exon 15 APP
SEQ ID NO: 256=Exon 16 APP
SEQ ID NO: 257=Exon 17 APP
SEQ ID NO:258=Exons 1-17 APP
SEQ ID NO:259=Exon 1-14 APP

APP sequence information, providing nucleotide sequence of exons 1-17 below as indicated by alternating underlining, starting with exon 1 underlined, exon 2 not underlined, exon 3 underlined, exon 4 not underlined, and so forth.

SEQ ID NO: 258
GTCAGTTTCCTCGGCAGCGGTAGGCGAGAGCACGCGGAGGAGCGT
GCGCGGGGGCCCCGGGAGACGGCGGCGGTGGCGGCGCGGGCAGAG
CAAGGACGCGGCGGATCCCACTCGCACAGCAGCGCACTCGGTGCC
CCGCGCAGGGTCGCGATGCTGCCCGGTTTGGCACTGCTCCTGCTG
GCCGCCTGGACGGCTCGGGCGCTGGAGGTCTACCCTGAACTGCAG
ATCACCAATGTGGTAGAAGCCAACCAACCAGTGACCATCCAGAAC
TGGTGCAAGCGGGGCCGCAAGCAGTGCAAGACCCATCCCCACTTT
GTGATTCCCTACCGCTGCTTAGTTGGTGAGTTTGTAAGTGATGCC
CTTCTCGTTCCTGACAAGTGCAAATTCTTACACCAGGAGAGGATG
GATGTTTGCGAAACTCATCTTCACTGGCACACCGTCGCCAAAGAG
ACATGCAGTGAGAAGAGTACCAACTTGCATGACTACGGCATGTTG
CTGCCCTGCGGAATTGACAAGTTCCGAGGGGTAGAGTTTGTGTGT
TGCCCACTGGCTGAAGAAAGTGACAATGTGGATTCTGCTGATGCG
GAGGAGGATGACTCGGATGTCTGGTGGGGCGGAGCAGACACAGAC
TATGCAGATGGGAGTGAAGACAAAGTAGTAGAAGTAGCAGAGGAG
GAAGAAGTGGCTGAGGTGGAAGAAGAAGAAGCCGATGATGACGAG
GACGATGAGGATGGTGATGAGGTAGAGGAAGAGGCTGAGGAACCC
TACGAAGAAGCCACAGAGAGAACCACCAGCATTGCCACCACCACC
ACCACCACCACAGAGTCTGTGGAAGAGGTGGTTCGAGAGGTGTGC
TCTGAACAAGCCGAGACGGGGCCGTGCCGAGCAATGATCTCCCGC
TGGTACTTTGATGTGACTGAAGGGAAGTGTGCCCCATTCTTTTAC
GGCGGATGTGGCGGCAACCGGAACAACTTTGACACAGAAGAGTAC
TGCATGGCCGTGTGTGGCAGCGCCATGTCCCAAAGTTTACTCAAG
ACTACCCAGGAACCTCTTGCCCGAGATCCTGTTAAACTTCCTACA
ACAGCAGCCAGTACCCCTGATGCCGTTGACAAGTATCTCGAGACA
CCTGGGGATGAGAATGAACATGCCCATTTCCAGAAAGCCAAAGAG
AGGCTTGAGGCCAAGCACCGAGAGAGAATGTCCCAGGTCATGAGA
GAATGGGAAGAGGCAGAACGTCAAGCAAAGAACTTGCCTAAAGCT
GATAAGAAGGCAGTTATCCAGCATTTCCAGGAGAAAGTGGAATCT
TTGGAACAGGAAGCAGCCAACGAGAGACAGCAGCTGGTGGAGACA
CACATGGCCAGAGTGGAAGCCATGCTCAATGACCGCCGCCGCCTG
GCCCTGGAGAACTACATCACCGCTCTGCAGGCTGTTCCTCCTCGG
CCTCGTCACGTGTTCAATATGCTAAAGAAGTATGTCCGCGCAGAA
CAGAAGGACAGACAGCACACCCTAAAGCATTTCGAGCATGTGCGC
ATGGTGGATCCCAAGAAAGCCGCTCAGATCCGGTCCCAGGTTATG
ACACACCTCCGTGTGATTTATGAGCGCATGAATCAGTCTCTCTCC
CTGCTCTACAACGTGCCTGCAGTGGCCGAGGAGATTCAGGATGAA
GTTGATGAGCTGCTTCAGAAAGAGCAAAACTATTCAGATGACGTC
TTGGCCAACATGATTAGTGAACCAAGGATCAGTTACGGAAACGAT
GCTCTCATGCCATCTTTGACCGAAACGAAAACCACCGTGGAGCTC
CTTCCCGTGAATGGAGAGTTCAGCCTGGACGATCTCCAGCCGTGG
CATTCTTTTGGGGCTGACTCTGTGCCAGCCAACACAGAAAACGAA
GTTGAGCCTGTTGATGCCCGCCCTGCTGCCGACCGAGGACTGACC
ACTCGACCAGGTTCTGGGTTGACAAATATCAAGACGGAGGAGATC
TCTGAAGTGAAGATGGATGCAGAATTCCGACATGACTCAGGATAT
GAAGTTCATCATCAAAAATTGGTGTTCTTTGCAGAAGATGTGGGT
TCAAACAAAGGTGCAATCATTGGACTCATGGTGGGCGGTGTTGTC
ATAGCGACAGTGATCGTCATCACCTTGGTGATGCTGAAGAAGAAA
CAGTACACATCCATTCATCATGGTGTGGTGGAGGTTGACGCCGCT
GTCACCCCAGAGGAGCGCCACCTGTCCAAGATGCAGCAGAACGGC
TACGAAAATCCAACCTACAAGTTCTTTGAGCAGATGCAGAACTAG
ACCCCCGCCACAGCAGCCTCTGAAGTTGGACAGCAAAACCATTGC
TTCACTACCCATCGGTGTCCATTTATAGAATAATGTGGGAAGAAA
CAAACCCGTTTTATGATTTACTCATTATCGCCTTTTGACAGCTGT
GCTGTAACACAAGTAGATGCCTGAACTTGAATTAATCCACACATC
AGTAATGTATTCTATCTCTCTTTACATTTTGGTCTCTATACTACA
TTATTAATGGGTTTTGTGTACTGTAAAGAATTTAGCTGTATCAAA
CTAGTGCATGAATAGATTCTCTCCTGATTATTTATCACATAGCCC
CTTAGCCAGTTGTATATTATTCTTGTGGTTTGTGACCCAATTAAG
TCCTACTTTACATATGCTTTAAGAATCGATGGGGGATGCTTCATG
TGAACGTGGGAGTTCAGCTGCTTCTCTTGCCTAAGTATTCCTTTC
CTGATCACTATGCATTTTAAAGTTAAACATTTTTAAGTATTTCAG
ATGCTTTAGAGAGATTTTTTTTCCATGACTGCATTTTACTGTACA
GATTGCTGCTTCTGCTATATTTGTGATATAGGAATTAAGAGGATA
CACACGTTTGTTTCTTCGTGCCTGTTTTATGTGCACACATTAGGC
ATTGAGACTTCAAGCTTTTCTTTTTTTGTCCACGTATCTTTGGGT
CTTTGATAAAGAAAAGAATCCCTGTTCATTGTAAGCACTTTTACG
GGGCGGGTGGGGAGGGGTGCTCTGCTGGTCTTCAATTACCAAGAA
TTCTCCAAAACAATTTTCTGCAGGATGATTGTACAGAATCATTGC
TTATGACATGATCGCTTTCTACACTGTATTACATAAATAAATTAA
ATAAAATAACCCCGGGCAAGACTTTTCTTTGAAGGATGACTACAG
ACATTAAATAATCGAAGTAATTTTGGGTGGGGAGAAGAGGCAGAT
TCAATTTTCTTTAACCAGTCTGAAGTTTCATTTATGATACAAAAG
AAGATGAAAATGGAAGTGGCAATATAAGGGGATGAGGAAGGCATG
CCTGGACAAACCCTTCTTTTAAGATGTGTCTTCAATTTGTATAAA
ATGGTGTTTTCATGTAAATAAATACATTCTTGGAGGAGCA

### WRN

WRN-NRG1 fusion sequence
SEQ ID NO: 260
AAGCTGGCTGCCCCCTTGATTTGGAGCGAGCAGGCCTGACTCCAG
AGGTTCAGAAGATTATTGCTGATGTTATCCGAAACCCTCCCGTCA
ACTCAGCTACATCTACATCCACCACTGGGACAAGCCATCTTGTAA
AATGTGCGGAGAAGGAGAAAACTTTCTGTGTGAATGGAGGGGAGT
GC

SEQ ID NO: 261=WRN Exon 1
SEQ ID NO: 262=WRN Exon 2
SEQ ID NO: 263=WRN Exon 3
SEQ ID NO: 264=WRN Exon 4
SEQ ID NO: 265=WRN Exon 5
SEQ ID NO: 266=WRN Exon 6
SEQ ID NO: 267=WRN Exon 7
SEQ ID NO: 268=WRN Exon 8
SEQ ID NO: 269=WRN Exon 9
SEQ ID NO: 270=WRN Exon 10
SEQ ID NO: 271=WRN Exon 11
SEQ ID NO: 272=WRN Exon 12
SEQ ID NO: 273=WRN Exon 13
SEQ ID NO: 274=WRN Exon 14
SEQ ID NO: 275=WRN Exon 15
SEQ ID NO: 276=WRN Exon 16
SEQ ID NO: 277=WRN Exon 17
SEQ ID NO: 278=WRN Exon 18
SEQ ID NO: 279=WRN Exon 19
SEQ ID NO: 280=WRN Exon 20
SEQ ID NO: 281=WRN Exon 21
SEQ ID NO: 282=WRN Exon 22
SEQ ID NO: 283=WRN Exon 23
SEQ ID NO: 284=WRN Exon 24
SEQ ID NO: 285=WRN Exon 25
SEQ ID NO: 286=WRN Exon 26
SEQ ID NO: 287=WRN Exon 27
SEQ ID NO: 288=WRN Exon 28
SEQ ID NO: 289=WRN Exon 29
SEQ ID NO: 290=WRN Exon 30
SEQ ID NO: 291=WRN Exon 31
SEQ ID NO: 292=WRN Exon 32
SEQ ID NO: 293=WRN Exon 33
SEQ ID NO: 294=WRN Exon 34
SEQ ID NO: 295=WRN Exon 35
SEQ ID NO: 296=WRN Exons 1-35 WRN
SEQ ID NO: 297=WRN Exons 1-33 WRN

WRN sequence information, providing nucleotide sequence of exons 1-35 below as indicated by alternating underlining, starting with exon 1 underlined, exon 2 not underlined, exon 3 underlined, exon 4 not underlined, and so forth.

SEQ ID NO: 296
GTGTACTGTGTGCGCCGGGGAGGCGCCGGCTTGTACTCGGCAGCG
CGGGAATAAAGTTTGCTGATTTGGTGTCTAGCCTGGATGCCTGGG
TTGCAGGCCCTGCTTGTGGTGGCGCTCCACAGTCATCCGGCTGAA
GAAGACCTGTTGGACTGGATCTTCTCGGGTTTTCTTTCAGATATT
GTTTTGTATTTACCCATGAAGACATTGTTTTTTGGACTCTGCAAA
TAGGACATTTCAAAGATGAGTGAAAAAAAATTGGAAACAACTGCA
CAGCAGCGGAAATGTCCTGAATGGATGAATGTGCAGAATAAAAGA
TGTGCTGTAGAAGAAAGAAAGGCATGTGTTCGGAAGAGTGTTTTT
GAAGATGACCTCCCCTTCTTAGAATTCACTGGATCCATTGTGTAT
AGTTACGATGCTAGTGATTGCTCTTTCCTGTCAGAAGATATTAGC
ATGAGTCTATCAGATGGGGATGTGGTGGGATTTGACATGGAGTGG
CCACCATTATACAATAGAGGGAAACTTGGCAAAGTTGCACTAATT
CAGTTGTGTGTTTCTGAGAGCAAATGTTACTTGTTCCACGTTTCT
TCCATGTCAGTTTTTCCCCAGGGATTAAAAATGTTGCTTGAAAAT
AAAGCAGTTAAAAAGGCAGGTGTAGGAATTGAAGGAGATCAGTGG
AAACTTCTACGTGACTTTGATATCAAATTGAAGAATTTTGTGGAG
TTGACAGATGTTGCCAATAAAAAGCTGAAATGCACAGAGACCTGG
AGCCTTAACAGTCTGGTTAAACACCTCTTAGGTAAACAGCTCCTG
AAAGACAAGTCTATCCGCTGTAGCAATTGGAGTAAATTTCCTCTC
ACTGAGGACCAGAAACTGTATGCAGCCACTGATGCTTATGCTGGT
TTTATTATTTACCGAAATTTAGAGATTTTGGATGATACTGTGCAA
AGGTTTGCTATAAATAAAGAGGAAGAAATCCTACTTAGCGACATG
AACAAACAGTTGACTTCAATCTCTGAGGAAGTGATGGATCTGGCT
AAGCATCTTCCTCATGCTTTCAGTAAATTGGAAAACCCACGGAGG
GTTTCTATCTTACTAAAGGATATTTCAGAAAATCTATATTCACTG
AGGAGGATGATAATTGGGTCTACTAACATTGAGACTGAACTGAGG
CCCAGCAATAATTTAAACTTATTATCCTTTGAAGATTCAACTACT
GGGGGAGTACAACAGAAACAAATTAGAGAACATGAAGTTTTAATT
CACGTTGAAGATGAAACATGGGACCCAACACTTGATCATTTAGCT
AAACATGATGGAGAAGATGTACTTGGAAATAAAGTGGAACGAAAA
GAAGATGGATTTGAAGATGGAGTAGAAGACAACAAATTGAAAGAG
AATATGGAAAGAGCTTGTTTGATGTCGTTAGATATTACAGAACAT
GAACTCCAAATTTTGGAACAGCAGTCTCAGGAAGAATATCTTAGT
GATATTGCTTATAAATCTACTGAGCATTTATCTCCCAATGATAAT
GAAAACGATACGTCCTATGTAATTGAGAGTGATGAAGATTTAGAA
ATGGAGATGCTTAAGCATTTATCTCCCAATGATAATGAAAACGAT
ACGTCCTATGTAATTGAGAGTGATGAAGATTTAGAAATGGAGATG
CTTAAGTCTTTAGAAAACCTCAATAGTGGCACGGTAGAACCAACT
CATTCTAAATGCTTAAAAATGGAAAGAAATCTGGGTCTTCCTACT
AAAGAAGAAGAAGAAGATGATGAAAATGAAGCTAATGAAGGGGAA
GAAGATGATGATAAGGACTTTTTGTGGCCAGCACCCAATGAAGAG
CAAGTTACTTGCCTCAAGATGTACTTTGGCCATTCCAGTTTTAAA
CCAGTTCAGTGGAAAGTGATTCATTCAGTATTAGAAGAAAGAAGA
GATAATGTTGCTGTCATGGCAACTGGATATGGAAAGAGTTTGTGC
TTCCAGTATCCACCTGTTTATGTAGGCAAGATTGGCCTTGTTATC
TCTCCCCTTATTTCTCTGATGGAAGACCAAGTGCTACAGCTTAAA
ATGTCCAACATCCCAGCTTGCTTCCTTGGATCAGCACAGTCAGAA
AATGTTCTAACAGATATTAAATTAGGTAAATACCGGATTGTATAC
GTAACTCCAGAATACTGTTCAGGTAACATGGGCCTGCTCCAGCAA
CTTGAGGCTGATATTGGTATCACGCTCATTGCTGTGGATGAGGCT
CACTGTATTTCTGAGTGGGGGCATGATTTTAGGGATTCATTCAGG
AAGTTGGGCTCCCTAAAGACAGCACTGCCAATGGTTCCAATCGTT
GCACTTACTGCTACTGCAAGTTCTTCAATCCGGGAAGACATTGTA
CGTTGCTTAAATCTGAGAAATCCTCAGATCACCTGTACTGGTTTT
GATCGACCAAACCTGTATTTAGAAGTTAGGCGAAAAACAGGGAAT
ATCCTTCAGGATCTGCAGCCATTTCTTGTCAAAACAAGTTCCCAC
TGGGAATTTGAAGGTCCAACAATCATCTACTGTCCTTCTAGAAAA
ATGACACAACAAGTTACAGGTGAACTTAGGAAACTGAATCTATCC
TGTGGAACATACCATGCGGGCATGAGTTTTAGCACAAGGAAAGAC
ATTCATCATAGGTTTGTAAGAGATGAAATTCAGTGTGTCATAGCT
ACCATAGCTTTTGGAATGGGCATTAATAAAGCTGACATTCGCCAA
GTCATTCATTACGGTGCTCCTAAGGACATGGAATCATATTATCAG
GAGATTGGTAGAGCTGGTCGTGATGGACTTCAAAGTTCTTGTCAC
GTCCTCTGGGCTCCTGCAGACATTAACTTAAATAGGCACCTTCTT
ACTGAGATACGTAATGAGAAGTTTCGATTATACAAATTAAAGATG
ATGGCAAAGATGGAAAAATATCTTCATTCTAGCAGATGTAGGAGA
CAAATCATCTTGTCTCATTTTGAGGACAAACAAGTACAAAAAGCC
TCCTTGGGAATTATGGGAACTGAAAAATGCTGTGATAATTGCAGG
TCCAGATTGGATCATTGCTATTCCATGGATGACTCAGAGGATACA
TCCTGGGACTTTGGTCCACAAGCATTTAAGCTTTTGTCTGCTGTG
GACATCTTAGGCGAAAAATTTGGAATTGGGCTTCCAATTTTATTT
CTCCGAGGATCTAATTCTCAGCGTCTTGCCGATCAATATCGCAGG
CACAGTTTATTTGGCACTGGCAAGGATCAAACAGAGAGTTGGTGG
AAGGCTTTTTCCCGTCAGCTGATCACTGAGGGATTCTTGGTAGAA
GTTTCTCGGTATAACAAATTTATGAAGATTTGCGCCCTTACGAAA
AAGGGTAGAAATTGGCTTCATAAAGCTAATACAGAATCTCAGAGC
CTCATCCTTCAAGCTAATGAAGAATTGTGTCCAAAGAAGTTGCTT
CTGCCTAGTTCGAAAACTGTATCTTCGGGCACCAAAGAGCATTGT
TATAATCAAGTACCAGTTGAATTAAGTACAGAGAAGAAGTCTAAC
TTGGAGAAGTTATATTCTTATAAACCATGTGATAAGATTTCTTCT
GGGAGTAACATTTCTAAAAAAAGTATCATGGTACAGTCACCAGAA
AAAGCTTACAGTTCCTCACAGCCTGTTATTTCGGCACAAGAGCAG
GAGACTCAGATTGTGTTATATGGCAAATTGGTAGAAGCTAGGCAG
AAACATGCCAATAAAATGGATGTTCCCCCAGCTATTCTGGCAACA
AACAAGATACTGGTGGATATGGCCAAAATGAGACCAACTACGGTT
GAAAACGTAAAAAGGATTGATGGTGTTTCTGAAGGCAAAGCTGCC
ATGTTGGCCCCTCTGTTGGAAGTCATCAAACATTTCTGCCAAACA
AATAGTGTTCAGACAGACCTCTTTTCAAGTACAAAACCTCAAGAA
GAACAGAAGACGAGTCTGGTAGCAAAAAATAAAATATGCACACTT
TCACAGTCTATGGCCATCACATACTCTTTATTCCAAGAAAAGAAG
ATGCCTTTGAAGAGCATAGCTGAGAGCAGGATTCTGCCTCTCATG
ACAATTGGCATGCACTTATCCCAAGCGGTGAAAGCTGGCTGCCCC
CTTGATTTGGAGCGAGCAGGCCTGACTCCAGAGGTTCAGAAGATT
ATTGCTGATGTTATCCGAAACCCTCCCGTCAACTCAGATATGAGT
AAAATTAGCCTAATCAGAATGTTAGTTCCTGAAAACATTGACACG
TACCTTATCCACATGGCAATTGAGATCCTTAAACATGGTCCTGAC
AGCGGACTTCAACCTTCATGTGATGTCAACAAAAGGAGATGTTTT
CCCGGTTCTGAAGAGATCTGTTCAAGTTCTAAGAGAAGCAAGGAA
GAAGTAGGCATCAATACTGAGACTTCATCTGCAGAGAGAAAGAGA
CGATTACCTGTGTGGTTTGCCAAAGGAAGTGATACCAGCAAGAAA
TTAATGGACAAAACGAAAAGGGGAGGTCTTTTTAGTTAAGCTGGC
AATTACCAGAACAATTATGTTTCTTGCTGTATTATAAGAGGATAG
CTATATTTTATTTCTGAAGAGTAAGGAGTAGTATTTTGGCTTAAA
AATCATTCTAATTACAAAGTTCACTGTTTATTGAAGAACTGGCAT
CTTAAATCAGCCTTCCGCAATTCATGTAGTTTCTGGGTCTTCTGG
GAGCCTACGTGAGTACATCACCTAACAGAATATTAAATTAGACTT
CCTGTAAGATTGCTTTAAGAAACTGTTACTGTCCTGTTTTCTAAT
CTCTTTATTAAAACAGTGTATTTGGAAAATGTTATGTGCTCTGAT
TTGATATAGATAACAGATTAGTAGTTACATGGTAATTATGTGATA
TAAAATATTCATATATTATCAAAATTCTGTTTTGTAAATGTAAGA
AAGCATAGTTATTTTACAAATTGTTTTTACTGTCTTTTGAAGAAG
TTCTTAAATACGTTGTTAAATGGTATTAGTTGACCAGGGCAGTGA
AAATGAAACCGCATTTTGGGTGCCATTAAATAGGGAAAAAACATG
TAAAAAATGTAAAATGGAGACCAATTGCACTAGGCAAGTGTATAT
TTTGTATTTTATATACAATTTCTATTATTTTTCAAGTAATAAAAC
AATGTTTTTCATACTGAATATTATATATATATTTTTTAGCTTTCA
TTTACTTAATTATTTTAAGTACCTTTATTTTTCCAGGATGTCAGA
ATTTGATTCTAATCTCTCTTATGTAGCACATGTGACTTAATTTAA
AACCTATACTGTGACACAGAGTTGGGTAAACGATGATTATTTAAC
TTTAAGCAGTTCACCATCCATTTCAAAGCCTTTGATTGGCTTTTT
TGTAAATAAAAATAACTTGTTAAGAAACAAATATATCTGTCATAG
AAGAACTAGAAAATCCAGGGAAGTGAGAAAAATGAAAATAAAAAT
CATTCATAGTTTTACTAGTAGCTAATCACAGTCAACCTCTTTTGT
GTATCCCACCAGACTTTTTTATATTCATTTGTTTTTAGTTAAAAT
ATAAAAGTCTCGTATATTCCCATTTTTCTGCATTGCATTACCAGA
AGGTAGTGGCGCCTATTAAATATGTGATATGTTGTTGTCCAGCCA
TGGCTTCTGCATTTGCATGCTTTTGTGTGTGCATCTGCAATACCC
TGTGAATATCCTGTGTGATGGAGTGGCAAGTACGCACAGACACGT
CTGCTGCATGCCTAGGTACGAGGCTGTCTCCAGGAGAAGCACTTG
TTTGATTATTTGAGTTGCCAATTGAATTTGCTGCTTTTTTTCATG
GCTTGCCATTTTCACTGAAAAGAATGACTAATGAAAAACGATGAT
TGGTTATTAGATTTGGATGTTTGGCAGACATTTTCTCAAAATTGA
ACTAAGTTGGCCTCTTCACGGAAAACAACTGGTATTTGTTGTGCC
AATGATAAAATTGGAGATTTCTAGCAAAATGTATAATTTTGGAAA
AGTTGTGTTCCTCCACTGGAAGCTTGACAGCTTTCCTTAACATAA
AGACTTCTCTTTCTCTTCGCTTTCACTACTACTACTACTAATTCT
TCTTCTGATTCTTCTTCTTCTCCTTCTTCCTTCTTCCTTCCTTCC
TCCTCCTTCCTTCCTTCCTTCCTCCCTCCCTCCCTCCCTCCCTCC
CTCCCTCCTTTCTTTTTCTTTCTCTTTCTTTCTTTCTTTCTCTCT
CTCTCTCTCTTTCTTTCTTTTTCTTTCTCTTTTTCTTTCTTTCAA
GCAGTCCTCCCGCCTCAGTCCCCCAAAATAGTGGGATTACAGGTG
TGAGCCACCATGCACAGCCTTACATAAAGCCTTTTCTAATGAGAT
GGATAGTAATTAACAAATGTGAGTTTTTGATATTATATAAAGATT
TTTTCTGTGTTTCGAAGATCCGTATAACTCAGTGAATCAGTATGT
TCTGGATGACTAATATGTGATGTTAAGAAATCATGACTGAGGCCG
GGCGCGGTGGCTCACGCCTGTAATCCCAGCACTTTGGGAGGCCGA
GGCGGGCGGATCACGAGATCAGGAGATCGAGACCACCCTGGCCAA
CATGGTGAAACCCCGTCTCTACTAAAAATACAAAAATTAGCTGGG
TGTGTTGGTGCGTGCCTATAATCCCAGCTACTCGGGAGGCTGAGG
CAGGAGAATCGCTTGAACTCAGGAGGCGGAGATTGCAGTGAGCTG
AGACTGCGCCACTGCACCCCAGCCTGGCGACAGAGCAAGACTCCG
TCTCAAAAATAAAAAAAGAAATCATGACTGGGTAAAAGATCTGTT
CAGAGTACAAGATGGACCAATGGATTTGATATATTTGAATATAAC
AGAGTATGAAAAAGTTTATTGATATAGTTTCAGATTACACACTGC
AACTAATCTTTAAGAAACTATTACTTGTCCACTTTTTGGTAAAAT
TTCAGAGAACAATGTCCACCATTATCTGAACAGGCTATTAAAATA
CTCTTCTCTTTTCCAACTACGTGCCTGTGCAAAGTCAGATTTTTT
TCATATACTTCAGCCAAAACAGCATATCAAAATGGATTGAATGCA
GAAGTAGATCTGAGAATACAGCCACTTTTGTTAAGCCAGACAATG
AGATTTGCAAAATGTAAACAATGCTGCTGTTCTCAGTTTTTAAAA
ATATGTTTTTTAAAAGTATTTATGTTAATGTGTACTTGGTTTACT
ACTGCTATTTTTAAATAAAACAAGAAACATTTTTAAATGTCTGTT
TTAATTTCTAAAGTGGTAGTGATAGATATAACCCATATTAATAAA
AGCTCTTTGGGGTCCTCAGTGATTTTTTTTTAAGAGTATGGAAGG
GTTCTCAGACCTAAGAGATTGAGAAATGCTGATGTAATGTTTTAT
TATAAAGGTGTACCATGAATTATGTACCTTACTTCATATTGTTGG
ACATTAAAGTTGCTTTCAGTTTTTTTGTTTTAAA

### DAAM1

DAAM1-NRG1 fusion sequence
SEQ ID NO: 298
GAAGGAAACTGTTTAACCGGATCCCATTGTACCCAGAGTGCAGAG
CCGCCTTTCCAGCATGCAGGGGCTGCTCAGGACAGAGAGGGAGGA
GGCGCGCGGGGACGGGGACGCCCAGGAGGACCCACTCGCGGGTCC
CGCTCCGCTCCGGCA

SEQ ID NO:299=DAAM1 Exon 1
SEQ ID NO: 300=DAAM1 Exon 2
SEQ ID NO:301=DAAM1 Exon 3
SEQ ID NO:302=DAAM1 Exon 4
SEQ ID NO:303=DAAM1 Exon 5
SEQ ID NO:304=DAAM1 Exon 6
SEQ ID NO:305=DAAM1 Exon 7
SEQ ID NO:306=DAAM1 Exon 8
SEQ ID NO:307=DAAM1 Exon 9
SEQ ID NO:308=DAAM1 Exon 10
SEQ ID NO: 309=DAAM1 Exon 11
SEQ ID NO:310=DAAM1 Exon 12
SEQ ID NO:311=DAAM1 Exon 13
SEQ ID NO:312=DAAM1 Exon 14
SEQ ID NO:313=DAAM1 Exon 15
SEQ ID NO:314=DAAM1 Exon 16
SEQ ID NO:315=DAAM1 Exon 17
SEQ ID NO:316=DAAM1 Exon 18
SEQ ID NO:317=DAAM1 Exon 19
SEQ ID NO:318=DAAM1 Exon 20
SEQ ID NO:319=DAAM1 Exon 21
SEQ ID NO:320=DAAM1 Exon 22
SEQ ID NO:321=DAAM1 Exon 23
SEQ ID NO: 322=DAAM1 Exon 24
SEQ ID NO:323=DAAM1 Exon 25
SEQ ID NO:324=DAAM1 Exons 1-25

DAAM1 sequence information, providing nucleotide sequence of exons 1-25 below as indicated by alternating underlining, starting with exon 1 underlined, exon 2 not underlined, exon 3 underlined, exon 4 not underlined, and so forth.

SEQ ID NO: 324
GCGAGTTAGTAACCTACGAGCGGCTGTGAAGGAAACTGTTTAACC
GGATCCCATTGTACCCAGAGTGCAGAGCCGCCTTTCCAGCATGCA
GGGGCTGCTCAGCGTTTAGTCACATCAAGAAATAGAACAGAATTC
AGCCATGGCCCCAAGAAAGAGAGGTGGACGAGGTATTTCATTCAT
CTTTTGCTGTTTCCGAAATAATGATCACCCAGAAATCACGTATCG
GCTGCGAAATGATAGCAACTTTGCGCTTCAGACCATGGAACCAGC
ATTGCCCATGCCCCCTGTGGAGGAGCTGGATGTCATGTTCAGTGA
ACTGGTGGATGAACTGGACCTCACAGACAAACACAGAGAAGCCAT
GTTTGCACTTCCAGCTGAGAAAAAATGGCAAATATACTGTAGCAA
GAAAAAGGACCAGGAAGAAAACAAGGGAGCTACAAGTTGGCCTGA
ATTCTACATTGATCAGCTCAATTCCATGGCTGCTAGAAAATCTCT
GCTGGCTTTAGAGAAGGAAGAAGAAGAAGAAAGAAGTAAAACTAT
AGAGAGTTTAAAGACAGCACTGAGGACAAAACCAATGAGGTTTGT
AACCAGATTCATCGACTTGGATGGCCTATCATGTATCCTCAACTT
TCTAAAGACCATGGACTACGAGACCTCAGAGTCTCGAATACATAC
TTCTCTCATTGGCTGTATAAAGGCGTTAATGAACAACTCTCAAGG
CCGGGCTCACGTCCTGGCTCATTCTGAGAGTATTAATGTAATTGC
TCAGAGTCTGAGCACAGAGAACATTAAAACGAAGGTGGCCGTGCT
GGAAATCTTGGGCGCCGTGTGCCTGGTTCCCGGGGGCCACAAGAA
GGTTCTGCAGGCCATGCTGCACTACCAGAAGTATGCCAGCGAAAG
GACCCGCTTTCAGACATTAATTAACGACTTGGATAAAAGCACTGG
GCGGTATCGAGATGAAGTGAGTCTCAAGACTGCCATCATGTCCTT
CATTAATGCAGTGCTCAGCCAAGGTGCAGGAGTGGAGAGTTTGGA
CTTTAGACTTCATCTTCGCTATGAATTTCTGATGTTAGGAATTCA
ACCTGTAATAGATAAATTAAGGGAACACGAAAATTCAACATTAGA
TAGGCATTTAGACTTTTTTGAAATGCTCCGAAATGAAGATGAACT
AGAATTTGCCAAAAGATTTGAACTGGTTCACATAGACACAAAAAG
TGCAACTCAGATGTTTGAGCTGACCAGGAAGAGGCTGACACATAG
TGAAGCTTACCCGCATTTCATGTCCATCCTGCACCACTGCCTCCA
AATGCCTTACAAGAGGAGTGGCAACACTGTTCAGTACTGGCTACT
ACTAGATAGAATTATACAGCAGATAGTTATCCAGAATGACAAAGG
ACAGGACCCTGACTCCACACCTTTGGAAAACTTTAATATTAAGAA
TGTCGTACGAATGTTGGTTAATGAAAATGAAGTTAAGCAGTGGAA
AGAACAAGCGGAAAAAATGAGAAAAGAGCACAATGAGCTACAACA
GAAACTGGAAAAGAAAGAACGAGAATGTGATGCTAAGACTCAAGA
GAAGGAAGAGATGATGCAGACCTTAAATAAAATGAAAGAGAAACT
TGAAAAGGAGACTACTGAGCATAAGCAAGTCAAGCAGCAGGTGGC
GGACCTCACAGCACAGCTCCATGAGCTCAGCAGGAGGGCCGTCTG
TGCTTCAATCCCAGGTGGACCCTCGCCTGGAGCACCAGGAGGGCC
CTTTCCTTCCTCTGTGCCTGGATCTCTCCTTCCTCCCCCACCACC
CCCACCTCTACCAGGTGGGATGCTTCCCCCTCCACCGCCTCCCCT
CCCTCCAGGTGGCCCTCCTCCTCCCCCAGGGCCTCCTCCCTTAGG
GGCAATCATGCCACCTCCTGGTGCTCCAATGGGCCTAGCACTGAA
GAAGAAAAGCATTCCTCAGCCCACAAATGCCCTGAAATCCTTCAA
CTGGTCTAAACTGCCCGAGAACAAACTGGAAGGAACAGTATGGAC
CGAAATTGATGATACAAAAGTCTTCAAAATTCTAGATCTTGAAGA
CCTGGAAAGAACCTTCTCTGCCTATCAAAGACAGCAGAAAGAAGC
AGATGCCATTGATGACACTCTGAGTTCCAAACTTAAAGTTAAAGA
GCTTTCGGTGATTGATGGTCGGAGAGCTCAGAATTGCAACATCCT
TCTATCGAGGTTGAAATTATCCAATGACGAAATCAAACGGGCAAT
TCTAACAATGGACGAACAGGAAGATCTGCCCAAGGACATGTTGGA
ACAGCTCTTGAAATTTGTTCCTGAAAAAAGTGACATTGACCTATT
GGAGGAACATAAACACGAACTGGATCGGATGGCCAAGGCTGATAG
GTTCCTTTTTGAGATGAGCCGAATTAATCACTATCAGCAAAGGTT
GCAATCGCTGTACTTCAAAAAGAAGTTTGCAGAGCGTGTGGCAGA
AGTGAAACCTAAAGTGGAAGCAATTCGTTCTGGCTCAGAAGAGGT
GTTTAGGAGTGGTGCCCTCAAGCAGTTGCTGGAGGTGGTTTTGGC
ATTTGGAAATTATATGAATAAAGGTCAAAGAGGGAATGCATATGG
ATTCAAGATATCTAGCCTAAACAAAATTGCTGACACAAAATCCAG
CATCGACAAAAACATTACCCTTTTGCACTATCTCATCACTATTGT
GGAAAATAAGTACCCCAGTGTTCTCAATCTAAATGAAGAATTGCG
AGATATTCCTCAAGCTGCGAAAGTAAACATGACTGAGCTGGACAA
AGAAATAAGTACCTTGAGAAGTGGCTTGAAAGCAGTAGAGACAGA
GCTGGAATATCAGAAGTCTCAGCCCCCACAGCCCGGAGATAAGTT
TGTGTCTGTTGTCAGCCAGTTCATCACAGTAGCCAGCTTCAGCTT
CTCTGATGTTGAAGACCTTCTAGCAGAAGCTAAAGACCTGTTTAC
TAAAGCAGTGAAGCACTTTGGGGAAGAGGCTGGCAAAATACAACC
AGATGAGTTCTTTGGCATTTTTGATCAATTTCTTCAAGCTGTGTC
AGAAGCCAAACAAGAAAACGAAAATATGAGAAAGAAAAAGGAGGA
AGAAGAACGTCGAGCTCGCATGGAAGCTCAGCTCAAAGAACAACG
TGAAAGGGAACGTAAAATGAGAAAAGCTAAAGAGAATAGTGAAGA
AAGCGGAGAGTTTGATGACCTTGTTTCAGCTTTACGCTCAGGAGA
AGTGTTTGACAAAGACCTTTCTAAATTGAAACGGAATCGCAAACG
TATTACCAACCAGATGACTGACAGCAGCAGAGAGAGACCAATCAC
AAAACTTAATTTCTAATTTTCCATGAATACTTTTTTTTAGAAAGC
TCATTAGCAGCCCTCTAAAGTGACTAGAACGTTTCATTACACTGC
CTTGCAATCCAAACAGTGGCAATTTTTTCCTTCATCTGTGAGTGA
ATGTGTGAACGTGTGTATGTAAATGTATGTGTGTATATATTAAAA
AATGTATATAGATGTCTGAGTGTTGTCTGGAGACCTATACGTATG
GTTAAAAAGATTTATGTTAATGTATGTGCTCCAAAACCTTTCGTG
TATGCATTCACATTGAGTGTGGCTCATTTTCTTTCCCCGAACGCC
ATGACTGTTCAGAAGCACAATACTATCTCCTGAAAGAGATAAGAG
ACATTCCCTAGATTCAAAGGCAAAACAGAAGAAACAAACAAACAA
ACAAAAAAAGCTTGCAAAATATTTTATGGTTTCCAAGCTTGATAT
CCTTTAAAATTATTTTCATTGATGGAACTGGAGTTGTTGGAAAAA
CATAGATTTAAAATGATTTTTGATAGCTGACATTGTGATGTTGAT
GTATCACATCAGTAATAGGACCAGCTTTGAATTTCTGACATTGGT
GTGGGGATACAGTCTGTAAATGTTTATTGAGAACATCTTGCACAC
AATTTGAATTATGTAGAATGTCAATCAAGTTTTTGTATATTTAAA
AGTTGGACATCAATTTTTTCCCCTGATTTCATCAAGTTATCTCTG
CCAAGTGCTCTTGATAATTTCTTCAGATTTTTGGAAAAAAACACT
ATATAAATGCAATCCATGCTTTTTTTAAAGAACAACATTGCCAGA
GTATGCTTGTTCTAACAATATAGATATATAAACCTTAAAAATAAT
AAAATATCTCACCCAAGACTTAAAGGAAGAATTCTCTGAAGGGAT
AAAGATTACTAAAAAAAAAAAAAAAAAAAAAAAATTAATGGGGTG
CCTTTTTGTTATAGTTTCTATTTTCTGTTTTGTAGGACAAGCTGC
ATTTTCTGTAAATATAGGTCTGGACTAAAGGATACATAAAGAATG
CACAAAATGTCAACATCAGCAGAGATGCCCAGATCTATTTATCTC
TAAGTATATTTGAAGTGATTGCTGTTTATATGTTGTCATTTTAAA
ATTGTGTGTCAGTAAAGCTACCTGTAAAATTTCAGTCCAAAAAAA
TAAAGCTCTCAGGGAGACATGAATAAAATCAATGAACATTAGAAA
ATAAAATATAGATGCTTACCATTAACCTACCAACTCTTAATATCC
TTAAATTATGTGATATATAAAGAGGACTGTTACTTTTTTACTTTT
TTTTTTTTTTTTTTTTTTGGCTTTGCTTTATTTATTTGTAGTTGG
GGGCTAACGTTTTCTCTTTTCTTTCTATTGATCCTGTTGTGGTTG
GGTTTCCTGTGGAGAGAGTAGTTTGTCCTGTTGCACTAGAACATT
ATTTACTCACTAAATTGAGTTTTTCAGTCAATTAACAAATATTTA
TTAAGTTCCTACTATGTACCAGGCATAGTAGGGCTACAATGGTAA
GCAAGACAGAGTCCCTGCCCCCAAAGAGCTTATATTCTAATGGGG
ATATTAAGGGATGAATAGAATACCAATGTGTGCACTGTACAGGAA
TCATACTATTTAAAAATAATTTGTATAAACTATAATGCTTAGCAC
AGATGGCGAGTTATCTGTGCTATGTGAAAGCTGTGAAATAGTGCT
CTAAGAGTTGTCAAGAGCTGTGGTTTTACATCTTTTCCTCATTGC
AAATTTAGTGACTTTCTACACACTATATGGAAATAAATGACTAGC
AAATAAAACAGTCATAAATACAAAGCAGAGGTTGCACTCCCCCAA
TCCCGAGTTAACCCAGGTCTGCAATAACACCATGTTAAAGGTGCA
GATAGAGACTTGGCTCAAAAAGGCTTGGAGATGAGGAAGATTGGA
ATATAATGATGGTTTTGTCTGTTCCTTAACTAAAGTGCCTCTATG
TATATTCTTTTCTATTTGTAGCAGGATAAATTTGGTCTGGCTCAG
TTTTGGAACTGTATTTTGAAAATGGCTTTGTCTTACAGTTTAAGG
AATAGACAGGTGGAGGGAAAGTCACATAAAGGAGCAAGTTTGTGT
AGCTGTCCCTCTTGCCCCTTTTAATCATCCTCCTTTGATATGGCC
ATCCTGGTGGGCCTCCTTTGCCATTTCCATTTTTGGTTTCTTTCC
CTGAAAACTGTGTGCAGGTAATTCCATGTGCCATTGTTGAAAAGA
AAAAAAAAAAACAAAAAAAAAACCTACTTTTTAGATTGGTGCTGG
TGTAAGTAGCCACTTTTCTCTCTTGGGTGTGTATTTTAAACTTTT
TTTGTTTTTTTAAATTAATGCCAAAAAGAAAATGCATAATTTGTA
AACTTAATTATATGTCTTATATCTTATTAGCTTAGTAGTTGGAAC
CACTTAGTCTTTAGGTGCAAGACTGTTGTTAGATAGTACTGAGAA
AAAAAAAGTATGTGTTATGAGACTGTACATGTTTTTTTAAAAATA
GCAATATGCAATAAAGAGATGAATTCATTGGGTGTA

### ASPH

ASPH-NRG1 fusion sequence
SEQ ID NO: 325
AAGGTCTCTTCCTGCCTGAGGATGAAAACCTGAGGGAAAAAGGGG
ACTGGAGCCAGTTCACGCTGTGGCAGCAAGCCTTGCCTCCCCGAT
TGAAAGAGATGAAAAGCCAGGAATCGGCTGCAGGTTCCAAACTAG
TCCTTCGGTGTGAAA

SEQ ID NO:326=ASPH Exon 1
SEQ ID NO: 327=ASPH Exon 2
SEQ ID NO: 328=ASPH Exon 3
SEQ ID NO:329=ASPH Exon 4
SEQ ID NO:330=ASPH Exon 5
SEQ ID NO: 331=ASPH Exon 6
SEQ ID NO: 332=ASPH Exon 7
SEQ ID NO: 333=ASPH Exon 8
SEQ ID NO: 334=ASPH Exon 9
SEQ ID NO: 335=ASPH Exon 10
SEQ ID NO: 336=ASPH Exon 11
SEQ ID NO: 337=ASPH Exon 12
SEQ ID NO: 338=ASPH Exon 13
SEQ ID NO: 339=ASPH Exon 14
SEQ ID NO: 340=ASPH Exon 15
SEQ ID NO: 341=ASPH Exon 16
SEQ ID NO: 342=ASPH Exon 17
SEQ ID NO: 343=ASPH Exon 18
SEQ ID NO: 344=ASPH Exon 19
SEQ ID NO:345=ASPH Exon 20
SEQ ID NO: 346=ASPH Exon 21
SEQ ID NO: 347=ASPH Exon 22
SEQ ID NO: 348=ASPH Exon 23
SEQ ID NO: 349=ASPH Exon 24
SEQ ID NO: 350=ASPH Exon 25
SEQ ID NO: 351=Exons 1-25 ASPH
SEQ ID NO: 352=Exon 1-22 ASPH

ASPH sequence information, providing nucleotide sequence of exons 1-25 below as indicated by alternating underlining, starting with exon 1 underlined, exon 2 not underlined, exon 3 underlined, exon 4 not underlined, and so forth.

SEQ ID NO: 351
AGTCTCAAGCTCTGGTAGGCAAGTGCATGCAGTGTGCCTAAAACC
TGCCAGCAGTACTTTTGAGTTTTTTTTTTTGTTTTGTTTTACTTT
AGCATTTATTATTCATGGATTGAAGAAATCAAAATGGCTGAAGAT
AAAGAGACAAAGCATGGAGGACACAAGAATGGGAGGAAAGGCGGA
CTCTCAGGAACTTCATTCTTCACGTGGTTTATGGTGATTGCATTG
CTGGGCGTCTGGACATCTGTAGCTGTCGTTTGGTTTGATCTTGTT
GACTATGAGGAAGTTCTAGGAAAACTAGGAATCTATGATGCTGAT
GGTGATGGAGATTTTGATGTGGATGATGCCAAAGTTTTATTAGGA
CTTAAAGAGAGATCTACTTCAGAGCCAGCAGTCCCGCCAGAAGAG
GCTGAGCCACACACTGAGCCCGAGGAGCAGGTTCCTGTGGAGGCA
GAACCCCAGAATATCGAAGATGAAGCAAAAGAACAAATTCAGTCC
CTTCTCCATGAAATGGTACACGCAGAACATGTTGAGGGAGAAGAC
TTGCAACAAGAAGATGGACCCACAGGAGAACCACAACAAGAGGAT
GATGAGTTTCTTATGGCGACTGATGTAGATGATAGATTTGAGACC
CTGGAACCTGAAGTATCTCATGAAGAAACCGAGCATAGTTACCAC
GTGGAAGAGACAGTTTCACAAGACTGTAATCAGGATATGGAAGAG
ATGATGTCTGAGCAGGAAAATCCAGATTCCAGTGAACCAGTAGTA
GAAGATGAAAGATTGCACCATGATACAGATGATGTAACATACCAA
GTCTATGAGGAACAAGCAGTATATGAACCTCTAGAAAATGAAGGG
ATAGAAATCACAGAAGTAACTGCTCCCCCTGAGGATAATCCTGTA
GAAGATTCACAGGTAATTGTAGAAGAAGTAAGCATTTTTCCTGTG
GAAGAACAGCAGGAAGTACCACCAGAAACAAATAGAAAAACAGAT
GATCCAGAACAAAAAGCAAAAGTTAAGAAAAAGAAGCCTAAACTT
TTAAATAAATTTGATAAGACTATTAAAGCTGAACTTGATGCTGCA
GAAAAACTCCGTAAAAGGGGAAAAATTGAGGAAGCAGTGAATGCA
TTTAAAGAACTAGTACGCAAATACCCTCAGAGTCCACGAGCAAGA
TATGGGAAGGCGCAGTGTGAGGATGATTTGGCTGAGAAGAGGAGA
AGTAATGAGGTGCTACGTGGAGCCATCGAGACCTACCAAGAGGTG
GCCAGCCTACCTGATGTCCCTGCAGACCTGCTGAAGCTGAGTTTG
AAGCGTCGCTCAGACAGGCAACAATTTCTAGGTCATATGAGAGGT
TCCCTGCTTACCCTGCAGAGATTAGTTCAACTATTTCCCAATGAT
ACTTCCTTAAAAAATGACCTTGGCGTGGGATACCTCTTGATAGGA
GATAATGACAATGCAAAGAAAGTTTATGAAGAGGTGCTGAGTGTG
ACACCTAATGATGGCTTTGCTAAAGTCCATTATGGCTTCATCCTG
AAGGCACAGAACAAAATTGCTGAGAGCATCCCATATTTAAAGGAA
GGAATAGAATCCGGAGATCCTGGCACTGATGATGGGAGATTTTAT
TTCCACCTGGGGGATGCCATGCAGAGGGTTGGGAACAAAGAGGCA
TATAAGTGGTATGAGCTTGGGCACAAGAGAGGACACTTTGCATCT
GTCTGGCAACGCTCACTCTACAATGTGAATGGACTGAAAGCACAG
CCTTGGTGGACCCCAAAAGAAACGGGCTACACAGAGTTAGTAAAG
TCTTTAGAAAGAAACTGGAAGTTAATCCGAGATGAAGGCCTTGCA
GTGATGGATAAAGCCAAAGGTCTCTTCCTGCCTGAGGATGAAAAC
CTGAGGGAAAAAGGGGACTGGAGCCAGTTCACGCTGTGGCAGCAA
GGAAGAAGAAATGAAAATGCCTGCAAAGGAGCTCCTAAAACCTGT
ACCTTACTAGAAAAGTTCCCCGAGACAACAGGATGCAGAAGAGGA
CAGATCAAATATTCCATCATGCACCCCGGGACTCACGTGTGGCCG
CACACAGGGCCCACAAACTGCAGGCTCCGAATGCACCTGGGCTTG
GTGATTCCCAAGGAAGGCTGCAAGATTCGATGTGCCAACGAGACC
AAGACCTGGGAGGAAGGCAAGGTGCTCATCTTTGATGACTCCTTT
GAGCACGAGGTATGGCAGGATGCCTCATCTTTCCGGCTGATATTC
ATCGTGGATGTGTGGCATCCGGAACTGACACCACAGCAGAGACGC
AGCCTTCCAGCAATTTAGCATGAATTCATGCAAGCTTGGGAAACT
CTGGAGAGAGGCTGCCTTTCTGGTTCCATCTCCTTGGGTGTGAGG
ATAGAATTTCGAACACCAAGAGTCAATTCCCTTGACTTGCAGCCC
GAGTAATTCAAAGCCTCCTCCTAGGGTCAGAAGACACTAAAGGGA
ATATTTGCCTCGCTGCAATTCATTTAGGAAACACCCTGCTGTGTG
TCATCTCATGACAGCACTGGTCTTCTGCCAGTATTTAAGGTGAAC
ATTTGATAGCTTCTACCTTACCAGCCAAAGATATTTTTTCCACAT
AGAATAGGTCTAATTCAATGTATAATGAGAACATATGTAGAAACT
GTGAATGGATTGCTTTAGTTTGTAATTTTTCTATGCAGTTATATT
TTTCTAGTGTAGCTAGACTATTTTGTCATCATGTACCACTACATT
TTTGTTTATTTTAATGACAAGCTGTATAAATGCTTTACTTCTAGC
TATTTAATGGTAGCATTACTGGGGAACTCAGACTTCCCTCTTTTA
ATTCTTCTTAGTAAAAGATACTCATGAAAAAAGCAGTTTTATTTT
CCTAACAAAAAAGAAAGAGCTCATTATGTCAGTGTCTATGAACTG
TACCCATCCCAACTCTCAAATCGTTTGGTTTTTTTTATCTTGATT
GAGATCCTCTTCTCACTATGCTAGTGGTGGAGATATTGACAAAAT
CCTATTTCTTTCAAAGAGGAACTTTTCACACCGAAAAAAGAGCAT
GGAATTATTTTATATTGTTATAAAAATCCCAGATGCAAATTTTTT
TAATGCCAATTATTAGAGCTTCTGGGGAAAAAGTATAGTTCACGG
AAATAAAACTATGTTCTTTCAGGGTTGGGTGGATAGGTGGCTGCT
AGGGTGTCTGGCTCCTGGCGGCTTTGCCATCCATGAGGCAAGGGC
TGGGAACACAGTGTCTTTGCCTATGGTAGATCCATGTGAATGTCA
GGAAGCCAGCTCTTCAGTCTTGGAGATGATTTCTGCTACAATTCT
GTAGAAAGATTAAGGATGGCAGAGTAAAAGGTTACCAAGAATGCC
AGGATGTTTTTCTTGGGCGTAGGAGGTCCAGATTACTTTCCTTTT
TGATGAAAGAGTTTGGAAGACTGTCCCATCTCTCTGGCTTGAGAA
ATCTCTGCCATTTTAAACATCACTGTGAAATAGCAATTATTATCA
TCTGTATTTAGTTTTAACATTACCCACAACATAGAAATAATAGGT
AAAAATCGTCTTGCCTACTCATTCCAAAGATGATCAAGTCATTAA
TCTAGCAAAGTATTCATGTATCAGATTTTGTATATTTTGAATCAA
AGCTAACTAGGAATGTTAGATATAAGAATGTAATGATATTCATGC
ACTGAATTCTAAGCCAATATGAACAAAAATGCTGCATGAATGGCA
CATATAGGTCACCAAAGTTCATTCACAGGTAGAAAAAACTTGTGC
TTTCTTTTCCATCTAAAAACAAAAGGAGACTTTCTTTATCTCATT
TAAAGAACAGCTCTTTGAAATTGAAATTGACCCTTTTTGCTTGAC
CTTAAGGAGATTAGCTTCCAGTAGATGAGTTTGCAAAATACTTTT
CCTGTTCTTTTGTTTTGCTGGTATTGAAAACATCCCACTAAATCA
GATGAAGAGGCATGGGAGGAAAAATATCCAAATTAATTACTAAAA
TCGAGAAGAGAAGGCAAACTCTTGAAAAGTAAAAAGGTGTTTGTG
ACCTTCAGTATTTATTGAACAGAGGAAATAACTGACAAGGGCAAT
ACAATTCAATGTTCATGTAGTAACATTCATGTCACTTGTTGAATT
TGGTTCTCATATGTATATTGCATACACATAAATTCAAACTATAAG
TCGTCATTTTTGAGCCATCATCTTACATTCATGTAATGAAATTAT
GGAAGAGAGTAAAAACTAGCTCTTAACTTAGTAAATATAATATGG
TATTTAAAATCAGGTCACTACAGTAAGGTTCTAAGTATTGCCAAT
TGAAAAGCTAGAAATGGTATTACTGTTGCAAAGTGTTGTCAATAA
TTGACTCCAATAGCATTGTAAATACTTGTATCCCACAACTATTTT
AAACCCAAGCAATAAAATGGATTTTCTAATTCACTTCAATTCTTT
ATTTCTCTTACCTATCTATGTCTTGGTACATAAGAAAAGTGTTTC
CATCACTGCATTGGTAGAATTATTTCAGTGTTATTATTTTTGTGA
TTTCGTATGTCTACACAAAAATGAATTAGTTTAATTTATATTGTG
ATACAGTTGTTTGAGAAATATTTTTAATTCTGTTTCAACTTTATT
TCTCCAAGTGTATAATATAAAAATTACTTCTGTTATTGTTCTCTA
CCAATAGGGGAAAAAATTAAAACATTAGATCCATTGAGAAAGAGA
TGATGTAATAAATAATTAAGATTAGTAATAATATTATCAGGGGTG
ATTATGACCAGTTGAATAATCTCTTTCCCTTGAATTATTTAGCTA
ACAAATTAACTCTCCCAAATATTTAAAATAATGTAAAATCATATT
TTACTGCCCATTATTAACTAAAATATTTTTGTTTGACTTTGAGCA
CCAACTGGTAATACTAATAAATACCCATGTCATGCAGATGGCTGG
GCGAATAAGAGATGTCTAAAAATATGCACTGGTCTTGGAAAACAT
GGCACAAGTAAGGATATCATATATGATGTCTGTTTATTTTATGTC
TGATTTCTTTTGAATGAGTAGTTGGGGACTCCATTTCTAAGGAGA
CTAGGTAAATAAAATGACCTTTGACATTTCA

### NOTCH2

NOTCH2-NRG1 fusion sequence
SEQ ID NO: 353
CCTCCTGTACTCCAGGCTCCACCTGCATCGACCGTGTGGCCTCCT
TCTCTTGCATGTGCCCAGAGGGGAAGGCAGCTACATCTACATCCA
CCACTGGGACAAGCCATCTTGTAAAATGTGCGGAGAAGGAGAAAA
CTTTCTGTGTGAATG

SEQ ID NO:354=NOTCH2 Exon 1
SEQ ID NO: 355=NOTCH2 Exon 2
SEQ ID NO: 356=NOTCH2 Exon 3
SEQ ID NO: 357=NOTCH2 Exon 4
SEQ ID NO:358=NOTCH2 Exon 5
SEQ ID NO: 359=NOTCH2 Exon 6
SEQ ID NO: 360=NOTCH2 Exon 7
SEQ ID NO:361=NOTCH2 Exon 8-34
SEQ ID NO:362=NOTCH2 Exons 1-34
SEQ ID NO:363=NOTCH2 Exons 1-6

NOTCH2 sequence information, providing nucleotide sequence of exons 1-34 below as indicated by alternating underlining, starting with exon 1 underlined, exon 2 not underlined, exon 3 underlined, exon 4 not underlined, exon 5 underlined, exon 6 not underlined, exon 7 underlined and exons 8-34 not underlined.

SEQ ID NO: 362
AGGCTGCTTCGTTGCACACCCGAGAAAGTTTCAGCCAAACTTCGGGCGGCGGCTGAGGCGGCGGC
CGAGGAGCGGCGGACTCGGGGCGCGGGGAGTCGAGGCATTTGCGCCTGGGCTTCGGAGCGTAGC
GCCAGGGCCTGAGCCTTTGAAGCAGGAGGAGGGGAGGAGAGAGTGGGGCTCCTCTATCGGGACCC
CCTCCCCATGTGGATCTGCCCAGGCGGCGGCGGCGGCGGCGGAGGAGGAGGCGACCGAGAAGAT
GCCCGCCCTGCGCCCCGCTCTGCTGTGGGCGCTGCTGGCGCTCTGGCTGTGCTGCGCGGCCCCCG
CGCATGCATTGCAGTGTCGAGATGGCTATGAACCCTGTGTAAATGAAGGAATGTGTGTTACCTACCA
CAATGGCACAGGATACTGCAAATGTCCAGAAGGCTTCTTGGGGGAATATTGTCAACATCGAGACCCC
TGTGAGAAGAACCGCTGCCAGAATGGTGGGACTTGTGTGGCCCAGGCCATGCTGGGGAAAGCCAC
GTGCCGATGTGCCTCAGGGTTTACAGGAGAGGACTGCCAGTACTCAACATCTCATCCATGCTTTGTG
TCTCGACCCTGCCTGAATGGCGGCACATGCCATATGCTCAGCCGGGATACCTATGAGTGCACCTGTC
AAGTCGGGTTTACAGGTAAGGAGTGCCAATGGACGGATGCCTGCCTGTCTCATCCCTGTGCAAATG
GAAGTACCTGTACCACTGTGGCCAACCAGTTCTCCTGCAAATGCCTCACAGGCTTCACAGGGCAGAA
ATGTGAGACTGATGTCAATGAGTGTGACATTCCAGGACACTGCCAGCATGGTGGCACCTGCCTCAAC
CTGCCTGGTTCCTACCAGTGCCAGTGCCCTCAGGGCTTCACAGGCCAGTACTGTGACAGCCTGTATG
TGCCCTGTGCACCCTCACCTTGTGTCAATGGAGGCACCTGTCGGCAGACTGGTGACTTCACTTTTGA
GTGCAACTGCCTTCCAGGTTTTGAAGGGAGCACCTGTGAGAGGAATATTGATGACTGCCCTAACCAC
AGGTGTCAGAATGGAGGGGTTTGTGTGGATGGGGTCAACACTTACAACTGCCGCTGTCCCCCACAA
TGGACAGGACAGTTCTGCACAGAGGATGTGGATGAATGCCTGCTGCAGCCCAATGCCTGTCAAAAT
GGGGGCACCTGTGCCAACCGCAATGGAGGCTATGGCTGTGTATGTGTCAACGGCTGGAGTGGAGAT
GACTGCAGTGAGAACATTGATGATTGTGCCTTCGCCTCCTGTACTCCAGGCTCCACCTGCATCGACC
GTGTGGCCTCCTTCTCTTGCATGTGCCCAGAGGGGAAGGCAGGTCTCCTGTGTCATCTGGATGATG
CATGCATCAGCAATCCTTGCCACAAGGGGGCACTGTGTGACACCAACCCCCTAAATGGGCAATATAT
TTGCACCTGCCCACAAGGCTACAAAGGGGCTGACTGCACAGAAGATGTGGATGAATGTGCCATGGC
CAATAGCAATCCTTGTGAGCATGCAGGAAAATGTGTGAACACGGATGGCGCCTTCCACTGTGAGTGT
CTGAAGGGTTATGCAGGACCTCGTTGTGAGATGGACATCAATGAGTGCCATTCAGACCCCTGCCAGA
ATGATGCTACCTGTCTGGATAAGATTGGAGGCTTCACATGTCTGTGCATGCCAGGTTTCAAAGGTGT
GCATTGTGAATTAGAAATAAATGAATGTCAGAGCAACCCTTGTGTGAACAATGGGCAGTGTGTGGAT
AAAGTCAATCGTTTCCAGTGCCTGTGTCCTCCTGGTTTCACTGGGCCAGTTTGCCAGATTGATATTGA
TGACTGTTCCAGTACTCCGTGTCTGAATGGGGCAAAGTGTATCGATCACCCGAATGGCTATGAATGC
CAGTGTGCCACAGGTTTCACTGGTGTGTTGTGTGAGGAGAACATTGACAACTGTGACCCCGATCCTT
GCCACCATGGTCAGTGTCAGGATGGTATTGATTCCTACACCTGCATCTGCAATCCCGGGTACATGGG
CGCCATCTGCAGTGACCAGATTGATGAATGTTACAGCAGCCCTTGCCTGAACGATGGTCGCTGCATT
GACCTGGTCAATGGCTACCAGTGCAACTGCCAGCCAGGCACGTCAGGGGTTAATTGTGAAATTAATT
TTGATGACTGTGCAAGTAACCCTTGTATCCATGGAATCTGTATGGATGGCATTAATCGCTACAGTTGT
GTCTGCTCACCAGGATTCACAGGGCAGAGATGTAACATTGACATTGATGAGTGTGCCTCCAATCCCT
GTCGCAAGGGTGCAACATGTATCAACGGTGTGAATGGTTTCCGCTGTATATGCCCCGAGGGACCCC
ATCACCCCAGCTGCTACTCACAGGTGAACGAATGCCTGAGCAATCCCTGCATCCATGGAAACTGTAC
TGGAGGTCTCAGTGGATATAAGTGTCTCTGTGATGCAGGCTGGGTTGGCATCAACTGTGAAGTGGA
CAAAAATGAATGCCTTTCGAATCCATGCCAGAATGGAGGAACTTGTGACAATCTGGTGAATGGATAC
AGGTGTACTTGCAAGAAGGGCTTTAAAGGCTATAACTGCCAGGTGAATATTGATGAATGTGCCTCAA
ATCCATGCCTGAACCAAGGAACCTGCTTTGATGACATAAGTGGCTACACTTGCCACTGTGTGCTGCC
ATACACAGGCAAGAATTGTCAGACAGTATTGGCTCCCTGTTCCCCAAACCCTTGTGAGAATGCTGCT
GTTTGCAAAGAGTCACCAAATTTTGAGAGTTATACTTGCTTGTGTGCTCCTGGCTGGCAAGGTCAGC
GGTGTACCATTGACATTGACGAGTGTATCTCCAAGCCCTGCATGAACCATGGTCTCTGCCATAACAC
CCAGGGCAGCTACATGTGTGAATGTCCACCAGGCTTCAGTGGTATGGACTGTGAGGAGGACATTGA
TGACTGCCTTGCCAATCCTTGCCAGAATGGAGGTTCCTGTATGGATGGAGTGAATACTTTCTCCTGC
CTCTGCCTTCCGGGTTTCACTGGGGATAAGTGCCAGACAGACATGAATGAGTGTCTGAGTGAACCCT
GTAAGAATGGAGGGACCTGCTCTGACTACGTCAACAGTTACACTTGCAAGTGCCAGGCAGGATTTGA
TGGAGTCCATTGTGAGAACAACATCAATGAGTGCACTGAGAGCTCCTGTTTCAATGGTGGCACATGT
GTTGATGGGATTAACTCCTTCTCTTGCTTGTGCCCTGTGGGTTTCACTGGATCCTTCTGCCTCCATGA
GATCAATGAATGCAGCTCTCATCCATGCCTGAATGAGGGAACGTGTGTTGATGGCCTGGGTACCTAC
CGCTGCAGCTGCCCCCTGGGCTACACTGGGAAAAACTGTCAGACCCTGGTGAATCTCTGCAGTCGG
TCTCCATGTAAAAACAAAGGTACTTGCGTTCAGAAAAAAGCAGAGTCCCAGTGCCTATGTCCATCTG
GATGGGCTGGTGCCTATTGTGACGTGCCCAATGTCTCTTGTGACATAGCAGCCTCCAGGAGAGGTG
TGCTTGTTGAACACTTGTGCCAGCACTCAGGTGTCTGCATCAATGCTGGCAACACGCATTACTGTCA
GTGCCCCCTGGGCTATACTGGGAGCTACTGTGAGGAGCAACTCGATGAGTGTGCGTCCAACCCCTG
CCAGCACGGGGCAACATGCAGTGACTTCATTGGTGGATACAGATGCGAGTGTGTCCCAGGCTATCA
GGGTGTCAACTGTGAGTATGAAGTGGATGAGTGCCAGAATCAGCCCTGCCAGAATGGAGGCACCTG
TATTGACCTTGTGAACCATTTCAAGTGCTCTTGCCCACCAGGCACTCGGGGCCTACTCTGTGAAGAG
AACATTGATGACTGTGCCCGGGGTCCCCATTGCCTTAATGGTGGTCAGTGCATGGATAGGATTGGA
GGCTACAGTTGTCGCTGCTTGCCTGGCTTTGCTGGGGAGCGTTGTGAGGGAGACATCAACGAGTGC
CTCTCCAACCCCTGCAGCTCTGAGGGCAGCCTGGACTGTATACAGCTCACCAATGACTACCTGTGTG
TTTGCCGTAGTGCCTTTACTGGCCGGCACTGTGAAACCTTCGTCGATGTGTGTCCCCAGATGCCCTG
CCTGAATGGAGGGACTTGTGCTGTGGCCAGTAACATGCCTGATGGTTTCATTTGCCGTTGTCCCCCG
GGATTTTCCGGGGCAAGGTGCCAGAGCAGCTGTGGACAAGTGAAATGTAGGAAGGGGGAGCAGTG
TGTGCACACCGCCTCTGGACCCCGCTGCTTCTGCCCCAGTCCCCGGGACTGCGAGTCAGGCTGTGC
CAGTAGCCCCTGCCAGCACGGGGGCAGCTGCCACCCTCAGCGCCAGCCTCCTTATTACTCCTGCCA
GTGTGCCCCACCATTCTCGGGTAGCCGCTGTGAACTCTACACGGCACCCCCCAGCACCCCTCCTGCC
ACCTGTCTGAGCCAGTATTGTGCCGACAAAGCTCGGGATGGCGTCTGTGATGAGGCCTGCAACAGC
CATGCCTGCCAGTGGGATGGGGGTGACTGTTCTCTCACCATGGAGAACCCCTGGGCCAACTGCTCC
TCCCCACTTCCCTGCTGGGATTATATCAACAACCAGTGTGATGAGCTGTGCAACACGGTCGAGTGCC
TGTTTGACAACTTTGAATGCCAGGGGAACAGCAAGACATGCAAGTATGACAAATACTGTGCAGACCA
CTTCAAAGACAACCACTGTGACCAGGGGTGCAACAGTGAGGAGTGTGGTTGGGATGGGCTGGACTG
TGCTGCTGACCAACCTGAGAACCTGGCAGAAGGTACCCTGGTTATTGTGGTATTGATGCCACCTGAA
CAACTGCTCCAGGATGCTCGCAGCTTCTTGCGGGCACTGGGTACCCTGCTCCACACCAACCTGCGC
ATTAAGCGGGACTCCCAGGGGGAACTCATGGTGTACCCCTATTATGGTGAGAAGTCAGCTGCTATGA
AGAAACAGAGGATGACACGCAGATCCCTTCCTGGTGAACAAGAACAGGAGGTGGCTGGCTCTAAAG
TCTTTCTGGAAATTGACAACCGCCAGTGTGTTCAAGACTCAGACCACTGCTTCAAGAACACGGATGC
AGCAGCAGCTCTCCTGGCCTCTCACGCCATACAGGGGACCCTGTCATACCCTCTTGTGTCTGTCGTC
AGTGAATCCCTGACTCCAGAACGCACTCAGCTCCTCTATCTCCTTGCTGTTGCTGTTGTCATCATTCT
GTTTATTATTCTGCTGGGGGTAATCATGGCAAAACGAAAGCGTAAGCATGGCTCTCTCTGGCTGCCT
GAAGGTTTCACTCTTCGCCGAGATGCAAGCAATCACAAGCGTCGTGAGCCAGTGGGACAGGATGCT
GTGGGGCTGAAAAATCTCTCAGTGCAAGTCTCAGAAGCTAACCTAATTGGTACTGGAACAAGTGAAC
ACTGGGTCGATGATGAAGGGCCCCAGCCAAAGAAAGTAAAGGCTGAAGATGAGGCCTTACTCTCAG
AAGAAGATGACCCCATTGATCGACGGCCATGGACACAGCAGCACCTTGAAGCTGCAGACATCCGTA
GGACACCATCGCTGGCTCTCACCCCTCCTCAGGCAGAGCAGGAGGTGGATGTGTTAGATGTGAATG
TCCGTGGCCCAGATGGCTGCACCCCATTGATGTTGGCTTCTCTCCGAGGAGGCAGCTCAGATTTGAG
TGATGAAGATGAAGATGCAGAGGACTCTTCTGCTAACATCATCACAGACTTGGTCTACCAGGGTGCC
AGCCTCCAGGCCCAGACAGACCGGACTGGTGAGATGGCCCTGCACCTTGCAGCCCGCTACTCACGG
GCTGATGCTGCCAAGCGTCTCCTGGATGCAGGTGCAGATGCCAATGCCCAGGACAACATGGGCCGC
TGTCCACTCCATGCTGCAGTGGCAGCTGATGCCCAAGGTGTCTTCCAGATTCTGATTCGCAACCGAG
TAACTGATCTAGATGCCAGGATGAATGATGGTACTACACCCCTGATCCTGGCTGCCCGCCTGGCTGT
GGAGGGAATGGTGGCAGAACTGATCAACTGCCAAGCGGATGTGAATGCAGTGGATGACCATGGAAA
ATCTGCTCTTCACTGGGCAGCTGCTGTCAATAATGTGGAGGCAACTCTTTTGTTGTTGAAAAATGGG
GCCAACCGAGACATGCAGGACAACAAGGAAGAGACACCTCTGTTTCTTGCTGCCCGGGAGGGGAGC
TATGAAGCAGCCAAGATCCTGTTAGACCATTTTGCCAATCGAGACATCACAGACCATATGGATCGTC
TTCCCCGGGATGTGGCTCGGGATCGCATGCACCATGACATTGTGCGCCTTCTGGATGAATACAATGT
GACCCCAAGCCCTCCAGGCACCGTGTTGACTTCTGCTCTCTCACCTGTCATCTGTGGGCCCAACAGA
TCTTTCCTCAGCCTGAAGCACACCCCAATGGGCAAGAAGTCTAGACGGCCCAGTGCCAAGAGTACCA
TGCCTACTAGCCTCCCTAACCTTGCCAAGGAGGCAAAGGATGCCAAGGGTAGTAGGAGGAAGAAGT
CTCTGAGTGAGAAGGTCCAACTGTCTGAGAGTTCAGTAACTTTATCCCCTGTTGATTCCCTAGAATCT
CCTCACACGTATGTTTCCGACACCACATCCTCTCCAATGATTACATCCCCTGGGATCTTACAGGCCTC
ACCCAACCCTATGTTGGCCACTGCCGCCCCTCCTGCCCCAGTCCATGCCCAGCATGCACTATCTTTTT
CTAACCTTCATGAAATGCAGCCTTTGGCACATGGGGCCAGCACTGTGCTTCCCTCAGTGAGCCAGTT
GCTATCCCACCACCACATTGTGTCTCCAGGCAGTGGCAGTGCTGGAAGCTTGAGTAGGCTCCATCCA
GTCCCAGTCCCAGCAGATTGGATGAACCGCATGGAGGTGAATGAGACCCAGTACAATGAGATGTTT
GGTATGGTCCTGGCTCCAGCTGAGGGCACCCATCCTGGCATAGCTCCCCAGAGCAGGCCACCTGAA
GGGAAGCACATAACCACCCCTCGGGAGCCCTTGCCCCCCATTGTGACTTTCCAGCTCATCCCTAAAG
GCAGTATTGCCCAACCAGCGGGGGCTCCCCAGCCTCAGTCCACCTGCCCTCCAGCTGTTGCGGGCC
CCCTGCCCACCATGTACCAGATTCCAGAAATGGCCCGTTTGCCCAGTGTGGCTTTCCCCACTGCCAT
GATGCCCCAGCAGGACGGGCAGGTAGCTCAGACCATTCTCCCAGCCTATCATCCTTTCCCAGCCTCT
GTGGGCAAGTACCCCACACCCCCTTCACAGCACAGTTATGCTTCCTCAAATGCTGCTGAGCGAACAC
CCAGTCACAGTGGTCACCTCCAGGGTGAGCATCCCTACCTGACACCATCCCCAGAGTCTCCTGACCA
GTGGTCAAGTTCATCACCCCACTCTGCTTCTGACTGGTCAGATGTGACCACCAGCCCTACCCCTGGG
GGTGCTGGAGGAGGTCAGCGGGGACCTGGGACACACATGTCTGAGCCACCACACAACAACATGCA
GGTTTATGCGTGAGAGAGTCCACCTCCAGTGTAGAGACATAACTGACTTTTGTAAATGCTGCTGAGG
AACAAATGAAGGTCATCCGGGAGAGAAATGAAGAAATCTCTGGAGCCAGCTTCTAGAGGTAGGAAA
GAGAAGATGTTCTTATTCAGATAATGCAAGAGAAGCAATTCGTCAGTTTCACTGGGTATCTGCAAGG
CTTATTGATTATTCTAATCTAATAAGACAAGTTTGTGGAAATGCAAGATGAATACAAGCCTTGGGTCC
ATGTTTACTCTCTTCTATTTGGAGAATAAGATGGATGCTTATTGAAGCCCAGACATTCTTGCAGCTTG
GACTGCATTTTAAGCCCTGCAGGCTTCTGCCATATCCATGAGAAGATTCTACACTAGCGTCCTGTTG
GGAATTATGCCCTGGAATTCTGCCTGAATTGACCTACGCATCTCCTCCTCCTTGGACATTCTTTTGTC
TTCATTTGGTGCTTTTGGTTTTGCACCTCTCCGTGATTGTAGCCCTACCAGCATGTTATAGGGCAAGA
CCTTTGTGCTTTTGATCATTCTGGCCCATGAAAGCAACTTTGGTCTCCTTTCCCCTCCTGTCTTCCCG
GTATCCCTTGGAGTCTCACAAGGTTTACTTTGGTATGGTTCTCAGCACAAACCTTTCAAGTATGTTGT
TTCTTTGGAAAATGGACATACTGTATTGTGTTCTCCTGCATATATCATTCCTGGAGAGAGAAGGGGA
GAAGAATACTTTTCTTCAACAAATTTTGGGGGCAGGAGATCCCTTCAAGAGGCTGCACCTTAATTTTT
CTTGTCTGTGTGCAGGTCTTCATATAAACTTTACCAGGAAGAAGGGTGTGAGTTTGTTGTTTTTCTGT
GTATGGGCCTGGTCAGTGTAAAGTTTTATCCTTGATAGTCTAGTTACTATGACCCTCCCCACTTTTTTA
AAACCAGAAAAAGGTTTGGAATGTTGGAATGACCAAGAGACAAGTTAACTCGTGCAAGAGCCAGTTA
CCCACCCACAGGTCCCCCTACTTCCTGCCAAGCATTCCATTGACTGCCTGTATGGAACACATTTGTCC
CAGATCTGAGCATTCTAGGCCTGTTTCACTCACTCACCCAGCATATGAAACTAGTCTTAACTGTTGAG
CCTTTCCTTTCATATCCACAGAAGACACTGTCTCAAATGTTGTACCCTTGCCATTTAGGACTGAACTTT
CCTTAGCCCAAGGGACCCAGTGACAGTTGTCTTCCGTTTGTCAGATGATCAGTCTCTACTGATTATCT
TGCTGCTTAAAGGCCTGCTCACCAATCTTTCTTTCACACCGTGTGGTCCGTGTTACTGGTATACCCAG
TATGTTCTCACTGAAGACATGGACTTTATATGTTCAAGTGCAGGAATTGGAAAGTTGGACTTGTTTTC
TATGATCCAAAACAGCCCTATAAGAAGGTTGGAAAAGGAGGAACTATATAGCAGCCTTTGCTATTTTC
TGCTACCATTTCTTTTCCTCTGAAGCGGCCATGACATTCCCTTTGGCAACTAACGTAGAAACTCAACA
GAACATTTTCCTTTCCTAGAGTCACCTTTTAGATGATAATGGACAACTATAGACTTGCTCATTGTTCAG
ACTGATTGCCCCTCACCTGAATCCACTCTCTGTATTCATGCTCTTGGCAATTTCTTTGACTTTCTTTTA
AGGGCAGAAGCATTTTAGTTAATTGTAGATAAAGAATAGTTTTCTTCCTCTTCTCCTTGGGCCAGTTA
ATAATTGGTCCATGGCTACACTGCAACTTCCGTCCAGTGCTGTGATGCCCATGACACCTGCAAAATA
AGTTCTGCCTGGGCATTTTGTAGATATTAACAGGTGAATTCCCGACTCTTTTGGTTTGAATGACAGTT
CTCATTCCTTCTATGGCTGCAAGTATGCATCAGTGCTTCCCACTTACCTGATTTGTCTGTCGGTGGCC
CCATATGGAAACCCTGCGTGTCTGTTGGCATAATAGTTTACAAATGGTTTTTTCAGTCCTATCCAAAT
TTATTGAACCAACAAAAATAATTACTTCTGCCCTGAGATAAGCAGATTAAGTTTGTTCATTCTCTGCTT
TATTCTCTCCATGTGGCAACATTCTGTCAGCCTCTTTCATAGTGTGCAAACATTTTATCATTCTAAATG
GTGACTCTCTGCCCTTGGACCCATTTATTATTCACAGATGGGGAGAACCTATCTGCATGGACCTCTGT
GGACCACAGCGTACCTGCCCCTTTCTGCCCTCCTGCTCCAGCCCCACTTCTGAAAGTATCAGCTACT
GATCCAGCCACTGGATATTTTATATCCTCCCTTTTCCTTAAGCACAATGTCAGACCAAATTGCTTGTTT
CTTTTTCTTGGACTACTTTAATTTGGATCCTTTGGGTTTGGAGAAAGGGAATGTGAAAGCTGTCATTA
CAGACAACAGGTTTCAGTGATGAGGAGGACAACACTGCCTTTCAAACTTTTTACTGATCTCTTAGATT
TTAAGAACTCTTGAATTGTGTGGTATCTAATAAAAGGGAAGGTAAGATGGATAATCACTTTCTCATTT
GGGTTCTGAATTGGAGACTCAGTTTTTATGAGACACATCTTTTATGCCATGTATAGATCCTCCCCTGC
TATTTTTGGTTTATTTTTATTGTTATAAATGCTTTCTTTCTTTGACTCCTCTTCTGCCTGCCTTTGGGGA
TAGGTTTTTTTGTTTGTTTATTTGCTTCCTCTGTTTTGTTTTAAGCATCATTTTCTTATGTGAGGTGGG
GAAGGGAAAGGTATGAGGGAAAGAGAGTCTGAGAATTAAAATATTTTAGTATAAGCAATTGGCTGTG
ATGCTCAAATCCATTGCATCCTCTTATTGAATTTGCCAATTTGTAATTTTTGCATAATAAAGAACCAAA
GGTGTAATGTTTTGTTGAGAGGTGGTTTAGGGATTTTGGCCCTAACCAATACATTGAATGTATGATGA
CTATTTGGGAGGACACATTTATGTACCCAGAGGCCCCCACTAATAAGTGGTACTATGGTTACTTCCTT
GTGTACATTTCTCTTAAAAGTGATATTATATCTGTTTGTATGAGAAACCCAGTAACCAATAAAATGACC
GCATATTCCTGACTAAACGTAGTAAGGAAAATGCACACTTTGTTTTTACTTTTCCGTTTCATTCTAAAG
GTAGTTAAGATGAAATTTATATGAAAGCATTTTTATCACAAAATAAAAAAGGTTTGCCAAGCTCAGTG
GTGTTGTATTTTTTATTTTCCAATACTGCATCCATGGCCTGGCAGTGTTACCTCATGATGTCATAATTT
GCTGAGAGAGCAAATTTTCTTTTCTTTCTGAATCCCACAAAGCCTAGCACCAAACTTCTTTTTTTCTTC
CTTTAATTAGATCATAAATAAATGATCCTGGGGAAAAAGCATCTGTCAAATAGGAAACATCACAAAAC
TGAGCACTCTTCTGTGCACTAGCCATAGCTGGTGACAAACAGATGGTTGCTCAGGGACAAGGTGCCT
TCCAATGGAAATGCGAAGTAGTTGCTATAGCAAGAATTGGGAACTGGGATATAAGTCATAATATTAA
TTATGCTGTTATGTAAATGATTGGTTTGTAACATTCCTTAAGTGAAATTTGTGTAGAACTTAATATACA
GGATTATAAAATAATATTTTGTGTATAAATTTGTTATAAGTTCACATTCATACATTTATTTATAAAGTCA
GTGAGATATTTGAACATGAA

### CD74

CD74-NRG1 fusion sequence
SEQ ID NO: 364
AGGGCCGGCTGGACAAACTGACAGTCACCTCCCAGAACCTGCAGCTGGA
GAACCTGCGCATGAAGCTTCCCAAGCCCTTGCCTCCCCGATTGAAAGAG
ATGAAAAGCCAGGAATCGGCTGCAGGTTCCAAACTAGTCCTTCGGTGTG
AAA

SEQ ID NO: 365=CD74 Exon 1
SEQ ID NO:366=CD74 Exon 2
SEQ ID NO:367=CD74 Exon 3
SEQ ID NO:368=CD74 Exon 4
SEQ ID NO:369=CD74 Exon 5
SEQ ID NO:370=CD74 Exon 6
SEQ ID NO:371=CD74 Exon 7
SEQ ID NO:372=CD74 Exon 8
SEQ ID NO:373=CD74 Exon 9
SEQ ID NO:374=CD74 Exons 1-9
SEQ ID NO:375=CD74 Exons 1-2

CD74 sequence information, providing nucleotide sequence of exons 1-9 below as indicated by alternating underlining, starting with exon 1 underlined, exon 2 not underlined, exon 3 underlined, exon 4 not underlined, and so forth.

SEQ ID NO: 374
ATCCTGCCCCGCAAAAGGCAGCTTCACCAAAGTGGGGTATTTCCAGCCTTTGTAGCTTTCACTTCCA
CATCTACCAAGTGGGCGGAGTGGCCTTCTGTGGACGAATCAGATTCCTCTCCAGCACCGACTTTAAG
AGGCGAGCCGGGGGGTCAGGGTCCCAGATGCACAGGAGGAGAAGCAGGAGCTGTCGGGAAGATC
AGAAGCCAGTCATGGATGACCAGCGCGACCTTATCTCCAACAATGAGCAACTGCCCATGCTGGGCC
GGCGCCCTGGGGCCCCGGAGAGCAAGTGCAGCCGCGGAGCCCTGTACACAGGCTTTTCCATCCTG
GTGACTCTGCTCCTCGCTGGCCAGGCCACCACCGCCTACTTCCTGTACCAGCAGCAGGGCCGGCTG
GACAAACTGACAGTCACCTCCCAGAACCTGCAGCTGGAGAACCTGCGCATGAAGCTTCCCAAGCCTC
CCAAGCCTGTGAGCAAGATGCGCATGGCCACCCCGCTGCTGATGCAGGCGCTGCCCATGGGAGCC
CTGCCCCAGGGGCCCATGCAGAATGCCACCAAGTATGGCAACATGACAGAGGACCATGTGATGCAC
CTGCTCCAGAATGCTGACCCCCTGAAGGTGTACCCGCCACTGAAGGGGAGCTTCCCGGAGAACCTG
AGACACCTTAAGAACACCATGGAGACCATAGACTGGAAGGTCTTTGAGAGCTGGATGCACCATTGG
CTCCTGTTTGAAATGAGCAGGCACTCCTTGGAGCAAAAGCCCACTGACGCTCCACCGAAAGTACTGA
CCAAGTGCCAGGAAGAGGTCAGCCACATCCCTGCTGTCCACCCGGGTTCATTCAGGCCCAAGTGCG
ACGAGAACGGCAACTATCTGCCACTCCAGTGCTATGGGAGCATCGGCTACTGCTGGTGTGTCTTCCC
CAACGGCACGGAGTCCCCAACACCAGAAGCCGCGGGCACCATAACTGCAGTGAGTCACTGGAACT
GGAGGACCCGTCTTCTGGGCTGGGTGTGACCAAGCAGGATCTGGGCCCAGTCCCCATGTGAGAGC
AGCAGAGGCGGTCTTCAACATCCTGCCAGCCCCACACAGCTACAGCTTTCTTGCTCCCTTCAGCCCC
CAGCCCCTCCCCCATCTCCCACCCTGTACCTCATCCCATGAGACCCTGGTGCCTGGCTCTTTCGTCA
CCCTTGGACAAGACAAACCAAGTCGGAACAGCAGATAACAATGCAGCAAGGCCCTGCTGCCCAATC
TCCATCTGTCAACAGGGGCGTGAGGTCCCAGGAAGTGGCCAAAAGCTAGACAGATCCCCGTTCCTG
ACATCACAGCAGCCTCCAACACAAGGCTCCAAGACCTAGGCTCATGGACGAGATGGGAAGGCACAG
GGAGAAGGGATAACCCTACACCCAGACCCCAGGCTGGACATGCTGACTGTCCTCTCCCCTCCAGCC
TTTGGCCTTGGCTTTTCTAGCCTATTTACCTGCAGGCTGAGCCACTCTCTTCCCTTTCCCCAGCATCA
CTCCCCAAGGAAGAGCCAATGTTTTCCACCCATAATCCTTTCTGCCGACCCCTAGTTCCCTCTGCTCA
GCCAAGCTTGTTATCAGCTTTCAGGGCCATGGTTCACATTAGAATAAAAGGTAGTAATTAGAA

### SDC4

SDC4-NRG1 fusion sequence
SEQ ID NO: 376
TACCAGACGATGAGGATGTAGTGGGGCCCGGGCAGGAATCTGATGACTT
TGAGCTGTCTGGCTCTGGAGATCTGGCCTTGCCTCCCCGATTGAAAGAG
ATGAAAAGCCAGGAATCGGCTGCAGGTTCCAAACTAGTCCTTCGGTGTG
AAA

SEQ ID NO:377=SDC4 Exon 1
SEQ ID NO:378=SDC4 Exon 2
SEQ ID NO:379=SDC4 Exon 3
SEQ ID NO:380=SDC4 Exon 4
SEQ ID NO:381=SDC4 Exon 5
SEQ ID NO:382=SDC4 Exons 1-5
SEQ ID NO:383=SDC4 Exons 1-2
SEQ ID NO:470=SDC4 Exons 1-4

SDC4 sequence information, providing nucleotide sequence of exons 1-5 below as indicated by alternating underlining, starting with exon 1 underlined, exon 2 not underlined, exon 3 underlined, exon 4 not underlined, and so forth.

SEQ ID NO: 382
ACTCGCCGCAGCCTGCGCGCCTTCTCCAGTCCGCGGTGCCATGGCCCCCGCCCGTCTGTTCGCGCT
GCTGCTGTTCTTCGTAGGCGGAGTCGCCGAGTCGATCCGAGAGACTGAGGTCATCGACCCCCAGGA
CCTCCTAGAAGGCCGATACTTCTCCGGAGCCCTACCAGACGATGAGGATGTAGTGGGGCCCGGGCA
GGAATCTGATGACTTTGAGCTGTCTGGCTCTGGAGATCTGGATGACTTGGAAGACTCCATGATCGGC
CCTGAAGTTGTCCATCCCTTGGTGCCTCTAGATAACCATATCCCTGAGAGGGCAGGGTCTGGGAGCC
AAGTCCCCACCGAACCCAAGAAACTAGAGGAGAATGAGGTTATCCCCAAGAGAATCTCACCCGTTGA
AGAGAGTGAGGATGTGTCCAACAAGGTGTCAATGTCCAGCACTGTGCAGGGCAGCAACATCTTTGA
GAGAACGGAGGTCCTGGCAGCTCTGATTGTGGGTGGCATCGTGGGCATCCTCTTTGCCGTCTTCCT
GATCCTACTGCTCATGTACCGTATGAAGAAGAAGGATGAAGGCAGCTATGACCTGGGCAAGAAACC
CATCTACAAGAAAGCCCCCACCAATGAGTTCTACGCGTGAAGCTTGCTTGTGGGCACTGGCTTGGAC
TTTAGCGGGGAGGGAAGCCAGGGGATTTTGAAGGGTGGACATTAGGGTAGGGTGAGGTCAACCTA
ATACTGACTTGTCAGTATCTCCAGCTCTGATTACCTTTGAAGTGTTCAGAAGAGACATTGTCTTCTAC
TGTTCTGCCAGGTTCTTCTTGAGCTTTGGGCCTCAGTTGCCCTGGCAGAAAAATGGATTCAACTTGG
CCTTTCTGAAGGCAAGACTGGGATTGGATCACTTCTTAAACTTCCAGTTAAGAATCTAGGTCCGCCCT
CAAGCCCATACTGACCATGCCTCATCCAGAGCTCCTCTGAAGCCAGGGGGCTAACGGATGTTGTGT
GGAGTCCTGGCTGGAGGTCCTCCCCCAGTGGCCTTCCTCCCTTCCTTTCACAGCCGGTCTCTCTGCC
AGGAAATGGGGGAAGGAACTAGAACCACCTGCACCTTGAGATGTTTCTGTAAATGGGTACTTGTGAT
CACACTACGGGAATCTCTGTGGTATATACCTGGGGCCATTCTAGGCTCTTTCAAGTGACTTTTGGAAA
TCAACCTTTTTTATTTGGGGGGGAGGATGGGGAAAAGAGCTGAGAGTTTATGCTGAAATGGATTTAT
AGAATATTTGTAAATCTATTTTTAGTGTTTGTTCGTTTTTTTAACTGTTCATTCCTTTGTGCAGAGTGTA
TATCTCTGCCTGGGCAAGAGTGTGGAGGTGCCGAGGTGTCTTCATTCTCTCGCACATTTCCACAGCA
CCTGCTAAGTTTGTATTTAATGGTTTTTGTTTTTGTTTTTGTTTGTTTCTTGAAAATGAGAGAAGAGCC
GGAGAGATGATTTTTATTAATTTTTTTTTTTTTTTTTTTTTTTTACTATTTATAGCTTTAGATAGGGCCTC
CCTTCCCCTCTTCTTTCTTTGTTCTCTTTCATTAAACCCCTTCCCCAGTTTTTTTTTTATACTTTAAACCC
CGCTCCTCATGGCCTTGGCCCTTTCTGAAGCTGCTTCCTCTTATAAAATAGCTTTTGCCGAAACATAG
TTTTTTTTTAGCAGATCCCAAAATATAATGAAGGGGATGGTGGGATATTTGTGTCTGTGTTCTTATAA
TATATTATTATTCTTCCTTGGTTCTAGAAAAATAGATAAATATATTTTTTTCAGGAAATAGTGTGGTGT
TTCCAGTTTGATGTTGCTGGGTGGTTGAGTGAGTGAATTTTCATGTGGCTGGGTGGGTTTTTGCCTTT
TTCTCTTGCCCTGTTCCTGGTGCCTTCTGATGGGGCTGGAATAGTTGAGGTGGATGGTTCTACCCTT
TCTGCCTTCTGTTTGGGACCCAGCTGGTGTTCTTTGGTTTGCTTTCTTCAGGCTCTAGGGCTGTGCTA
TCCAATACAGTAACCACATGCGGCTGTTTAAAGTTAAGCCAATTAAAATCACATAAGATTAAAAATTC
CTTCCTCAGTTGCACTAACCACGTTTCTAGAGGCGTCACTGTATGTAGTTCATGGCTACTGTACTGAC
AGCGAGAGCATGTCCATCTGTTGGACAGCACTATTCTAGAGAACTAAACTGGCTTAACGAGTCACAG
CCTCAGCTGTGCTGGGACGACCCTTGTCTCCCTGGGTAGGGGGGGGGGAATGGGGGAGGGCTGAT
GAGGCCCCAGCTGGGGCCTGTTGTCTGGGACCCTCCCTCTCCTGAGAGGGGAGGCCTGGTGGCTTA
GCCTGGGCAGGTCGTGTCTCCTCCTGACCCCAGTGGCTGCGGTGAGGGGAACCACCCTCCCTTGCT
GCACCAGTGGCCATTAGCTCCCGTCACCACTGCAACCCAGGGTCCCAGCTGGCTGGGTCCTCTTCT
GCCCCCAGTGCCCTTCCCCTTGGGCTGTGTTGGAGTGAGCACCTCCTCTGTAGGCACCTCTCACACT
GTTGTCTGTTACTGATTTTTTTTGATAAAAAGATAATAAAACCTGGTACTTTCTAAA

### SLC4A4

SLC4A4-NRG1 fusion sequence
SEQ ID NO: 384
ACTACCCCATCAACTCCAACTTCAAAGTGGGCTACAACACTCTCTTTTC
CTGTACCTGTGTGCCACCTGACCCAGCTACATCTACATCCACCACTGGG
ACAAGCCATCTTGTAAAATGTGCGGAGAAGGAGAAAACTTTCTGTGTGA
ATG

SEQ ID NO:385=SLC4A4 Exon 1
SEQ ID NO:386=SLC4A4 Exon 2
SEQ ID NO: 387=SLC4A4 Exon 3
SEQ ID NO:388=SLC4A4 Exon 4
SEQ ID NO:389=SLC4A4 Exon 5
SEQ ID NO: 390=SLC4A4 Exon 6
SEQ ID NO:391=SLC4A4 Exon 7
SEQ ID NO:392=SLC4A4 Exon 8
SEQ ID NO:393=SLC4A4 Exon 9
SEQ ID NO:394=SLC4A4 Exon 10
SEQ ID NO:395=SLC4A4 Exon 11
SEQ ID NO:396=SLC4A4 Exon 12
SEQ ID NO:397=SLC4A4 Exon 13
SEQ ID NO: 398=SLC4A4 Exon 14
SEQ ID NO:399=SLC4A4 Exon 15
SEQ ID NO:400=SLC4A4 Exon 16
SEQ ID NO:401=SLC4A4 Exon 17
SEQ ID NO:402=SLC4A4 Exon 18
SEQ ID NO:403=SLC4A4 Exon 19
SEQ ID NO:404=SLC4A4 Exon 20
SEQ ID NO:405=SLC4A4 Exon 21
SEQ ID NO:406=SLC4A4 Exon 22
SEQ ID NO:407=SLC4A4 Exon 23
SEQ ID NO:408=SLC4A4 Exon 24
SEQ ID NO:409=SLC4A4 Exon 25
SEQ ID NO:410=SLC4A4 Exon 26
SEQ ID NO:411=SLC4A4 Exons 1-26
SEQ ID NO:412=SLC4A4 Exons 1-14

SLC4A4 sequence information, providing nucleotide sequence of exons 1-26 below as indicated by alternating underlining, starting with exon 1 underlined, exon 2 not underlined, exon 3 underlined, exon 4 not underlined, and so forth.

SEQ ID NO: 411
GGCGGCGCGCCGGGCAGCGCTTCGGTGGCGGCGGCGGCCGCGGTGGCAGCGAAGGCGGCGGCG
GCGGCGGCAGTGGCAGTGGCCGCTGCAGCCCCACACTCCGCCGCCAAACTGGAGGAGCGACGGAA
GCCAGACCCCAGGAGGATGGAGGATGAAGCTGTCCTGGACAGAGGGGCTTCCTTCCTCAAGCATGT
GTGTGATGAAGAAGAAGTAGAAGGCCACCATACCATTTACATCGGAGTCCATGTGCCGAAGAGTTAC
AGGAGAAGGAGACGTCACAAGAGAAAGACAGGGCACAAAGAAAAGAAGGAAAAGGAGAGAATCTC
TGAGAACTACTCTGACAAATCAGATATTGAAAATGCTGATGAATCCAGCAGCAGCATCCTAAAACCTC
TCATCTCTCCTGCTGCAGAACGCATCCGATTCATCTTGGGAGAGGAGGATGACAGCCCAGCTCCCCC
TCAGCTCTTCACGGAACTGGATGAGCTGCTGGCCGTGGATGGGCAGGAGATGGAGTGGAAGGAAA
CAGCCAGGTGGATCAAGTTTGAAGAAAAAGTGGAACAGGGTGGGGAAAGATGGAGCAAGCCCCAT
GTGGCCACATTGTCCCTTCATAGTTTATTTGAGCTGAGGACATGTATGGAGAAAGGATCCATCATGC
TTGATCGGGAGGCTTCTTCTCTCCCACAGTTGGTGGAGATGATTGTTGACCATCAGATTGAGACAGG
CCTATTGAAACCTGAACTTAAGGATAAGGTGACCTATACTTTGCTCCGGAAGCACCGGCATCAAACC
AAGAAATCCAACCTTCGGTCCCTGGCTGACATTGGGAAGACAGTCTCCAGTGCAAGTAGGATGTTTA
CCAACCCTGATAATGGTAGCCCAGCCATGACCCATAGGAATCTGACTTCCTCCAGTCTGAATGACAT
TTCTGATAAACCGGAGAAGGACCAGCTGAAGAATAAGTTCATGAAAAAATTGCCACGTGATGCAGAA
GCTTCCAACGTGCTTGTTGGGGAGGTTGACTTTTTGGATACTCCTTTCATTGCCTTTGTTAGGCTACA
GCAGGCTGTCATGCTGGGTGCCCTGACTGAAGTTCCTGTGCCCACAAGGTTCTTGTTCATTCTCTTA
GGTCCTAAGGGGAAAGCCAAGTCCTACCACGAGATTGGCAGAGCCATTGCCACCCTGATGTCTGAT
GAGGTGTTCCATGACATTGCTTATAAAGCAAAAGACAGGCACGACCTGATTGCTGGTATTGATGAGT
TCCTAGATGAAGTCATCGTCCTTCCACCTGGGGAATGGGATCCAGCAATTAGGATAGAGCCTCCTAA
GAGTCTTCCATCCTCTGACAAAAGAAAGAATATGTACTCAGGTGGAGAGAATGTTCAGATGAATGGG
GATACGCCCCATGATGGAGGTCACGGAGGAGGAGGACATGGGGATTGTGAAGAATTGCAGCGAAC
TGGACGGTTCTGTGGTGGACTAATTAAAGACATAAAGAGGAAAGCGCCATTTTTTGCCAGTGATTTT
TATGATGCTTTAAATATTCAAGCTCTTTCGGCAATTCTCTTCATTTATCTGGCAACTGTAACTAATGCT
ATCACTTTTGGAGGACTGCTTGGGGATGCCACTGACAACATGCAGGGCGTGTTGGAGAGTTTCCTG
GGCACTGCTGTCTCTGGAGCCATCTTTTGCCTTTTTGCTGGTCAACCACTCACTATTCTGAGCAGCAC
CGGACCTGTCCTAGTTTTTGAGAGGCTTCTATTTAATTTCAGCAAGGACAATAATTTTGACTATTTGG
AGTTTCGCCTTTGGATTGGCCTGTGGTCCGCCTTCCTATGTCTCATTTTGGTAGCCACTGATGCCAGC
TTCTTGGTTCAATACTTCACACGTTTCACGGAGGAGGGCTTTTCCTCTCTGATTAGCTTCATCTTTATC
TATGATGCTTTCAAGAAGATGATCAAGCTTGCAGATTACTACCCCATCAACTCCAACTTCAAAGTGGG
CTACAACACTCTCTTTTCCTGTACCTGTGTGCCACCTGACCCAGCTAATATCTCAATATCTAATGACAC
CACACTGGCCCCAGAGTATTTGCCAACTATGTCTTCTACTGACATGTACCATAATACTACCTTTGACT
GGGCATTTTTGTCGAAGAAGGAGTGTTCAAAATACGGAGGAAACCTCGTCGGGAACAACTGTAATTT
TGTTCCTGATATCACACTCATGTCTTTTATCCTCTTCTTGGGAACCTACACCTCTTCCATGGCTCTGAA
AAAATTCAAAACTAGTCCTTATTTTCCAACCACAGCAAGAAAACTGATCAGTGATTTTGCCATTATCTT
GTCCATTCTCATCTTTTGTGTAATAGATGCCCTAGTAGGCGTGGACACCCCAAAACTAATTGTGCCAA
GTGAGTTCAAGCCAACAAGTCCAAACCGAGGTTGGTTCGTTCCACCGTTTGGAGAAAACCCCTGGTG
GGTGTGCCTTGCTGCTGCTATCCCGGCTTTGTTGGTCACTATACTGATTTTCATGGACCAACAAATTA
CAGCTGTGATTGTAAACAGGAAAGAACATAAACTCAAGAAAGGAGCAGGGTATCACTTGGATCTCTT
TTGGGTGGCCATCCTCATGGTTATATGCTCCCTCATGGCTCTTCCGTGGTATGTAGCTGCTACGGTC
ATCTCCATTGCTCACATCGACAGTTTGAAGATGGAGACAGAGACTTCTGCACCTGGAGAACAACCAA
AGTTTCTAGGAGTGAGGGAACAAAGAGTCACTGGAACCCTTGTGTTTATTCTGACTGGTCTGTCAGT
CTTTATGGCTCCCATCTTGAAGTTTATACCCATGCCTGTACTCTATGGTGTGTTCCTGTATATGGGAG
TAGCATCCCTTAATGGTGTGCAGTTCATGGATCGTCTGAAGCTGCTTCTGATGCCTCTGAAGCATCA
GCCTGACTTCATCTACCTGCGTCATGTTCCTCTGCGCAGAGTCCACCTGTTCACTTTCCTGCAGGTGT
TGTGTCTGGCCCTGCTTTGGATCCTCAAGTCAACGGTGGCTGCTATCATTTTTCCAGTAATGATCTTG
GCACTTGTAGCTGTCAGAAAAGGCATGGACTACCTCTTCTCCCAGCACGACCTCAGCTTCCTGGATG
ATGTCATTCCAGAAAAGGACAAGAAAAAGAAGGAGGATGAGAAGAAAAAGAAAAAGAAGAAGGGAA
GTCTGGACAGTGACAATGATGATTCTGACTGCCCATACTCAGAAAAAGTTCCAAGTATTAAAATTCCA
ATGGACATCATGGAACAGCAACCTTTCCTAAGCGATAGCAAACCTTCTGACAGAGAAAGATCACCAA
CATTCCTTGAACGCCACACATCATGCTGATAAAATTCCTTTCCTTCAGTCACTCGGTATGCCAAGTCC
TCCTAGAACTCCAGTAAAAGTTGTGCCTCAAATTAGAATAGAACTTGAACCTGAAGACAATGATTATT
TCTGGAGGAGCAAGGGAACAGAAACTACATTGTAACCTGTTTGTCTTTCTTAAAACTGACATTTGTTG
TTAATGTCATTTGTTTTTGTTTGGCTGTTTGTTTATTTTTTAACTTTTATTTCGTCTCAGTTTTTGGTCAC
AGGCCAAATAATACAGCGCTCTCTCTGCTTCTCTCTTGCATAGACACAATCAAGACAATAGTGCACCG
TTCCTTAAAAACAGCATCTGAGGAATCCCCCTTTTGTTCTTAAACTTTCAGATGTGTCCTTTGATAACC
AAATTCTGTCACTCAAGACACAGACACGCACAGACCCTGTCCTTTGCCTCTATTAAGCAGAGGATGG
AAGTATTAAGGATTTTGTAACACCTTTTATGAAAATGTTGAAGGAACTTAAAACTTTAGCTTTGGAGC
TGTGCTTACTGGCTTGTCTTTGTCTGGTAGAACAAACCTTGACCTCCAGACAGAGTCCCTTCTCACTT
ATAGAGCTCTCCAGGACTGGAAAAAGTGCTGCTATTTTAACTTGCTCTTGCTTGTAAATCCTAATCTT
AGAGTTATCAAAAGAAGAAAAAACTGAAGGTACTTTACTCCCTATAGAGAAACCATTGCCATCATTGT
AGCAAGTGCTGGAATGTCCCTTTTTTCCTATGCAACTTTTTTTAACCCTTTAATGAACTTATCTGTTGA
GTACATTGAAGAATATTTTTCTTCCTAGATTTTGTTGTTTAAATTATGGGGCCTAACCTGCCACTTATT
TTTTGTCAATTTTTAAAACTTTTTTTTAATTACTGTAAAGAAAATGAATTTTTTCCTGCAGCAGGAAACA
TAGTTTTGAGTAGTTCTACCTCTTATTTGTAGCTGCCAGGCTTTCTGTAAAAATTGTATTGTATATAAT
GTGATTTTTACACATACATACACACACAAATACACAATCTCTAGGGTAAGCCAGAAGGCAAGATCAG
ATTAAAAACACCATGTTTCTAAGCATCCATTTTTCCCTTTCTTTAAAAGAAACTTAACTGTTCTATGAA
GGAGATTGAGGGAGAAGAGACAAACTCCTATGTCATGAGAATAACCGATGTTCTGATAATAGTAGCA
TCTAGGTACAGATGCTGGTTGTATTACCACGTCAATGTCCTATGCAGTATTGTTAGACATTTTCTCATT
TTGAAATATTTGTGTGTTTGTGTATGTGCTCTGTGCCATGGCTGGTGTATATATGTGCAATGTTAGAA
GGCAAAAGAGTGATGGTAGGCAGAGGGCAAAGTCATTGAATCTCTTATGCCAGTTTTCATAAAACCC
AAACCACATATGAAAAAATCCATTAAGGGTCCAAGAAGTCTGTCCATATGAAAATGAGGGTAAATATA
GTTTATTTCCCAGGTATCAGTCATTATAATTGATATAATAGCTCTAACATGCAATATAAAATTCATAGG
AGTATTAATAGCCCATTTACACATCTATAAAATGTAATGGGATTGCAGAGCTGCAGAGTACAGTGTAA
CAGTACTCTCATGCAATTTTTTTCAGGATGCAAAGGCAATTATTCTTTGTAAGCGGGACATTTAGAAT
ATATTTGTGTACATATTATATGTATGTATATTTCAAAGTACCACACTGAAAATTAGACATTTATTAACC
AAATTTAACGTGGTATTTAAAGGTAATATTTTTAATATGATACATTACATATTGTGAATGTATACTAAA
AAAACATTTTAAATGTTAAAATTATAATTTCAGATTCATATAACCACAACTGTGATATATCCTAACTATA
ACCAGTTGTTGAGGGGTATACTAGAAGCAGAATGAAACCACATTTTTTGGTTTGATAATATGCACTTA
TTGACTCCCACTCATTGTTATGTTAATTAAGTTATTATTCTGTCTCCTTGTAATTTTGATTACAAAAATT
TTATTATCCTGAGTTAGCTGTTACTTTTACAGTACCTGATACTCCTAAAACTTTTAACTTATACAAATTA
GTCAATAATGACCCCAATTTTTTCATTAAAATAATAGTGGTGAATTATATGTTATTGTGTTAAAACCTC
ACTTGCCAAATTCTGGCTTCACATTTGTATTTAGGGCTATCCTTAAAATGATGAGTCTATATTATCTAG
CTTTCTATTACCCTAATATAAACTGGTATAAGAAGACTTTCCTTTTTTCTTTATGCATGGAAGCATCAA
TAAATTGTTTAAAAACCATGTATAGTAAATTCAGCTTAACCCGTGATCTTCTTAAGTTAAAGGTACTTT
TGTTTTATAAAAGCTCTAGATAAAACTTTCTTTTCTGATCATGAATCAAGTATCTGTGGTTTCATGCCC
CTCTCTATACCTTTCAAAGAACTCCTGAAGCAACTTAACTCATCATTTCAGCCTCTGAGTAGAGGTAA
AACCTATGTGTACTTCTGTTTATGATCCATATTGATATTTATGACATGAACACAGAATAGTACCTTACA
TTTGCTAAACAGACAGTTAATATCAAATCCTTTCAATATTCTGGGAACCCAGGGAAGTTTTTAAAAAT
GTCATTACTTTCAAAGGAACAGAAGTAGTTAACCAAACTAACAAGCAAAACCTGAGGTTTACCTAGTG
ACACCAAATTATCGGTATTTTAACTGAATTTACCCATTGACTAAGAATGAACCAGATTTGGTGGTGGT
TTTGTTTCTATGCAAACTGGACACAAATTACAACAGTAAATTTTTTTATAAGTGCTTCTCCCTTCTCCA
TGATGTGACTTCCGGAGATAAAGGATTCAAAAGATAAAGACAAAGTACGCTCAGAGTTGTTAACCAG
AAAGTCCTGGCTGTGGTTGCAGAAACACTGTTGGAAGAAAAGAGATGACTAAGTCAAGTGTCTGCCT
TATCAAAAGAGCAAAAATGCCTCTGGTTTTGTGTTTGGGAGAAAAGTATCTTGGACGCACTGTTTTCC
TTGATAAAAGTCATCTTCTCTACTGTGTGAAATGAATACTTGGAATTCTAATTGTTTTGTGTGCCAGG
GGCAGTAATGTCCCTGCCTCTTCTCCCAATCAAGGTTGAGGAGTGGGGCTGGGGAGAGGACTTAAC
TGACTTAAGAAGTAGGAAAACAAAAACCTCTCTCCTCAGCCTTCCACCTCCAAGAGAGGAGGAAAAA
CAGTTGTCTGCTGTCTGTAATTCAGTTTGCGTGTATTTTATGCTCATGCACCAACCCATACAGAGTAA
ATCTTTTATCAACTGTATACTGGTGTTTAATAGAGAATGATTGTCTTCCGAGTTTTTTGGTTCCTTTTTT
AACTGTGTTAAAGTACTTGAAATGTATTGACTGCTGACTATATTTTAAAAACAAAATGAAATAATTTGA
GTTGTATTACAGAGGTTGACATTGTTCAGGGATGGGACAAAGCCTTCTTCAATCCTTTTCATACTACT
TAATGATTTTGGTGCAGGAACCTGAGATTTTCTGATTTATATTTCATGATATTTCACATTTGCTCTTCA
CAGCATGAGCATGAAGCCCAGTGGCACCAAATGGCTGGGTACAATCAAGTGATATTTTGTAGCACCT
CACTATCTGAAAGGCCATGAGTTTTCAGATGATTTCATTGAGCTTCATTGCAGCCTGAAATTTTAAAA
AAGTTGTGTAATACGCCAACCAGTCAAGTTGTGTTTTGGCCAGAGATTTAGATATGTCCAATTTCCTG
GCTCATTTCATTGTGCTCTATGGGTACGTATAAAAAGCAAGAATTCTGTTTCCTAGGCAAACATTGCA
ACTCAGGGCTAAAGTCATCCAGTGAAACTTTTAGAGCCAGAAGTAACTTTGTCCCAGTCCTACAATGT
GAAAAGAGTGAATAGTTGCCTCTTTTTAGCCATTTTCATGGCTGGTACATATTCGTACGCATTACTTTT
CAGAATCAATACGCACTTTCAGATATTCTTATTTTTATTCTCTTAAGTCTTTATTAACTTTGGAGAGAG
AAATGATGCATCTTTTTATTTTAAATGAAGTAGATCAACATGGTGGAACAAAATGATAAAGAACAGAA
AACATTTCAATATATTACTAATAACTTTTTCCAATATAAATCCTAAAATTCCTATAACATAGTATTTTAC
AGTTTTATGAAGCTTTCTATTGTGACTTTTATGGAATTAAGAGATGAAGAAGATGAGATATTTTAGCA
TTTATATTTTTCAAAATTATATGTATACTTAAAAATAAAGTAACTTTATGCA

### ZFAT

ZFAT-NRG1 fusion sequence
SEQ ID NO: 413
ACAGGAAGCACCCTAATGAGGAGTATGCCAACGTGGGCACCGGGGAGCT
GGCAGCGGAGGTGCTCATCCAGCAAGCTACATCTACATCCACCACTGGG
ACAAGCCATCTTGTAAAATGTGCGGAGAAGGAGAAAACTTTCTGTGTGA
ATG

SEQ ID NO: 414=ZFAT Exon 1
SEQ ID NO:415=ZFAT Exon 2
SEQ ID NO:416=ZFAT Exon 3
SEQ ID NO: 417=ZFAT Exon 4
SEQ ID NO: 418=ZFAT Exon 5
SEQ ID NO: 419=ZFAT Exon 6
SEQ ID NO:420=ZFAT Exon 7
SEQ ID NO:421=ZFAT Exon 8
SEQ ID NO:422=ZFAT Exon 9
SEQ ID NO:423=ZFAT Exon 10
SEQ ID NO:424=ZFAT Exon 11
SEQ ID NO: 425=ZFAT Exon 12
SEQ ID NO: 426=ZFAT Exon 13
SEQ ID NO:427=ZFAT Exon 14
SEQ ID NO:428=ZFAT Exon 15
SEQ ID NO:429=ZFAT Exon 16
SEQ ID NO:430=ZFAT Exons 1-16
SEQ ID NO:431=ZFAT Exons 1-12

ZFAT sequence information, providing nucleotide sequence of exons 1-16 below as indicated by alternating underlining, starting with exon 1 underlined, exon 2 not underlined, exon 3 underlined, exon 4 not underlined, and so forth.

SEQ ID NO: 430
ATCCGCCATGTTGGATGCCGCAGATTCGCCATAACCTCGCCGGCTCTTTTCTTAAAAAAATAAAAATA
AAAAGCGAAGCGTCAGCAGGGCGCCCCGCCCCCTCGGTCGGCACGGGAGGGGGCCCGGAAGAGC
CCGAGGCTTTTTTTTCCTCCGCGGTGGGGCGTTGCCATGGAGACGCGGGCGGCAGAAAACACGGCC
ATCTTTATGTGTAAATGTTGTAACCTCTTCTCACCAAATCAGTCGGAACTCCTCTCCCACGTTTCAGA
GAAGCACATGGAAGAAGGGGTTAATGTTGATGAGATTATTATTCCCCTTAGGCCTCTGAGTACACCT
GAACCCCCCAACTCAAGCAAAACCGGAGATGAGTTTTTGGTCATGAAGAGGAAGAGAGGCAGGCCT
AAGGGGTCCACGAAGAAGTCCAGCACAGAAGAGGAGCTGGCAGAAAACATCGTGAGTCCGACTGA
GGACAGCCCGCTGGCTCCGGAGGAAGGGAACAGCCTGCCTCCAAGCAGCTTGGAGTGTAGCAAGT
GCTGTCGGAAGTTCTCCAACACGCGCCAGCTGCGGAAGCACATCTGCATTATCGTGCTGAATTTGGG
TGAGGAGGAAGGAGAAGCAGGTAACGAGTCTGACCTTGAACTAGAAAAGAAGTGTAAGGAAGATGA
TCGGGAAAAAGCCTCGAAAAGACCACGGTCACAGAAAACAGAGAAAGTCCAGAAGATCTCAGGAAA
GGAGGCCAGACAGCTTTCTGGGGCGAAGAAACCCATCATAAGTGTGGTTTTAACTGCACACGAAGC
AATTCCAGGTGCTACCAAGATTGTGCCAGTGGAGGCTGGGCCCCCTGAAACAGGAGCTACAAATTCT
GAGACCACTTCAGCAGACCTGGTGCCTCGGAGAGGCTACCAGGAATACGCCATTCAGCAGACACCT
TATGAGCAACCAATGAAGTCAAGCAGGCTAGGTCCCACTCAGCTCAAAATCTTCACTTGTGAATACT
GCAACAAGGTCTTCAAGTTCAAGCACTCGCTGCAGGCCCACCTGAGGATCCACACCAATGAAAAGCC
ATACAAGTGCCCCCAGTGCAGCTATGCCAGTGCCATCAAGGCCAACCTCAATGTGCACCTGCGCAA
GCACACTGGAGAGAAGTTCGCCTGCGACTATTGCTCGTTCACCTGCCTGAGCAAGGGCCACCTCAA
GGTGCACATCGAGCGAGTGCACAAGAAGATCAAGCAGCACTGCCGCTTCTGCAAGAAGAAGTACTC
TGACGTCAAGAACCTCATCAAGCACATCCGAGACGCGCATGACCCACAGGACAAGAAGGTCAAAGA
GGCCTTGGACGAGCTCTGCCTGATGACGAGGGAGGGCAAGCGGCAGCTGCTCTATGACTGCCACAT
CTGTGAGCGCAAGTTCAAGAACGAGCTGGACCGTGACCGCCATATGCTGGTCCACGGAGACAAGTG
GCCTTTTGCCTGTGAGCTCTGTGGCCATGGGGCCACCAAGTACCAGGCGCTGGAACTGCATGTCAG
GAAGCACCCCTTCGTGTACGTCTGTGCCGTCTGCCGCAAGAAGTTCGTCAGCTCCATCAGGCTGCG
CACCCACATCAAAGAGGTGCACGGGGCTGCCCAGGAGGCCTTGGTCTTCACCAGTTCCATCAACCA
GAGCTTCTGCCTCCTGGAACCTGGTGGGGACATCCAGCAAGAAGCTCTGGGGGACCAGCTACAGCT
GGTGGAAGAGGAGTTTGCCCTCCAGGGCGTGAATGCACTCAAGGAAGAGGCCTGTCCTGGGGACA
CTCAGCTGGAGGAGGGCCGGAAGGAGCCGGAGGCCCCTGGGGAAATGCCTGCCCCAGCTGTGCAC
CTGGCCTCCCCGCAGGCCGAAAGCACAGCCCTGCCACCCTGTGAGCTGGAAACCACCGTGGTCTCC
TCCTCAGACCTGCATTCTCAAGAGGTGGTTTCAGATGATTTTTTGTTGAAAAATGATACCTCCTCCGC
AGAGGCTCATGCTGCTCCTGAGAAGCCCCCAGACATGCAGCACAGAAGCTCAGTCCAGACGCAAGG
TGAAGTGATCACACTACTGCTGTCCAAGGCCCAGAGTGCTGGGTCAGATCAGGAAAGCCATGGCGC
CCAGAGCCCCCTAGGGGAAGGGCAGAACATGGCTGTGCTTTCAGCTGGTGACCCAGATCCCAGCAG
GTGTCTCAGGTCAAACCCAGCTGAGGCCTCAGACCTCCTCCCTCCAGTAGCTGGTGGTGGGGACAC
CATCACACATCAGCCTGACTCTTGCAAAGCTGCCCCTGAGCACCGGTCAGGCATCACCGCTTTCATG
AAGGTCCTGAACAGTTTACAGAAGAAGCAAATGAACACCAGCTTGTGTGAGCGGATCCGGAAGGTT
TATGGAGACCTGGAGTGTGAATACTGTGGCAAACTTTTTTGGTACCAAGTGCATTTTGATATGCATGT
CCGCACCCACACCCGGGAACATCTGTATTATTGCTCTCAGTGTCATTATTCTTCCATCACCAAAAACT
GCCTTAAACGCCACGTAATTCAGAAACACAGTAACATCTTGCTGAAGTGTCCCACCGATGGCTGTGA
CTACTCAACTCCAGATAAATATAAGCTACAGGCACATCTTAAAGTTCACACAGCACTGGACAAAAGG
AGTTATTCTTGTCCTGTTTGTGAAAAGTCTTTTTCAGAGGATCGATTGATAAAGTCACATATCAAGAC
CAACCATCCTGAGGTCTCCATGAGCACCATTTCTGAGGTTCTCGGGAGGAGGGTTCAGCTGAAAGG
GCTAATTGGAAAGAGAGCCATGAAATGCCCATATTGTGACTTTTATTTCATGAAGAATGGCTCAGACC
TTCAGCGTCATATTTGGGCTCATGAAGGTGTGAAGCCCTTCAAGTGTTCTTTGTGTGAGTATGCAACT
CGTAGCAAGAGTAACCTCAAGGCTCATATGAATCGTCACAGCACTGAGAAAACCCACCTATGTGACA
TGTGTGGCAAGAAATTCAAATCAAAAGGGACACTGAAAAGTCACAAACTCCTTCACACTGCAGATGG
GAAGCAGTTTAAGTGCACGGTGTGTGACTACACAGCGGCCCAGAAGCCACAGCTGCTGCGGCACAT
GGAACAGCATGTCTCCTTCAAGCCTTTCCGCTGTGCCCATTGCCATTACTCCTGCAACATATCTGGCT
CTCTGAAGCGGCACTACAACAGGAAGCACCCTAATGAGGAGTATGCCAACGTGGGCACCGGGGAGC
TGGCAGCGGAGGTGCTCATCCAGCAAGGTGGTTTGAAGTGTCCTGTTTGCAGCTTTGTATATGGCAC
CAAATGGGAGTTCAATAGGCACTTGAAGAACAAACATGGCTTGAAGGTGGTGGAAATTGATGGAGA
CCCCAAGTGGGAGACAGCAACAGAAGCTCCTGAGGAGCCCTCCACCCAGTATCTCCACATCACAGA
GGCCGAAGAAGACGTTCAAGGGACACAGGCAGCGGTGGCCGCGCTCCAGGACCTGAGATACACCT
CTGAGAGTGGCGACCGACTGGACCCCACGGCCGTGAACATCCTGCAGCAGATCATTGAGCTGGGCG
CCGAGACCCATGACGCCACTGCCCTTGCCTCGGTGGTTGCCATGGCACCAGGGACGGTGACTGTGG
TTAAGCAGGTCACCGAGGAGGAGCCCAGCTCCAACCACACGGTCATGATCCAGGAGACGGTCCAGC
AAGCGTCCGTGGAGCTTGCCGAGCAGCACCACCTGGTGGTGTCCTCCGACGACGTGGAGGGCATT
GAGACGGTGACTGTCTACACGCAGGGGGGGGAGGCCTCGGAGTTCATCGTCTACGTGCAGGAGGC
CATGCAGCCTGTGGAGGAGCAGGCTGTGGAGCAGCCGGCCCAGGAACTCTAGAGGACATGTGGCA
TCGGATGGCCACAGGGGGGGGCTGCCAGGCTCTGCAGGCACCCAGGGTGGGGAGGCCACCCTTCC
TGCCCTACCCGCAGAATGGTGCTCTCCTTTGCCCTCCCTGCCCAGCAGCCTGATAGGACTCTCCTAG
TCCAACTTGGGGTGGGCAAGGCAGTCAGCATCACCAGCAATACCACAGGACCCTCACCCCAGCATA
GACACACACCCCCTGACCCTTACCATCTGCTTCCTGAAAGACTTCAGTGTCAGCTCCCCTACACACAC
CCCACACCTTCACCCCTTGCTTCAAGATTCAAACAGAGACTCCCAGTCCCCCTCAGCATCTTCCCTGA
ATCACAGCCCCAGCTCCTTGACCCCCATCTAGGTGCCAAATGTTCATCTGCAACCGCTATGCAGTCT
GGTGAGAGGGAGACAGCCATCACATAGAAAGTGACCGTACGGGTTTTTAATCACTGCTGGGTGGGG
TGGGGGTAGGGGGATTGTCCTGGCTTTGTCGACAAAGTCCCACTTCCCCGAGTATTAAGGGCCCTT
GGTATCAAGTGAGGTAAATTCACCCATCACAGGGTCTCGCCCTACCATCCTGGAATTATTTCACTTTT
AAGATAAATGCACTATTTCACTGTTCGCCTCCCATTCTAAGGAGGTGAGGTGGTTGGAATAAAAACA
GTTCCTGTCTGAA

### DSCAML1

DSCAML1-NRG1 fusion sequence
SEQ ID NO: 432
GCCTCATCCCCTCTTCAGTGCAGGAATATGTTAGCGTTGTATCTTGGGA
GAAAGACACAGTCTCCATCATCCCAGCCTTGCCTCCCCGATTGAAAGAG
ATGAAAAGCCAGGAATCGGCTGCAGGTTCCAAACTAGTCCTTCGGTGTG
AAA

SEQ ID NO:433=DSCAML1 Exon 1
SEQ ID NO: 434=DSCAML1 Exon 2
SEQ ID NO: 435=DSCAML1 Exon 3
SEQ ID NO: 436=DSCAML1 Exon 4
SEQ ID NO: 437=DSCAML1 Exon 5
SEQ ID NO: 438=DSCAML1 Exon 6
SEQ ID NO: 439=DSCAML1 Exon 7
SEQ ID NO: 440=DSCAML1 Exon 8
SEQ ID NO:441=DSCAML1 Exon 9
SEQ ID NO:442=DSCAML1 Exon 10
SEQ ID NO:443=DSCAML1 Exon 11
SEQ ID NO: 444=DSCAML1 Exon 12
SEQ ID NO: 445=DSCAML1 Exon 13
SEQ ID NO: 446=DSCAML1 Exon 14
SEQ ID NO: 447=DSCAML1 Exon 15
SEQ ID NO: 448=DSCAML1 Exon 16
SEQ ID NO: 449=DSCAML1 Exon 17
SEQ ID NO:450=DSCAML1 Exon 18
SEQ ID NO: 451=DSCAML1 Exon 19
SEQ ID NO: 452=DSCAML1 Exon 20
SEQ ID NO: 453=DSCAML1 Exon 21
SEQ ID NO: 454=DSCAML1 Exon 22
SEQ ID NO: 455=DSCAML1 Exon 23
SEQ ID NO: 456=DSCAML1 Exon 24
SEQ ID NO:457=DSCAML1 Exon 25
SEQ ID NO: 458=DSCAML1 Exon 26
SEQ ID NO:459=DSCAML1 Exon 27
SEQ ID NO: 460=DSCAML1 Exon 28
SEQ ID NO: 461=DSCAML1 Exon 29
SEQ ID NO:462=DSCAML1 Exon 30
SEQ ID NO: 463=DSCAML1 Exon 31
SEQ ID NO: 464=DSCAML1 Exon 32
SEQ ID NO: 465=DSCAML1 Exon 33
SEQ ID NO:466=DSCAML1 Exons 1-33
SEQ ID NO: 467=DSCAML1 Exon 1-3

DSCALM1 sequence information, providing nucleotide sequence of exons 1-33 below as indicated by alternating underlining, starting with exon 1 underlined, exon 2 not underlined, exon 3 underlined, exon 4 not underlined, and so forth.

SEQ ID NO: 466
AGCCGAGCGCTGGGCTGAGGAGCAGAGAGAGCGGGGCGCCGAGTGCGGGCGGCTGGGAGCGCG
CTGAGCGGGGGAGAGGCGCTGCCGCACGGCCGGCCACAGGACCACCTCCCCGGAGAATAGGGCCT
CTTTATGGCATGTGGCTGGTAACTTTCCTCCTGCTCCTGGACTCTTTACACAAAGCCCGCCCTGAAGA
TGTTGGCACCAGCCTCTACTTTGTAAATGACTCCTTGCAGCAGGTGACCTTTTCCAGCTCCGTGGGG
GTGGTGGTGCCCTGCCCGGCCGCGGGCTCCCCCAGCGCGGCCCTTCGATGGTACCTGGCCACAGG
GGACGACATCTACGACGTGCCGCACATCCGGCACGTCCACGCCAACGGGACGCTGCAGCTCTACCC
CTTCTCCCCCTCCGCCTTCAATAGCTTTATCCACGACAATGACTACTTCTGCACCGCGGAGAACGCTG
CCGGCAAGATCCGGAGCCCCAACATCCGCGTCAAAGCAGTTTTCAGGGAACCCTACACCGTCCGGG
TGGAGGATCAAAGGTCAATGCGTGGCAACGTGGCCGTCTTCAAGTGCCTCATCCCCTCTTCAGTGCA
GGAATATGTTAGCGTTGTATCTTGGGAGAAAGACACAGTCTCCATCATCCCAGAACACAGGTTTTTTA
TTACCTACCACGGCGGGCTGTACATCTCTGACGTACAGAAGGAGGACGCCCTCTCCACCTATCGCTG
CATCACCAAGCACAAGTATAGCGGGGAGACCCGGCAGAGCAATGGGGCACGCCTCTCTGTGACAGA
CCCTGCTGAGTCGATCCCCACCATCCTGGATGGCTTCCACTCCCAGGAAGTGTGGGCCGGCCACAC
CGTGGAGCTGCCCTGCACCGCCTCGGGCTACCCTATCCCCGCCATCCGCTGGCTCAAGGATGGCCG
GCCCCTCCCGGCTGACAGCCGCTGGACCAAGCGCATCACAGGGCTGACCATCAGCGACTTGCGGAC
CGAGGACAGCGGCACCTACATTTGTGAGGTCACCAACACCTTCGGTTCGGCAGAGGCCACAGGCAT
CCTCATGGTCATTGATCCCCTTCATGTGACCCTGACACCAAAGAAGCTGAAGACCGGCATTGGCAGC
ACGGTCATCCTCTCCTGTGCCCTGACGGGCTCCCCAGAGTTCACCATCCGCTGGTATCGCAACACGG
AGCTGGTGCTGCCTGACGAGGCCATCTCCATCCGCGGGCTCAGCAACGAGACGCTGCTCATCACCT
CGGCCCAGAAGAGCCATTCCGGGGCCTACCAGTGCTTCGCTACCCGCAAGGCCCAGACCGCCCAG
GACTTTGCCATCATTGCACTTGAGGATGGCACGCCCCGCATCGTCTCGTCCTTCAGCGAGAAGGTGG
TCAACCCCGGGGAGCAGTTCTCACTGATGTGTGCGGCCAAGGGCGCCCCGCCCCCCACGGTCACCT
GGGCCCTCGACGATGAGCCCATCGTGCGGGATGGCAGCCACCGCACCAACCAGTACACCATGTCGG
ACGGCACCACCATCAGCCACATGAACGTCACAGGCCCCCAGATCCGCGACGGGGGCGTGTACCGGT
GCACAGCGCGGAACTTGGTGGGCAGTGCTGAATATCAGGCGCGAATAAACGTAAGAGGCCCACCCA
GCATCCGGGCTATGCGGAACATCACAGCAGTCGCCGGGGGGACACCCTTATCAACTGCAGGGTCA
TCGGCTATCCCTACTACTCCATCAAGTGGTACAAGGATGCCCTGCTGCTGCCAGACAACCACCGCCA
GGTGGTGTTTGAGAATGGGACCCTCAAGCTGACTGACGTGCAGAAGGGCATGGATGAGGGGGAGT
ACCTGTGCAGTGTCCTCATCCAGCCCCAGCTCTCCATCAGCCAGAGCGTTCACGTAGCCGTCAAAGT
GCCCCCTCTGATCCAGCCCTTCGAATTCCCACCCGCCTCCATCGGCCAGCTGCTCTACATTCCCTGT
GTGGTGTCCTCGGGGGACATGCCCATCCGTATCACCTGGAGGAAGGACGGACAGGTGATCATCTCA
GGCTCGGGCGTGACCATCGAGAGCAAGGAATTCATGAGCTCCCTGCAGATCTCTAGCGTCTCCCTC
AAGCACAACGGCAACTATACATGCATCGCCAGCAACGCAGCCGCCACCGTGAGCCGGGAGCGCCA
GCTCATCGTGCGTGTGCCCCCTCGATTTGTGGTGCAACCCAACAACCAGGATGGCATCTACGGCAAA
GCTGGTGTGCTCAACTGCTCGGTGGACGGCTACCCCCCACCCAAGGTCATGTGGAAGCATGCCAAG
GGGAGCGGGAACCCCCAGCAGTACCACCCTGTGCCCCTCACTGGCCGCATCCAGATCCTGCCCAAC
AGCTCGCTGCTGATCCGCCACGTCCTAGAAGAGGACATCGGCTACTACCTCTGCCAGGCCAGCAAC
GGCGTAGGCACCGACATCAGCAAGTCCATGTTCCTCACAGTCAAGATCCCGGCCATGATCACTTCCC
ACCCCAACACCACCATCGCCATCAAGGGCCATGCGAAGGAGCTAAACTGCACGGCACGGGGTGAGC
GGCCCATCATCATCCGCTGGGAGAAGGGGGACACAGTCATCGACCCTGACCGCGTCATGCGGTATG
CCATCGCCACCAAGGACAACGGCGACGAGGTCGTCTCCACACTGAAGCTCAAGCCCGCTGACCGTG
GGGACTCTGTGTTCTTCAGCTGCCATGCCATCAACTCGTATGGGGAGGACCGGGGCTTGATCCAACT
CACTGTGCAAGAGCCCCCCGACCCCCCAGAGCTGGAGATCCGGGAGGTGAAGGCCCGGAGCATGA
ACCTGCGCTGGACCCAGCGATTCGACGGGAACAGCATCATCACGGGCTTCGACATTGAATACAAGA
ACAAATCAGATTCCTGGGACTTCAAGCAGTCCACACGCAACATCTCCCCCACCATCAACCAGGCCAA
CATTGTGGACTTGCACCCGGCATCTGTGTACAGCATCCGCATGTACTCTTTCAACAAGATTGGCCGC
AGTGAACCAAGCAAGGAGCTCACCATCAGCACTGAGGAGGCCGCTCCCGATGGGCCCCCCATGGAT
GTTACCTTGCAGCCAGTGACCTCACAGAGCATCCAGGTGACCTGGAAGGCACCCAAGAAGGAGCTG
CAGAACGGTGTCATCCGGGGCTACCAGATTGGCTACAGAGAGAACAGCCCCGGCAGCAACGGGCA
GTACAGCATCGTGGAGATGAAGGCCACGGGGGACAGCGAGGTCTACACCCTGGACAACCTCAAGAA
GTTCGCCCAGTATGGGGTGGTGGTCCAAGCCTTCAATCGGGCTGGCACGGGGCCCTCTTCCAGCGA
GATCAATGCCACCACTCTGGAGGATGTGCCCAGCCAGCCCCCTGAGAACGTCCGGGCCCTGTCCAT
CACTTCTGACGTGGCCGTCATCTCCTGGTCAGAGCCCCCGCGCAGCACCCTCAATGGCGTCCTCAAA
GGCTATCGGGTCATCTTCTGGTCCCTCTATGTTGATGGGGAGTGGGGCGAGATGCAGAACATCACC
ACCACGCGGGAGCGGGTGGAGCTGCGGGGCATGGAGAAGTTCACCAACTACAGCGTCCAGGTGCT
GGCCTACACCCAGGCTGGGGACGGCGTACGCAGCAGTGTGCTCTACATCCAGACCAAGGAGGACG
TTCCAGGTCCCCCTGCTGGCATCAAAGCTGTCCCTTCATCAGCTAGCAGTGTGGTTGTGTCTTGGCT
CCCCCCTACCAAGCCCAACGGGGTGATCCGCAAGTACACCATCTTCTGTTCCAGCCCCGGGTCTGG
CCAGCCGGCTCCCAGCGAGTACGAGACGAGTCCAGAGCAGCTCTTCTACCGGATCGCCCACCTAAA
CCGCGGTCAGCAGTATCTGCTGTGGGTGGCCGCCGTCACCTCTGCCGGCCGGGGCAACAGCAGCG
AGAAGGTGACCATCGAGCCTGCTGGCAAGGCCCCAGCAAAGATCATCTCCTTTGGGGGCACCGTGA
CAACACCTTGGATGAAAGATGTTCGGCTGCCTTGCAATTCAGTGGGAGATCCAGCCCCTGCTGTGAA
GTGGACCAAGGACAGTGAAGACTCGGCCATTCCAGTGTCCATGGATGGGCACCGGCTCATCCACAC
CAATGGCACACTGCTGCTGCGTGCAGTGAAGGCTGAGGACTCTGGCTACTACACGTGCACGGCCAC
CAACACTGGTGGCTTTGACACCATCATCGTCAACCTTCTGGTGCAAGTTCCCCCGGACCAGCCCCGC
CTCACTGTCTCCAAAACCTCAGCTTCGTCCATCACCCTGACCTGGATTCCAGGTGACAATGGGGGCA
GCTCCATCCGAGGCTTCGTGCTACAGTACTCGGTGGACAACAGCGAGGAGTGGAAGGATGTGTTCA
TCAGCTCCAGCGAGCGCTCCTTCAAGCTGGACAGCCTCAAGTGTGGCACGTGGTACAAGGTGAAGC
TGGCAGCCAAGAACAGCGTGGGCTCTGGGCGCATCAGCGAGATCATCGAGGCCAAGACCCACGGG
CGGGAGCCCTCCTTCAGCAAAGACCAACACCTCTTCACCCACATCAACTCCACGCATGCTCGGCTTA
ACCTGCAGGGCTGGAACAATGGGGGCTGCCCTATCACAGCCATCGTTCTGGAGTACCGGCCCAAGG
GGACCTGGGCCTGGCAGGGCCTCCGGGCCAACAGCTCCGGGGAGGTGTTTCTGACGGAACTGCGA
GAGGCCACGTGGTACGAGCTGCGCATGAGGGCTTGCAACAGTGCGGGCTGCGGCAATGAAACAGC
CCAGTTCGCCACCCTGGACTACGATGGCAGCACCATTCCACCCATCAAGTCTGCTCAAGGTGAAGG
GGATGATGTGAAGAAGCTGTTCACCATCGGCTGCCCTGTCATCCTGGCCACACTGGGGGTGGCACT
GCTCTTCATCGTACGCAAGAAGAGGAAGGAGAAACGGCTGAAGCGACTCCGAGATGCAAAGAGTTT
GGCAGAAATGTTGATAAGCAAGAACAATAGAAGCTTTGACACCCCTGTGAAAGGGCCACCCCAGGG
CCCACGGCTACACATTGACATCCCCAGGGTCCAGCTGCTCATCGAGGACAAAGAAGGCATCAAGCA
ACTGGGAGATGACAAGGCCACCATCCCTGTGACAGATGCTGAGTTCAGCCAAGCTGTCAACCCACA
GAGCTTCTGTACTGGCGTCTCCTTGCACCACCCAACCCTCATCCAGAGCACAGGACCCCTCATCGAC
ATGTCTGACATCCGGCCAGGAACCAATCCAGTGTCCAGGAAGAATGTGAAGTCAGCCCACAGCACC
CGGAACCGGTACTCAAGCCAGTGGACCCTGACCAAGTGCCAGGCCTCCACACCTGCCCGCACCCTC
ACCTCCGACTGGCGCACCGTGGGCTCCCAGCATGGTGTCACGGTCACTGAGAGTGACAGCTACAGT
GCCAGCCTGTCCCAGGACACAGACAAAGGAAGGAACAGCATGGTGTCCACTGAGAGTGCCTCTTCC
ACCTACGAGGAGCTGGCCCGGGCCTATGAGCATGCCAAGCTGGAGGAGCAGCTGCAGCACGCCAA
GTTTGAGATCACCGAGTGCTTCATCTCTGACAGTTCCTCTGACCAGATGACCACAGGCACCAACGAG
AACGCCGACAGCATGACATCCATGAGCACACCCTCAGAGCCTGGCATCTGCCGCTTTACCGCCTCAC
CACCCAAGCCCCAGGATGCGGACCGGGGCAAAAACGTGGCTGTGCCCATCCCTCACCGGGCCAACA
AGAGTGACTACTGCAACCTGCCCCTGTATGCCAAGTCAGAGGCCTTCTTTCGAAAGGCAGATGGAC
GTGAGCCCTGCCCCGTGGTCCCACCCCGTGAGGCCTCCATCCGGAACCTGGCTCGAACCTACCACA
CCCAGGCTCGCCACCTGACCCTGGACCCTGCCAGCAAGTCCTTGGGCCTTCCCCACCCAGGGGCCC
CCGCTGCCGCCTCCACAGCCACCTTACCTCAGAGGACTCTGGCCATGCCAGCCCCCCCAGCCGGCA
CAGCCCCCCCAGCCCCCGGCCCCACCCCTGCTGAGCCACCCACCGCCCCCAGCGCTGCCCCTCCGG
CCCCCAGCACCGAGCCTCCACGAGCCGGGGGCCCACACACCAAAATGGGGGGCTCCAGGGACTCG
CTTCTCGAGATGAGCACATCGGGGGTAGGGAGGTCTCAGAAGCAGGGGGCCGGGGCCTACTCCAA
ATCCTACACCCTGGTGTAGGGCCCGCAGGAAGAGCAGCCACGCCTGGACCGCGCCGCGCCGCAGC
CCCACACGCCAGCTCGGCTGTTTTTCTGCATTATTTATATTCAACTGACAGACAAAAACCAACCAACG
ACAAAACAAAAACCCCCAATCATGAACGCCTGTACATAGAACTCTTTTGTACAAATGAAACTATTTTCT
TCTTCTCCATGAAGCCAGGGCACAAAGAATTTGACAGTACAAGTCAAATCCCCCACCCCACAAAATAT
GTGTGGAGATATATATACATATATAGACAGACAGGAACGCGTCCACGAGCTATATATCTATATATTTC
TCTCACCCTATTTTGAGACAGAGGCACAAAGACTCAGCAATTTTTTTCCCTCCTCCTCACCTTCCCCC
CAGTCTAGGTGGTTTTGACAAAGACCAAAATCCCAACTCAGAGACACTGCATGCGATTTTACTGTTCC
AAGAAAACCAGGAGTTGCTTCAATTTGCAGATGCTTATGTGTTAATACCTTTTTCTATGAAAAAAGAC
CCAGCGCCGTGTGCAATAAAGGTTATGTTTCTA

### Further SDC4-NRG1 Fusion Sequence

SEQ ID NO: 468
ATGTGTCCAACAAGGTGTCAATGTCCAGCACTGTGCAGGGCAGCAACAT
CTTTGAGAGAACGGAGGTCCTGGCAGCCTTGCCTCCCCGATTGAAAGAG
ATGAAAAGCCAGGAATCGGCTGCAGGTTCCAAACTAGTCCTTCGGTGTG
AAA

### Further CD44-NRG1 Fusion Sequence

SEQ ID NO: 469
TTTCTACTGTACACCCCATCCCAGACGAAGACAGTCCCTGGATCACCGA
CAGCACAGACAGAATCCCTGCTACCACTACATCTACATCCACCACTGGG
ACAAGCCATCTTGTAAAATGTGCGGAGAAGGAGAAAACTTTCTGTGTGA
ATG

### RBPMS

RBPMS (NM_006867.4) sequence information, providing nucleotide sequence of exons 1-10 below as indicated by alternating underlining, starting with exon 1 underlined, exon 2 not underlined, exon 3 underlined, exon 4 not underlined, and so forth.
SEQ ID NO:471=RBPMS exon 1
SEQ ID NO:472=RBPMS exon 2
SEQ ID NO:473=RBPMS exon 3
SEQ ID NO:474=RBPMS exon 4
SEQ ID NO:475=RBPMS exon 5
SEQ ID NO:476=RBPMS exon 6
SEQ ID NO:477=RBPMS exon 7
SEQ ID NO: 478=RBPMS exon 8
SEQ ID NO: 479=RBPMS exon 9
SEQ ID NO:480=RBPMS exon 10
SEQ ID NO:481=RBPMS exon 1-10
SEQ ID NO: 482=RBPMS exon 1-5

SEQ ID NO: 481
AGAGCCTGCCTGGAGCGCGTACTCAGCGGCTCTCGGGTCCCAGCGTCCCAGCCGCGGCCCGCGCT
CCTCCGCCCCGCTCCTCCTCCTCCTCTTCCTCCTCCTCCTCCTCTCTAGGCACCCCCGTCCCCTCCTT
CCAGCGGCTGCAGCCCCCAGCCCCAACTCTCCGCGCTTACTCCTGGGACGCGCGTCCTCGCCCCAT
CCTTTGCTTCCTTCCTTCCTTCCTTCTTCCTTCCTCCCCTGGCTCCCGCCCTCCCTCTCCAGGTCGCCC
TCCCGGGGCCCGATTGTCTCGGTGCCCCGCTCCCGGCCCGCGCCCTGCCCCGTCTCTCCCTTGCAC
TTCCTGAGTCGCCCGCCGCCGCCGTCGCAGACTCGCCGCGGGAGCCCCAGCCCAACCCGAGCCCG
ACAGCCACTGCCCCGGCTCCAGCTCCAGCCCCACAGCCCGCGGCGCCCGCCCGAGGGAGCCCCGG
CGCCCGGGGAAGGCTCCAGTGGGCTAGCGCGCCCTCGCCCAGCCCCGCGCCCCAGCCCTGCCCGG
CCCGGCGAGGAAGGACCGGGAAGATGAACAACGGCGGCAAAGCCGAGAAGGAGAACACCCCGAG
CGAGGCCAACCTTCAGGAGGAGGAGGTCCGGACCCTATTTGTCAGTGGCCTTCCTCTGGATATCAA
ACCTCGGGAGCTCTATCTGCTTTTCAGACCATTTAAGGGCTATGAGGGTTCTCTTATAAAGCTCACAT
CTAAACAGCCTGTAGGTTTTGTCAGTTTTGACAGTCGCTCAGAAGCAGAGGCTGCAAAGAATGCTTT
GAATGGCATCCGCTTCGATCCTGAAATTCCGCAAACACTACGACTAGAGTTTGCTAAGGCAAACACG
AAGATGGCCAAGAACAAACTCGTAGGGACTCCAAACCCCAGTACTCCTCTGCCCAACACTGTACCTC
AGTTCATTGCCAGAGAGCCATATGAGCTCACAGTGCCTGCACTTTACCCCAGTAGCCCTGAAGTGTG
GGCCCCGTACCCTCTGTACCCAGCGGAGTTAGCGCCTGCTCTACCTCCTCCTGCTTTCACCTATCCC
GCTTCACTGCATGCCCAGATGCGCTGGCTCCCTCCCTCCGAGGCTACTTCTCAGGGCTGGAAGTCCC
GTCAGTTCTGCTGAATACTATACCCTTCAGCAATGGCTACTAGAAGGACGAACAATTGCCCTCCTTTG
GAAGTACGGCTAATAGAAGCCCTAGATCCGAATAAGATCCGAATAAGAATATGTAATGGACCAGGCG
CAGTGCCTCACGCCTGTCATCCCAGCACTTTGGGAGGCTGAGGCAGGCGGATCACTTGATGCCAGA
AGTTTGAGACCAGCCCAGCCAACATGGTCCCAGGTGTGTGATGGCGGCTGCAATCTGTCTTGTGGG
TATTAATGCAATCTTCAGTGGTGGCTACTGTTCTCTAGCTGTTCTACAAAACTGGAGCATGCTGGCTT
GAAAAACCCTTGCCCAGTTTTGATCCCTTCAAGACTTTGTCACAGCCTCTATCACACATCTGTTTTTCT
CGAAGAAAAAAATATAATTAATAAAAATGTTTTACTCTTTTACACTGTATAATTGTAAGAAATAGCGTA
TTATTTGTGAATGCATGGTCTGAAATTTCTGTACAGTTTGGAGATATTTTCAAACGAAACGTAAGAGA
AGTCAACAATAAAACCAAGAAAAGTGAGTATTTTTATACCAAACATTTTAAGTATGCTGGGATGGACG
ATCTTACACTGGTTTGGATCACAGTTTTTGTTCTTGACTTTTGAATGCTTGTAATTAAAAATATCTATTT
TTTTCCTCTGAAGTAAGTTGCATGTTTGAGGCATGTTTCCAAATATTATCAAAATATCCTGAATTGTAT
TGTGAATATATAGAAATCTGTGCCTGGCCGGAGTCCAGGGTAAATTAGTAGCATGGTGTTAGATGTT
AGAAACAGAACTGTTATTTGCAGTGTTAGGTCTAGGATCCCAGTTCTAGTAGGACAGCCCTGCAAGA
CAATCAACCAGAAGCCTCCAGGAGCTTCTACCTATGGCTTATTCACAACTGGGCAAGAAAACATCATT
GGTAAGAACTGCTGAGTGTGCCCTTAGAAAGCCCTAGTAGCTCCAGCTGTGACTATATCAACTGTGT
GCCAAGTGTGACTTTGTACAGTTTTATGTTTCCACTCTCCTGTATGTGTAGCCACTCGATGCCTAACC
TACCTTCCACAAGCCAGCCCCGCATCCCTGCTCCCGCAGTGTAAGTGCAGAGCCTGCCTCACTGGTA
AGGGAAAACCTTGGCTTGGGAGGCCAGCCCTGGCCCTTGAAGGGGTTGGCTGTGCCCAGCCCACCT
GGCTGCAGTGGGCAGCTCATGTCTGTATCTCCAAAGTGATGTTTGTTTGCAAAACACCGGCTGAACT
GAGCTGGTGTTGCCAACTCTTGGCAGCACTGGGCCAAACCGACCACATACCATGAGCTCCCAAATG
GCGTGTGCTCACTGTGAGACGTCCTGCCACACCCCACAGGAGACGGAGGCAGTGGGCATTTGGAAC
CAATTCTATTCAGACTTCGTCAAAGCCAAAGTCAGTCTGGTGTTGTCAGTTGACACATCTCCAGAGTT
CATGACAGCTCAACCTCTCCCCTTGTACAGAAGCCATTTTTGTAAAACCACACTGACCTAAATTCAGC
TGCCAAACACAGTCTTTCCTATTGATCCGCTCGGCTTATGTTGAAAATTTCAATGTCATGATTACCTG
GTTGGTTTGGGTTTTTGTTTTGTTATTTTCCATTAGAAATATAAAGATGTCAAGAAGCTTTTAAAGGTC
AACACAAAAAACCAAGGCCAGGAGTGAGGGGCTCTTTCTTACCGTAAATAAGGGGAAAAGGCAGTT
AGCTCAAGGACTTGTGACGGATCCACTTTGGTGTTCAAGGACCTGCTTATGCCCTCAGTGCCAATCG
GCTCTTGGTGAGATGACTGTACTCCTAAGGAAAATAGCCACTTCTGCAGTCTATTATGCTTTTATAAC
TGTTTAAAGGTACTTTTCTATTGTCATTTTTAAAAAATAAAGTGCTTATTCCAGCTGTCA

### ATP1B1

ATP1B1 sequence information, providing nucleotide sequence of exons 1-6 below as indicated by alternating underlining, starting with exon 1 underlined, exon 2 not underlined, exon 3 underlined, exon 4 not underlined, and so forth.

| | |
|---|---|
| SEQ ID NO: 483 | ATP1B1 exon 1 |
| SEQ ID NO: 484 | ATP1B1 exon 2 |
| SEQ ID NO: 485 | ATP1B1 exon 3 |
| SEQ ID NO: 486 | ATP1B1 exon 4 |
| SEQ ID NO: 487 | ATP1B1 exon 5 |
| SEQ ID NO: 488 | ATP1B1 exon 6 |
| SEQ ID NO: 489 | ATP1B1 exon 1-6 |
| SEQ ID NO: 490 | ATP1B1 exon 1-2 |

ACTCCGAGAGCCGCAGCGGCAGCGGCGCGTCCTGCCTGCAGAGAGCCAGGCCGGAGAAGCCGAG
CGGCGCAGAGGACGCCAGGGCGCGCGCCGCAGCCACCCACCCTCCGGACCGCGGCAGCTGCTGAC
CCGCCATCGCCATGGCCCGCGGGAAAGCCAAGGAGGAGGGCAGCTGGAAGAAATTCATCTGGAAC
TCAGAGAAGAAGGAGTTTCTGGGCAGGACCGGTGGCAGTTGGTTTAAGATCCTTCTATTCTACGTAA
TATTTTATGGCTGCCTGGCTGGCATCTTCATCGGAACCATCCAAGTGATGCTGCTCACCATCAGTGA
ATTTAAGCCCACATATCAGGACCGAGTGGCCCCGCCAGGATTAACACAGATTCCTCAGATCCAGAAG
ACTGAAATTTCCTTTCGTCCTAATGATCCCAAGAGCTATGAGGCATATGTACTGAACATAGTTAGGTT
CCTGGAAAAGTACAAAGATTCAGCCCAGAGGGATGACATGATTTTTGAAGATTGTGGCGATGTGCCC
AGTGAACCGAAAGAACGAGGAGACTTTAATCATGAACGAGGAGAGCGAAAGGTCTGCAGATTCAAG
CTTGAATGGCTGGGAAATTGCTCTGGATTAAATGATGAAACTTATGGCTACAAAGAGGGCAAACCGT
GCATTATTATAAAGCTCAACCGAGTTCTAGGCTTCAAACCTAAGCCTCCCAAGAATGAGTCCTTGGA
GACTTACCCAGTGATGAAGTATAACCCAAATGTCCTTCCCGTTCAGTGCACTGGCAAGCGAGATGAA
GATAAGGATAAAGTTGGAAATGTGGAGTATTTTGGACTGGGCAACTCCCCTGGTTTTCCTCTGCAGT
ATTATCCGTACTATGGCAAACTCCTGCAGCCCAAATACCTGCAGCCCCTGCTGGCCGTACAGTTCAC
CAATCTTACCATGGACACTGAAATTCGCATAGAGTGTAAGGCGTACGGTGAGAACATTGGGTACAGT
GAGAAAGACCGTTTTCAGGGACGTTTTGATGTAAAAATTGAAGTTAAGAGCTGATCACAAGCACAAA
TCTTTCCCACTAGCCATTTAATAAGTTAAAAAAAGATACAAAAACAAAAACCTACTAGTCTTGAACAAA
CTGTCATACGTATGGGACCTACACTTAATCTATATGCTTTACACTAGCTTTCTGCATTTAATAGGTTAG
AATGTAAATTAAAGTGTAGCAATAGCAACAAAATATTTATTCTACTGTAAATGACAAAAGAAAAAGAA
AAATTGAGCCTTGGGACGTGCCCATTTTTACTGTAAATTATGATTCCGTAACTGACTTGTAGTAAGCA
GTGTTTCTGGCCCCTAAGTATTGCTGCCTTGTGTATTTTATTTAGTGTACAGTACTACAGGTGCATAC
TCTGGTCATTTTTCAAGCCATGTTTTATTGTATCTGTTTTCTACTTTATGTGAGCAAGGTTTGCTGTCC
AAGGTGTAAATATTCAACGGGAATAAAACTGGCATGGTAATTTTTTTTTTTTTTTTTTTTTTGTTTTTTG
GCTCTTTCAAAGGTAATGGCCCATCGATGAGCATTTTTAACATACTCCATAGTCTTTTCCTGTGGTGT
TAGGTCTTTATTTTTATTTTTTTCCTGGGGGCTGGGGTGGGGGTTTGTCATGGGGGAACTGCCCTTT
AAATTTTAAGTGACACTACAGAAAAACACAAAAAGGTGATGGGTTGTGTTATGCTTGTATTGAATGCT
GTCTTGACATCTCTTGCCTTGTCCTCCGGTATGTTCTAAAGCTGTGTCTGAGATCTGGATCTGCCCAT
CACTTTGGCTAGTGACAGGGCTAATTAATTTGCTTTATACATTTTTTTTACTTTCCTTTTTTCCTTTCT
GGAGGCATCACATGCTGGTGCTGTGTCTTTATGAATGTTTTAACCATTTTCATGGTGGAAGAATTTTA
TATTTATGCAGTTGTACAATTTTATTTTTTTCTGCAAGAAAAAGTGTAATGTATGAAATAAACCAAAGT
CACTTGTTTGAAAATAAATCTTTATTTTGAACTTTATAAAAAGCAATGCAGTACCCCATAGACTGGTGT
TAAATGTTGTCTACAGTGCAAAATCCATGTTCTAACATATGTAATAATTGCCAGGAGTACAGTGCTCT
TGTTGATCTTGTATTCAGTCAGGTTAAAACAACGGACAATAAAAGAATGAACACA

1. A method for determining the presence or absence of an NRG1 fusion polynucleotide in a sample, the method comprising:
1) providing a liquid biopsy sample obtained or derived from a subject having a solid tumor or a solid cancer, and
2) determining the presence or absence of the NRG1 fusion polynucleotide in the sample,
wherein the NRG1 fusion polynucleotide is a fusion of an NRG1 polynucleotide with a second polynucleotide, which second polynucleotide does not encode NRG1, and wherein the NRG1 fusion polynucleotide is RNA.

2. The method of claim 1, wherein the second polynucleotide is located 5′ to the NRG1 polynucleotide in the NRG1 fusion polynucleotide.

3. The method of claim 1, wherein the NRG1 polynucleotide comprises an EGF-like domain.

4. The method of claim 1, wherein the fusion between the NRG1 polynucleotide and the second polynucleotide is an in-frame fusion.

5. The method of claim 1, wherein the second polynucleotide is selected from a PVALB polynucleotide, a VAPB polynucleotide, a CADM1 polynucleotide, a CD44 polynucleotide or a SLC3A2 polynucleotide, a VTCN1 polynucleotide, a CDH1 polynucleotide, a CXADR polynucleotide, a GTF2E2 polynucleotide, a CSMD1 polynucleotide, a PTN polynucleotide, a ST14 polynucleotide, THBS1 polynucleotide, and a AGRN polynucleotide, an APP polynucleotide, a WRN polynucleotide, a DAAM1 polynucleotide, an ASPH polynucleotide, a NOTCH2 polynucleotide, a CD74 polynucleotide, a SDC4 polynucleotide, a SLC4A4 polynucleotide, a ZFAT polynucleotide and a DSCAML1 polynucleotide.

6. The method of claim 1, wherein the NRG1 polynucleotide comprises (i) the sequence of SEQ ID NO: 14, (ii) a sequence having at least about 70% sequence identity to SEQ ID NO: 14, or (iii) a fragment comprising at least about 20 contiguous nucleotides of (i) or (ii).

7. The method of claim 5, wherein
(a) the PVALB polynucleotide comprises (i) the sequence of SEQ ID NO: 223, (ii) a sequence having at least about 70% sequence identity to SEQ ID NO: 223, or (iii) a fragment comprising at least about 20 contiguous nucleotides of (i) or (ii);
(b) the CADM1 polynucleotide comprises (i) the sequence of SEQ ID NO: 39, (ii) a sequence having at least about 70% sequence identity to SEQ ID NO: 39, or (iii) a fragment comprising at least about 20 contiguous nucleotides of (i) or (ii);
(c) the CD44 polynucleotide comprises (i) the sequence of SEQ ID NO: 60, (ii) a sequence having at least about 70% sequence identity to SEQ ID NO: 60, or (iii) a fragment comprising at least about 20 contiguous nucleotides of (i) or (ii);
(d) the SLC3A2 polynucleotide comprises (i) the sequence of SEQ ID NO: 72, (ii) a sequence having at least about 70% sequence identity to SEQ ID NO: 72, or (iii) a fragment comprising at least about 20 contiguous nucleotides of (i) or (ii);
(e) the VTCN1 polynucleotide comprises (i) the sequence of SEQ ID NO: 81, (ii) a sequence having at least about 70% sequence identity to SEQ ID NO: 81, or (iii) a fragment comprising at least about 20 contiguous nucleotides of (i) or (ii);
(f) the CDH1 polynucleotide comprises (i) the sequence of SEQ ID NO: 100, (ii) a sequence having at least about 70% sequence identity to SEQ ID NO: 100, or (iii) a fragment comprising at least about 20 contiguous nucleotides of (i) or (ii);
(g) the CXADR polynucleotide comprises (i) the sequence of SEQ ID NO: 108, (ii) a sequence having at least about 70% sequence identity to SEQ ID NO: 108, or (iii) a fragment comprising at least about 20 contiguous nucleotides of (i) or (ii);
(h) the GTF2E2 polynucleotide comprises (i) the sequence of SEQ ID NO: 118, (ii) a sequence having at least about 70% sequence identity to SEQ ID NO: 118, or (iii) a fragment comprising at least about 20 contiguous nucleotides of (i) or (ii);
(i) the CSMD1 polynucleotide comprises (i) the sequence of SEQ ID NO: 144, (ii) a sequence having at least about 70% sequence identity to SEQ ID NO: 144, or (iii) a fragment comprising at least about 20 contiguous nucleotides of (i) or (ii);
(j) the PTN polynucleotide comprises (i) the sequence of SEQ ID NO: 152, (ii) a sequence having at least about 70% sequence identity to SEQ ID NO: 152, or (iii) a fragment comprising at least about 20 contiguous nucleotides of (i) or (ii);
(k) the ST14 polynucleotide comprises (i) the sequence of SEQ ID NO: 174, (ii) a sequence having at least about 70% sequence identity to SEQ ID NO: 174, or (iii) a fragment comprising at least about 20 contiguous nucleotides of (i) or (ii);
(l) the THBS1 polynucleotide comprises (i) the sequence of SEQ ID NO: 199, (ii) a sequence having at least about 70% sequence identity to SEQ ID NO: 199, or (iii) a fragment comprising at least about 20 contiguous nucleotides of (i) or (ii);
(m) the AGRN polynucleotide comprises (i) the sequence of SEQ ID NO: 215, (ii) a sequence having at least about 70% sequence identity to SEQ ID NO: 215, or (iii) a fragment comprising at least about 20 contiguous nucleotides of (i) or (ii);
(n) the VAPB polynucleotide comprises (i) the sequence of SEQ ID NO: 26, (ii) a sequence having at least about 70% sequence identity to SEQ ID NO: 26, or (iii) a fragment comprising at least about 20 contiguous nucleotides of (i) or (ii);
(o) the SLC3A2 polynucleotide comprises (i) the sequence of SEQ ID NO: 238, (ii) a sequence having at least about 70% sequence identity to SEQ ID NO: 238, or (iii) a fragment comprising at least about 20 contiguous nucleotides of (i) or (ii);
(p) the APP polynucleotide comprises (i) the sequence of SEQ ID NO: 258, (ii) a sequence having at least about 70% sequence identity to SEQ ID NO: 258, or (iii) a fragment comprising at least about 20 contiguous nucleotides of (i) or (ii);
(q) the WRN polynucleotide comprises (i) the sequence of SEQ ID NO: 296, (ii) a sequence having at least about 70% sequence identity to SEQ ID NO: 296, or (iii) a fragment comprising at least about 20 contiguous nucleotides of (i) or (ii);
(r) the DAAM1 polynucleotide comprises (i) the sequence of SEQ ID NO: 324, (ii) a sequence having at least about 70% sequence identity to SEQ ID NO: 324, or (iii) a fragment comprising at least about 20 contiguous nucleotides of (i) or (ii);
(s) the ASPH polynucleotide comprises (i) the sequence of SEQ ID NO: 351, (ii) a sequence having at least about 70% sequence identity to SEQ ID NO: 351, or (iii) a fragment comprising at least about 20 contiguous nucleotides of (i) or (ii); or
(t) the NOTCH2 polynucleotide comprises (i) the sequence of SEQ ID NO: 362, (ii) a sequence having at least about 70% sequence identity to SEQ ID NO: 362, or (iii) a fragment comprising at least about 20 contiguous nucleotides of (i) or (ii);
(u) the CD74 polynucleotide comprises (i) the sequence of SEQ ID NO: 374, (ii) a sequence having at least about 70% sequence identity to SEQ ID NO: 374, or (iii) a fragment comprising at least about 20 contiguous nucleotides of (i) or (ii);
(v) the SDC4 polynucleotide comprises (i) the sequence of SEQ ID NO: 382, (ii) a sequence having at least about 70% sequence identity to SEQ ID NO: 382, or (iii) a fragment comprising at least about 20 contiguous nucleotides of (i) or (ii);
(w) the SLC4A4 polynucleotide comprises (i) the sequence of SEQ ID NO: 411, (ii) a sequence having at least about 70% sequence identity to SEQ ID NO: 411, or (iii) a fragment comprising at least about 20 contiguous nucleotides of (i) or (ii);
(x) the ZFAT polynucleotide comprises (i) the sequence of SEQ ID NO: 430, (ii) a sequence having at least about 70% sequence identity to SEQ ID NO: 430, or (iii) a fragment comprising at least about 20 contiguous nucleotides of (i) or (ii); or
(y) the DSCAML1 polynucleotide comprises (i) the sequence of SEQ ID NO: 466, (ii) a sequence having at least about 70% sequence identity to SEQ ID NO: 466, or (iii) a fragment comprising at least about 20 contiguous nucleotides of (i) or (ii).

8. The method of claim 1, wherein the NRG1 fusion polynucleotide is:
(a) a PVALB-NRG1 fusion polynucleotide which comprises (i) the sequence of SEQ ID NO: 217, (ii) a sequence having at least about 70% sequence identity to SEQ ID NO: 217, or (iii) a fragment comprising at least about 20 contiguous nucleotides of (i) or (ii), including at least the nucleotides at positions 102 and 103 of SEQ ID NO: 217;
(b) an CADM1-NRG1 fusion polynucleotide which comprises (i) the sequence of SEQ ID NO: 27, (ii) a sequence having at least about 70% sequence identity to SEQ ID NO: 27, or (iii) a fragment comprising at least about 20 contiguous nucleotides of (i) or (ii), including at least the nucleotides at positions 53 and 54 of SEQ ID NO: 27;
(c) an CD44-NRG1 fusion polynucleotide which comprises (i) the sequence of SEQ ID NO: 41, (ii) a sequence having at least about 70% sequence identity to SEQ ID NO: 41, or (iii) a fragment comprising at least about 20 contiguous nucleotides of (i) or (ii), including at least the nucleotides at positions 52 and 53 of SEQ ID NO: 41;
(d) an SLC3A2-NRG1 fusion polynucleotide which comprises (i) the sequence of SEQ ID NO: 62, (ii) a sequence having at least about 70% sequence identity to SEQ ID NO: 62, or (iii) a fragment comprising at least about 20 contiguous nucleotides of (i) or (ii), including at least the nucleotides at positions 53 and 54 of SEQ ID NO: 62;
(e) an VTCN1-NRG1 fusion polynucleotide which comprises (i) the sequence of SEQ ID NO: 74, (ii) a sequence having at least about 70% sequence identity to SEQ ID NO: 74, or (iii) a fragment comprising at least about 20 contiguous nucleotides of (i) or (ii), including at least the nucleotides at positions 65 and 66 of SEQ ID NO: 74;
(f) an CDH1-NRG1 fusion polynucleotide which comprises (i) the sequence of SEQ ID NO: 83, (ii) a sequence having at least about 70% sequence identity to SEQ ID NO: 83, or (iii) a fragment comprising at least about 20 contiguous nucleotides of (i) or (ii), including at least the nucleotides at positions 119 and 120 of SEQ ID NO: 83;
(g) a CXADR-NRG1 fusion polynucleotide which comprises (i) the sequence of SEQ ID NO: 102, (ii) a sequence having at least about 70% sequence identity to SEQ ID NO: 102, or (iii) a fragment comprising at least about 20 contiguous nucleotides of (i) or (ii), including at least the nucleotides at positions 43 and 44 of SEQ ID NO: 102;
(h) a GTF2E2-NRG1 fusion polynucleotide which comprises (i) the sequence of SEQ ID NO: 109, (ii) a sequence having at least about 70% sequence identity to SEQ ID NO: 109, or (iii) a fragment comprising at least about 20 contiguous nucleotides of (i) or (ii), including at least the nucleotides at positions 141 and 142 of SEQ ID NO: 109;
(i) a CSMD1-NRG1 fusion polynucleotide which comprises (i) the sequence of SEQ ID NO: 120, (ii) a sequence having at least about 70% sequence identity to SEQ ID NO: 120, or (iii) a fragment comprising at least about 20 contiguous nucleotides of (i) or (ii), including at least the nucleotides at positions 88 and 89 of SEQ ID NO: 120;
(j) a PTN-NRG1 fusion polynucleotide which comprises (i) the sequence of SEQ ID NO: 146, (ii) a sequence having at least about 70% sequence identity to SEQ ID NO: 146, or (iii) a fragment comprising at least about 20 contiguous nucleotides of (i) or (ii), including at least the nucleotides at positions 102 and 103 of SEQ ID NO: 146;
(k) a ST14-NRG1 fusion polynucleotide which comprises (i) the sequence of SEQ ID NO: 154, (ii) a sequence having at least about 70% sequence identity to SEQ ID NO: 154, or (iii) a fragment comprising at least about 20 contiguous nucleotides of (i) or (ii), including at least the nucleotides at positions 95 and 96 of SEQ ID NO: 154;
(l) a THBS1-NRG1 fusion polynucleotide which comprises (i) the sequence of SEQ ID NO: 176, (ii) a sequence having at least about 70% sequence identity to SEQ ID NO: 176, or (iii) a fragment comprising at least about 20 contiguous nucleotides of (i) or (ii), including at least the nucleotides at positions 56 and 57 of SEQ ID NO: 176;
(m) an AGRN-NRG1 fusion polynucleotide which comprises (i) the sequence of SEQ ID NO: 201, (ii) a sequence having at least about 70% sequence identity to SEQ ID NO: 201, or (iii) a fragment comprising at least about 20 contiguous nucleotides of (i) or (ii), including at least the nucleotides at positions 106 and 107 of SEQ ID NO: 201;
(n) an VAPB-NRG1 fusion polynucleotide which comprises (i) the sequence of SEQ ID NO: 19, (ii) a sequence having at least about 70% sequence identity to SEQ ID NO: 19, or (iii) a fragment comprising at least about 20 contiguous nucleotides of (i) or (ii), including at least the nucleotides at positions 43 and 44 of SEQ ID NO: 19;
(o) an SLC3A2-NRG1 fusion polynucleotide which comprises (i) the sequence of SEQ ID NO: 225, (ii) a sequence having at least about 70% sequence identity to SEQ ID NO:225, or (iii) a fragment comprising at least about 20 contiguous nucleotides of (i) or (ii), including at least the nucleotides at positions 93 and 94 of SEQ ID NO: 225;
(p) an APP-NRG1 fusion polynucleotide which comprises (i) the sequence of SEQ ID NO: 240, (ii) a sequence having at least about 70% sequence identity to SEQ ID NO: 240, or (iii) a fragment comprising at least about 20 contiguous nucleotides of (i) or (ii), including at least the nucleotides at positions 54 and 55 of SEQ ID NO: 240;
(q) a WRN-NRG1 fusion polynucleotide which comprises (i) the sequence of SEQ ID NO: 260, (ii) a sequence having at least about 70% sequence identity to SEQ ID NO: 260, or (iii) a fragment comprising at least about 20 contiguous nucleotides of (i) or (ii), including at least the nucleotides at positions 96 and 97 of SEQ ID NO: 260;
(r) a DAAM1-NRG1 fusion polynucleotide which comprises (i) the sequence of SEQ ID NO: 298, (ii) a sequence having at least about 70% sequence identity to SEQ ID NO: 298, or (iii) a fragment comprising at least about 20 contiguous nucleotides of (i) or (ii), including at least the nucleotides at positions 75 and 76 of SEQ ID NO: 298;
(s) an ASPH-NRG1 fusion polynucleotide which comprises (i) the sequence of SEQ ID NO: 325, (ii) a sequence having at least about 70% sequence identity to SEQ ID NO: 325, or (iii) a fragment comprising at least about 20 contiguous nucleotides of (i) or (ii), including at least the nucleotides at positions 75 and 76 of SEQ ID NO: 325;
(t) a NOTCH2-NRG1 fusion polynucleotide which comprises (i) the sequence of SEQ ID NO: 353, (ii) a sequence having at least about 70% sequence identity to SEQ ID NO: 353, or (iii) a fragment comprising at least about 20 contiguous nucleotides of (i) or (ii), including at least the nucleotides at positions 75 and 76 of SEQ ID NO: 353;
(u) a CD74-NRG1 fusion polynucleotide which comprises (i) the sequence of SEQ ID NO: 364, (ii) a sequence having at least about 70% sequence identity to SEQ ID NO: 364, or (iii) a fragment comprising at least about 20 contiguous nucleotides of (i) or (ii), including at least the nucleotides at positions 75 and 76 of SEQ ID NO: 364;
(v) a SDC4-NRG1 fusion polynucleotide which comprises (i) the sequence of SEQ ID NO: 376 or 468, (ii) a sequence having at least about 70% sequence identity to SEQ ID NO: 376 or 468, or (iii) a fragment comprising at least about 20 contiguous nucleotides of (i) or (ii), including at least the nucleotides at positions 75 and 76 of SEQ ID NO: 376 or 468;
(w) a SLC4A4-NRG1 fusion polynucleotide which comprises (i) the sequence of SEQ ID NO: 384, (ii) a sequence having at least about 70% sequence identity to SEQ ID NO: 384, or (iii) a fragment comprising at least about 20 contiguous nucleotides of (i) or (ii), including at least the nucleotides at positions 75 and 76 of SEQ ID NO: 384;
(x) a ZFAT-NRG1 fusion polynucleotide which comprises (i) the sequence of SEQ ID NO: 413, (ii) a sequence having at least about 70% sequence identity to SEQ ID NO: 413, or (iii) a fragment comprising at least about 20 contiguous nucleotides of (i) or (ii), including at least the nucleotides at positions 75 and 76 of SEQ ID NO: 413;
(y) a DSCAML1-NRG1 fusion polynucleotide which comprises (i) the sequence of SEQ ID NO: 432, (ii) a sequence having at least about 70% sequence identity to SEQ ID NO: 432, or (iii) a fragment comprising at least about 20 contiguous nucleotides of (i) or (ii), including at least the nucleotides at positions 75 and 76 of SEQ ID NO: 432; or
(z) a CD44-NRG1 fusion polynucleotide which comprises (i) the sequence of SEQ ID NO: 469, (ii) a sequence having at least about 70% sequence identity to SEQ ID NO: 469, or (iii) a fragment comprising at least about 20 contiguous nucleotides of (i) or (ii), including at least the nucleotides at positions 75 and 76 of SEQ ID NO: 469.

9. The method of claim 1, wherein the NRG1 fusion polynucleotide comprises the sequence of SEQ ID NO: 19, 27, 41, 62, 74, 83, 102, 109, 120, 146, 154, 176, 201, 217, 225, 240, 260, 298, 325, 353, 364, 376, 384, 413, 432, 468 or 469.

10. (canceled)

11. The method of claim 1, wherein the method further comprises isolating from the sample one or more of cell free polynucleotide, cell-free DNA (cfDNA), cell-free RNA (cfRNA), circulating tumor RNA (ctRNA), circulating tumor cells (CTC), vesicular RNA, exosomes, extracellular vesicles and tumor-educated platelets.

12. The method of claim 1, wherein the method further comprises amplifying the NRG1 fusion polynucleotide if present in the sample.

13. The method of claim 1, wherein the presence or absence of the NRG1 fusion polynucleotide is determined by sequencing the NRG1 fusion polynucleotide or using a polynucleotide probe which specifically hybridises to the NRG1 fusion polynucleotide.

14. The method of claim 1, wherein the sample is obtained from a subject which, with the exception of the NRG1 fusion polynucleotide, does not comprise any further oncogenic mutation.

15. (canceled)

16. The method of claim 1, wherein the sample is obtained from a subject having a cancer selected from an adenocarcinoma, mucinous adenocarcinoma, a pancreatic cancer, a pancreatic adenocarcinoma, or a renal cell carcinoma, a pancreatic ductal adenocarcinoma, a sarcoma, a bladder cancer, a colon cancer, a rectal cancer, a colorectal cancer, a gallbladder cancer, a head and neck cancer, a prostate cancer, a uterus cancer, a breast cancer, an ovarian cancer, a liver cancer, an endometrial cancer, a lung cancer, in particular a non-small cell lung cancer or an invasive mucinous adenocarcinoma.

17. The method of claim 1, wherein the sample is obtained from a subject having a primary cancer or primary tumor, a recurrent cancer or recurrent tumor or a metastasized cancer or metastasized tumor.

18. The method of claim 1, wherein the sample is obtained from a human subject.

19. The method of claim 1, wherein the sample is selected from blood, blood serum, blood plasma, pleural effusion, saliva, urine, semen, sputum, vaginal fluid, amniotic fluid, peritoneal fluid, cerebrospinal fluid, bone marrow, cell free lavage and another biofluid.

20. The method of claim 1, wherein the volume of the sample is from about 0.1 to about 12 ml.

21. The method of claim 1, wherein if the NRG1 fusion polynucleotide is present, determining its amount or concentration.

22. A method for determining the presence or absence of two or more NRG1 fusion polynucleotides in a sample, the method comprising:
1) providing a liquid biopsy sample, and
2) determining the presence or absence of the two or more NRG1 fusion polynucleotides in the sample, wherein the NRG1 fusion polynucleotides are RNA polynucleotides, and wherein the NRG fusion polynucleotides are fusions of an NRG1 polynucleotide with a second polynucleotide, which second polynucleotide does not encode NRG1.

23. A kit for determining the presence or absence of an NRG1 fusion polynucleotide in a liquid biopsy sample, comprising (a) a pair of forward and reverse polynucleotide primers which specifically hybridise to the NRG1 fusion polynucleotide and/or (b) a polynucleotide probe which specifically hybridises to the NRG1 fusion polynucleotide.

24. The method of claim 1, wherein the presence or absence of an NRG1 fusion polynucleotide in a sample is used in determining whether or not a subject has cancer.

25. The method of claim 24, wherein comprising administering to the subject a therapeutically effective amount of a cancer treatment and thereby treating the cancer in the subject.

26. The method of claim 24, wherein the cancer treatment is a substance or composition.


SEQUENCE LISTING (491 sequences)

SEQ ID NO: 1 - DNA, 187 bp, Homo sapiens
agtaagcctccgcagcccactcggactgcagcctgtttgccgcccgtcctcccattgcagcactcggggcgacagagagggaggaggcgcgcggggacggggacgcccaggaggacccactcgcgggtcccgctccgctccggcagcagcatggggaaaggacgcgcgggccgagttggcaccacag

SEQ ID NO: 2 - DNA, 178 bp, Homo sapiens
ccttgcctccccgattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagg

SEQ ID NO: 3 - DNA, 122 bp, Homo sapiens
gaagtcagaacttcgcattaacaaagcatcactggctgattctggagagtatatgtgcaaagtgatcagcaaattaggaaatgacagtgcctctgccaatatcaccatcgtggaatcaaacg

SEQ ID NO: 4 - DNA, 51 bp, Homo sapiens
agatcatcactggtatgccagcctcaactgaaggagcatatgtgtcttcag

SEQ ID NO: 5 - DNA, 51 bp, Homo sapiens
agtctcccattagaatatcagtatccacagaaggagcaaatacttcttcat

SEQ ID NO: 6 - DNA, 130 bp, Homo sapiens
ctacatctacatccaccactgggacaagccatcttgtaaaatgtgcggagaaggagaaaactttctgtgtgaatggaggggagtgcttcatggtgaaagacctttcaaacccctcgagatacttgtgcaa

SEQ ID NO: 7 - DNA, 59 bp, Homo sapiens
gtgcccaaatgagtttactggtgatcgctgccaaaactacgtaatggccagcttctaca

SEQ ID NO: 8 - DNA, 24 bp, Homo sapiens
agcatcttgggattgaatttatgg

SEQ ID NO: 9 - DNA, 103 bp, Homo sapiens
aggcggaggagctgtaccagaagagagtgctgaccataaccggcatctgcatcgccctccttgtggtcggcatcatgtgtgtggtggcctactgcaaaaccaa

SEQ ID NO: 10 - DNA, 127 bp, Homo sapiens
gaaacagcggaaaaagctgcatgaccgtcttcggcagagccttcggtctgaacgaaacaatatgatgaacattgccaatgggcctcaccatcctaacccaccccccgagaatgtccagctggtgaat

SEQ ID NO: 11 - DNA, 131 bp, Homo sapiens
caatacgtatctaaaaacgtcatctccagtgagcatattgttgagagagaagcagagacatccttttccaccagtcactatacttccacagcccatcactccactactgtcacccagactcctagccacag

SEQ ID NO: 12 - DNA, 207 bp, Homo sapiens
ctggagcaacggacacactgaaagcatcctttccgaaagccactctgtaatcgtgatgtcatccgtagaaaacagtaggcacagcagcccaactgggggcccaagaggacgtcttaatggcacaggaggccctcgtgaatgtaacagcttcctcaggcatgccagagaaacccctgattcctaccgagactctcctcatagtgaaag

SEQ ID NO: 13 - DNA, 10299 bp, Homo sapiens
gtatgtgtcagccatgaccaccccggctcgtatgtcacctgtagatttccacacgccaagctcccccaaatcgcccccttcggaaatgtctccacccgtgtccagcatgacggtgtccatgccttccatggcggtcagccccttcatggaagaagagagacctctacttctcgtgacaccaccaaggctgcgggagaagaagtttgaccatcaccctcagcagttcagctccttccaccacaaccccgcgcatgacagtaacagcctccctgctagccccttgaggatagtggaggatgaggagtatgaaacgacccaagagtacgagccagcccaagagcctgttaagaaactcgccaatagccggcgggccaaaagaaccaagcccaatggccacattgctaacagattggaagtggacagcaacacaagctcccagagcagtaactcagagagtgaaacagaagatgaaagagtaggtgaagatacgcctttcctgggcatacagaaccccctggcagccagtcttgaggcaacacctgccttccgcctggctgacagcaggactaacccagcaggccgcttctcgacacaggaagaaatccaggccaggctgtctagtgtaattgctaaccaagaccctattgctgtataaaacctaaataaacacatagattcacctgtaaaactttattttatataataaagtattccaccttaaattaaacaatttattttattttagcagttctgcaaatagaaaacaggaaaaaaacttttataaattaaatatatgtatgtaaaaatgtgttatgtgccatatgtagcaattttttacagtatttcaaaacgagaaagatatcaatggtgcctttatgttatgttatgtcgagagcaagttttgtacagttacagtgattgcttttccacagtatttctgcaaaacctctcatagattcagtttttgctggcttcttgtgcattgcattatgatgttgactggatgtatgatttgcaagacttgcaactgtccctctgtttgcttgtagtagcacccgatcagtatgtcttgtaatggcacatccatccagatatgcctctcttgtgtatgaagttttctttgctttcagaatatgaaatgagttgtgtctactctgccagccaaaggtttgcctcattgggctctgagataatagtagatccaacagcatgctactattaaatacagcaagaaactgcattaagtaatgttaaatattaggaagaaagtaatactgtgatttaaaaaaaactatattattaatcagaagacagcttgctcttactaaaaggagctctcatttactttatttgattttatttttcttgacaaaaagcaacagttttagggatagcttagaaaatgggttctggcttgctatcagggtaaatctaacaccttacaagaggactgagtgtcactttctctctgggggaatgatccagcagcttatctagttgacaatcaaaacacggctgataaaggtgcaatcatttctgacatgtatttttcactgattttgaagctagtgattggttgtgtcttcttggctcaaaaagaagcatattacggcacaaaaagcccagcccagacagcacatgcagcattttgtctgaaatacttctagagtcaaacgtgcctgctgtacatagcgatgacttgtcatcatagggaagtatttccatcgtagagtgttcagaaggagtgactgtataggtggagagaagcttagtgactccgttgaaattttaaaatgtggatgaccacccctttctcccccttatttttcttttatctttccatgttgccttgatcaggtcataactatgcatgaacattttttatcaggaatggccgatgtgtatgtgatttgtaatcacaagtaatgattcatcaggaaatgtcaatcctgttggaaagattgcacctttacttgcagaagtgacccccacctgtgtcctgacctctccatttacaggctctctcacccatttcccccacctcctttaatttttgctttactgtcataaagtaggactaagattggtctaagcattgcatgttcttttgtgatggtaaatccaaaggaaggcctataagtattaacatttgaaataactgctaattcaggaaaatggaagaaaaaaaattatttgaaacacagaacccatttcatggcctgcctgatatctgtgaaatcagggctggagctttacttaggattcacatggcctcctaggaaccatgggacaaatgggaaacaggttatcgggggattcatgaagtcagtgagagtaattgcttcttttttgcgggtgaactgaatgtatttcttcaccaaatcttgatgttaacaattaaaaagaagaaatgacatgcaagtaggtcttagcagaaaaatgcaggctgggcatgagtcatgttgttaccctcccacatgctcctacaatccacagagatgcctgtctgcaggttcttgaagttattgttagtatttggtatctcaaatttttcgtcactgttcacatgccactttctctgtgcacagtggtatcctcatttgctttttaacctacactgaggagtctttgtcaggttgcactgattttccaattctgcagtaatgagtaagctcacggcatggggaagaagacagtcagtccaatgaagttctctaaattattttaacattgcctttgaaggccttgactcatccttagctatttcaatgaagaaattcctaccatgaatttaaaaccctaaaaattctgtttcaaattctttgggcattggggtactcagatatcccattgtggaagaattttaagaataaatagaagtttctgttgagaaccatgagcaacatgtttcttacaatgagaattgctatgcattttaaaattgcaaatatatatgaaaattgaagacaagaggaaattgtatttctaacttgattctgatcactcacagaggtggcatattattatagttgggacatcctttgcacccttcataaaaaaggccagctgactgctcagcatcacctgccaaggccactagatttgtgtttacaggggtatctctgtgatgcttgtcacatcactcttgaccacctctgttaataaattccgacagtgcagtggcgatcggagtgtgaacttatgttcccagcatatggaaagctatcttaggttttaaggtagtagaaattgcccaggagtttgacagcaactttgtttcccgggtctaaaatcgtatcccactgaggtgtatgcagtggagcataatacatgcaaatacatgcaaaactccttttgtttcacctaagattcactttctatcttactttcccttcctgcctagtgtgacttttgcccccaagagtgcctggacagcattctagtttctacaaaatggtcctctgtgtaggtgaatgtgtcccaaacctgctatcactttcttgtttcagtgtgactgtcttgttagaggtgaagtttatccagggtaacttgctcactaactattcctttttatggcctggggttaaagggcgcatggctcacactggtgaaaataaggaaggcctggtcttatcttgtattaataatactggctgcattccaccagccagagatttctatctgcgaagacctatgaaacactgaagagaaatgtaggcagaaggaaatggccacatatcacaagttctattatatattcttttgtaaatacatattgtatattacttggatgttttcttatatcatttactgtctttttgagttaatgtcagtttttactctctcaacttactatgtaacattgtaaataacataatgtcctttattatttatatttaagcatctaacatatagagttgttttcatataagtttaagataaatgtcaaaaatatatgttcttttgtttttctttgctttaaaattatgtatcttttccttttcttttttttaagaataatttattgttcaggagaaagaatgtatatgtaactgaaactatctgaagaatgcacattgaaggccgtgaggtactgataaactaaagaatttattattcaaaatactaagcaataagtaattgtgatttatttaaagttttgtccattttccatgaaagacatactgcaataaaaatgctactctgtggagacctgggagtgttgctcagcagactacagcttcagtctgttagaccagcacctttcatctcattcccatagttatgctaatttaggattgtgttccatggaccccatgatcaccttgtctatacgttgcttcttgtctgtccattgcttttgccacaccacctgttctcaaatcatctcctccctaccaatgctgtttatcactttcttccttgttgaagaggccacacaaccagacagtactatgcttcctttttcctccatacacaataacagagagagaatattctagggcatgactgcctggatcctggctgttgctatcttttgtagtggcagtaagaaactccttcagactaatgaaaatgtcaacgtgccattcaatcacgaaaggtaacgaaaaatgctctcatggttcaaatagtccaatggcccatagtggcctaaaaggcagccagttgacacctggccatgctaagcttccttataccatccgctaatgactttccattgggcccacaatttacggattcataattttaaaagaggagaaggccaagttaggttcattccccttattctgtcaataaaacaaatcaaactcatgtctatctaactgctcagggaggagcctttgcatgagaaaattctcatattctaagactgagtcatagaaatgagggtattacttttcttactgcaattaacctaaacaaaagccatattttaacaaatagatatttgcatggtacccttcatatattccaagcatttcactcattattccaggtaggttaagagcttctgaagtgtatgaagtaaaggtcagcaatcctttggggtgaacagtggcctcctttggagtttgggggtaacctgagacttcccaccaatgtccacctccatctgtgtacctaattcctattacctagttatggctcctctaggatcatttccaaacactctggatgtccaggaaatttaaattgtagcttttgactgagctagtttttcctatttatattaataaattttcaaaaatgcttgaaatcttcacatttgcaacaactttagttttcatgcacatacaaacacagagagacaaaaattccaaacagacactctccaaaagccaccacactctttcacttgctctatagtcatttagccaaccagccatgcagagaatatttaaaacttaaagattgagacattattctcagtttttgctgaggctttgtaacgaaattgaacactataagcagctattgtagtaattttggttaaaattgtttgcctgggatatagtatttgaggcagaagcacgtgtgtgaaggaggtgaggtggtttggaaagagtgaagactcgcagccagattgaatgtctggataattactataattctcccttcttggttgaaaccatgttctctcttgatttttaaacccaggctgcctctggaaacaagcaaacctgagtctttctaacctgagtcttcccaatcattagatttcttttctgtcctaacgatgaatgataaaaggacttgatgttcacaatttggggttataaggcaggtctgaaatctggagactcaagatgctggaaggagtggaaagtttcgatgactttatatgaatcactttgcactctatgtttggcttgtcctctttgaaactgatttactaaaataaatgtaagggaactattactccaaaagattaacttggcaggaaataccaatactttcagtttatgaaagacaaaactgtcttgttgctacaggaagctgcaatgttcctaacctttaaggttggtgttgaatagggtggtcatgccctcccctgcaggtatctttaggctcctgttgacctcctggtactataactgttcgtcttctctgggtagctattgattttgaactttaacatgcttcaaaactttattcatcagggaaataggaaaagagttttgttacctggaggaaatctattgtgatctacctgagctttttaaaaacagaccaggagaaggaaaccagtaatttttaaagaagagacagagaatgggataatagtttcacccaggatctctttctaaccctttcccttcaaatgaacttattggaacagaattggaaagaagaaaggacatctctgcccaccccacaggatgccaaaaaggctaaagaattacctctgtagatttaaacatctttaatggcttatgtatagatttgctaatacagagagaaatgaactattaaataaaaatcacattttataatatttttatggcttaaaacatcctttatctcctttttgttctctctacatgatatggtaagtgatgaggaaaatttaggctcaggaaggttaaaatctttcttggagttacacatctaagagagctgcagagctgacacttgtacccaggttttctgactgcaaatccagtttctttctattgcgttcttcccctttccctgcctcaagcagaaacaggttttttattttcaacctttatgtatacagtatgttatgttacatctacagctaagtttctttttagaagaatgtgagcccttctagctttggtttagagtgattctagaagccaatttccttggcttagtgattctatgcacctttcctaaacttagctttctaaggaaatgaagtgtacgagtgagaatgaattcacaatttcgacatgtaggtagcatcctaaagtgaaaagaggaggaaatttgtggtcaaagcactctccccaccacttagaaacttactgactgtgggcagcttcctcctccaagtttccttcctgatttacaagaccgtggtgtggtcaggattaaacttgaatacatgtaaggaagcctgaaagtgtctaacacatagcgagtattcaaatgccaccttctatttgatccttcccctccagttccttaagttttggaatctaggtttctcagttccaaatggattgacatttgcatatccccattgcacaatggatcaaataaactttatgttatcatttctccaacatagtgccagtaagcaaatcctttttaataacaacagtatgttgagaaacatatcaccaaataatatttaactttgtagctttgataagttctttaggttttggttttggttttgttttctgagacagggtcttgatctgtcacccagactggagtgcaatggttcaattttagctcactgcaacctgtaactcctgagctcaagtgatcctcccacctcagcctctcaagtagctgggactacaggtgtgctccaccatgcccagcgatttttttttttttttttttttttttttttggtagggacaaggtctcgctatgttgcccaggctggtcttcaattcctggccttaagtgatcctcctgcctcggcctcccaaagtgctggaattacaggcatgagccaccacccataactttatgtttgtttttttgatgcagtataagttcagcttgcttcttatgcagccataccatttcatgttaactctgatttttagcagcttattacattagtgttttattattaatataattttacagaaatttactaaaccatgactctgtagagttttaataatactacctccaaacatcattgcaaacatctagaagaatgaacaaaaatgatcttagatcgacagtatatctgtttgtcttagtttctacacaggatgttcagacatattccatttctttaaaaaaaaaatatatatatatatatatatatataggcctggcacggtggctcatgactgtaatcccagcactttgggaggcttaggcaggcaaatcacctgaggtcaggagtttgagactggcctgaccaacatggtgaaacctcgtctctattaaaattacaaaaattagccgggcgtggtggcacatgcctgtaatcccagctactcgggaggctgaggcaggagaatcacttgaacctgggaggcagaggttgcagtgagttgagatcacgccattgcactccagccttggtgacaagagtgaaactccgtctcaaaaaaaaaaaaaatatgtatatatatatatatatatatatatatatatatatatatatatatataaaatcccaccaaaagtctgcagagtgaccaaattagacggctctggtttcagattaaattctaaatgtgagaaaccacatagctcccatgatccatccaataattcctcacgtcctcttcactctttactccatgcataaaacagaatttttttttctcatcctgggtatgaagcaattaataatttacggatttagcctatttggattcaatcccttcaaactccatactacatccaaggtggaagtgacttaaactctgatatcaatcatcaggcttgtaatataggctttgttaatggcaggagagtctaataaaactttctgttccttatccttcatttaaatgaaaaactttttattgaaaacaatcataactctagctcatcataaatataattcatgaggacattttattatttttatattaaagaaataatattatagatgtaaactttgcacctttctaattattatcatgagttaagctaatacttgtcttctggtccctagatgatgattcttttttgccttactggaggagcccttgtcttgaagtgagttgcttcaacagcagaggacttctagtttctcccagttgagcctaaagtgaacttttcatcttcttcagaggaaggggcttccttgatttgtacttttgtggctcctcagataacacagacaattttatcttggatcccaggttctcttcaccattaagaataagaaagagagaaaatgctgtgcatgacagccacctactccaaactacccaaccccctgtaaccaggtaccttccaacaacgagatgattctgccctcactcaagagtctcccccacaaagattccattctccctttactttttattttttatttttttgcaaaacaaaggcctcctttagtacctccttagtttataacccttatcttcccagctcttcccttcactgatacctctgatttcaaaagttctgaagtcggaagaccacacaatttcagactgtgaacagaaattcagtcagaaattattggagttagaaagaatttagagaaattgtattgaactagaaagtcctctttgattagtggtggccctttagaaagttctagggcagagtcccatggtgtttcatcttttccatgatttgcttgaccaaaaacttttctttcacaacatgaaaatatgctaatccaccacacattttggattctgctctgtttgcctgaggtgttagatctctagccaggactgtgaagggaaggaacttgaatccttcctattgagctattaatgcagagtcagtgagatgaagggttccactcggggtcaaaatcatgtcagttaccaagcaaaggagcaagtaaggggaaacatctcctcatctggttagtggagccacatttcacccactgatcaagccagacctgagcaatagtctagatttctccctccacatctaattggtgacaatggtgattgtaccactgacaggtcacacaagtccacaccctccttcacagcctcactgcttcggctcttgtttatgctcttatcagcatctgtcactaggatccatttgtctcctgactcatctacttcctttcactcccctgggccatccttcacatcatgctagaagactgtgtctaacttgcagaacttattgtgtcaatctcctggttacggcccctccatggctccccacctgacataggaggcccttcacaatctggcttctgtcactcataacttgtctccagccttcattcttcagtctgattttatggttttctagttccccaatacaccacaccagtgtttatgaaaccctagtcattggcctacgaactttatgattattgacatattcatgtaccacctgtattattttttgcatagtgtttatttttaattgactacatttttaactacaataaagtaatttcaactaaaa

SEQ ID NO: 14 - DNA, 11669 bp, Homo sapiens
agtaagcctccgcagcccactcggactgcagcctgtttgccgcccgtcctcccattgcagcactcggggcgacagagagggaggaggcgcgcggggacggggacgcccaggaggacccactcgcgggtcccgctccgctccggcagcagcatggggaaaggacgcgcgggccgagttggcaccacagccttgcctccccgattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgagatcatcactggtatgccagcctcaactgaaggagcatatgtgtcttcagagtctcccattagaatatcagtatccacagaaggagcaaatacttcttcatctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacaagcatcttgggattgaatttatggaggcggaggagctgtaccagaagagagtgctgaccataaccggcatctgcatcgccctccttgtggtcggcatcatgtgtgtggtggcctactgcaaaaccaagaaacagcggaaaaagctgcatgaccgtcttcggcagagccttcggtctgaacgaaacaatatgatgaacattgccaatgggcctcaccatcctaacccaccccccgagaatgtccagctggtgaatcaatacgtatctaaaaacgtcatctccagtgagcatattgttgagagagaagcagagacatccttttccaccagtcactatacttccacagcccatcactccactactgtcacccagactcctagccacagctggagcaacggacacactgaaagcatcctttccgaaagccactctgtaatcgtgatgtcatccgtagaaaacagtaggcacagcagcccaactgggggcccaagaggacgtcttaatggcacaggaggccctcgtgaatgtaacagcttcctcaggcatgccagagaaacccctgattcctaccgagactctcctcatagtgaaaggtatgtgtcagccatgaccaccccggctcgtatgtcacctgtagatttccacacgccaagctcccccaaatcgcccccttcggaaatgtctccacccgtgtccagcatgacggtgtccatgccttccatggcggtcagccccttcatggaagaagagagacctctacttctcgtgacaccaccaaggctgcgggagaagaagtttgaccatcaccctcagcagttcagctccttccaccacaaccccgcgcatgacagtaacagcctccctgctagccccttgaggatagtggaggatgaggagtatgaaacgacccaagagtacgagccagcccaagagcctgttaagaaactcgccaatagccggcgggccaaaagaaccaagcccaatggccacattgctaacagattggaagtggacagcaacacaagctcccagagcagtaactcagagagtgaaacagaagatgaaagagtaggtgaagatacgcctttcctgggcatacagaaccccctggcagccagtcttgaggcaacacctgccttccgcctggctgacagcaggactaacccagcaggccgcttctcgacacaggaagaaatccaggccaggctgtctagtgtaattgctaaccaagaccctattgctgtataaaacctaaataaacacatagattcacctgtaaaactttattttatataataaagtattccaccttaaattaaacaatttattttattttagcagttctgcaaatagaaaacaggaaaaaaacttttataaattaaatatatgtatgtaaaaatgtgttatgtgccatatgtagcaattttttacagtatttcaaaacgagaaagatatcaatggtgcctttatgttatgttatgtcgagagcaagttttgtacagttacagtgattgcttttccacagtatttctgcaaaacctctcatagattcagtttttgctggcttcttgtgcattgcattatgatgttgactggatgtatgatttgcaagacttgcaactgtccctctgtttgcttgtagtagcacccgatcagtatgtcttgtaatggcacatccatccagatatgcctctcttgtgtatgaagttttctttgctttcagaatatgaaatgagttgtgtctactctgccagccaaaggtttgcctcattgggctctgagataatagtagatccaacagcatgctactattaaatacagcaagaaactgcattaagtaatgttaaatattaggaagaaagtaatactgtgatttaaaaaaaactatattattaatcagaagacagcttgctcttactaaaaggagctctcatttactttatttgattttatttttcttgacaaaaagcaacagttttagggatagcttagaaaatgggttctggcttgctatcagggtaaatctaacaccttacaagaggactgagtgtcactttctctctgggggaatgatccagcagcttatctagttgacaatcaaaacacggctgataaaggtgcaatcatttctgacatgtatttttcactgattttgaagctagtgattggttgtgtcttcttggctcaaaaagaagcatattacggcacaaaaagcccagcccagacagcacatgcagcattttgtctgaaatacttctagagtcaaacgtgcctgctgtacatagcgatgacttgtcatcatagggaagtatttccatcgtagagtgttcagaaggagtgactgtataggtggagagaagcttagtgactccgttgaaattttaaaatgtggatgaccacccctttctcccccttatttttcttttatctttccatgttgccttgatcaggtcataactatgcatgaacattttttatcaggaatggccgatgtgtatgtgatttgtaatcacaagtaatgattcatcaggaaatgtcaatcctgttggaaagattgcacctttacttgcagaagtgacccccacctgtgtcctgacctctccatttacaggctctctcacccatttcccccacctcctttaatttttgctttactgtcataaagtaggactaagattggtctaagcattgcatgttcttttgtgatggtaaatccaaaggaaggcctataagtattaacatttgaaataactgctaattcaggaaaatggaagaaaaaaaattatttgaaacacagaacccatttcatggcctgcctgatatctgtgaaatcagggctggagctttacttaggattcacatggcctcctaggaaccatgggacaaatgggaaacaggttatcgggggattcatgaagtcagtgagagtaattgcttcttttttgcgggtgaactgaatgtatttcttcaccaaatcttgatgttaacaattaaaaagaagaaatgacatgcaagtaggtcttagcagaaaaatgcaggctgggcatgagtcatgttgttaccctcccacatgctcctacaatccacagagatgcctgtctgcaggttcttgaagttattgttagtatttggtatctcaaatttttcgtcactgttcacatgccactttctctgtgcacagtggtatcctcatttgctttttaacctacactgaggagtctttgtcaggttgcactgattttccaattctgcagtaatgagtaagctcacggcatggggaagaagacagtcagtccaatgaagttctctaaattattttaacattgcctttgaaggccttgactcatccttagctatttcaatgaagaaattcctaccatgaatttaaaaccctaaaaattctgtttcaaattctttgggcattggggtactcagatatcccattgtggaagaattttaagaataaatagaagtttctgttgagaaccatgagcaacatgtttcttacaatgagaattgctatgcattttaaaattgcaaatatatatgaaaattgaagacaagaggaaattgtatttctaacttgattctgatcactcacagaggtggcatattattatagttgggacatcctttgcacccttcataaaaaaggccagctgactgctcagcatcacctgccaaggccactagatttgtgtttacaggggtatctctgtgatgcttgtcacatcactcttgaccacctctgttaataaattccgacagtgcagtggcgatcggagtgtgaacttatgttcccagcatatggaaagctatcttaggttttaaggtagtagaaattgcccaggagtttgacagcaactttgtttcccgggtctaaaatcgtatcccactgaggtgtatgcagtggagcataatacatgcaaatacatgcaaaactccttttgtttcacctaagattcactttctatcttactttcccttcctgcctagtgtgacttttgcccccaagagtgcctggacagcattctagtttctacaaaatggtcctctgtgtaggtgaatgtgtcccaaacctgctatcactttcttgtttcagtgtgactgtcttgttagaggtgaagtttatccagggtaacttgctcactaactattcctttttatggcctggggttaaagggcgcatggctcacactggtgaaaataaggaaggcctggtcttatcttgtattaataatactggctgcattccaccagccagagatttctatctgcgaagacctatgaaacactgaagagaaatgtaggcagaaggaaatggccacatatcacaagttctattatatattcttttgtaaatacatattgtatattacttggatgttttcttatatcatttactgtctttttgagttaatgtcagtttttactctctcaacttactatgtaacattgtaaataacataatgtcctttattatttatatttaagcatctaacatatagagttgttttcatataagtttaagataaatgtcaaaaatatatgttcttttgtttttctttgctttaaaattatgtatcttttccttttcttttttttaagaataatttattgttcaggagaaagaatgtatatgtaactgaaactatctgaagaatgcacattgaaggccgtgaggtactgataaactaaagaatttattattcaaaatactaagcaataagtaattgtgatttatttaaagttttgtccattttccatgaaagacatactgcaataaaaatgctactctgtggagacctgggagtgttgctcagcagactacagcttcagtctgttagaccagcacctttcatctcattcccatagttatgctaatttaggattgtgttccatggaccccatgatcaccttgtctatacgttgcttcttgtctgtccattgcttttgccacaccacctgttctcaaatcatctcctccctaccaatgctgtttatcactttcttccttgttgaagaggccacacaaccagacagtactatgcttcctttttcctccatacacaataacagagagagaatattctagggcatgactgcctggatcctggctgttgctatcttttgtagtggcagtaagaaactccttcagactaatgaaaatgtcaacgtgccattcaatcacgaaaggtaacgaaaaatgctctcatggttcaaatagtccaatggcccatagtggcctaaaaggcagccagttgacacctggccatgctaagcttccttataccatccgctaatgactttccattgggcccacaatttacggattcataattttaaaagaggagaaggccaagttaggttcattccccttattctgtcaataaaacaaatcaaactcatgtctatctaactgctcagggaggagcctttgcatgagaaaattctcatattctaagactgagtcatagaaatgagggtattacttttcttactgcaattaacctaaacaaaagccatattttaacaaatagatatttgcatggtacccttcatatattccaagcatttcactcattattccaggtaggttaagagcttctgaagtgtatgaagtaaaggtcagcaatcctttggggtgaacagtggcctcctttggagtttgggggtaacctgagacttcccaccaatgtccacctccatctgtgtacctaattcctattacctagttatggctcctctaggatcatttccaaacactctggatgtccaggaaatttaaattgtagcttttgactgagctagtttttcctatttatattaataaattttcaaaaatgcttgaaatcttcacatttgcaacaactttagttttcatgcacatacaaacacagagagacaaaaattccaaacagacactctccaaaagccaccacactctttcacttgctctatagtcatttagccaaccagccatgcagagaatatttaaaacttaaagattgagacattattctcagtttttgctgaggctttgtaacgaaattgaacactataagcagctattgtagtaattttggttaaaattgtttgcctgggatatagtatttgaggcagaagcacgtgtgtgaaggaggtgaggtggtttggaaagagtgaagactcgcagccagattgaatgtctggataattactataattctcccttcttggttgaaaccatgttctctcttgatttttaaacccaggctgcctctggaaacaagcaaacctgagtctttctaacctgagtcttcccaatcattagatttcttttctgtcctaacgatgaatgataaaaggacttgatgttcacaatttggggttataaggcaggtctgaaatctggagactcaagatgctggaaggagtggaaagtttcgatgactttatatgaatcactttgcactctatgtttggcttgtcctctttgaaactgatttactaaaataaatgtaagggaactattactccaaaagattaacttggcaggaaataccaatactttcagtttatgaaagacaaaactgtcttgttgctacaggaagctgcaatgttcctaacctttaaggttggtgttgaatagggtggtcatgccctcccctgcaggtatctttaggctcctgttgacctcctggtactataactgttcgtcttctctgggtagctattgattttgaactttaacatgcttcaaaactttattcatcagggaaataggaaaagagttttgttacctggaggaaatctattgtgatctacctgagctttttaaaaacagaccaggagaaggaaaccagtaatttttaaagaagagacagagaatgggataatagtttcacccaggatctctttctaaccctttcccttcaaatgaacttattggaacagaattggaaagaagaaaggacatctctgcccaccccacaggatgccaaaaaggctaaagaattacctctgtagatttaaacatctttaatggcttatgtatagatttgctaatacagagagaaatgaactattaaataaaaatcacattttataatatttttatggcttaaaacatcctttatctcctttttgttctctctacatgatatggtaagtgatgaggaaaatttaggctcaggaaggttaaaatctttcttggagttacacatctaagagagctgcagagctgacacttgtacccaggttttctgactgcaaatccagtttctttctattgcgttcttcccctttccctgcctcaagcagaaacaggttttttattttcaacctttatgtatacagtatgttatgttacatctacagctaagtttctttttagaagaatgtgagcccttctagctttggtttagagtgattctagaagccaatttccttggcttagtgattctatgcacctttcctaaacttagctttctaaggaaatgaagtgtacgagtgagaatgaattcacaatttcgacatgtaggtagcatcctaaagtgaaaagaggaggaaatttgtggtcaaagcactctccccaccacttagaaacttactgactgtgggcagcttcctcctccaagtttccttcctgatttacaagaccgtggtgtggtcaggattaaacttgaatacatgtaaggaagcctgaaagtgtctaacacatagcgagtattcaaatgccaccttctatttgatccttcccctccagttccttaagttttggaatctaggtttctcagttccaaatggattgacatttgcatatccccattgcacaatggatcaaataaactttatgttatcatttctccaacatagtgccagtaagcaaatcctttttaataacaacagtatgttgagaaacatatcaccaaataatatttaactttgtagctttgataagttctttaggttttggttttggttttgttttctgagacagggtcttgatctgtcacccagactggagtgcaatggttcaattttagctcactgcaacctgtaactcctgagctcaagtgatcctcccacctcagcctctcaagtagctgggactacaggtgtgctccaccatgcccagcgatttttttttttttttttttttttttttttggtagggacaaggtctcgctatgttgcccaggctggtcttcaattcctggccttaagtgatcctcctgcctcggcctcccaaagtgctggaattacaggcatgagccaccacccataactttatgtttgtttttttgatgcagtataagttcagcttgcttcttatgcagccataccatttcatgttaactctgatttttagcagcttattacattagtgttttattattaatataattttacagaaatttactaaaccatgactctgtagagttttaataatactacctccaaacatcattgcaaacatctagaagaatgaacaaaaatgatcttagatcgacagtatatctgtttgtcttagtttctacacaggatgttcagacatattccatttctttaaaaaaaaaatatatatatatatatatatatataggcctggcacggtggctcatgactgtaatcccagcactttgggaggcttaggcaggcaaatcacctgaggtcaggagtttgagactggcctgaccaacatggtgaaacctcgtctctattaaaattacaaaaattagccgggcgtggtggcacatgcctgtaatcccagctactcgggaggctgaggcaggagaatcacttgaacctgggaggcagaggttgcagtgagttgagatcacgccattgcactccagccttggtgacaagagtgaaactccgtctcaaaaaaaaaaaaaatatgtatatatatatatatatatatatatatatatatatatatatatatataaaatcccaccaaaagtctgcagagtgaccaaattagacggctctggtttcagattaaattctaaatgtgagaaaccacatagctcccatgatccatccaataattcctcacgtcctcttcactctttactccatgcataaaacagaatttttttttctcatcctgggtatgaagcaattaataatttacggatttagcctatttggattcaatcccttcaaactccatactacatccaaggtggaagtgacttaaactctgatatcaatcatcaggcttgtaatataggctttgttaatggcaggagagtctaataaaactttctgttccttatccttcatttaaatgaaaaactttttattgaaaacaatcataactctagctcatcataaatataattcatgaggacattttattatttttatattaaagaaataatattatagatgtaaactttgcacctttctaattattatcatgagttaagctaatacttgtcttctggtccctagatgatgattcttttttgccttactggaggagcccttgtcttgaagtgagttgcttcaacagcagaggacttctagtttctcccagttgagcctaaagtgaacttttcatcttcttcagaggaaggggcttccttgatttgtacttttgtggctcctcagataacacagacaattttatcttggatcccaggttctcttcaccattaagaataagaaagagagaaaatgctgtgcatgacagccacctactccaaactacccaaccccctgtaaccaggtaccttccaacaacgagatgattctgccctcactcaagagtctcccccacaaagattccattctccctttactttttattttttatttttttgcaaaacaaaggcctcctttagtacctccttagtttataacccttatcttcccagctcttcccttcactgatacctctgatttcaaaagttctgaagtcggaagaccacacaatttcagactgtgaacagaaattcagtcagaaattattggagttagaaagaatttagagaaattgtattgaactagaaagtcctctttgattagtggtggccctttagaaagttctagggcagagtcccatggtgtttcatcttttccatgatttgcttgaccaaaaacttttctttcacaacatgaaaatatgctaatccaccacacattttggattctgctctgtttgcctgaggtgttagatctctagccaggactgtgaagggaaggaacttgaatccttcctattgagctattaatgcagagtcagtgagatgaagggttccactcggggtcaaaatcatgtcagttaccaagcaaaggagcaagtaaggggaaacatctcctcatctggttagtggagccacatttcacccactgatcaagccagacctgagcaatagtctagatttctccctccacatctaattggtgacaatggtgattgtaccactgacaggtcacacaagtccacaccctccttcacagcctcactgcttcggctcttgtttatgctcttatcagcatctgtcactaggatccatttgtctcctgactcatctacttcctttcactcccctgggccatccttcacatcatgctagaagactgtgtctaacttgcagaacttattgtgtcaatctcctggttacggcccctccatggctccccacctgacataggaggcccttcacaatctggcttctgtcactcataacttgtctccagccttcattcttcagtctgattttatggttttctagttccccaatacaccacaccagtgtttatgaaaccctagtcattggcctacgaactttatgattattgacatattcatgtaccacctgtattattttttgcatagtgtttatttttaattgactacatttttaactacaataaagtaatttcaactaaaa

SEQ ID NO: 15 - DNA, 189 bp, Homo sapiens
ctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctaca

SEQ ID NO: 16 - DNA, 11482 bp, Homo sapiens
ccttgcctccccgattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgagatcatcactggtatgccagcctcaactgaaggagcatatgtgtcttcagagtctcccattagaatatcagtatccacagaaggagcaaatacttcttcatctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacaagcatcttgggattgaatttatggaggcggaggagctgtaccagaagagagtgctgaccataaccggcatctgcatcgccctccttgtggtcggcatcatgtgtgtggtggcctactgcaaaaccaagaaacagcggaaaaagctgcatgaccgtcttcggcagagccttcggtctgaacgaaacaatatgatgaacattgccaatgggcctcaccatcctaacccaccccccgagaatgtccagctggtgaatcaatacgtatctaaaaacgtcatctccagtgagcatattgttgagagagaagcagagacatccttttccaccagtcactatacttccacagcccatcactccactactgtcacccagactcctagccacagctggagcaacggacacactgaaagcatcctttccgaaagccactctgtaatcgtgatgtcatccgtagaaaacagtaggcacagcagcccaactgggggcccaagaggacgtcttaatggcacaggaggccctcgtgaatgtaacagcttcctcaggcatgccagagaaacccctgattcctaccgagactctcctcatagtgaaaggtatgtgtcagccatgaccaccccggctcgtatgtcacctgtagatttccacacgccaagctcccccaaatcgcccccttcggaaatgtctccacccgtgtccagcatgacggtgtccatgccttccatggcggtcagccccttcatggaagaagagagacctctacttctcgtgacaccaccaaggctgcgggagaagaagtttgaccatcaccctcagcagttcagctccttccaccacaaccccgcgcatgacagtaacagcctccctgctagccccttgaggatagtggaggatgaggagtatgaaacgacccaagagtacgagccagcccaagagcctgttaagaaactcgccaatagccggcgggccaaaagaaccaagcccaatggccacattgctaacagattggaagtggacagcaacacaagctcccagagcagtaactcagagagtgaaacagaagatgaaagagtaggtgaagatacgcctttcctgggcatacagaaccccctggcagccagtcttgaggcaacacctgccttccgcctggctgacagcaggactaacccagcaggccgcttctcgacacaggaagaaatccaggccaggctgtctagtgtaattgctaaccaagaccctattgctgtataaaacctaaataaacacatagattcacctgtaaaactttattttatataataaagtattccaccttaaattaaacaatttattttattttagcagttctgcaaatagaaaacaggaaaaaaacttttataaattaaatatatgtatgtaaaaatgtgttatgtgccatatgtagcaattttttacagtatttcaaaacgagaaagatatcaatggtgcctttatgttatgttatgtcgagagcaagttttgtacagttacagtgattgcttttccacagtatttctgcaaaacctctcatagattcagtttttgctggcttcttgtgcattgcattatgatgttgactggatgtatgatttgcaagacttgcaactgtccctctgtttgcttgtagtagcacccgatcagtatgtcttgtaatggcacatccatccagatatgcctctcttgtgtatgaagttttctttgctttcagaatatgaaatgagttgtgtctactctgccagccaaaggtttgcctcattgggctctgagataatagtagatccaacagcatgctactattaaatacagcaagaaactgcattaagtaatgttaaatattaggaagaaagtaatactgtgatttaaaaaaaactatattattaatcagaagacagcttgctcttactaaaaggagctctcatttactttatttgattttatttttcttgacaaaaagcaacagttttagggatagcttagaaaatgggttctggcttgctatcagggtaaatctaacaccttacaagaggactgagtgtcactttctctctgggggaatgatccagcagcttatctagttgacaatcaaaacacggctgataaaggtgcaatcatttctgacatgtatttttcactgattttgaagctagtgattggttgtgtcttcttggctcaaaaagaagcatattacggcacaaaaagcccagcccagacagcacatgcagcattttgtctgaaatacttctagagtcaaacgtgcctgctgtacatagcgatgacttgtcatcatagggaagtatttccatcgtagagtgttcagaaggagtgactgtataggtggagagaagcttagtgactccgttgaaattttaaaatgtggatgaccacccctttctcccccttatttttcttttatctttccatgttgccttgatcaggtcataactatgcatgaacattttttatcaggaatggccgatgtgtatgtgatttgtaatcacaagtaatgattcatcaggaaatgtcaatcctgttggaaagattgcacctttacttgcagaagtgacccccacctgtgtcctgacctctccatttacaggctctctcacccatttcccccacctcctttaatttttgctttactgtcataaagtaggactaagattggtctaagcattgcatgttcttttgtgatggtaaatccaaaggaaggcctataagtattaacatttgaaataactgctaattcaggaaaatggaagaaaaaaaattatttgaaacacagaacccatttcatggcctgcctgatatctgtgaaatcagggctggagctttacttaggattcacatggcctcctaggaaccatgggacaaatgggaaacaggttatcgggggattcatgaagtcagtgagagtaattgcttcttttttgcgggtgaactgaatgtatttcttcaccaaatcttgatgttaacaattaaaaagaagaaatgacatgcaagtaggtcttagcagaaaaatgcaggctgggcatgagtcatgttgttaccctcccacatgctcctacaatccacagagatgcctgtctgcaggttcttgaagttattgttagtatttggtatctcaaatttttcgtcactgttcacatgccactttctctgtgcacagtggtatcctcatttgctttttaacctacactgaggagtctttgtcaggttgcactgattttccaattctgcagtaatgagtaagctcacggcatggggaagaagacagtcagtccaatgaagttctctaaattattttaacattgcctttgaaggccttgactcatccttagctatttcaatgaagaaattcctaccatgaatttaaaaccctaaaaattctgtttcaaattctttgggcattggggtactcagatatcccattgtggaagaattttaagaataaatagaagtttctgttgagaaccatgagcaacatgtttcttacaatgagaattgctatgcattttaaaattgcaaatatatatgaaaattgaagacaagaggaaattgtatttctaacttgattctgatcactcacagaggtggcatattattatagttgggacatcctttgcacccttcataaaaaaggccagctgactgctcagcatcacctgccaaggccactagatttgtgtttacaggggtatctctgtgatgcttgtcacatcactcttgaccacctctgttaataaattccgacagtgcagtggcgatcggagtgtgaacttatgttcccagcatatggaaagctatcttaggttttaaggtagtagaaattgcccaggagtttgacagcaactttgtttcccgggtctaaaatcgtatcccactgaggtgtatgcagtggagcataatacatgcaaatacatgcaaaactccttttgtttcacctaagattcactttctatcttactttcccttcctgcctagtgtgacttttgcccccaagagtgcctggacagcattctagtttctacaaaatggtcctctgtgtaggtgaatgtgtcccaaacctgctatcactttcttgtttcagtgtgactgtcttgttagaggtgaagtttatccagggtaacttgctcactaactattcctttttatggcctggggttaaagggcgcatggctcacactggtgaaaataaggaaggcctggtcttatcttgtattaataatactggctgcattccaccagccagagatttctatctgcgaagacctatgaaacactgaagagaaatgtaggcagaaggaaatggccacatatcacaagttctattatatattcttttgtaaatacatattgtatattacttggatgttttcttatatcatttactgtctttttgagttaatgtcagtttttactctctcaacttactatgtaacattgtaaataacataatgtcctttattatttatatttaagcatctaacatatagagttgttttcatataagtttaagataaatgtcaaaaatatatgttcttttgtttttctttgctttaaaattatgtatcttttccttttcttttttttaagaataatttattgttcaggagaaagaatgtatatgtaactgaaactatctgaagaatgcacattgaaggccgtgaggtactgataaactaaagaatttattattcaaaatactaagcaataagtaattgtgatttatttaaagttttgtccattttccatgaaagacatactgcaataaaaatgctactctgtggagacctgggagtgttgctcagcagactacagcttcagtctgttagaccagcacctttcatctcattcccatagttatgctaatttaggattgtgttccatggaccccatgatcaccttgtctatacgttgcttcttgtctgtccattgcttttgccacaccacctgttctcaaatcatctcctccctaccaatgctgtttatcactttcttccttgttgaagaggccacacaaccagacagtactatgcttcctttttcctccatacacaataacagagagagaatattctagggcatgactgcctggatcctggctgttgctatcttttgtagtggcagtaagaaactccttcagactaatgaaaatgtcaacgtgccattcaatcacgaaaggtaacgaaaaatgctctcatggttcaaatagtccaatggcccatagtggcctaaaaggcagccagttgacacctggccatgctaagcttccttataccatccgctaatgactttccattgggcccacaatttacggattcataattttaaaagaggagaaggccaagttaggttcattccccttattctgtcaataaaacaaatcaaactcatgtctatctaactgctcagggaggagcctttgcatgagaaaattctcatattctaagactgagtcatagaaatgagggtattacttttcttactgcaattaacctaaacaaaagccatattttaacaaatagatatttgcatggtacccttcatatattccaagcatttcactcattattccaggtaggttaagagcttctgaagtgtatgaagtaaaggtcagcaatcctttggggtgaacagtggcctcctttggagtttgggggtaacctgagacttcccaccaatgtccacctccatctgtgtacctaattcctattacctagttatggctcctctaggatcatttccaaacactctggatgtccaggaaatttaaattgtagcttttgactgagctagtttttcctatttatattaataaattttcaaaaatgcttgaaatcttcacatttgcaacaactttagttttcatgcacatacaaacacagagagacaaaaattccaaacagacactctccaaaagccaccacactctttcacttgctctatagtcatttagccaaccagccatgcagagaatatttaaaacttaaagattgagacattattctcagtttttgctgaggctttgtaacgaaattgaacactataagcagctattgtagtaattttggttaaaattgtttgcctgggatatagtatttgaggcagaagcacgtgtgtgaaggaggtgaggtggtttggaaagagtgaagactcgcagccagattgaatgtctggataattactataattctcccttcttggttgaaaccatgttctctcttgatttttaaacccaggctgcctctggaaacaagcaaacctgagtctttctaacctgagtcttcccaatcattagatttcttttctgtcctaacgatgaatgataaaaggacttgatgttcacaatttggggttataaggcaggtctgaaatctggagactcaagatgctggaaggagtggaaagtttcgatgactttatatgaatcactttgcactctatgtttggcttgtcctctttgaaactgatttactaaaataaatgtaagggaactattactccaaaagattaacttggcaggaaataccaatactttcagtttatgaaagacaaaactgtcttgttgctacaggaagctgcaatgttcctaacctttaaggttggtgttgaatagggtggtcatgccctcccctgcaggtatctttaggctcctgttgacctcctggtactataactgttcgtcttctctgggtagctattgattttgaactttaacatgcttcaaaactttattcatcagggaaataggaaaagagttttgttacctggaggaaatctattgtgatctacctgagctttttaaaaacagaccaggagaaggaaaccagtaatttttaaagaagagacagagaatgggataatagtttcacccaggatctctttctaaccctttcccttcaaatgaacttattggaacagaattggaaagaagaaaggacatctctgcccaccccacaggatgccaaaaaggctaaagaattacctctgtagatttaaacatctttaatggcttatgtatagatttgctaatacagagagaaatgaactattaaataaaaatcacattttataatatttttatggcttaaaacatcctttatctcctttttgttctctctacatgatatggtaagtgatgaggaaaatttaggctcaggaaggttaaaatctttcttggagttacacatctaagagagctgcagagctgacacttgtacccaggttttctgactgcaaatccagtttctttctattgcgttcttcccctttccctgcctcaagcagaaacaggttttttattttcaacctttatgtatacagtatgttatgttacatctacagctaagtttctttttagaagaatgtgagcccttctagctttggtttagagtgattctagaagccaatttccttggcttagtgattctatgcacctttcctaaacttagctttctaaggaaatgaagtgtacgagtgagaatgaattcacaatttcgacatgtaggtagcatcctaaagtgaaaagaggaggaaatttgtggtcaaagcactctccccaccacttagaaacttactgactgtgggcagcttcctcctccaagtttccttcctgatttacaagaccgtggtgtggtcaggattaaacttgaatacatgtaaggaagcctgaaagtgtctaacacatagcgagtattcaaatgccaccttctatttgatccttcccctccagttccttaagttttggaatctaggtttctcagttccaaatggattgacatttgcatatccccattgcacaatggatcaaataaactttatgttatcatttctccaacatagtgccagtaagcaaatcctttttaataacaacagtatgttgagaaacatatcaccaaataatatttaactttgtagctttgataagttctttaggttttggttttggttttgttttctgagacagggtcttgatctgtcacccagactggagtgcaatggttcaattttagctcactgcaacctgtaactcctgagctcaagtgatcctcccacctcagcctctcaagtagctgggactacaggtgtgctccaccatgcccagcgatttttttttttttttttttttttttttttggtagggacaaggtctcgctatgttgcccaggctggtcttcaattcctggccttaagtgatcctcctgcctcggcctcccaaagtgctggaattacaggcatgagccaccacccataactttatgtttgtttttttgatgcagtataagttcagcttgcttcttatgcagccataccatttcatgttaactctgatttttagcagcttattacattagtgttttattattaatataattttacagaaatttactaaaccatgactctgtagagttttaataatactacctccaaacatcattgcaaacatctagaagaatgaacaaaaatgatcttagatcgacagtatatctgtttgtcttagtttctacacaggatgttcagacatattccatttctttaaaaaaaaaatatatatatatatatatatatataggcctggcacggtggctcatgactgtaatcccagcactttgggaggcttaggcaggcaaatcacctgaggtcaggagtttgagactggcctgaccaacatggtgaaacctcgtctctattaaaattacaaaaattagccgggcgtggtggcacatgcctgtaatcccagctactcgggaggctgaggcaggagaatcacttgaacctgggaggcagaggttgcagtgagttgagatcacgccattgcactccagccttggtgacaagagtgaaactccgtctcaaaaaaaaaaaaaatatgtatatatatatatatatatatatatatatatatatatatatatatataaaatcccaccaaaagtctgcagagtgaccaaattagacggctctggtttcagattaaattctaaatgtgagaaaccacatagctcccatgatccatccaataattcctcacgtcctcttcactctttactccatgcataaaacagaatttttttttctcatcctgggtatgaagcaattaataatttacggatttagcctatttggattcaatcccttcaaactccatactacatccaaggtggaagtgacttaaactctgatatcaatcatcaggcttgtaatataggctttgttaatggcaggagagtctaataaaactttctgttccttatccttcatttaaatgaaaaactttttattgaaaacaatcataactctagctcatcataaatataattcatgaggacattttattatttttatattaaagaaataatattatagatgtaaactttgcacctttctaattattatcatgagttaagctaatacttgtcttctggtccctagatgatgattcttttttgccttactggaggagcccttgtcttgaagtgagttgcttcaacagcagaggacttctagtttctcccagttgagcctaaagtgaacttttcatcttcttcagaggaaggggcttccttgatttgtacttttgtggctcctcagataacacagacaattttatcttggatcccaggttctcttcaccattaagaataagaaagagagaaaatgctgtgcatgacagccacctactccaaactacccaaccccctgtaaccaggtaccttccaacaacgagatgattctgccctcactcaagagtctcccccacaaagattccattctccctttactttttattttttatttttttgcaaaacaaaggcctcctttagtacctccttagtttataacccttatcttcccagctcttcccttcactgatacctctgatttcaaaagttctgaagtcggaagaccacacaatttcagactgtgaacagaaattcagtcagaaattattggagttagaaagaatttagagaaattgtattgaactagaaagtcctctttgattagtggtggccctttagaaagttctagggcagagtcccatggtgtttcatcttttccatgatttgcttgaccaaaaacttttctttcacaacatgaaaatatgctaatccaccacacattttggattctgctctgtttgcctgaggtgttagatctctagccaggactgtgaagggaaggaacttgaatccttcctattgagctattaatgcagagtcagtgagatgaagggttccactcggggtcaaaatcatgtcagttaccaagcaaaggagcaagtaaggggaaacatctcctcatctggttagtggagccacatttcacccactgatcaagccagacctgagcaatagtctagatttctccctccacatctaattggtgacaatggtgattgtaccactgacaggtcacacaagtccacaccctccttcacagcctcactgcttcggctcttgtttatgctcttatcagcatctgtcactaggatccatttgtctcctgactcatctacttcctttcactcccctgggccatccttcacatcatgctagaagactgtgtctaacttgcagaacttattgtgtcaatctcctggttacggcccctccatggctccccacctgacataggaggcccttcacaatctggcttctgtcactcataacttgtctccagccttcattcttcagtctgattttatggttttctagttccccaatacaccacaccagtgtttatgaaaccctagtcattggcctacgaactttatgattattgacatattcatgtaccacctgtattattttttgcatagtgtttatttttaattgactacatttttaactacaataaagtaatttcaactaaaa

SEQ ID NO: 17 - DNA, 11080 bp, Homo sapiens
ctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacaagcatcttgggattgaatttatggaggcggaggagctgtaccagaagagagtgctgaccataaccggcatctgcatcgccctccttgtggtcggcatcatgtgtgtggtggcctactgcaaaaccaagaaacagcggaaaaagctgcatgaccgtcttcggcagagccttcggtctgaacgaaacaatatgatgaacattgccaatgggcctcaccatcctaacccaccccccgagaatgtccagctggtgaatcaatacgtatctaaaaacgtcatctccagtgagcatattgttgagagagaagcagagacatccttttccaccagtcactatacttccacagcccatcactccactactgtcacccagactcctagccacagctggagcaacggacacactgaaagcatcctttccgaaagccactctgtaatcgtgatgtcatccgtagaaaacagtaggcacagcagcccaactgggggcccaagaggacgtcttaatggcacaggaggccctcgtgaatgtaacagcttcctcaggcatgccagagaaacccctgattcctaccgagactctcctcatagtgaaaggtatgtgtcagccatgaccaccccggctcgtatgtcacctgtagatttccacacgccaagctcccccaaatcgcccccttcggaaatgtctccacccgtgtccagcatgacggtgtccatgccttccatggcggtcagccccttcatggaagaagagagacctctacttctcgtgacaccaccaaggctgcgggagaagaagtttgaccatcaccctcagcagttcagctccttccaccacaaccccgcgcatgacagtaacagcctccctgctagccccttgaggatagtggaggatgaggagtatgaaacgacccaagagtacgagccagcccaagagcctgttaagaaactcgccaatagccggcgggccaaaagaaccaagcccaatggccacattgctaacagattggaagtggacagcaacacaagctcccagagcagtaactcagagagtgaaacagaagatgaaagagtaggtgaagatacgcctttcctgggcatacagaaccccctggcagccagtcttgaggcaacacctgccttccgcctggctgacagcaggactaacccagcaggccgcttctcgacacaggaagaaatccaggccaggctgtctagtgtaattgctaaccaagaccctattgctgtataaaacctaaataaacacatagattcacctgtaaaactttattttatataataaagtattccaccttaaattaaacaatttattttattttagcagttctgcaaatagaaaacaggaaaaaaacttttataaattaaatatatgtatgtaaaaatgtgttatgtgccatatgtagcaattttttacagtatttcaaaacgagaaagatatcaatggtgcctttatgttatgttatgtcgagagcaagttttgtacagttacagtgattgcttttccacagtatttctgcaaaacctctcatagattcagtttttgctggcttcttgtgcattgcattatgatgttgactggatgtatgatttgcaagacttgcaactgtccctctgtttgcttgtagtagcacccgatcagtatgtcttgtaatggcacatccatccagatatgcctctcttgtgtatgaagttttctttgctttcagaatatgaaatgagttgtgtctactctgccagccaaaggtttgcctcattgggctctgagataatagtagatccaacagcatgctactattaaatacagcaagaaactgcattaagtaatgttaaatattaggaagaaagtaatactgtgatttaaaaaaaactatattattaatcagaagacagcttgctcttactaaaaggagctctcatttactttatttgattttatttttcttgacaaaaagcaacagttttagggatagcttagaaaatgggttctggcttgctatcagggtaaatctaacaccttacaagaggactgagtgtcactttctctctgggggaatgatccagcagcttatctagttgacaatcaaaacacggctgataaaggtgcaatcatttctgacatgtatttttcactgattttgaagctagtgattggttgtgtcttcttggctcaaaaagaagcatattacggcacaaaaagcccagcccagacagcacatgcagcattttgtctgaaatacttctagagtcaaacgtgcctgctgtacatagcgatgacttgtcatcatagggaagtatttccatcgtagagtgttcagaaggagtgactgtataggtggagagaagcttagtgactccgttgaaattttaaaatgtggatgaccacccctttctcccccttatttttcttttatctttccatgttgccttgatcaggtcataactatgcatgaacattttttatcaggaatggccgatgtgtatgtgatttgtaatcacaagtaatgattcatcaggaaatgtcaatcctgttggaaagattgcacctttacttgcagaagtgacccccacctgtgtcctgacctctccatttacaggctctctcacccatttcccccacctcctttaatttttgctttactgtcataaagtaggactaagattggtctaagcattgcatgttcttttgtgatggtaaatccaaaggaaggcctataagtattaacatttgaaataactgctaattcaggaaaatggaagaaaaaaaattatttgaaacacagaacccatttcatggcctgcctgatatctgtgaaatcagggctggagctttacttaggattcacatggcctcctaggaaccatgggacaaatgggaaacaggttatcgggggattcatgaagtcagtgagagtaattgcttcttttttgcgggtgaactgaatgtatttcttcaccaaatcttgatgttaacaattaaaaagaagaaatgacatgcaagtaggtcttagcagaaaaatgcaggctgggcatgagtcatgttgttaccctcccacatgctcctacaatccacagagatgcctgtctgcaggttcttgaagttattgttagtatttggtatctcaaatttttcgtcactgttcacatgccactttctctgtgcacagtggtatcctcatttgctttttaacctacactgaggagtctttgtcaggttgcactgattttccaattctgcagtaatgagtaagctcacggcatggggaagaagacagtcagtccaatgaagttctctaaattattttaacattgcctttgaaggccttgactcatccttagctatttcaatgaagaaattcctaccatgaatttaaaaccctaaaaattctgtttcaaattctttgggcattggggtactcagatatcccattgtggaagaattttaagaataaatagaagtttctgttgagaaccatgagcaacatgtttcttacaatgagaattgctatgcattttaaaattgcaaatatatatgaaaattgaagacaagaggaaattgtatttctaacttgattctgatcactcacagaggtggcatattattatagttgggacatcctttgcacccttcataaaaaaggccagctgactgctcagcatcacctgccaaggccactagatttgtgtttacaggggtatctctgtgatgcttgtcacatcactcttgaccacctctgttaataaattccgacagtgcagtggcgatcggagtgtgaacttatgttcccagcatatggaaagctatcttaggttttaaggtagtagaaattgcccaggagtttgacagcaactttgtttcccgggtctaaaatcgtatcccactgaggtgtatgcagtggagcataatacatgcaaatacatgcaaaactccttttgtttcacctaagattcactttctatcttactttcccttcctgcctagtgtgacttttgcccccaagagtgcctggacagcattctagtttctacaaaatggtcctctgtgtaggtgaatgtgtcccaaacctgctatcactttcttgtttcagtgtgactgtcttgttagaggtgaagtttatccagggtaacttgctcactaactattcctttttatggcctggggttaaagggcgcatggctcacactggtgaaaataaggaaggcctggtcttatcttgtattaataatactggctgcattccaccagccagagatttctatctgcgaagacctatgaaacactgaagagaaatgtaggcagaaggaaatggccacatatcacaagttctattatatattcttttgtaaatacatattgtatattacttggatgttttcttatatcatttactgtctttttgagttaatgtcagtttttactctctcaacttactatgtaacattgtaaataacataatgtcctttattatttatatttaagcatctaacatatagagttgttttcatataagtttaagataaatgtcaaaaatatatgttcttttgtttttctttgctttaaaattatgtatcttttccttttcttttttttaagaataatttattgttcaggagaaagaatgtatatgtaactgaaactatctgaagaatgcacattgaaggccgtgaggtactgataaactaaagaatttattattcaaaatactaagcaataagtaattgtgatttatttaaagttttgtccattttccatgaaagacatactgcaataaaaatgctactctgtggagacctgggagtgttgctcagcagactacagcttcagtctgttagaccagcacctttcatctcattcccatagttatgctaatttaggattgtgttccatggaccccatgatcaccttgtctatacgttgcttcttgtctgtccattgcttttgccacaccacctgttctcaaatcatctcctccctaccaatgctgtttatcactttcttccttgttgaagaggccacacaaccagacagtactatgcttcctttttcctccatacacaataacagagagagaatattctagggcatgactgcctggatcctggctgttgctatcttttgtagtggcagtaagaaactccttcagactaatgaaaatgtcaacgtgccattcaatcacgaaaggtaacgaaaaatgctctcatggttcaaatagtccaatggcccatagtggcctaaaaggcagccagttgacacctggccatgctaagcttccttataccatccgctaatgactttccattgggcccacaatttacggattcataattttaaaagaggagaaggccaagttaggttcattccccttattctgtcaataaaacaaatcaaactcatgtctatctaactgctcagggaggagcctttgcatgagaaaattctcatattctaagactgagtcatagaaatgagggtattacttttcttactgcaattaacctaaacaaaagccatattttaacaaatagatatttgcatggtacccttcatatattccaagcatttcactcattattccaggtaggttaagagcttctgaagtgtatgaagtaaaggtcagcaatcctttggggtgaacagtggcctcctttggagtttgggggtaacctgagacttcccaccaatgtccacctccatctgtgtacctaattcctattacctagttatggctcctctaggatcatttccaaacactctggatgtccaggaaatttaaattgtagcttttgactgagctagtttttcctatttatattaataaattttcaaaaatgcttgaaatcttcacatttgcaacaactttagttttcatgcacatacaaacacagagagacaaaaattccaaacagacactctccaaaagccaccacactctttcacttgctctatagtcatttagccaaccagccatgcagagaatatttaaaacttaaagattgagacattattctcagtttttgctgaggctttgtaacgaaattgaacactataagcagctattgtagtaattttggttaaaattgtttgcctgggatatagtatttgaggcagaagcacgtgtgtgaaggaggtgaggtggtttggaaagagtgaagactcgcagccagattgaatgtctggataattactataattctcccttcttggttgaaaccatgttctctcttgatttttaaacccaggctgcctctggaaacaagcaaacctgagtctttctaacctgagtcttcccaatcattagatttcttttctgtcctaacgatgaatgataaaaggacttgatgttcacaatttggggttataaggcaggtctgaaatctggagactcaagatgctggaaggagtggaaagtttcgatgactttatatgaatcactttgcactctatgtttggcttgtcctctttgaaactgatttactaaaataaatgtaagggaactattactccaaaagattaacttggcaggaaataccaatactttcagtttatgaaagacaaaactgtcttgttgctacaggaagctgcaatgttcctaacctttaaggttggtgttgaatagggtggtcatgccctcccctgcaggtatctttaggctcctgttgacctcctggtactataactgttcgtcttctctgggtagctattgattttgaactttaacatgcttcaaaactttattcatcagggaaataggaaaagagttttgttacctggaggaaatctattgtgatctacctgagctttttaaaaacagaccaggagaaggaaaccagtaatttttaaagaagagacagagaatgggataatagtttcacccaggatctctttctaaccctttcccttcaaatgaacttattggaacagaattggaaagaagaaaggacatctctgcccaccccacaggatgccaaaaaggctaaagaattacctctgtagatttaaacatctttaatggcttatgtatagatttgctaatacagagagaaatgaactattaaataaaaatcacattttataatatttttatggcttaaaacatcctttatctcctttttgttctctctacatgatatggtaagtgatgaggaaaatttaggctcaggaaggttaaaatctttcttggagttacacatctaagagagctgcagagctgacacttgtacccaggttttctgactgcaaatccagtttctttctattgcgttcttcccctttccctgcctcaagcagaaacaggttttttattttcaacctttatgtatacagtatgttatgttacatctacagctaagtttctttttagaagaatgtgagcccttctagctttggtttagagtgattctagaagccaatttccttggcttagtgattctatgcacctttcctaaacttagctttctaaggaaatgaagtgtacgagtgagaatgaattcacaatttcgacatgtaggtagcatcctaaagtgaaaagaggaggaaatttgtggtcaaagcactctccccaccacttagaaacttactgactgtgggcagcttcctcctccaagtttccttcctgatttacaagaccgtggtgtggtcaggattaaacttgaatacatgtaaggaagcctgaaagtgtctaacacatagcgagtattcaaatgccaccttctatttgatccttcccctccagttccttaagttttggaatctaggtttctcagttccaaatggattgacatttgcatatccccattgcacaatggatcaaataaactttatgttatcatttctccaacatagtgccagtaagcaaatcctttttaataacaacagtatgttgagaaacatatcaccaaataatatttaactttgtagctttgataagttctttaggttttggttttggttttgttttctgagacagggtcttgatctgtcacccagactggagtgcaatggttcaattttagctcactgcaacctgtaactcctgagctcaagtgatcctcccacctcagcctctcaagtagctgggactacaggtgtgctccaccatgcccagcgatttttttttttttttttttttttttttttggtagggacaaggtctcgctatgttgcccaggctggtcttcaattcctggccttaagtgatcctcctgcctcggcctcccaaagtgctggaattacaggcatgagccaccacccataactttatgtttgtttttttgatgcagtataagttcagcttgcttcttatgcagccataccatttcatgttaactctgatttttagcagcttattacattagtgttttattattaatataattttacagaaatttactaaaccatgactctgtagagttttaataatactacctccaaacatcattgcaaacatctagaagaatgaacaaaaatgatcttagatcgacagtatatctgtttgtcttagtttctacacaggatgttcagacatattccatttctttaaaaaaaaaatatatatatatatatatatatataggcctggcacggtggctcatgactgtaatcccagcactttgggaggcttaggcaggcaaatcacctgaggtcaggagtttgagactggcctgaccaacatggtgaaacctcgtctctattaaaattacaaaaattagccgggcgtggtggcacatgcctgtaatcccagctactcgggaggctgaggcaggagaatcacttgaacctgggaggcagaggttgcagtgagttgagatcacgccattgcactccagccttggtgacaagagtgaaactccgtctcaaaaaaaaaaaaaatatgtatatatatatatatatatatatatatatatatatatatatatatataaaatcccaccaaaagtctgcagagtgaccaaattagacggctctggtttcagattaaattctaaatgtgagaaaccacatagctcccatgatccatccaataattcctcacgtcctcttcactctttactccatgcataaaacagaatttttttttctcatcctgggtatgaagcaattaataatttacggatttagcctatttggattcaatcccttcaaactccatactacatccaaggtggaagtgacttaaactctgatatcaatcatcaggcttgtaatataggctttgttaatggcaggagagtctaataaaactttctgttccttatccttcatttaaatgaaaaactttttattgaaaacaatcataactctagctcatcataaatataattcatgaggacattttattatttttatattaaagaaataatattatagatgtaaactttgcacctttctaattattatcatgagttaagctaatacttgtcttctggtccctagatgatgattcttttttgccttactggaggagcccttgtcttgaagtgagttgcttcaacagcagaggacttctagtttctcccagttgagcctaaagtgaacttttcatcttcttcagaggaaggggcttccttgatttgtacttttgtggctcctcagataacacagacaattttatcttggatcccaggttctcttcaccattaagaataagaaagagagaaaatgctgtgcatgacagccacctactccaaactacccaaccccctgtaaccaggtaccttccaacaacgagatgattctgccctcactcaagagtctcccccacaaagattccattctccctttactttttattttttatttttttgcaaaacaaaggcctcctttagtacctccttagtttataacccttatcttcccagctcttcccttcactgatacctctgatttcaaaagttctgaagtcggaagaccacacaatttcagactgtgaacagaaattcagtcagaaattattggagttagaaagaatttagagaaattgtattgaactagaaagtcctctttgattagtggtggccctttagaaagttctagggcagagtcccatggtgtttcatcttttccatgatttgcttgaccaaaaacttttctttcacaacatgaaaatatgctaatccaccacacattttggattctgctctgtttgcctgaggtgttagatctctagccaggactgtgaagggaaggaacttgaatccttcctattgagctattaatgcagagtcagtgagatgaagggttccactcggggtcaaaatcatgtcagttaccaagcaaaggagcaagtaaggggaaacatctcctcatctggttagtggagccacatttcacccactgatcaagccagacctgagcaatagtctagatttctccctccacatctaattggtgacaatggtgattgtaccactgacaggtcacacaagtccacaccctccttcacagcctcactgcttcggctcttgtttatgctcttatcagcatctgtcactaggatccatttgtctcctgactcatctacttcctttcactcccctgggccatccttcacatcatgctagaagactgtgtctaacttgcagaacttattgtgtcaatctcctggttacggcccctccatggctccccacctgacataggaggcccttcacaatctggcttctgtcactcataacttgtctccagccttcattcttcagtctgattttatggttttctagttccccaatacaccacaccagtgtttatgaaaccctagtcattggcctacgaactttatgattattgacatattcatgtaccacctgtattattttttgcatagtgtttatttttaattgactacatttttaactacaataaagtaatttcaactaaaa

SEQ ID NO: 18 - DNA, 11083 bp, Homo sapiens
catctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacaagcatcttgggattgaatttatggaggcggaggagctgtaccagaagagagtgctgaccataaccggcatctgcatcgccctccttgtggtcggcatcatgtgtgtggtggcctactgcaaaaccaagaaacagcggaaaaagctgcatgaccgtcttcggcagagccttcggtctgaacgaaacaatatgatgaacattgccaatgggcctcaccatcctaacccaccccccgagaatgtccagctggtgaatcaatacgtatctaaaaacgtcatctccagtgagcatattgttgagagagaagcagagacatccttttccaccagtcactatacttccacagcccatcactccactactgtcacccagactcctagccacagctggagcaacggacacactgaaagcatcctttccgaaagccactctgtaatcgtgatgtcatccgtagaaaacagtaggcacagcagcccaactgggggcccaagaggacgtcttaatggcacaggaggccctcgtgaatgtaacagcttcctcaggcatgccagagaaacccctgattcctaccgagactctcctcatagtgaaaggtatgtgtcagccatgaccaccccggctcgtatgtcacctgtagatttccacacgccaagctcccccaaatcgcccccttcggaaatgtctccacccgtgtccagcatgacggtgtccatgccttccatggcggtcagccccttcatggaagaagagagacctctacttctcgtgacaccaccaaggctgcgggagaagaagtttgaccatcaccctcagcagttcagctccttccaccacaaccccgcgcatgacagtaacagcctccctgctagccccttgaggatagtggaggatgaggagtatgaaacgacccaagagtacgagccagcccaagagcctgttaagaaactcgccaatagccggcgggccaaaagaaccaagcccaatggccacattgctaacagattggaagtggacagcaacacaagctcccagagcagtaactcagagagtgaaacagaagatgaaagagtaggtgaagatacgcctttcctgggcatacagaaccccctggcagccagtcttgaggcaacacctgccttccgcctggctgacagcaggactaacccagcaggccgcttctcgacacaggaagaaatccaggccaggctgtctagtgtaattgctaaccaagaccctattgctgtataaaacctaaataaacacatagattcacctgtaaaactttattttatataataaagtattccaccttaaattaaacaatttattttattttagcagttctgcaaatagaaaacaggaaaaaaacttttataaattaaatatatgtatgtaaaaatgtgttatgtgccatatgtagcaattttttacagtatttcaaaacgagaaagatatcaatggtgcctttatgttatgttatgtcgagagcaagttttgtacagttacagtgattgcttttccacagtatttctgcaaaacctctcatagattcagtttttgctggcttcttgtgcattgcattatgatgttgactggatgtatgatttgcaagacttgcaactgtccctctgtttgcttgtagtagcacccgatcagtatgtcttgtaatggcacatccatccagatatgcctctcttgtgtatgaagttttctttgctttcagaatatgaaatgagttgtgtctactctgccagccaaaggtttgcctcattgggctctgagataatagtagatccaacagcatgctactattaaatacagcaagaaactgcattaagtaatgttaaatattaggaagaaagtaatactgtgatttaaaaaaaactatattattaatcagaagacagcttgctcttactaaaaggagctctcatttactttatttgattttatttttcttgacaaaaagcaacagttttagggatagcttagaaaatgggttctggcttgctatcagggtaaatctaacaccttacaagaggactgagtgtcactttctctctgggggaatgatccagcagcttatctagttgacaatcaaaacacggctgataaaggtgcaatcatttctgacatgtatttttcactgattttgaagctagtgattggttgtgtcttcttggctcaaaaagaagcatattacggcacaaaaagcccagcccagacagcacatgcagcattttgtctgaaatacttctagagtcaaacgtgcctgctgtacatagcgatgacttgtcatcatagggaagtatttccatcgtagagtgttcagaaggagtgactgtataggtggagagaagcttagtgactccgttgaaattttaaaatgtggatgaccacccctttctcccccttatttttcttttatctttccatgttgccttgatcaggtcataactatgcatgaacattttttatcaggaatggccgatgtgtatgtgatttgtaatcacaagtaatgattcatcaggaaatgtcaatcctgttggaaagattgcacctttacttgcagaagtgacccccacctgtgtcctgacctctccatttacaggctctctcacccatttcccccacctcctttaatttttgctttactgtcataaagtaggactaagattggtctaagcattgcatgttcttttgtgatggtaaatccaaaggaaggcctataagtattaacatttgaaataactgctaattcaggaaaatggaagaaaaaaaattatttgaaacacagaacccatttcatggcctgcctgatatctgtgaaatcagggctggagctttacttaggattcacatggcctcctaggaaccatgggacaaatgggaaacaggttatcgggggattcatgaagtcagtgagagtaattgcttcttttttgcgggtgaactgaatgtatttcttcaccaaatcttgatgttaacaattaaaaagaagaaatgacatgcaagtaggtcttagcagaaaaatgcaggctgggcatgagtcatgttgttaccctcccacatgctcctacaatccacagagatgcctgtctgcaggttcttgaagttattgttagtatttggtatctcaaatttttcgtcactgttcacatgccactttctctgtgcacagtggtatcctcatttgctttttaacctacactgaggagtctttgtcaggttgcactgattttccaattctgcagtaatgagtaagctcacggcatggggaagaagacagtcagtccaatgaagttctctaaattattttaacattgcctttgaaggccttgactcatccttagctatttcaatgaagaaattcctaccatgaatttaaaaccctaaaaattctgtttcaaattctttgggcattggggtactcagatatcccattgtggaagaattttaagaataaatagaagtttctgttgagaaccatgagcaacatgtttcttacaatgagaattgctatgcattttaaaattgcaaatatatatgaaaattgaagacaagaggaaattgtatttctaacttgattctgatcactcacagaggtggcatattattatagttgggacatcctttgcacccttcataaaaaaggccagctgactgctcagcatcacctgccaaggccactagatttgtgtttacaggggtatctctgtgatgcttgtcacatcactcttgaccacctctgttaataaattccgacagtgcagtggcgatcggagtgtgaacttatgttcccagcatatggaaagctatcttaggttttaaggtagtagaaattgcccaggagtttgacagcaactttgtttcccgggtctaaaatcgtatcccactgaggtgtatgcagtggagcataatacatgcaaatacatgcaaaactccttttgtttcacctaagattcactttctatcttactttcccttcctgcctagtgtgacttttgcccccaagagtgcctggacagcattctagtttctacaaaatggtcctctgtgtaggtgaatgtgtcccaaacctgctatcactttcttgtttcagtgtgactgtcttgttagaggtgaagtttatccagggtaacttgctcactaactattcctttttatggcctggggttaaagggcgcatggctcacactggtgaaaataaggaaggcctggtcttatcttgtattaataatactggctgcattccaccagccagagatttctatctgcgaagacctatgaaacactgaagagaaatgtaggcagaaggaaatggccacatatcacaagttctattatatattcttttgtaaatacatattgtatattacttggatgttttcttatatcatttactgtctttttgagttaatgtcagtttttactctctcaacttactatgtaacattgtaaataacataatgtcctttattatttatatttaagcatctaacatatagagttgttttcatataagtttaagataaatgtcaaaaatatatgttcttttgtttttctttgctttaaaattatgtatcttttccttttcttttttttaagaataatttattgttcaggagaaagaatgtatatgtaactgaaactatctgaagaatgcacattgaaggccgtgaggtactgataaactaaagaatttattattcaaaatactaagcaataagtaattgtgatttatttaaagttttgtccattttccatgaaagacatactgcaataaaaatgctactctgtggagacctgggagtgttgctcagcagactacagcttcagtctgttagaccagcacctttcatctcattcccatagttatgctaatttaggattgtgttccatggaccccatgatcaccttgtctatacgttgcttcttgtctgtccattgcttttgccacaccacctgttctcaaatcatctcctccctaccaatgctgtttatcactttcttccttgttgaagaggccacacaaccagacagtactatgcttcctttttcctccatacacaataacagagagagaatattctagggcatgactgcctggatcctggctgttgctatcttttgtagtggcagtaagaaactccttcagactaatgaaaatgtcaacgtgccattcaatcacgaaaggtaacgaaaaatgctctcatggttcaaatagtccaatggcccatagtggcctaaaaggcagccagttgacacctggccatgctaagcttccttataccatccgctaatgactttccattgggcccacaatttacggattcataattttaaaagaggagaaggccaagttaggttcattccccttattctgtcaataaaacaaatcaaactcatgtctatctaactgctcagggaggagcctttgcatgagaaaattctcatattctaagactgagtcatagaaatgagggtattacttttcttactgcaattaacctaaacaaaagccatattttaacaaatagatatttgcatggtacccttcatatattccaagcatttcactcattattccaggtaggttaagagcttctgaagtgtatgaagtaaaggtcagcaatcctttggggtgaacagtggcctcctttggagtttgggggtaacctgagacttcccaccaatgtccacctccatctgtgtacctaattcctattacctagttatggctcctctaggatcatttccaaacactctggatgtccaggaaatttaaattgtagcttttgactgagctagtttttcctatttatattaataaattttcaaaaatgcttgaaatcttcacatttgcaacaactttagttttcatgcacatacaaacacagagagacaaaaattccaaacagacactctccaaaagccaccacactctttcacttgctctatagtcatttagccaaccagccatgcagagaatatttaaaacttaaagattgagacattattctcagtttttgctgaggctttgtaacgaaattgaacactataagcagctattgtagtaattttggttaaaattgtttgcctgggatatagtatttgaggcagaagcacgtgtgtgaaggaggtgaggtggtttggaaagagtgaagactcgcagccagattgaatgtctggataattactataattctcccttcttggttgaaaccatgttctctcttgatttttaaacccaggctgcctctggaaacaagcaaacctgagtctttctaacctgagtcttcccaatcattagatttcttttctgtcctaacgatgaatgataaaaggacttgatgttcacaatttggggttataaggcaggtctgaaatctggagactcaagatgctggaaggagtggaaagtttcgatgactttatatgaatcactttgcactctatgtttggcttgtcctctttgaaactgatttactaaaataaatgtaagggaactattactccaaaagattaacttggcaggaaataccaatactttcagtttatgaaagacaaaactgtcttgttgctacaggaagctgcaatgttcctaacctttaaggttggtgttgaatagggtggtcatgccctcccctgcaggtatctttaggctcctgttgacctcctggtactataactgttcgtcttctctgggtagctattgattttgaactttaacatgcttcaaaactttattcatcagggaaataggaaaagagttttgttacctggaggaaatctattgtgatctacctgagctttttaaaaacagaccaggagaaggaaaccagtaatttttaaagaagagacagagaatgggataatagtttcacccaggatctctttctaaccctttcccttcaaatgaacttattggaacagaattggaaagaagaaaggacatctctgcccaccccacaggatgccaaaaaggctaaagaattacctctgtagatttaaacatctttaatggcttatgtatagatttgctaatacagagagaaatgaactattaaataaaaatcacattttataatatttttatggcttaaaacatcctttatctcctttttgttctctctacatgatatggtaagtgatgaggaaaatttaggctcaggaaggttaaaatctttcttggagttacacatctaagagagctgcagagctgacacttgtacccaggttttctgactgcaaatccagtttctttctattgcgttcttcccctttccctgcctcaagcagaaacaggttttttattttcaacctttatgtatacagtatgttatgttacatctacagctaagtttctttttagaagaatgtgagcccttctagctttggtttagagtgattctagaagccaatttccttggcttagtgattctatgcacctttcctaaacttagctttctaaggaaatgaagtgtacgagtgagaatgaattcacaatttcgacatgtaggtagcatcctaaagtgaaaagaggaggaaatttgtggtcaaagcactctccccaccacttagaaacttactgactgtgggcagcttcctcctccaagtttccttcctgatttacaagaccgtggtgtggtcaggattaaacttgaatacatgtaaggaagcctgaaagtgtctaacacatagcgagtattcaaatgccaccttctatttgatccttcccctccagttccttaagttttggaatctaggtttctcagttccaaatggattgacatttgcatatccccattgcacaatggatcaaataaactttatgttatcatttctccaacatagtgccagtaagcaaatcctttttaataacaacagtatgttgagaaacatatcaccaaataatatttaactttgtagctttgataagttctttaggttttggttttggttttgttttctgagacagggtcttgatctgtcacccagactggagtgcaatggttcaattttagctcactgcaacctgtaactcctgagctcaagtgatcctcccacctcagcctctcaagtagctgggactacaggtgtgctccaccatgcccagcgatttttttttttttttttttttttttttttggtagggacaaggtctcgctatgttgcccaggctggtcttcaattcctggccttaagtgatcctcctgcctcggcctcccaaagtgctggaattacaggcatgagccaccacccataactttatgtttgtttttttgatgcagtataagttcagcttgcttcttatgcagccataccatttcatgttaactctgatttttagcagcttattacattagtgttttattattaatataattttacagaaatttactaaaccatgactctgtagagttttaataatactacctccaaacatcattgcaaacatctagaagaatgaacaaaaatgatcttagatcgacagtatatctgtttgtcttagtttctacacaggatgttcagacatattccatttctttaaaaaaaaaatatatatatatatatatatatataggcctggcacggtggctcatgactgtaatcccagcactttgggaggcttaggcaggcaaatcacctgaggtcaggagtttgagactggcctgaccaacatggtgaaacctcgtctctattaaaattacaaaaattagccgggcgtggtggcacatgcctgtaatcccagctactcgggaggctgaggcaggagaatcacttgaacctgggaggcagaggttgcagtgagttgagatcacgccattgcactccagccttggtgacaagagtgaaactccgtctcaaaaaaaaaaaaaatatgtatatatatatatatatatatatatatatatatatatatatatatataaaatcccaccaaaagtctgcagagtgaccaaattagacggctctggtttcagattaaattctaaatgtgagaaaccacatagctcccatgatccatccaataattcctcacgtcctcttcactctttactccatgcataaaacagaatttttttttctcatcctgggtatgaagcaattaataatttacggatttagcctatttggattcaatcccttcaaactccatactacatccaaggtggaagtgacttaaactctgatatcaatcatcaggcttgtaatataggctttgttaatggcaggagagtctaataaaactttctgttccttatccttcatttaaatgaaaaactttttattgaaaacaatcataactctagctcatcataaatataattcatgaggacattttattatttttatattaaagaaataatattatagatgtaaactttgcacctttctaattattatcatgagttaagctaatacttgtcttctggtccctagatgatgattcttttttgccttactggaggagcccttgtcttgaagtgagttgcttcaacagcagaggacttctagtttctcccagttgagcctaaagtgaacttttcatcttcttcagaggaaggggcttccttgatttgtacttttgtggctcctcagataacacagacaattttatcttggatcccaggttctcttcaccattaagaataagaaagagagaaaatgctgtgcatgacagccacctactccaaactacccaaccccctgtaaccaggtaccttccaacaacgagatgattctgccctcactcaagagtctcccccacaaagattccattctccctttactttttattttttatttttttgcaaaacaaaggcctcctttagtacctccttagtttataacccttatcttcccagctcttcccttcactgatacctctgatttcaaaagttctgaagtcggaagaccacacaatttcagactgtgaacagaaattcagtcagaaattattggagttagaaagaatttagagaaattgtattgaactagaaagtcctctttgattagtggtggccctttagaaagttctagggcagagtcccatggtgtttcatcttttccatgatttgcttgaccaaaaacttttctttcacaacatgaaaatatgctaatccaccacacattttggattctgctctgtttgcctgaggtgttagatctctagccaggactgtgaagggaaggaacttgaatccttcctattgagctattaatgcagagtcagtgagatgaagggttccactcggggtcaaaatcatgtcagttaccaagcaaaggagcaagtaaggggaaacatctcctcatctggttagtggagccacatttcacccactgatcaagccagacctgagcaatagtctagatttctccctccacatctaattggtgacaatggtgattgtaccactgacaggtcacacaagtccacaccctccttcacagcctcactgcttcggctcttgtttatgctcttatcagcatctgtcactaggatccatttgtctcctgactcatctacttcctttcactcccctgggccatccttcacatcatgctagaagactgtgtctaacttgcagaacttattgtgtcaatctcctggttacggcccctccatggctccccacctgacataggaggcccttcacaatctggcttctgtcactcataacttgtctccagccttcattcttcagtctgattttatggttttctagttccccaatacaccacaccagtgtttatgaaaccctagtcattggcctacgaactttatgattattgacatattcatgtaccacctgtattattttttgcatagtgtttatttttaattgactacatttttaactacaataaagtaatttcaactaaaa

SEQ ID NO: 19 - DNA, 94 bp, Homo sapiens
caggtcctgagcctcgagccgcagcacgagctcaaattccgagccttgcctccccgattgaaagagatgaaaagccaggaatcggctgcaggtt

SEQ ID NO: 20 - DNA, 399 bp, Homo sapiens
agtccccgcccctggagccggcggcgcagggcgcagcttcccgccgccagagcgggccagcctgctgcgtgcgtgcgtgtgtacgactctgcgtgcgtgcgtgcgtgcgtgcgtgccgtcagctcgccgggcaccgcggcctcgccctcgccctccgcccctgcgcctgcaccgcgtagaccgacccccccccagcgcgcccacccggtagaggacccccgcccgtgccccgaccggtccccgcctttttgtaaaacttaaagcgggcgcagcattaacgcttcccgccccggtgacctctcaggggtctccccgccaaaggtgctccgccgctaaggaacatggcgaaggtggagcaggtcctgagcctcgagccgcagcacgagctcaaattccgag

SEQ ID NO: 21 - DNA, 153 bp, Homo sapiens
gtcccttcaccgatgttgtcaccaccaacctaaagcttggcaacccgacagaccgaaatgtgtgttttaaggtgaagactacagcaccacgtaggtactgtgtgaggcccaacagcggaatcatcgatgcaggggcctcaattaatgtatctg

SEQ ID NO: 22 - DNA, 104 bp, Homo sapiens
tgatgttacagcctttcgattatgatcccaatgagaaaagtaaacacaagtttatggttcagtctatgtttgctccaactgacacttcagatatggaagcagta

SEQ ID NO: 23 - DNA, 81 bp, Homo sapiens
tggaaggaggcaaaaccggaagaccttatggattcaaaacttagatgtgtgtttgaattgccagcagagaatgataaacca

SEQ ID NO: 24 - DNA, 177 bp, Homo sapiens
catgatgtagaaataaataaaattatatccacaactgcatcaaagacagaaacaccaatagtgtctaagtctctgagttcttctttggatgacaccgaagttaagaaggttatggaagaatgtaagaggctgcaaggtgaagttcagaggctacgggaggagaacaagcagttcaag

SEQ ID NO: 25 - DNA, 7035 bp, Homo sapiens
gaagaagatggactgcggatgaggaagacagtgcagagcaacagccccatttcagcattagccccaactgggaaggaagaaggccttagcacccggctcttggctctggtggttttgttctttatcgttggtgtaattattgggaagattgccttgtagaggtagcatgcacaggatggtaaattggattggtggatccaccatatcatgggatttaaatttatcataaccatgtgtaaaaagaaattaatgtatgatgacatctcacaggtcttgcctttaaattacccctccctgcacacacatacacagatacacacacacaaatataatgtaacgatcttttagaaagttaaaaatgtatagtaactgattgagggggaaaagaatgatctttattaatgacaagggaaaccatgagtaatgccacaatggcatattgtaaatgtcattttaaacattggtaggccttggtacatgatgctggattacctctcttaaaatgacacccttcctcgcctgttggtgctggcccttggggagctggagcccagcatgctggggagtgcggtcagctccacacagtagtccccacgtggcccactcccggcccaggctgctttccgtgtcttcagttctgtccaagccatcagctccttgggactgatgaacagagtcagaagcccaaaggaattgcactgtggcagcatcagacgtactcgtcataagtgagaggcgtgtgttgactgattgacccagcgctttggaaataaatggcagtgctttgttcacttaaagggaccaagctaaatttgtattggttcatgtagtgaagtcaaactgttattcagagatgtttaatgcatatttaacttatttaatgtatttcatctcatgttttcttattgtcacaagagtacagttaatgctgcgtgctgctgaactctgttgggtgaactggtattgctgctggagggctgtgggctcctctgtctctggagagtctggtcatgtggaggtggggtttattgggatgctggagaagagctgccaggaagtgttttttctgggtcagtaaataacaactgtcatagggagggaaattctcagtagtgacagtcaactctaggttaccttttttaatgaagagtagtcagtcttctagattgttcttataccacctctcaaccattactcacacttccagcgcccaggtccaagtctgagcctgacctccccttggggacctagcctggagtcaggacaaatggatcgggctgcagagggttagaagcgagggcaccagcagttgtgggtggggagcaagggaagagagaaactcttcagcgaatccttctagtactagttgagagtttgactgtgaattaattttatgccataaaagaccaacccagttctgtttgactatgtagcatcttgaaaagaaaaattataataaagccccaaaattaagaattcttttgtcattttgtcacatttgctctatggggggaattattattttatcatttttattattttgccattggaaggttaactttaaaatgagccctatcactgagaaatacgtgtttcatgatttaactctgtgtgtgtgtgtgtgtgtgtgtgtgtgtgtgtgtgtattttttttttggttgtcttcagctgacagtatgaaaaatgaaactgctgaaaaagctgagcacctggtcacccttggccttccattgctttggccttcagtaaaaagcagcctcccttctaggtcagggaaccatgccattgagactagtaacgggcgttctgggcacagtcccactgtgcacaggtttgagaggacaagttcatcagaaggaaggcagtccttagaagtcacatacgttgagccacgttgctcctaagcctggctctgtcaagctgggtcaggggccttgaaactggagaagtggaagtctatggttggtctgagtaagtaacttcctgtcttcatgaaaaaagttgactttgaatcccaggtactcacagaaatggtgaacagacttagttgttacccaggcacccatggattgtgttgagtgtgcagacagggaggccaccccaataggaattcgtctccaggatttttcccatgtgtcccccagtacttataaaagggagtgaaaagaccgagctgtaaggcatgtgccttctgccacctgactttccgtgaggggactaaaatttactaattgtagttgctgcagccagttaagtcctgtagcttccaggccctcatgtctttgataggagagtgcttaggtggtccccaacagtgcctaggggtacagtacagtcccattacactagagcagggctctatttatttttaaaggatatggccgtgtgttttgataaaactttattcacaaaaacagccgggtcatgggatttggcttgtgagtctgtaacagttcttaaaaagaatatctgagaaactactctgttttagacctttgaaggtgatttagagttttgtgtacatctaggagaaggtgttcagcttctcagaggatgtggacattttggttgcagctaaaaatcagtctctgaagtctctctcccttctagaggttaggacttggtgaacatgtttgtgggccttttgactgagtggcagaaggaaactgctcaggaagagaaacaggtgactgatgggaaggttgattattttctcagtcatcctggcagccaaaaatgtgccaggaaaagaaagaatgtggagcacgcgtggctcctggaggacttggagatgcatgcacatttagggtgttttccctagaattacataatgaaaaaaagaataaggcaaagaggaggtgaatatggggcctgtcacaacggcctgccctgccccaagagggttaagagtcagataatcgggacgaaactggcatggaaagagcgagcctagggaggatgccgctgggcagtgtgcatgggggagctgctgccaggctgccctccagtctgctcctgtggttactggctccacagcacctcagagagggcggccctggcttcagaaatgccagccatagtgctcacaaatgcagaagagatggaagcggtgacagaatcctgaaagtttttattgattgaagttttaaattggtaacttaagcttccttggcacgatacaaaatacctcttaaagacagcaggcttttttatttgtaggtgtgaggaactggctttaacttttttctcctcctagtttgcatgttttccttctctcgtcttctgaactgctggcaccagcagtaatacatactgataaaatcaaaattgatttttaccagtggccagtttatggctagagagacgacttatacctccataacacagaagggggaaaaatgaagaacctccagtgatccgtgaaaacctaaacgctttcaaacaaatcccaggaacagaattgctatcgaaagatatcattgcccagtttgcaggctatgttgagtcagatagaactgaatgtagtgagagctcagagctacagagcctttcagatgaatttgaaaacagactctgtgtgtgtgtgcatgtgtgcatgtgtgcatgtgtggcatatgtgccgtatgtcagtagcttgacagttttcaaatcgtgcctatatttttttgcatacacaaatttttgtgtttgcaaactcagaatccatgccaaaatacaatgttatatgtcattttcagctccttctctaaaggaatggcccatttctcattgtagtttgagaaatacatgtatgaagagataggggtcttgggcttcccagtgtcactttgaacacctgaataacatttaactcctgagaccttctcggtgtagaggccactgcttccccctgctggagatggcatttcattgaagggcctctcgtggctttccctgcccccggctgtctggcctgaagaaggagaaagaaccaaactgaactatgaaaagttaccactctgaggagacctctcttaattaacacttggggccatgtttgctgttgttgagaaggagtgttctcaaagatgagctggaatggaattgtatttagaaaggcccctgcaaagtatatagatggatgactctagttcatgacatacaaatcccataaggccaacgaccactcttctggaacaccaagagcagctctgagatcatgctggccctacgcgaattgagtttctgtggcctaattggatttggagaacgccttccctggccccttttcctcagacagatctgctctgataggaaccttttcaagaaagttactgttgtttcaatgccactccttacctgtatagaacatttccaatacattcgctcattgaacttaatccttgcaactgtgactggggggtagatggctctgtttgcatacgaagaaataaaggctccaggaggttaaatcgggcaactttttagaactaaatcagtctctgtaaggcctacattgctaagataccatttcagctctgaaaatctgcttcagggaagtgagtggatgaggccttcctgcctcagctactctgcccgtctgtacatcttttgtgtctgcctccgtaccttattcagttattttcacactaaagtaagtagaattaagactgtagttcagatgctttttctttttctgttggaaactgaacacactacagacagtgaaaaaaggtacatattccattttctcattgcctgaagatctctgctgatgctcctggagaatgactttgggggctttagaaagaatattgccagtccgtctcggcaaggagatgatgggagcgctttatatggaggctttacatgacttgtaaattaaatgtgaatgagggcagttgattaaaattggtattacagaagggccctgctgaggtttgaaaacagctgagctgctgatgtctcaggcctttccctgaattagcactgcggttctccaggatatcagcaaagagggcaagtaatagaagcccctgataaggagcgtcagccgacaggcaagcttgggaggctgtgggaatgggtctgcccccagcttcacagacctcttcctccagcctctgaatcccattagccacagcctagaacattagctgagctgcacaagctcacccacccctgtgccagggggccctgacctccctccatgccatgtttttggctgtatctacggcacttaacaataggggctttttattttcattacagagatattttgaaaaatttaaaagacatgaactcacataaacagttatggatgatagttaaaagagaaacgggtggaggtggatgagaggttgtcttcatgaatataattacttgagatttttttttcttaatggaattagtttattagaaaatgtctgtgttaaatccgtagaaaaggaagaaaagtgtagcaacaaaaatgtagccattatctaacttgccataaatatttgcagttatgataccttggaatgttgccacgatatggattgctttgattaaaagatgtcagttgaataaaacagtactgtgggagaatcgctttctgctgctagataaatgctgatgtttatttttaaaccaggaaacattgatcctgtaacaatgcccgattacaattgctttattacaccccagggctgatggagatgtaatcacttggctaatggatgtgggtgcaggacagatgctcgcttgctggcctgctttcctgcttgcattctgatgagctgcaggagtgcgcctggccttctgcaggtggagctgctgtcagagcttcgtttcactgatacccaaagccatgtctgactgaaataaaacaggttccctttttttttccctttggaaaatgccaactaagggagactaatcagatatcttaacacaatttcatccaggcttagtgctaacaagattgcggggctttttagggtttaagaagatgagaaatgagtgtgcacgtttcacacgttgacttgccggtttttccatgtcatacaaaaaagtcctggctgtttctccgaactggctgcctgcattcccgtctttcttttgtttttaagaaatagactgaattcagctgttaatcctctagtacagtatccatgttaaaatgtttttccattgcatcttttatgtgaattcaaaggtcagaatttattgtctgtgatattgagaccatgtgtacaagaactactttttgcttttcatcattcactccttagcaaacgtttcgtaagtaccctctgtctgtttgctactatatgaggtgctgcgaaattagtgggcgtggctttttatatttttcattcgtgtgtagcctaagtaaggtgactcaagatgatacaccgagagaaaaatgcaaaatatatttggttctcatttctgttgctgtcgtttcctttttaaagacgatttatcaactgctgccatttggaacttcctataagaaactaaaaatgatctatttcagtgttcctttcgcctttcctctgctttctgaataaatggtttcagtaacccatgctgttctctccctattctacgtctttctccctatgttgaaaaaagattcccacagtttctgatgtgtgtgtttatagtcttcaatgtatgttaacatgttaggaactgagtatcttaagagatgtcttagaatgctttagttttcataatttgtcctttatgtatttttcattgtatttgctgttttgacatggaagtaatttaaaaagttggtgcaggaaaggactctttactgttgcacattttggttttctgatatgtaataaattcatggcttggcagctgacatgatgtttcccagagagaaggagatgtatttctgcagggtccagaccaaaagagccatttacagcatgttctcccatgttccattatcagcctgatgaaacctgccctgccaaggcataaacttttgtactagctgtctccatattatgttcaataaattctgtgctctgaatatatttaaaaaaaaaaaaa

SEQ ID NO: 26 - DNA, 7949 bp, Homo sapiens
agtccccgcccctggagccggcggcgcagggcgcagcttcccgccgccagagcgggccagcctgctgcgtgcgtgcgtgtgtacgactctgcgtgcgtgcgtgcgtgcgtgcgtgccgtcagctcgccgggcaccgcggcctcgccctcgccctccgcccctgcgcctgcaccgcgtagaccgacccccccccagcgcgcccacccggtagaggacccccgcccgtgccccgaccggtccccgcctttttgtaaaacttaaagcgggcgcagcattaacgcttcccgccccggtgacctctcaggggtctccccgccaaaggtgctccgccgctaaggaacatggcgaaggtggagcaggtcctgagcctcgagccgcagcacgagctcaaattccgaggtcccttcaccgatgttgtcaccaccaacctaaagcttggcaacccgacagaccgaaatgtgtgttttaaggtgaagactacagcaccacgtaggtactgtgtgaggcccaacagcggaatcatcgatgcaggggcctcaattaatgtatctgtgatgttacagcctttcgattatgatcccaatgagaaaagtaaacacaagtttatggttcagtctatgtttgctccaactgacacttcagatatggaagcagtatggaaggaggcaaaaccggaagaccttatggattcaaaacttagatgtgtgtttgaattgccagcagagaatgataaaccacatgatgtagaaataaataaaattatatccacaactgcatcaaagacagaaacaccaatagtgtctaagtctctgagttcttctttggatgacaccgaagttaagaaggttatggaagaatgtaagaggctgcaaggtgaagttcagaggctacgggaggagaacaagcagttcaaggaagaagatggactgcggatgaggaagacagtgcagagcaacagccccatttcagcattagccccaactgggaaggaagaaggccttagcacccggctcttggctctggtggttttgttctttatcgttggtgtaattattgggaagattgccttgtagaggtagcatgcacaggatggtaaattggattggtggatccaccatatcatgggatttaaatttatcataaccatgtgtaaaaagaaattaatgtatgatgacatctcacaggtcttgcctttaaattacccctccctgcacacacatacacagatacacacacacaaatataatgtaacgatcttttagaaagttaaaaatgtatagtaactgattgagggggaaaagaatgatctttattaatgacaagggaaaccatgagtaatgccacaatggcatattgtaaatgtcattttaaacattggtaggccttggtacatgatgctggattacctctcttaaaatgacacccttcctcgcctgttggtgctggcccttggggagctggagcccagcatgctggggagtgcggtcagctccacacagtagtccccacgtggcccactcccggcccaggctgctttccgtgtcttcagttctgtccaagccatcagctccttgggactgatgaacagagtcagaagcccaaaggaattgcactgtggcagcatcagacgtactcgtcataagtgagaggcgtgtgttgactgattgacccagcgctttggaaataaatggcagtgctttgttcacttaaagggaccaagctaaatttgtattggttcatgtagtgaagtcaaactgttattcagagatgtttaatgcatatttaacttatttaatgtatttcatctcatgttttcttattgtcacaagagtacagttaatgctgcgtgctgctgaactctgttgggtgaactggtattgctgctggagggctgtgggctcctctgtctctggagagtctggtcatgtggaggtggggtttattgggatgctggagaagagctgccaggaagtgttttttctgggtcagtaaataacaactgtcatagggagggaaattctcagtagtgacagtcaactctaggttaccttttttaatgaagagtagtcagtcttctagattgttcttataccacctctcaaccattactcacacttccagcgcccaggtccaagtctgagcctgacctccccttggggacctagcctggagtcaggacaaatggatcgggctgcagagggttagaagcgagggcaccagcagttgtgggtggggagcaagggaagagagaaactcttcagcgaatccttctagtactagttgagagtttgactgtgaattaattttatgccataaaagaccaacccagttctgtttgactatgtagcatcttgaaaagaaaaattataataaagccccaaaattaagaattcttttgtcattttgtcacatttgctctatggggggaattattattttatcatttttattattttgccattggaaggttaactttaaaatgagccctatcactgagaaatacgtgtttcatgatttaactctgtgtgtgtgtgtgtgtgtgtgtgtgtgtgtgtgtgtattttttttttggttgtcttcagctgacagtatgaaaaatgaaactgctgaaaaagctgagcacctggtcacccttggccttccattgctttggccttcagtaaaaagcagcctcccttctaggtcagggaaccatgccattgagactagtaacgggcgttctgggcacagtcccactgtgcacaggtttgagaggacaagttcatcagaaggaaggcagtccttagaagtcacatacgttgagccacgttgctcctaagcctggctctgtcaagctgggtcaggggccttgaaactggagaagtggaagtctatggttggtctgagtaagtaacttcctgtcttcatgaaaaaagttgactttgaatcccaggtactcacagaaatggtgaacagacttagttgttacccaggcacccatggattgtgttgagtgtgcagacagggaggccaccccaataggaattcgtctccaggatttttcccatgtgtcccccagtacttataaaagggagtgaaaagaccgagctgtaaggcatgtgccttctgccacctgactttccgtgaggggactaaaatttactaattgtagttgctgcagccagttaagtcctgtagcttccaggccctcatgtctttgataggagagtgcttaggtggtccccaacagtgcctaggggtacagtacagtcccattacactagagcagggctctatttatttttaaaggatatggccgtgtgttttgataaaactttattcacaaaaacagccgggtcatgggatttggcttgtgagtctgtaacagttcttaaaaagaatatctgagaaactactctgttttagacctttgaaggtgatttagagttttgtgtacatctaggagaaggtgttcagcttctcagaggatgtggacattttggttgcagctaaaaatcagtctctgaagtctctctcccttctagaggttaggacttggtgaacatgtttgtgggccttttgactgagtggcagaaggaaactgctcaggaagagaaacaggtgactgatgggaaggttgattattttctcagtcatcctggcagccaaaaatgtgccaggaaaagaaagaatgtggagcacgcgtggctcctggaggacttggagatgcatgcacatttagggtgttttccctagaattacataatgaaaaaaagaataaggcaaagaggaggtgaatatggggcctgtcacaacggcctgccctgccccaagagggttaagagtcagataatcgggacgaaactggcatggaaagagcgagcctagggaggatgccgctgggcagtgtgcatgggggagctgctgccaggctgccctccagtctgctcctgtggttactggctccacagcacctcagagagggcggccctggcttcagaaatgccagccatagtgctcacaaatgcagaagagatggaagcggtgacagaatcctgaaagtttttattgattgaagttttaaattggtaacttaagcttccttggcacgatacaaaatacctcttaaagacagcaggcttttttatttgtaggtgtgaggaactggctttaacttttttctcctcctagtttgcatgttttccttctctcgtcttctgaactgctggcaccagcagtaatacatactgataaaatcaaaattgatttttaccagtggccagtttatggctagagagacgacttatacctccataacacagaagggggaaaaatgaagaacctccagtgatccgtgaaaacctaaacgctttcaaacaaatcccaggaacagaattgctatcgaaagatatcattgcccagtttgcaggctatgttgagtcagatagaactgaatgtagtgagagctcagagctacagagcctttcagatgaatttgaaaacagactctgtgtgtgtgtgcatgtgtgcatgtgtgcatgtgtggcatatgtgccgtatgtcagtagcttgacagttttcaaatcgtgcctatatttttttgcatacacaaatttttgtgtttgcaaactcagaatccatgccaaaatacaatgttatatgtcattttcagctccttctctaaaggaatggcccatttctcattgtagtttgagaaatacatgtatgaagagataggggtcttgggcttcccagtgtcactttgaacacctgaataacatttaactcctgagaccttctcggtgtagaggccactgcttccccctgctggagatggcatttcattgaagggcctctcgtggctttccctgcccccggctgtctggcctgaagaaggagaaagaaccaaactgaactatgaaaagttaccactctgaggagacctctcttaattaacacttggggccatgtttgctgttgttgagaaggagtgttctcaaagatgagctggaatggaattgtatttagaaaggcccctgcaaagtatatagatggatgactctagttcatgacatacaaatcccataaggccaacgaccactcttctggaacaccaagagcagctctgagatcatgctggccctacgcgaattgagtttctgtggcctaattggatttggagaacgccttccctggccccttttcctcagacagatctgctctgataggaaccttttcaagaaagttactgttgtttcaatgccactccttacctgtatagaacatttccaatacattcgctcattgaacttaatccttgcaactgtgactggggggtagatggctctgtttgcatacgaagaaataaaggctccaggaggttaaatcgggcaactttttagaactaaatcagtctctgtaaggcctacattgctaagataccatttcagctctgaaaatctgcttcagggaagtgagtggatgaggccttcctgcctcagctactctgcccgtctgtacatcttttgtgtctgcctccgtaccttattcagttattttcacactaaagtaagtagaattaagactgtagttcagatgctttttctttttctgttggaaactgaacacactacagacagtgaaaaaaggtacatattccattttctcattgcctgaagatctctgctgatgctcctggagaatgactttgggggctttagaaagaatattgccagtccgtctcggcaaggagatgatgggagcgctttatatggaggctttacatgacttgtaaattaaatgtgaatgagggcagttgattaaaattggtattacagaagggccctgctgaggtttgaaaacagctgagctgctgatgtctcaggcctttccctgaattagcactgcggttctccaggatatcagcaaagagggcaagtaatagaagcccctgataaggagcgtcagccgacaggcaagcttgggaggctgtgggaatgggtctgcccccagcttcacagacctcttcctccagcctctgaatcccattagccacagcctagaacattagctgagctgcacaagctcacccacccctgtgccagggggccctgacctccctccatgccatgtttttggctgtatctacggcacttaacaataggggctttttattttcattacagagatattttgaaaaatttaaaagacatgaactcacataaacagttatggatgatagttaaaagagaaacgggtggaggtggatgagaggttgtcttcatgaatataattacttgagatttttttttcttaatggaattagtttattagaaaatgtctgtgttaaatccgtagaaaaggaagaaaagtgtagcaacaaaaatgtagccattatctaacttgccataaatatttgcagttatgataccttggaatgttgccacgatatggattgctttgattaaaagatgtcagttgaataaaacagtactgtgggagaatcgctttctgctgctagataaatgctgatgtttatttttaaaccaggaaacattgatcctgtaacaatgcccgattacaattgctttattacaccccagggctgatggagatgtaatcacttggctaatggatgtgggtgcaggacagatgctcgcttgctggcctgctttcctgcttgcattctgatgagctgcaggagtgcgcctggccttctgcaggtggagctgctgtcagagcttcgtttcactgatacccaaagccatgtctgactgaaataaaacaggttccctttttttttccctttggaaaatgccaactaagggagactaatcagatatcttaacacaatttcatccaggcttagtgctaacaagattgcggggctttttagggtttaagaagatgagaaatgagtgtgcacgtttcacacgttgacttgccggtttttccatgtcatacaaaaaagtcctggctgtttctccgaactggctgcctgcattcccgtctttcttttgtttttaagaaatagactgaattcagctgttaatcctctagtacagtatccatgttaaaatgtttttccattgcatcttttatgtgaattcaaaggtcagaatttattgtctgtgatattgagaccatgtgtacaagaactactttttgcttttcatcattcactccttagcaaacgtttcgtaagtaccctctgtctgtttgctactatatgaggtgctgcgaaattagtgggcgtggctttttatatttttcattcgtgtgtagcctaagtaaggtgactcaagatgatacaccgagagaaaaatgcaaaatatatttggttctcatttctgttgctgtcgtttcctttttaaagacgatttatcaactgctgccatttggaacttcctataagaaactaaaaatgatctatttcagtgttcctttcgcctttcctctgctttctgaataaatggtttcagtaacccatgctgttctctccctattctacgtctttctccctatgttgaaaaaagattcccacagtttctgatgtgtgtgtttatagtcttcaatgtatgttaacatgttaggaactgagtatcttaagagatgtcttagaatgctttagttttcataatttgtcctttatgtatttttcattgtatttgctgttttgacatggaagtaatttaaaaagttggtgcaggaaaggactctttactgttgcacattttggttttctgatatgtaataaattcatggcttggcagctgacatgatgtttcccagagagaaggagatgtatttctgcagggtccagaccaaaagagccatttacagcatgttctcccatgttccattatcagcctgatgaaacctgccctgccaaggcataaacttttgtactagctgtctccatattatgttcaataaattctgtgctctgaatatatttaaaaaaaaaaaaa

SEQ ID NO: 27 - DNA, 85 bp, Homo sapiens
agcttcaaacatagtggggaaagctcactcggattatatgctgtatgtatacgctacatctacatccaccactgggacaagccat

SEQ ID NO: 28 - DNA, 253 bp, Homo sapiens
ggttgggctcgcggcgctgtgattggtctgcccggactccgcctccagcgcatgtcattagcatctcattagctgtccgctcgggctccggaggcagccaacgccgccagtctgaggcaggtgcccgacatggcgagtgtagtgctgccgagcggatcccagtgtgcggcggcagcggcggcggcggcgcctcccgggctccggctccggcttctgctgttgctcttctccgccgcggcactgatccccacag

SEQ ID NO: 29 - DNA, 147 bp, Homo sapiens
gtgatgggcagaatctgtttacgaaagacgtgacagtgatcgagggagaggttgcgaccatcagttgccaagtcaataagagtgacgactctgtgattcagctactgaatcccaacaggcagaccatttatttcagggacttcaggc

SEQ ID NO: 30 - DNA, 153 bp, Homo sapiens
ctttgaaggacagcaggtttcagttgctgaatttttctagcagtgaactcaaagtatcattgacaaacgtctcaatttctgatgaaggaagatacttttgccagctctataccgatcccccacaggaaagttacaccaccatcacagtcctgg

SEQ ID NO: 31 - DNA, 138 bp, Homo sapiens
tcccaccacgtaatctgatgatcgatatccagaaagacactgcggtggaaggtgaggagattgaagtcaactgcactgctatggccagcaagccagccacgactatcaggtggttcaaagggaacacagagctaaaag

SEQ ID NO: 32 - DNA, 159 bp, Homo sapiens
gcaaatcggaggtggaagagtggtcagacatgtacactgtgaccagtcagctgatgctgaaggtgcacaaggaggacgatggggtcccagtgatctgccaggtggagcaccctgcggtcactggaaacctgcagacccagcggtatctagaagtacagt

SEQ ID NO: 33 - DNA, 100 bp, Homo sapiens
ataagcctcaagtgcacattcagatgacttatcctctacaaggcttaacccgggaaggggacgcgcttgagttaacatgtgaagccatcgggaagcccca

SEQ ID NO: 34 - DNA, 173 bp, Homo sapiens
gcctgtgatggtaacttgggtgagagtcgatgatgaaatgcctcaacacgccgtactgtctgggcccaacctgttcatcaataacctaaacaaaacagataatggtacataccgctgtgaagcttcaaacatagtggggaaagctcactcggattatatgctgtatgtatacg

SEQ ID NO: 35 - DNA, 33 bp, Homo sapiens
acacaacggcgacgacagaaccagcagttcacg

SEQ ID NO: 36 - DNA, 54 bp, Homo sapiens
gccttactcagttgcccaattccgcagaagaactggacagtgaggacctctcag

SEQ ID NO: 37 - DNA, 132 bp, Homo sapiens
attcccgagcaggtgaagaaggctcgatcagggcagtggatcatgccgtgatcggtggcgtcgtggcggtggtggtgttcgccatgctgtgcttgctcatcattctggggcgctattttgccagacataaag

SEQ ID NO: 38 - DNA, 2968 bp, Homo sapiens
gtacatacttcactcatgaagccaaaggagccgatgacgcagcagacgcagacacagctataatcaatgcagaaggaggacagaacaactccgaagaaaagaaagagtacttcatctagatcagcctttttgtttcaatgaggtgtccaactggccctatttagatgataaagagacagtgatattggaacttgcgagaaattcgtgtgtttttttatgaatgggtggaaaggtgtgagactgggaaggcttgggatttgctgtgtaaaaaaaaaaaaaatgttctttggaaagtacactctgctgtttgacacctcttttttcgtttgtttgtttgtttaatttttatttcttcctaccaagtcaaacttggatacttggatttagtttcagtagattgcagaaaattctgtgccttgttttttgtttgtttgttgcgttcctttcttttccccctttgtgcacatttatttcctccctctaccccaatttcggattttttccaaaatctcccattttggaatttgcctgctgggattccttagactcttttccttcccttttctgttctagttttttacttttgtttatttttatggtaactgctttctgttccaaattcagtttcataaaaggagaaccagcacagcttagatttcatagttcagaatttagtgtatccataatgcattcttctctgttgtcgtaaagatttgggtgaacaaacaatgaaaactctttgctgctgcccatgtttcaaatacttagagcagtgaagactagaaaattagactgtgattcagaaaatgttctgtttgctgtggaactacattactgtacagggttatctgcaagtgaggtgtgtcacaatgagattgaatttcactgtctttaattctgtatctgtagacggctcagtatagataccctacgctgtccagaaaggtttggggcagaaaggactcctcctttttccatgccctaaacagacctgacaggtgaggtctgttccttttatataagtggacaaattttgagttgccacaggaggggaagtagggaggggggaaatacagttctgctctggttgtttctgttccaaatgattccatccacctttcccaatcggccttacttctcactaatttgtaggaaaaagcaagttcgtctgttgtgcgaatgactgaatgggacagagttgatttttttttttttttcctttgtgcttagttaggaaggcagtaggatgtggcctgcatgtactgtatattacagatatttgtcatgctgggatttccaactcgaatctgtgtgaaactttcattccttcagatttggcttgacaaaggcaggaggtacaaaagaagggctggtattgttctcacactggtctgctgtcgctctcagttctcgataggtcagagcagaggtggaaaaacagcatgtacggattttcagttacttaatcaaaactcaaatgtgagtgtttttatctttttacctttcatacactagccttggcctctttcctcagccttaagaaccatctgccaaaaattactgatcctcgcatgatggcagccatagtgcatagctactaaaatcagtgaccttgaacatatcttagatggggagcctcgggaaaaggtagaggagtcacgttaccatttacatgttttaaagaaagaagtgtggggattttcactgaaacgtctaggaaatctagaagtagtcctgaaggacagaaactaaactcttaccatatgtttggtaagactccagactccagctaacagtccctatggaaagatggcatcaaaaaagatagatctatatatatatataaatatatattctattacattttcagtgagtaattttggattttgcaaggtgcatttttactattgttacattatgtggaaaacttatgctgatttatttaagggggaaaaagtgtcaactctttgttatttgaaaacatgtttatttttcttgtctttattttaacctttgatagaaccattgcaatatgggggccttttgggaacggactggtatgtaaaagaaaatccattatcgagcagcattttatttacccctcccctatccctaggcacttaaccaagacaaaaagccacaatgaacatccctttttcaatgaattttataatctgcagctctattccgagcccttagcacccattccgaccatagtataatcatatcaaagggtgagaatcatttagcatgttgttgaaaggttttttttcagttgttctttttagaaaaaaagaaaaacaaaaacaaaaacaaaaaaaaaaaatcacaccattgctcacagaattggcatctcatttttgggacctcccatctttctgttttgaaaagtgtacagtagtgcagtgttcctgatgtaactttatggcttacaatgttgacatgtctcaggttcatgtgttgcgattggtgttttccgtctcaggtagattgcaaagtgtaggccccacacattggaaaaaataataataaaacaaagcaaaaacaggaaattatggattttagttgtatattggtttatgtattttttcttaagtatacagtgcactgtttgaaatgtattgttgagtattactttgtacaggttgatcactttttttagagtgaagaaagaacaaacttgttttttgtgttttttaaaggaatataaaataatgaaggatgtataattgatgccaaataagcttgttctttagtcacaccgacgtcttatttttccctttaggccagttctgtttttaaggtgtacatggacaatgttacagtgtaagaaactccatatccatatgttcccattcgcattttgtattggttcatgtataccatttttacaaaaaaaaaaagaaaaaaaagaagtactataaaatatctgtcttcttaataaaaaaaaattaatgttacaaagtga

SEQ ID NO: 39 - DNA, 4310 bp, Homo sapiens
ggttgggctcgcggcgctgtgattggtctgcccggactccgcctccagcgcatgtcattagcatctcattagctgtccgctcgggctccggaggcagccaacgccgccagtctgaggcaggtgcccgacatggcgagtgtagtgctgccgagcggatcccagtgtgcggcggcagcggcggcggcggcgcctcccgggctccggctccggcttctgctgttgctcttctccgccgcggcactgatccccacaggtgatgggcagaatctgtttacgaaagacgtgacagtgatcgagggagaggttgcgaccatcagttgccaagtcaataagagtgacgactctgtgattcagctactgaatcccaacaggcagaccatttatttcagggacttcaggcctttgaaggacagcaggtttcagttgctgaatttttctagcagtgaactcaaagtatcattgacaaacgtctcaatttctgatgaaggaagatacttttgccagctctataccgatcccccacaggaaagttacaccaccatcacagtcctggtcccaccacgtaatctgatgatcgatatccagaaagacactgcggtggaaggtgaggagattgaagtcaactgcactgctatggccagcaagccagccacgactatcaggtggttcaaagggaacacagagctaaaaggcaaatcggaggtggaagagtggtcagacatgtacactgtgaccagtcagctgatgctgaaggtgcacaaggaggacgatggggtcccagtgatctgccaggtggagcaccctgcggtcactggaaacctgcagacccagcggtatctagaagtacagtataagcctcaagtgcacattcagatgacttatcctctacaaggcttaacccgggaaggggacgcgcttgagttaacatgtgaagccatcgggaagccccagcctgtgatggtaacttgggtgagagtcgatgatgaaatgcctcaacacgccgtactgtctgggcccaacctgttcatcaataacctaaacaaaacagataatggtacataccgctgtgaagcttcaaacatagtggggaaagctcactcggattatatgctgtatgtatacgacacaacggcgacgacagaaccagcagttcacggccttactcagttgcccaattccgcagaagaactggacagtgaggacctctcagattcccgagcaggtgaagaaggctcgatcagggcagtggatcatgccgtgatcggtggcgtcgtggcggtggtggtgttcgccatgctgtgcttgctcatcattctggggcgctattttgccagacataaaggtacatacttcactcatgaagccaaaggagccgatgacgcagcagacgcagacacagctataatcaatgcagaaggaggacagaacaactccgaagaaaagaaagagtacttcatctagatcagcctttttgtttcaatgaggtgtccaactggccctatttagatgataaagagacagtgatattggaacttgcgagaaattcgtgtgtttttttatgaatgggtggaaaggtgtgagactgggaaggcttgggatttgctgtgtaaaaaaaaaaaaaatgttctttggaaagtacactctgctgtttgacacctcttttttcgtttgtttgtttgtttaatttttatttcttcctaccaagtcaaacttggatacttggatttagtttcagtagattgcagaaaattctgtgccttgttttttgtttgtttgttgcgttcctttcttttccccctttgtgcacatttatttcctccctctaccccaatttcggattttttccaaaatctcccattttggaatttgcctgctgggattccttagactcttttccttcccttttctgttctagttttttacttttgtttatttttatggtaactgctttctgttccaaattcagtttcataaaaggagaaccagcacagcttagatttcatagttcagaatttagtgtatccataatgcattcttctctgttgtcgtaaagatttgggtgaacaaacaatgaaaactctttgctgctgcccatgtttcaaatacttagagcagtgaagactagaaaattagactgtgattcagaaaatgttctgtttgctgtggaactacattactgtacagggttatctgcaagtgaggtgtgtcacaatgagattgaatttcactgtctttaattctgtatctgtagacggctcagtatagataccctacgctgtccagaaaggtttggggcagaaaggactcctcctttttccatgccctaaacagacctgacaggtgaggtctgttccttttatataagtggacaaattttgagttgccacaggaggggaagtagggaggggggaaatacagttctgctctggttgtttctgttccaaatgattccatccacctttcccaatcggccttacttctcactaatttgtaggaaaaagcaagttcgtctgttgtgcgaatgactgaatgggacagagttgatttttttttttttttcctttgtgcttagttaggaaggcagtaggatgtggcctgcatgtactgtatattacagatatttgtcatgctgggatttccaactcgaatctgtgtgaaactttcattccttcagatttggcttgacaaaggcaggaggtacaaaagaagggctggtattgttctcacactggtctgctgtcgctctcagttctcgataggtcagagcagaggtggaaaaacagcatgtacggattttcagttacttaatcaaaactcaaatgtgagtgtttttatctttttacctttcatacactagccttggcctctttcctcagccttaagaaccatctgccaaaaattactgatcctcgcatgatggcagccatagtgcatagctactaaaatcagtgaccttgaacatatcttagatggggagcctcgggaaaaggtagaggagtcacgttaccatttacatgttttaaagaaagaagtgtggggattttcactgaaacgtctaggaaatctagaagtagtcctgaaggacagaaactaaactcttaccatatgtttggtaagactccagactccagctaacagtccctatggaaagatggcatcaaaaaagatagatctatatatatatataaatatatattctattacattttcagtgagtaattttggattttgcaaggtgcatttttactattgttacattatgtggaaaacttatgctgatttatttaagggggaaaaagtgtcaactctttgttatttgaaaacatgtttatttttcttgtctttattttaacctttgatagaaccattgcaatatgggggccttttgggaacggactggtatgtaaaagaaaatccattatcgagcagcattttatttacccctcccctatccctaggcacttaaccaagacaaaaagccacaatgaacatccctttttcaatgaattttataatctgcagctctattccgagcccttagcacccattccgaccatagtataatcatatcaaagggtgagaatcatttagcatgttgttgaaaggttttttttcagttgttctttttagaaaaaaagaaaaacaaaaacaaaaacaaaaaaaaaaaatcacaccattgctcacagaattggcatctcatttttgggacctcccatctttctgttttgaaaagtgtacagtagtgcagtgttcctgatgtaactttatggcttacaatgttgacatgtctcaggttcatgtgttgcgattggtgttttccgtctcaggtagattgcaaagtgtaggccccacacattggaaaaaataataataaaacaaagcaaaaacaggaaattatggattttagttgtatattggtttatgtattttttcttaagtatacagtgcactgtttgaaatgtattgttgagtattactttgtacaggttgatcactttttttagagtgaagaaagaacaaacttgttttttgtgttttttaaaggaatataaaataatgaaggatgtataattgatgccaaataagcttgttctttagtcacaccgacgtcttatttttccctttaggccagttctgtttttaaggtgtacatggacaatgttacagtgtaagaaactccatatccatatgttcccattcgcattttgtattggttcatgtataccatttttacaaaaaaaaaaagaaaaaaaagaagtactataaaatatctgtcttcttaataaaaaaaaattaatgttacaaagtga

SEQ ID NO: 40 - DNA, 1123 bp, Homo sapiens
ggttgggctcgcggcgctgtgattggtctgcccggactccgcctccagcgcatgtcattagcatctcattagctgtccgctcgggctccggaggcagccaacgccgccagtctgaggcaggtgcccgacatggcgagtgtagtgctgccgagcggatcccagtgtgcggcggcagcggcggcggcggcgcctcccgggctccggctccggcttctgctgttgctcttctccgccgcggcactgatccccacaggtgatgggcagaatctgtttacgaaagacgtgacagtgatcgagggagaggttgcgaccatcagttgccaagtcaataagagtgacgactctgtgattcagctactgaatcccaacaggcagaccatttatttcagggacttcaggcctttgaaggacagcaggtttcagttgctgaatttttctagcagtgaactcaaagtatcattgacaaacgtctcaatttctgatgaaggaagatacttttgccagctctataccgatcccccacaggaaagttacaccaccatcacagtcctggtcccaccacgtaatctgatgatcgatatccagaaagacactgcggtggaaggtgaggagattgaagtcaactgcactgctatggccagcaagccagccacgactatcaggtggttcaaagggaacacagagctaaaaggcaaatcggaggtggaagagtggtcagacatgtacactgtgaccagtcagctgatgctgaaggtgcacaaggaggacgatggggtcccagtgatctgccaggtggagcaccctgcggtcactggaaacctgcagacccagcggtatctagaagtacagtataagcctcaagtgcacattcagatgacttatcctctacaaggcttaacccgggaaggggacgcgcttgagttaacatgtgaagccatcgggaagccccagcctgtgatggtaacttgggtgagagtcgatgatgaaatgcctcaacacgccgtactgtctgggcccaacctgttcatcaataacctaaacaaaacagataatggtacataccgctgtgaagcttcaaacatagtggggaaagctcactcggattatatgctgtatgtatacg

SEQ ID NO: 41 - DNA, 110 bp, Homo sapiens
gacgaagacagtccctggatcaccgacagcacagacagaatccctgctaccaccttgcctccccgattgaaagagatgaaaagccaggaatcggctgcaggttccaaact

SEQ ID NO: 42 - DNA, 200 bp, Homo sapiens
ctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcg

SEQ ID NO: 43 - DNA, 166 bp, Homo sapiens
atttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcag

SEQ ID NO: 44 - DNA, 134 bp, Homo sapiens
gtatgggttcatagaagggcacgtggtgattccccggatccaccccaactccatctgtgcagcaaacaacacaggggtgtacatcctcacatccaacacctcccagtatgacacatattgcttcaatgcttcag

SEQ ID NO: 45 - DNA, 69 bp, Homo sapiens
ctccacctgaagaagattgtacatcagtcacagacctgcccaatgcctttgatggaccaattaccataa

SEQ ID NO: 46 - DNA, 231 bp, Homo sapiens
ctattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctacca

SEQ ID NO: 47 - DNA, 129 bp, Homo sapiens
ctttgatgagcactagtgctacagcaactgagacagcaaccaagaggcaagaaacctgggattggttttcatggttgtttctaccatcagagtcaaagaatcatcttcacacaacaacacaaatggctg

SEQ ID NO: 48 - DNA, 126 bp, Homo sapiens
gtacgtcttcaaataccatctcagcaggctgggagccaaatgaagaaaatgaagatgaaagagacagacacctcagtttttctggatcaggcattgatgatgatgaagattttatctccagcacca

SEQ ID NO: 49 - DNA, 114 bp, Homo sapiens
tttcaaccacaccacgggcttttgaccacacaaaacagaaccaggactggacccagtggaacccaagccattcaaatccggaagtgctacttcagacaaccacaaggatgactg

SEQ ID NO: 50 - DNA, 117 bp, Homo sapiens
atgtagacagaaatggcaccactgcttatgaaggaaactggaacccagaagcacaccctcccctcattcaccatgagcatcatgaggaagaagagaccccacattctacaagcacaa

SEQ ID NO: 51 - DNA, 129 bp, Homo sapiens
tccaggcaactcctagtagtacaacggaagaaacagctacccagaaggaacagtggtttggcaacagatggcatgagggatatcgccaaacacccaaagaagactcccattcgacaacagggacagctg

SEQ ID NO: 52 - DNA, 132 bp, Homo sapiens
cagcctcagctcataccagccatccaatgcaaggaaggacaacaccaagcccagaggacagttcctggactgatttcttcaacccaatctcacaccccatgggacgaggtcatcaagcaggaagaaggatgg

SEQ ID NO: 53 - DNA, 102 bp, Homo sapiens
atatggactccagtcatagtataacgcttcagcctactgcaaatccaaacacaggtttggtggaagatttggacaggacaggacctctttcaatgacaacgc

SEQ ID NO: 54 - DNA, 90 bp, Homo sapiens
agcagagtaattctcagagcttctctacatcacatgaaggcttggaagaagataaagaccatccaacaacttctactctgacatcaagca

SEQ ID NO: 55 - DNA, 204 bp, Homo sapiens
ataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcag

SEQ ID NO: 56 - DNA, 63 bp, Homo sapiens
gagaccaagacacattccaccccagtggggggtcccataccactcatggatctgaatcagatg

SEQ ID NO: 57 - DNA, 72 bp, Homo sapiens
gacactcacatgggagtcaagaaggtggagcaaacacaacctctggtcctataaggacaccccaaattccag

SEQ ID NO: 58 - DNA, 79 bp, Homo sapiens
aatggctgatcatcttggcatccctcttggccttggctttgattcttgcagtttgcattgcagtcaacagtcgaagaag

SEQ ID NO: 59 - DNA, 3274 bp, Homo sapiens
gtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacaga

SEQ ID NO: 60 - DNA, 5431 bp, Homo sapiens
ctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacaga

SEQ ID NO: 61 - DNA, 800 bp, Homo sapiens
ctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctacca

SEQ ID NO: 62 - DNA, 88 bp, Homo sapiens
ccgcatcggcgaccttcaggccttccagggccacggcgcgggcaacctggcggcatctacatctacatccaccactgggacaagccat

SEQ ID NO: 63 - DNA, 552 bp, Homo sapiens
agatgcagtagccgaaactgcgcggaggcacagaggccggggagagcgttctgggtccgagggtccaggtaggggttgagccaccatctgaccgcaagctgcgtcgtgtcgccggttctgcaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgg

SEQ ID NO: 64 - DNA, 174 bp, Homo sapiens
gtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaaga

SEQ ID NO: 65 - DNA, 92 bp, Homo sapiens
gcatccgtgtcattctggaccttactcccaactaccggggtgagaactcgtggttctccactcaggttgacactgtggccaccaaggtgaag

SEQ ID NO: 66 - DNA, 69 bp, Homo sapiens
gatgctctggagttttggctgcaagctggcgtggatgggttccaggttcgggacatagagaatctgaag

SEQ ID NO: 67 - DNA, 59 bp, Homo sapiens
gatgcatcctcattcttggctgagtggcaaaatatcaccaagggcttcagtgaagacag

SEQ ID NO: 68 - DNA, 181 bp, Homo sapiens
gctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagt

SEQ ID NO: 69 - DNA, 144 bp, Homo sapiens
ttgtctcaggcaaggctcctgacttccttcttgccggctcaacttctccgactctaccagctgatgctcttcaccctgccagggacccctgttttcagctacggggatgagattggcctggatgcagctgcccttcctggacag

SEQ ID NO: 70 - DNA, 84 bp, Homo sapiens
cctatggaggctccagtcatgctgtgggatgagtccagcttccctgacatcccaggggctgtaagtgccaacatgactgtgaag

SEQ ID NO: 71 - DNA, 530 bp, Homo sapiens
ggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctgacttcagcctgacatggacccactacccttctcctttccttcccaggccctttggcttctgatttttctcttttttaaaaacaaacaaacaaactgttgcagattatgagtgaacccccaaatagggtgttttctgccttcaaataaaagtcacccctgcatggtgaa

SEQ ID NO: 72 - DNA, 1885 bp, Homo sapiens
agatgcagtagccgaaactgcgcggaggcacagaggccggggagagcgttctgggtccgagggtccaggtaggggttgagccaccatctgaccgcaagctgcgtcgtgtcgccggttctgcaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctgacttcagcctgacatggacccactacccttctcctttccttcccaggccctttggcttctgatttttctcttttttaaaaacaaacaaacaaactgttgcagattatgagtgaacccccaaatagggtgttttctgccttcaaataaaagtcacccctgcatggtgaa

SEQ ID NO: 73 - DNA, 1333 bp, Homo sapiens
gtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctgacttcagcctgacatggacccactacccttctcctttccttcccaggccctttggcttctgatttttctcttttttaaaaacaaacaaacaaactgttgcagattatgagtgaacccccaaatagggtgttttctgccttcaaataaaagtcacccctgcatggtgaa

SEQ ID NO: 74 - DNA, 93 bp, Homo sapiens
cataattagcatcatcattattctggctggagcaattgcactcatcattggctttggtatttcagccttgcctccccgattgaaagagatgaa

SEQ ID NO: 75 - DNA, 104 bp, Homo sapiens
gtgagtcaccaaggaaggcagcggcagctccactcagccagtacccagatacgctgggaaccttccccagccatggcttccctggggcagatcctcttctggag

SEQ ID NO: 76 - DNA, 65 bp, Homo sapiens
cataattagcatcatcattattctggctggagcaattgcactcatcattggctttggtatttcag

SEQ ID NO: 77 - DNA, 348 bp, Homo sapiens
ggagacactccatcacagtcactactgtcgcctcagctgggaacattggggaggatggaatcctgagctgcacttttgaacctgacatcaaactttctgatatcgtgatacaatggctgaaggaaggtgttttaggcttggtccatgagttcaaagaaggcaaagatgagctgtcggagcaggatgaaatgttcagaggccggacagcagtgtttgctgatcaagtgatagttggcaatgcctctttgcggctgaaaaacgtgcaactcacagatgctggcacctacaaatgttatatcatcacttctaaaggcaaggggaatgctaaccttgagtataaaactggag

SEQ ID NO: 78 - DNA, 279 bp, Homo sapiens
ccttcagcatgccggaagtgaatgtggactataatgccagctcagagaccttgcggtgtgaggctccccgatggttcccccagcccacagtggtctgggcatcccaagttgaccagggagccaacttctcggaagtctccaataccagctttgagctgaactctgagaatgtgaccatgaaggttgtgtctgtgctctacaatgttacgatcaacaacacatactcctgtatgattgaaaatgacattgccaaagcaacaggggatatcaaagtgacag

SEQ ID NO: 79 - DNA, 170 bp, Homo sapiens
aatcggagatcaaaaggcggagtcacctacagctgctaaactcaaaggcttctctgtgtgtctcttctttctttgccatcagctgggcacttctgcctctcagcccttacctgatgctaaaataatgtgcctcggccacaaaaaagcatgcaaagtcattgttacaacag

SEQ ID NO: 80 - DNA, 1639 bp, Homo sapiens
ggatctacagaactatttcaccaccagatatgacctagttttatatttctgggaggaaatgaattcatatctagaagtctggagtgagcaaacaagagcaagaaacaaaaagaagccaaaagcagaaggctccaatatgaacaagataaatctatcttcaaagacatattagaagttgggaaaataattcatgtgaactagacaagtgtgttaagagtgataagtaaaatgcacgtggagacaagtgcatccccagatctcagggacctccccctgcctgtcacctggggagtgagaggacaggatagtgcatgttctttgtctctgaatttttagttatatgtgctgtaatgttgctctgaggaagcccctggaaagtctatcccaacatatccacatcttatattccacaaattaagctgtagtatgtaccctaagacgctgctaattgactgccacttcgcaactcaggggcggctgcattttagtaatgggtcaaatgattcactttttatgatgcttccaaaggtgccttggcttctcttcccaactgacaaatgccaaagttgagaaaaatgatcataattttagcataaacagagcagtcggcgacaccgattttataaataaactgagcaccttctttttaaacaaacaaatgcgggtttatttctcagatgatgttcatccgtgaatggtccagggaaggacctttcaccttgtctatatggcattatgtcatcacaagctctgaggcttctcctttccatcctgcgtggacagctaagacctcagttttcaatagcatctagagcagtgggactcagctggggtgatttcgccccccatctccgggggaatgtctgaagacaattttggttacctcaatgagggagtggaggaggatacagtgctactaccaactagtggatagaggccagggatgctgctcaacctcctaccatgtacaggacgtctccccattacaactacccaatccgaagtgtcaactgtgtcagggctaagaaaccctggttttgagtagaaaagggcctggaaagaggggagccaacaaatctgtctgcttcctcacattagtcattggcaaataagcattctgtctctttggctgctgcctcagcacagagagccagaactctatcgggcaccaggataacatctctcagtgaacagagttgacaaggcctatgggaaatgcctgatgggattatcttcagcttgttgagcttctaagtttctttcccttcattctaccctgcaagccaagttctgtaagagaaatgcctgagttctagctcaggttttcttactctgaatttagatctccagaccctgcctggccacaattcaaattaaggcaacaaacatataccttccatgaagcacacacagacttttgaaagcaaggacaatgactgcttgaattgaggccttgaggaatgaagctttgaaggaaaagaatactttgtttccagcccccttcccacactcttcatgtgttaaccactgccttcctggaccttggagccacggtgactgtattacatgttgttatagaaaactgattttagagttctgatcgttcaagagaatgattaaatatacatttcctacacca

SEQ ID NO: 81 - DNA, 2605 bp, Homo sapiens
gtgagtcaccaaggaaggcagcggcagctccactcagccagtacccagatacgctgggaaccttccccagccatggcttccctggggcagatcctcttctggagcataattagcatcatcattattctggctggagcaattgcactcatcattggctttggtatttcagggagacactccatcacagtcactactgtcgcctcagctgggaacattggggaggatggaatcctgagctgcacttttgaacctgacatcaaactttctgatatcgtgatacaatggctgaaggaaggtgttttaggcttggtccatgagttcaaagaaggcaaagatgagctgtcggagcaggatgaaatgttcagaggccggacagcagtgtttgctgatcaagtgatagttggcaatgcctctttgcggctgaaaaacgtgcaactcacagatgctggcacctacaaatgttatatcatcacttctaaaggcaaggggaatgctaaccttgagtataaaactggagccttcagcatgccggaagtgaatgtggactataatgccagctcagagaccttgcggtgtgaggctccccgatggttcccccagcccacagtggtctgggcatcccaagttgaccagggagccaacttctcggaagtctccaataccagctttgagctgaactctgagaatgtgaccatgaaggttgtgtctgtgctctacaatgttacgatcaacaacacatactcctgtatgattgaaaatgacattgccaaagcaacaggggatatcaaagtgacagaatcggagatcaaaaggcggagtcacctacagctgctaaactcaaaggcttctctgtgtgtctcttctttctttgccatcagctgggcacttctgcctctcagcccttacctgatgctaaaataatgtgcctcggccacaaaaaagcatgcaaagtcattgttacaacagggatctacagaactatttcaccaccagatatgacctagttttatatttctgggaggaaatgaattcatatctagaagtctggagtgagcaaacaagagcaagaaacaaaaagaagccaaaagcagaaggctccaatatgaacaagataaatctatcttcaaagacatattagaagttgggaaaataattcatgtgaactagacaagtgtgttaagagtgataagtaaaatgcacgtggagacaagtgcatccccagatctcagggacctccccctgcctgtcacctggggagtgagaggacaggatagtgcatgttctttgtctctgaatttttagttatatgtgctgtaatgttgctctgaggaagcccctggaaagtctatcccaacatatccacatcttatattccacaaattaagctgtagtatgtaccctaagacgctgctaattgactgccacttcgcaactcaggggcggctgcattttagtaatgggtcaaatgattcactttttatgatgcttccaaaggtgccttggcttctcttcccaactgacaaatgccaaagttgagaaaaatgatcataattttagcataaacagagcagtcggcgacaccgattttataaataaactgagcaccttctttttaaacaaacaaatgcgggtttatttctcagatgatgttcatccgtgaatggtccagggaaggacctttcaccttgtctatatggcattatgtcatcacaagctctgaggcttctcctttccatcctgcgtggacagctaagacctcagttttcaatagcatctagagcagtgggactcagctggggtgatttcgccccccatctccgggggaatgtctgaagacaattttggttacctcaatgagggagtggaggaggatacagtgctactaccaactagtggatagaggccagggatgctgctcaacctcctaccatgtacaggacgtctccccattacaactacccaatccgaagtgtcaactgtgtcagggctaagaaaccctggttttgagtagaaaagggcctggaaagaggggagccaacaaatctgtctgcttcctcacattagtcattggcaaataagcattctgtctctttggctgctgcctcagcacagagagccagaactctatcgggcaccaggataacatctctcagtgaacagagttgacaaggcctatgggaaatgcctgatgggattatcttcagcttgttgagcttctaagtttctttcccttcattctaccctgcaagccaagttctgtaagagaaatgcctgagttctagctcaggttttcttactctgaatttagatctccagaccctgcctggccacaattcaaattaaggcaacaaacatataccttccatgaagcacacacagacttttgaaagcaaggacaatgactgcttgaattgaggccttgaggaatgaagctttgaaggaaaagaatactttgtttccagcccccttcccacactcttcatgtgttaaccactgccttcctggaccttggagccacggtgactgtattacatgttgttatagaaaactgattttagagttctgatcgttcaagagaatgattaaatatacatttcctacacca

SEQ ID NO: 82 - DNA, 169 bp, Homo sapiens
gtgagtcaccaaggaaggcagcggcagctccactcagccagtacccagatacgctgggaaccttccccagccatggcttccctggggcagatcctcttctggagcataattagcatcatcattattctggctggagcaattgcactcatcattggctttggtatttcag

SEQ ID NO: 83 - DNA, 148 bp, Homo sapiens
ctggctggagattaatccggacactggtgccatttccactcgggctgagctggacagggaggattttgagcacgtgaagaacagcacgtacacagccctaatcatagctacagacaatgccttgcctccccgattgaaagagatgaaa

SEQ ID NO: 84 - DNA, 172 bp, Homo sapiens
agtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcag

SEQ ID NO: 85 - DNA, 115 bp, Homo sapiens
gtctcctcttggctctgccaggagccggagccctgccaccctggctttgacgccgagagctacacgttcacggtgccccggcgccacctggagagaggccgcgtcctgggcagag

SEQ ID NO: 86 - DNA, 224 bp, Homo sapiens
tgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcag

SEQ ID NO: 87 - DNA, 144 bp, Homo sapiens
gcctccgtttctggaatccaagcagaattgctcacatttcccaactcctctcctggcctcagaagacagaagagagactgggttattcctcccatcagctgcccagaaaatgaaaaaggcccatttcctaaaaacctggttcag

SEQ ID NO: 88 - DNA, 156 bp, Homo sapiens
atcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacact

SEQ ID NO: 89 - DNA, 145 bp, Homo sapiens
ctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccag

SEQ ID NO: 90 - DNA, 176 bp, Homo sapiens
gaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagag

SEQ ID NO: 91 - DNA, 129 bp, Homo sapiens
agtttccctacgtataccctggtggttcaagctgctgaccttcaaggtgaggggttaagcacaacagcaacagctgtgatcacagtcactgacaccaacgataatcctccgatcttcaatcccaccacg

SEQ ID NO: 92 - DNA, 250 bp, Homo sapiens
tacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaaggtttgtatggtacctggcaagatgcagaaactggcatcctcacagctgttcatacccttgtcccctg

SEQ ID NO: 93 - DNA, 245 bp, Homo sapiens
ggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataac

SEQ ID NO: 94 - DNA, 146 bp, Homo sapiens
atatcggatttggagagacactgccaactggctggagattaatccggacactggtgccatttccactcgggctgagctggacagggaggattttgagcacgtgaagaacagcacgtacacagccctaatcatagctacagacaatg

SEQ ID NO: 95 - DNA, 225 bp, Homo sapiens
gttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaa

SEQ ID NO: 96 - DNA, 228 bp, Homo sapiens
cccaagaatctatcattttgaagccaaagatggccttagaggtgggtgactacaaaatcaatctcaagctcatggataaccagaataaagaccaagtgaccaccttagaggtcagcgtgtgtgactgtgaaggggccgctggcgtctgtaggaaggcacagcctgtcgaagcaggattgcaaattcctgccattctggggattcttggaggaattcttgctttgctaa

SEQ ID NO: 97 - DNA, 131 bp, Homo sapiens
ttctgattctgctgctcttgctgtttcttcggaggagagcggtggtcaaagagcccttactgcccccagaggatgacacccgggacaacgtttattactatgatgaagaaggaggcggagaagaggaccag

SEQ ID NO: 98 - DNA, 144 bp, Homo sapiens
gactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaa

SEQ ID NO: 99 - DNA, 2248 bp, Homo sapiens
aatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactaggggactcgagagaggcgggccccagacccatgtgctgggaaatgcagaaatcacgttgctggtggtttttcagctcccttcccttgagatgagtttctggggaaaaaaaagagactggttagtgatgcagttagtatagctttatactctctccactttatagctctaataagtttgtgttagaaaagtttcgacttatttcttaaagcttttttttttttcccatcactctttacatggtggtgatgtccaaaagatacccaaattttaatattccagaagaacaactttagcatcagaaggttcacccagcaccttgcagattttcttaaggaattttgtctcacttttaaaaagaaggggagaagtcagctactctagttctgttgttttgtgtatataattttttaaaaaaaatttgtgtgcttctgctcattactacactggtgtgtccctctgccttttttttttttttaagacagggtctcattctatcggccaggctggagtgcagtggtgcaatcacagctcactgcagccttgtcctcccaggctcaagctatccttgcacctcagcctcccaagtagctgggaccacaggcatgcaccactacgcatgactaattttttaaatatttgagacggggtctccctgtgttacccaggctggtctcaaactcctgggctcaagtgatcctcccatcttggcctcccagagtattgggattacagacatgagccactgcacctgcccagctccccaactccctgccattttttaagagacagtttcgctccatcgcccaggcctgggatgcagtgatgtgatcatagctcactgtaacctcaaactctggggctcaagcagttctcccaccagcctcctttttatttttttgtacagatggggtcttgctatgttgcccaagctggtcttaaactcctggcctcaagcaatccttctgccttggccccccaaagtgctgggattgtgggcatgagctgctgtgcccagcctccatgttttaatatcaactctcactcctgaattcagttgctttgcccaagataggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccaggacttagaatagtgcctaaagtgctgcagccaaagacagagcggaactatgaaaagtgggcttggagatggcaggagagcttgtcattgagcctggcaatttagcaaactgatgctgaggatgattgaggtgggtctacctcatctctgaaaattctggaaggaatggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaatttttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtattttctttgggggtggaaaaggaaaacaattcaagctgagaaaagtattctcaaagatgcatttttataaattttattaaacaattttgttaaa

SEQ ID NO: 100 - DNA, 4878 bp, Homo sapiens
agtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaaggtttgtatggtacctggcaagatgcagaaactggcatcctcacagctgttcatacccttgtcccctgggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgctggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactaggggactcgagagaggcgggccccagacccatgtgctgggaaatgcagaaatcacgttgctggtggtttttcagctcccttcccttgagatgagtttctggggaaaaaaaagagactggttagtgatgcagttagtatagctttatactctctccactttatagctctaataagtttgtgttagaaaagtttcgacttatttcttaaagcttttttttttttcccatcactctttacatggtggtgatgtccaaaagatacccaaattttaatattccagaagaacaactttagcatcagaaggttcacccagcaccttgcagattttcttaaggaattttgtctcacttttaaaaagaaggggagaagtcagctactctagttctgttgttttgtgtatataattttttaaaaaaaatttgtgtgcttctgctcattactacactggtgtgtccctctgccttttttttttttttaagacagggtctcattctatcggccaggctggagtgcagtggtgcaatcacagctcactgcagccttgtcctcccaggctcaagctatccttgcacctcagcctcccaagtagctgggaccacaggcatgcaccactacgcatgactaattttttaaatatttgagacggggtctccctgtgttacccaggctggtctcaaactcctgggctcaagtgatcctcccatcttggcctcccagagtattgggattacagacatgagccactgcacctgcccagctccccaactccctgccattttttaagagacagtttcgctccatcgcccaggcctgggatgcagtgatgtgatcatagctcactgtaacctcaaactctggggctcaagcagttctcccaccagcctcctttttatttttttgtacagatggggtcttgctatgttgcccaagctggtcttaaactcctggcctcaagcaatccttctgccttggccccccaaagtgctgggattgtgggcatgagctgctgtgcccagcctccatgttttaatatcaactctcactcctgaattcagttgctttgcccaagataggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccaggacttagaatagtgcctaaagtgctgcagccaaagacagagcggaactatgaaaagtgggcttggagatggcaggagagcttgtcattgagcctggcaatttagcaaactgatgctgaggatgattgaggtgggtctacctcatctctgaaaattctggaaggaatggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaatttttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtattttctttgggggtggaaaaggaaaacaattcaagctgagaaaagtattctcaaagatgcatttttataaattttattaaacaattttgttaaa

SEQ ID NO: 101 - DNA, 1902 bp, Homo sapiens
agtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaaggtttgtatggtacctggcaagatgcagaaactggcatcctcacagctgttcatacccttgtcccctgggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatg

SEQ ID NO: 102 - DNA, 101 bp, Homo sapiens
atggcgctcctgctgtgcttcgtgctcctgtgcggagtagtggccttgcctccccgattgaaagagatgaaaagccaggaatcggctgcaggttccaaact

SEQ ID NO: 103 - DNA, 130 bp, Homo sapiens
agtcgggagcgcgcgaggcgcggggagcctgggaccaggagcgagagccgcctacctgcagccgccgcccacggcacggcagccaccatggcgctcctgctgtgcttcgtgctcctgtgcggagtagtgg

SEQ ID NO: 104 - DNA, 167 bp, Homo sapiens
atttcgccagaagtttgagtatcactactcctgaagagatgattgaaaaagccaaaggggaaactgcctatctgccatgcaaatttacgcttagtcccgaagaccagggaccgctggacatcgagtggctgatatcaccagctgataatcagaaggtggatcaagtg

SEQ ID NO: 105 - DNA, 205 bp, Homo sapiens
attattttatattctggagacaaaatttatgatgactactatccagatctgaaaggccgagtacattttacgagtaatgatctcaaatctggtgatgcatcaataaatgtaacgaatttacaactgtcagatattggcacatatcagtgcaaagtgaaaaaagctcctggtgttgcaaataagaagattcatctggtagttcttg

SEQ ID NO: 106 - DNA, 156 bp, Homo sapiens
ttaagccttcaggtgcgagatgttacgttgatggatctgaagaaattggaagtgactttaagataaaatgtgaaccaaaagaaggttcacttccattacagtatgagtggcaaaaattgtctgactcacagaaaatgcccacttcatggttagcag

SEQ ID NO: 107 - DNA, 4673 bp, Homo sapiens
ggaagatgtgccacctccaaagagccgtacgtccactgccagaagctacatcggcagtaatcattcatccctggggtccatgtctccttccaacatggaaggatattccaagactcagtataaccaagtaccaagtgaagactttgaacgcactcctcagagtccgactctcccacctgctaaggtagctgcccctaatctaagtcgaatgggtgcgattcctgtgatgattccagcacagagcaaggatgggtctatagtatagagcctccatatgtctcatctgtgctctccgtgttcctttcctttttttgatatatgaaaacctattctggtctaaattgtgttactagcctcaaaatacatcaaaaaataagttaatcaggaactgtacggaatatatttttaaaaatttttgtttggttatatcgaaatagttacaggcactaaagttagtaaagaaaagtttaccatctgaaaaagctggattttctttaagaggttgattataaagttttctaaatttatcagtacctaagtaagatgtagcgctttgaatatgaaatcataggtgaagacatgggtgaacttacttgcataccaagttgatacttgaataaccatctgaaagtggtacttgatcatttttaccattatttttaggatgtgtatttcatttatttatggcccaccagtctcccccaaattagtacagaaatatccatgacaaaattacttacgtatgtttgtacttggttttacagctcctttgaaaactctgtgtttggaatatctctaaaaacatagaaaacactacagtggtttagaaattactaattttacttctaagtcattcataaaccttgtctatgaaatgacttcttaaatatttagttgatagactgctacaggtaatagggacttagcaagctcttttatatgctaaaggagcatctatcagattaagttagaacatttgctgtcagccacatattgagatgacactaggtgcaatagcagggatagattttgttggtgagtagtctcatgccttgagatctgtggtggtcttcaaaatggtggccagccagatcaaggatgtagtatctcatagttcccaggtgatatttttcttattagaaaaatattataactcatttgttgtttgacacttatagattgaaatttcctaatttattctaaattttaagtggttctttggttccagtgctttatgttgttgttgtttttggatggtgttacatattatatgttctagaaacatgtaatcctaaatttaccctcttgaatataatccctggatgatattttttatcataaatgcagaataatcaaatacattttaagcaagttaagtgtcctccatcaattctgtattccagacttgggaggatgtacagttgctgttgtgtgatcaaacatgtctctgtgtagttccagcaaatcaagctgagctttgaaaaagtttgtcttagttttgtgaaggtgatttattcttaaaaaaaaaaaagaaagaaaaagaaaaaaagataagaaggaggagtaaagggactactcctccttgccaaatgtgctaaatatcattttaggagaagaaagtgggtttattgtatttcccttaagattgtgagggagtgtggatacagtagaatgagccaacagtttctttataataaatacggtctgcaataaattatttcactagctctaaaacctttccctagattttagtagggagttggtttctgttaatatctttgggtgctgtggtggtaaatgctatattatgaacggtggcatgtatttacagttagagtattgtgtgtacactttttaatggtaaacttaagctgaatgtgtaatggatttgtgtatagttttacatatttggaagcattttaaaaacaggttttaaccttatgtaaaattacttttatactcgtgttaacattttcatctgtgccttttggtaatttaatttctattatgaatttctggtgcctatgagctagctatcacctacctgaaaggtgcttagaggtgaaggtactgtttctaaaaacacatcactgtgatacctttctatcctcacattttcaagcttgcctcttttctgttctttgtggatataacttaagcaattgtgttattcataaaggtttagaaatttcaatattcccaacactctatgtttctgattttataacagtagccatttttgaaagtcagatgtttggcctgttttatatgaataaagtttatttataaaatattataaaaaataagtaaataaacagaacattaataataaagttttggttcttcctattcctgactttcatataatgaaaattatcctattgatctaagtagaagttatcatagaaagtggacacgtataagactttccttcctttttttttttaataacatatgaggaacaagacttctcttcccatatacttcatattttagaggacattgttttaaaggcttatgtctcactgtaaaattctgtcagccaaatagtaccaatacgttttcaagtagttctcactgataatttagttgaaccagagatcaaatatttgctcccgaattactactggtaatcaagtagttgaacaaaaaattactaaagcatttccgttagatcagtcaaggacagtactgcatctttttttttttttttttttttaagacggagtctcggtctgtcacccaggctggagtgcagtggcgggatctcggctcactgcaagctccgcctcccaggttcacgccattctcctgcctcagcctcccgagcagctgggactacaggctcccatcaccacgctcggctaagtttttgtaattttagtagagacagggtttcaccgtgttagccaggatggtctcgatctcctgacctcgtgatctgcctgcctcggcctcccaaagtgctgggattacaggcgtgaaccactgcacccggcccagtactgcatcttaacagcaaagccattttattctactttataactgagagacttgataccatccatctctttaggttacagaggataatttgaagagaaatgttactgtagaatatatagttctgtactttttttttttttttttaagagatagggtttcactattgctcaggctggtctcaaactgctgggctcaggagatcctcctgccttggcctcccaaattgctgggattacaggtgtgagccgcagcatccagccagttctgtactttgaatatggagtagtttacagctatttttttttcttactggtaatcttaactaatatgattcccttgttagagagcctctcactcccccacccccaaaaatgtctactattcatgacagtaaccaattattctggacaaattgcttctttttaatttgagctatctgccatggactttctaaaatggaaacacagcctgagtgtatcttagggagagtttgattgaaaaaatccaaatcactatccatatagatcatggatataaagagatacctgatttttattaaaaagatactttttcaaatttaagagttaatcttggaaatttggaacaagtaaaggggcaagtaaaccttttgatgaaatataaaaggaactcattgcatgaagttgactatcaaattctgtgatgtgtggcttcttaaaaatattctcagtgtcttttgtgtgcgtgcagcatgtacatttgatgttatgtgaatgttgagttttttcttctaattttcacttcagcagtgtttagggctttcagatgccttattccagtgtgaacagaaaaagttcatattttatgtggttaatgctttgatgtgtcacataaagagtagtttgtagaaaatgttggcacaattttaacttcttagtggcttgtgacattatatattatatatatatatgtacatatatctttataacattcctgtgtttagtagtgtaaatgttctgggcaagttttaatattttgaatgcctttggatattccagcaataaaggcatcatgttctgcaataggatttcttactcatttacctattttaacactaaaatagaccacaactgagcacaaattccttttataaatgttatagaagcagggaagaataataaacacatttgtgaattgtggttcagtttatttatctttagggaaggctgatcatttatcttatagcagataaccccagcctcttattcattatggttaacttttataatttatcttattttataatttaagaatatagtacatatcagttgggtttggttttggtcatcgagactaaaagctccatcaaaacagaactttgtgttttctgctaacttatttaatgacacaagttttaagagaaccacaattcattgattcacttattcttttccctaattgtgaattttagtgataaatacacctgtactactgaggaaaatattctgacacttcacgtgtgcaaagtatagaactgacagtgtcagtttcagattttgtatgtacgatttctggcttatatatccaatggtgcagattttgaaatttgtaagaacaaaatttgttaagaaaaacaacttgctctagttttgtgaccttgtgtacttttgaaataaaatcaagaaagcagt

SEQ ID NO: 108 - DNA, 5331 bp, Homo sapiens
agtcgggagcgcgcgaggcgcggggagcctgggaccaggagcgagagccgcctacctgcagccgccgcccacggcacggcagccaccatggcgctcctgctgtgcttcgtgctcctgtgcggagtagtggatttcgccagaagtttgagtatcactactcctgaagagatgattgaaaaagccaaaggggaaactgcctatctgccatgcaaatttacgcttagtcccgaagaccagggaccgctggacatcgagtggctgatatcaccagctgataatcagaaggtggatcaagtgattattttatattctggagacaaaatttatgatgactactatccagatctgaaaggccgagtacattttacgagtaatgatctcaaatctggtgatgcatcaataaatgtaacgaatttacaactgtcagatattggcacatatcagtgcaaagtgaaaaaagctcctggtgttgcaaataagaagattcatctggtagttcttgttaagccttcaggtgcgagatgttacgttgatggatctgaagaaattggaagtgactttaagataaaatgtgaaccaaaagaaggttcacttccattacagtatgagtggcaaaaattgtctgactcacagaaaatgcccacttcatggttagcagggaagatgtgccacctccaaagagccgtacgtccactgccagaagctacatcggcagtaatcattcatccctggggtccatgtctccttccaacatggaaggatattccaagactcagtataaccaagtaccaagtgaagactttgaacgcactcctcagagtccgactctcccacctgctaaggtagctgcccctaatctaagtcgaatgggtgcgattcctgtgatgattccagcacagagcaaggatgggtctatagtatagagcctccatatgtctcatctgtgctctccgtgttcctttcctttttttgatatatgaaaacctattctggtctaaattgtgttactagcctcaaaatacatcaaaaaataagttaatcaggaactgtacggaatatatttttaaaaatttttgtttggttatatcgaaatagttacaggcactaaagttagtaaagaaaagtttaccatctgaaaaagctggattttctttaagaggttgattataaagttttctaaatttatcagtacctaagtaagatgtagcgctttgaatatgaaatcataggtgaagacatgggtgaacttacttgcataccaagttgatacttgaataaccatctgaaagtggtacttgatcatttttaccattatttttaggatgtgtatttcatttatttatggcccaccagtctcccccaaattagtacagaaatatccatgacaaaattacttacgtatgtttgtacttggttttacagctcctttgaaaactctgtgtttggaatatctctaaaaacatagaaaacactacagtggtttagaaattactaattttacttctaagtcattcataaaccttgtctatgaaatgacttcttaaatatttagttgatagactgctacaggtaatagggacttagcaagctcttttatatgctaaaggagcatctatcagattaagttagaacatttgctgtcagccacatattgagatgacactaggtgcaatagcagggatagattttgttggtgagtagtctcatgccttgagatctgtggtggtcttcaaaatggtggccagccagatcaaggatgtagtatctcatagttcccaggtgatatttttcttattagaaaaatattataactcatttgttgtttgacacttatagattgaaatttcctaatttattctaaattttaagtggttctttggttccagtgctttatgttgttgttgtttttggatggtgttacatattatatgttctagaaacatgtaatcctaaatttaccctcttgaatataatccctggatgatattttttatcataaatgcagaataatcaaatacattttaagcaagttaagtgtcctccatcaattctgtattccagacttgggaggatgtacagttgctgttgtgtgatcaaacatgtctctgtgtagttccagcaaatcaagctgagctttgaaaaagtttgtcttagttttgtgaaggtgatttattcttaaaaaaaaaaaagaaagaaaaagaaaaaaagataagaaggaggagtaaagggactactcctccttgccaaatgtgctaaatatcattttaggagaagaaagtgggtttattgtatttcccttaagattgtgagggagtgtggatacagtagaatgagccaacagtttctttataataaatacggtctgcaataaattatttcactagctctaaaacctttccctagattttagtagggagttggtttctgttaatatctttgggtgctgtggtggtaaatgctatattatgaacggtggcatgtatttacagttagagtattgtgtgtacactttttaatggtaaacttaagctgaatgtgtaatggatttgtgtatagttttacatatttggaagcattttaaaaacaggttttaaccttatgtaaaattacttttatactcgtgttaacattttcatctgtgccttttggtaatttaatttctattatgaatttctggtgcctatgagctagctatcacctacctgaaaggtgcttagaggtgaaggtactgtttctaaaaacacatcactgtgatacctttctatcctcacattttcaagcttgcctcttttctgttctttgtggatataacttaagcaattgtgttattcataaaggtttagaaatttcaatattcccaacactctatgtttctgattttataacagtagccatttttgaaagtcagatgtttggcctgttttatatgaataaagtttatttataaaatattataaaaaataagtaaataaacagaacattaataataaagttttggttcttcctattcctgactttcatataatgaaaattatcctattgatctaagtagaagttatcatagaaagtggacacgtataagactttccttcctttttttttttaataacatatgaggaacaagacttctcttcccatatacttcatattttagaggacattgttttaaaggcttatgtctcactgtaaaattctgtcagccaaatagtaccaatacgttttcaagtagttctcactgataatttagttgaaccagagatcaaatatttgctcccgaattactactggtaatcaagtagttgaacaaaaaattactaaagcatttccgttagatcagtcaaggacagtactgcatctttttttttttttttttttttaagacggagtctcggtctgtcacccaggctggagtgcagtggcgggatctcggctcactgcaagctccgcctcccaggttcacgccattctcctgcctcagcctcccgagcagctgggactacaggctcccatcaccacgctcggctaagtttttgtaattttagtagagacagggtttcaccgtgttagccaggatggtctcgatctcctgacctcgtgatctgcctgcctcggcctcccaaagtgctgggattacaggcgtgaaccactgcacccggcccagtactgcatcttaacagcaaagccattttattctactttataactgagagacttgataccatccatctctttaggttacagaggataatttgaagagaaatgttactgtagaatatatagttctgtactttttttttttttttttaagagatagggtttcactattgctcaggctggtctcaaactgctgggctcaggagatcctcctgccttggcctcccaaattgctgggattacaggtgtgagccgcagcatccagccagttctgtactttgaatatggagtagtttacagctatttttttttcttactggtaatcttaactaatatgattcccttgttagagagcctctcactcccccacccccaaaaatgtctactattcatgacagtaaccaattattctggacaaattgcttctttttaatttgagctatctgccatggactttctaaaatggaaacacagcctgagtgtatcttagggagagtttgattgaaaaaatccaaatcactatccatatagatcatggatataaagagatacctgatttttattaaaaagatactttttcaaatttaagagttaatcttggaaatttggaacaagtaaaggggcaagtaaaccttttgatgaaatataaaaggaactcattgcatgaagttgactatcaaattctgtgatgtgtggcttcttaaaaatattctcagtgtcttttgtgtgcgtgcagcatgtacatttgatgttatgtgaatgttgagttttttcttctaattttcacttcagcagtgtttagggctttcagatgccttattccagtgtgaacagaaaaagttcatattttatgtggttaatgctttgatgtgtcacataaagagtagtttgtagaaaatgttggcacaattttaacttcttagtggcttgtgacattatatattatatatatatatgtacatatatctttataacattcctgtgtttagtagtgtaaatgttctgggcaagttttaatattttgaatgcctttggatattccagcaataaaggcatcatgttctgcaataggatttcttactcatttacctattttaacactaaaatagaccacaactgagcacaaattccttttataaatgttatagaagcagggaagaataataaacacatttgtgaattgtggttcagtttatttatctttagggaaggctgatcatttatcttatagcagataaccccagcctcttattcattatggttaacttttataatttatcttattttataatttaagaatatagtacatatcagttgggtttggttttggtcatcgagactaaaagctccatcaaaacagaactttgtgttttctgctaacttatttaatgacacaagttttaagagaaccacaattcattgattcacttattcttttccctaattgtgaattttagtgataaatacacctgtactactgaggaaaatattctgacacttcacgtgtgcaaagtatagaactgacagtgtcagtttcagattttgtatgtacgatttctggcttatatatccaatggtgcagattttgaaatttgtaagaacaaaatttgttaagaaaaacaacttgctctagttttgtgaccttgtgtacttttgaaataaaatcaagaaagcagt

SEQ ID NO: 109 - DNA, 268 bp, Homo sapiens
gggagctgttcaaaaaacgagctctttctactcctgtagtagaaaaacgttcagcatcttctgagtcatcatcatcatcgtcaaagaagaagaaaacaaaggtagaacatggaggatcgtcaggctctaaacaaaattctgccttgcctccccgattgaaagagatgaaaagccaggaatcggctgcaggttccaaactagtccttcggtgtgaaaccagttctgaatactcctctctcagattcaagtggttcaagaatgggaatga

SEQ ID NO: 110 - DNA, 264 bp, Homo sapiens
actgagctcctagcacccgatcgggaagtggcgggcggagtcccgggtccagtcgccgcctcagctaccgccgctgccgccgccgccgccgccaccgccagtggtgagaccccgacctggcgggtcagcgctgggcgtgcgtgcgggcaggcgggggcgctgacgagaagcaggaagagggtgcagtgccggcgtgggcggccggccgaggcggaggcgcaggaagggggcggcgagtcgtgcgaggctgcccttctcactcag

SEQ ID NO: 111 - DNA, 170 bp, Homo sapiens
cattatggatccaagcctgttgagagaaagggagctgttcaaaaaacgagctctttctactcctgtagtagaaaaacgttcagcatcttctgagtcatcatcatcatcgtcaaagaagaagaaaacaaaggtagaacatggaggatcgtcaggctctaaacaaaattctg

SEQ ID NO: 112 - DNA, 92 bp, Homo sapiens
atcatagcaatggatcatttaacttgaaagctttgtcaggaagctctggatataagtttggtgttcttgctaagattgtgaattacatgaag

SEQ ID NO: 113 - DNA, 108 bp, Homo sapiens
acacggcatcagcgaggagatacgcatcctctaaccttagatgaaattttggatgaaacacaacatttagatattggactcaagcagaaacaatggctaatgactgag

SEQ ID NO: 114 - DNA, 183 bp, Homo sapiens
gctttagtcaacaatcccaaaattgaagtaatagatgggaagtatgctttcaagcccaagtacaacgtgagagataagaaggccctacttaggctcttagatcagcatgaccagcgaggattaggaggaattcttttagaagacatagaagaagcactgcccaattcccagaaagctgtcaag

SEQ ID NO: 115 - DNA, 94 bp, Homo sapiens
gctttgggggaccagatactatttgtaaatcgtcccgataagaagaaaatacttttcttcaatgataagagctgtcagttttctgtggatgaag

SEQ ID NO: 116 - DNA, 116 bp, Homo sapiens
aatttcagaaactgtggaggagtgtcactgtagattccatggacgaggagaaaattgaagaatatctgaagcgacagggtatttcttccatgcaggaatctggaccaaagaaagtg

SEQ ID NO: 117 - DNA, 720 bp, Homo sapiens
gcccctattcagagaaggaaaaagcctgcttcacagaaaaagcgacgctttaagactcataacgaacacttggctggagtgctgaaggattactctgacattacttccagcaaatagggaacagttttgccctggaacagagttacagatacacaatcaagagtgttcttgctgatgctcggggtctgaagactgtcttcctatctgcttcttgcggctgaggagaggagcagttcagtttacaaaacaagtgcaaattaccaaactcaaagcttatttgagtagaatgggctcatgggcaatgtgatgttccctgttaaccttctgttactccctgggagaaaggcgctgagcgtggcatgcaggtgtctttgctgtgtttttctccacttctaaatggttcctggttcctttcttcctcgtttgttactttagagcaagtttgcccatagtcttgaatgcaatatttgtttattccaaaagaacatatttataataaaatcactgtagaaggatttttaagatgttagtgaattctgtttcttttcattctcggaaatggcaggaagcagctccagtctctgatttccatgggtcacgtgctggggatgtgatgaagcctgcagtctgcactgtgttgctgagcacatggatttcaccactggaacacaggtgtgctgcttgttagcaagcagagcaataaagatgtgctggatgtca

SEQ ID NO: 118 - DNA, 1747 bp, Homo sapiens
actgagctcctagcacccgatcgggaagtggcgggcggagtcccgggtccagtcgccgcctcagctaccgccgctgccgccgccgccgccgccaccgccagtggtgagaccccgacctggcgggtcagcgctgggcgtgcgtgcgggcaggcgggggcgctgacgagaagcaggaagagggtgcagtgccggcgtgggcggccggccgaggcggaggcgcaggaagggggcggcgagtcgtgcgaggctgcccttctcactcagcattatggatccaagcctgttgagagaaagggagctgttcaaaaaacgagctctttctactcctgtagtagaaaaacgttcagcatcttctgagtcatcatcatcatcgtcaaagaagaagaaaacaaaggtagaacatggaggatcgtcaggctctaaacaaaattctgatcatagcaatggatcatttaacttgaaagctttgtcaggaagctctggatataagtttggtgttcttgctaagattgtgaattacatgaagacacggcatcagcgaggagatacgcatcctctaaccttagatgaaattttggatgaaacacaacatttagatattggactcaagcagaaacaatggctaatgactgaggctttagtcaacaatcccaaaattgaagtaatagatgggaagtatgctttcaagcccaagtacaacgtgagagataagaaggccctacttaggctcttagatcagcatgaccagcgaggattaggaggaattcttttagaagacatagaagaagcactgcccaattcccagaaagctgtcaaggctttgggggaccagatactatttgtaaatcgtcccgataagaagaaaatacttttcttcaatgataagagctgtcagttttctgtggatgaagaatttcagaaactgtggaggagtgtcactgtagattccatggacgaggagaaaattgaagaatatctgaagcgacagggtatttcttccatgcaggaatctggaccaaagaaagtggcccctattcagagaaggaaaaagcctgcttcacagaaaaagcgacgctttaagactcataacgaacacttggctggagtgctgaaggattactctgacattacttccagcaaatagggaacagttttgccctggaacagagttacagatacacaatcaagagtgttcttgctgatgctcggggtctgaagactgtcttcctatctgcttcttgcggctgaggagaggagcagttcagtttacaaaacaagtgcaaattaccaaactcaaagcttatttgagtagaatgggctcatgggcaatgtgatgttccctgttaaccttctgttactccctgggagaaaggcgctgagcgtggcatgcaggtgtctttgctgtgtttttctccacttctaaatggttcctggttcctttcttcctcgtttgttactttagagcaagtttgcccatagtcttgaatgcaatatttgtttattccaaaagaacatatttataataaaatcactgtagaaggatttttaagatgttagtgaattctgtttcttttcattctcggaaatggcaggaagcagctccagtctctgatttccatgggtcacgtgctggggatgtgatgaagcctgcagtctgcactgtgttgctgagcacatggatttcaccactggaacacaggtgtgctgcttgttagcaagcagagcaataaagatgtgctggatgtca

SEQ ID NO: 119 - DNA, 434 bp, Homo sapiens
actgagctcctagcacccgatcgggaagtggcgggcggagtcccgggtccagtcgccgcctcagctaccgccgctgccgccgccgccgccgccaccgccagtggtgagaccccgacctggcgggtcagcgctgggcgtgcgtgcgggcaggcgggggcgctgacgagaagcaggaagagggtgcagtgccggcgtgggcggccggccgaggcggaggcgcaggaagggggcggcgagtcgtgcgaggctgcccttctcactcagcattatggatccaagcctgttgagagaaagggagctgttcaaaaaacgagctctttctactcctgtagtagaaaaacgttcagcatcttctgagtcatcatcatcatcgtcaaagaagaagaaaacaaaggtagaacatggaggatcgtcaggctctaaacaaaattctg

SEQ ID NO: 120 - DNA, 150 bp, Homo sapiens
atcctaaacagcacatccaatcacctgtggctagagttcaacaccaatggatctgacaccgaccaaggttttcaactcacctataccactacatctacatccaccactgggacaagccatcttgtaaaatgtgcggagaaggagaaaact

SEQ ID NO: 121 - DNA, 583 bp, Homo sapiens
ctccacggcagcggctccttgtgccactagcagcccttcttctgcgctctccgccttttctctctagactggatctctcctcccccccgcgcccccctccccgcatctcccactcgctggctctctctccagctgcctcctctccaggtctctcctggctgcgcgcgctcctctccccgcttctccccctcccgcagcctcgccgccttggtgccttcctgcccggctcggccggcgctcgtccccggccccggccccgccagcccgggtctccgcgctcggagcagctcagccctgcagtggctcgggacccgatgctatgagagggaagcgagccgggcgcccagaccttcaggaggcgtcggatgcgcggcgggtcttgggaccgggctctctctccggctcgccttgccctcgggtgattatttggctccgctcatagccctgccttcctcggaggagccatcggtgtcgcgtgcgtgtggagtatctgcagacatgactgcgtggaggagattccagtcgctgctcctgcttctcgggctgctggtgctgtgcgcgaggctcctcactgcagcgaagg

SEQ ID NO: 122 - DNA, 217 bp, Homo sapiens
gtcagaactgtggaggcttagtccagggtcccaatggcactattgagagcccagggtttcctcacgggtatccgaactatgccaactgcacctggatcatcatcacgggcgagcgcaataggatacagttgtccttccatacctttgctcttgaagaagattttgatattttatcagtttacgatggacagcctcaacaagggaatttaaaagtgag

SEQ ID NO: 123 - DNA, 113 bp, Homo sapiens
attatcgggatttcagctgccctcctctatagtgagtacaggatctatcctcactctgtggttcacgacagacttcgctgtgagtgcccaaggtttcaaagcattatatgaag

SEQ ID NO: 124 - DNA, 195 bp, Homo sapiens
ttttacctagccacacttgtggaaatcctggagaaatcctgaaaggagttctgcatggaacgagattcaacataggagacaaaatccggtacagctgcctccctggctacatcttggaaggccacgccatcctgacctgcatcgtcagcccaggaaatggtgcatcgtgggacttcccagctcccttttgcagag

SEQ ID NO: 125 - DNA, 208 bp, Homo sapiens
ctgagggagcctgcggaggaaccttacgcgggaccagcagctccatctccagcccgcacttcccttcagagtacgagaacaacgcggactgcacctggaccattctggctgagcccggggacaccattgcgctggtcttcactgactttcagctagaagaaggatatgatttcttagagatcagtggcacggaagctccatccatatg

SEQ ID NO: 126 - DNA, 113 bp, Homo sapiens
gctaactggcatgaacctcccctctccagttatcagtagcaagaattggctacgactccatttcacctctgacagcaaccaccgacgcaaaggatttaacgctcagttccaag

SEQ ID NO: 127 - DNA, 78 bp, Homo sapiens
tgaaaaaggcgattgagttgaagtcaagaggagtcaagatgctgcccagcaaggatggaagccataaaaactctgtct

SEQ ID NO: 128 - DNA, 88 bp, Homo sapiens
tgagccaaggaggtgttgcattggtctctgacatgtgtccagatcctgggattccagaaaatggtagaagagcaggttccgacttcag

SEQ ID NO: 129 - DNA, 125 bp, Homo sapiens
ggttggtgcaaatgtacagttttcatgtgaggacaattacgtgctccagggatctaaaagcatcacctgtcagagagttacagagacgctcgctgcttggagtgaccacaggcccatctgccgag

SEQ ID NO: 130 - DNA, 122 bp, Homo sapiens
cgagaacatgtggatccaatctgcgtgggcccagcggcgtcattacctcccctaattatccggttcagtatgaagataatgcacactgtgtgtgggtcatcaccaccaccgacccggacaag

SEQ ID NO: 131 - DNA, 104 bp, Homo sapiens
gtcatcaagcttgcctttgaagagtttgagctggagcgaggctatgacaccctgacggttggtgatgctgggaaggtgggagacaccagatcggtcttgtacgt

SEQ ID NO: 132 - DNA, 113 bp, Homo sapiens
gctcacgggatccagtgttcctgacctcattgtgagcatgagcaaccagatgtggctacatctgcagtcggatgatagcattggctcacctgggtttaaagctgtttaccaag

SEQ ID NO: 133 - DNA, 183 bp, Homo sapiens
aaattgaaaagggagggtgtggggatcctggaatccccgcctatgggaagcggacgggcagcagtttcctccatggagatacactcacctttgaatgcccggcggcctttgagctggtgggggagagagttatcacctgtcagcagaacaatcagtggtctggcaacaagcccagctgtgtat

SEQ ID NO: 134 - DNA, 327 bp, Homo sapiens
tttcatgtttcttcaactttacggcatcatctgggattattctgtcaccaaattatccagaggaatatgggaacaacatgaactgtgtctggttgattatctcggagccaggaagtcgaattcacctaatctttaatgattttgatgttgagcctcagtttgactttctcgcggtcaaggatgatggcatttctgacataactgtcctgggtactttttctggcaatgaagtgccttcccagctggccagcagtgggcatatagttcgcttggaatttcagtctgaccattccactactggcagagggttcaacatcacttacacca

SEQ ID NO: 135 - DNA, 195 bp, Homo sapiens
catttggtcagaatgagtgccatgatcctggcattcctataaacggacgacgttttggtgacaggtttctactcgggagctcggtttctttccactgtgatgatggctttgtcaagacccagggatccgagtccattacctgcatactgcaagacgggaacgtggtctggagctccaccgtgccccgctgtgaag

SEQ ID NO: 136 - DNA, 139 bp, Homo sapiens
ctccatgtggtggacatctgacagcgtccagcggagtcattttgcctcctggatggccaggatattataaggattctttacattgtgaatggataattgaagcaaaaccaggccactctatcaaaataacttttgacag

SEQ ID NO: 137 - DNA, 188 bp, Homo sapiens
atttcagacagaggtcaattatgacaccttggaggtcagagatgggccagccagttcgtccccactgatcggcgagtaccacggcacccaggcaccccagttcctcatcagcaccgggaacttcatgtacctgctgttcaccactgacaacagccgctccagcatcggcttcctcatccactatgaga

SEQ ID NO: 138 - DNA, 189 bp, Homo sapiens
gtgtgacgcttgagtcggattcctgcctggacccgggcatccctgtgaacggccatcgccacggtggagactttggcatcaggtccacagtgactttcagctgtgacccggggtacacactaagtgacgacgagcccctcgtctgtgagaggaaccaccagtggaaccacgccttgcccagctgcgacg

SEQ ID NO: 139 - DNA, 117 bp, Homo sapiens
ctctatgtggaggctacatccaagggaagagtggaacagtcctttctcctgggtttccagatttttatccaaactctctaaactgcacgtggaccattgaagtgtctcatgggaaag

SEQ ID NO: 140 - DNA, 216 bp, Homo sapiens
gagttcaaatgatctttcacacctttcatcttgagagttcccacgactatttactgatcacagaggatggaagtttttccgagcccgttgccaggctcaccgggtcggtgttgcctcatacgatcaaggcaggcctgtttggaaacttcactgcccagcttcggtttatatcagacttctcaatttcgtacgagggcttcaatatcacattttcag

SEQ ID NO: 141 - DNA, 189 bp, Homo sapiens
aatatgacctggagccatgtgatgatcctggagtccctgccttcagccgaagaattggttttcactttggtgtgggagactctctgacgttttcctgcttcctgggatatcgtttagaaggtgccaccaagcttacctgcctgggtgggggccgccgtgtgtggagtgcacctctgccaaggtgtgtgg

SEQ ID NO: 142 - DNA, 170 bp, Homo sapiens
ccgaatgtggagcaagtgtcaaaggaaatgaaggaacattactgtctccaaattttccatccaattatgataataaccatgagtgtatctataaaatagaaacagaagccggcaagggcatccaccttagaacacgaagcttccagctgtttgaaggagatactctaaag

SEQ ID NO: 143 - DNA, 157 bp, Homo sapiens
gtatatgatggaaaagacagttcctcacgtccactgggcacgttcactaaaaatgaacttctggggctgatcctaaacagcacatccaatcacctgtggctagagttcaacaccaatggatctgacaccgaccaaggttttcaactcacctatacca

SEQ ID NO: 144 - DNA, 14417 bp, Homo sapiens
ctccacggcagcggctccttgtgccactagcagcccttcttctgcgctctccgccttttctctctagactggatctctcctcccccccgcgcccccctccccgcatctcccactcgctggctctctctccagctgcctcctctccaggtctctcctggctgcgcgcgctcctctccccgcttctccccctcccgcagcctcgccgccttggtgccttcctgcccggctcggccggcgctcgtccccggccccggccccgccagcccgggtctccgcgctcggagcagctcagccctgcagtggctcgggacccgatgctatgagagggaagcgagccgggcgcccagaccttcaggaggcgtcggatgcgcggcgggtcttgggaccgggctctctctccggctcgccttgccctcgggtgattatttggctccgctcatagccctgccttcctcggaggagccatcggtgtcgcgtgcgtgtggagtatctgcagacatgactgcgtggaggagattccagtcgctgctcctgcttctcgggctgctggtgctgtgcgcgaggctcctcactgcagcgaagggtcagaactgtggaggcttagtccagggtcccaatggcactattgagagcccagggtttcctcacgggtatccgaactatgccaactgcacctggatcatcatcacgggcgagcgcaataggatacagttgtccttccatacctttgctcttgaagaagattttgatattttatcagtttacgatggacagcctcaacaagggaatttaaaagtgagattatcgggatttcagctgccctcctctatagtgagtacaggatctatcctcactctgtggttcacgacagacttcgctgtgagtgcccaaggtttcaaagcattatatgaagttttacctagccacacttgtggaaatcctggagaaatcctgaaaggagttctgcatggaacgagattcaacataggagacaaaatccggtacagctgcctccctggctacatcttggaaggccacgccatcctgacctgcatcgtcagcccaggaaatggtgcatcgtgggacttcccagctcccttttgcagagctgagggagcctgcggaggaaccttacgcgggaccagcagctccatctccagcccgcacttcccttcagagtacgagaacaacgcggactgcacctggaccattctggctgagcccggggacaccattgcgctggtcttcactgactttcagctagaagaaggatatgatttcttagagatcagtggcacggaagctccatccatatggctaactggcatgaacctcccctctccagttatcagtagcaagaattggctacgactccatttcacctctgacagcaaccaccgacgcaaaggatttaacgctcagttccaagtgaaaaaggcgattgagttgaagtcaagaggagtcaagatgctgcccagcaaggatggaagccataaaaactctgtcttgagccaaggaggtgttgcattggtctctgacatgtgtccagatcctgggattccagaaaatggtagaagagcaggttccgacttcagggttggtgcaaatgtacagttttcatgtgaggacaattacgtgctccagggatctaaaagcatcacctgtcagagagttacagagacgctcgctgcttggagtgaccacaggcccatctgccgagcgagaacatgtggatccaatctgcgtgggcccagcggcgtcattacctcccctaattatccggttcagtatgaagataatgcacactgtgtgtgggtcatcaccaccaccgacccggacaaggtcatcaagcttgcctttgaagagtttgagctggagcgaggctatgacaccctgacggttggtgatgctgggaaggtgggagacaccagatcggtcttgtacgtgctcacgggatccagtgttcctgacctcattgtgagcatgagcaaccagatgtggctacatctgcagtcggatgatagcattggctcacctgggtttaaagctgtttaccaagaaattgaaaagggagggtgtggggatcctggaatccccgcctatgggaagcggacgggcagcagtttcctccatggagatacactcacctttgaatgcccggcggcctttgagctggtgggggagagagttatcacctgtcagcagaacaatcagtggtctggcaacaagcccagctgtgtattttcatgtttcttcaactttacggcatcatctgggattattctgtcaccaaattatccagaggaatatgggaacaacatgaactgtgtctggttgattatctcggagccaggaagtcgaattcacctaatctttaatgattttgatgttgagcctcagtttgactttctcgcggtcaaggatgatggcatttctgacataactgtcctgggtactttttctggcaatgaagtgccttcccagctggccagcagtgggcatatagttcgcttggaatttcagtctgaccattccactactggcagagggttcaacatcacttacaccacatttggtcagaatgagtgccatgatcctggcattcctataaacggacgacgttttggtgacaggtttctactcgggagctcggtttctttccactgtgatgatggctttgtcaagacccagggatccgagtccattacctgcatactgcaagacgggaacgtggtctggagctccaccgtgccccgctgtgaagctccatgtggtggacatctgacagcgtccagcggagtcattttgcctcctggatggccaggatattataaggattctttacattgtgaatggataattgaagcaaaaccaggccactctatcaaaataacttttgacagatttcagacagaggtcaattatgacaccttggaggtcagagatgggccagccagttcgtccccactgatcggcgagtaccacggcacccaggcaccccagttcctcatcagcaccgggaacttcatgtacctgctgttcaccactgacaacagccgctccagcatcggcttcctcatccactatgagagtgtgacgcttgagtcggattcctgcctggacccgggcatccctgtgaacggccatcgccacggtggagactttggcatcaggtccacagtgactttcagctgtgacccggggtacacactaagtgacgacgagcccctcgtctgtgagaggaaccaccagtggaaccacgccttgcccagctgcgacgctctatgtggaggctacatccaagggaagagtggaacagtcctttctcctgggtttccagatttttatccaaactctctaaactgcacgtggaccattgaagtgtctcatgggaaaggagttcaaatgatctttcacacctttcatcttgagagttcccacgactatttactgatcacagaggatggaagtttttccgagcccgttgccaggctcaccgggtcggtgttgcctcatacgatcaaggcaggcctgtttggaaacttcactgcccagcttcggtttatatcagacttctcaatttcgtacgagggcttcaatatcacattttcagaatatgacctggagccatgtgatgatcctggagtccctgccttcagccgaagaattggttttcactttggtgtgggagactctctgacgttttcctgcttcctgggatatcgtttagaaggtgccaccaagcttacctgcctgggtgggggccgccgtgtgtggagtgcacctctgccaaggtgtgtggccgaatgtggagcaagtgtcaaaggaaatgaaggaacattactgtctccaaattttccatccaattatgataataaccatgagtgtatctataaaatagaaacagaagccggcaagggcatccaccttagaacacgaagcttccagctgtttgaaggagatactctaaaggtatatgatggaaaagacagttcctcacgtccactgggcacgttcactaaaaatgaacttctggggctgatcctaaacagcacatccaatcacctgtggctagagttcaacaccaatggatctgacaccgaccaaggttttcaactcacctataccagttttgatctggtaaaatgtgaggatccgggcatccctaactacggctataggatccgtgatgaaggccactttaccgacactgtagttctgtacagttgcaacccggggtacgccatgcatggcagcaacaccctgacctgtttgagtggagacaggagagtgtgggacaaaccactaccttcgtgcatagcggaatgtggtggtcagatccatgcagccacatcaggacgaatattgtcccctggctatccagctccgtatgacaacaacctccactgcacctggattatagaggcagacccaggaaagaccattagcctccatttcattgttttcgacacggagatggctcacgacatcctcaaggtctgggacgggccggtggacagtgacatcctgctgaaggagtggagtggctccgcccttccggaggacatccacagcaccttcaactcactcaccctgcagttcgacagcgacttcttcatcagcaagtctggcttctccatccagttctccacctcaattgcagccacctgtaacgatccaggtatgccccaaaatggcacccgctatggagacagcagagaggctggagacaccgtcacattccagtgtgaccctggctatcagctccaaggacaagccaaaatcacctgtgtgcagctgaataaccggttcttttggcaaccagaccctcctacatgcatagctgcttgtggagggaatctgacgggcccagcaggtgttattttgtcacccaactacccacagccgtatcctcctgggaaggaatgtgactggagagtaaaagtgaacccggactttgtcatcgccttgatattcaaaagtttcaacatggagcccagctatgacttcctacacatctatgaaggggaagattccaacagccccctcattgggagttaccagggctctcaggccccagaaagaatagagagtagcggaaacagcctgtttctggcatttcggagtgatgcctccgtgggcctttcagggttcgccattgaatttaaagagaaaccacgggaagcttgttttgacccaggaaatataatgaatgggacaagagttggaacagacttcaagcttggctccaccatcacctaccagtgtgactctggctataagattcttgacccctcatccatcacctgtgtgattggggctgatgggaaaccctcctgggaccaagtgctgccctcctgcaatgctccctgtggaggccagtacacgggatcagaaggggtagttttatcaccaaactacccccataattacacagctggtcaaatatgcctctattccatcacggtaccaaaggaattcgtggtctttggacagtttgcctatttccagacagccctgaatgatttggcagaattatttgatggaacccatgcacaggccagacttctcagctcactctcggggtctcactcaggggaaacattgcccttggctacgtcaaatcaaattctgctccgattcagtgcaaagagcggtgcctctgcccgcggcttccacttcgtgtatcaagctgttcctcgtaccagtgacacccaatgcagctctgtccccgagcccagatacggaaggagaattggttctgagttttctgccggctccatcgtccgattcgagtgcaacccgggatacctgcttcagggttccacggcgctccactgccagtccgtgcccaacgccttggcacagtggaacgacacgatccccagctgtgtggtaccctgcagtggcaatttcactcaacgaagaggtacaatcctgtcccccggctaccctgagccatacggaaacaacttgaactgtatatggaagatcatagttacggagggctcgggaattcagatccaagtgatcagttttgccacggagcagaactgggactcccttgagatccacgatggtggggatgtgaccgcacccagactgggaagcttctcaggcaccacagtaccggcactgctgaacagtacttccaaccaactctacctgcatttccagtctgacattagtgtggcagctgctggtttccacctggaatacaaaactgtaggtcttgctgcatgccaagaaccagccctccccagcaacagcatcaaaatcggagatcggtacatggtgaacgacgtgctctccttccagtgcgagcccgggtacaccctgcagggccgttcccacatttcctgtatgccagggaccgttcgccgttggaactatccgtctcccctgtgcattgcaacctgtggagggacgctgagcaccttgggtggtgtgatcctgagccccggcttcccaggttcttaccccaacaacttagactgcacctggaggatctcattacccatcggctatggtgcacatattcagtttctgaatttttctaccgaagctaatcatgacttccttgaaattcaaaatggaccttaccacaccagccccatgattggacaatttagcggcacggatctccccgcggccctgctgagcacaacgcatgaaaccctcatccacttttatagtgaccattcgcaaaaccggcaaggatttaaacttgcttaccaagcctatgaattacagaactgtccagatccacccccatttcagaatgggtacatgatcaactcggattacagcgtggggcaatcagtatctttcgagtgttatcctgggtacattctaataggccatcctgtcctcacttgtcagcatgggatcaacagaaactggaactacccttttccaagatgtgatgccccttgtgggtacaacgtaacttctcagaacggcaccatctactcccctggctttcctgatgagtatccgatcctgaaggactgcatttggctcatcacggtgcctccagggcacggagtttacatcaacttcaccctgttacagacggaagctgtcaacgattacattgctgtttgggacggtcccgatcagaactcaccccagctgggagttttcagtggcaacacagccctcgaaacggcgtatagctccaccaaccaagtcctgctcaagttccacagcgacttttcaaatggaggcttctttgtcctcaatttccacgcatttcagctcaagaaatgtcaacctcccccagcggttccacaggcagaaatgcttactgaggatgatgatttcgaaataggagattttgtgaagtaccagtgccaccccgggtacaccttggtggggaccgacattctgacttgcaagctcagttcccagttgcagtttgagggttctctcccaacatgtgaagcacaatgcccagcaaatgaagtccggactggatcatcgggagtcattctcagtccagggtatccgggtaattattttaactcccagacttgctcttggagtattaaagtggaaccaaactacaacattaccatctttgtggacacatttcaaagtgaaaagcagtttgatgcactggaagtgtttgatggttcttctgggcaaagtcctctgctagtagtcttaagtgggaatcatactgaacaatcaaattttacaagcaggagtaatcagttatatctccgctggtccactgaccatgccaccagtaagaaaggattcaagattcgctatgcagcaccttactgcagtttgacccaccccctgaagaatgggggtattctaaacaggactgcaggagcggttggaagcaaagtgcattatttttgcaagcctggataccgaatggtcggccacagcaatgcaacctgtagacgaaacccacttggcatgtaccagtgggactccctcacgccactctgccaggctgtgtcctgtggaatcccagaatccccaggaaacggttcatttaccgggaacgagttcactttggacagtaaagtggtctatgaatgtcatgagggcttcaagcttgaatccagccagcaagcaacagccgtgtgtcaagaagatgggttgtggagtaacaaggggaagccgcccacgtgtaagccggtcgcttgccccagcattgaagctcagctctcagaacatgtcatctggaggctggtttcaggatccttgaatgagtacggtgctcaagtattgctgagctgcagtcctggttactacttagaaggctggaggctcctgcggtgccaggccaatgggacgtggaacataggagatgagaggccaagctgtcgagttatctcgtgtggaagcctttcctttcccccaaatggcaacaagattggaacgttgacagtttatggggccacagctatatttacgtgcaacaccggctacacgcttgtggggtctcatgtcagagagtgcttggcaaatgggctctggagcggcagcgaaactcgatgtctggctggccactgcggttccccagacccgattgtgaacggtcacattagtggagatggcttcagttacagagacacggtggtttaccagtgcaatcctggtttccggcttgtgggaacttccgtgaggatatgcctgcaagaccacaagtggtctggacaaacgcctgtctgtgtccccatcacatgtggtcaccctggaaaccctgcccacggattcactaatggcagtgagttcaacctgaatgatgtcgtgaatttcacctgcaacacgggctatttgctgcagggcgtgtctcgagcccagtgtcggagcaacggccagtggagtagccctctgcccacgtgtcgagtggtgaactgttctgatccaggctttgtggaaaatgccattcgtcacgggcaacagaacttccctgagagttttgagtatggaatgagtatcctgtaccattgcaagaagggattttacttgctgggatcttcagccttgacctgtatggcaaatggcttatgggaccgatccctgcccaagtgtttggctatatcgtgtggacacccaggggtccctgccaacgccgtcctcactggagagctgtttacctatggcgccgtcgtgcactactcctgcagagggagcgagagcctcataggcaacgacacgagagtgtgccaggaagacagtcactggagcggggcactgccccactgcacaggaaataatcctggattctgtggtgatccggggaccccagcacatgggtctcggcttggtgatgactttaagacaaagagtcttctccgcttctcctgtgaaatggggcaccagctgaggggctcccctgaacgcacgtgtttgctcaatgggtcatggtcaggactgcagccggtgtgtgaggccgtgtcctgtggcaaccctggcacacccaccaacggaatgattgtcagtagtgatggcattctgttctccagctcggtcatctatgcctgctgggaaggctacaagacctcagggctcatgacacggcattgcacagccaatgggacctggacaggcactgctcccgactgcacaattataagttgtggggatccaggcacactagcaaatggcatccagtttgggaccgacttcaccttcaacaagactgtgagctatcagtgtaacccaggctatgtcatggaagcagtcacatccgccactattcgctgtaccaaagacggcaggtggaatccgagcaaacctgtctgcaaagccgtgctgtgtcctcagccgccgccggtgcagaatggaacagtggagggaagtgatttccgctggggctccagcataagttacagctgcatggacggttaccagctctctcactccgccatcctctcctgtgaaggtcgcggggtgtggaaaggagagatcccccagtgtctccctgtgttctgcggagaccctggcatccccgcagaagggcgacttagtgggaaaagtttcacctataagtccgaagtcttcttccagtgcaaatctccatttatactcgtgggatcctccagaagagtctgccaagctgacggcacgtggagcggcatacaacccacctgcattgatcctgctcataacacctgcccagaccctggtacgccacactttggaatacagaatagctccagaggctatgaggttggaagcacggtttttttcaggtgcagaaaaggctaccatattcaaggttccacgactcgcacctgccttgccaatttaacatggagtgggatacagaccgaatgtatacctcatgcctgcagacagccagaaaccccggcacacgcggatgtgagagccatcgatcttcctactttcggctacaccttagtgtacacctgccatccaggctttttcctcgcagggggatctgagcacagaacatgtaaagcagacatgaaatggacaggaaagtcgcctgtgtgtaaaagtaaaggagtgagagaagttaatgaaacagttactaaaactccagttccttcagatgtctttttcgtcaattcactgtggaaggggtattatgaatatttagggaaaagacaacccgccactctaactgttgactggttcaatgcaacaagcagtaaggtgaatgccaccttcagcgaagcctcgccagtggagctgaagttgacaggcatttacaagaaggaggaggcccacttactcctgaaagcttttcaaattaaaggccaggcagatatttttgtaagcaagttcgaaaatgacaactggggactagatggttatgtgtcatctggacttgaaagaggaggatttacttttcaaggtgacattcatggaaaagactttggaaaatttaagctagaaaggcaagatcctttaaacccagatcaagactcttccagtcattaccacggcaccagcagtggctctgtggcggctgccattctggttcctttctttgctctaattttatcagggtttgcattttacctctacaaacacagaacgagaccaaaagttcaatacaatggctatgctgggcatgaaaacagcaatggacaagcatcgtttgaaaaccccatgtatgatacaaacttaaaacccacagaagccaaggctgtgaggtttgacacaactctgaacacagtctgtacagtggtatagccctcagtgccccaacaggactgattcatagccatacctctgatggacaagcagtgattcctttggtgccatataccactctcccttccactctggctttactgcagcgatcttcaaccttgtctactggcataagtgcagcggggatctctactcaaatgtgtcagggtcttctacggatcaaactacacatgcgttttcattccaaaagtgggttctaaatgcctggctgcatctgtatgaaatcaaggcacactccaggaagactgccacgtcgcgccaacacgtcatactcaatgcctcagactttcatatttctgtgttgctgagatgcctttcaatgcaatcgtctgggctcgtggatatgtccctcaggtgcggtgacagaatggtggcaccacgatatgtgttctcttgtgttgtttttcctttttaaacccccatgaacacgaatactctgaaaaaaataaaaagctttctggaagaagacacctttctgatagaggctcacacctacaaatgcttcactctgtccttccgagacctgacaagctttgaggacctcacagctcccctgtgtgttcatctctagggatgtttgcaatttcccagtcagctgttctgtcgcagaatgtttaatgcacaattttttgcactagtgtgttatgaatgactaagattctgataaaaaaaataaattatttacacagggtttatacacactatccattgtatataagcattatttcatattatcaagctaaacattcccccatcagcttagttggagtgttagggaaaagtattcctagatatggcacagattttaaaaggaaatacagtattgaagagatttattttattattgcttcaattagctccatttacgtgttgaattcattgaagaggtccaatgagaaaaaaacagaagcctccttatttcacacgttttcctcctttagtaccatcctcatccaattactgtctctctgatactacttaatagcagggggtttgcagaaatttctgtttgccatgtaaaactgtgaatagtaatttattttagatagtcgatgaacttgtgggttttagctcacaatgcagccttcccttttgcagtgttttttttttgtttttttttttttttgtcttttactgtgccatcgatctttgatattgcattgaaagacaatataccacagtagcaccttgaactcagtgaaaattgttcaggatcaaaataccaagtgttcttttagagggaaggaaaaagtacacacactctcctctcacaatgatatattttatacattcatttgttatttgtttcatgctttatgattccagatggaaaggtaatttcagtgacttttcaagtttaaattccattataggtaaatgataagttatgatgcaaataaaatctataagatccccagggcaaataaaaatcaaaacatgaagtagaagatgtggccgtgaggtagtttatgtaacaaattcaaagtgaaaatcatgtttacttttacttatacttatttgataaaaatatttttgaaacgatagtacttattttattatttgatatttcagttcctattcaattgtggcagattttctctgtttcacattttagattggcgttggtaatagaaatgtcagaatgttcaaattggccttcacgttgtcggagtgaacacattgacacctagctttaagactgatttatctgttggtgtactgaaggtttccatgtaggacttcaaatgtggaaaaggaaaagcagtcaggaaaatggggcattctttggagagtcacgcgttttgattcggacatttccgtagagctcggctcccagtgttgtgttcctcggtcgaaagggtctctgctgtttggggactcactggcctctcctagggactcctttgtcttgtgaaccccacgctgttggattctgtatcattatgctgaattctctgcacagttttccctggccaacctgcccacatccttggagatttgctttgccagtgggaatccttacattgctgtttcacagtagacgggacgaggtcagcgggagtcgtgctcctaacacacacattgaacgaaacagaagatgattgaaagtgtgaggaggctcgtgtgcaagggagaacagggttactatacatattagtgtatatatatacatacatatatatatatatatattgtacatatctaagtttgagtcattcaaactaggtgcaaaatgctgacttcagagtctgaattaacatctctgttcccatatccctgacctgctccctggtcaacgatgctatgaaatcctgaaatgacaggacatacatacatacaagaaaccacatatcaaattagatatgattttcctttgtgtgcaaagtcaaactgtcctagggttgccagtttgaagcatgttatttaaatgaaaaaaaaaatcagtgaaattctcgtgtgagaattctgcctagtttcttcctaaggttgtgtgcagtgttgaacggcgtctccgcaaggtgttggaggatctcattttagggcagtcaggagctgtgcttgctgagttaggtctagaagactcttccctgaaggcaacgggaacacgcgtgagggacgcgaccacacactaacagaggacacgtgcttcagagctgtttaaaactgctgcttgttttacacacacatcttgccttttttcaggctagctgcaataatttttttcttctgtaaaatattttgtaaacaacaacaaaaagctattataaaaagggggtaaaaaaaagaacgctggcattatgatcaggaaaacccattgtcatcgccgaccctccctcccgtcccaccacacgctgctgtcacgacgtaggtgcgaaagacctttttgtacagagatatattttttatgaagaatttgtaaaattattaaatatgctgtaattttttgattaatgtaggtacattgttaaaaaataaatgtttttacaatacagaactgtaattttcccaataatgtaaaatgtaccatctctagctgattttcagttccaatcctattacacatgtattaatattaaagtggcctgttaaaatgaacagtatcttttttttgtcaaaaaaattataaagagggtgtaatatagcctgtgcaatgccaccaatctttaaagcaaatcagagttctaattaaatatttaatttta

SEQ ID NO: 145 - DNA, 4129 bp, Homo sapiens
ctccacggcagcggctccttgtgccactagcagcccttcttctgcgctctccgccttttctctctagactggatctctcctcccccccgcgcccccctccccgcatctcccactcgctggctctctctccagctgcctcctctccaggtctctcctggctgcgcgcgctcctctccccgcttctccccctcccgcagcctcgccgccttggtgccttcctgcccggctcggccggcgctcgtccccggccccggccccgccagcccgggtctccgcgctcggagcagctcagccctgcagtggctcgggacccgatgctatgagagggaagcgagccgggcgcccagaccttcaggaggcgtcggatgcgcggcgggtcttgggaccgggctctctctccggctcgccttgccctcgggtgattatttggctccgctcatagccctgccttcctcggaggagccatcggtgtcgcgtgcgtgtggagtatctgcagacatgactgcgtggaggagattccagtcgctgctcctgcttctcgggctgctggtgctgtgcgcgaggctcctcactgcagcgaagggtcagaactgtggaggcttagtccagggtcccaatggcactattgagagcccagggtttcctcacgggtatccgaactatgccaactgcacctggatcatcatcacgggcgagcgcaataggatacagttgtccttccatacctttgctcttgaagaagattttgatattttatcagtttacgatggacagcctcaacaagggaatttaaaagtgagattatcgggatttcagctgccctcctctatagtgagtacaggatctatcctcactctgtggttcacgacagacttcgctgtgagtgcccaaggtttcaaagcattatatgaagttttacctagccacacttgtggaaatcctggagaaatcctgaaaggagttctgcatggaacgagattcaacataggagacaaaatccggtacagctgcctccctggctacatcttggaaggccacgccatcctgacctgcatcgtcagcccaggaaatggtgcatcgtgggacttcccagctcccttttgcagagctgagggagcctgcggaggaaccttacgcgggaccagcagctccatctccagcccgcacttcccttcagagtacgagaacaacgcggactgcacctggaccattctggctgagcccggggacaccattgcgctggtcttcactgactttcagctagaagaaggatatgatttcttagagatcagtggcacggaagctccatccatatggctaactggcatgaacctcccctctccagttatcagtagcaagaattggctacgactccatttcacctctgacagcaaccaccgacgcaaaggatttaacgctcagttccaagtgaaaaaggcgattgagttgaagtcaagaggagtcaagatgctgcccagcaaggatggaagccataaaaactctgtcttgagccaaggaggtgttgcattggtctctgacatgtgtccagatcctgggattccagaaaatggtagaagagcaggttccgacttcagggttggtgcaaatgtacagttttcatgtgaggacaattacgtgctccagggatctaaaagcatcacctgtcagagagttacagagacgctcgctgcttggagtgaccacaggcccatctgccgagcgagaacatgtggatccaatctgcgtgggcccagcggcgtcattacctcccctaattatccggttcagtatgaagataatgcacactgtgtgtgggtcatcaccaccaccgacccggacaaggtcatcaagcttgcctttgaagagtttgagctggagcgaggctatgacaccctgacggttggtgatgctgggaaggtgggagacaccagatcggtcttgtacgtgctcacgggatccagtgttcctgacctcattgtgagcatgagcaaccagatgtggctacatctgcagtcggatgatagcattggctcacctgggtttaaagctgtttaccaagaaattgaaaagggagggtgtggggatcctggaatccccgcctatgggaagcggacgggcagcagtttcctccatggagatacactcacctttgaatgcccggcggcctttgagctggtgggggagagagttatcacctgtcagcagaacaatcagtggtctggcaacaagcccagctgtgtattttcatgtttcttcaactttacggcatcatctgggattattctgtcaccaaattatccagaggaatatgggaacaacatgaactgtgtctggttgattatctcggagccaggaagtcgaattcacctaatctttaatgattttgatgttgagcctcagtttgactttctcgcggtcaaggatgatggcatttctgacataactgtcctgggtactttttctggcaatgaagtgccttcccagctggccagcagtgggcatatagttcgcttggaatttcagtctgaccattccactactggcagagggttcaacatcacttacaccacatttggtcagaatgagtgccatgatcctggcattcctataaacggacgacgttttggtgacaggtttctactcgggagctcggtttctttccactgtgatgatggctttgtcaagacccagggatccgagtccattacctgcatactgcaagacgggaacgtggtctggagctccaccgtgccccgctgtgaagctccatgtggtggacatctgacagcgtccagcggagtcattttgcctcctggatggccaggatattataaggattctttacattgtgaatggataattgaagcaaaaccaggccactctatcaaaataacttttgacagatttcagacagaggtcaattatgacaccttggaggtcagagatgggccagccagttcgtccccactgatcggcgagtaccacggcacccaggcaccccagttcctcatcagcaccgggaacttcatgtacctgctgttcaccactgacaacagccgctccagcatcggcttcctcatccactatgagagtgtgacgcttgagtcggattcctgcctggacccgggcatccctgtgaacggccatcgccacggtggagactttggcatcaggtccacagtgactttcagctgtgacccggggtacacactaagtgacgacgagcccctcgtctgtgagaggaaccaccagtggaaccacgccttgcccagctgcgacgctctatgtggaggctacatccaagggaagagtggaacagtcctttctcctgggtttccagatttttatccaaactctctaaactgcacgtggaccattgaagtgtctcatgggaaaggagttcaaatgatctttcacacctttcatcttgagagttcccacgactatttactgatcacagaggatggaagtttttccgagcccgttgccaggctcaccgggtcggtgttgcctcatacgatcaaggcaggcctgtttggaaacttcactgcccagcttcggtttatatcagacttctcaatttcgtacgagggcttcaatatcacattttcagaatatgacctggagccatgtgatgatcctggagtccctgccttcagccgaagaattggttttcactttggtgtgggagactctctgacgttttcctgcttcctgggatatcgtttagaaggtgccaccaagcttacctgcctgggtgggggccgccgtgtgtggagtgcacctctgccaaggtgtgtggccgaatgtggagcaagtgtcaaaggaaatgaaggaacattactgtctccaaattttccatccaattatgataataaccatgagtgtatctataaaatagaaacagaagccggcaagggcatccaccttagaacacgaagcttccagctgtttgaaggagatactctaaaggtatatgatggaaaagacagttcctcacgtccactgggcacgttcactaaaaatgaacttctggggctgatcctaaacagcacatccaatcacctgtggctagagttcaacaccaatggatctgacaccgaccaaggttttcaactcacctatacca

SEQ ID NO: 146 - DNA, 205 bp, Homo sapiens
ccagaactggaagtctgaagcgagccctgcacaatgccgaatgccagaagactgtcaccatctccaagccctgtggcaaactgaccaagcccaaacctcaagccttgcctccccgattgaaagagatgaaaagccaggaatcggctgcaggttccaaactagtccttcggtgtgaaaccagttctgaatactcctctctcagatt

SEQ ID NO: 147 - DNA, 532 bp, Homo sapiens
aaggggaaataagggacaagagagaccctctcatattgttttatattatttcatactcagaaaaggaaagagaagccaaacaaaaggcaggtaacccagcgcctaggaaccagacccgaaaccaaggaaccagatctgaaaccaggcctgggcctgcctgacctaagcctggtagtaaaaattccacccctgacctgacctggcaactgttgttatctacagattccagacattgtatggaaggacactgtgaaacctcccgttctgttctgtttcactctgaccatcggtgctcacagcccctatcacgtaccccctggcttgctcagtcgatcacgaccctctcacgtggacccccttagagttgttagcccttaaaagggacagaagttgagcacctgaggagctcagattttaagacgctaggctgctgatgctcccagctgattaaagccactcccttcactatctcggtgtctcctgtccgcggctcgtcctgctacatttcttggttccctgaccggcaagcgag

SEQ ID NO: 148 - DNA, 116 bp, Homo sapiens
aatgcaggctcaacagtaccagcagcagcgtcgaaaatttgcagctgccttcttggcattcattttcatactggcagctgtggatactgctgaagcagggaagaaagagaaaccag

SEQ ID NO: 149 - DNA, 174 bp, Homo sapiens
aaaaaaaagtgaagaagtctgactgtggagaatggcagtggagtgtgtgtgtgcccaccagtggagactgtgggctgggcacacgggagggcactcggactggagctgagtgcaagcaaaccatgaagacccagagatgtaagatcccctgcaactggaagaagcaatttggcg

SEQ ID NO: 150 - DNA, 162 bp, Homo sapiens
cggagtgcaaataccagttccaggcctggggagaatgtgacctgaacacagccctgaagaccagaactggaagtctgaagcgagccctgcacaatgccgaatgccagaagactgtcaccatctccaagccctgtggcaaactgaccaagcccaaacctcaag

SEQ ID NO: 151 - DNA, 756 bp, Homo sapiens
cagaatctaagaagaagaaaaaggaaggcaagaaacaggagaagatgctggattaaaagatgtcacctgtggaacataaaaaggacatcagcaaacaggatcagttaactattgcatttatatgtaccgtaggctttgtattcaaaaattatctatagctaagtacacaataagcaaaaacaaaaagaaaagaaaatttttgtagtagcgttttttaaatgtatactatagtaccagtaggggcttataataaaggactgtaatcttatttaggaagttgacttatagtacatgataaatgatagacaattgaggtaagttttttgaaattatgtgacattttacattaaattttttttacattttttgggcagcaatttaaatgttatgactatgtaaactacttctcttgttaggtaatttttttcacctagatttttttcccaattgagaaaaatatatactaaacaaaatagcaataaaacataatcactctatttgaagaaaatatcttgttttctgccaatagattttttaaaatgtagtcagcaaaatgggggtggggaagcagagcatgtcctagttcaatgttgactttttttttttttaaagaaaagcattaagacataaaattctttcactttggcagaagcatttgttttcttgatgaaattatttttccatctgaggaaaaaaatactaggaaaataaatcaaggtgatgctgaaaaaaaaaaaaaaaaaaaaaaaaaaaaaa

SEQ ID NO: 152 - DNA, 1740 bp, Homo sapiens
aaggggaaataagggacaagagagaccctctcatattgttttatattatttcatactcagaaaaggaaagagaagccaaacaaaaggcaggtaacccagcgcctaggaaccagacccgaaaccaaggaaccagatctgaaaccaggcctgggcctgcctgacctaagcctggtagtaaaaattccacccctgacctgacctggcaactgttgttatctacagattccagacattgtatggaaggacactgtgaaacctcccgttctgttctgtttcactctgaccatcggtgctcacagcccctatcacgtaccccctggcttgctcagtcgatcacgaccctctcacgtggacccccttagagttgttagcccttaaaagggacagaagttgagcacctgaggagctcagattttaagacgctaggctgctgatgctcccagctgattaaagccactcccttcactatctcggtgtctcctgtccgcggctcgtcctgctacatttcttggttccctgaccggcaagcgagaatgcaggctcaacagtaccagcagcagcgtcgaaaatttgcagctgccttcttggcattcattttcatactggcagctgtggatactgctgaagcagggaagaaagagaaaccagaaaaaaaagtgaagaagtctgactgtggagaatggcagtggagtgtgtgtgtgcccaccagtggagactgtgggctgggcacacgggagggcactcggactggagctgagtgcaagcaaaccatgaagacccagagatgtaagatcccctgcaactggaagaagcaatttggcgcggagtgcaaataccagttccaggcctggggagaatgtgacctgaacacagccctgaagaccagaactggaagtctgaagcgagccctgcacaatgccgaatgccagaagactgtcaccatctccaagccctgtggcaaactgaccaagcccaaacctcaagcagaatctaagaagaagaaaaaggaaggcaagaaacaggagaagatgctggattaaaagatgtcacctgtggaacataaaaaggacatcagcaaacaggatcagttaactattgcatttatatgtaccgtaggctttgtattcaaaaattatctatagctaagtacacaataagcaaaaacaaaaagaaaagaaaatttttgtagtagcgttttttaaatgtatactatagtaccagtaggggcttataataaaggactgtaatcttatttaggaagttgacttatagtacatgataaatgatagacaattgaggtaagttttttgaaattatgtgacattttacattaaattttttttacattttttgggcagcaatttaaatgttatgactatgtaaactacttctcttgttaggtaatttttttcacctagatttttttcccaattgagaaaaatatatactaaacaaaatagcaataaaacataatcactctatttgaagaaaatatcttgttttctgccaatagattttttaaaatgtagtcagcaaaatgggggtggggaagcagagcatgtcctagttcaatgttgactttttttttttttaaagaaaagcattaagacataaaattctttcactttggcagaagcatttgttttcttgatgaaattatttttccatctgaggaaaaaaatactaggaaaataaatcaaggtgatgctgaaaaaaaaaaaaaaaaaaaaaaaaaaaaaa

SEQ ID NO: 153 - DNA, 984 bp, Homo sapiens
aaggggaaataagggacaagagagaccctctcatattgttttatattatttcatactcagaaaaggaaagagaagccaaacaaaaggcaggtaacccagcgcctaggaaccagacccgaaaccaaggaaccagatctgaaaccaggcctgggcctgcctgacctaagcctggtagtaaaaattccacccctgacctgacctggcaactgttgttatctacagattccagacattgtatggaaggacactgtgaaacctcccgttctgttctgtttcactctgaccatcggtgctcacagcccctatcacgtaccccctggcttgctcagtcgatcacgaccctctcacgtggacccccttagagttgttagcccttaaaagggacagaagttgagcacctgaggagctcagattttaagacgctaggctgctgatgctcccagctgattaaagccactcccttcactatctcggtgtctcctgtccgcggctcgtcctgctacatttcttggttccctgaccggcaagcgagaatgcaggctcaacagtaccagcagcagcgtcgaaaatttgcagctgccttcttggcattcattttcatactggcagctgtggatactgctgaagcagggaagaaagagaaaccagaaaaaaaagtgaagaagtctgactgtggagaatggcagtggagtgtgtgtgtgcccaccagtggagactgtgggctgggcacacgggagggcactcggactggagctgagtgcaagcaaaccatgaagacccagagatgtaagatcccctgcaactggaagaagcaatttggcgcggagtgcaaataccagttccaggcctggggagaatgtgacctgaacacagccctgaagaccagaactggaagtctgaagcgagccctgcacaatgccgaatgccagaagactgtcaccatctccaagccctgtggcaaactgaccaagcccaaacctcaag

SEQ ID NO: 154 - DNA, 182 bp, Homo sapiens
caacagcaacaagatcacagttcgcttccactcagatcagtcctacaccgacaccggcttcttagctgaatacctctcctacgactccagtgaccctacatctacatccaccactgggacaagccatcttgtaaaatgtgcggagaaggagaaaactttctgtgtgaatggaggggagtgct

SEQ ID NO: 155 - DNA, 279 bp, Homo sapiens
gtgagagcggagctgcagccggagaaagaggaagagggagagagagcgcgccagggcgagggcaccgccgccggtcgggcgcgctgggcctgcccggaatcccgccgcctgcgccccgcgccccgcgccctgcgggccatgggagccggccgccggcagggacgacgcctgtgagacccgcgagcggcctcggggaccatggggagcgatcgggcccgcaagggcggagggggcccgaaggacttcggcgcgggactcaagtacaactcccggcacgag

SEQ ID NO: 156 - DNA, 160 bp, Homo sapiens
aaagtgaatggcttggaggaaggcgtggagttcctgccagtcaacaacgtcaagaaggtggaaaagcatggcccggggcgctgggtggtgctggcagccgtgctgatcggcctcctcttggtcttgctggggatcggcttcctggtgtggcatttgcagt

SEQ ID NO: 157 - DNA, 128 bp, Homo sapiens
accgggacgtgcgtgtccagaaggtcttcaatggctacatgaggatcacaaatgagaattttgtggatgcctacgagaactccaactccactgagtttgtaagcctggccagcaaggtgaaggacgcg

SEQ ID NO: 158 - DNA, 71 bp, Homo sapiens
ctgaagctgctgtacagcggagtcccattcctgggcccctaccacaaggagtcggctgtgacggccttcag

SEQ ID NO: 159 - DNA, 158 bp, Homo sapiens
cgagggcagcgtcatcgcctactactggtctgagttcagcatcccgcagcacctggtggaggaggccgagcgcgtcatggccgaggagcgcgtagtcatgctgcccccgcgggcgcgctccctgaagtcctttgtggtcacctcagtggtggctttcc

SEQ ID NO: 160 - DNA, 36 bp, Homo sapiens
ccacggactccaaaacagtacagaggacccaggaca

SEQ ID NO: 161 - DNA, 241 bp, Homo sapiens
acagctgcagctttggcctgcacgcccgcggtgtggagctgatgcgcttcaccacgcccggcttccctgacagcccctaccccgctcatgcccgctgccagtgggccctgcggggggacgccgactcagtgctgagcctcaccttccgcagctttgaccttgcgtcctgcgacgagcgcggcagcgacctggtgacggtgtacaacaccctgagccccatggagccccacgccctggtgca

SEQ ID NO: 162 - DNA, 140 bp, Homo sapiens
gttgtgtggcacctaccctccctcctacaacctgaccttccactcctcccagaacgtcctgctcatcacactgataaccaacactgagcggcggcatcccggctttgaggccaccttcttccagctgcctaggatgagca

SEQ ID NO: 163 - DNA, 98 bp, Homo sapiens
gctgtggaggccgcttacgtaaagcccaggggacattcaacagcccctactacccaggccactacccacccaacattgactgcacatggaacattgag

SEQ ID NO: 164 - DNA, 110 bp, Homo sapiens
gtgcccaacaaccagcatgtgaaggtgcgcttcaaattcttctacctgctggagcccggcgtgcctgcgggcacctgccccaaggactacgtggagatcaacggggagaa

SEQ ID NO: 165 - DNA, 131 bp, Homo sapiens
atactgcggagagaggtcccagttcgtcgtcaccagcaacagcaacaagatcacagttcgcttccactcagatcagtcctacaccgacaccggcttcttagctgaatacctctcctacgactccagtgacc

SEQ ID NO: 166 - DNA, 105 bp, Homo sapiens
catgcccggggcagttcacgtgccgcacggggcggtgtatccggaaggagctgcgctgtgatggctgggccgactgcaccgaccacagcgatgagctcaactgca

SEQ ID NO: 167 - DNA, 111 bp, Homo sapiens
gttgcgacgccggccaccagttcacgtgcaagaacaagttctgcaagcccctcttctgggtctgcgacagtgtgaacgactgcggagacaacagcgacgagcaggggtgca

SEQ ID NO: 168 - DNA, 114 bp, Homo sapiens
gttgtccggcccagaccttcaggtgttccaatgggaagtgcctctcgaaaagccagcagtgcaatgggaaggacgactgtggggacgggtccgacgaggcctcctgccccaagg

SEQ ID NO: 169 - DNA, 123 bp, Homo sapiens
tgaacgtcgtcacttgtaccaaacacacctaccgctgcctcaatgggctctgcttgagcaagggcaaccctgagtgtgacgggaaggaggactgtagcgacggctcagatgagaaggactgcg

SEQ ID NO: 170 - DNA, 187 bp, Homo sapiens
actgtgggctgcggtcattcacgagacaggctcgtgttgttgggggcacggatgcggatgagggcgagtggccctggcaggtaagcctgcatgctctgggccagggccacatctgcggtgcttccctcatctctcccaactggctggtctctgccgcacactgctacatcgatgacagaggattcag

SEQ ID NO: 171 - DNA, 275 bp, Homo sapiens
gtactcagaccccacgcagtggacggccttcctgggcttgcacgaccagagccagcgcagcgcccctggggtgcaggagcgcaggctcaagcgcatcatctcccaccccttcttcaatgacttcaccttcgactatgacatcgcgctgctggagctggagaaaccggcagagtacagctccatggtgcggcccatctgcctgccggacgcctcccatgtcttccctgccggcaaggccatctgggtcacgggctggggacacacccagtatggag

SEQ ID NO: 172 - DNA, 137 bp, Homo sapiens
gcactggcgcgctgatcctgcaaaagggtgagatccgcgtcatcaaccagaccacctgcgagaacctcctgccgcagcagatcacgccgcgcatgatgtgcgtgggcttcctcagcggcggcgtggactcctgccag

SEQ ID NO: 173 - DNA, 701 bp, Homo sapiens
ggtgattccgggggacccctgtccagcgtggaggcggatgggcggatcttccaggccggtgtggtgagctggggagacggctgcgctcagaggaacaagccaggcgtgtacacaaggctccctctgtttcgggactggatcaaagagaacactggggtataggggccggggccacccaaatgtgtacacctgcggggccacccatcgtccaccccagtgtgcacgcctgcaggctggagactggaccgctgactgcaccagcgcccccagaacatacactgtgaactcaatctccagggctccaaatctgcctagaaaacctctcgcttcctcagcctccaaagtggagctgggaggtagaaggggaggacactggtggttctactgacccaactgggggcaaaggtttgaagacacagcctcccccgccagccccaagctgggccgaggcgcgtttgtgcatatctgcctcccctgtctctaaggagcagcgggaacggagcttcggggcctcctcagtgaaggtggtggggctgccggatctgggctgtggggcccttgggccacgctcttgaggaagcccaggctcggaggaccctggaaaacagacgggtctgagactgaaattgttttaccagctcccagggtggacttcagtgtgtgtatttgtgtaaatgagtaaaacattttatttcttttta

SEQ ID NO: 174 - DNA, 3305 bp, Homo sapiens
gtgagagcggagctgcagccggagaaagaggaagagggagagagagcgcgccagggcgagggcaccgccgccggtcgggcgcgctgggcctgcccggaatcccgccgcctgcgccccgcgccccgcgccctgcgggccatgggagccggccgccggcagggacgacgcctgtgagacccgcgagcggcctcggggaccatggggagcgatcgggcccgcaagggcggagggggcccgaaggacttcggcgcgggactcaagtacaactcccggcacgagaaagtgaatggcttggaggaaggcgtggagttcctgccagtcaacaacgtcaagaaggtggaaaagcatggcccggggcgctgggtggtgctggcagccgtgctgatcggcctcctcttggtcttgctggggatcggcttcctggtgtggcatttgcagtaccgggacgtgcgtgtccagaaggtcttcaatggctacatgaggatcacaaatgagaattttgtggatgcctacgagaactccaactccactgagtttgtaagcctggccagcaaggtgaaggacgcgctgaagctgctgtacagcggagtcccattcctgggcccctaccacaaggagtcggctgtgacggccttcagcgagggcagcgtcatcgcctactactggtctgagttcagcatcccgcagcacctggtggaggaggccgagcgcgtcatggccgaggagcgcgtagtcatgctgcccccgcgggcgcgctccctgaagtcctttgtggtcacctcagtggtggctttccccacggactccaaaacagtacagaggacccaggacaacagctgcagctttggcctgcacgcccgcggtgtggagctgatgcgcttcaccacgcccggcttccctgacagcccctaccccgctcatgcccgctgccagtgggccctgcggggggacgccgactcagtgctgagcctcaccttccgcagctttgaccttgcgtcctgcgacgagcgcggcagcgacctggtgacggtgtacaacaccctgagccccatggagccccacgccctggtgcagttgtgtggcacctaccctccctcctacaacctgaccttccactcctcccagaacgtcctgctcatcacactgataaccaacactgagcggcggcatcccggctttgaggccaccttcttccagctgcctaggatgagcagctgtggaggccgcttacgtaaagcccaggggacattcaacagcccctactacccaggccactacccacccaacattgactgcacatggaacattgaggtgcccaacaaccagcatgtgaaggtgcgcttcaaattcttctacctgctggagcccggcgtgcctgcgggcacctgccccaaggactacgtggagatcaacggggagaaatactgcggagagaggtcccagttcgtcgtcaccagcaacagcaacaagatcacagttcgcttccactcagatcagtcctacaccgacaccggcttcttagctgaatacctctcctacgactccagtgacccatgcccggggcagttcacgtgccgcacggggcggtgtatccggaaggagctgcgctgtgatggctgggccgactgcaccgaccacagcgatgagctcaactgcagttgcgacgccggccaccagttcacgtgcaagaacaagttctgcaagcccctcttctgggtctgcgacagtgtgaacgactgcggagacaacagcgacgagcaggggtgcagttgtccggcccagaccttcaggtgttccaatgggaagtgcctctcgaaaagccagcagtgcaatgggaaggacgactgtggggacgggtccgacgaggcctcctgccccaaggtgaacgtcgtcacttgtaccaaacacacctaccgctgcctcaatgggctctgcttgagcaagggcaaccctgagtgtgacgggaaggaggactgtagcgacggctcagatgagaaggactgcgactgtgggctgcggtcattcacgagacaggctcgtgttgttgggggcacggatgcggatgagggcgagtggccctggcaggtaagcctgcatgctctgggccagggccacatctgcggtgcttccctcatctctcccaactggctggtctctgccgcacactgctacatcgatgacagaggattcaggtactcagaccccacgcagtggacggccttcctgggcttgcacgaccagagccagcgcagcgcccctggggtgcaggagcgcaggctcaagcgcatcatctcccaccccttcttcaatgacttcaccttcgactatgacatcgcgctgctggagctggagaaaccggcagagtacagctccatggtgcggcccatctgcctgccggacgcctcccatgtcttccctgccggcaaggccatctgggtcacgggctggggacacacccagtatggaggcactggcgcgctgatcctgcaaaagggtgagatccgcgtcatcaaccagaccacctgcgagaacctcctgccgcagcagatcacgccgcgcatgatgtgcgtgggcttcctcagcggcggcgtggactcctgccagggtgattccgggggacccctgtccagcgtggaggcggatgggcggatcttccaggccggtgtggtgagctggggagacggctgcgctcagaggaacaagccaggcgtgtacacaaggctccctctgtttcgggactggatcaaagagaacactggggtataggggccggggccacccaaatgtgtacacctgcggggccacccatcgtccaccccagtgtgcacgcctgcaggctggagactggaccgctgactgcaccagcgcccccagaacatacactgtgaactcaatctccagggctccaaatctgcctagaaaacctctcgcttcctcagcctccaaagtggagctgggaggtagaaggggaggacactggtggttctactgacccaactgggggcaaaggtttgaagacacagcctcccccgccagccccaagctgggccgaggcgcgtttgtgcatatctgcctcccctgtctctaaggagcagcgggaacggagcttcggggcctcctcagtgaaggtggtggggctgccggatctgggctgtggggcccttgggccacgctcttgaggaagcccaggctcggaggaccctggaaaacagacgggtctgagactgaaattgttttaccagctcccagggtggacttcagtgtgtgtatttgtgtaaatgagtaaaacattttatttcttttta

SEQ ID NO: 175 - DNA, 1552 bp, Homo sapiens
gtgagagcggagctgcagccggagaaagaggaagagggagagagagcgcgccagggcgagggcaccgccgccggtcgggcgcgctgggcctgcccggaatcccgccgcctgcgccccgcgccccgcgccctgcgggccatgggagccggccgccggcagggacgacgcctgtgagacccgcgagcggcctcggggaccatggggagcgatcgggcccgcaagggcggagggggcccgaaggacttcggcgcgggactcaagtacaactcccggcacgagaaagtgaatggcttggaggaaggcgtggagttcctgccagtcaacaacgtcaagaaggtggaaaagcatggcccggggcgctgggtggtgctggcagccgtgctgatcggcctcctcttggtcttgctggggatcggcttcctggtgtggcatttgcagtaccgggacgtgcgtgtccagaaggtcttcaatggctacatgaggatcacaaatgagaattttgtggatgcctacgagaactccaactccactgagtttgtaagcctggccagcaaggtgaaggacgcgctgaagctgctgtacagcggagtcccattcctgggcccctaccacaaggagtcggctgtgacggccttcagcgagggcagcgtcatcgcctactactggtctgagttcagcatcccgcagcacctggtggaggaggccgagcgcgtcatggccgaggagcgcgtagtcatgctgcccccgcgggcgcgctccctgaagtcctttgtggtcacctcagtggtggctttccccacggactccaaaacagtacagaggacccaggacaacagctgcagctttggcctgcacgcccgcggtgtggagctgatgcgcttcaccacgcccggcttccctgacagcccctaccccgctcatgcccgctgccagtgggccctgcggggggacgccgactcagtgctgagcctcaccttccgcagctttgaccttgcgtcctgcgacgagcgcggcagcgacctggtgacggtgtacaacaccctgagccccatggagccccacgccctggtgcagttgtgtggcacctaccctccctcctacaacctgaccttccactcctcccagaacgtcctgctcatcacactgataaccaacactgagcggcggcatcccggctttgaggccaccttcttccagctgcctaggatgagcagctgtggaggccgcttacgtaaagcccaggggacattcaacagcccctactacccaggccactacccacccaacattgactgcacatggaacattgaggtgcccaacaaccagcatgtgaaggtgcgcttcaaattcttctacctgctggagcccggcgtgcctgcgggcacctgccccaaggactacgtggagatcaacggggagaaatactgcggagagaggtcccagttcgtcgtcaccagcaacagcaacaagatcacagttcgcttccactcagatcagtcctacaccgacaccggcttcttagctgaatacctctcctacgactccagtgacc

SEQ ID NO: 176 - DNA, 145 bp, Homo sapiens
accctgtgaaggcgaagcgcgggagaccaaagcctgcaagaaagacgcctgccccactacatctacatccaccactgggacaagccatcttgtaaaatgtgcggagaaggagaaaactttctgtgtgaatggaggggagtgcttc

SEQ ID NO: 177 - DNA, 150 bp, Homo sapiens
agccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacag

SEQ ID NO: 178 - DNA, 96 bp, Homo sapiens
gatccctgctgggcaccaacagctccaccatggggctggcctggggactaggcgtcctgttcctgatgcatgtgtgtggcaccaaccgcattccag

SEQ ID NO: 179 - DNA, 560 bp, Homo sapiens
agtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccag

SEQ ID NO: 180 - DNA, 76 bp, Homo sapiens
ggggtgctgcagaatgtgaggtttgtctttggaaccacaccagaagacatcctcaggaacaaaggctgctccagct

SEQ ID NO: 181 - DNA, 200 bp, Homo sapiens
ctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtg

SEQ ID NO: 182 - DNA, 123 bp, Homo sapiens
actgaagagaacaaagagttggccaatgagctgaggcggcctcccctatgctatcacaacggagttcagtacagaaataacgaggaatggactgttgatagctgcactgagtgtcactgtcag

SEQ ID NO: 183 - DNA, 94 bp, Homo sapiens
aactcagttaccatctgcaaaaaggtgtcctgccccatcatgccctgctccaatgccacagttcctgatggagaatgctgtcctcgctgttggc

SEQ ID NO: 184 - DNA, 174 bp, Homo sapiens
ccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagat

SEQ ID NO: 185 - DNA, 177 bp, Homo sapiens
ttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgcccca

SEQ ID NO: 186 - DNA, 174 bp, Homo sapiens
tcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattg

SEQ ID NO: 187 - DNA, 128 bp, Homo sapiens
atggatgcctgtccaatccctgctttgccggcgtgaagtgtactagctaccctgatggcagctggaaatgtggtgcttgtccccctggttacagtggaaatggcatccagtgcacagatgttgatgag

SEQ ID NO: 188 - DNA, 153 bp, Homo sapiens
tgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacag

SEQ ID NO: 189 - DNA, 219 bp, Homo sapiens
gtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaag

SEQ ID NO: 190 - DNA, 108 bp, Homo sapiens
gataattgccccaaccttcccaactcagggcaggaagactatgacaaggatggaattggtgatgcctgtgatgatgacgatgacaatgataaaattccagatgacagg

SEQ ID NO: 191 - DNA, 160 bp, Homo sapiens
gacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacg

SEQ ID NO: 192 - DNA, 119 bp, Homo sapiens
gtatcctcaatgaacgggacaactgccagtacgtctacaatgtggaccagagagacactgatatggatggggttggagatcagtgtgacaattgccccttggaacacaatccggatcag

SEQ ID NO: 193 - DNA, 235 bp, Homo sapiens
ctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacg

SEQ ID NO: 194 - DNA, 228 bp, Homo sapiens
gcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtag

SEQ ID NO: 195 - DNA, 272 bp, Homo sapiens
gttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccag

SEQ ID NO: 196 - DNA, 98 bp, Homo sapiens
gtgcgcaccctgtggcatgaccctcgtcacataggctggaaagatttcaccgcctacagatggcgtctcagccacaggccaaagacgggtttcattag

SEQ ID NO: 197 - DNA, 140 bp, Homo sapiens
agtggtgatgtatgaagggaagaaaatcatggctgactcaggacccatctatgataaaacctatgctggtggtagactagggttgtttgtcttctctcaagaaatggtgttcttctctgacctgaaatacgaatgtagag

SEQ ID NO: 198 - DNA, 4105 bp, Homo sapiens
atccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcacctgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttcctgaattatcatggagttttctaattctctcttttggaatgtagattttttttaaatgctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattctttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagatatctcagattgtgtagatatgctatttaaataatttatcaggaaatactgcctgtagagttagtatttctatttttatataatgtttgcacactgaattgaagaattgttggttttttcttttttttgttttgtttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatatttatacttactgtatccatgcttatttgttctctactggctttatgtcatgaagtatatgcgtaaataccattcataaatcaatatagcatatacaaaaataaattacagtaagtcatagcaacattcacagtttgtatgtgattgagaaagactgagttgctcaggcctaggcttagaatttgctgcgtttgtggaataaaagaacaaaatgatacattagcctgccatatcaaaaacatataaaagagaaattatccctaagtcaagggcccccataagaataaaatttcttattaaggtcattagatgtcattgaatccttttcaaagtgcagtatgaaaacaaagggaaaaacactgaagcacacgcaactctcacagcgacattttctgacccacgaatgatgccttgggtgggcaacacgattgcatgttgtggagacacttcggaagtaaatgtggatgagggaggagctgtccttgcaatgttgagccaagcattacagatacctcctcttgaagaaggaataataagtttaatcaaaaaagaagactaaaaaatgtaaaatttggaaggaatccataaatgcgtgtgtgtctaaatacaaattatcatgtgaagaaaaggcccaagtgtaccaataagcagaccttgatttttggatgggctaattatgaatgtggaatactgaccagttaatttccagttttaatgaaaacagatcaaagaagaaattttatgagtaggttaaaggtctggctttgaggtctattaaacactagaaaggactggctgggtgagataaaatcttccttgttgattttcactctcattctataaatactcatctttctgagtagccatgatcacatacaaatgtaaattgccaaatcattttatagtaccaaggtgaagaagcaggaactagaaagtgttgataatagctgtggagttaggaaaactgatgtgaaggaaataattctttgaaatggcaaagaattaaataccatcattcattatcagaagagttcaacgtttgaagtgctgggagataattctaattcattcttggatagtgaagcaaaactgattgaaaataccaagataagacagaaaaagtgactggaaagaggagcttttcttccaggcatgttccagtttcaccctaagactgaccttcaaataatcaggttgtactgaaataaaggacttgttaaaaattaaaattatgtcatcgagatgatagcttttttcctcctccaacagtttattgtcatgtgttgtgggagagctcgagtgaagagcaataaactccaggtcttataagaatgtacatacaataaaggtggtgccagcagtttttttttttctaaagagtcacatgtagaaaagcctccagtattaagctcctgaattcattccttaaataaattggctctctctctcttctataatttctttttctttttatttttgagatgaagtcttgctctgtcgcccaggctggagtgcagtgacacaatctcggctcactgcaacctctgcctccccggttcaagcaattctccctcctgcctcagcctcccaagtagctgggactacaagcgcccgccaccaagcctggctaattctgtatttttagtaaagacggggtttcaccttgttccggacaaacactaagccctaaagggaaatccaaaataaaaacatctatttttaataacactttctatctaaatcagggtgactttttaaaaaaaatccggaagctttttgttgaattacgttacagacttagttaccagtccttgttagagttaccttcagttgacatgctgtgaatggtcccacctcttttatggcagaattcattacttaaaataactctattttcttcccccttacctaaataacagaaaggctcactatgtcccaaatatcattggcagaagcaaactataaagtcataagccctttgcagtgcaagtctagaaataatttt

SEQ ID NO: 199 - DNA, 7789 bp, Homo sapiens
agccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacaggatccctgctgggcaccaacagctccaccatggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattgatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcacctgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttcctgaattatcatggagttttctaattctctcttttggaatgtagattttttttaaatgctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattctttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagatatctcagattgtgtagatatgctatttaaataatttatcaggaaatactgcctgtagagttagtatttctatttttatataatgtttgcacactgaattgaagaattgttggttttttcttttttttgttttgtttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatatttatacttactgtatccatgcttatttgttctctactggctttatgtcatgaagtatatgcgtaaataccattcataaatcaatatagcatatacaaaaataaattacagtaagtcatagcaacattcacagtttgtatgtgattgagaaagactgagttgctcaggcctaggcttagaatttgctgcgtttgtggaataaaagaacaaaatgatacattagcctgccatatcaaaaacatataaaagagaaattatccctaagtcaagggcccccataagaataaaatttcttattaaggtcattagatgtcattgaatccttttcaaagtgcagtatgaaaacaaagggaaaaacactgaagcacacgcaactctcacagcgacattttctgacccacgaatgatgccttgggtgggcaacacgattgcatgttgtggagacacttcggaagtaaatgtggatgagggaggagctgtccttgcaatgttgagccaagcattacagatacctcctcttgaagaaggaataataagtttaatcaaaaaagaagactaaaaaatgtaaaatttggaaggaatccataaatgcgtgtgtgtctaaatacaaattatcatgtgaagaaaaggcccaagtgtaccaataagcagaccttgatttttggatgggctaattatgaatgtggaatactgaccagttaatttccagttttaatgaaaacagatcaaagaagaaattttatgagtaggttaaaggtctggctttgaggtctattaaacactagaaaggactggctgggtgagataaaatcttccttgttgattttcactctcattctataaatactcatctttctgagtagccatgatcacatacaaatgtaaattgccaaatcattttatagtaccaaggtgaagaagcaggaactagaaagtgttgataatagctgtggagttaggaaaactgatgtgaaggaaataattctttgaaatggcaaagaattaaataccatcattcattatcagaagagttcaacgtttgaagtgctgggagataattctaattcattcttggatagtgaagcaaaactgattgaaaataccaagataagacagaaaaagtgactggaaagaggagcttttcttccaggcatgttccagtttcaccctaagactgaccttcaaataatcaggttgtactgaaataaaggacttgttaaaaattaaaattatgtcatcgagatgatagcttttttcctcctccaacagtttattgtcatgtgttgtgggagagctcgagtgaagagcaataaactccaggtcttataagaatgtacatacaataaaggtggtgccagcagtttttttttttctaaagagtcacatgtagaaaagcctccagtattaagctcctgaattcattccttaaataaattggctctctctctcttctataatttctttttctttttatttttgagatgaagtcttgctctgtcgcccaggctggagtgcagtgacacaatctcggctcactgcaacctctgcctccccggttcaagcaattctccctcctgcctcagcctcccaagtagctgggactacaagcgcccgccaccaagcctggctaattctgtatttttagtaaagacggggtttcaccttgttccggacaaacactaagccctaaagggaaatccaaaataaaaacatctatttttaataacactttctatctaaatcagggtgactttttaaaaaaaatccggaagctttttgttgaattacgttacagacttagttaccagtccttgttagagttaccttcagttgacatgctgtgaatggtcccacctcttttatggcagaattcattacttaaaataactctattttcttcccccttacctaaataacagaaaggctcactatgtcccaaatatcattggcagaagcaaactataaagtcataagccctttgcagtgcaagtctagaaataatttt

SEQ ID NO: 200 - DNA, 1650 bp, Homo sapiens
agccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacaggatccctgctgggcaccaacagctccaccatggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgcccca

SEQ ID NO: 201 - DNA, 207 bp, Homo sapiens
gtgtgcggctcagatggggtcacctacagcaccgagtgtgagctgaagaaggccaggtgtgagtcacagcgagggctctacgtagcggcccagggagcctgccgagctacatctacatccaccactgggacaagccatcttgtaaaatgtgcggagaaggagaaaactttctgtgtgaatggaggggagtgcttcatggtgaaagac

SEQ ID NO: 202 - DNA, 254 bp, Homo sapiens
agtcccgtccccggcgcggcccgcgcgctcctccgccgcctctcgcctgcgccatggccggccggtcccacccgggcccgctgcggccgctgctgccgctccttgtggtggccgcgtgcgtcctgcccggagccggcgggacatgcccggagcgcgcgctggagcggcgcgaggaggaggcgaacgtggtgctcaccgggacggtggaggagatcctcaacgtggacccggtgcagcacacgtactcctgcaag

SEQ ID NO: 203 - DNA, 262 bp, Homo sapiens
gttcgggtctggcggtacttgaagggcaaagacctggtggcccgggagagcctgctggacggcggcaacaaggtggtgatcagcggctttggagaccccctcatctgtgacaaccaggtgtccactggggacaccaggatcttctttgtgaaccctgcacccccatacctgtggccagcccacaagaacgagctgatgctcaactccagcctcatgcggatcaccctgcggaacctggaggaggtggagttctgtgtggaag

SEQ ID NO: 204 - DNA, 48 bp, Homo sapiens
ataaacccgggacccacttcactccagtgcctccgacgcctcctgatg

SEQ ID NO: 205 - DNA, 216 bp, Homo sapiens
cgtgccggggaatgctgtgcggcttcggcgccgtgtgcgagcccaacgcggaggggccgggccgggcgtcctgcgtctgcaagaagagcccgtgccccagcgtggtggcgcctgtgtgtgggtcggacgcctccacctacagcaacgaatgcgagctgcagcgggcgcagtgcagccagcagcgccgcatccgcctgctcagccgcgggccgtgcg

SEQ ID NO: 206 - DNA, 225 bp, Homo sapiens
gctcgcgggacccctgctccaacgtgacctgcagcttcggcagcacctgtgcgcgctcggccgacgggctgacggcctcgtgcctgtgccccgcgacctgccgtggcgcccccgaggggaccgtctgcggcagcgacggcgccgactaccccggcgagtgccagctcctgcgccgcgcctgcgcccgccaggagaatgtcttcaagaagttcgacggcccttgtg

SEQ ID NO: 207 - DNA, 225 bp, Homo sapiens
acccctgtcagggcgccctccctgacccgagccgcagctgccgtgtgaacccgcgcacgcggcgccctgagatgctcctacggcccgagagctgccctgcccggcaggcgccagtgtgtggggacgacggagtcacctacgaaaacgactgtgtcatgggccgatcgggggccgcccggggtctcctcctgcagaaagtgcgctccggccagtgccagggtcgag

SEQ ID NO: 208 - DNA, 207 bp, Homo sapiens
accagtgcccggagccctgccggttcaatgccgtgtgcctgtcccgccgtggccgtccccgctgctcctgcgaccgcgtcacctgtgacggggcctacaggcccgtgtgtgcccaggacgggcgcacgtatgacagtgattgctggcggcagcaggctgagtgccggcagcagcgtgccatccccagcaagcaccagggcccgtgtg

SEQ ID NO: 209 - DNA, 219 bp, Homo sapiens
accaggccccgtccccatgcctcggggtgcagtgtgcatttggggcgacgtgtgctgtgaagaacgggcaggcagcgtgtgaatgcctgcaggcgtgctcgagcctctacgatcctgtgtgcggcagcgacggcgtcacatacggcagcgcgtgcgagctggaggccacggcctgtaccctcgggcgggagatccaggtggcgcgcaaaggaccctgtg

SEQ ID NO: 210 - DNA, 195 bp, Homo sapiens
accgctgcgggcagtgccgctttggagccctgtgcgaggccgagaccgggcgctgcgtgtgcccctctgaatgcgtggctttggcccagcccgtgtgtggctccgacgggcacacgtaccccagcgagtgcatgctgcacgtgcacgcctgcacacaccagatcagcctgcacgtggcctcagctggaccctgtg

SEQ ID NO: 211 - DNA, 201 bp, Homo sapiens
agacctgtggagatgccgtgtgtgcttttggggctgtgtgctccgcagggcagtgtgtgtgtccccggtgtgagcaccccccgcccggccccgtgtgtggcagcgacggtgtcacctacggcagtgcctgcgagctacgggaagccgcctgcctccagcagacacagatcgaggaggcccgggcagggccgtgcgagcagg

SEQ ID NO: 212 - DNA, 149 bp, Homo sapiens
ccgagtgcggttccggaggctctggctctggggaggacggtgactgtgagcaggagctgtgccggcagcgcggtggcatctgggacgaggactcggaggacgggccgtgtgtctgtgacttcagctgccagagtgtcccaggcagcccg

SEQ ID NO: 213 - DNA, 106 bp, Homo sapiens
gtgtgcggctcagatggggtcacctacagcaccgagtgtgagctgaagaaggccaggtgtgagtcacagcgagggctctacgtagcggcccagggagcctgccgag

SEQ ID NO: 214 - DNA, 117 bp, Homo sapiens
gccccaccttcgccccgctgccgcctgtggcccccttacactgtgcccagacgccctacggctgctgccaggacaatatcaccgcagcccggggcgtgggcctggctggctgcccca

SEQ ID NO: 215 - DNA, 7395 bp, Homo sapiens
agtcccgtccccggcgcggcccgcgcgctcctccgccgcctctcgcctgcgccatggccggccggtcccacccgggcccgctgcggccgctgctgccgctccttgtggtggccgcgtgcgtcctgcccggagccggcgggacatgcccggagcgcgcgctggagcggcgcgaggaggaggcgaacgtggtgctcaccgggacggtggaggagatcctcaacgtggacccggtgcagcacacgtactcctgcaaggttcgggtctggcggtacttgaagggcaaagacctggtggcccgggagagcctgctggacggcggcaacaaggtggtgatcagcggctttggagaccccctcatctgtgacaaccaggtgtccactggggacaccaggatcttctttgtgaaccctgcacccccatacctgtggccagcccacaagaacgagctgatgctcaactccagcctcatgcggatcaccctgcggaacctggaggaggtggagttctgtgtggaagataaacccgggacccacttcactccagtgcctccgacgcctcctgatgcgtgccggggaatgctgtgcggcttcggcgccgtgtgcgagcccaacgcggaggggccgggccgggcgtcctgcgtctgcaagaagagcccgtgccccagcgtggtggcgcctgtgtgtgggtcggacgcctccacctacagcaacgaatgcgagctgcagcgggcgcagtgcagccagcagcgccgcatccgcctgctcagccgcgggccgtgcggctcgcgggacccctgctccaacgtgacctgcagcttcggcagcacctgtgcgcgctcggccgacgggctgacggcctcgtgcctgtgccccgcgacctgccgtggcgcccccgaggggaccgtctgcggcagcgacggcgccgactaccccggcgagtgccagctcctgcgccgcgcctgcgcccgccaggagaatgtcttcaagaagttcgacggcccttgtgacccctgtcagggcgccctccctgacccgagccgcagctgccgtgtgaacccgcgcacgcggcgccctgagatgctcctacggcccgagagctgccctgcccggcaggcgccagtgtgtggggacgacggagtcacctacgaaaacgactgtgtcatgggccgatcgggggccgcccggggtctcctcctgcagaaagtgcgctccggccagtgccagggtcgagaccagtgcccggagccctgccggttcaatgccgtgtgcctgtcccgccgtggccgtccccgctgctcctgcgaccgcgtcacctgtgacggggcctacaggcccgtgtgtgcccaggacgggcgcacgtatgacagtgattgctggcggcagcaggctgagtgccggcagcagcgtgccatccccagcaagcaccagggcccgtgtgaccaggccccgtccccatgcctcggggtgcagtgtgcatttggggcgacgtgtgctgtgaagaacgggcaggcagcgtgtgaatgcctgcaggcgtgctcgagcctctacgatcctgtgtgcggcagcgacggcgtcacatacggcagcgcgtgcgagctggaggccacggcctgtaccctcgggcgggagatccaggtggcgcgcaaaggaccctgtgaccgctgcgggcagtgccgctttggagccctgtgcgaggccgagaccgggcgctgcgtgtgcccctctgaatgcgtggctttggcccagcccgtgtgtggctccgacgggcacacgtaccccagcgagtgcatgctgcacgtgcacgcctgcacacaccagatcagcctgcacgtggcctcagctggaccctgtgagacctgtggagatgccgtgtgtgcttttggggctgtgtgctccgcagggcagtgtgtgtgtccccggtgtgagcaccccccgcccggccccgtgtgtggcagcgacggtgtcacctacggcagtgcctgcgagctacgggaagccgcctgcctccagcagacacagatcgaggaggcccgggcagggccgtgcgagcaggccgagtgcggttccggaggctctggctctggggaggacggtgactgtgagcaggagctgtgccggcagcgcggtggcatctgggacgaggactcggaggacgggccgtgtgtctgtgacttcagctgccagagtgtcccaggcagcccggtgtgcggctcagatggggtcacctacagcaccgagtgtgagctgaagaaggccaggtgtgagtcacagcgagggctctacgtagcggcccagggagcctgccgaggccccaccttcgccccgctgccgcctgtggcccccttacactgtgcccagacgccctacggctgctgccaggacaatatcaccgcagcccggggcgtgggcctggctggctgccccagtgcctgccagtgcaacccccatggctcttacggcggcacctgtgacccagccacaggccagtgctcctgccgcccaggtgtggggggcctcaggtgtgaccgctgtgagcctggcttctggaactttcgaggcatcgtcaccgatggccggagtggctgtacaccctgcagctgtgatccccaaggcgccgtgcgggatgactgtgagcagatgacggggctgtgctcgtgtaagcccggggtggctggacccaagtgtgggcagtgtccagacggccgtgccctgggccccgcgggctgtgaagctgacgcttctgcgcctgcgacctgtgcggagatgcgctgtgagttcggtgcgcggtgcgtggaggagtctggctcagcccactgtgtctgcccgatgctcacctgtccagaggccaacgctaccaaggtctgtgggtcagatggagtcacatacggcaacgagtgtcagctgaagaccatcgcctgccgccagggcctgcaaatctctatccagagcctgggcccgtgccaggaggctgttgctcccagcactcacccgacatctgcctccgtgactgtgaccaccccagggctcctcctgagccaggcactgccggccccccccggcgccctccccctggctcccagcagtaccgcacacagccagaccacccctccgccctcatcacgacctcggaccactgccagcgtccccaggaccaccgtgtggcccgtgctgacggtgccccccacggcaccctcccctgcacccagcctggtggcgtccgcctttggtgaatctggcagcactgatggaagcagcgatgaggaactgagcggggaccaggaggccagtgggggtggctctggggggctcgagcccttggagggcagcagcgtggccacccctgggccacctgtcgagagggcttcctgctacaactccgcgttgggctgctgctctgatgggaagacgccctcgctggacgcagagggctccaactgccccgccaccaaggtgttccagggcgtcctggagctggagggcgtcgagggccaggagctgttctacacgcccgagatggctgaccccaagtcagaactgttcggggagacagccaggagcattgagagcaccctggacgacctcttccggaattcagacgtcaagaaggattttcggagtgtccgcttgcgggacctggggcccggcaaatccgtccgcgccattgtggatgtgcactttgaccccaccacagccttcagggcacccgacgtggcccgggccctgctccggcagatccaggtgtccaggcgccggtccttgggggtgaggcggccgctgcaggagcacgtgcgatttatggactttgactggtttcctgcgtttatcacgggggccacgtcaggagccattgctgcgggagccacggccagagccaccactgcatcgcgcctgccgtcctctgctgtgacccctcgggccccgcaccccagtcacacaagccagcccgttgccaagaccacggcagcccccaccacacgtcggccccccaccactgcccccagccgtgtgcccggacgtcggcccccggccccccagcagcctccaaagccctgtgactcacagccctgcttccacggggggacctgccaggactgggcattgggcgggggcttcacctgcagctgcccggcaggcaggggaggcgccgtctgtgagaaggtgcttggcgcccctgtgccggccttcgagggccgctccttcctggccttccccactctccgcgcctaccacacgctgcgcctggcactggaattccgggcgctggagcctcaggggctgctgctgtacaatggcaacgcccggggcaaggacttcctggcattggcgctgctagatggccgcgtgcagctcaggtttgacacaggttcggggccggcggtgctgaccagtgccgtgccggtagagccgggccagtggcaccgcctggagctgtcccggcactggcgccggggcaccctctcggtggatggtgagacccctgttctgggcgagagtcccagtggcaccgacggcctcaacctggacacagacctctttgtgggcggcgtacccgaggaccaggctgccgtggcgctggagcggaccttcgtgggcgccggcctgagggggtgcatccgtttgctggacgtcaacaaccagcgcctggagcttggcattgggccgggggctgccacccgaggctctggcgtgggcgagtgcggggaccacccctgcctgcccaacccctgccatggcggggccccatgccagaacctggaggctggaaggttccattgccagtgcccgcccggccgcgtcggaccaacctgtgccgatgagaagagcccctgccagcccaacccctgccatggggcggcgccctgccgtgtgctgcccgagggtggtgctcagtgcgagtgccccctggggcgtgagggcaccttctgccagacagcctcggggcaggacggctctgggcccttcctggctgacttcaacggcttctcccacctggagctgagaggcctgcacacctttgcacgggacctgggggagaagatggcgctggaggtcgtgttcctggcacgaggccccagcggcctcctgctctacaacgggcagaagacggacggcaagggggacttcgtgtcgctggcactgcgggaccgccgcctggagttccgctacgacctgggcaagggggcagcggtcatcaggagcagggagccagtcaccctgggagcctggaccagggtctcactggagcgaaacggccgcaagggtgccctgcgtgtgggcgacggcccccgtgtgttgggggagtccccgaaatcccgcaaggttccgcacaccgtcctcaacctgaaggagccgctctacgtagggggcgctcccgacttcagcaagctggcccgtgctgctgccgtgtcctctggcttcgacggtgccatccagctggtctccctcggaggccgccagctgctgaccccggagcacgtgctgcggcaggtggacgtcacgtcctttgcaggtcacccctgcacccgggcctcaggccacccctgcctcaatggggcctcctgcgtcccgagggaggctgcctatgtgtgcctgtgtcccgggggattctcaggaccgcactgcgagaaggggctggtggagaagtcagcgggggacgtggataccttggcctttgacgggcggacctttgtcgagtacctcaacgctgtgaccgagagcgaactggccaatgagatccccgtccccgaaactctggattccggggcccttcacagcgagaaggcactgcagagcaaccactttgaactgagcctgcgcactgaggccacgcaggggctggtgctctggagtggcaaggccacggagcgggcagactatgtggcactggccattgtggacgggcacctgcaactgagctacaacctgggctcccagcccgtggtgctgcgttccaccgtgcccgtcaacaccaaccgctggttgcgggtcgtggcacatagggagcagagggaaggttccctgcaggtgggcaatgaggcccctgtgaccggctcctccccgctgggcgccacgcagctggacactgatggagccctgtggcttgggggcctgccggagctgcccgtgggcccagcactgcccaaggcctacggcacaggctttgtgggctgcttgcgggacgtggtggtgggccggcacccgctgcacctgctggaggacgccgtcaccaagccagagctgcggccctgccccaccccatgagctggcaccagagccccgcgcccgctgtaattattttctatttttgtaaacttgttgctttttgatatgattttcttgcctgagtgttggccggagggactgctggcccggcctcccttccgtccaggcagccgtgctgcagacagacctagtgccgagggatggacaggcgaggtggcagcgtggagggctcggcgtggatggcagcctcaggacacacacccctgcctcaaggtgctgagcccccgccttgcactgcgcctgccccacggtgtccccgccgggaagcagccccggctcctgaatcaccctcgctccgtcaggcgggactcgtgtcccagagaggaaggggctgctgaggtctgatggggcccttcctccgggtgaccccacagggcctttccaagcccccatttgagctgctccttcctgtgtgtgctctgggccctgcctcggcctcctgcgccaatactgtgacttccaaacaatgttactgctgggcacagctctgcgttgctcccgtgctgcctgcgccagccccaggctgctgaggagcagaggccagaccagggccgatctgggtgtcctgaccctcagctggccctgcccagccaccctggacgtgaccgtatccctctgccacaccccaggccctgcgaggggctatcgagaggagctcactgtgggatggggttgacctctgccgcctgcctgggtatctgggcctggccatggctgtgttcttcatgtgttgattttatttgacccctggagtggtgggtctcatctttcccatctcgcctgagagcggctgagggctgcctcactgcaaatcctccccacagcgtcagtgaaagtcgtccttgtctcagaatgaccaggggccagccagtgtctgaccaaggtcaaggggcaggtgcagaggtggcagggatggctccgaagccagaaatgccttaaactgcaacgtcccgtcccttccccacccccatcccatccccacccccagccccagcccagtcctcctaggagcaggacccgatgaagcgggcggcggtggggctgggtgccgtgttactaactctagtatgtttctgtgtcaatcgctgtgaaataaagtctgaaaactttaaaa

SEQ ID NO: 216 - DNA, 2307 bp, Homo sapiens
agtcccgtccccggcgcggcccgcgcgctcctccgccgcctctcgcctgcgccatggccggccggtcccacccgggcccgctgcggccgctgctgccgctccttgtggtggccgcgtgcgtcctgcccggagccggcgggacatgcccggagcgcgcgctggagcggcgcgaggaggaggcgaacgtggtgctcaccgggacggtggaggagatcctcaacgtggacccggtgcagcacacgtactcctgcaaggttcgggtctggcggtacttgaagggcaaagacctggtggcccgggagagcctgctggacggcggcaacaaggtggtgatcagcggctttggagaccccctcatctgtgacaaccaggtgtccactggggacaccaggatcttctttgtgaaccctgcacccccatacctgtggccagcccacaagaacgagctgatgctcaactccagcctcatgcggatcaccctgcggaacctggaggaggtggagttctgtgtggaagataaacccgggacccacttcactccagtgcctccgacgcctcctgatgcgtgccggggaatgctgtgcggcttcggcgccgtgtgcgagcccaacgcggaggggccgggccgggcgtcctgcgtctgcaagaagagcccgtgccccagcgtggtggcgcctgtgtgtgggtcggacgcctccacctacagcaacgaatgcgagctgcagcgggcgcagtgcagccagcagcgccgcatccgcctgctcagccgcgggccgtgcggctcgcgggacccctgctccaacgtgacctgcagcttcggcagcacctgtgcgcgctcggccgacgggctgacggcctcgtgcctgtgccccgcgacctgccgtggcgcccccgaggggaccgtctgcggcagcgacggcgccgactaccccggcgagtgccagctcctgcgccgcgcctgcgcccgccaggagaatgtcttcaagaagttcgacggcccttgtgacccctgtcagggcgccctccctgacccgagccgcagctgccgtgtgaacccgcgcacgcggcgccctgagatgctcctacggcccgagagctgccctgcccggcaggcgccagtgtgtggggacgacggagtcacctacgaaaacgactgtgtcatgggccgatcgggggccgcccggggtctcctcctgcagaaagtgcgctccggccagtgccagggtcgagaccagtgcccggagccctgccggttcaatgccgtgtgcctgtcccgccgtggccgtccccgctgctcctgcgaccgcgtcacctgtgacggggcctacaggcccgtgtgtgcccaggacgggcgcacgtatgacagtgattgctggcggcagcaggctgagtgccggcagcagcgtgccatccccagcaagcaccagggcccgtgtgaccaggccccgtccccatgcctcggggtgcagtgtgcatttggggcgacgtgtgctgtgaagaacgggcaggcagcgtgtgaatgcctgcaggcgtgctcgagcctctacgatcctgtgtgcggcagcgacggcgtcacatacggcagcgcgtgcgagctggaggccacggcctgtaccctcgggcgggagatccaggtggcgcgcaaaggaccctgtgaccgctgcgggcagtgccgctttggagccctgtgcgaggccgagaccgggcgctgcgtgtgcccctctgaatgcgtggctttggcccagcccgtgtgtggctccgacgggcacacgtaccccagcgagtgcatgctgcacgtgcacgcctgcacacaccagatcagcctgcacgtggcctcagctggaccctgtgagacctgtggagatgccgtgtgtgcttttggggctgtgtgctccgcagggcagtgtgtgtgtccccggtgtgagcaccccccgcccggccccgtgtgtggcagcgacggtgtcacctacggcagtgcctgcgagctacgggaagccgcctgcctccagcagacacagatcgaggaggcccgggcagggccgtgcgagcaggccgagtgcggttccggaggctctggctctggggaggacggtgactgtgagcaggagctgtgccggcagcgcggtggcatctgggacgaggactcggaggacgggccgtgtgtctgtgacttcagctgccagagtgtcccaggcagcccggtgtgcggctcagatggggtcacctacagcaccgagtgtgagctgaagaaggccaggtgtgagtcacagcgagggctctacgtagcggcccagggagcctgccgag

SEQ ID NO: 217 - DNA, 227 bp, Homo sapiens
taaaaggcttctccccagatgccagagacctgtctgctaaagaaaccaagatgctgatggctgctggagacaaagatggggacggcaaaattggggttgacgctacatctacatccaccactgggacaagccatcttgtaaaatgtgcggagaaggagaaaactttctgtgtgaatggaggggagtgcttcatggtgaaagacctttcaaacccctcgagatacttg

SEQ ID NO: 218 - DNA, 69 bp, Homo sapiens
acttcccgacaggacttcccaccagcccagcctttcagtgcaggctccagccctccacccccacccgag

SEQ ID NO: 219 - DNA, 68 bp, Homo sapiens
ttgcaggatgtcgatgacagacttgctgaacgctgaggacatcaagaaggcggtgggagcctttagcg

SEQ ID NO: 220 - DNA, 133 bp, Homo sapiens
ctaccgactccttcgaccacaaaaagttcttccaaatggtcggcctgaagaaaaagagtgcggatgatgtgaagaaggtgtttcacatgctggacaaggacaaaagtggcttcatcgaggaggatgagctggg

SEQ ID NO: 221 - DNA, 110 bp, Homo sapiens
attcatcctaaaaggcttctccccagatgccagagacctgtctgctaaagaaaccaagatgctgatggctgctggagacaaagatggggacggcaaaattggggttgacg

SEQ ID NO: 222 - DNA, 216 bp, Homo sapiens
aattctccactctggtggctgaaagctaagaagcactgactgcccctggtcttccacctctctgccctgaacacccaatctcggcccctctcgccaccctcctgcatttctgttcagttcgtttatgttattttttactcccccatcccctgtggccctctaatgacaccattcttctggaaaatgctggagaagcaataaaggttgtaccagtca

SEQ ID NO: 223 - DNA, 596 bp, Homo sapiens
acttcccgacaggacttcccaccagcccagcctttcagtgcaggctccagccctccacccccacccgagttgcaggatgtcgatgacagacttgctgaacgctgaggacatcaagaaggcggtgggagcctttagcgctaccgactccttcgaccacaaaaagttcttccaaatggtcggcctgaagaaaaagagtgcggatgatgtgaagaaggtgtttcacatgctggacaaggacaaaagtggcttcatcgaggaggatgagctgggattcatcctaaaaggcttctccccagatgccagagacctgtctgctaaagaaaccaagatgctgatggctgctggagacaaagatggggacggcaaaattggggttgacgaattctccactctggtggctgaaagctaagaagcactgactgcccctggtcttccacctctctgccctgaacacccaatctcggcccctctcgccaccctcctgcatttctgttcagttcgtttatgttattttttactcccccatcccctgtggccctctaatgacaccattcttctggaaaatgctggagaagcaataaaggttgtaccagtca

SEQ ID NO: 224 - DNA, 380 bp, Homo sapiens
acttcccgacaggacttcccaccagcccagcctttcagtgcaggctccagccctccacccccacccgagttgcaggatgtcgatgacagacttgctgaacgctgaggacatcaagaaggcggtgggagcctttagcgctaccgactccttcgaccacaaaaagttcttccaaatggtcggcctgaagaaaaagagtgcggatgatgtgaagaaggtgtttcacatgctggacaaggacaaaagtggcttcatcgaggaggatgagctgggattcatcctaaaaggcttctccccagatgccagagacctgtctgctaaagaaaccaagatgctgatggctgctggagacaaagatggggacggcaaaattggggttgacg

SEQ ID NO: 225 - DNA, 121 bp, Homo sapiens
agttggggtctcactgtgttgcccagactggtctcgaactcttggcctcaggtgatcctcttccctcagcttcccagaatgccgagatgatagctacatctacatccaccactgggacaag

SEQ ID NO: 226 - DNA, 273 bp, Homo sapiens
gcattgcggcttggttttctcacccagtgcatgtggcaggagcggtgagatcactgcctcacggcgatcctggactgacggtcacgactgcctaccctctaaccctgttctgagctgccccttgcccacacaccccaaacctgtgtgcaggatccgcctccatggagctacagcctcctgaagcctcgatcgccgtcgtgtcgattccgcgccagttgcctggctcacattcggaggctggtgtccagggtctcagcgcgggggacgactcag

SEQ ID NO: 227 - DNA, 93 bp, Homo sapiens
agttggggtctcactgtgttgcccagactggtctcgaactcttggcctcaggtgatcctcttccctcagcttcccagaatgccgagatgatag

SEQ ID NO: 228 - DNA, 93 bp, Homo sapiens
agacggggtctgactgtgttacccaggctggtcttcaactcttggcctcaagtgatcctcctgccttagcttccaagaatgctgaggttacag

SEQ ID NO: 229 - DNA, 429 bp, Homo sapiens
gcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgg

SEQ ID NO: 230 - DNA, 174 bp, Homo sapiens; identical to SEQ ID NO: 64

SEQ ID NO: 231 - DNA, 92 bp, Homo sapiens; identical to SEQ ID NO: 65

SEQ ID NO: 232 - DNA, 69 bp, Homo sapiens; identical to SEQ ID NO: 66

SEQ ID NO: 233 - DNA, 59 bp, Homo sapiens; identical to SEQ ID NO: 67

SEQ ID NO: 234 - DNA, 181 bp, Homo sapiens; identical to SEQ ID NO: 68

SEQ ID NO: 235 - DNA, 144 bp, Homo sapiens; identical to SEQ ID NO: 69

SEQ ID NO: 236 - DNA, 84 bp, Homo sapiens; identical to SEQ ID NO: 70

SEQ ID NO: 237 - DNA, 530 bp, Homo sapiens; identical to SEQ ID NO: 71

SEQ ID NO: 238 - DNA, 2221 bp, Homo sapiens
gcattgcggcttggttttctcacccagtgcatgtggcaggagcggtgagatcactgcctcacggcgatcctggactgacggtcacgactgcctaccctctaaccctgttctgagctgccccttgcccacacaccccaaacctgtgtgcaggatccgcctccatggagctacagcctcctgaagcctcgatcgccgtcgtgtcgattccgcgccagttgcctggctcacattcggaggctggtgtccagggtctcagcgcgggggacgactcagagttggggtctcactgtgttgcccagactggtctcgaactcttggcctcaggtgatcctcttccctcagcttcccagaatgccgagatgatagagacggggtctgactgtgttacccaggctggtcttcaactcttggcctcaagtgatcctcctgccttagcttccaagaatgctgaggttacaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctgacttcagcctgacatggacccactacccttctcctttccttcccaggccctttggcttctgatttttctcttttttaaaaacaaacaaacaaactgttgcagattatgagtgaacccccaaatagggtgttttctgccttcaaataaaagtcacccctgcatggtgaa

SEQ ID NO: 239 - DNA, 366 bp, Homo sapiens
gcattgcggcttggttttctcacccagtgcatgtggcaggagcggtgagatcactgcctcacggcgatcctggactgacggtcacgactgcctaccctctaaccctgttctgagctgccccttgcccacacaccccaaacctgtgtgcaggatccgcctccatggagctacagcctcctgaagcctcgatcgccgtcgtgtcgattccgcgccagttgcctggctcacattcggaggctggtgtccagggtctcagcgcgggggacgactcagagttggggtctcactgtgttgcccagactggtctcgaactcttggcctcaggtgatcctcttccctcagcttcccagaatgccgagatgatag

SEQ ID NO: 240 - DNA, 141 bp, Homo sapiens
ttgagcctgttgatgcccgccctgctgccgaccgaggactgaccactcgaccagctacatctacatccaccactgggacaagccatcttgtaaaatgtgcggagaaggagaaaactttctgtgtgaatggaggggagtgct

SEQ ID NO: 241 - DNA, 207 bp, Homo sapiens
gtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggag

SEQ ID NO: 242 - DNA, 130 bp, Homo sapiens
gtctaccctgaactgcagatcaccaatgtggtagaagccaaccaaccagtgaccatccagaactggtgcaagcggggccgcaagcagtgcaagacccatccccactttgtgattccctaccgctgcttag

SEQ ID NO: 243 - DNA, 113 bp, Homo sapiens
ttggtgagtttgtaagtgatgcccttctcgttcctgacaagtgcaaattcttacaccaggagaggatggatgtttgcgaaactcatcttcactggcacaccgtcgccaaagag

SEQ ID NO: 244 - DNA, 194 bp, Homo sapiens
acatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggag

SEQ ID NO: 245 - DNA, 203 bp, Homo sapiens
tgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgag

SEQ ID NO: 246 - DNA, 168 bp, Homo sapiens
aggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgcca

SEQ ID NO: 247 - DNA, 57 bp, Homo sapiens
tgtcccaaagtttactcaagactacccaggaacctcttgcccgagatcctgttaaac

SEQ ID NO: 248 - DNA, 134 bp, Homo sapiens
ttcctacaacagcagccagtacccctgatgccgttgacaagtatctcgagacacctggggatgagaatgaacatgcccatttccagaaagccaaagagaggcttgaggccaagcaccgagagagaatgtcccag

SEQ ID NO: 249 - DNA, 75 bp, Homo sapiens
gtcatgagagaatgggaagaggcagaacgtcaagcaaagaacttgcctaaagctgataagaaggcagttatccag

SEQ ID NO: 250 - DNA, 159 bp, Homo sapiens
catttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcgg

SEQ ID NO: 251 - DNA, 129 bp, Homo sapiens
cctcgtcacgtgttcaatatgctaaagaagtatgtccgcgcagaacagaaggacagacagcacaccctaaagcatttcgagcatgtgcgcatggtggatcccaagaaagccgctcagatccggtcccag

SEQ ID NO: 252 - DNA, 100 bp, Homo sapiens
gttatgacacacctccgtgtgatttatgagcgcatgaatcagtctctctccctgctctacaacgtgcctgcagtggccgaggagattcaggatgaagttg

SEQ ID NO: 253 - DNA, 222 bp, Homo sapiens
atgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaag

SEQ ID NO: 254 - DNA, 54 bp, Homo sapiens
ttgagcctgttgatgcccgccctgctgccgaccgaggactgaccactcgaccag

SEQ ID NO: 255 - DNA, 101 bp, Homo sapiens
gttctgggttgacaaatatcaagacggaggagatctctgaagtgaagatggatgcagaattccgacatgactcaggatatgaagttcatcatcaaaaattg

SEQ ID NO: 256 - DNA, 147 bp, Homo sapiens
gtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggag

SEQ ID NO: 257 - DNA, 1222 bp, Homo sapiens
gttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagca

SEQ ID NO: 258 - DNA, 3415 bp, Homo sapiens
gtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagca

SEQ ID NO: 259 - DNA, 1945 bp, Homo sapiens
gtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccag

SEQ ID NO: 260 - DNA, 182 bp, Homo sapiens
aagctggctgcccccttgatttggagcgagcaggcctgactccagaggttcagaagattattgctgatgttatccgaaaccctcccgtcaactcagctacatctacatccaccactgggacaagccatcttgtaaaatgtgcggagaaggagaaaactttctgtgtgaatggaggggagtgc

SEQ ID NO: 261 - DNA, 164 bp, Homo sapiens
gtgtactgtgtgcgccggggaggcgccggcttgtactcggcagcgcgggaataaagtttgctgatttggtgtctagcctggatgcctgggttgcaggccctgcttgtggtggcgctccacagtcatccggctgaagaagacctgttggactggatcttctcggg

SEQ ID NO: 262 - DNA, 172 bp, Homo sapiens
ttttctttcagatattgttttgtatttacccatgaagacattgttttttggactctgcaaataggacatttcaaagatgagtgaaaaaaaattggaaacaactgcacagcagcggaaatgtcctgaatggatgaatgtgcagaataaaagatgtgctgtagaagaaagaaag

SEQ ID NO: 263 - DNA, 113 bp, Homo sapiens
gcatgtgttcggaagagtgtttttgaagatgacctccccttcttagaattcactggatccattgtgtatagttacgatgctagtgattgctctttcctgtcagaagatattag

SEQ ID NO: 264 - DNA, 146 bp, Homo sapiens
catgagtctatcagatggggatgtggtgggatttgacatggagtggccaccattatacaatagagggaaacttggcaaagttgcactaattcagttgtgtgtttctgagagcaaatgttacttgttccacgtttcttccatgtcag

SEQ ID NO: 265 - DNA, 149 bp, Homo sapiens
tttttccccagggattaaaaatgttgcttgaaaataaagcagttaaaaaggcaggtgtaggaattgaaggagatcagtggaaacttctacgtgactttgatatcaaattgaagaattttgtggagttgacagatgttgccaataaaaag

SEQ ID NO: 266 - DNA, 150 bp, Homo sapiens
ctgaaatgcacagagacctggagccttaacagtctggttaaacacctcttaggtaaacagctcctgaaagacaagtctatccgctgtagcaattggagtaaatttcctctcactgaggaccagaaactgtatgcagccactgatgcttat

SEQ ID NO: 267 - DNA, 70 bp, Homo sapiens
gctggttttattatttaccgaaatttagagattttggatgatactgtgcaaaggtttgctataaataaag

SEQ ID NO: 268 - DNA, 115 bp, Homo sapiens
aggaagaaatcctacttagcgacatgaacaaacagttgacttcaatctctgaggaagtgatggatctggctaagcatcttcctcatgctttcagtaaattggaaaacccacggag

SEQ ID NO: 269 - DNA, 430 bp, Homo sapiens
ggtttctatcttactaaaggatatttcagaaaatctatattcactgaggaggatgataattgggtctactaacattgagactgaactgaggcccagcaataatttaaacttattatcctttgaagattcaactactgggggagtacaacagaaacaaattagagaacatgaagttttaattcacgttgaagatgaaacatgggacccaacacttgatcatttagctaaacatgatggagaagatgtacttggaaataaagtggaacgaaaagaagatggatttgaagatggagtagaagacaacaaattgaaagagaatatggaaagagcttgtttgatgtcgttagatattacagaacatgaactccaaattttggaacagcagtctcaggaagaatatcttagtgatattgcttataaatctactgag

SEQ ID NO: 270 - DNA, 81 bp, Homo sapiens
catttatctcccaatgataatgaaaacgatacgtcctatgtaattgagagtgatgaagatttagaaatggagatgcttaag

SEQ ID NO: 271 - DNA, 81 bp, Homo sapiens; identical to SEQ ID NO: 270

SEQ ID NO: 272 - DNA, 145 bp, Homo sapiens
tctttagaaaacctcaatagtggcacggtagaaccaactcattctaaatgcttaaaaatggaaagaaatctgggtcttcctactaaagaagaagaagaagatgatgaaaatgaagctaatgaaggggaagaagatgatgataagg

SEQ ID NO: 273 - DNA, 76 bp, Homo sapiens
actttttgtggccagcacccaatgaagagcaagttacttgcctcaagatgtactttggccattccagttttaaacc

SEQ ID NO: 274 - DNA, 68 bp, Homo sapiens
agttcagtggaaagtgattcattcagtattagaagaaagaagagataatgttgctgtcatggcaactg

SEQ ID NO: 275 - DNA, 109 bp, Homo sapiens
gatatggaaagagtttgtgcttccagtatccacctgtttatgtaggcaagattggccttgttatctctccccttatttctctgatggaagaccaagtgctacagcttaa

SEQ ID NO: 276 - DNA, 69 bp, Homo sapiens
aatgtccaacatcccagcttgcttccttggatcagcacagtcagaaaatgttctaacagatattaaatt

SEQ ID NO: 277 - DNA, 83 bp, Homo sapiens
aggtaaataccggattgtatacgtaactccagaatactgttcaggtaacatgggcctgctccagcaacttgaggctgatattg

SEQ ID NO: 278 - DNA, 107 bp, Homo sapiens
gtatcacgctcattgctgtggatgaggctcactgtatttctgagtgggggcatgattttagggattcattcaggaagttgggctccctaaagacagcactgccaatg

SEQ ID NO: 279 - DNA, 185 bp, Homo sapiens
gttccaatcgttgcacttactgctactgcaagttcttcaatccgggaagacattgtacgttgcttaaatctgagaaatcctcagatcacctgtactggttttgatcgaccaaacctgtatttagaagttaggcgaaaaacagggaatatccttcaggatctgcagccatttcttgtcaaaacaag

SEQ ID NO: 280 - DNA, 175 bp, Homo sapiens
ttcccactgggaatttgaaggtccaacaatcatctactgtccttctagaaaaatgacacaacaagttacaggtgaacttaggaaactgaatctatcctgtggaacataccatgcgggcatgagttttagcacaaggaaagacattcatcataggtttgtaagagatgaaattcag

SEQ ID NO: 281 - DNA, 182 bp, Homo sapiens
tgtgtcatagctaccatagcttttggaatgggcattaataaagctgacattcgccaagtcattcattacggtgctcctaaggacatggaatcatattatcaggagattggtagagctggtcgtgatggacttcaaagttcttgtcacgtcctctgggctcctgcagacattaacttaaatag

SEQ ID NO: 282 - DNA, 102 bp, Homo sapiens
gcaccttcttactgagatacgtaatgagaagtttcgattatacaaattaaagatgatggcaaagatggaaaaatatcttcattctagcagatgtaggagaca

SEQ ID NO: 283 - DNA, 93 bp, Homo sapiens
aatcatcttgtctcattttgaggacaaacaagtacaaaaagcctccttgggaattatgggaactgaaaaatgctgtgataattgcaggtccag

SEQ ID NO: 284 - DNA, 142 bp, Homo sapiens
attggatcattgctattccatggatgactcagaggatacatcctgggactttggtccacaagcatttaagcttttgtctgctgtggacatcttaggcgaaaaatttggaattgggcttccaattttatttctccgaggatct

SEQ ID NO: 285 - DNA, 171 bp, Homo sapiens
aattctcagcgtcttgccgatcaatatcgcaggcacagtttatttggcactggcaaggatcaaacagagagttggtggaaggctttttcccgtcagctgatcactgagggattcttggtagaagtttctcggtataacaaatttatgaagatttgcgcccttacgaaaaag

SEQ ID NO: 286 - DNA, 95 bp, Homo sapiens
ggtagaaattggcttcataaagctaatacagaatctcagagcctcatccttcaagctaatgaagaattgtgtccaaagaagttgcttctgcctag

SEQ ID NO: 287 - DNA, 76 bp, Homo sapiens
ttcgaaaactgtatcttcgggcaccaaagagcattgttataatcaagtaccagttgaattaagtacagagaagaag

SEQ ID NO: 288 - DNA, 74 bp, Homo sapiens
tctaacttggagaagttatattcttataaaccatgtgataagatttcttctgggagtaacatttctaaaaaaag

SEQ ID NO: 289 - DNA, 76 bp, Homo sapiens
tatcatggtacagtcaccagaaaaagcttacagttcctcacagcctgttatttcggcacaagagcaggagactcag

SEQ ID NO: 290 - DNA, 113 bp, Homo sapiens
attgtgttatatggcaaattggtagaagctaggcagaaacatgccaataaaatggatgttcccccagctattctggcaacaaacaagatactggtggatatggccaaaatgag

SEQ ID NO: 291 - DNA, 115 bp, Homo sapiens
accaactacggttgaaaacgtaaaaaggattgatggtgtttctgaaggcaaagctgccatgttggcccctctgttggaagtcatcaaacatttctgccaaacaaatagtgttcag

SEQ ID NO: 292 - DNA, 132 bp, Homo sapiens
acagacctcttttcaagtacaaaacctcaagaagaacagaagacgagtctggtagcaaaaaataaaatatgcacactttcacagtctatggccatcacatactctttattccaagaaaagaagatgcctttg

SEQ ID NO: 293 - DNA, 163 bp, Homo sapiens
aagagcatagctgagagcaggattctgcctctcatgacaattggcatgcacttatcccaagcggtgaaagctggctgcccccttgatttggagcgagcaggcctgactccagaggttcagaagattattgctgatgttatccgaaaccctcccgtcaactcag

SEQ ID NO: 294 - DNA, 209 bp, Homo sapiens
atatgagtaaaattagcctaatcagaatgttagttcctgaaaacattgacacgtaccttatccacatggcaattgagatccttaaacatggtcctgacagcggacttcaaccttcatgtgatgtcaacaaaaggagatgttttcccggttctgaagagatctgttcaagttctaagagaagcaaggaagaagtaggcatcaatactgag

SEQ ID NO: 295 - DNA, 3144 bp, Homo sapiens
acttcatctgcagagagaaagagacgattacctgtgtggtttgccaaaggaagtgataccagcaagaaattaatggacaaaacgaaaaggggaggtctttttagttaagctggcaattaccagaacaattatgtttcttgctgtattataagaggatagctatattttatttctgaagagtaaggagtagtattttggcttaaaaatcattctaattacaaagttcactgtttattgaagaactggcatcttaaatcagccttccgcaattcatgtagtttctgggtcttctgggagcctacgtgagtacatcacctaacagaatattaaattagacttcctgtaagattgctttaagaaactgttactgtcctgttttctaatctctttattaaaacagtgtatttggaaaatgttatgtgctctgatttgatatagataacagattagtagttacatggtaattatgtgatataaaatattcatatattatcaaaattctgttttgtaaatgtaagaaagcatagttattttacaaattgtttttactgtcttttgaagaagttcttaaatacgttgttaaatggtattagttgaccagggcagtgaaaatgaaaccgcattttgggtgccattaaatagggaaaaaacatgtaaaaaatgtaaaatggagaccaattgcactaggcaagtgtatattttgtattttatatacaatttctattatttttcaagtaataaaacaatgtttttcatactgaatattatatatatattttttagctttcatttacttaattattttaagtacctttatttttccaggatgtcagaatttgattctaatctctcttatgtagcacatgtgacttaatttaaaacctatactgtgacacagagttgggtaaacgatgattatttaactttaagcagttcaccatccatttcaaagcctttgattggcttttttgtaaataaaaataacttgttaagaaacaaatatatctgtcatagaagaactagaaaatccagggaagtgagaaaaatgaaaataaaaatcattcatagttttactagtagctaatcacagtcaacctcttttgtgtatcccaccagacttttttatattcatttgtttttagttaaaatataaaagtctcgtatattcccatttttctgcattgcattaccagaaggtagtggcgcctattaaatatgtgatatgttgttgtccagccatggcttctgcatttgcatgcttttgtgtgtgcatctgcaataccctgtgaatatcctgtgtgatggagtggcaagtacgcacagacacgtctgctgcatgcctaggtacgaggctgtctccaggagaagcacttgtttgattatttgagttgccaattgaatttgctgctttttttcatggcttgccattttcactgaaaagaatgactaatgaaaaacgatgattggttattagatttggatgtttggcagacattttctcaaaattgaactaagttggcctcttcacggaaaacaactggtatttgttgtgccaatgataaaattggagatttctagcaaaatgtataattttggaaaagttgtgttcctccactggaagcttgacagctttccttaacataaagacttctctttctcttcgctttcactactactactactaattcttcttctgattcttcttcttctccttcttccttcttccttccttcctcctcctcctccttcttcttcctcttcctcttcttctttctctctttccttccttcccttcccttccccttccttccttccttccttccttcctccctccctccctccctccctccctccctcctttctttttctttctctttctttctttctttctctctctctctctctttctttctttttctttctctttttctttctttcaagcagtcctcccgcctcagtcccccaaaatagtgggattacaggtgtgagccaccatgcacagccttacataaagccttttctaatgagatggatagtaattaacaaatgtgagtttttgatattatataaagattttttctgtgtttcgaagatccgtataactcagtgaatcagtatgttctggatgactaatatgtgatgttaagaaatcatgactgaggccgggcgcggtggctcacgcctgtaatcccagcactttgggaggccgaggcgggcggatcacgagatcaggagatcgagaccaccctggccaacatggtgaaaccccgtctctactaaaaatacaaaaattagctgggtgtgttggtgcgtgcctataatcccagctactcgggaggctgaggcaggagaatcgcttgaactcaggaggcggagattgcagtgagctgagactgcgccactgcaccccagcctggcgacagagcaagactccgtctcaaaaataaaaaaagaaatcatgactgggtaaaagatctgttcagagtacaagatggaccaatggatttgatatatttgaatataacagagtatgaaaaagtttattgatatagtttcagattacacactgcaactaatctttaagaaactattacttgtccactttttggtaaaatttcagagaacaatgtccaccattatctgaacaggctattaaaatactcttctcttttccaactacgtgcctgtgcaaagtcagatttttttcatatacttcagccaaaacagcatatcaaaatggattgaatgcagaagtagatctgagaatacagccacttttgttaagccagacaatgagatttgcaaaatgtaaacaatgctgctgttctcagtttttaaaaatatgttttttaaaagtatttatgttaatgtgtacttggtttactactgctatttttaaataaaacaagaaacatttttaaatgtctgttttaatttctaaagtggtagtgatagatataacccatattaataaaagctctttggggtcctcagtgatttttttttaagagtatggaagggttctcagacctaagagattgagaaatgctgatgtaatgttttattataaaggtgtaccatgaattatgtaccttacttcatattgttggacattaaagttgctttcagtttttttgttttaaa

SEQ ID NO: 296 - DNA, 7575 bp, Homo sapiens
gtgtactgtgtgcgccggggaggcgccggcttgtactcggcagcgcgggaataaagtttgctgatttggtgtctagcctggatgcctgggttgcaggccctgcttgtggtggcgctccacagtcatccggctgaagaagacctgttggactggatcttctcgggttttctttcagatattgttttgtatttacccatgaagacattgttttttggactctgcaaataggacatttcaaagatgagtgaaaaaaaattggaaacaactgcacagcagcggaaatgtcctgaatggatgaatgtgcagaataaaagatgtgctgtagaagaaagaaaggcatgtgttcggaagagtgtttttgaagatgacctccccttcttagaattcactggatccattgtgtatagttacgatgctagtgattgctctttcctgtcagaagatattagcatgagtctatcagatggggatgtggtgggatttgacatggagtggccaccattatacaatagagggaaacttggcaaagttgcactaattcagttgtgtgtttctgagagcaaatgttacttgttccacgtttcttccatgtcagtttttccccagggattaaaaatgttgcttgaaaataaagcagttaaaaaggcaggtgtaggaattgaaggagatcagtggaaacttctacgtgactttgatatcaaattgaagaattttgtggagttgacagatgttgccaataaaaagctgaaatgcacagagacctggagccttaacagtctggttaaacacctcttaggtaaacagctcctgaaagacaagtctatccgctgtagcaattggagtaaatttcctctcactgaggaccagaaactgtatgcagccactgatgcttatgctggttttattatttaccgaaatttagagattttggatgatactgtgcaaaggtttgctataaataaagaggaagaaatcctacttagcgacatgaacaaacagttgacttcaatctctgaggaagtgatggatctggctaagcatcttcctcatgctttcagtaaattggaaaacccacggagggtttctatcttactaaaggatatttcagaaaatctatattcactgaggaggatgataattgggtctactaacattgagactgaactgaggcccagcaataatttaaacttattatcctttgaagattcaactactgggggagtacaacagaaacaaattagagaacatgaagttttaattcacgttgaagatgaaacatgggacccaacacttgatcatttagctaaacatgatggagaagatgtacttggaaataaagtggaacgaaaagaagatggatttgaagatggagtagaagacaacaaattgaaagagaatatggaaagagcttgtttgatgtcgttagatattacagaacatgaactccaaattttggaacagcagtctcaggaagaatatcttagtgatattgcttataaatctactgagcatttatctcccaatgataatgaaaacgatacgtcctatgtaattgagagtgatgaagatttagaaatggagatgcttaagcatttatctcccaatgataatgaaaacgatacgtcctatgtaattgagagtgatgaagatttagaaatggagatgcttaagtctttagaaaacctcaatagtggcacggtagaaccaactcattctaaatgcttaaaaatggaaagaaatctgggtcttcctactaaagaagaagaagaagatgatgaaaatgaagctaatgaaggggaagaagatgatgataaggactttttgtggccagcacccaatgaagagcaagttacttgcctcaagatgtactttggccattccagttttaaaccagttcagtggaaagtgattcattcagtattagaagaaagaagagataatgttgctgtcatggcaactggatatggaaagagtttgtgcttccagtatccacctgtttatgtaggcaagattggccttgttatctctccccttatttctctgatggaagaccaagtgctacagcttaaaatgtccaacatcccagcttgcttccttggatcagcacagtcagaaaatgttctaacagatattaaattaggtaaataccggattgtatacgtaactccagaatactgttcaggtaacatgggcctgctccagcaacttgaggctgatattggtatcacgctcattgctgtggatgaggctcactgtatttctgagtgggggcatgattttagggattcattcaggaagttgggctccctaaagacagcactgccaatggttccaatcgttgcacttactgctactgcaagttcttcaatccgggaagacattgtacgttgcttaaatctgagaaatcctcagatcacctgtactggttttgatcgaccaaacctgtatttagaagttaggcgaaaaacagggaatatccttcaggatctgcagccatttcttgtcaaaacaagttcccactgggaatttgaaggtccaacaatcatctactgtccttctagaaaaatgacacaacaagttacaggtgaacttaggaaactgaatctatcctgtggaacataccatgcgggcatgagttttagcacaaggaaagacattcatcataggtttgtaagagatgaaattcagtgtgtcatagctaccatagcttttggaatgggcattaataaagctgacattcgccaagtcattcattacggtgctcctaaggacatggaatcatattatcaggagattggtagagctggtcgtgatggacttcaaagttcttgtcacgtcctctgggctcctgcagacattaacttaaataggcaccttcttactgagatacgtaatgagaagtttcgattatacaaattaaagatgatggcaaagatggaaaaatatcttcattctagcagatgtaggagacaaatcatcttgtctcattttgaggacaaacaagtacaaaaagcctccttgggaattatgggaactgaaaaatgctgtgataattgcaggtccagattggatcattgctattccatggatgactcagaggatacatcctgggactttggtccacaagcatttaagcttttgtctgctgtggacatcttaggcgaaaaatttggaattgggcttccaattttatttctccgaggatctaattctcagcgtcttgccgatcaatatcgcaggcacagtttatttggcactggcaaggatcaaacagagagttggtggaaggctttttcccgtcagctgatcactgagggattcttggtagaagtttctcggtataacaaatttatgaagatttgcgcccttacgaaaaagggtagaaattggcttcataaagctaatacagaatctcagagcctcatccttcaagctaatgaagaattgtgtccaaagaagttgcttctgcctagttcgaaaactgtatcttcgggcaccaaagagcattgttataatcaagtaccagttgaattaagtacagagaagaagtctaacttggagaagttatattcttataaaccatgtgataagatttcttctgggagtaacatttctaaaaaaagtatcatggtacagtcaccagaaaaagcttacagttcctcacagcctgttatttcggcacaagagcaggagactcagattgtgttatatggcaaattggtagaagctaggcagaaacatgccaataaaatggatgttcccccagctattctggcaacaaacaagatactggtggatatggccaaaatgagaccaactacggttgaaaacgtaaaaaggattgatggtgtttctgaaggcaaagctgccatgttggcccctctgttggaagtcatcaaacatttctgccaaacaaatagtgttcagacagacctcttttcaagtacaaaacctcaagaagaacagaagacgagtctggtagcaaaaaataaaatatgcacactttcacagtctatggccatcacatactctttattccaagaaaagaagatgcctttgaagagcatagctgagagcaggattctgcctctcatgacaattggcatgcacttatcccaagcggtgaaagctggctgcccccttgatttggagcgagcaggcctgactccagaggttcagaagattattgctgatgttatccgaaaccctcccgtcaactcagatatgagtaaaattagcctaatcagaatgttagttcctgaaaacattgacacgtaccttatccacatggcaattgagatccttaaacatggtcctgacagcggacttcaaccttcatgtgatgtcaacaaaaggagatgttttcccggttctgaagagatctgttcaagttctaagagaagcaaggaagaagtaggcatcaatactgagacttcatctgcagagagaaagagacgattacctgtgtggtttgccaaaggaagtgataccagcaagaaattaatggacaaaacgaaaaggggaggtctttttagttaagctggcaattaccagaacaattatgtttcttgctgtattataagaggatagctatattttatttctgaagagtaaggagtagtattttggcttaaaaatcattctaattacaaagttcactgtttattgaagaactggcatcttaaatcagccttccgcaattcatgtagtttctgggtcttctgggagcctacgtgagtacatcacctaacagaatattaaattagacttcctgtaagattgctttaagaaactgttactgtcctgttttctaatctctttattaaaacagtgtatttggaaaatgttatgtgctctgatttgatatagataacagattagtagttacatggtaattatgtgatataaaatattcatatattatcaaaattctgttttgtaaatgtaagaaagcatagttattttacaaattgtttttactgtcttttgaagaagttcttaaatacgttgttaaatggtattagttgaccagggcagtgaaaatgaaaccgcattttgggtgccattaaatagggaaaaaacatgtaaaaaatgtaaaatggagaccaattgcactaggcaagtgtatattttgtattttatatacaatttctattatttttcaagtaataaaacaatgtttttcatactgaatattatatatatattttttagctttcatttacttaattattttaagtacctttatttttccaggatgtcagaatttgattctaatctctcttatgtagcacatgtgacttaatttaaaacctatactgtgacacagagttgggtaaacgatgattatttaactttaagcagttcaccatccatttcaaagcctttgattggcttttttgtaaataaaaataacttgttaagaaacaaatatatctgtcatagaagaactagaaaatccagggaagtgagaaaaatgaaaataaaaatcattcatagttttactagtagctaatcacagtcaacctcttttgtgtatcccaccagacttttttatattcatttgtttttagttaaaatataaaagtctcgtatattcccatttttctgcattgcattaccagaaggtagtggcgcctattaaatatgtgatatgttgttgtccagccatggcttctgcatttgcatgcttttgtgtgtgcatctgcaataccctgtgaatatcctgtgtgatggagtggcaagtacgcacagacacgtctgctgcatgcctaggtacgaggctgtctccaggagaagcacttgtttgattatttgagttgccaattgaatttgctgctttttttcatggcttgccattttcactgaaaagaatgactaatgaaaaacgatgattggttattagatttggatgtttggcagacattttctcaaaattgaactaagttggcctcttcacggaaaacaactggtatttgttgtgccaatgataaaattggagatttctagcaaaatgtataattttggaaaagttgtgttcctccactggaagcttgacagctttccttaacataaagacttctctttctcttcgctttcactactactactactaattcttcttctgattcttcttcttctccttcttccttcttccttccttcctcctcctcctccttcttcttcctcttcctcttcttctttctctctttccttccttcccttcccttccccttccttccttccttccttccttcctccctccctccctccctccctccctccctcctttctttttctttctctttctttctttctttctctctctctctctctttctttctttttctttctctttttctttctttcaagcagtcctcccgcctcagtcccccaaaatagtgggattacaggtgtgagccaccatgcacagccttacataaagccttttctaatgagatggatagtaattaacaaatgtgagtttttgatattatataaagattttttctgtgtttcgaagatccgtataactcagtgaatcagtatgttctggatgactaatatgtgatgttaagaaatcatgactgaggccgggcgcggtggctcacgcctgtaatcccagcactttgggaggccgaggcgggcggatcacgagatcaggagatcgagaccaccctggccaacatggtgaaaccccgtctctactaaaaatacaaaaattagctgggtgtgttggtgcgtgcctataatcccagctactcgggaggctgaggcaggagaatcgcttgaactcaggaggcggagattgcagtgagctgagactgcgccactgcaccccagcctggcgacagagcaagactccgtctcaaaaataaaaaaagaaatcatgactgggtaaaagatctgttcagagtacaagatggaccaatggatttgatatatttgaatataacagagtatgaaaaagtttattgatatagtttcagattacacactgcaactaatctttaagaaactattacttgtccactttttggtaaaatttcagagaacaatgtccaccattatctgaacaggctattaaaatactcttctcttttccaactacgtgcctgtgcaaagtcagatttttttcatatacttcagccaaaacagcatatcaaaatggattgaatgcagaagtagatctgagaatacagccacttttgttaagccagacaatgagatttgcaaaatgtaaacaatgctgctgttctcagtttttaaaaatatgttttttaaaagtatttatgttaatgtgtacttggtttactactgctatttttaaataaaacaagaaacatttttaaatgtctgttttaatttctaaagtggtagtgatagatataacccatattaataaaagctctttggggtcctcagtgatttttttttaagagtatggaagggttctcagacctaagagattgagaaatgctgatgtaatgttttattataaaggtgtaccatgaattatgtaccttacttcatattgttggacattaaagttgctttcagtttttttgttttaaa

SEQ ID NO: 297 - DNA, 4222 bp, Homo sapiens
gtgtactgtgtgcgccggggaggcgccggcttgtactcggcagcgcgggaataaagtttgctgatttggtgtctagcctggatgcctgggttgcaggccctgcttgtggtggcgctccacagtcatccggctgaagaagacctgttggactggatcttctcgggttttctttcagatattgttttgtatttacccatgaagacattgttttttggactctgcaaataggacatttcaaagatgagtgaaaaaaaattggaaacaactgcacagcagcggaaatgtcctgaatggatgaatgtgcagaataaaagatgtgctgtagaagaaagaaaggcatgtgttcggaagagtgtttttgaagatgacctccccttcttagaattcactggatccattgtgtatagttacgatgctagtgattgctctttcctgtcagaagatattagcatgagtctatcagatggggatgtggtgggatttgacatggagtggccaccattatacaatagagggaaacttggcaaagttgcactaattcagttgtgtgtttctgagagcaaatgttacttgttccacgtttcttccatgtcagtttttccccagggattaaaaatgttgcttgaaaataaagcagttaaaaaggcaggtgtaggaattgaaggagatcagtggaaacttctacgtgactttgatatcaaattgaagaattttgtggagttgacagatgttgccaataaaaagctgaaatgcacagagacctggagccttaacagtctggttaaacacctcttaggtaaacagctcctgaaagacaagtctatccgctgtagcaattggagtaaatttcctctcactgaggaccagaaactgtatgcagccactgatgcttatgctggttttattatttaccgaaatttagagattttggatgatactgtgcaaaggtttgctataaataaagaggaagaaatcctacttagcgacatgaacaaacagttgacttcaatctctgaggaagtgatggatctggctaagcatcttcctcatgctttcagtaaattggaaaacccacggagggtttctatcttactaaaggatatttcagaaaatctatattcactgaggaggatgataattgggtctactaacattgagactgaactgaggcccagcaataatttaaacttattatcctttgaagattcaactactgggggagtacaacagaaacaaattagagaacatgaagttttaattcacgttgaagatgaaacatgggacccaacacttgatcatttagctaaacatgatggagaagatgtacttggaaataaagtggaacgaaaagaagatggatttgaagatggagtagaagacaacaaattgaaagagaatatggaaagagcttgtttgatgtcgttagatattacagaacatgaactccaaattttggaacagcagtctcaggaagaatatcttagtgatattgcttataaatctactgagcatttatctcccaatgataatgaaaacgatacgtcctatgtaattgagagtgatgaagatttagaaatggagatgcttaagcatttatctcccaatgataatgaaaacgatacgtcctatgtaattgagagtgatgaagatttagaaatggagatgcttaagtctttagaaaacctcaatagtggcacggtagaaccaactcattctaaatgcttaaaaatggaaagaaatctgggtcttcctactaaagaagaagaagaagatgatgaaaatgaagctaatgaaggggaagaagatgatgataaggactttttgtggccagcacccaatgaagagcaagttacttgcctcaagatgtactttggccattccagttttaaaccagttcagtggaaagtgattcattcagtattagaagaaagaagagataatgttgctgtcatggcaactggatatggaaagagtttgtgcttccagtatccacctgtttatgtaggcaagattggccttgttatctctccccttatttctctgatggaagaccaagtgctacagcttaaaatgtccaacatcccagcttgcttccttggatcagcacagtcagaaaatgttctaacagatattaaattaggtaaataccggattgtatacgtaactccagaatactgttcaggtaacatgggcctgctccagcaacttgaggctgatattggtatcacgctcattgctgtggatgaggctcactgtatttctgagtgggggcatgattttagggattcattcaggaagttgggctccctaaagacagcactgccaatggttccaatcgttgcacttactgctactgcaagttcttcaatccgggaagacattgtacgttgcttaaatctgagaaatcctcagatcacctgtactggttttgatcgaccaaacctgtatttagaagttaggcgaaaaacagggaatatccttcaggatctgcagccatttcttgtcaaaacaagttcccactgggaatttgaaggtccaacaatcatctactgtccttctagaaaaatgacacaacaagttacaggtgaacttaggaaactgaatctatcctgtggaacataccatgcgggcatgagttttagcacaaggaaagacattcatcataggtttgtaagagatgaaattcagtgtgtcatagctaccatagcttttggaatgggcattaataaagctgacattcgccaagtcattcattacggtgctcctaaggacatggaatcatattatcaggagattggtagagctggtcgtgatggacttcaaagttcttgtcacgtcctctgggctcctgcagacattaacttaaataggcaccttcttactgagatacgtaatgagaagtttcgattatacaaattaaagatgatggcaaagatggaaaaatatcttcattctagcagatgtaggagacaaatcatcttgtctcattttgaggacaaacaagtacaaaaagcctccttgggaattatgggaactgaaaaatgctgtgataattgcaggtccagattggatcattgctattccatggatgactcagaggatacatcctgggactttggtccacaagcatttaagcttttgtctgctgtggacatcttaggcgaaaaatttggaattgggcttccaattttatttctccgaggatctaattctcagcgtcttgccgatcaatatcgcaggcacagtttatttggcactggcaaggatcaaacagagagttggtggaaggctttttcccgtcagctgatcactgagggattcttggtagaagtttctcggtataacaaatttatgaagatttgcgcccttacgaaaaagggtagaaattggcttcataaagctaatacagaatctcagagcctcatccttcaagctaatgaagaattgtgtccaaagaagttgcttctgcctagttcgaaaactgtatcttcgggcaccaaagagcattgttataatcaagtaccagttgaattaagtacagagaagaagtctaacttggagaagttatattcttataaaccatgtgataagatttcttctgggagtaacatttctaaaaaaagtatcatggtacagtcaccagaaaaagcttacagttcctcacagcctgttatttcggcacaagagcaggagactcagattgtgttatatggcaaattggtagaagctaggcagaaacatgccaataaaatggatgttcccccagctattctggcaacaaacaagatactggtggatatggccaaaatgagaccaactacggttgaaaacgtaaaaaggattgatggtgtttctgaaggcaaagctgccatgttggcccctctgttggaagtcatcaaacatttctgccaaacaaatagtgttcagacagacctcttttcaagtacaaaacctcaagaagaacagaagacgagtctggtagcaaaaaataaaatatgcacactttcacagtctatggccatcacatactctttattccaagaaaagaagatgcctttgaagagcatagctgagagcaggattctgcctctcatgacaattggcatgcacttatcccaagcggtgaaagctggctgcccccttgatttggagcgagcaggcctgactccagaggttcagaagattattgctgatgttatccgaaaccctcccgtcaactcag

SEQ ID NO: 298 - DNA, 150 bp, Homo sapiens
gaaggaaactgtttaaccggatcccattgtacccagagtgcagagccgcctttccagcatgcaggggctgctcaggacagagagggaggaggcgcgcggggacggggacgcccaggaggacccactcgcgggtcccgctccgctccggca

SEQ ID NO: 299 - DNA, 102 bp, Homo sapiens
gcgagttagtaacctacgagcggctgtgaaggaaactgtttaaccggatcccattgtacccagagtgcagagccgcctttccagcatgcaggggctgctcag

SEQ ID NO: 300 - DNA, 220 bp, Homo sapiens
cgtttagtcacatcaagaaatagaacagaattcagccatggccccaagaaagagaggtggacgaggtatttcattcatcttttgctgtttccgaaataatgatcacccagaaatcacgtatcggctgcgaaatgatagcaactttgcgcttcagaccatggaaccagcattgcccatgccccctgtggaggagctggatgtcatgttcagtgaactggtg

SEQ ID NO: 301 - DNA, 90 bp, Homo sapiens
gatgaactggacctcacagacaaacacagagaagccatgtttgcacttccagctgagaaaaaatggcaaatatactgtagcaagaaaaag

SEQ ID NO: 302 - DNA, 72 bp, Homo sapiens
gaccaggaagaaaacaagggagctacaagttggcctgaattctacattgatcagctcaattccatggctgct

SEQ ID NO: 303 - DNA, 95 bp, Homo sapiens
agaaaatctctgctggctttagagaaggaagaagaagaagaaagaagtaaaactatagagagtttaaagacagcactgaggacaaaaccaatgag

SEQ ID NO: 304 - DNA, 334 bp, Homo sapiens
gtttgtaaccagattcatcgacttggatggcctatcatgtatcctcaactttctaaagaccatggactacgagacctcagagtctcgaatacatacttctctcattggctgtataaaggcgttaatgaacaactctcaaggccgggctcacgtcctggctcattctgagagtattaatgtaattgctcagagtctgagcacagagaacattaaaacgaaggtggccgtgctggaaatcttgggcgccgtgtgcctggttcccgggggccacaagaaggttctgcaggccatgctgcactaccagaagtatgccagcgaaaggacccgctttcag

SEQ ID NO: 305 - DNA, 111 bp, Homo sapiens
acattaattaacgacttggataaaagcactgggcggtatcgagatgaagtgagtctcaagactgccatcatgtccttcattaatgcagtgctcagccaaggtgcaggagtg

SEQ ID NO: 306 - DNA, 104 bp, Homo sapiens
gagagtttggactttagacttcatcttcgctatgaatttctgatgttaggaattcaacctgtaatagataaattaagggaacacgaaaattcaacattagatag

SEQ ID NO: 307 - DNA, 67 bp, Homo sapiens
gcatttagacttttttgaaatgctccgaaatgaagatgaactagaatttgccaaaagatttgaactg

SEQ ID NO: 308 - DNA, 118 bp, Homo sapiens
gttcacatagacacaaaaagtgcaactcagatgtttgagctgaccaggaagaggctgacacatagtgaagcttacccgcatttcatgtccatcctgcaccactgcctccaaatgcctt

SEQ ID NO: 309 - DNA, 139 bp, Homo sapiens
acaagaggagtggcaacactgttcagtactggctactactagatagaattatacagcagatagttatccagaatgacaaaggacaggaccctgactccacacctttggaaaactttaatattaagaatgtcgtacgaat

SEQ ID NO: 310 - DNA, 59 bp, Homo sapiens
gttggttaatgaaaatgaagttaagcagtggaaagaacaagcggaaaaaatgagaaaag

SEQ ID NO: 311 - DNA, 188 bp, Homo sapiens
agcacaatgagctacaacagaaactggaaaagaaagaacgagaatgtgatgctaagactcaagagaaggaagagatgatgcagaccttaaataaaatgaaagagaaacttgaaaaggagactactgagcataagcaagtcaagcagcaggtggcggacctcacagcacagctccatgagctcagcagg

SEQ ID NO: 312 - DNA, 300 bp, Homo sapiens
agggccgtctgtgcttcaatcccaggtggaccctcgcctggagcaccaggagggccctttccttcctctgtgcctggatctctccttcctcccccaccacccccacctctaccaggtgggatgcttccccctccaccgcctcccctccctccaggtggccctcctcctcccccagggcctcctcccttaggggcaatcatgccacctcctggtgctccaatgggcctagcactgaagaagaaaagcattcctcagcccacaaatgccctgaaatccttcaactggtctaaactgcccgag

SEQ ID NO: 313 - DNA, 108 bp, Homo sapiens
aacaaactggaaggaacagtatggaccgaaattgatgatacaaaagtcttcaaaattctagatcttgaagacctggaaagaaccttctctgcctatcaaagacagcag

SEQ ID NO: 314 - DNA, 107 bp, Homo sapiens
aaagaagcagatgccattgatgacactctgagttccaaacttaaagttaaagagctttcggtgattgatggtcggagagctcagaattgcaacatccttctatcgag

SEQ ID NO: 315 - DNA, 85 bp, Homo sapiens
gttgaaattatccaatgacgaaatcaaacgggcaattctaacaatggacgaacaggaagatctgcccaaggacatgttggaacag

SEQ ID NO: 316 - DNA, 107 bp, Homo sapiens
ctcttgaaatttgttcctgaaaaaagtgacattgacctattggaggaacataaacacgaactggatcggatggccaaggctgataggttcctttttgagatgagccg

SEQ ID NO: 317 - DNA, 89 bp, Homo sapiens
aattaatcactatcagcaaaggttgcaatcgctgtacttcaaaaagaagtttgcagagcgtgtggcagaagtgaaacctaaagtggaag

SEQ ID NO: 318 - DNA, 169 bp, Homo sapiens
caattcgttctggctcagaagaggtgtttaggagtggtgccctcaagcagttgctggaggtggttttggcatttggaaattatatgaataaaggtcaaagagggaatgcatatggattcaagatatctagcctaaacaaaattgctgacacaaaatccagcatcgacaa

SEQ ID NO: 319 - DNA, 108 bp, Homo sapiens
aaacattacccttttgcactatctcatcactattgtggaaaataagtaccccagtgttctcaatctaaatgaagaattgcgagatattcctcaagctgcgaaagtaaa

SEQ ID NO: 320 - DNA, 61 bp, Homo sapiens
catgactgagctggacaaagaaataagtaccttgagaagtggcttgaaagcagtagagaca

SEQ ID NO: 321 - DNA, 132 bp, Homo sapiens
gagctggaatatcagaagtctcagcccccacagcccggagataagtttgtgtctgttgtcagccagttcatcacagtagccagcttcagcttctctgatgttgaagaccttctagcagaagctaaagacctg

SEQ ID NO: 322 - DNA, 171 bp, Homo sapiens
tttactaaagcagtgaagcactttggggaagaggctggcaaaatacaaccagatgagttctttggcatttttgatcaatttcttcaagctgtgtcagaagccaaacaagaaaacgaaaatatgagaaagaaaaaggaggaagaagaacgtcgagctcgcatggaagctcag

SEQ ID NO: 323 - DNA, 5890 bp, Homo sapiens
gcgagttagtaacctacgagcggctgtgaaggaaactgtttaaccggatcccattgtacccagagtgcagagccgcctttccagcatgcaggggctgctcagcgtttagtcacatcaagaaatagaacagaattcagccatggccccaagaaagagaggtggacgaggtatttcattcatcttttgctgtttccgaaataatgatcacccagaaatcacgtatcggctgcgaaatgatagcaactttgcgcttcagaccatggaaccagcattgcccatgccccctgtggaggagctggatgtcatgttcagtgaactggtggatgaactggacctcacagacaaacacagagaagccatgtttgcacttccagctgagaaaaaatggcaaatatactgtagcaagaaaaaggaccaggaagaaaacaagggagctacaagttggcctgaattctacattgatcagctcaattccatggctgctagaaaatctctgctggctttagagaaggaagaagaagaagaaagaagtaaaactatagagagtttaaagacagcactgaggacaaaaccaatgaggtttgtaaccagattcatcgacttggatggcctatcatgtatcctcaactttctaaagaccatggactacgagacctcagagtctcgaatacatacttctctcattggctgtataaaggcgttaatgaacaactctcaaggccgggctcacgtcctggctcattctgagagtattaatgtaattgctcagagtctgagcacagagaacattaaaacgaaggtggccgtgctggaaatcttgggcgccgtgtgcctggttcccgggggccacaagaaggttctgcaggccatgctgcactaccagaagtatgccagcgaaaggacccgctttcagacattaattaacgacttggataaaagcactgggcggtatcgagatgaagtgagtctcaagactgccatcatgtccttcattaatgcagtgctcagccaaggtgcaggagtggagagtttggactttagacttcatcttcgctatgaatttctgatgttaggaattcaacctgtaatagataaattaagggaacacgaaaattcaacattagataggcatttagacttttttgaaatgctccgaaatgaagatgaactagaatttgccaaaagatttgaactggttcacatagacacaaaaagtgcaactcagatgtttgagctgaccaggaagaggctgacacatagtgaagcttacccgcatttcatgtccatcctgcaccactgcctccaaatgccttacaagaggagtggcaacactgttcagtactggctactactagatagaattatacagcagatagttatccagaatgacaaaggacaggaccctgactccacacctttggaaaactttaatattaagaatgtcgtacgaatgttggttaatgaaaatgaagttaagcagtggaaagaacaagcggaaaaaatgagaaaagagcacaatgagctacaacagaaactggaaaagaaagaacgagaatgtgatgctaagactcaagagaaggaagagatgatgcagaccttaaataaaatgaaagagaaacttgaaaaggagactactgagcataagcaagtcaagcagcaggtggcggacctcacagcacagctccatgagctcagcaggagggccgtctgtgcttcaatcccaggtggaccctcgcctggagcaccaggagggccctttccttcctctgtgcctggatctctccttcctcccccaccacccccacctctaccaggtgggatgcttccccctccaccgcctcccctccctccaggtggccctcctcctcccccagggcctcctcccttaggggcaatcatgccacctcctggtgctccaatgggcctagcactgaagaagaaaagcattcctcagcccacaaatgccctgaaatccttcaactggtctaaactgcccgagaacaaactggaaggaacagtatggaccgaaattgatgatacaaaagtcttcaaaattctagatcttgaagacctggaaagaaccttctctgcctatcaaagacagcagaaagaagcagatgccattgatgacactctgagttccaaacttaaagttaaagagctttcggtgattgatggtcggagagctcagaattgcaacatccttctatcgaggttgaaattatccaatgacgaaatcaaacgggcaattctaacaatggacgaacaggaagatctgcccaaggacatgttggaacagctcttgaaatttgttcctgaaaaaagtgacattgacctattggaggaacataaacacgaactggatcggatggccaaggctgataggttcctttttgagatgagccgaattaatcactatcagcaaaggttgcaatcgctgtacttcaaaaagaagtttgcagagcgtgtggcagaagtgaaacctaaagtggaagcaattcgttctggctcagaagaggtgtttaggagtggtgccctcaagcagttgctggaggtggttttggcatttggaaattatatgaataaaggtcaaagagggaatgcatatggattcaagatatctagcctaaacaaaattgctgacacaaaatccagcatcgacaaaaacattacccttttgcactatctcatcactattgtggaaaataagtaccccagtgttctcaatctaaatgaagaattgcgagatattcctcaagctgcgaaagtaaacatgactgagctggacaaagaaataagtaccttgagaagtggcttgaaagcagtagagacagagctggaatatcagaagtctcagcccccacagcccggagataagtttgtgtctgttgtcagccagttcatcacagtagccagcttcagcttctctgatgttgaagaccttctagcagaagctaaagacctgtttactaaagcagtgaagcactttggggaagaggctggcaaaatacaaccagatgagttctttggcatttttgatcaatttcttcaagctgtgtcagaagccaaacaagaaaacgaaaatatgagaaagaaaaaggaggaagaagaacgtcgagctcgcatggaagctcagctcaaagaacaacgtgaaagggaacgtaaaatgagaaaagctaaagagaatagtgaagaaagcggagagtttgatgaccttgtttcagctttacgctcaggagaagtgtttgacaaagacctttctaaattgaaacggaatcgcaaacgtattaccaaccagatgactgacagcagcagagagagaccaatcacaaaacttaatttctaattttccatgaatacttttttttagaaagctcattagcagccctctaaagtgactagaacgtttcattacactgccttgcaatccaaacagtggcaattttttccttcatctgtgagtgaatgtgtgaacgtgtgtatgtaaatgtatgtgtgtatatattaaaaaatgtatatagatgtctgagtgttgtctggagacctatacgtatggttaaaaagatttatgttaatgtatgtgctccaaaacctttcgtgtatgcattcacattgagtgtggctcattttctttccccgaacgccatgactgttcagaagcacaatactatctcctgaaagagataagagacattccctagattcaaaggcaaaacagaagaaacaaacaaacaaacaaaaaaagcttgcaaaatattttatggtttccaagcttgatatcctttaaaattattttcattgatggaactggagttgttggaaaaacatagatttaaaatgatttttgatagctgacattgtgatgttgatgtatcacatcagtaataggaccagctttgaatttctgacattggtgtggggatacagtctgtaaatgtttattgagaacatcttgcacacaatttgaattatgtagaatgtcaatcaagtttttgtatatttaaaagttggacatcaattttttcccctgatttcatcaagttatctctgccaagtgctcttgataatttcttcagatttttggaaaaaaacactatataaatgcaatccatgctttttttaaagaacaacattgccagagtatgcttgttctaacaatatagatatataaaccttaaaaataataaaatatctcacccaagacttaaaggaagaattctctgaagggataaagattactaaaaaaaaaaaaaaaaaaaaaaaattaatggggtgcctttttgttatagtttctattttctgttttgtaggacaagctgcattttctgtaaatataggtctggactaaaggatacataaagaatgcacaaaatgtcaacatcagcagagatgcccagatctatttatctctaagtatatttgaagtgattgctgtttatatgttgtcattttaaaattgtgtgtcagtaaagctacctgtaaaatttcagtccaaaaaaataaagctctcagggagacatgaataaaatcaatgaacattagaaaataaaatatagatgcttaccattaacctaccaactcttaatatccttaaattatgtgatatataaagaggactgttacttttttacttttttttttttttttttttttttttggctttgctttatttatttgtagttgggggctaacgttttctcttttctttctattgatcctgttgtggttgggtttcctgtggagagagtagtttgtcctgttgcactagaacattatttactcactaaattgagtttttcagtcaattaacaaatatttattaagttcctactatgtaccaggcatagtagggctacaatggtaagcaagacagagtccctgcccccaaagagcttatattctaatggggatattaagggatgaatagaataccaatgtgtgcactgtacaggaatcatactatttaaaaataatttgtataaactataatgcttagcacagatggcgagttatctgtgctatgtgaaagctgtgaaatagtgctctaagagttgtcaagagctgtggttttacatcttttcctcattgcaaatttagtgactttctacacactatatggaaataaatgactagcaaataaaacagtcataaatacaaagcagaggttgcactcccccaatcccgagttaacccaggtctgcaataacaccatgttaaaggtgcagatagagacttggctcaaaaaggcttggagatgaggaagattggaatataatgatggttttgtctgttccttaactaaagtgcctctatgtatattcttttctatttgtagcaggataaatttggtctggctcagttttggaactgtattttgaaaatggctttgtcttacagtttaaggaatagacaggtggagggaaagtcacataaaggagcaagtttgtgtagctgtccctcttgccccttttaatcatcctcctttgatatggccatcctggtgggcctcctttgccatttccatttttggtttctttccctgaaaactgtgtgcaggtaattccatgtgccattgttgaaaagaaaaaaaaaaaacaaaaaaaaaacctactttttagattggtgctggtgtaagtagccacttttctctcttgggtgtgtattttaaactttttttgtttttttaaattaatgccaaaaagaaaatgcataatttgtaaacttaattatatgtcttatatcttattagcttagtagttggaaccacttagtctttaggtgcaagactgttgttagatagtactgagaaaaaaaaagtatgtgttatgagactgtacatgtttttttaaaaatagcaatatgcaataaagagatgaattcattgggtgta

SEQ ID NO: 324 - DNA, 5890 bp, Homo sapiens; identical to SEQ ID NO: 323

SEQ ID NO: 325 - DNA, 150 bp, Homo sapiens
aaggtctcttcctgcctgaggatgaaaacctgagggaaaaaggggactggagccagttcacgctgtggcagcaagccttgcctccccgattgaaagagatgaaaagccaggaatcggctgcaggttccaaactagtccttcggtgtgaaa

SEQ ID NO: 326 - DNA, 139 bp, Homo sapiens
agtctcaagctctggtaggcaagtgcatgcagtgtgcctaaaacctgccagcagtacttttgagtttttttttttgttttgttttactttagcatttattattcatggattgaagaaatcaaaatggctgaagataaag

SEQ ID NO: 327 - DNA, 150 bp, Homo sapiens
agacaaagcatggaggacacaagaatgggaggaaaggcggactctcaggaacttcattcttcacgtggtttatggtgattgcattgctgggcgtctggacatctgtagctgtcgtttggtttgatcttgttgactatgaggaagttctag

SEQ ID NO: 328 - DNA, 69 bp, Homo sapiens
gaaaactaggaatctatgatgctgatggtgatggagattttgatgtggatgatgccaaagttttattag

SEQ ID NO: 329 - DNA, 93 bp, Homo sapiens
gacttaaagagagatctacttcagagccagcagtcccgccagaagaggctgagccacacactgagcccgaggagcaggttcctgtggaggcag

SEQ ID NO: 330 - DNA, 75 bp, Homo sapiens
aaccccagaatatcgaagatgaagcaaaagaacaaattcagtcccttctccatgaaatggtacacgcagaacatg

SEQ ID NO: 331 - DNA, 129 bp, Homo sapiens
ttgagggagaagacttgcaacaagaagatggacccacaggagaaccacaacaagaggatgatgagtttcttatggcgactgatgtagatgatagatttgagaccctggaacctgaagtatctcatgaag

SEQ ID NO: 332 - DNA, 33 bp, Homo sapiens
aaaccgagcatagttaccacgtggaagagacag

SEQ ID NO: 333 - DNA, 57 bp, Homo sapiens
tttcacaagactgtaatcaggatatggaagagatgatgtctgagcaggaaaatccag

SEQ ID NO: 334 - DNA, 48 bp, Homo sapiens
attccagtgaaccagtagtagaagatgaaagattgcaccatgatacag

SEQ ID NO: 335 - DNA, 33 bp, Homo sapiens
atgatgtaacataccaagtctatgaggaacaag

SEQ ID NO: 336 - DNA, 42 bp, Homo sapiens
cagtatatgaacctctagaaaatgaagggatagaaatcacag

SEQ ID NO: 337 - DNA, 57 bp, Homo sapiens
aagtaactgctccccctgaggataatcctgtagaagattcacaggtaattgtagaag

SEQ ID NO: 338 - DNA, 45 bp, Homo sapiens
aagtaagcatttttcctgtggaagaacagcaggaagtaccaccag

SEQ ID NO: 339 - DNA, 42 bp, Homo sapiens
aaacaaatagaaaaacagatgatccagaacaaaaagcaaaag

SEQ ID NO: 340 - DNA, 86 bp, Homo sapiens
ttaagaaaaagaagcctaaacttttaaataaatttgataagactattaaagctgaacttgatgctgcagaaaaactccgtaaaagg

SEQ ID NO: 341 - DNA, 87 bp, Homo sapiens
ggaaaaattgaggaagcagtgaatgcatttaaagaactagtacgcaaataccctcagagtccacgagcaagatatgggaaggcgcag

SEQ ID NO: 342 - DNA, 151 bp, Homo sapiens
tgtgaggatgatttggctgagaagaggagaagtaatgaggtgctacgtggagccatcgagacctaccaagaggtggccagcctacctgatgtccctgcagacctgctgaagctgagtttgaagcgtcgctcagacaggcaacaatttctag

SEQ ID NO: 343 - DNA, 137 bp, Homo sapiens
gtcatatgagaggttccctgcttaccctgcagagattagttcaactatttcccaatgatacttccttaaaaaatgaccttggcgtgggatacctcttgataggagataatgacaatgcaaagaaagtttatgaagag

SEQ ID NO: 344 - DNA, 99 bp, Homo sapiens
gtgctgagtgtgacacctaatgatggctttgctaaagtccattatggcttcatcctgaaggcacagaacaaaattgctgagagcatcccatatttaaag

SEQ ID NO: 345 - DNA, 90 bp, Homo sapiens
gaaggaatagaatccggagatcctggcactgatgatgggagattttatttccacctgggggatgccatgcagagggttgggaacaaagag

SEQ ID NO: 346 - DNA, 138 bp, Homo sapiens
gcatataagtggtatgagcttgggcacaagagaggacactttgcatctgtctggcaacgctcactctacaatgtgaatggactgaaagcacagccttggtggaccccaaaagaaacgggctacacagagttagtaaag

SEQ ID NO: 347 - DNA, 136 bp, Homo sapiens
tctttagaaagaaactggaagttaatccgagatgaaggccttgcagtgatggataaagccaaaggtctcttcctgcctgaggatgaaaacctgagggaaaaaggggactggagccagttcacgctgtggcagcaag

SEQ ID NO: 348 - DNA, 92 bp, Homo sapiens
gaagaagaaatgaaaatgcctgcaaaggagctcctaaaacctgtaccttactagaaaagttccccgagacaacaggatgcagaagaggacag

SEQ ID NO: 349 - DNA, 134 bp, Homo sapiens
atcaaatattccatcatgcaccccgggactcacgtgtggccgcacacagggcccacaaactgcaggctccgaatgcacctgggcttggtgattcccaaggaaggctgcaagattcgatgtgccaacgagaccaa

SEQ ID NO: 350 - DNA, 2954 bp, Homo sapiens
gacctgggaggaaggcaaggtgctcatctttgatgactcctttgagcacgaggtatggcaggatgcctcatctttccggctgatattcatcgtggatgtgtggcatccggaactgacaccacagcagagacgcagccttccagcaatttagcatgaattcatgcaagcttgggaaactctggagagaggctgcctttctggttccatctccttgggtgtgaggatagaatttcgaacaccaagagtcaattcccttgacttgcagcccgagtaattcaaagcctcctcctagggtcagaagacactaaagggaatatttgcctcgctgcaattcatttaggaaacaccctgctgtgtgtcatctcatgacagcactggtcttctgccagtatttaaggtgaacatttgatagcttctaccttaccagccaaagatattttttccacatagaataggtctaattcaatgtataatgagaacatatgtagaaactgtgaatggattgctttagtttgtaatttttctatgcagttatatttttctagtgtagctagactattttgtcatcatgtaccactacatttttgtttattttaatgacaagctgtataaatgctttacttctagctatttaatggtagcattactggggaactcagacttccctcttttaattcttcttagtaaaagatactcatgaaaaaagcagttttattttcctaacaaaaaagaaagagctcattatgtcagtgtctatgaactgtacccatcccaactctcaaatcgtttggttttttttatcttgattgagatcctcttctcactatgctagtggtggagatattgacaaaatcctatttctttcaaagaggaacttttcacaccgaaaaaagagcatggaattattttatattgttataaaaatcccagatgcaaatttttttaatgccaattattagagcttctggggaaaaagtatagttcacggaaataaaactatgttctttcagggttgggtggataggtggctgctagggtgtctggctcctggcggctttgccatccatgaggcaagggctgggaacacagtgtctttgcctatggtagatccatgtgaatgtcaggaagccagctcttcagtcttggagatgatttctgctacaattctgtagaaagattaaggatggcagagtaaaaggttaccaagaatgccaggatgtttttcttgggcgtaggaggtccagattactttcctttttgatgaaagagtttggaagactgtcccatctctctggcttgagaaatctctgccattttaaacatcactgtgaaatagcaattattatcatctgtatttagttttaacattacccacaacatagaaataataggtaaaaatcgtcttgcctactcattccaaagatgatcaagtcattaatctagcaaagtattcatgtatcagattttgtatattttgaatcaaagctaactaggaatgttagatataagaatgtaatgatattcatgcactgaattctaagccaatatgaacaaaaatgctgcatgaatggcacatataggtcaccaaagttcattcacaggtagaaaaaacttgtgctttcttttccatctaaaaacaaaaggagactttctttatctcatttaaagaacagctctttgaaattgaaattgaccctttttgcttgaccttaaggagattagcttccagtagatgagtttgcaaaatacttttcctgttcttttgttttgctggtattgaaaacatcccactaaatcagatgaagaggcatgggaggaaaaatatccaaattaattactaaaatcgagaagagaaggcaaactcttgaaaagtaaaaaggtgtttgtgaccttcagtatttattgaacagaggaaataactgacaagggcaatacaattcaatgttcatgtagtaacattcatgtcacttgttgaatttggttctcatatgtatattgcatacacataaattcaaactataagtcgtcatttttgagccatcatcttacattcatgtaatgaaattatggaagagagtaaaaactagctcttaacttagtaaatataatatggtatttaaaatcaggtcactacagtaaggttctaagtattgccaattgaaaagctagaaatggtattactgttgcaaagtgttgtcaataattgactccaatagcattgtaaatacttgtatcccacaactattttaaacccaagcaataaaatggattttctaattcacttcaattctttatttctcttacctatctatgtcttggtacataagaaaagtgtttccatcactgcattggtagaattatttcagtgttattatttttgtgatttcgtatgtctacacaaaaatgaattagtttaatttatattgtgatacagttgtttgagaaatatttttaattctgtttcaactttatttctccaagtgtataatataaaaattacttctgttattgttctctaccaataggggaaaaaattaaaacattagatccattgagaaagagatgatgtaataaataattaagattagtaataatattatcaggggtgattatgaccagttgaataatctctttcccttgaattatttagctaacaaattaactctcccaaatatttaaaataatgtaaaatcatattttactgcccattattaactaaaatatttttgtttgactttgagcaccaactggtaatactaataaatacccatgtcatgcagatggctgggcgaataagagatgtctaaaaatatgcactggtcttggaaaacatggcacaagtaaggatatcatatatgatgtctgtttattttatgtctgatttcttttgaatgagtagttggggactccatttctaaggagactaggtaaataaaatgacctttgacatttca

SEQ ID NO: 351 - DNA, 5116 bp, Homo sapiens
agtctcaagctctggtaggcaagtgcatgcagtgtgcctaaaacctgccagcagtacttttgagtttttttttttgttttgttttactttagcatttattattcatggattgaagaaatcaaaatggctgaagataaagagacaaagcatggaggacacaagaatgggaggaaaggcggactctcaggaacttcattcttcacgtggtttatggtgattgcattgctgggcgtctggacatctgtagctgtcgtttggtttgatcttgttgactatgaggaagttctaggaaaactaggaatctatgatgctgatggtgatggagattttgatgtggatgatgccaaagttttattaggacttaaagagagatctacttcagagccagcagtcccgccagaagaggctgagccacacactgagcccgaggagcaggttcctgtggaggcagaaccccagaatatcgaagatgaagcaaaagaacaaattcagtcccttctccatgaaatggtacacgcagaacatgttgagggagaagacttgcaacaagaagatggacccacaggagaaccacaacaagaggatgatgagtttcttatggcgactgatgtagatgatagatttgagaccctggaacctgaagtatctcatgaagaaaccgagcatagttaccacgtggaagagacagtttcacaagactgtaatcaggatatggaagagatgatgtctgagcaggaaaatccagattccagtgaaccagtagtagaagatgaaagattgcaccatgatacagatgatgtaacataccaagtctatgaggaacaagcagtatatgaacctctagaaaatgaagggatagaaatcacagaagtaactgctccccctgaggataatcctgtagaagattcacaggtaattgtagaagaagtaagcatttttcctgtggaagaacagcaggaagtaccaccagaaacaaatagaaaaacagatgatccagaacaaaaagcaaaagttaagaaaaagaagcctaaacttttaaataaatttgataagactattaaagctgaacttgatgctgcagaaaaactccgtaaaaggggaaaaattgaggaagcagtgaatgcatttaaagaactagtacgcaaataccctcagagtccacgagcaagatatgggaaggcgcagtgtgaggatgatttggctgagaagaggagaagtaatgaggtgctacgtggagccatcgagacctaccaagaggtggccagcctacctgatgtccctgcagacctgctgaagctgagtttgaagcgtcgctcagacaggcaacaatttctaggtcatatgagaggttccctgcttaccctgcagagattagttcaactatttcccaatgatacttccttaaaaaatgaccttggcgtgggatacctcttgataggagataatgacaatgcaaagaaagtttatgaagaggtgctgagtgtgacacctaatgatggctttgctaaagtccattatggcttcatcctgaaggcacagaacaaaattgctgagagcatcccatatttaaaggaaggaatagaatccggagatcctggcactgatgatgggagattttatttccacctgggggatgccatgcagagggttgggaacaaagaggcatataagtggtatgagcttgggcacaagagaggacactttgcatctgtctggcaacgctcactctacaatgtgaatggactgaaagcacagccttggtggaccccaaaagaaacgggctacacagagttagtaaagtctttagaaagaaactggaagttaatccgagatgaaggccttgcagtgatggataaagccaaaggtctcttcctgcctgaggatgaaaacctgagggaaaaaggggactggagccagttcacgctgtggcagcaaggaagaagaaatgaaaatgcctgcaaaggagctcctaaaacctgtaccttactagaaaagttccccgagacaacaggatgcagaagaggacagatcaaatattccatcatgcaccccgggactcacgtgtggccgcacacagggcccacaaactgcaggctccgaatgcacctgggcttggtgattcccaaggaaggctgcaagattcgatgtgccaacgagaccaagacctgggaggaaggcaaggtgctcatctttgatgactcctttgagcacgaggtatggcaggatgcctcatctttccggctgatattcatcgtggatgtgtggcatccggaactgacaccacagcagagacgcagccttccagcaatttagcatgaattcatgcaagcttgggaaactctggagagaggctgcctttctggttccatctccttgggtgtgaggatagaatttcgaacaccaagagtcaattcccttgacttgcagcccgagtaattcaaagcctcctcctagggtcagaagacactaaagggaatatttgcctcgctgcaattcatttaggaaacaccctgctgtgtgtcatctcatgacagcactggtcttctgccagtatttaaggtgaacatttgatagcttctaccttaccagccaaagatattttttccacatagaataggtctaattcaatgtataatgagaacatatgtagaaactgtgaatggattgctttagtttgtaatttttctatgcagttatatttttctagtgtagctagactattttgtcatcatgtaccactacatttttgtttattttaatgacaagctgtataaatgctttacttctagctatttaatggtagcattactggggaactcagacttccctcttttaattcttcttagtaaaagatactcatgaaaaaagcagttttattttcctaacaaaaaagaaagagctcattatgtcagtgtctatgaactgtacccatcccaactctcaaatcgtttggttttttttatcttgattgagatcctcttctcactatgctagtggtggagatattgacaaaatcctatttctttcaaagaggaacttttcacaccgaaaaaagagcatggaattattttatattgttataaaaatcccagatgcaaatttttttaatgccaattattagagcttctggggaaaaagtatagttcacggaaataaaactatgttctttcagggttgggtggataggtggctgctagggtgtctggctcctggcggctttgccatccatgaggcaagggctgggaacacagtgtctttgcctatggtagatccatgtgaatgtcaggaagccagctcttcagtcttggagatgatttctgctacaattctgtagaaagattaaggatggcagagtaaaaggttaccaagaatgccaggatgtttttcttgggcgtaggaggtccagattactttcctttttgatgaaagagtttggaagactgtcccatctctctggcttgagaaatctctgccattttaaacatcactgtgaaatagcaattattatcatctgtatttagttttaacattacccacaacatagaaataataggtaaaaatcgtcttgcctactcattccaaagatgatcaagtcattaatctagcaaagtattcatgtatcagattttgtatattttgaatcaaagctaactaggaatgttagatataagaatgtaatgatattcatgcactgaattctaagccaatatgaacaaaaatgctgcatgaatggcacatataggtcaccaaagttcattcacaggtagaaaaaacttgtgctttcttttccatctaaaaacaaaaggagactttctttatctcatttaaagaacagctctttgaaattgaaattgaccctttttgcttgaccttaaggagattagcttccagtagatgagtttgcaaaatacttttcctgttcttttgttttgctggtattgaaaacatcccactaaatcagatgaagaggcatgggaggaaaaatatccaaattaattactaaaatcgagaagagaaggcaaactcttgaaaagtaaaaaggtgtttgtgaccttcagtatttattgaacagaggaaataactgacaagggcaatacaattcaatgttcatgtagtaacattcatgtcacttgttgaatttggttctcatatgtatattgcatacacataaattcaaactataagtcgtcatttttgagccatcatcttacattcatgtaatgaaattatggaagagagtaaaaactagctcttaacttagtaaatataatatggtatttaaaatcaggtcactacagtaaggttctaagtattgccaattgaaaagctagaaatggtattactgttgcaaagtgttgtcaataattgactccaatagcattgtaaatacttgtatcccacaactattttaaacccaagcaataaaatggattttctaattcacttcaattctttatttctcttacctatctatgtcttggtacataagaaaagtgtttccatcactgcattggtagaattatttcagtgttattatttttgtgatttcgtatgtctacacaaaaatgaattagtttaatttatattgtgatacagttgtttgagaaatatttttaattctgtttcaactttatttctccaagtgtataatataaaaattacttctgttattgttctctaccaataggggaaaaaattaaaacattagatccattgagaaagagatgatgtaataaataattaagattagtaataatattatcaggggtgattatgaccagttgaataatctctttcccttgaattatttagctaacaaattaactctcccaaatatttaaaataatgtaaaatcatattttactgcccattattaactaaaatatttttgtttgactttgagcaccaactggtaatactaataaatacccatgtcatgcagatggctgggcgaataagagatgtctaaaaatatgcactggtcttggaaaacatggcacaagtaaggatatcatatatgatgtctgtttattttatgtctgatttcttttgaatgagtagttggggactccatttctaaggagactaggtaaataaaatgacctttgacatttca

SEQ ID NO: 352 - DNA, 1936 bp, Homo sapiens
agtctcaagctctggtaggcaagtgcatgcagtgtgcctaaaacctgccagcagtacttttgagtttttttttttgttttgttttactttagcatttattattcatggattgaagaaatcaaaatggctgaagataaagagacaaagcatggaggacacaagaatgggaggaaaggcggactctcaggaacttcattcttcacgtggtttatggtgattgcattgctgggcgtctggacatctgtagctgtcgtttggtttgatcttgttgactatgaggaagttctaggaaaactaggaatctatgatgctgatggtgatggagattttgatgtggatgatgccaaagttttattaggacttaaagagagatctacttcagagccagcagtcccgccagaagaggctgagccacacactgagcccgaggagcaggttcctgtggaggcagaaccccagaatatcgaagatgaagcaaaagaacaaattcagtcccttctccatgaaatggtacacgcagaacatgttgagggagaagacttgcaacaagaagatggacccacaggagaaccacaacaagaggatgatgagtttcttatggcgactgatgtagatgatagatttgagaccctggaacctgaagtatctcatgaagaaaccgagcatagttaccacgtggaagagacagtttcacaagactgtaatcaggatatggaagagatgatgtctgagcaggaaaatccagattccagtgaaccagtagtagaagatgaaagattgcaccatgatacagatgatgtaacataccaagtctatgaggaacaagcagtatatgaacctctagaaaatgaagggatagaaatcacagaagtaactgctccccctgaggataatcctgtagaagattcacaggtaattgtagaagaagtaagcatttttcctgtggaagaacagcaggaagtaccaccagaaacaaatagaaaaacagatgatccagaacaaaaagcaaaagttaagaaaaagaagcctaaacttttaaataaatttgataagactattaaagctgaacttgatgctgcagaaaaactccgtaaaaggggaaaaattgaggaagcagtgaatgcatttaaagaactagtacgcaaataccctcagagtccacgagcaagatatgggaaggcgcagtgtgaggatgatttggctgagaagaggagaagtaatgaggtgctacgtggagccatcgagacctaccaagaggtggccagcctacctgatgtccctgcagacctgctgaagctgagtttgaagcgtcgctcagacaggcaacaatttctaggtcatatgagaggttccctgcttaccctgcagagattagttcaactatttcccaatgatacttccttaaaaaatgaccttggcgtgggatacctcttgataggagataatgacaatgcaaagaaagtttatgaagaggtgctgagtgtgacacctaatgatggctttgctaaagtccattatggcttcatcctgaaggcacagaacaaaattgctgagagcatcccatatttaaaggaaggaatagaatccggagatcctggcactgatgatgggagattttatttccacctgggggatgccatgcagagggttgggaacaaagaggcatataagtggtatgagcttgggcacaagagaggacactttgcatctgtctggcaacgctcactctacaatgtgaatggactgaaagcacagccttggtggaccccaaaagaaacgggctacacagagttagtaaagtctttagaaagaaactggaagttaatccgagatgaaggccttgcagtgatggataaagccaaaggtctcttcctgcctgaggatgaaaacctgagggaaaaaggggactggagccagttcacgctgtggcagcaag

SEQ ID NO: 353 - DNA, 150 bp, Homo sapiens
cctcctgtactccaggctccacctgcatcgaccgtgtggcctccttctcttgcatgtgcccagaggggaaggcagctacatctacatccaccactgggacaagccatcttgtaaaatgtgcggagaaggagaaaactttctgtgtgaatg

SEQ ID NO: 354 - DNA, 329 bp, Homo sapiens
aggctgcttcgttgcacacccgagaaagtttcagccaaacttcgggcggcggctgaggcggcggccgaggagcggcggactcggggcgcggggagtcgaggcatttgcgcctgggcttcggagcgtagcgccagggcctgagcctttgaagcaggaggaggggaggagagagtggggctcctctatcgggaccccctccccatgtggatctgcccaggcggcggcggcggcggcggaggaggaggcgaccgagaagatgcccgccctgcgccccgctctgctgtgggcgctgctggcgctctggctgtgctgcgcggcccccgcgcatg

SEQ ID NO: 355 - DNA, 82 bp, Homo sapiens
cattgcagtgtcgagatggctatgaaccctgtgtaaatgaaggaatgtgtgttacctaccacaatggcacaggatactgcaa

SEQ ID NO: 356 - DNA, 260 bp, Homo sapiens
atgtccagaaggcttcttgggggaatattgtcaacatcgagacccctgtgagaagaaccgctgccagaatggtgggacttgtgtggcccaggccatgctggggaaagccacgtgccgatgtgcctcagggtttacaggagaggactgccagtactcaacatctcatccatgctttgtgtctcgaccctgcctgaatggcggcacatgccatatgctcagccgggatacctatgagtgcacctgtcaagtcgggtttacag

SEQ ID NO: 357 - DNA, 336 bp, Homo sapiens
gtaaggagtgccaatggacggatgcctgcctgtctcatccctgtgcaaatggaagtacctgtaccactgtggccaaccagttctcctgcaaatgcctcacaggcttcacagggcagaaatgtgagactgatgtcaatgagtgtgacattccaggacactgccagcatggtggcacctgcctcaacctgcctggttcctaccagtgccagtgccctcagggcttcacaggccagtactgtgacagcctgtatgtgccctgtgcaccctcaccttgtgtcaatggaggcacctgtcggcagactggtgacttcacttttgagtgcaactgccttccag

SEQ ID NO: 358 - DNA, 123 bp, Homo sapiens
gttttgaagggagcacctgtgagaggaatattgatgactgccctaaccacaggtgtcagaatggaggggtttgtgtggatggggtcaacacttacaactgccgctgtcccccacaatggacag

SEQ ID NO: 359 - DNA, 234 bp, Homo sapiens
gacagttctgcacagaggatgtggatgaatgcctgctgcagcccaatgcctgtcaaaatgggggcacctgtgccaaccgcaatggaggctatggctgtgtatgtgtcaacggctggagtggagatgactgcagtgagaacattgatgattgtgccttcgcctcctgtactccaggctccacctgcatcgaccgtgtggcctccttctcttgcatgtgcccagaggggaaggcag

SEQ ID NO: 360 - DNA, 156 bp, Homo sapiens
gtctcctgtgtcatctggatgatgcatgcatcagcaatccttgccacaagggggcactgtgtgacaccaaccccctaaatgggcaatatatttgcacctgcccacaaggctacaaaggggctgactgcacagaagatgtggatgaatgtgccatgg

SEQ ID NO: 361 - DNA, 9905 bp, Homo sapiens
ccaatagcaatccttgtgagcatgcaggaaaatgtgtgaacacggatggcgccttccactgtgagtgtctgaagggttatgcaggacctcgttgtgagatggacatcaatgagtgccattcagacccctgccagaatgatgctacctgtctggataagattggaggcttcacatgtctgtgcatgccaggtttcaaaggtgtgcattgtgaattagaaataaatgaatgtcagagcaacccttgtgtgaacaatgggcagtgtgtggataaagtcaatcgtttccagtgcctgtgtcctcctggtttcactgggccagtttgccagattgatattgatgactgttccagtactccgtgtctgaatggggcaaagtgtatcgatcacccgaatggctatgaatgccagtgtgccacaggtttcactggtgtgttgtgtgaggagaacattgacaactgtgaccccgatccttgccaccatggtcagtgtcaggatggtattgattcctacacctgcatctgcaatcccgggtacatgggcgccatctgcagtgaccagattgatgaatgttacagcagcccttgcctgaacgatggtcgctgcattgacctggtcaatggctaccagtgcaactgccagccaggcacgtcaggggttaattgtgaaattaattttgatgactgtgcaagtaacccttgtatccatggaatctgtatggatggcattaatcgctacagttgtgtctgctcaccaggattcacagggcagagatgtaacattgacattgatgagtgtgcctccaatccctgtcgcaagggtgcaacatgtatcaacggtgtgaatggtttccgctgtatatgccccgagggaccccatcaccccagctgctactcacaggtgaacgaatgcctgagcaatccctgcatccatggaaactgtactggaggtctcagtggatataagtgtctctgtgatgcaggctgggttggcatcaactgtgaagtggacaaaaatgaatgcctttcgaatccatgccagaatggaggaacttgtgacaatctggtgaatggatacaggtgtacttgcaagaagggctttaaaggctataactgccaggtgaatattgatgaatgtgcctcaaatccatgcctgaaccaaggaacctgctttgatgacataagtggctacacttgccactgtgtgctgccatacacaggcaagaattgtcagacagtattggctccctgttccccaaacccttgtgagaatgctgctgtttgcaaagagtcaccaaattttgagagttatacttgcttgtgtgctcctggctggcaaggtcagcggtgtaccattgacattgacgagtgtatctccaagccctgcatgaaccatggtctctgccataacacccagggcagctacatgtgtgaatgtccaccaggcttcagtggtatggactgtgaggaggacattgatgactgccttgccaatccttgccagaatggaggttcctgtatggatggagtgaatactttctcctgcctctgccttccgggtttcactggggataagtgccagacagacatgaatgagtgtctgagtgaaccctgtaagaatggagggacctgctctgactacgtcaacagttacacttgcaagtgccaggcaggatttgatggagtccattgtgagaacaacatcaatgagtgcactgagagctcctgtttcaatggtggcacatgtgttgatgggattaactccttctcttgcttgtgccctgtgggtttcactggatccttctgcctccatgagatcaatgaatgcagctctcatccatgcctgaatgagggaacgtgtgttgatggcctgggtacctaccgctgcagctgccccctgggctacactgggaaaaactgtcagaccctggtgaatctctgcagtcggtctccatgtaaaaacaaaggtacttgcgttcagaaaaaagcagagtcccagtgcctatgtccatctggatgggctggtgcctattgtgacgtgcccaatgtctcttgtgacatagcagcctccaggagaggtgtgcttgttgaacacttgtgccagcactcaggtgtctgcatcaatgctggcaacacgcattactgtcagtgccccctgggctatactgggagctactgtgaggagcaactcgatgagtgtgcgtccaacccctgccagcacggggcaacatgcagtgacttcattggtggatacagatgcgagtgtgtcccaggctatcagggtgtcaactgtgagtatgaagtggatgagtgccagaatcagccctgccagaatggaggcacctgtattgaccttgtgaaccatttcaagtgctcttgcccaccaggcactcggggcctactctgtgaagagaacattgatgactgtgcccggggtccccattgccttaatggtggtcagtgcatggataggattggaggctacagttgtcgctgcttgcctggctttgctggggagcgttgtgagggagacatcaacgagtgcctctccaacccctgcagctctgagggcagcctggactgtatacagctcaccaatgactacctgtgtgtttgccgtagtgcctttactggccggcactgtgaaaccttcgtcgatgtgtgtccccagatgccctgcctgaatggagggacttgtgctgtggccagtaacatgcctgatggtttcatttgccgttgtcccccgggattttccggggcaaggtgccagagcagctgtggacaagtgaaatgtaggaagggggagcagtgtgtgcacaccgcctctggaccccgctgcttctgccccagtccccgggactgcgagtcaggctgtgccagtagcccctgccagcacgggggcagctgccaccctcagcgccagcctccttattactcctgccagtgtgccccaccattctcgggtagccgctgtgaactctacacggcaccccccagcacccctcctgccacctgtctgagccagtattgtgccgacaaagctcgggatggcgtctgtgatgaggcctgcaacagccatgcctgccagtgggatgggggtgactgttctctcaccatggagaacccctgggccaactgctcctccccacttccctgctgggattatatcaacaaccagtgtgatgagctgtgcaacacggtcgagtgcctgtttgacaactttgaatgccaggggaacagcaagacatgcaagtatgacaaatactgtgcagaccacttcaaagacaaccactgtgaccaggggtgcaacagtgaggagtgtggttgggatgggctggactgtgctgctgaccaacctgagaacctggcagaaggtaccctggttattgtggtattgatgccacctgaacaactgctccaggatgctcgcagcttcttgcgggcactgggtaccctgctccacaccaacctgcgcattaagcgggactcccagggggaactcatggtgtacccctattatggtgagaagtcagctgctatgaagaaacagaggatgacacgcagatcccttcctggtgaacaagaacaggaggtggctggctctaaagtctttctggaaattgacaaccgccagtgtgttcaagactcagaccactgcttcaagaacacggatgcagcagcagctctcctggcctctcacgccatacaggggaccctgtcataccctcttgtgtctgtcgtcagtgaatccctgactccagaacgcactcagctcctctatctccttgctgttgctgttgtcatcattctgtttattattctgctgggggtaatcatggcaaaacgaaagcgtaagcatggctctctctggctgcctgaaggtttcactcttcgccgagatgcaagcaatcacaagcgtcgtgagccagtgggacaggatgctgtggggctgaaaaatctctcagtgcaagtctcagaagctaacctaattggtactggaacaagtgaacactgggtcgatgatgaagggccccagccaaagaaagtaaaggctgaagatgaggccttactctcagaagaagatgaccccattgatcgacggccatggacacagcagcaccttgaagctgcagacatccgtaggacaccatcgctggctctcacccctcctcaggcagagcaggaggtggatgtgttagatgtgaatgtccgtggcccagatggctgcaccccattgatgttggcttctctccgaggaggcagctcagatttgagtgatgaagatgaagatgcagaggactcttctgctaacatcatcacagacttggtctaccagggtgccagcctccaggcccagacagaccggactggtgagatggccctgcaccttgcagcccgctactcacgggctgatgctgccaagcgtctcctggatgcaggtgcagatgccaatgcccaggacaacatgggccgctgtccactccatgctgcagtggcagctgatgcccaaggtgtcttccagattctgattcgcaaccgagtaactgatctagatgccaggatgaatgatggtactacacccctgatcctggctgcccgcctggctgtggagggaatggtggcagaactgatcaactgccaagcggatgtgaatgcagtggatgaccatggaaaatctgctcttcactgggcagctgctgtcaataatgtggaggcaactcttttgttgttgaaaaatggggccaaccgagacatgcaggacaacaaggaagagacacctctgtttcttgctgcccgggaggggagctatgaagcagccaagatcctgttagaccattttgccaatcgagacatcacagaccatatggatcgtcttccccgggatgtggctcgggatcgcatgcaccatgacattgtgcgccttctggatgaatacaatgtgaccccaagccctccaggcaccgtgttgacttctgctctctcacctgtcatctgtgggcccaacagatctttcctcagcctgaagcacaccccaatgggcaagaagtctagacggcccagtgccaagagtaccatgcctactagcctccctaaccttgccaaggaggcaaaggatgccaagggtagtaggaggaagaagtctctgagtgagaaggtccaactgtctgagagttcagtaactttatcccctgttgattccctagaatctcctcacacgtatgtttccgacaccacatcctctccaatgattacatcccctgggatcttacaggcctcacccaaccctatgttggccactgccgcccctcctgccccagtccatgcccagcatgcactatctttttctaaccttcatgaaatgcagcctttggcacatggggccagcactgtgcttccctcagtgagccagttgctatcccaccaccacattgtgtctccaggcagtggcagtgctggaagcttgagtaggctccatccagtcccagtcccagcagattggatgaaccgcatggaggtgaatgagacccagtacaatgagatgtttggtatggtcctggctccagctgagggcacccatcctggcatagctccccagagcaggccacctgaagggaagcacataaccacccctcgggagcccttgccccccattgtgactttccagctcatccctaaaggcagtattgcccaaccagcgggggctccccagcctcagtccacctgccctccagctgttgcgggccccctgcccaccatgtaccagattccagaaatggcccgtttgcccagtgtggctttccccactgccatgatgccccagcaggacgggcaggtagctcagaccattctcccagcctatcatcctttcccagcctctgtgggcaagtaccccacacccccttcacagcacagttatgcttcctcaaatgctgctgagcgaacacccagtcacagtggtcacctccagggtgagcatccctacctgacaccatccccagagtctcctgaccagtggtcaagttcatcaccccactctgcttctgactggtcagatgtgaccaccagccctacccctgggggtgctggaggaggtcagcggggacctgggacacacatgtctgagccaccacacaacaacatgcaggtttatgcgtgagagagtccacctccagtgtagagacataactgacttttgtaaatgctgctgaggaacaaatgaaggtcatccgggagagaaatgaagaaatctctggagccagcttctagaggtaggaaagagaagatgttcttattcagataatgcaagagaagcaattcgtcagtttcactgggtatctgcaaggcttattgattattctaatctaataagacaagtttgtggaaatgcaagatgaatacaagccttgggtccatgtttactctcttctatttggagaataagatggatgcttattgaagcccagacattcttgcagcttggactgcattttaagccctgcaggcttctgccatatccatgagaagattctacactagcgtcctgttgggaattatgccctggaattctgcctgaattgacctacgcatctcctcctccttggacattcttttgtcttcatttggtgcttttggttttgcacctctccgtgattgtagccctaccagcatgttatagggcaagacctttgtgcttttgatcattctggcccatgaaagcaactttggtctcctttcccctcctgtcttcccggtatcccttggagtctcacaaggtttactttggtatggttctcagcacaaacctttcaagtatgttgtttctttggaaaatggacatactgtattgtgttctcctgcatatatcattcctggagagagaaggggagaagaatacttttcttcaacaaattttgggggcaggagatcccttcaagaggctgcaccttaatttttcttgtctgtgtgcaggtcttcatataaactttaccaggaagaagggtgtgagtttgttgtttttctgtgtatgggcctggtcagtgtaaagttttatccttgatagtctagttactatgaccctccccacttttttaaaaccagaaaaaggtttggaatgttggaatgaccaagagacaagttaactcgtgcaagagccagttacccacccacaggtccccctacttcctgccaagcattccattgactgcctgtatggaacacatttgtcccagatctgagcattctaggcctgtttcactcactcacccagcatatgaaactagtcttaactgttgagcctttcctttcatatccacagaagacactgtctcaaatgttgtacccttgccatttaggactgaactttccttagcccaagggacccagtgacagttgtcttccgtttgtcagatgatcagtctctactgattatcttgctgcttaaaggcctgctcaccaatctttctttcacaccgtgtggtccgtgttactggtatacccagtatgttctcactgaagacatggactttatatgttcaagtgcaggaattggaaagttggacttgttttctatgatccaaaacagccctataagaaggttggaaaaggaggaactatatagcagcctttgctattttctgctaccatttcttttcctctgaagcggccatgacattccctttggcaactaacgtagaaactcaacagaacattttcctttcctagagtcaccttttagatgataatggacaactatagacttgctcattgttcagactgattgcccctcacctgaatccactctctgtattcatgctcttggcaatttctttgactttcttttaagggcagaagcattttagttaattgtagataaagaatagttttcttcctcttctccttgggccagttaataattggtccatggctacactgcaacttccgtccagtgctgtgatgcccatgacacctgcaaaataagttctgcctgggcattttgtagatattaacaggtgaattcccgactcttttggtttgaatgacagttctcattccttctatggctgcaagtatgcatcagtgcttcccacttacctgatttgtctgtcggtggccccatatggaaaccctgcgtgtctgttggcataatagtttacaaatggttttttcagtcctatccaaatttattgaaccaacaaaaataattacttctgccctgagataagcagattaagtttgttcattctctgctttattctctccatgtggcaacattctgtcagcctctttcatagtgtgcaaacattttatcattctaaatggtgactctctgcccttggacccatttattattcacagatggggagaacctatctgcatggacctctgtggaccacagcgtacctgcccctttctgccctcctgctccagccccacttctgaaagtatcagctactgatccagccactggatattttatatcctcccttttccttaagcacaatgtcagaccaaattgcttgtttctttttcttggactactttaatttggatcctttgggtttggagaaagggaatgtgaaagctgtcattacagacaacaggtttcagtgatgaggaggacaacactgcctttcaaactttttactgatctcttagattttaagaactcttgaattgtgtggtatctaataaaagggaaggtaagatggataatcactttctcatttgggttctgaattggagactcagtttttatgagacacatcttttatgccatgtatagatcctcccctgctatttttggtttatttttattgttataaatgctttctttctttgactcctcttctgcctgcctttggggataggtttttttgtttgtttatttgcttcctctgttttgttttaagcatcattttcttatgtgaggtggggaagggaaaggtatgagggaaagagagtctgagaattaaaatattttagtataagcaattggctgtgatgctcaaatccattgcatcctcttattgaatttgccaatttgtaatttttgcataataaagaaccaaaggtgtaatgttttgttgagaggtggtttagggattttggccctaaccaatacattgaatgtatgatgactatttgggaggacacatttatgtacccagaggcccccactaataagtggtactatggttacttccttgtgtacatttctcttaaaagtgatattatatctgtttgtatgagaaacccagtaaccaataaaatgaccgcatattcctgactaaacgtagtaaggaaaatgcacactttgtttttacttttccgtttcattctaaaggtagttaagatgaaatttatatgaaagcatttttatcacaaaataaaaaaggtttgccaagctcagtggtgttgtattttttattttccaatactgcatccatggcctggcagtgttacctcatgatgtcataatttgctgagagagcaaattttcttttctttctgaatcccacaaagcctagcaccaaacttctttttttcttcctttaattagatcataaataaatgatcctggggaaaaagcatctgtcaaataggaaacatcacaaaactgagcactcttctgtgcactagccatagctggtgacaaacagatggttgctcagggacaaggtgccttccaatggaaatgcgaagtagttgctatagcaagaattgggaactgggatataagtcataatattaattatgctgttatgtaaatgattggtttgtaacattccttaagtgaaatttgtgtagaacttaatatacaggattataaaataatattttgtgtataaatttgttataagttcacattcatacatttatttataaagtcagtgagatatttgaacatgaa

SEQ ID NO: 362 - DNA, 11425 bp, Homo sapiens
aggctgcttcgttgcacacccgagaaagtttcagccaaacttcgggcggcggctgaggcggcggccgaggagcggcggactcggggcgcggggagtcgaggcatttgcgcctgggcttcggagcgtagcgccagggcctgagcctttgaagcaggaggaggggaggagagagtggggctcctctatcgggaccccctccccatgtggatctgcccaggcggcggcggcggcggcggaggaggaggcgaccgagaagatgcccgccctgcgccccgctctgctgtgggcgctgctggcgctctggctgtgctgcgcggcccccgcgcatgcattgcagtgtcgagatggctatgaaccctgtgtaaatgaaggaatgtgtgttacctaccacaatggcacaggatactgcaaatgtccagaaggcttcttgggggaatattgtcaacatcgagacccctgtgagaagaaccgctgccagaatggtgggacttgtgtggcccaggccatgctggggaaagccacgtgccgatgtgcctcagggtttacaggagaggactgccagtactcaacatctcatccatgctttgtgtctcgaccctgcctgaatggcggcacatgccatatgctcagccgggatacctatgagtgcacctgtcaagtcgggtttacaggtaaggagtgccaatggacggatgcctgcctgtctcatccctgtgcaaatggaagtacctgtaccactgtggccaaccagttctcctgcaaatgcctcacaggcttcacagggcagaaatgtgagactgatgtcaatgagtgtgacattccaggacactgccagcatggtggcacctgcctcaacctgcctggttcctaccagtgccagtgccctcagggcttcacaggccagtactgtgacagcctgtatgtgccctgtgcaccctcaccttgtgtcaatggaggcacctgtcggcagactggtgacttcacttttgagtgcaactgccttccaggttttgaagggagcacctgtgagaggaatattgatgactgccctaaccacaggtgtcagaatggaggggtttgtgtggatggggtcaacacttacaactgccgctgtcccccacaatggacaggacagttctgcacagaggatgtggatgaatgcctgctgcagcccaatgcctgtcaaaatgggggcacctgtgccaaccgcaatggaggctatggctgtgtatgtgtcaacggctggagtggagatgactgcagtgagaacattgatgattgtgccttcgcctcctgtactccaggctccacctgcatcgaccgtgtggcctccttctcttgcatgtgcccagaggggaaggcaggtctcctgtgtcatctggatgatgcatgcatcagcaatccttgccacaagggggcactgtgtgacaccaaccccctaaatgggcaatatatttgcacctgcccacaaggctacaaaggggctgactgcacagaagatgtggatgaatgtgccatggccaatagcaatccttgtgagcatgcaggaaaatgtgtgaacacggatggcgccttccactgtgagtgtctgaagggttatgcaggacctcgttgtgagatggacatcaatgagtgccattcagacccctgccagaatgatgctacctgtctggataagattggaggcttcacatgtctgtgcatgccaggtttcaaaggtgtgcattgtgaattagaaataaatgaatgtcagagcaacccttgtgtgaacaatgggcagtgtgtggataaagtcaatcgtttccagtgcctgtgtcctcctggtttcactgggccagtttgccagattgatattgatgactgttccagtactccgtgtctgaatggggcaaagtgtatcgatcacccgaatggctatgaatgccagtgtgccacaggtttcactggtgtgttgtgtgaggagaacattgacaactgtgaccccgatccttgccaccatggtcagtgtcaggatggtattgattcctacacctgcatctgcaatcccgggtacatgggcgccatctgcagtgaccagattgatgaatgttacagcagcccttgcctgaacgatggtcgctgcattgacctggtcaatggctaccagtgcaactgccagccaggcacgtcaggggttaattgtgaaattaattttgatgactgtgcaagtaacccttgtatccatggaatctgtatggatggcattaatcgctacagttgtgtctgctcaccaggattcacagggcagagatgtaacattgacattgatgagtgtgcctccaatccctgtcgcaagggtgcaacatgtatcaacggtgtgaatggtttccgctgtatatgccccgagggaccccatcaccccagctgctactcacaggtgaacgaatgcctgagcaatccctgcatccatggaaactgtactggaggtctcagtggatataagtgtctctgtgatgcaggctgggttggcatcaactgtgaagtggacaaaaatgaatgcctttcgaatccatgccagaatggaggaacttgtgacaatctggtgaatggatacaggtgtacttgcaagaagggctttaaaggctataactgccaggtgaatattgatgaatgtgcctcaaatccatgcctgaaccaaggaacctgctttgatgacataagtggctacacttgccactgtgtgctgccatacacaggcaagaattgtcagacagtattggctccctgttccccaaacccttgtgagaatgctgctgtttgcaaagagtcaccaaattttgagagttatacttgcttgtgtgctcctggctggcaaggtcagcggtgtaccattgacattgacgagtgtatctccaagccctgcatgaaccatggtctctgccataacacccagggcagctacatgtgtgaatgtccaccaggcttcagtggtatggactgtgaggaggacattgatgactgccttgccaatccttgccagaatggaggttcctgtatggatggagtgaatactttctcctgcctctgccttccgggtttcactggggataagtgccagacagacatgaatgagtgtctgagtgaaccctgtaagaatggagggacctgctctgactacgtcaacagttacacttgcaagtgccaggcaggatttgatggagtccattgtgagaacaacatcaatgagtgcactgagagctcctgtttcaatggtggcacatgtgttgatgggattaactccttctcttgcttgtgccctgtgggtttcactggatccttctgcctccatgagatcaatgaatgcagctctcatccatgcctgaatgagggaacgtgtgttgatggcctgggtacctaccgctgcagctgccccctgggctacactgggaaaaactgtcagaccctggtgaatctctgcagtcggtctccatgtaaaaacaaaggtacttgcgttcagaaaaaagcagagtcccagtgcctatgtccatctggatgggctggtgcctattgtgacgtgcccaatgtctcttgtgacatagcagcctccaggagaggtgtgcttgttgaacacttgtgccagcactcaggtgtctgcatcaatgctggcaacacgcattactgtcagtgccccctgggctatactgggagctactgtgaggagcaactcgatgagtgtgcgtccaacccctgccagcacggggcaacatgcagtgacttcattggtggatacagatgcgagtgtgtcccaggctatcagggtgtcaactgtgagtatgaagtggatgagtgccagaatcagccctgccagaatggaggcacctgtattgaccttgtgaaccatttcaagtgctcttgcccaccaggcactcggggcctactctgtgaagagaacattgatgactgtgcccggggtccccattgccttaatggtggtcagtgcatggataggattggaggctacagttgtcgctgcttgcctggctttgctggggagcgttgtgagggagacatcaacgagtgcctctccaacccctgcagctctgagggcagcctggactgtatacagctcaccaatgactacctgtgtgtttgccgtagtgcctttactggccggcactgtgaaaccttcgtcgatgtgtgtccccagatgccctgcctgaatggagggacttgtgctgtggccagtaacatgcctgatggtttcatttgccgttgtcccccgggattttccggggcaaggtgccagagcagctgtggacaagtgaaatgtaggaagggggagcagtgtgtgcacaccgcctctggaccccgctgcttctgccccagtccccgggactgcgagtcaggctgtgccagtagcccctgccagcacgggggcagctgccaccctcagcgccagcctccttattactcctgccagtgtgccccaccattctcgggtagccgctgtgaactctacacggcaccccccagcacccctcctgccacctgtctgagccagtattgtgccgacaaagctcgggatggcgtctgtgatgaggcctgcaacagccatgcctgccagtgggatgggggtgactgttctctcaccatggagaacccctgggccaactgctcctccccacttccctgctgggattatatcaacaaccagtgtgatgagctgtgcaacacggtcgagtgcctgtttgacaactttgaatgccaggggaacagcaagacatgcaagtatgacaaatactgtgcagaccacttcaaagacaaccactgtgaccaggggtgcaacagtgaggagtgtggttgggatgggctggactgtgctgctgaccaacctgagaacctggcagaaggtaccctggttattgtggtattgatgccacctgaacaactgctccaggatgctcgcagcttcttgcgggcactgggtaccctgctccacaccaacctgcgcattaagcgggactcccagggggaactcatggtgtacccctattatggtgagaagtcagctgctatgaagaaacagaggatgacacgcagatcccttcctggtgaacaagaacaggaggtggctggctctaaagtctttctggaaattgacaaccgccagtgtgttcaagactcagaccactgcttcaagaacacggatgcagcagcagctctcctggcctctcacgccatacaggggaccctgtcataccctcttgtgtctgtcgtcagtgaatccctgactccagaacgcactcagctcctctatctccttgctgttgctgttgtcatcattctgtttattattctgctgggggtaatcatggcaaaacgaaagcgtaagcatggctctctctggctgcctgaaggtttcactcttcgccgagatgcaagcaatcacaagcgtcgtgagccagtgggacaggatgctgtggggctgaaaaatctctcagtgcaagtctcagaagctaacctaattggtactggaacaagtgaacactgggtcgatgatgaagggccccagccaaagaaagtaaaggctgaagatgaggccttactctcagaagaagatgaccccattgatcgacggccatggacacagcagcaccttgaagctgcagacatccgtaggacaccatcgctggctctcacccctcctcaggcagagcaggaggtggatgtgttagatgtgaatgtccgtggcccagatggctgcaccccattgatgttggcttctctccgaggaggcagctcagatttgagtgatgaagatgaagatgcagaggactcttctgctaacatcatcacagacttggtctaccagggtgccagcctccaggcccagacagaccggactggtgagatggccctgcaccttgcagcccgctactcacgggctgatgctgccaagcgtctcctggatgcaggtgcagatgccaatgcccaggacaacatgggccgctgtccactccatgctgcagtggcagctgatgcccaaggtgtcttccagattctgattcgcaaccgagtaactgatctagatgccaggatgaatgatggtactacacccctgatcctggctgcccgcctggctgtggagggaatggtggcagaactgatcaactgccaagcggatgtgaatgcagtggatgaccatggaaaatctgctcttcactgggcagctgctgtcaataatgtggaggcaactcttttgttgttgaaaaatggggccaaccgagacatgcaggacaacaaggaagagacacctctgtttcttgctgcccgggaggggagctatgaagcagccaagatcctgttagaccattttgccaatcgagacatcacagaccatatggatcgtcttccccgggatgtggctcgggatcgcatgcaccatgacattgtgcgccttctggatgaatacaatgtgaccccaagccctccaggcaccgtgttgacttctgctctctcacctgtcatctgtgggcccaacagatctttcctcagcctgaagcacaccccaatgggcaagaagtctagacggcccagtgccaagagtaccatgcctactagcctccctaaccttgccaaggaggcaaaggatgccaagggtagtaggaggaagaagtctctgagtgagaaggtccaactgtctgagagttcagtaactttatcccctgttgattccctagaatctcctcacacgtatgtttccgacaccacatcctctccaatgattacatcccctgggatcttacaggcctcacccaaccctatgttggccactgccgcccctcctgccccagtccatgcccagcatgcactatctttttctaaccttcatgaaatgcagcctttggcacatggggccagcactgtgcttccctcagtgagccagttgctatcccaccaccacattgtgtctccaggcagtggcagtgctggaagcttgagtaggctccatccagtcccagtcccagcagattggatgaaccgcatggaggtgaatgagacccagtacaatgagatgtttggtatggtcctggctccagctgagggcacccatcctggcatagctccccagagcaggccacctgaagggaagcacataaccacccctcgggagcccttgccccccattgtgactttccagctcatccctaaaggcagtattgcccaaccagcgggggctccccagcctcagtccacctgccctccagctgttgcgggccccctgcccaccatgtaccagattccagaaatggcccgtttgcccagtgtggctttccccactgccatgatgccccagcaggacgggcaggtagctcagaccattctcccagcctatcatcctttcccagcctctgtgggcaagtaccccacacccccttcacagcacagttatgcttcctcaaatgctgctgagcgaacacccagtcacagtggtcacctccagggtgagcatccctacctgacaccatccccagagtctcctgaccagtggtcaagttcatcaccccactctgcttctgactggtcagatgtgaccaccagccctacccctgggggtgctggaggaggtcagcggggacctgggacacacatgtctgagccaccacacaacaacatgcaggtttatgcgtgagagagtccacctccagtgtagagacataactgacttttgtaaatgctgctgaggaacaaatgaaggtcatccgggagagaaatgaagaaatctctggagccagcttctagaggtaggaaagagaagatgttcttattcagataatgcaagagaagcaattcgtcagtttcactgggtatctgcaaggcttattgattattctaatctaataagacaagtttgtggaaatgcaagatgaatacaagccttgggtccatgtttactctcttctatttggagaataagatggatgcttattgaagcccagacattcttgcagcttggactgcattttaagccctgcaggcttctgccatatccatgagaagattctacactagcgtcctgttgggaattatgccctggaattctgcctgaattgacctacgcatctcctcctccttggacattcttttgtcttcatttggtgcttttggttttgcacctctccgtgattgtagccctaccagcatgttatagggcaagacctttgtgcttttgatcattctggcccatgaaagcaactttggtctcctttcccctcctgtcttcccggtatcccttggagtctcacaaggtttactttggtatggttctcagcacaaacctttcaagtatgttgtttctttggaaaatggacatactgtattgtgttctcctgcatatatcattcctggagagagaaggggagaagaatacttttcttcaacaaattttgggggcaggagatcccttcaagaggctgcaccttaatttttcttgtctgtgtgcaggtcttcatataaactttaccaggaagaagggtgtgagtttgttgtttttctgtgtatgggcctggtcagtgtaaagttttatccttgatagtctagttactatgaccctccccacttttttaaaaccagaaaaaggtttggaatgttggaatgaccaagagacaagttaactcgtgcaagagccagttacccacccacaggtccccctacttcctgccaagcattccattgactgcctgtatggaacacatttgtcccagatctgagcattctaggcctgtttcactcactcacccagcatatgaaactagtcttaactgttgagcctttcctttcatatccacagaagacactgtctcaaatgttgtacccttgccatttaggactgaactttccttagcccaagggacccagtgacagttgtcttccgtttgtcagatgatcagtctctactgattatcttgctgcttaaaggcctgctcaccaatctttctttcacaccgtgtggtccgtgttactggtatacccagtatgttctcactgaagacatggactttatatgttcaagtgcaggaattggaaagttggacttgttttctatgatccaaaacagccctataagaaggttggaaaaggaggaactatatagcagcctttgctattttctgctaccatttcttttcctctgaagcggccatgacattccctttggcaactaacgtagaaactcaacagaacattttcctttcctagagtcaccttttagatgataatggacaactatagacttgctcattgttcagactgattgcccctcacctgaatccactctctgtattcatgctcttggcaatttctttgactttcttttaagggcagaagcattttagttaattgtagataaagaatagttttcttcctcttctccttgggccagttaataattggtccatggctacactgcaacttccgtccagtgctgtgatgcccatgacacctgcaaaataagttctgcctgggcattttgtagatattaacaggtgaattcccgactcttttggtttgaatgacagttctcattccttctatggctgcaagtatgcatcagtgcttcccacttacctgatttgtctgtcggtggccccatatggaaaccctgcgtgtctgttggcataatagtttacaaatggttttttcagtcctatccaaatttattgaaccaacaaaaataattacttctgccctgagataagcagattaagtttgttcattctctgctttattctctccatgtggcaacattctgtcagcctctttcatagtgtgcaaacattttatcattctaaatggtgactctctgcccttggacccatttattattcacagatggggagaacctatctgcatggacctctgtggaccacagcgtacctgcccctttctgccctcctgctccagccccacttctgaaagtatcagctactgatccagccactggatattttatatcctcccttttccttaagcacaatgtcagaccaaattgcttgtttctttttcttggactactttaatttggatcctttgggtttggagaaagggaatgtgaaagctgtcattacagacaacaggtttcagtgatgaggaggacaacactgcctttcaaactttttactgatctcttagattttaagaactcttgaattgtgtggtatctaataaaagggaaggtaagatggataatcactttctcatttgggttctgaattggagactcagtttttatgagacacatcttttatgccatgtatagatcctcccctgctatttttggtttatttttattgttataaatgctttctttctttgactcctcttctgcctgcctttggggataggtttttttgtttgtttatttgcttcctctgttttgttttaagcatcattttcttatgtgaggtggggaagggaaaggtatgagggaaagagagtctgagaattaaaatattttagtataagcaattggctgtgatgctcaaatccattgcatcctcttattgaatttgccaatttgtaatttttgcataataaagaaccaaaggtgtaatgttttgttgagaggtggtttagggattttggccctaaccaatacattgaatgtatgatgactatttgggaggacacatttatgtacccagaggcccccactaataagtggtactatggttacttccttgtgtacatttctcttaaaagtgatattatatctgtttgtatgagaaacccagtaaccaataaaatgaccgcatattcctgactaaacgtagtaaggaaaatgcacactttgtttttacttttccgtttcattctaaaggtagttaagatgaaatttatatgaaagcatttttatcacaaaataaaaaaggtttgccaagctcagtggtgttgtattttttattttccaatactgcatccatggcctggcagtgttacctcatgatgtcataatttgctgagagagcaaattttcttttctttctgaatcccacaaagcctagcaccaaacttctttttttcttcctttaattagatcataaataaatgatcctggggaaaaagcatctgtcaaataggaaacatcacaaaactgagcactcttctgtgcactagccatagctggtgacaaacagatggttgctcagggacaaggtgccttccaatggaaatgcgaagtagttgctatagcaagaattgggaactgggatataagtcataatattaattatgctgttatgtaaatgattggtttgtaacattccttaagtgaaatttgtgtagaacttaatatacaggattataaaataatattttgtgtataaatttgttataagttcacattcatacatttatttataaagtcagtgagatatttgaacatgaa

SEQ ID NO: 363 - DNA, 1364 bp, Homo sapiens
aggctgcttcgttgcacacccgagaaagtttcagccaaacttcgggcggcggctgaggcggcggccgaggagcggcggactcggggcgcggggagtcgaggcatttgcgcctgggcttcggagcgtagcgccagggcctgagcctttgaagcaggaggaggggaggagagagtggggctcctctatcgggaccccctccccatgtggatctgcccaggcggcggcggcggcggcggaggaggaggcgaccgagaagatgcccgccctgcgccccgctctgctgtgggcgctgctggcgctctggctgtgctgcgcggcccccgcgcatgcattgcagtgtcgagatggctatgaaccctgtgtaaatgaaggaatgtgtgttacctaccacaatggcacaggatactgcaaatgtccagaaggcttcttgggggaatattgtcaacatcgagacccctgtgagaagaaccgctgccagaatggtgggacttgtgtggcccaggccatgctggggaaagccacgtgccgatgtgcctcagggtttacaggagaggactgccagtactcaacatctcatccatgctttgtgtctcgaccctgcctgaatggcggcacatgccatatgctcagccgggatacctatgagtgcacctgtcaagtcgggtttacaggtaaggagtgccaatggacggatgcctgcctgtctcatccctgtgcaaatggaagtacctgtaccactgtggccaaccagttctcctgcaaatgcctcacaggcttcacagggcagaaatgtgagactgatgtcaatgagtgtgacattccaggacactgccagcatggtggcacctgcctcaacctgcctggttcctaccagtgccagtgccctcagggcttcacaggccagtactgtgacagcctgtatgtgccctgtgcaccctcaccttgtgtcaatggaggcacctgtcggcagactggtgacttcacttttgagtgcaactgccttccaggttttgaagggagcacctgtgagaggaatattgatgactgccctaaccacaggtgtcagaatggaggggtttgtgtggatggggtcaacacttacaactgccgctgtcccccacaatggacaggacagttctgcacagaggatgtggatgaatgcctgctgcagcccaatgcctgtcaaaatgggggcacctgtgccaaccgcaatggaggctatggctgtgtatgtgtcaacggctggagtggagatgactgcagtgagaacattgatgattgtgccttcgcctcctgtactccaggctccacctgcatcgaccgtgtggcctccttctcttgcatgtgcccagaggggaaggcag

SEQ ID NO: 364 - DNA, 150 bp, Homo sapiens
agggccggctggacaaactgacagtcacctcccagaacctgcagctggagaacctgcgcatgaagcttcccaagcccttgcctccccgattgaaagagatgaaaagccaggaatcggctgcaggttccaaactagtccttcggtgtgaaa

SEQ ID NO: 365 - DNA, 286 bp, Homo sapiens
atcctgccccgcaaaaggcagcttcaccaaagtggggtatttccagcctttgtagctttcacttccacatctaccaagtgggcggagtggccttctgtggacgaatcagattcctctccagcaccgactttaagaggcgagccggggggtcagggtcccagatgcacaggaggagaagcaggagctgtcgggaagatcagaagccagtcatggatgaccagcgcgaccttatctccaacaatgagcaactgcccatgctgggccggcgccctggggccccggagag

SEQ ID NO: 366 - DNA, 173 bp, Homo sapiens
caagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagc

SEQ ID NO: 367 - DNA, 80 bp, Homo sapiens
ctcccaagcctgtgagcaagatgcgcatggccaccccgctgctgatgcaggcgctgcccatgggagccctgccccagggg

SEQ ID NO: 368 - DNA, 63 bp, Homo sapiens
cccatgcagaatgccaccaagtatggcaacatgacagaggaccatgtgatgcacctgctccag

SEQ ID NO: 369 - DNA, 96 bp, Homo sapiens
aatgctgaccccctgaaggtgtacccgccactgaaggggagcttcccggagaacctgagacaccttaagaacaccatggagaccatagactggaag

SEQ ID NO: 370 - DNA, 88 bp, Homo sapiens
gtctttgagagctggatgcaccattggctcctgtttgaaatgagcaggcactccttggagcaaaagcccactgacgctccaccgaaag

SEQ ID NO: 371 - DNA, 192 bp, Homo sapiens
tactgaccaagtgccaggaagaggtcagccacatccctgctgtccacccgggttcattcaggcccaagtgcgacgagaacggcaactatctgccactccagtgctatgggagcatcggctactgctggtgtgtcttccccaacggcacggaggtccccaacaccagaagccgcgggcaccataactgcagtg

SEQ ID NO: 372 - DNA, 63 bp, Homo sapiens
agtcactggaactggaggacccgtcttctgggctgggtgtgaccaagcaggatctgggcccag

SEQ ID NO: 373 - DNA, 612 bp, Homo sapiens
tccccatgtgagagcagcagaggcggtcttcaacatcctgccagccccacacagctacagctttcttgctcccttcagcccccagcccctcccccatctcccaccctgtacctcatcccatgagaccctggtgcctggctctttcgtcacccttggacaagacaaaccaagtcggaacagcagataacaatgcagcaaggccctgctgcccaatctccatctgtcaacaggggcgtgaggtcccaggaagtggccaaaagctagacagatccccgttcctgacatcacagcagcctccaacacaaggctccaagacctaggctcatggacgagatgggaaggcacagggagaagggataaccctacacccagaccccaggctggacatgctgactgtcctctcccctccagcctttggccttggcttttctagcctatttacctgcaggctgagccactctcttccctttccccagcatcactccccaaggaagagccaatgttttccacccataatcctttctgccgacccctagttccctctgctcagccaagcttgttatcagctttcagggccatggttcacattagaataaaaggtagtaattagaa

SEQ ID NO: 374 - DNA, 1653 bp, Homo sapiens
atcctgccccgcaaaaggcagcttcaccaaagtggggtatttccagcctttgtagctttcacttccacatctaccaagtgggcggagtggccttctgtggacgaatcagattcctctccagcaccgactttaagaggcgagccggggggtcagggtcccagatgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagtactgaccaagtgccaggaagaggtcagccacatccctgctgtccacccgggttcattcaggcccaagtgcgacgagaacggcaactatctgccactccagtgctatgggagcatcggctactgctggtgtgtcttccccaacggcacggaggtccccaacaccagaagccgcgggcaccataactgcagtgagtcactggaactggaggacccgtcttctgggctgggtgtgaccaagcaggatctgggcccagtccccatgtgagagcagcagaggcggtcttcaacatcctgccagccccacacagctacagctttcttgctcccttcagcccccagcccctcccccatctcccaccctgtacctcatcccatgagaccctggtgcctggctctttcgtcacccttggacaagacaaaccaagtcggaacagcagataacaatgcagcaaggccctgctgcccaatctccatctgtcaacaggggcgtgaggtcccaggaagtggccaaaagctagacagatccccgttcctgacatcacagcagcctccaacacaaggctccaagacctaggctcatggacgagatgggaaggcacagggagaagggataaccctacacccagaccccaggctggacatgctgactgtcctctcccctccagcctttggccttggcttttctagcctatttacctgcaggctgagccactctcttccctttccccagcatcactccccaaggaagagccaatgttttccacccataatcctttctgccgacccctagttccctctgctcagccaagcttgttatcagctttcagggccatggttcacattagaataaaaggtagtaattagaa

SEQ ID NO: 375 - DNA, 459 bp, Homo sapiens
atcctgccccgcaaaaggcagcttcaccaaagtggggtatttccagcctttgtagctttcacttccacatctaccaagtgggcggagtggccttctgtggacgaatcagattcctctccagcaccgactttaagaggcgagccggggggtcagggtcccagatgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagc

SEQ ID NO: 376 - DNA, 150 bp, Homo sapiens
taccagacgatgaggatgtagtggggcccgggcaggaatctgatgactttgagctgtctggctctggagatctggccttgcctccccgattgaaagagatgaaaagccaggaatcggctgcaggttccaaactagtccttcggtgtgaaa

SEQ ID NO: 377 - DNA, 100 bp, Homo sapiens
actcgccgcagcctgcgcgccttctccagtccgcggtgccatggcccccgcccgtctgttcgcgctgctgctgttcttcgtaggcggagtcgccgagtcg

SEQ ID NO: 378 - DNA, 139 bp, Homo sapiens
atccgagagactgaggtcatcgacccccaggacctcctagaaggccgatacttctccggagccctaccagacgatgaggatgtagtggggcccgggcaggaatctgatgactttgagctgtctggctctggagatctgg

SEQ ID NO: 379 - DNA, 47 bp, Homo sapiens
atgacttggaagactccatgatcggccctgaagttgtccatcccttg

SEQ ID NO: 380 - DNA, 199 bp, Homo sapiens
gtgcctctagataaccatatccctgagagggcagggtctgggagccaagtccccaccgaacccaagaaactagaggagaatgaggttatccccaagagaatctcacccgttgaagagagtgaggatgtgtccaacaaggtgtcaatgtccagcactgtgcagggcagcaacatctttgagagaacggaggtcctggcag

SEQ ID NO: 381 - DNA, 2128 bp, Homo sapiens
ctctgattgtgggtggcatcgtgggcatcctctttgccgtcttcctgatcctactgctcatgtaccgtatgaagaagaaggatgaaggcagctatgacctgggcaagaaacccatctacaagaaagcccccaccaatgagttctacgcgtgaagcttgcttgtgggcactggcttggactttagcggggagggaagccaggggattttgaagggtggacattagggtagggtgaggtcaacctaatactgacttgtcagtatctccagctctgattacctttgaagtgttcagaagagacattgtcttctactgttctgccaggttcttcttgagctttgggcctcagttgccctggcagaaaaatggattcaacttggcctttctgaaggcaagactgggattggatcacttcttaaacttccagttaagaatctaggtccgccctcaagcccatactgaccatgcctcatccagagctcctctgaagccagggggctaacggatgttgtgtggagtcctggctggaggtcctcccccagtggccttcctcccttcctttcacagccggtctctctgccaggaaatgggggaaggaactagaaccacctgcaccttgagatgtttctgtaaatgggtacttgtgatcacactacgggaatctctgtggtatatacctggggccattctaggctctttcaagtgacttttggaaatcaaccttttttatttgggggggaggatggggaaaagagctgagagtttatgctgaaatggatttatagaatatttgtaaatctatttttagtgtttgttcgtttttttaactgttcattcctttgtgcagagtgtatatctctgcctgggcaagagtgtggaggtgccgaggtgtcttcattctctcgcacatttccacagcacctgctaagtttgtatttaatggtttttgtttttgtttttgtttgtttcttgaaaatgagagaagagccggagagatgatttttattaattttttttttttttttttttttttactatttatagctttagatagggcctcccttcccctcttctttctttgttctctttcattaaaccccttccccagttttttttttatactttaaaccccgctcctcatggccttggccctttctgaagctgcttcctcttataaaatagcttttgccgaaacatagtttttttttagcagatcccaaaatataatgaaggggatggtgggatatttgtgtctgtgttcttataatatattattattcttccttggttctagaaaaatagataaatatatttttttcaggaaatagtgtggtgtttccagtttgatgttgctgggtggttgagtgagtgaattttcatgtggctgggtgggtttttgcctttttctcttgccctgttcctggtgccttctgatggggctggaatagttgaggtggatggttctaccctttctgccttctgtttgggacccagctggtgttctttggtttgctttcttcaggctctagggctgtgctatccaatacagtaaccacatgcggctgtttaaagttaagccaattaaaatcacataagattaaaaattccttcctcagttgcactaaccacgtttctagaggcgtcactgtatgtagttcatggctactgtactgacagcgagagcatgtccatctgttggacagcactattctagagaactaaactggcttaacgagtcacagcctcagctgtgctgggacgacccttgtctccctgggtaggggggggggaatgggggagggctgatgaggccccagctggggcctgttgtctgggaccctccctctcctgagaggggaggcctggtggcttagcctgggcaggtcgtgtctcctcctgaccccagtggctgcggtgaggggaaccaccctcccttgctgcaccagtggccattagctcccgtcaccactgcaacccagggtcccagctggctgggtcctcttctgcccccagtgcccttccccttgggctgtgttggagtgagcacctcctctgtaggcacctctcacactgttgtctgttactgattttttttgataaaaagataataaaacctggtactttctaaa

SEQ ID NO: 382 - DNA, 2613 bp, Homo sapiens
actcgccgcagcctgcgcgccttctccagtccgcggtgccatggcccccgcccgtctgttcgcgctgctgctgttcttcgtaggcggagtcgccgagtcgatccgagagactgaggtcatcgacccccaggacctcctagaaggccgatacttctccggagccctaccagacgatgaggatgtagtggggcccgggcaggaatctgatgactttgagctgtctggctctggagatctggatgacttggaagactccatgatcggccctgaagttgtccatcccttggtgcctctagataaccatatccctgagagggcagggtctgggagccaagtccccaccgaacccaagaaactagaggagaatgaggttatccccaagagaatctcacccgttgaagagagtgaggatgtgtccaacaaggtgtcaatgtccagcactgtgcagggcagcaacatctttgagagaacggaggtcctggcagctctgattgtgggtggcatcgtgggcatcctctttgccgtcttcctgatcctactgctcatgtaccgtatgaagaagaaggatgaaggcagctatgacctgggcaagaaacccatctacaagaaagcccccaccaatgagttctacgcgtgaagcttgcttgtgggcactggcttggactttagcggggagggaagccaggggattttgaagggtggacattagggtagggtgaggtcaacctaatactgacttgtcagtatctccagctctgattacctttgaagtgttcagaagagacattgtcttctactgttctgccaggttcttcttgagctttgggcctcagttgccctggcagaaaaatggattcaacttggcctttctgaaggcaagactgggattggatcacttcttaaacttccagttaagaatctaggtccgccctcaagcccatactgaccatgcctcatccagagctcctctgaagccagggggctaacggatgttgtgtggagtcctggctggaggtcctcccccagtggccttcctcccttcctttcacagccggtctctctgccaggaaatgggggaaggaactagaaccacctgcaccttgagatgtttctgtaaatgggtacttgtgatcacactacgggaatctctgtggtatatacctggggccattctaggctctttcaagtgacttttggaaatcaaccttttttatttgggggggaggatggggaaaagagctgagagtttatgctgaaatggatttatagaatatttgtaaatctatttttagtgtttgttcgtttttttaactgttcattcctttgtgcagagtgtatatctctgcctgggcaagagtgtggaggtgccgaggtgtcttcattctctcgcacatttccacagcacctgctaagtttgtatttaatggtttttgtttttgtttttgtttgtttcttgaaaatgagagaagagccggagagatgatttttattaattttttttttttttttttttttttactatttatagctttagatagggcctcccttcccctcttctttctttgttctctttcattaaaccccttccccagttttttttttatactttaaaccccgctcctcatggccttggccctttctgaagctgcttcctcttataaaatagcttttgccgaaacatagtttttttttagcagatcccaaaatataatgaaggggatggtgggatatttgtgtctgtgttcttataatatattattattcttccttggttctagaaaaatagataaatatatttttttcaggaaatagtgtggtgtttccagtttgatgttgctgggtggttgagtgagtgaattttcatgtggctgggtgggtttttgcctttttctcttgccctgttcctggtgccttctgatggggctggaatagttgaggtggatggttctaccctttctgccttctgtttgggacccagctggtgttctttggtttgctttcttcaggctctagggctgtgctatccaatacagtaaccacatgcggctgtttaaagttaagccaattaaaatcacataagattaaaaattccttcctcagttgcactaaccacgtttctagaggcgtcactgtatgtagttcatggctactgtactgacagcgagagcatgtccatctgttggacagcactattctagagaactaaactggcttaacgagtcacagcctcagctgtgctgggacgacccttgtctccctgggtaggggggggggaatgggggagggctgatgaggccccagctggggcctgttgtctgggaccctccctctcctgagaggggaggcctggtggcttagcctgggcaggtcgtgtctcctcctgaccccagtggctgcggtgaggggaaccaccctcccttgctgcaccagtggccattagctcccgtcaccactgcaacccagggtcccagctggctgggtcctcttctgcccccagtgcccttccccttgggctgtgttggagtgagcacctcctctgtaggcacctctcacactgttgtctgttactgattttttttgataaaaagataataaaacctggtactttctaaa

SEQ ID NO: 383 - DNA, 239 bp, Homo sapiens
actcgccgcagcctgcgcgccttctccagtccgcggtgccatggcccccgcccgtctgttcgcgctgctgctgttcttcgtaggcggagtcgccgagtcgatccgagagactgaggtcatcgacccccaggacctcctagaaggccgatacttctccggagccctaccagacgatgaggatgtagtggggcccgggcaggaatctgatgactttgagctgtctggctctggagatctgg

SEQ ID NO: 384 - DNA, 150 bp, Homo sapiens
actaccccatcaactccaacttcaaagtgggctacaacactctcttttcctgtacctgtgtgccacctgacccagctacatctacatccaccactgggacaagccatcttgtaaaatgtgcggagaaggagaaaactttctgtgtgaatg

SEQ ID NO: 385 - DNA, 143 bp, Homo sapiens
ggcggcgcgccgggcagcgcttcggtggcggcggcggccgcggtggcagcgaaggcggcggcggcggcggcagtggcagtggccgctgcagccccacactccgccgccaaactggaggagcgacggaagccagaccccaggag

SEQ ID NO: 386 - DNA, 74 bp, Homo sapiens
gatggaggatgaagctgtcctggacagaggggcttccttcctcaagcatgtgtgtgatgaagaagaagtagaag

SEQ ID NO: 387 - DNA, 180 bp, Homo sapiens
gccaccataccatttacatcggagtccatgtgccgaagagttacaggagaaggagacgtcacaagagaaagacagggcacaaagaaaagaaggaaaaggagagaatctctgagaactactctgacaaatcagatattgaaaatgctgatgaatccagcagcagcatcctaaaacctctca

SEQ ID NO: 388 - DNA, 136 bp, Homo sapiens
tctctcctgctgcagaacgcatccgattcatcttgggagaggaggatgacagcccagctccccctcagctcttcacggaactggatgagctgctggccgtggatgggcaggagatggagtggaaggaaacagccag

SEQ ID NO: 389 - DNA, 161 bp, Homo sapiens
gtggatcaagtttgaagaaaaagtggaacagggtggggaaagatggagcaagccccatgtggccacattgtcccttcatagtttatttgagctgaggacatgtatggagaaaggatccatcatgcttgatcgggaggcttcttctctcccacagttggtgg

SEQ ID NO: 390 - DNA, 180 bp, Homo sapiens
agatgattgttgaccatcagattgagacaggcctattgaaacctgaacttaaggataaggtgacctatactttgctccggaagcaccggcatcaaaccaagaaatccaaccttcggtccctggctgacattgggaagacagtctccagtgcaagtaggatgtttaccaaccctgataatg

SEQ ID NO: 391 - DNA, 77 bp, Homo sapiens
gtagcccagccatgacccataggaatctgacttcctccagtctgaatgacatttctgataaaccggagaaggaccag

SEQ ID NO: 392 - DNA, 158 bp, Homo sapiens
ctgaagaataagttcatgaaaaaattgccacgtgatgcagaagcttccaacgtgcttgttggggaggttgactttttggatactcctttcattgcctttgttaggctacagcaggctgtcatgctgggtgccctgactgaagttcctgtgcccacaag

SEQ ID NO: 393 - DNA, 88 bp, Homo sapiens
gttcttgttcattctcttaggtcctaaggggaaagccaagtcctaccacgagattggcagagccattgccaccctgatgtctgatgag

SEQ ID NO: 394 - DNA, 155 bp, Homo sapiens
gtgttccatgacattgcttataaagcaaaagacaggcacgacctgattgctggtattgatgagttcctagatgaagtcatcgtccttccacctggggaatgggatccagcaattaggatagagcctcctaagagtcttccatcctctgacaaaag

SEQ ID NO: 395 - DNA, 114 bp, Homo sapiens
aaagaatatgtactcaggtggagagaatgttcagatgaatggggatacgccccatgatggaggtcacggaggaggaggacatggggattgtgaagaattgcagcgaactggacg

SEQ ID NO: 396 - DNA, 175 bp, Homo sapiens
gttctgtggtggactaattaaagacataaagaggaaagcgccattttttgccagtgatttttatgatgctttaaatattcaagctctttcggcaattctcttcatttatctggcaactgtaactaatgctatcacttttggaggactgcttggggatgccactgacaacatgcag

SEQ ID NO: 397 - DNA, 134 bp, Homo sapiens
ggcgtgttggagagtttcctgggcactgctgtctctggagccatcttttgcctttttgctggtcaaccactcactattctgagcagcaccggacctgtcctagtttttgagaggcttctatttaatttcagcaa

SEQ ID NO: 398 - DNA, 272 bp, Homo sapiens
ggacaataattttgactatttggagtttcgcctttggattggcctgtggtccgccttcctatgtctcattttggtagccactgatgccagcttcttggttcaatacttcacacgtttcacggaggagggcttttcctctctgattagcttcatctttatctatgatgctttcaagaagatgatcaagcttgcagattactaccccatcaactccaacttcaaagtgggctacaacactctcttttcctgtacctgtgtgccacctgacccag

SEQ ID NO: 399 - DNA, 71 bp, Homo sapiens
ctaatatctcaatatctaatgacaccacactggccccagagtatttgccaactatgtcttctactgacatg

SEQ ID NO: 400 - DNA, 192 bp, Homo sapiens
taccataatactacctttgactgggcatttttgtcgaagaaggagtgttcaaaatacggaggaaacctcgtcgggaacaactgtaattttgttcctgatatcacactcatgtcttttatcctcttcttgggaacctacacctcttccatggctctgaaaaaattcaaaactagtccttattttccaaccaca

SEQ ID NO: 401 - DNA, 114 bp, Homo sapiens
gcaagaaaactgatcagtgattttgccattatcttgtccattctcatcttttgtgtaatagatgccctagtaggcgtggacaccccaaaactaattgtgccaagtgagttcaag

SEQ ID NO: 402 - DNA, 162 bp, Homo sapiens
ccaacaagtccaaaccgaggttggttcgttccaccgtttggagaaaacccctggtgggtgtgccttgctgctgctatcccggctttgttggtcactatactgattttcatggaccaacaaattacagctgtgattgtaaacaggaaagaacataaactcaag

SEQ ID NO: 403 - DNA, 179 bp, Homo sapiens
aaaggagcagggtatcacttggatctcttttgggtggccatcctcatggttatatgctccctcatggctcttccgtggtatgtagctgctacggtcatctccattgctcacatcgacagtttgaagatggagacagagacttctgcacctggagaacaaccaaagtttctaggagtgag

SEQ ID NO: 404 - DNA, 72 bp, Homo sapiens
ggaacaaagagtcactggaacccttgtgtttattctgactggtctgtcagtctttatggctcccatcttgaa

SEQ ID NO: 405 - DNA, 70 bp, Homo sapiens
gtttatacccatgcctgtactctatggtgtgttcctgtatatgggagtagcatcccttaatggtgtgcag

SEQ ID NO: 406 - DNA, 174 bp, Homo sapiens
ttcatggatcgtctgaagctgcttctgatgcctctgaagcatcagcctgacttcatctacctgcgtcatgttcctctgcgcagagtccacctgttcactttcctgcaggtgttgtgtctggccctgctttggatcctcaagtcaacggtggctgctatcatttttccagtaatg

SEQ ID NO: 407 - DNA, 162 bp, Homo sapiens
atcttggcacttgtagctgtcagaaaaggcatggactacctcttctcccagcacgacctcagcttcctggatgatgtcattccagaaaaggacaagaaaaagaaggaggatgagaagaaaaagaaaaagaagaagggaagtctggacagtgacaatgatgat

SEQ ID NO: 408 - DNA, 97 bp, Homo sapiens
tctgactgcccatactcagaaaaagttccaagtattaaaattccaatggacatcatggaacagcaacctttcctaagcgatagcaaaccttctgaca

SEQ ID NO: 409 - DNA, 80 bp, Homo sapiens
gagaaagatcaccaacattccttgaacgccacacatcatgctgataaaattcctttccttcagtcactcggtatgccaag

SEQ ID NO: 410 - DNA, 4296 bp, Homo sapiens
tcctcctagaactccagtaaaagttgtgcctcaaattagaatagaacttgaacctgaagacaatgattatttctggaggagcaagggaacagaaactacattgtaacctgtttgtctttcttaaaactgacatttgttgttaatgtcatttgtttttgtttggctgtttgtttattttttaacttttatttcgtctcagtttttggtcacaggccaaataatacagcgctctctctgcttctctcttgcatagacacaatcaagacaatagtgcaccgttccttaaaaacagcatctgaggaatcccccttttgttcttaaactttcagatgtgtcctttgataaccaaattctgtcactcaagacacagacacgcacagaccctgtcctttgcctctattaagcagaggatggaagtattaaggattttgtaacaccttttatgaaaatgttgaaggaacttaaaactttagctttggagctgtgcttactggcttgtctttgtctggtagaacaaaccttgacctccagacagagtcccttctcacttatagagctctccaggactggaaaaagtgctgctattttaacttgctcttgcttgtaaatcctaatcttagagttatcaaaagaagaaaaaactgaaggtactttactccctatagagaaaccattgccatcattgtagcaagtgctggaatgtcccttttttcctatgcaactttttttaaccctttaatgaacttatctgttgagtacattgaagaatatttttcttcctagattttgttgtttaaattatggggcctaacctgccacttattttttgtcaatttttaaaacttttttttaattactgtaaagaaaatgaattttttcctgcagcaggaaacatagttttgagtagttctacctcttatttgtagctgccaggctttctgtaaaaattgtattgtatataatgtgatttttacacatacatacacacacaaatacacaatctctagggtaagccagaaggcaagatcagattaaaaacaccatgtttctaagcatccatttttccctttctttaaaagaaacttaactgttctatgaaggagattgagggagaagagacaaactcctatgtcatgagaataaccgatgttctgataatagtagcatctaggtacagatgctggttgtattaccacgtcaatgtcctatgcagtattgttagacattttctcattttgaaatatttgtgtgtttgtgtatgtgctctgtgccatggctggtgtatatatgtgcaatgttagaaggcaaaagagtgatggtaggcagagggcaaagtcattgaatctcttatgccagttttcataaaacccaaaccacatatgaaaaaatccattaagggtccaagaagtctgtccatatgaaaatgagggtaaatatagtttatttcccaggtatcagtcattataattgatataatagctctaacatgcaatataaaattcataggagtattaatagcccatttacacatctataaaatgtaatgggattgcagagctgcagagtacagtgtaacagtactctcatgcaatttttttcaggatgcaaaggcaattattctttgtaagcgggacatttagaatatatttgtgtacatattatatgtatgtatatttcaaagtaccacactgaaaattagacatttattaaccaaatttaacgtggtatttaaaggtaatatttttaatatgatacattacatattgtgaatgtatactaaaaaaacattttaaatgttaaaattataatttcagattcatataaccacaactgtgatatatcctaactataaccagttgttgaggggtatactagaagcagaatgaaaccacattttttggtttgataatatgcacttattgactcccactcattgttatgttaattaagttattattctgtctccttgtaattttgattacaaaaattttattatcctgagttagctgttacttttacagtacctgatactcctaaaacttttaacttatacaaattagtcaataatgaccccaattttttcattaaaataatagtggtgaattatatgttattgtgttaaaacctcacttgccaaattctggcttcacatttgtatttagggctatccttaaaatgatgagtctatattatctagctttctattaccctaatataaactggtataagaagactttccttttttctttatgcatggaagcatcaataaattgtttaaaaaccatgtatagtaaattcagcttaacccgtgatcttcttaagttaaaggtacttttgttttataaaagctctagataaaactttcttttctgatcatgaatcaagtatctgtggtttcatgcccctctctatacctttcaaagaactcctgaagcaacttaactcatcatttcagcctctgagtagaggtaaaacctatgtgtacttctgtttatgatccatattgatatttatgacatgaacacagaatagtaccttacatttgctaaacagacagttaatatcaaatcctttcaatattctgggaacccagggaagtttttaaaaatgtcattactttcaaaggaacagaagtagttaaccaaactaacaagcaaaacctgaggtttacctagtgacaccaaattatcggtattttaactgaatttacccattgactaagaatgaaccagatttggtggtggttttgtttctatgcaaactggacacaaattacaacagtaaatttttttataagtgcttctcccttctccatgatgtgacttccggagataaaggattcaaaagataaagacaaagtacgctcagagttgttaaccagaaagtcctggctgtggttgcagaaacactgttggaagaaaagagatgactaagtcaagtgtctgccttatcaaaagagcaaaaatgcctctggttttgtgtttgggagaaaagtatcttggacgcactgttttccttgataaaagtcatcttctctactgtgtgaaatgaatacttggaattctaattgttttgtgtgccaggggcagtaatgtccctgcctcttctcccaatcaaggttgaggagtggggctggggagaggacttaactgacttaagaagtaggaaaacaaaaacctctctcctcagccttccacctccaagagaggaggaaaaacagttgtctgctgtctgtaattcagtttgcgtgtattttatgctcatgcaccaacccatacagagtaaatcttttatcaactgtatactggtgtttaatagagaatgattgtcttccgagttttttggttccttttttaactgtgttaaagtacttgaaatgtattgactgctgactatattttaaaaacaaaatgaaataatttgagttgtattacagaggttgacattgttcagggatgggacaaagccttcttcaatccttttcatactacttaatgattttggtgcaggaacctgagattttctgatttatatttcatgatatttcacatttgctcttcacagcatgagcatgaagcccagtggcaccaaatggctgggtacaatcaagtgatattttgtagcacctcactatctgaaaggccatgagttttcagatgatttcattgagcttcattgcagcctgaaattttaaaaaagttgtgtaatacgccaaccagtcaagttgtgttttggccagagatttagatatgtccaatttcctggctcatttcattgtgctctatgggtacgtataaaaagcaagaattctgtttcctaggcaaacattgcaactcagggctaaagtcatccagtgaaacttttagagccagaagtaactttgtcccagtcctacaatgtgaaaagagtgaatagttgcctctttttagccattttcatggctggtacatattcgtacgcattacttttcagaatcaatacgcactttcagatattcttatttttattctcttaagtctttattaactttggagagagaaatgatgcatctttttattttaaatgaagtagatcaacatggtggaacaaaatgataaagaacagaaaacatttcaatatattactaataactttttccaatataaatcctaaaattcctataacatagtattttacagttttatgaagctttctattgtgacttttatggaattaagagatgaagaagatgagatattttagcatttatatttttcaaaattatatgtatacttaaaaataaagtaactttatgca

SEQ ID NO: 411 - DNA, 7716 bp, Homo sapiens
ggcggcgcgccgggcagcgcttcggtggcggcggcggccgcggtggcagcgaaggcggcggcggcggcggcagtggcagtggccgctgcagccccacactccgccgccaaactggaggagcgacggaagccagaccccaggaggatggaggatgaagctgtcctggacagaggggcttccttcctcaagcatgtgtgtgatgaagaagaagtagaaggccaccataccatttacatcggagtccatgtgccgaagagttacaggagaaggagacgtcacaagagaaagacagggcacaaagaaaagaaggaaaaggagagaatctctgagaactactctgacaaatcagatattgaaaatgctgatgaatccagcagcagcatcctaaaacctctcatctctcctgctgcagaacgcatccgattcatcttgggagaggaggatgacagcccagctccccctcagctcttcacggaactggatgagctgctggccgtggatgggcaggagatggagtggaaggaaacagccaggtggatcaagtttgaagaaaaagtggaacagggtggggaaagatggagcaagccccatgtggccacattgtcccttcatagtttatttgagctgaggacatgtatggagaaaggatccatcatgcttgatcgggaggcttcttctctcccacagttggtggagatgattgttgaccatcagattgagacaggcctattgaaacctgaacttaaggataaggtgacctatactttgctccggaagcaccggcatcaaaccaagaaatccaaccttcggtccctggctgacattgggaagacagtctccagtgcaagtaggatgtttaccaaccctgataatggtagcccagccatgacccataggaatctgacttcctccagtctgaatgacatttctgataaaccggagaaggaccagctgaagaataagttcatgaaaaaattgccacgtgatgcagaagcttccaacgtgcttgttggggaggttgactttttggatactcctttcattgcctttgttaggctacagcaggctgtcatgctgggtgccctgactgaagttcctgtgcccacaaggttcttgttcattctcttaggtcctaaggggaaagccaagtcctaccacgagattggcagagccattgccaccctgatgtctgatgaggtgttccatgacattgcttataaagcaaaagacaggcacgacctgattgctggtattgatgagttcctagatgaagtcatcgtccttccacctggggaatgggatccagcaattaggatagagcctcctaagagtcttccatcctctgacaaaagaaagaatatgtactcaggtggagagaatgttcagatgaatggggatacgccccatgatggaggtcacggaggaggaggacatggggattgtgaagaattgcagcgaactggacggttctgtggtggactaattaaagacataaagaggaaagcgccattttttgccagtgatttttatgatgctttaaatattcaagctctttcggcaattctcttcatttatctggcaactgtaactaatgctatcacttttggaggactgcttggggatgccactgacaacatgcagggcgtgttggagagtttcctgggcactgctgtctctggagccatcttttgcctttttgctggtcaaccactcactattctgagcagcaccggacctgtcctagtttttgagaggcttctatttaatttcagcaaggacaataattttgactatttggagtttcgcctttggattggcctgtggtccgccttcctatgtctcattttggtagccactgatgccagcttcttggttcaatacttcacacgtttcacggaggagggcttttcctctctgattagcttcatctttatctatgatgctttcaagaagatgatcaagcttgcagattactaccccatcaactccaacttcaaagtgggctacaacactctcttttcctgtacctgtgtgccacctgacccagctaatatctcaatatctaatgacaccacactggccccagagtatttgccaactatgtcttctactgacatgtaccataatactacctttgactgggcatttttgtcgaagaaggagtgttcaaaatacggaggaaacctcgtcgggaacaactgtaattttgttcctgatatcacactcatgtcttttatcctcttcttgggaacctacacctcttccatggctctgaaaaaattcaaaactagtccttattttccaaccacagcaagaaaactgatcagtgattttgccattatcttgtccattctcatcttttgtgtaatagatgccctagtaggcgtggacaccccaaaactaattgtgccaagtgagttcaagccaacaagtccaaaccgaggttggttcgttccaccgtttggagaaaacccctggtgggtgtgccttgctgctgctatcccggctttgttggtcactatactgattttcatggaccaacaaattacagctgtgattgtaaacaggaaagaacataaactcaagaaaggagcagggtatcacttggatctcttttgggtggccatcctcatggttatatgctccctcatggctcttccgtggtatgtagctgctacggtcatctccattgctcacatcgacagtttgaagatggagacagagacttctgcacctggagaacaaccaaagtttctaggagtgagggaacaaagagtcactggaacccttgtgtttattctgactggtctgtcagtctttatggctcccatcttgaagtttatacccatgcctgtactctatggtgtgttcctgtatatgggagtagcatcccttaatggtgtgcagttcatggatcgtctgaagctgcttctgatgcctctgaagcatcagcctgacttcatctacctgcgtcatgttcctctgcgcagagtccacctgttcactttcctgcaggtgttgtgtctggccctgctttggatcctcaagtcaacggtggctgctatcatttttccagtaatgatcttggcacttgtagctgtcagaaaaggcatggactacctcttctcccagcacgacctcagcttcctggatgatgtcattccagaaaaggacaagaaaaagaaggaggatgagaagaaaaagaaaaagaagaagggaagtctggacagtgacaatgatgattctgactgcccatactcagaaaaagttccaagtattaaaattccaatggacatcatggaacagcaacctttcctaagcgatagcaaaccttctgacagagaaagatcaccaacattccttgaacgccacacatcatgctgataaaattcctttccttcagtcactcggtatgccaagtcctcctagaactccagtaaaagttgtgcctcaaattagaatagaacttgaacctgaagacaatgattatttctggaggagcaagggaacagaaactacattgtaacctgtttgtctttcttaaaactgacatttgttgttaatgtcatttgtttttgtttggctgtttgtttattttttaacttttatttcgtctcagtttttggtcacaggccaaataatacagcgctctctctgcttctctcttgcatagacacaatcaagacaatagtgcaccgttccttaaaaacagcatctgaggaatcccccttttgttcttaaactttcagatgtgtcctttgataaccaaattctgtcactcaagacacagacacgcacagaccctgtcctttgcctctattaagcagaggatggaagtattaaggattttgtaacaccttttatgaaaatgttgaaggaacttaaaactttagctttggagctgtgcttactggcttgtctttgtctggtagaacaaaccttgacctccagacagagtcccttctcacttatagagctctccaggactggaaaaagtgctgctattttaacttgctcttgcttgtaaatcctaatcttagagttatcaaaagaagaaaaaactgaaggtactttactccctatagagaaaccattgccatcattgtagcaagtgctggaatgtcccttttttcctatgcaactttttttaaccctttaatgaacttatctgttgagtacattgaagaatatttttcttcctagattttgttgtttaaattatggggcctaacctgccacttattttttgtcaatttttaaaacttttttttaattactgtaaagaaaatgaattttttcctgcagcaggaaacatagttttgagtagttctacctcttatttgtagctgccaggctttctgtaaaaattgtattgtatataatgtgatttttacacatacatacacacacaaatacacaatctctagggtaagccagaaggcaagatcagattaaaaacaccatgtttctaagcatccatttttccctttctttaaaagaaacttaactgttctatgaaggagattgagggagaagagacaaactcctatgtcatgagaataaccgatgttctgataatagtagcatctaggtacagatgctggttgtattaccacgtcaatgtcctatgcagtattgttagacattttctcattttgaaatatttgtgtgtttgtgtatgtgctctgtgccatggctggtgtatatatgtgcaatgttagaaggcaaaagagtgatggtaggcagagggcaaagtcattgaatctcttatgccagttttcataaaacccaaaccacatatgaaaaaatccattaagggtccaagaagtctgtccatatgaaaatgagggtaaatatagtttatttcccaggtatcagtcattataattgatataatagctctaacatgcaatataaaattcataggagtattaatagcccatttacacatctataaaatgtaatgggattgcagagctgcagagtacagtgtaacagtactctcatgcaatttttttcaggatgcaaaggcaattattctttgtaagcgggacatttagaatatatttgtgtacatattatatgtatgtatatttcaaagtaccacactgaaaattagacatttattaaccaaatttaacgtggtatttaaaggtaatatttttaatatgatacattacatattgtgaatgtatactaaaaaaacattttaaatgttaaaattataatttcagattcatataaccacaactgtgatatatcctaactataaccagttgttgaggggtatactagaagcagaatgaaaccacattttttggtttgataatatgcacttattgactcccactcattgttatgttaattaagttattattctgtctccttgtaattttgattacaaaaattttattatcctgagttagctgttacttttacagtacctgatactcctaaaacttttaacttatacaaattagtcaataatgaccccaattttttcattaaaataatagtggtgaattatatgttattgtgttaaaacctcacttgccaaattctggcttcacatttgtatttagggctatccttaaaatgatgagtctatattatctagctttctattaccctaatataaactggtataagaagactttccttttttctttatgcatggaagcatcaataaattgtttaaaaaccatgtatagtaaattcagcttaacccgtgatcttcttaagttaaaggtacttttgttttataaaagctctagataaaactttcttttctgatcatgaatcaagtatctgtggtttcatgcccctctctatacctttcaaagaactcctgaagcaacttaactcatcatttcagcctctgagtagaggtaaaacctatgtgtacttctgtttatgatccatattgatatttatgacatgaacacagaatagtaccttacatttgctaaacagacagttaatatcaaatcctttcaatattctgggaacccagggaagtttttaaaaatgtcattactttcaaaggaacagaagtagttaaccaaactaacaagcaaaacctgaggtttacctagtgacaccaaattatcggtattttaactgaatttacccattgactaagaatgaaccagatttggtggtggttttgtttctatgcaaactggacacaaattacaacagtaaatttttttataagtgcttctcccttctccatgatgtgacttccggagataaaggattcaaaagataaagacaaagtacgctcagagttgttaaccagaaagtcctggctgtggttgcagaaacactgttggaagaaaagagatgactaagtcaagtgtctgccttatcaaaagagcaaaaatgcctctggttttgtgtttgggagaaaagtatcttggacgcactgttttccttgataaaagtcatcttctctactgtgtgaaatgaatacttggaattctaattgttttgtgtgccaggggcagtaatgtccctgcctcttctcccaatcaaggttgaggagtggggctggggagaggacttaactgacttaagaagtaggaaaacaaaaacctctctcctcagccttccacctccaagagaggaggaaaaacagttgtctgctgtctgtaattcagtttgcgtgtattttatgctcatgcaccaacccatacagagtaaatcttttatcaactgtatactggtgtttaatagagaatgattgtcttccgagttttttggttccttttttaactgtgttaaagtacttgaaatgtattgactgctgactatattttaaaaacaaaatgaaataatttgagttgtattacagaggttgacattgttcagggatgggacaaagccttcttcaatccttttcatactacttaatgattttggtgcaggaacctgagattttctgatttatatttcatgatatttcacatttgctcttcacagcatgagcatgaagcccagtggcaccaaatggctgggtacaatcaagtgatattttgtagcacctcactatctgaaaggccatgagttttcagatgatttcattgagcttcattgcagcctgaaattttaaaaaagttgtgtaatacgccaaccagtcaagttgtgttttggccagagatttagatatgtccaatttcctggctcatttcattgtgctctatgggtacgtataaaaagcaagaattctgtttcctaggcaaacattgcaactcagggctaaagtcatccagtgaaacttttagagccagaagtaactttgtcccagtcctacaatgtgaaaagagtgaatagttgcctctttttagccattttcatggctggtacatattcgtacgcattacttttcagaatcaatacgcactttcagatattcttatttttattctcttaagtctttattaactttggagagagaaatgatgcatctttttattttaaatgaagtagatcaacatggtggaacaaaatgataaagaacagaaaacatttcaatatattactaataactttttccaatataaatcctaaaattcctataacatagtattttacagttttatgaagctttctattgtgacttttatggaattaagagatgaagaagatgagatattttagcatttatatttttcaaaattatatgtatacttaaaaataaagtaactttatgca

SEQ ID NO: 412 - DNA, 2047 bp, Homo sapiens
ggcggcgcgccgggcagcgcttcggtggcggcggcggccgcggtggcagcgaaggcggcggcggcggcggcagtggcagtggccgctgcagccccacactccgccgccaaactggaggagcgacggaagccagaccccaggaggatggaggatgaagctgtcctggacagaggggcttccttcctcaagcatgtgtgtgatgaagaagaagtagaaggccaccataccatttacatcggagtccatgtgccgaagagttacaggagaaggagacgtcacaagagaaagacagggcacaaagaaaagaaggaaaaggagagaatctctgagaactactctgacaaatcagatattgaaaatgctgatgaatccagcagcagcatcctaaaacctctcatctctcctgctgcagaacgcatccgattcatcttgggagaggaggatgacagcccagctccccctcagctcttcacggaactggatgagctgctggccgtggatgggcaggagatggagtggaaggaaacagccaggtggatcaagtttgaagaaaaagtggaacagggtggggaaagatggagcaagccccatgtggccacattgtcccttcatagtttatttgagctgaggacatgtatggagaaaggatccatcatgcttgatcgggaggcttcttctctcccacagttggtggagatgattgttgaccatcagattgagacaggcctattgaaacctgaacttaaggataaggtgacctatactttgctccggaagcaccggcatcaaaccaagaaatccaaccttcggtccctggctgacattgggaagacagtctccagtgcaagtaggatgtttaccaaccctgataatggtagcccagccatgacccataggaatctgacttcctccagtctgaatgacatttctgataaaccggagaaggaccagctgaagaataagttcatgaaaaaattgccacgtgatgcagaagcttccaacgtgcttgttggggaggttgactttttggatactcctttcattgcctttgttaggctacagcaggctgtcatgctgggtgccctgactgaagttcctgtgcccacaaggttcttgttcattctcttaggtcctaaggggaaagccaagtcctaccacgagattggcagagccattgccaccctgatgtctgatgaggtgttccatgacattgcttataaagcaaaagacaggcacgacctgattgctggtattgatgagttcctagatgaagtcatcgtccttccacctggggaatgggatccagcaattaggatagagcctcctaagagtcttccatcctctgacaaaagaaagaatatgtactcaggtggagagaatgttcagatgaatggggatacgccccatgatggaggtcacggaggaggaggacatggggattgtgaagaattgcagcgaactggacggttctgtggtggactaattaaagacataaagaggaaagcgccattttttgccagtgatttttatgatgctttaaatattcaagctctttcggcaattctcttcatttatctggcaactgtaactaatgctatcacttttggaggactgcttggggatgccactgacaacatgcagggcgtgttggagagtttcctgggcactgctgtctctggagccatcttttgcctttttgctggtcaaccactcactattctgagcagcaccggacctgtcctagtttttgagaggcttctatttaatttcagcaaggacaataattttgactatttggagtttcgcctttggattggcctgtggtccgccttcctatgtctcattttggtagccactgatgccagcttcttggttcaatacttcacacgtttcacggaggagggcttttcctctctgattagcttcatctttatctatgatgctttcaagaagatgatcaagcttgcagattactaccccatcaactccaacttcaaagtgggctacaacactctcttttcctgtacctgtgtgccacctgacccag

SEQ ID NO: 413 - DNA, 150 bp, Homo sapiens
acaggaagcaccctaatgaggagtatgccaacgtgggcaccggggagctggcagcggaggtgctcatccagcaagctacatctacatccaccactgggacaagccatcttgtaaaatgtgcggagaaggagaaaactttctgtgtgaatg

SEQ ID NO: 414 - DNA, 187 bp, Homo sapiens
atccgccatgttggatgccgcagattcgccataacctcgccggctcttttcttaaaaaaataaaaataaaaagcgaagcgtcagcagggcgccccgccccctcggtcggcacgggagggggcccggaagagcccgaggcttttttttcctccgcggtggggcgttgccatggagacgcgggcggcag

SEQ ID NO: 415 - DNA, 177 bp, Homo sapiens
aaaacacggccatctttatgtgtaaatgttgtaacctcttctcaccaaatcagtcggaactcctctcccacgtttcagagaagcacatggaagaaggggttaatgttgatgagattattattccccttaggcctctgagtacacctgaaccccccaactcaagcaaaaccggagatg

SEQ ID NO: 416 - DNA, 252 bp, Homo sapiens
agtttttggtcatgaagaggaagagaggcaggcctaaggggtccacgaagaagtccagcacagaagaggagctggcagaaaacatcgtgagtccgactgaggacagcccgctggctccggaggaagggaacagcctgcctccaagcagcttggagtgtagcaagtgctgtcggaagttctccaacacgcgccagctgcggaagcacatctgcattatcgtgctgaatttgggtgaggaggaaggagaagcag

SEQ ID NO: 417 - DNA, 186 bp, Homo sapiens
gtaacgagtctgaccttgaactagaaaagaagtgtaaggaagatgatcgggaaaaagcctcgaaaagaccacggtcacagaaaacagagaaagtccagaagatctcaggaaaggaggccagacagctttctggggcgaagaaacccatcataagtgtggttttaactgcacacgaagcaattccag

SEQ ID NO: 418 - DNA, 151 bp, Homo sapiens
gtgctaccaagattgtgccagtggaggctgggccccctgaaacaggagctacaaattctgagaccacttcagcagacctggtgcctcggagaggctaccaggaatacgccattcagcagacaccttatgagcaaccaatgaagtcaagcag

SEQ ID NO: 419 - DNA, 1457 bp, Homo sapiens
gctaggtcccactcagctcaaaatcttcacttgtgaatactgcaacaaggtcttcaagttcaagcactcgctgcaggcccacctgaggatccacaccaatgaaaagccatacaagtgcccccagtgcagctatgccagtgccatcaaggccaacctcaatgtgcacctgcgcaagcacactggagagaagttcgcctgcgactattgctcgttcacctgcctgagcaagggccacctcaaggtgcacatcgagcgagtgcacaagaagatcaagcagcactgccgcttctgcaagaagaagtactctgacgtcaagaacctcatcaagcacatccgagacgcgcatgacccacaggacaagaaggtcaaagaggccttggacgagctctgcctgatgacgagggagggcaagcggcagctgctctatgactgccacatctgtgagcgcaagttcaagaacgagctggaccgtgaccgccatatgctggtccacggagacaagtggccttttgcctgtgagctctgtggccatggggccaccaagtaccaggcgctggaactgcatgtcaggaagcaccccttcgtgtacgtctgtgccgtctgccgcaagaagttcgtcagctccatcaggctgcgcacccacatcaaagaggtgcacggggctgcccaggaggccttggtcttcaccagttccatcaaccagagcttctgcctcctggaacctggtggggacatccagcaagaagctctgggggaccagctacagctggtggaagaggagtttgccctccagggcgtgaatgcactcaaggaagaggcctgtcctggggacactcagctggaggagggccggaaggagccggaggcccctggggaaatgcctgccccagctgtgcacctggcctccccgcaggccgaaagcacagccctgccaccctgtgagctggaaaccaccgtggtctcctcctcagacctgcattctcaagaggtggtttcagatgattttttgttgaaaaatgatacctcctccgcagaggctcatgctgctcctgagaagcccccagacatgcagcacagaagctcagtccagacgcaaggtgaagtgatcacactactgctgtccaaggcccagagtgctgggtcagatcaggaaagccatggcgcccagagccccctaggggaagggcagaacatggctgtgctttcagctggtgacccagatcccagcaggtgtctcaggtcaaacccagctgaggcctcagacctcctccctccagtagctggtggtggggacaccatcacacatcagcctgactcttgcaaagctgcccctgagcaccggtcaggcatcaccgctttcatgaaggtcctgaacagtttacagaagaagcaaatgaacaccagcttgtgtgagcggatccggaaggtttatggagacctggagtgtgaatactgtg

SEQ ID NO: 420 - DNA, 233 bp, Homo sapiens
gcaaacttttttggtaccaagtgcattttgatatgcatgtccgcacccacacccgggaacatctgtattattgctctcagtgtcattattcttccatcaccaaaaactgccttaaacgccacgtaattcagaaacacagtaacatcttgctgaagtgtcccaccgatggctgtgactactcaactccagataaatataagctacaggcacatcttaaagttcacacagcactg

SEQ ID NO: 421 - DNA, 88 bp, Homo sapiens
gacaaaaggagttattcttgtcctgtttgtgaaaagtctttttcagaggatcgattgataaagtcacatatcaagaccaaccatcctg

SEQ ID NO: 422 - DNA, 150 bp, Homo sapiens
aggtctccatgagcaccatttctgaggttctcgggaggagggttcagctgaaagggctaattggaaagagagccatgaaatgcccatattgtgacttttatttcatgaagaatggctcagaccttcagcgtcatatttgggctcatgaag

SEQ ID NO: 423 - DNA, 174 bp, Homo sapiens
gtgtgaagcccttcaagtgttctttgtgtgagtatgcaactcgtagcaagagtaacctcaaggctcatatgaatcgtcacagcactgagaaaacccacctatgtgacatgtgtggcaagaaattcaaatcaaaagggacactgaaaagtcacaaactccttcacactgcagatg

SEQ ID NO: 424 - DNA, 89 bp, Homo sapiens
ggaagcagtttaagtgcacggtgtgtgactacacagcggcccagaagccacagctgctgcggcacatggaacagcatgtctccttcaag

SEQ ID NO: 425 - DNA, 139 bp, Homo sapiens
cctttccgctgtgcccattgccattactcctgcaacatatctggctctctgaagcggcactacaacaggaagcaccctaatgaggagtatgccaacgtgggcaccggggagctggcagcggaggtgctcatccagcaag

SEQ ID NO: 426 - DNA, 119 bp, Homo sapiens
gtggtttgaagtgtcctgtttgcagctttgtatatggcaccaaatgggagttcaataggcacttgaagaacaaacatggcttgaaggtggtggaaattgatggagaccccaagtgggag

SEQ ID NO: 427 - DNA, 127 bp, Homo sapiens
acagcaacagaagctcctgaggagccctccacccagtatctccacatcacagaggccgaagaagacgttcaagggacacaggcagcggtggccgcgctccaggacctgagatacacctctgagagtg

SEQ ID NO: 428 - DNA, 131 bp, Homo sapiens
gcgaccgactggaccccacggccgtgaacatcctgcagcagatcattgagctgggcgccgagacccatgacgccactgcccttgcctcggtggttgccatggcaccagggacggtgactgtggttaagcag

SEQ ID NO: 429 - DNA, 934 bp, Homo sapiens
gtcaccgaggaggagcccagctccaaccacacggtcatgatccaggagacggtccagcaagcgtccgtggagcttgccgagcagcaccacctggtggtgtcctccgacgacgtggagggcattgagacggtgactgtctacacgcagggcggggaggcctcggagttcatcgtctacgtgcaggaggccatgcagcctgtggaggagcaggctgtggagcagccggcccaggaactctagaggacatgtggcatcggatggccacagggcggggctgccaggctctgcaggcacccagggtggggaggccacccttcctgccctacccgcagaatggtgctctcctttgccctccctgcccagcagcctgataggactctcctagtccaacttggggtgggcaaggcagtcagcatcaccagcaataccacaggaccctcaccccagcatagacacacaccccctgacccttaccatctgcttcctgaaagacttcagtgtcagctcccctacacacaccccacaccttcaccccttgcttcaagattcaaacagagactcccagtccccctcagcatcttccctgaatcacagccccagctccttgacccccatctaggtgccaaatgttcatctgcaaccgctatgcagtctggtgagagggagacagccatcacatagaaagtgaccgtacgggtttttaatcactgctgggtggggtgggggtagggggattgtcctggctttgtcgacaaagtcccacttccccgagtattaagggcccttggtatcaagtgaggtaaattcacccatcacagggtctcgccctaccatcctggaattatttcacttttaagataaatgcactatttcactgttcgcctcccattctaaggaggtgaggtggttggaataaaaacagttcctgtctgaa

SEQ ID NO: 430 - DNA, 4594 bp, Homo sapiens
atccgccatgttggatgccgcagattcgccataacctcgccggctcttttcttaaaaaaataaaaataaaaagcgaagcgtcagcagggcgccccgccccctcggtcggcacgggagggggcccggaagagcccgaggcttttttttcctccgcggtggggcgttgccatggagacgcgggcggcagaaaacacggccatctttatgtgtaaatgttgtaacctcttctcaccaaatcagtcggaactcctctcccacgtttcagagaagcacatggaagaaggggttaatgttgatgagattattattccccttaggcctctgagtacacctgaaccccccaactcaagcaaaaccggagatgagtttttggtcatgaagaggaagagaggcaggcctaaggggtccacgaagaagtccagcacagaagaggagctggcagaaaacatcgtgagtccgactgaggacagcccgctggctccggaggaagggaacagcctgcctccaagcagcttggagtgtagcaagtgctgtcggaagttctccaacacgcgccagctgcggaagcacatctgcattatcgtgctgaatttgggtgaggaggaaggagaagcaggtaacgagtctgaccttgaactagaaaagaagtgtaaggaagatgatcgggaaaaagcctcgaaaagaccacggtcacagaaaacagagaaagtccagaagatctcaggaaaggaggccagacagctttctggggcgaagaaacccatcataagtgtggttttaactgcacacgaagcaattccaggtgctaccaagattgtgccagtggaggctgggccccctgaaacaggagctacaaattctgagaccacttcagcagacctggtgcctcggagaggctaccaggaatacgccattcagcagacaccttatgagcaaccaatgaagtcaagcaggctaggtcccactcagctcaaaatcttcacttgtgaatactgcaacaaggtcttcaagttcaagcactcgctgcaggcccacctgaggatccacaccaatgaaaagccatacaagtgcccccagtgcagctatgccagtgccatcaaggccaacctcaatgtgcacctgcgcaagcacactggagagaagttcgcctgcgactattgctcgttcacctgcctgagcaagggccacctcaaggtgcacatcgagcgagtgcacaagaagatcaagcagcactgccgcttctgcaagaagaagtactctgacgtcaagaacctcatcaagcacatccgagacgcgcatgacccacaggacaagaaggtcaaagaggccttggacgagctctgcctgatgacgagggagggcaagcggcagctgctctatgactgccacatctgtgagcgcaagttcaagaacgagctggaccgtgaccgccatatgctggtccacggagacaagtggccttttgcctgtgagctctgtggccatggggccaccaagtaccaggcgctggaactgcatgtcaggaagcaccccttcgtgtacgtctgtgccgtctgccgcaagaagttcgtcagctccatcaggctgcgcacccacatcaaagaggtgcacggggctgcccaggaggccttggtcttcaccagttccatcaaccagagcttctgcctcctggaacctggtggggacatccagcaagaagctctgggggaccagctacagctggtggaagaggagtttgccctccagggcgtgaatgcactcaaggaagaggcctgtcctggggacactcagctggaggagggccggaaggagccggaggcccctggggaaatgcctgccccagctgtgcacctggcctccccgcaggccgaaagcacagccctgccaccctgtgagctggaaaccaccgtggtctcctcctcagacctgcattctcaagaggtggtttcagatgattttttgttgaaaaatgatacctcctccgcagaggctcatgctgctcctgagaagcccccagacatgcagcacagaagctcagtccagacgcaaggtgaagtgatcacactactgctgtccaaggcccagagtgctgggtcagatcaggaaagccatggcgcccagagccccctaggggaagggcagaacatggctgtgctttcagctggtgacccagatcccagcaggtgtctcaggtcaaacccagctgaggcctcagacctcctccctccagtagctggtggtggggacaccatcacacatcagcctgactcttgcaaagctgcccctgagcaccggtcaggcatcaccgctttcatgaaggtcctgaacagtttacagaagaagcaaatgaacaccagcttgtgtgagcggatccggaaggtttatggagacctggagtgtgaatactgtggcaaacttttttggtaccaagtgcattttgatatgcatgtccgcacccacacccgggaacatctgtattattgctctcagtgtcattattcttccatcaccaaaaactgccttaaacgccacgtaattcagaaacacagtaacatcttgctgaagtgtcccaccgatggctgtgactactcaactccagataaatataagctacaggcacatcttaaagttcacacagcactggacaaaaggagttattcttgtcctgtttgtgaaaagtctttttcagaggatcgattgataaagtcacatatcaagaccaaccatcctgaggtctccatgagcaccatttctgaggttctcgggaggagggttcagctgaaagggctaattggaaagagagccatgaaatgcccatattgtgacttttatttcatgaagaatggctcagaccttcagcgtcatatttgggctcatgaaggtgtgaagcccttcaagtgttctttgtgtgagtatgcaactcgtagcaagagtaacctcaaggctcatatgaatcgtcacagcactgagaaaacccacctatgtgacatgtgtggcaagaaattcaaatcaaaagggacactgaaaagtcacaaactccttcacactgcagatgggaagcagtttaagtgcacggtgtgtgactacacagcggcccagaagccacagctgctgcggcacatggaacagcatgtctccttcaagcctttccgctgtgcccattgccattactcctgcaacatatctggctctctgaagcggcactacaacaggaagcaccctaatgaggagtatgccaacgtgggcaccggggagctggcagcggaggtgctcatccagcaaggtggtttgaagtgtcctgtttgcagctttgtatatggcaccaaatgggagttcaataggcacttgaagaacaaacatggcttgaaggtggtggaaattgatggagaccccaagtgggagacagcaacagaagctcctgaggagccctccacccagtatctccacatcacagaggccgaagaagacgttcaagggacacaggcagcggtggccgcgctccaggacctgagatacacctctgagagtggcgaccgactggaccccacggccgtgaacatcctgcagcagatcattgagctgggcgccgagacccatgacgccactgcccttgcctcggtggttgccatggcaccagggacggtgactgtggttaagcaggtcaccgaggaggagcccagctccaaccacacggtcatgatccaggagacggtccagcaagcgtccgtggagcttgccgagcagcaccacctggtggtgtcctccgacgacgtggagggcattgagacggtgactgtctacacgcagggcggggaggcctcggagttcatcgtctacgtgcaggaggccatgcagcctgtggaggagcaggctgtggagcagccggcccaggaactctagaggacatgtggcatcggatggccacagggcggggctgccaggctctgcaggcacccagggtggggaggccacccttcctgccctacccgcagaatggtgctctcctttgccctccctgcccagcagcctgataggactctcctagtccaacttggggtgggcaaggcagtcagcatcaccagcaataccacaggaccctcaccccagcatagacacacaccccctgacccttaccatctgcttcctgaaagacttcagtgtcagctcccctacacacaccccacaccttcaccccttgcttcaagattcaaacagagactcccagtccccctcagcatcttccctgaatcacagccccagctccttgacccccatctaggtgccaaatgttcatctgcaaccgctatgcagtctggtgagagggagacagccatcacatagaaagtgaccgtacgggtttttaatcactgctgggtggggtgggggtagggggattgtcctggctttgtcgacaaagtcccacttccccgagtattaagggcccttggtatcaagtgaggtaaattcacccatcacagggtctcgccctaccatcctggaattatttcacttttaagataaatgcactatttcactgttcgcctcccattctaaggaggtgaggtggttggaataaaaacagttcctgtctgaa

SEQ ID NO: 431 - DNA, 3283 bp, Homo sapiens
atccgccatgttggatgccgcagattcgccataacctcgccggctcttttcttaaaaaaataaaaataaaaagcgaagcgtcagcagggcgccccgccccctcggtcggcacgggagggggcccggaagagcccgaggcttttttttcctccgcggtggggcgttgccatggagacgcgggcggcagaaaacacggccatctttatgtgtaaatgttgtaacctcttctcaccaaatcagtcggaactcctctcccacgtttcagagaagcacatggaagaaggggttaatgttgatgagattattattccccttaggcctctgagtacacctgaaccccccaactcaagcaaaaccggagatgagtttttggtcatgaagaggaagagaggcaggcctaaggggtccacgaagaagtccagcacagaagaggagctggcagaaaacatcgtgagtccgactgaggacagcccgctggctccggaggaagggaacagcctgcctccaagcagcttggagtgtagcaagtgctgtcggaagttctccaacacgcgccagctgcggaagcacatctgcattatcgtgctgaatttgggtgaggaggaaggagaagcaggtaacgagtctgaccttgaactagaaaagaagtgtaaggaagatgatcgggaaaaagcctcgaaaagaccacggtcacagaaaacagagaaagtccagaagatctcaggaaaggaggccagacagctttctggggcgaagaaacccatcataagtgtggttttaactgcacacgaagcaattccaggtgctaccaagattgtgccagtggaggctgggccccctgaaacaggagctacaaattctgagaccacttcagcagacctggtgcctcggagaggctaccaggaatacgccattcagcagacaccttatgagcaaccaatgaagtcaagcaggctaggtcccactcagctcaaaatcttcacttgtgaatactgcaacaaggtcttcaagttcaagcactcgctgcaggcccacctgaggatccacaccaatgaaaagccatacaagtgcccccagtgcagctatgccagtgccatcaaggccaacctcaatgtgcacctgcgcaagcacactggagagaagttcgcctgcgactattgctcgttcacctgcctgagcaagggccacctcaaggtgcacatcgagcgagtgcacaagaagatcaagcagcactgccgcttctgcaagaagaagtactctgacgtcaagaacctcatcaagcacatccgagacgcgcatgacccacaggacaagaaggtcaaagaggccttggacgagctctgcctgatgacgagggagggcaagcggcagctgctctatgactgccacatctgtgagcgcaagttcaagaacgagctggaccgtgaccgccatatgctggtccacggagacaagtggccttttgcctgtgagctctgtggccatggggccaccaagtaccaggcgctggaactgcatgtcaggaagcaccccttcgtgtacgtctgtgccgtctgccgcaagaagttcgtcagctccatcaggctgcgcacccacatcaaagaggtgcacggggctgcccaggaggccttggtcttcaccagttccatcaaccagagcttctgcctcctggaacctggtggggacatccagcaagaagctctgggggaccagctacagctggtggaagaggagtttgccctccagggcgtgaatgcactcaaggaagaggcctgtcctggggacactcagctggaggagggccggaaggagccggaggcccctggggaaatgcctgccccagctgtgcacctggcctccccgcaggccgaaagcacagccctgccaccctgtgagctggaaaccaccgtggtctcctcctcagacctgcattctcaagaggtggtttcagatgattttttgttgaaaaatgatacctcctccgcagaggctcatgctgctcctgagaagcccccagacatgcagcacagaagctcagtccagacgcaaggtgaagtgatcacactactgctgtccaaggcccagagtgctgggtcagatcaggaaagccatggcgcccagagccccctaggggaagggcagaacatggctgtgctttcagctggtgacccagatcccagcaggtgtctcaggtcaaacccagctgaggcctcagacctcctccctccagtagctggtggtggggacaccatcacacatcagcctgactcttgcaaagctgcccctgagcaccggtcaggcatcaccgctttcatgaaggtcctgaacagtttacagaagaagcaaatgaacaccagcttgtgtgagcggatccggaaggtttatggagacctggagtgtgaatactgtggcaaacttttttggtaccaagtgcattttgatatgcatgtccgcacccacacccgggaacatctgtattattgctctcagtgtcattattcttccatcaccaaaaactgccttaaacgccacgtaattcagaaacacagtaacatcttgctgaagtgtcccaccgatggctgtgactactcaactccagataaatataagctacaggcacatcttaaagttcacacagcactggacaaaaggagttattcttgtcctgtttgtgaaaagtctttttcagaggatcgattgataaagtcacatatcaagaccaaccatcctgaggtctccatgagcaccatttctgaggttctcgggaggagggttcagctgaaagggctaattggaaagagagccatgaaatgcccatattgtgacttttatttcatgaagaatggctcagaccttcagcgtcatatttgggctcatgaaggtgtgaagcccttcaagtgttctttgtgtgagtatgcaactcgtagcaagagtaacctcaaggctcatatgaatcgtcacagcactgagaaaacccacctatgtgacatgtgtggcaagaaattcaaatcaaaagggacactgaaaagtcacaaactccttcacactgcagatgggaagcagtttaagtgcacggtgtgtgactacacagcggcccagaagccacagctgctgcggcacatggaacagcatgtctccttcaagcctttccgctgtgcccattgccattactcctgcaacatatctggctctctgaagcggcactacaacaggaagcaccctaatgaggagtatgccaacgtgggcaccggggagctggcagcggaggtgctcatccagcaag

SEQ ID NO: 432 - DNA, 150 bp, Homo sapiens
gcctcatcccctcttcagtgcaggaatatgttagcgttgtatcttgggagaaagacacagtctccatcatcccagccttgcctccccgattgaaagagatgaaaagccaggaatcggctgcaggttccaaactagtccttcggtgtgaaa

SEQ ID NO: 433 - DNA, 183 bp, Homo sapiens
agccgagcgctgggctgaggagcagagagagcggggcgccgagtgcgggcggctgggagcgcgctgagcgggggagaggcgctgccgcacggccggccacaggaccacctccccggagaatagggcctctttatggcatgtggctggtaactttcctcctgctcctggactctttacacaaag

SEQ ID NO: 434 - DNA, 318 bp, Homo sapiens
cccgccctgaagatgttggcaccagcctctactttgtaaatgactccttgcagcaggtgaccttttccagctccgtgggggtggtggtgccctgcccggccgcgggctcccccagcgcggcccttcgatggtacctggccacaggggacgacatctacgacgtgccgcacatccggcacgtccacgccaacgggacgctgcagctctaccccttctccccctccgccttcaatagctttatccacgacaatgactacttctgcaccgcggagaacgctgccggcaagatccggagccccaacatccgcgtcaaagcag

SEQ ID NO: 435 - DNA, 147 bp, Homo sapiens
ttttcagggaaccctacaccgtccgggtggaggatcaaaggtcaatgcgtggcaacgtggccgtcttcaagtgcctcatcccctcttcagtgcaggaatatgttagcgttgtatcttgggagaaagacacagtctccatcatcccag

SEQ ID NO: 436 - DNA, 147 bp, Homo sapiens
aacacaggttttttattacctaccacggcgggctgtacatctctgacgtacagaaggaggacgccctctccacctatcgctgcatcaccaagcacaagtatagcggggagacccggcagagcaatggggcacgcctctctgtgacag

SEQ ID NO: 437 - DNA, 279 bp, Homo sapiens
accctgctgagtcgatccccaccatcctggatggcttccactcccaggaagtgtgggccggccacaccgtggagctgccctgcaccgcctcgggctaccctatccccgccatccgctggctcaaggatggccggcccctcccggctgacagccgctggaccaagcgcatcacagggctgaccatcagcgacttgcggaccgaggacagcggcacctacatttgtgaggtcaccaacaccttcggttcggcagaggccacaggcatcctcatggtcattg

SEQ ID NO: 438 - DNA, 276 bp, Homo sapiens
atccccttcatgtgaccctgacaccaaagaagctgaagaccggcattggcagcacggtcatcctctcctgtgccctgacgggctccccagagttcaccatccgctggtatcgcaacacggagctggtgctgcctgacgaggccatctccatccgcgggctcagcaacgagacgctgctcatcacctcggcccagaagagccattccggggcctaccagtgcttcgctacccgcaaggcccagaccgcccaggactttgccatcattgcacttgagg

SEQ ID NO: 439 - DNA, 297 bp, Homo sapiens
atggcacgccccgcatcgtctcgtccttcagcgagaaggtggtcaaccccggggagcagttctcactgatgtgtgcggccaagggcgccccgccccccacggtcacctgggccctcgacgatgagcccatcgtgcgggatggcagccaccgcaccaaccagtacaccatgtcggacggcaccaccatcagccacatgaacgtcacaggcccccagatccgcgacgggggcgtgtaccggtgcacagcgcggaacttggtgggcagtgctgaatatcaggcgcgaataaacgtaagag

SEQ ID NO: 440 - DNA, 273 bp, Homo sapiens
gcccacccagcatccgggctatgcggaacatcacagcagtcgccgggcgggacacccttatcaactgcagggtcatcggctatccctactactccatcaagtggtacaaggatgccctgctgctgccagacaaccaccgccaggtggtgtttgagaatgggaccctcaagctgactgacgtgcagaagggcatggatgagggggagtacctgtgcagtgtcctcatccagccccagctctccatcagccagagcgttcacgtagccgtcaaag

SEQ ID NO: 441 - DNA, 279 bp, Homo sapiens
tgccccctctgatccagcccttcgaattcccacccgcctccatcggccagctgctctacattccctgtgtggtgtcctcgggggacatgcccatccgtatcacctggaggaaggacggacaggtgatcatctcaggctcgggcgtgaccatcgagagcaaggaattcatgagctccctgcagatctctagcgtctccctcaagcacaacggcaactatacatgcatcgccagcaacgcagccgccaccgtgagccgggagcgccagctcatcgtgcgtg

SEQ ID NO: 442 - DNA, 119 bp, Homo sapiens
tgccccctcgatttgtggtgcaacccaacaaccaggatggcatctacggcaaagctggtgtgctcaactgctcggtggacggctaccccccacccaaggtcatgtggaagcatgccaag

SEQ ID NO: 443 - DNA, 178 bp, Homo sapiens
gggagcgggaacccccagcagtaccaccctgtgcccctcactggccgcatccagatcctgcccaacagctcgctgctgatccgccacgtcctagaagaggacatcggctactacctctgccaggccagcaacggcgtaggcaccgacatcagcaagtccatgttcctcacagtcaaga

SEQ ID NO: 444 - DNA, 200 bp, Homo sapiens
tcccggccatgatcacttcccaccccaacaccaccatcgccatcaagggccatgcgaaggagctaaactgcacggcacggggtgagcggcccatcatcatccgctgggagaagggggacacagtcatcgaccctgaccgcgtcatgcggtatgccatcgccaccaaggacaacggcgacgaggtcgtctccacactgaag

SEQ ID NO: 445 - DNA, 97 bp, Homo sapiens
ctcaagcccgctgaccgtggggactctgtgttcttcagctgccatgccatcaactcgtatggggaggaccggggcttgatccaactcactgtgcaag

SEQ ID NO: 446 - DNA, 129 bp, Homo sapiens
agccccccgaccccccagagctggagatccgggaggtgaaggcccggagcatgaacctgcgctggacccagcgattcgacgggaacagcatcatcacgggcttcgacattgaatacaagaacaaatcag

SEQ ID NO: 447 - DNA, 168 bp, Homo sapiens
attcctgggacttcaagcagtccacacgcaacatctcccccaccatcaaccaggccaacattgtggacttgcacccggcatctgtgtacagcatccgcatgtactctttcaacaagattggccgcagtgaaccaagcaaggagctcaccatcagcactgaggaggccg

SEQ ID NO: 448 - DNA, 71 bp, Homo sapiens
ctcccgatgggccccccatggatgttaccttgcagccagtgacctcacagagcatccaggtgacctggaag

SEQ ID NO: 449 - DNA, 241 bp, Homo sapiens
gcacccaagaaggagctgcagaacggtgtcatccggggctaccagattggctacagagagaacagccccggcagcaacgggcagtacagcatcgtggagatgaaggccacgggggacagcgaggtctacaccctggacaacctcaagaagttcgcccagtatggggtggtggtccaagccttcaatcgggctggcacggggccctcttccagcgagatcaatgccaccactctggaggatg

SEQ ID NO: 450 - DNA, 147 bp, Homo sapiens
tgcccagccagccccctgagaacgtccgggccctgtccatcacttctgacgtggccgtcatctcctggtcagagcccccgcgcagcaccctcaatggcgtcctcaaaggctatcgggtcatcttctggtccctctatgttgatgggg

SEQ ID NO: 451 - DNA, 156 bp, Homo sapiens
agtggggcgagatgcagaacatcaccaccacgcgggagcgggtggagctgcggggcatggagaagttcaccaactacagcgtccaggtgctggcctacacccaggctggggacggcgtacgcagcagtgtgctctacatccagaccaaggaggacg

SEQ ID NO: 452 - DNA, 140 bp, Homo sapiens
ttccaggtccccctgctggcatcaaagctgtcccttcatcagctagcagtgtggttgtgtcttggctcccccctaccaagcccaacggggtgatccgcaagtacaccatcttctgttccagccccgggtctggccagccg

SEQ ID NO: 453 - DNA, 154 bp, Homo sapiens
gctcccagcgagtacgagacgagtccagagcagctcttctaccggatcgcccacctaaaccgcggtcagcagtatctgctgtgggtggccgccgtcacctctgccggccggggcaacagcagcgagaaggtgaccatcgagcctgctggcaagg

SEQ ID NO: 454 - DNA, 118 bp, Homo sapiens
ccccagcaaagatcatctcctttgggggcaccgtgacaacaccttggatgaaagatgttcggctgccttgcaattcagtgggagatccagcccctgctgtgaagtggaccaaggacag

SEQ ID NO: 455 - DNA, 164 bp, Homo sapiens
tgaagactcggccattccagtgtccatggatgggcaccggctcatccacaccaatggcacactgctgctgcgtgcagtgaaggctgaggactctggctactacacgtgcacggccaccaacactggtggctttgacaccatcatcgtcaaccttctggtgcaag

SEQ ID NO: 456 - DNA, 99 bp, Homo sapiens
ttcccccggaccagccccgcctcactgtctccaaaacctcagcttcgtccatcaccctgacctggattccaggtgacaatgggggcagctccatccgag

SEQ ID NO: 457 - DNA, 189 bp, Homo sapiens
gcttcgtgctacagtactcggtggacaacagcgaggagtggaaggatgtgttcatcagctccagcgagcgctccttcaagctggacagcctcaagtgtggcacgtggtacaaggtgaagctggcagccaagaacagcgtgggctctgggcgcatcagcgagatcatcgaggccaagacccacgggcggg

SEQ ID NO: 458 - DNA, 288 bp, Homo sapiens
agccctccttcagcaaagaccaacacctcttcacccacatcaactccacgcatgctcggcttaacctgcagggctggaacaatgggggctgccctatcacagccatcgttctggagtaccggcccaaggggacctgggcctggcagggcctccgggccaacagctccggggaggtgtttctgacggaactgcgagaggccacgtggtacgagctgcgcatgagggcttgcaacagtgcgggctgcggcaatgaaacagcccagttcgccaccctggactacgatggca

SEQ ID NO: 459 - DNA, 156 bp, Homo sapiens
gcaccattccacccatcaagtctgctcaaggtgaaggggatgatgtgaagaagctgttcaccatcggctgccctgtcatcctggccacactgggggtggcactgctcttcatcgtacgcaagaagaggaaggagaaacggctgaagcgactccgag

SEQ ID NO: 460 - DNA, 31 bp, Homo sapiens
atgcaaagagtttggcagaaatgttgataag

SEQ ID NO: 461 - DNA, 119 bp, Homo sapiens
caagaacaatagaagctttgacacccctgtgaaagggccaccccagggcccacggctacacattgacatccccagggtccagctgctcatcgaggacaaagaaggcatcaagcaactgg

SEQ ID NO: 462 - DNA, 153 bp, Homo sapiens
gagatgacaaggccaccatccctgtgacagatgctgagttcagccaagctgtcaacccacagagcttctgtactggcgtctccttgcaccacccaaccctcatccagagcacaggacccctcatcgacatgtctgacatccggccaggaacca

SEQ ID NO: 463 - DNA, 195 bp, Homo sapiens
atccagtgtccaggaagaatgtgaagtcagcccacagcacccggaaccggtactcaagccagtggaccctgaccaagtgccaggcctccacacctgcccgcaccctcacctccgactggcgcaccgtgggctcccagcatggtgtcacggtcactgagagtgacagctacagtgccagcctgtcccaggacacag

SEQ ID NO: 464 - DNA, 312 bp, Homo sapiens
acaaaggaaggaacagcatggtgtccactgagagtgcctcttccacctacgaggagctggcccgggcctatgagcatgccaagctggaggagcagctgcagcacgccaagtttgagatcaccgagtgcttcatctctgacagttcctctgaccagatgaccacaggcaccaacgagaacgccgacagcatgacatccatgagcacaccctcagagcctggcatctgccgctttaccgcctcaccacccaagccccaggatgcggaccggggcaaaaacgtggctgtgcccatccctcaccgggccaacaaga

SEQ ID NO: 465 - DNA, 1032 bp, Homo sapiens
gtgactactgcaacctgcccctgtatgccaagtcagaggccttctttcgaaaggcagatggacgtgagccctgccccgtggtcccaccccgtgaggcctccatccggaacctggctcgaacctaccacacccaggctcgccacctgaccctggaccctgccagcaagtccttgggccttccccacccaggggcccccgctgccgcctccacagccaccttacctcagaggactctggccatgccagcccccccagccggcacagcccccccagcccccggccccacccctgctgagccacccaccgcccccagcgctgcccctccggcccccagcaccgagcctccacgagccgggggcccacacaccaaaatggggggctccagggactcgcttctcgagatgagcacatcgggggtagggaggtctcagaagcagggggccggggcctactccaaatcctacaccctggtgtagggcccgcaggaagagcagccacgcctggaccgcgccgcgccgcagccccacacgccagctcggctgtttttctgcattatttatattcaactgacagacaaaaaccaaccaacgacaaaacaaaaacccccaatcatgaacgcctgtacatagaactcttttgtacaaatgaaactattttcttcttctccatgaagccagggcacaaagaatttgacagtacaagtcaaatcccccaccccacaaaatatgtgtggagatatatatacatatatagacagacaggaacgcgtccacgagctatatatctatatatttctctcaccctattttgagacagaggcacaaagactcagcaatttttttccctcctcctcaccttccccccagtctaggtggttttgacaaagaccaaaatcccaactcagagacactgcatgcgattttactgttccaagaaaaccaggagttgcttcaatttgcagatgcttatgtgttaatacctttttctatgaaaaaagacccagcgccgtgtgcaataaaggttatgtttcta

SEQ ID NO: 466 - DNA, 6855 bp, Homo sapiens
agccgagcgctgggctgaggagcagagagagcggggcgccgagtgcgggcggctgggagcgcgctgagcgggggagaggcgctgccgcacggccggccacaggaccacctccccggagaatagggcctctttatggcatgtggctggtaactttcctcctgctcctggactctttacacaaagcccgccctgaagatgttggcaccagcctctactttgtaaatgactccttgcagcaggtgaccttttccagctccgtgggggtggtggtgccctgcccggccgcgggctcccccagcgcggcccttcgatggtacctggccacaggggacgacatctacgacgtgccgcacatccggcacgtccacgccaacgggacgctgcagctctaccccttctccccctccgccttcaatagctttatccacgacaatgactacttctgcaccgcggagaacgctgccggcaagatccggagccccaacatccgcgtcaaagcagttttcagggaaccctacaccgtccgggtggaggatcaaaggtcaatgcgtggcaacgtggccgtcttcaagtgcctcatcccctcttcagtgcaggaatatgttagcgttgtatcttgggagaaagacacagtctccatcatcccagaacacaggttttttattacctaccacggcgggctgtacatctctgacgtacagaaggaggacgccctctccacctatcgctgcatcaccaagcacaagtatagcggggagacccggcagagcaatggggcacgcctctctgtgacagaccctgctgagtcgatccccaccatcctggatggcttccactcccaggaagtgtgggccggccacaccgtggagctgccctgcaccgcctcgggctaccctatccccgccatccgctggctcaaggatggccggcccctcccggctgacagccgctggaccaagcgcatcacagggctgaccatcagcgacttgcggaccgaggacagcggcacctacatttgtgaggtcaccaacaccttcggttcggcagaggccacaggcatcctcatggtcattgatccccttcatgtgaccctgacaccaaagaagctgaagaccggcattggcagcacggtcatcctctcctgtgccctgacgggctccccagagttcaccatccgctggtatcgcaacacggagctggtgctgcctgacgaggccatctccatccgcgggctcagcaacgagacgctgctcatcacctcggcccagaagagccattccggggcctaccagtgcttcgctacccgcaaggcccagaccgcccaggactttgccatcattgcacttgaggatggcacgccccgcatcgtctcgtccttcagcgagaaggtggtcaaccccggggagcagttctcactgatgtgtgcggccaagggcgccccgccccccacggtcacctgggccctcgacgatgagcccatcgtgcgggatggcagccaccgcaccaaccagtacaccatgtcggacggcaccaccatcagccacatgaacgtcacaggcccccagatccgcgacgggggcgtgtaccggtgcacagcgcggaacttggtgggcagtgctgaatatcaggcgcgaataaacgtaagaggcccacccagcatccgggctatgcggaacatcacagcagtcgccgggcgggacacccttatcaactgcagggtcatcggctatccctactactccatcaagtggtacaaggatgccctgctgctgccagacaaccaccgccaggtggtgtttgagaatgggaccctcaagctgactgacgtgcagaagggcatggatgagggggagtacctgtgcagtgtcctcatccagccccagctctccatcagccagagcgttcacgtagccgtcaaagtgccccctctgatccagcccttcgaattcccacccgcctccatcggccagctgctctacattccctgtgtggtgtcctcgggggacatgcccatccgtatcacctggaggaaggacggacaggtgatcatctcaggctcgggcgtgaccatcgagagcaaggaattcatgagctccctgcagatctctagcgtctccctcaagcacaacggcaactatacatgcatcgccagcaacgcagccgccaccgtgagccgggagcgccagctcatcgtgcgtgtgccccctcgatttgtggtgcaacccaacaaccaggatggcatctacggcaaagctggtgtgctcaactgctcggtggacggctaccccccacccaaggtcatgtggaagcatgccaaggggagcgggaacccccagcagtaccaccctgtgcccctcactggccgcatccagatcctgcccaacagctcgctgctgatccgccacgtcctagaagaggacatcggctactacctctgccaggccagcaacggcgtaggcaccgacatcagcaagtccatgttcctcacagtcaagatcccggccatgatcacttcccaccccaacaccaccatcgccatcaagggccatgcgaaggagctaaactgcacggcacggggtgagcggcccatcatcatccgctgggagaagggggacacagtcatcgaccctgaccgcgtcatgcggtatgccatcgccaccaaggacaacggcgacgaggtcgtctccacactgaagctcaagcccgctgaccgtggggactctgtgttcttcagctgccatgccatcaactcgtatggggaggaccggggcttgatccaactcactgtgcaagagccccccgaccccccagagctggagatccgggaggtgaaggcccggagcatgaacctgcgctggacccagcgattcgacgggaacagcatcatcacgggcttcgacattgaatacaagaacaaatcagattcctgggacttcaagcagtccacacgcaacatctcccccaccatcaaccaggccaacattgtggacttgcacccggcatctgtgtacagcatccgcatgtactctttcaacaagattggccgcagtgaaccaagcaaggagctcaccatcagcactgaggaggccgctcccgatgggccccccatggatgttaccttgcagccagtgacctcacagagcatccaggtgacctggaaggcacccaagaaggagctgcagaacggtgtcatccggggctaccagattggctacagagagaacagccccggcagcaacgggcagtacagcatcgtggagatgaaggccacgggggacagcgaggtctacaccctggacaacctcaagaagttcgcccagtatggggtggtggtccaagccttcaatcgggctggcacggggccctcttccagcgagatcaatgccaccactctggaggatgtgcccagccagccccctgagaacgtccgggccctgtccatcacttctgacgtggccgtcatctcctggtcagagcccccgcgcagcaccctcaatggcgtcctcaaaggctatcgggtcatcttctggtccctctatgttgatggggagtggggcgagatgcagaacatcaccaccacgcgggagcgggtggagctgcggggcatggagaagttcaccaactacagcgtccaggtgctggcctacacccaggctggggacggcgtacgcagcagtgtgctctacatccagaccaaggaggacgttccaggtccccctgctggcatcaaagctgtcccttcatcagctagcagtgtggttgtgtcttggctcccccctaccaagcccaacggggtgatccgcaagtacaccatcttctgttccagccccgggtctggccagccggctcccagcgagtacgagacgagtccagagcagctcttctaccggatcgcccacctaaaccgcggtcagcagtatctgctgtgggtggccgccgtcacctctgccggccggggcaacagcagcgagaaggtgaccatcgagcctgctggcaaggccccagcaaagatcatctcctttgggggcaccgtgacaacaccttggatgaaagatgttcggctgccttgcaattcagtgggagatccagcccctgctgtgaagtggaccaaggacagtgaagactcggccattccagtgtccatggatgggcaccggctcatccacaccaatggcacactgctgctgcgtgcagtgaaggctgaggactctggctactacacgtgcacggccaccaacactggtggctttgacaccatcatcgtcaaccttctggtgcaagttcccccggaccagccccgcctcactgtctccaaaacctcagcttcgtccatcaccctgacctggattccaggtgacaatgggggcagctccatccgaggcttcgtgctacagtactcggtggacaacagcgaggagtggaaggatgtgttcatcagctccagcgagcgctccttcaagctggacagcctcaagtgtggcacgtggtacaaggtgaagctggcagccaagaacagcgtgggctctgggcgcatcagcgagatcatcgaggccaagacccacgggcgggagccctccttcagcaaagaccaacacctcttcacccacatcaactccacgcatgctcggcttaacctgcagggctggaacaatgggggctgccctatcacagccatcgttctggagtaccggcccaaggggacctgggcctggcagggcctccgggccaacagctccggggaggtgtttctgacggaactgcgagaggccacgtggtacgagctgcgcatgagggcttgcaacagtgcgggctgcggcaatgaaacagcccagttcgccaccctggactacgatggcagcaccattccacccatcaagtctgctcaaggtgaaggggatgatgtgaagaagctgttcaccatcggctgccctgtcatcctggccacactgggggtggcactgctcttcatcgtacgcaagaagaggaaggagaaacggctgaagcgactccgagatgcaaagagtttggcagaaatgttgataagcaagaacaatagaagctttgacacccctgtgaaagggccaccccagggcccacggctacacattgacatccccagggtccagctgctcatcgaggacaaagaaggcatcaagcaactgggagatgacaaggccaccatccctgtgacagatgctgagttcagccaagctgtcaacccacagagcttctgtactggcgtctccttgcaccacccaaccctcatccagagcacaggacccctcatcgacatgtctgacatccggccaggaaccaatccagtgtccaggaagaatgtgaagtcagcccacagcacccggaaccggtactcaagccagtggaccctgaccaagtgccaggcctccacacctgcccgcaccctcacctccgactggcgcaccgtgggctcccagcatggtgtcacggtcactgagagtgacagctacagtgccagcctgtcccaggacacagacaaaggaaggaacagcatggtgtccactgagagtgcctcttccacctacgaggagctggcccgggcctatgagcatgccaagctggaggagcagctgcagcacgccaagtttgagatcaccgagtgcttcatctctgacagttcctctgaccagatgaccacaggcaccaacgagaacgccgacagcatgacatccatgagcacaccctcagagcctggcatctgccgctttaccgcctcaccacccaagccccaggatgcggaccggggcaaaaacgtggctgtgcccatccctcaccgggccaacaagagtgactactgcaacctgcccctgtatgccaagtcagaggccttctttcgaaaggcagatggacgtgagccctgccccgtggtcccaccccgtgaggcctccatccggaacctggctcgaacctaccacacccaggctcgccacctgaccctggaccctgccagcaagtccttgggccttccccacccaggggcccccgctgccgcctccacagccaccttacctcagaggactctggccatgccagcccccccagccggcacagcccccccagcccccggccccacccctgctgagccacccaccgcccccagcgctgcccctccggcccccagcaccgagcctccacgagccgggggcccacacaccaaaatggggggctccagggactcgcttctcgagatgagcacatcgggggtagggaggtctcagaagcagggggccggggcctactccaaatcctacaccctggtgtagggcccgcaggaagagcagccacgcctggaccgcgccgcgccgcagccccacacgccagctcggctgtttttctgcattatttatattcaactgacagacaaaaaccaaccaacgacaaaacaaaaacccccaatcatgaacgcctgtacatagaactcttttgtacaaatgaaactattttcttcttctccatgaagccagggcacaaagaatttgacagtacaagtcaaatcccccaccccacaaaatatgtgtggagatatatatacatatatagacagacaggaacgcgtccacgagctatatatctatatatttctctcaccctattttgagacagaggcacaaagactcagcaatttttttccctcctcctcaccttccccccagtctaggtggttttgacaaagaccaaaatcccaactcagagacactgcatgcgattttactgttccaagaaaaccaggagttgcttcaatttgcagatgcttatgtgttaatacctttttctatgaaaaaagacccagcgccgtgtgcaataaaggttatgtttcta

SEQ ID NO: 467 - DNA, 648 bp, Homo sapiens
agccgagcgctgggctgaggagcagagagagcggggcgccgagtgcgggcggctgggagcgcgctgagcgggggagaggcgctgccgcacggccggccacaggaccacctccccggagaatagggcctctttatggcatgtggctggtaactttcctcctgctcctggactctttacacaaagcccgccctgaagatgttggcaccagcctctactttgtaaatgactccttgcagcaggtgaccttttccagctccgtgggggtggtggtgccctgcccggccgcgggctcccccagcgcggcccttcgatggtacctggccacaggggacgacatctacgacgtgccgcacatccggcacgtccacgccaacgggacgctgcagctctaccccttctccccctccgccttcaatagctttatccacgacaatgactacttctgcaccgcggagaacgctgccggcaagatccggagccccaacatccgcgtcaaagcagttttcagggaaccctacaccgtccgggtggaggatcaaaggtcaatgcgtggcaacgtggccgtcttcaagtgcctcatcccctcttcagtgcaggaatatgttagcgttgtatcttgggagaaagacacagtctccatcatcccag

SEQ ID NO: 468 - DNA, 150 bp, Homo sapiens
atgtgtccaacaaggtgtcaatgtccagcactgtgcagggcagcaacatctttgagagaacggaggtcctggcagccttgcctccccgattgaaagagatgaaaagccaggaatcggctgcaggttccaaactagtccttcggtgtgaaa

SEQ ID NO: 469 - DNA, 150 bp, Homo sapiens
tttctactgtacaccccatcccagacgaagacagtccctggatcaccgacagcacagacagaatccctgctaccactacatctacatccaccactgggacaagccatcttgtaaaatgtgcggagaaggagaaaactttctgtgtgaatg

SEQ ID NO: 470 - DNA, 485 bp, Homo sapiens
actcgccgcagcctgcgcgccttctccagtccgcggtgccatggcccccgcccgtctgttcgcgctgctgctgttcttcgtaggcggagtcgccgagtcgatccgagagactgaggtcatcgacccccaggacctcctagaaggccgatacttctccggagccctaccagacgatgaggatgtagtggggcccgggcaggaatctgatgactttgagctgtctggctctggagatctggatgacttggaagactccatgatcggccctgaagttgtccatcccttggtgcctctagataaccatatccctgagagggcagggtctgggagccaagtccccaccgaacccaagaaactagaggagaatgaggttatccccaagagaatctcacccgttgaagagagtgaggatgtgtccaacaaggtgtcaatgtccagcactgtgcagggcagcaacatctttgagagaacggaggtcctggcag

SEQ ID NO: 471 - DNA, 617 bp, Homo sapiens
agagcctgcctggagcgcgtactcagcggctctcgggtcccagcgtcccagccgcggcccgcgctcctccgccccgctcctcctcctcctcttcctcctcctcctcctctctaggcacccccgtcccctccttccagcggctgcagcccccagccccaactctccgcgcttactcctgggacgcgcgtcctcgccccatcctttgcttccttccttccttccttcttccttcctcccctggctcccgccctccctctccaggtcgccctcccggggcccgattgtctcggtgccccgctcccggcccgcgccctgccccgtctctcccttgcacttcctgagtcgcccgccgccgccgtcgcagactcgccgcgggagccccagcccaacccgagcccgacagccactgccccggctccagctccagccccacagcccgcggcgcccgcccgagggagccccggcgcccggggaaggctccagtgggctagcgcgccctcgcccagccccgcgccccagccctgcccggcccggcgaggaaggaccgggaagatgaacaacggcggcaaagccgagaaggagaacaccccgagcgaggccaaccttcaggaggagga

SEQ ID NO: 472 - DNA, 79 bp, Homo sapiens
ggtccggaccctatttgtcagtggccttcctctggatatcaaacctcgggagctctatctgcttttcagaccatttaag

SEQ ID NO: 473 - DNA, 39 bp, Homo sapiens
ggctatgagggttctcttataaagctcacatctaaacag

SEQ ID NO: 474 - DNA, 63 bp, Homo sapiens
cctgtaggttttgtcagttttgacagtcgctcagaagcagaggctgcaaagaatgctttgaat

SEQ ID NO: 475 - DNA, 151 bp, Homo sapiens
ggcatccgcttcgatcctgaaattccgcaaacactacgactagagtttgctaaggcaaacacgaagatggccaagaacaaactcgtagggactccaaaccccagtactcctctgcccaacactgtacctcagttcattgccagagagccat

SEQ ID NO: 476 - DNA, 131 bp, Homo sapiens
atgagctcacagtgcctgcactttaccccagtagccctgaagtgtgggccccgtaccctctgtacccagcggagttagcgcctgctctacctcctcctgctttcacctatcccgcttcactgcatgcccag

SEQ ID NO: 477 - DNA, 70 bp, Homo sapiens
atgcgctggctccctccctccgaggctacttctcagggctggaagtcccgtcagttctgctgaatactat

SEQ ID NO: 478 - DNA, 206 bp, Homo sapiens
acccttcagcaatggctactagaaggacgaacaattgccctcctttggaagtacggctaatagaagccctagatccgaataagatccgaataagaatatgtaatggaccaggcgcagtgcctcacgcctgtcatcccagcactttgggaggctgaggcaggcggatcacttgatgccagaagtttgagaccagcccagccaacatg

SEQ ID NO: 479 - DNA, 104 bp, Homo sapiens
gtcccaggtgtgtgatggcggctgcaatctgtcttgtgggtattaatgcaatcttcagtggtggctactgttctctagctgttctacaaaactggagcatgctg

SEQ ID NO: 480 - DNA, 1620 bp, Homo sapiens
gcttgaaaaacccttgcccagttttgatcccttcaagactttgtcacagcctctatcacacatctgtttttctcgaagaaaaaaatataattaataaaaatgttttactcttttacactgtataattgtaagaaatagcgtattatttgtgaatgcatggtctgaaatttctgtacagtttggagatattttcaaacgaaacgtaagagaagtcaacaataaaaccaagaaaagtgagtatttttataccaaacattttaagtatgctgggatggacgatcttacactggtttggatcacagtttttgttcttgacttttgaatgcttgtaattaaaaatatctatttttttcctctgaagtaagttgcatgtttgaggcatgtttccaaatattatcaaaatatcctgaattgtattgtgaatatatagaaatctgtgcctggccggagtccagggtaaattagtagcatggtgttagatgttagaaacagaactgttatttgcagtgttaggtctaggatcccagttctagtaggacagccctgcaagacaatcaaccagaagcctccaggagcttctacctatggcttattcacaactgggcaagaaaacatcattggtaagaactgctgagtgtgcccttagaaagccctagtagctccagctgtgactatatcaactgtgtgccaagtgtgactttgtacagttttatgtttccactctcctgtatgtgtagccactcgatgcctaacctaccttccacaagccagccccgcatccctgctcccgcagtgtaagtgcagagcctgcctcactggtaagggaaaaccttggcttgggaggccagccctggcccttgaaggggttggctgtgcccagcccacctggctgcagtgggcagctcatgtctgtatctccaaagtgatgtttgtttgcaaaacaccggctgaactgagctggtgttgccaactcttggcagcactgggccaaaccgaccacataccatgagctcccaaatggcgtgtgctcactgtgagacgtcctgccacaccccacaggagacggaggcagtgggcatttggaaccaattctattcagacttcgtcaaagccaaagtcagtctggtgttgtcagttgacacatctccagagttcatgacagctcaacctctccccttgtacagaagccatttttgtaaaaccacactgacctaaattcagctgccaaacacagtctttcctattgatccgctcggcttatgttgaaaatttcaatgtcatgattacctggttggtttgggtttttgttttgttattttccattagaaatataaagatgtcaagaagcttttaaaggtcaacacaaaaaaccaaggccaggagtgaggggctctttcttaccgtaaataaggggaaaaggcagttagctcaaggacttgtgacggatccactttggtgttcaaggacctgcttatgccctcagtgccaatcggctcttggtgagatgactgtactcctaaggaaaatagccacttctgcagtctattatgcttttataactgtttaaaggtacttttctattgtcatttttaaaaaataaagtgcttattccagctgtca

SEQ ID NO: 481 - DNA, 3080 bp, Homo sapiens
agagcctgcctggagcgcgtactcagcggctctcgggtcccagcgtcccagccgcggcccgcgctcctccgccccgctcctcctcctcctcttcctcctcctcctcctctctaggcacccccgtcccctccttccagcggctgcagcccccagccccaactctccgcgcttactcctgggacgcgcgtcctcgccccatcctttgcttccttccttccttccttcttccttcctcccctggctcccgccctccctctccaggtcgccctcccggggcccgattgtctcggtgccccgctcccggcccgcgccctgccccgtctctcccttgcacttcctgagtcgcccgccgccgccgtcgcagactcgccgcgggagccccagcccaacccgagcccgacagccactgccccggctccagctccagccccacagcccgcggcgcccgcccgagggagccccggcgcccggggaaggctccagtgggctagcgcgccctcgcccagccccgcgccccagccctgcccggcccggcgaggaaggaccgggaagatgaacaacggcggcaaagccgagaaggagaacaccccgagcgaggccaaccttcaggaggaggaggtccggaccctatttgtcagtggccttcctctggatatcaaacctcgggagctctatctgcttttcagaccatttaagggctatgagggttctcttataaagctcacatctaaacagcctgtaggttttgtcagttttgacagtcgctcagaagcagaggctgcaaagaatgctttgaatggcatccgcttcgatcctgaaattccgcaaacactacgactagagtttgctaaggcaaacacgaagatggccaagaacaaactcgtagggactccaaaccccagtactcctctgcccaacactgtacctcagttcattgccagagagccatatgagctcacagtgcctgcactttaccccagtagccctgaagtgtgggccccgtaccctctgtacccagcggagttagcgcctgctctacctcctcctgctttcacctatcccgcttcactgcatgcccagatgcgctggctccctccctccgaggctacttctcagggctggaagtcccgtcagttctgctgaatactatacccttcagcaatggctactagaaggacgaacaattgccctcctttggaagtacggctaatagaagccctagatccgaataagatccgaataagaatatgtaatggaccaggcgcagtgcctcacgcctgtcatcccagcactttgggaggctgaggcaggcggatcacttgatgccagaagtttgagaccagcccagccaacatggtcccaggtgtgtgatggcggctgcaatctgtcttgtgggtattaatgcaatcttcagtggtggctactgttctctagctgttctacaaaactggagcatgctggcttgaaaaacccttgcccagttttgatcccttcaagactttgtcacagcctctatcacacatctgtttttctcgaagaaaaaaatataattaataaaaatgttttactcttttacactgtataattgtaagaaatagcgtattatttgtgaatgcatggtctgaaatttctgtacagtttggagatattttcaaacgaaacgtaagagaagtcaacaataaaaccaagaaaagtgagtatttttataccaaacattttaagtatgctgggatggacgatcttacactggtttggatcacagtttttgttcttgacttttgaatgcttgtaattaaaaatatctatttttttcctctgaagtaagttgcatgtttgaggcatgtttccaaatattatcaaaatatcctgaattgtattgtgaatatatagaaatctgtgcctggccggagtccagggtaaattagtagcatggtgttagatgttagaaacagaactgttatttgcagtgttaggtctaggatcccagttctagtaggacagccctgcaagacaatcaaccagaagcctccaggagcttctacctatggcttattcacaactgggcaagaaaacatcattggtaagaactgctgagtgtgcccttagaaagccctagtagctccagctgtgactatatcaactgtgtgccaagtgtgactttgtacagttttatgtttccactctcctgtatgtgtagccactcgatgcctaacctaccttccacaagccagccccgcatccctgctcccgcagtgtaagtgcagagcctgcctcactggtaagggaaaaccttggcttgggaggccagccctggcccttgaaggggttggctgtgcccagcccacctggctgcagtgggcagctcatgtctgtatctccaaagtgatgtttgtttgcaaaacaccggctgaactgagctggtgttgccaactcttggcagcactgggccaaaccgaccacataccatgagctcccaaatggcgtgtgctcactgtgagacgtcctgccacaccccacaggagacggaggcagtgggcatttggaaccaattctattcagacttcgtcaaagccaaagtcagtctggtgttgtcagttgacacatctccagagttcatgacagctcaacctctccccttgtacagaagccatttttgtaaaaccacactgacctaaattcagctgccaaacacagtctttcctattgatccgctcggcttatgttgaaaatttcaatgtcatgattacctggttggtttgggtttttgttttgttattttccattagaaatataaagatgtcaagaagcttttaaaggtcaacacaaaaaaccaaggccaggagtgaggggctctttcttaccgtaaataaggggaaaaggcagttagctcaaggacttgtgacggatccactttggtgttcaaggacctgcttatgccctcagtgccaatcggctcttggtgagatgactgtactcctaaggaaaatagccacttctgcagtctattatgcttttataactgtttaaaggtacttttctattgtcatttttaaaaaataaagtgcttattccagctgtca

SEQ ID NO: 482 - DNA, 949 bp, Homo sapiens
agagcctgcctggagcgcgtactcagcggctctcgggtcccagcgtcccagccgcggcccgcgctcctccgccccgctcctcctcctcctcttcctcctcctcctcctctctaggcacccccgtcccctccttccagcggctgcagcccccagccccaactctccgcgcttactcctgggacgcgcgtcctcgccccatcctttgcttccttccttccttccttcttccttcctcccctggctcccgccctccctctccaggtcgccctcccggggcccgattgtctcggtgccccgctcccggcccgcgccctgccccgtctctcccttgcacttcctgagtcgcccgccgccgccgtcgcagactcgccgcgggagccccagcccaacccgagcccgacagccactgccccggctccagctccagccccacagcccgcggcgcccgcccgagggagccccggcgcccggggaaggctccagtgggctagcgcgccctcgcccagccccgcgccccagccctgcccggcccggcgaggaaggaccgggaagatgaacaacggcggcaaagccgagaaggagaacaccccgagcgaggccaaccttcaggaggaggaggtccggaccctatttgtcagtggccttcctctggatatcaaacctcgggagctctatctgcttttcagaccatttaagggctatgagggttctcttataaagctcacatctaaacagcctgtaggttttgtcagttttgacagtcgctcagaagcagaggctgcaaagaatgctttgaatggcatccgcttcgatcctgaaattccgcaaacactacgactagagtttgctaaggcaaacacgaagatggccaagaacaaactcgtagggactccaaaccccagtactcctctgcccaacactgtacctcagttcattgccagagagccat

SEQ ID NO: 483 - DNA, 237 bp, Homo sapiens
actccgagagccgcagcggcagcggcgcgtcctgcctgcagagagccaggccggagaagccgagcggcgcagaggacgccagggcgcgcgccgcagccacccaccctccggaccgcggcagctgctgacccgccatcgccatggcccgcgggaaagccaaggaggagggcagctggaagaaattcatctggaactcagagaagaaggagtttctgggcaggaccggtggcagttggt

SEQ ID NO: 484 - DNA, 129 bp, Homo sapiens
ttaagatccttctattctacgtaatattttatggctgcctggctggcatcttcatcggaaccatccaagtgatgctgctcaccatcagtgaatttaagcccacatatcaggaccgagtggccccgccag

SEQ ID NO: 485 - DNA, 156 bp, Homo sapiens
gattaacacagattcctcagatccagaagactgaaatttcctttcgtcctaatgatcccaagagctatgaggcatatgtactgaacatagttaggttcctggaaaagtacaaagattcagcccagagggatgacatgatttttgaagattgtggcg

SEQ ID NO: 486 - DNA, 185 bp, Homo sapiens
atgtgcccagtgaaccgaaagaacgaggagactttaatcatgaacgaggagagcgaaaggtctgcagattcaagcttgaatggctgggaaattgctctggattaaatgatgaaacttatggctacaaagagggcaaaccgtgcattattataaagctcaaccgagttctaggcttcaaacctaag

SEQ ID NO: 487 - DNA, 81 bp, Homo sapiens
cctcccaagaatgagtccttggagacttacccagtgatgaagtataacccaaatgtccttcccgttcagtgcactggcaag

SEQ ID NO: 488 - DNA, 1428 bp, Homo sapiens
cgagatgaagataaggataaagttggaaatgtggagtattttggactgggcaactcccctggttttcctctgcagtattatccgtactatggcaaactcctgcagcccaaatacctgcagcccctgctggccgtacagttcaccaatcttaccatggacactgaaattcgcatagagtgtaaggcgtacggtgagaacattgggtacagtgagaaagaccgttttcagggacgttttgatgtaaaaattgaagttaagagctgatcacaagcacaaatctttcccactagccatttaataagttaaaaaaagatacaaaaacaaaaacctactagtcttgaacaaactgtcatacgtatgggacctacacttaatctatatgctttacactagctttctgcatttaataggttagaatgtaaattaaagtgtagcaatagcaacaaaatatttattctactgtaaatgacaaaagaaaaagaaaaattgagccttgggacgtgcccatttttactgtaaattatgattccgtaactgacttgtagtaagcagtgtttctggcccctaagtattgctgccttgtgtattttatttagtgtacagtactacaggtgcatactctggtcatttttcaagccatgttttattgtatctgttttctactttatgtgagcaaggtttgctgtccaaggtgtaaatattcaacgggaataaaactggcatggtaattttttttttttttttttttttgttttttggctctttcaaaggtaatggcccatcgatgagcatttttaacatactccatagtcttttcctgtggtgttaggtctttatttttatttttttcctgggggctggggtgggggtttgtcatgggggaactgccctttaaattttaagtgacactacagaaaaacacaaaaaggtgatgggttgtgttatgcttgtattgaatgctgtcttgacatctcttgccttgtcctccggtatgttctaaagctgtgtctgagatctggatctgcccatcactttggctagtgacagggctaattaatttgctttatacattttcttttactttccttttttcctttctggaggcatcacatgctggtgctgtgtctttatgaatgttttaaccattttcatggtggaagaattttatatttatgcagttgtacaattttatttttttctgcaagaaaaagtgtaatgtatgaaataaaccaaagtcacttgtttgaaaataaatctttattttgaactttataaaaagcaatgcagtaccccatagactggtgttaaatgttgtctacagtgcaaaatccatgttctaacatatgtaataattgccaggagtacagtgctcttgttgatcttgtattcagtcaggttaaaacaacggacaataaaagaatgaacaca

SEQ ID NO: 489 - DNA, 2216 bp, Homo sapiens
actccgagagccgcagcggcagcggcgcgtcctgcctgcagagagccaggccggagaagccgagcggcgcagaggacgccagggcgcgcgccgcagccacccaccctccggaccgcggcagctgctgacccgccatcgccatggcccgcgggaaagccaaggaggagggcagctggaagaaattcatctggaactcagagaagaaggagtttctgggcaggaccggtggcagttggtttaagatccttctattctacgtaatattttatggctgcctggctggcatcttcatcggaaccatccaagtgatgctgctcaccatcagtgaatttaagcccacatatcaggaccgagtggccccgccaggattaacacagattcctcagatccagaagactgaaatttcctttcgtcctaatgatcccaagagctatgaggcatatgtactgaacatagttaggttcctggaaaagtacaaagattcagcccagagggatgacatgatttttgaagattgtggcgatgtgcccagtgaaccgaaagaacgaggagactttaatcatgaacgaggagagcgaaaggtctgcagattcaagcttgaatggctgggaaattgctctggattaaatgatgaaacttatggctacaaagagggcaaaccgtgcattattataaagctcaaccgagttctaggcttcaaacctaagcctcccaagaatgagtccttggagacttacccagtgatgaagtataacccaaatgtccttcccgttcagtgcactggcaagcgagatgaagataaggataaagttggaaatgtggagtattttggactgggcaactcccctggttttcctctgcagtattatccgtactatggcaaactcctgcagcccaaatacctgcagcccctgctggccgtacagttcaccaatcttaccatggacactgaaattcgcatagagtgtaaggcgtacggtgagaacattgggtacagtgagaaagaccgttttcagggacgttttgatgtaaaaattgaagttaagagctgatcacaagcacaaatctttcccactagccatttaataagttaaaaaaagatacaaaaacaaaaacctactagtcttgaacaaactgtcatacgtatgggacctacacttaatctatatgctttacactagctttctgcatttaataggttagaatgtaaattaaagtgtagcaatagcaacaaaatatttattctactgtaaatgacaaaagaaaaagaaaaattgagccttgggacgtgcccatttttactgtaaattatgattccgtaactgacttgtagtaagcagtgtttctggcccctaagtattgctgccttgtgtattttatttagtgtacagtactacaggtgcatactctggtcatttttcaagccatgttttattgtatctgttttctactttatgtgagcaaggtttgctgtccaaggtgtaaatattcaacgggaataaaactggcatggtaattttttttttttttttttttttgttttttggctctttcaaaggtaatggcccatcgatgagcatttttaacatactccatagtcttttcctgtggtgttaggtctttatttttatttttttcctgggggctggggtgggggtttgtcatgggggaactgccctttaaattttaagtgacactacagaaaaacacaaaaaggtgatgggttgtgttatgcttgtattgaatgctgtcttgacatctcttgccttgtcctccggtatgttctaaagctgtgtctgagatctggatctgcccatcactttggctagtgacagggctaattaatttgctttatacattttcttttactttccttttttcctttctggaggcatcacatgctggtgctgtgtctttatgaatgttttaaccattttcatggtggaagaattttatatttatgcagttgtacaattttatttttttctgcaagaaaaagtgtaatgtatgaaataaaccaaagtcacttgtttgaaaataaatctttattttgaactttataaaaagcaatgcagtaccccatagactggtgttaaatgttgtctacagtgcaaaatccatgttctaacatatgtaataattgccaggagtacagtgctcttgttgatcttgtattcagtcaggttaaaacaacggacaataaaagaatgaacaca

SEQ ID NO: 490 - DNA, 366 bp, Homo sapiens
actccgagagccgcagcggcagcggcgcgtcctgcctgcagagagccaggccggagaagccgagcggcgcagaggacgccagggcgcgcgccgcagccacccaccctccggaccgcggcagctgctgacccgccatcgccatggcccgcgggaaagccaaggaggagggcagctggaagaaattcatctggaactcagagaagaaggagtttctgggcaggaccggtggcagttggtttaagatccttctattctacgtaatattttatggctgcctggctggcatcttcatcggaaccatccaagtgatgctgctcaccatcagtgaatttaagcccacatatcaggaccgagtggccccgccag

SEQ ID NO: 491 - DNA, 33 bp, Artificial Sequence
  T7 promotor
cagagatgcataatacgactcactatagggaga